(19) United States
(12) Patent Application Publication
(10) Pub. No.: US 2024/0034736 A1
(43) Pub. Date: Feb. 1, 2024

(54) HSD17B13 INHIBITORS AND USES THEREOF

(71) Applicant: FL2022-001, Inc., San Francisco, CA (US)

(72) Inventors: Andrew R. Hudson, San Diego, CA (US); Steven P. Govek, San Diego, CA (US); Johnny Y. Nagasawa, San Diego, CA (US); Iriny Botrous, San Diego, CA (US); Nicholas D. Smith, San Diego, CA (US); Karensa L. Fasanya, San Diego, CA (US)

(21) Appl. No.: 18/247,135

(22) PCT Filed: Sep. 29, 2021
(86) PCT No.: PCT/US21/52674

§ 371 Date: Mar. 29, 2023

Related U.S. Application Data
(60) Provisional application No. 63/085,846, filed on Sep. 30, 2020.

(51) Int. Cl. C07D 487/04; C07D 231/56; C07D 401/04; C07D 471/04; C07D 487/10; A61P 1/16

(52) U.S. Cl. CPC C07D 487/04; C07D 231/56; C07D 401/04; C07D 471/04; C07D 487/10; A61P 1/16

(57) ABSTRACT

Described herein are compounds that are HSD17B13 inhibitors, methods of making such compounds, pharmaceutical compositions and medicaments comprising such compounds, and methods of using such compounds in the treatment of conditions, diseases, or disorders associated with HSD17B13 activity.

### CROSS-REFERENCE

This application claims benefit of U.S. Provisional Patent Application No. 63/085,846, filed on Sep. 30, 2020 which is incorporated herein by reference in its entirety.

### FIELD OF THE INVENTION

Described herein are compounds that are hydroxysteroid 17p-dehydrogenase 13 (HSD17B13) inhibitors, methods of making such compounds, pharmaceutical compositions and medicaments comprising such compounds, and methods of using such compounds in the treatment of conditions, diseases, or disorders associated with HSD17B13 activity.

### BACKGROUND OF THE INVENTION

Hydroxysteroid dehydrogenase 17β13 (HSD17b13) is a member of the short-chain dehydrogenase/reductase enzymes highly expressed in the liver on lipid droplets. It has been shown to oxidize retinol, steroids such as estradiol, and bio-active lipids like leukotriene B4. Loss of HSD17b13 expression and enzymatic activity is associated with decreased incidence of liver disease. Inhibition of HSD17b 13 enzymatic activity can be used for the treatment of liver diseases that result in hepatic inflammation, fibrosis, cirrhosis, and development of hepatocellular carcinoma.

### SUMMARY OF THE INVENTION

In one aspect, described herein is a compound of Formula (I′), or a pharmaceutically acceptable salt or solvate thereof:

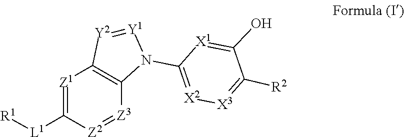

wherein:
X¹, X², and X³ are each independently CR³ or N;
Y¹ and Y² are each independently CR⁴ or N;
Z¹, Z², and Z³ are each independently CR⁵ or N;
L¹ is selected from a bond, —O—, —N(R¹⁰)—, —S(O)₂—, —C(R¹⁰)(R¹¹)N(R¹⁰)—, and —N(R¹⁰)C(R¹⁰)(R¹¹)—;
R¹ is selected from:
   a) C₃₋₈cycloalkyl and C₂₋₉heterocycloalkyl, wherein C₃₋₈cycloalkyl and C₂₋₉heterocycloalkyl are optionally substituted with one, two, or three R⁶; or
   b) C₆₋₁₀aryl and C₁₋₉heteroaryl, wherein C₆₋₁₀aryl and C₁₋₉heteroaryl are substituted with one, two, or three R⁷;
R² is selected from H, halogen, —CN, C₁₋₆alkyl, C₁₋₆haloalkyl, C₂₋₆alkenyl, C₂₋₆alkynyl, C₃₋₆ cycloalkyl, C₂₋₉heterocycloalkyl, C₆₋₁₀aryl, C₁₋₉heteroaryl, —OR¹⁰, —SR¹⁰, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)C(O)N(R¹⁰)(R¹¹), —N(R¹²)C(O)R¹³, —S(O)₂R¹³, —S(O)₂N(R¹⁰)(R¹¹)—, S(═O)(═NH)N(R¹⁰)(R¹¹), —CH₂C(O)N(R¹⁰)(R¹¹), —CH₂N(R¹²)C(O)R¹³, —CH₂S(O)₂R¹³, and —CH₂S(O)₂N(R¹⁰)(R¹¹), wherein C₁₋₆alkyl, C₂₋₆alkenyl, C₂₋₆alkynyl, C₃₋₆ cycloalkyl, C₂₋₉heterocycloalkyl, C₆₋₁₀aryl, and C₁₋₉heteroaryl are optionally substituted with one, two, or three groups selected from halogen, C₁₋₆alkyl, C₁₋₆ haloalkyl, —OR¹⁰, and —N(R¹⁰)(R¹¹);
each R³ and each R⁴ are each independently selected from H, halogen, —CN, C₁₋₆alkyl, C₁₋₆ haloalkyl, C₂₋₆alkenyl, C₂₋₆alkynyl, C₃₋₆cycloalkyl, C₂₋₉heterocycloalkyl, C₁₋₉ heteroaryl, —OR¹⁰, —SR¹⁰, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R¹³, —S(O)₂N(R¹⁰)(R¹¹)—, S(═O)(═NH)N(R¹⁰)(R¹¹), —CH₂C(O)N(R¹⁰)(R¹¹), —CH₂N(R¹²)C(O)R¹³, —CH₂S(O)₂R¹³, and —CH₂S(O)₂N(R¹⁰)(R¹¹), wherein C₁₋₆alkyl, C₂₋₆alkenyl, C₂₋₆alkynyl, C₃₋₆cycloalkyl, C₂₋₉heterocycloalkyl, and C₁₋₉heteroaryl are optionally substituted with one, two, or three groups selected from halogen, C₁₋₆alkyl, C₁₋₆haloalkyl, —OR¹⁰, and —N(R¹⁰)(R¹¹);
each R⁵ is independently selected from H, halogen, —CN, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹⁰, —SR¹⁰, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R¹³, —S(O)₂N(R¹⁰)(R¹¹)—, S(═O)(═NH)N(R¹⁰)(R¹¹), —CH₂C(O)N(R¹⁰)(R¹¹), —CH₂N(R¹²)C(O)R¹³, —CH₂S(O)₂R¹³, and —CH₂S(O)₂N(R¹⁰)(R¹¹), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three groups selected from halogen, C₁₋₆alkyl, C₁₋₆haloalkyl, —OR¹⁰, and —N(R¹⁰)(R¹¹);
each R⁶ and each R⁷ are each independently selected from halogen, oxo, —CN, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹⁰, —SR¹⁰, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R¹³, —S(O)₂N(R¹⁰)(R¹¹)—, S(═O)(═NH)N(R¹⁰)(R¹¹), —CH₂C(O)N(R¹⁰)(R¹¹), —CH₂N(R¹²)C(O)R¹³, —CH₂S(O)₂R¹³, and —CH₂S(O)₂N(R¹⁰)(R¹¹), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three groups selected from halogen, C₁₋₆alkyl, C₁₋₆haloalkyl, —OR¹⁰, —C(O)OR¹⁰, and —N(R¹⁰)(R¹¹);
each R¹⁰ is independently selected from hydrogen, C₁₋₆alkyl, C₁₋₆ haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from halogen, C₁₋₆alkyl, C₁₋₆haloalkyl, C₁₋₆alkoxy, C₃₋₆cycloalkyl, C₂₋₉heterocycloalkyl, C₆₋₁₀aryl, C₁₋₉heteroaryl, —N(R¹¹)(R¹²), and —C(O)OR¹¹;
each R¹¹ is independently selected from hydrogen, C₁₋₆alkyl, and C₁₋₆haloalkyl;
each R¹² is independently selected from hydrogen, C₁₋₆alkyl, and C₁₋₆haloalkyl; and
each R¹³ is independently selected C₁₋₆alkyl, C₁₋₆haloalkyl, C₂₋₆alkenyl, C₂₋₆alkynyl, C₃₋₆ cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three groups selected from halogen, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl.

In another aspect, described herein is a compound of Formula (I), or a pharmaceutically acceptable salt or solvate thereof:

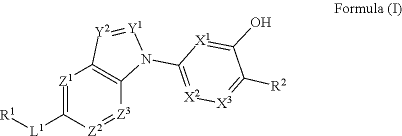

wherein:
   X¹, X², and X³ are each independently CR³ or N;
   Y¹ and Y² are each independently CR⁴ or N;
   Z¹, Z², and Z³ are each independently CR⁵ or N;
   L¹ is selected from a bond, —O—, —N(R¹⁰)—, —S(O)₂—, —C(R¹⁰)(R¹¹)N(R¹⁰)—, and —N(R¹⁰)C(R¹⁰)(R¹¹)—;
   R¹ is selected from:
      a) C₃₋₈cycloalkyl and C₂₋₉heterocycloalkyl, wherein C₃₋₈cycloalkyl and C₂₋₉heterocycloalkyl are optionally substituted with one, two, or three R⁶; or
      b) C₆₋₁₀aryl and C₁₋₉heteroaryl, wherein C₆₋₁₀aryl and C₁₋₉heteroaryl are substituted with one, two, or three R⁷;
   R² is selected from H, halogen, —CN, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹⁰, —SR¹⁰, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R¹³, —S(O)₂N(R¹⁰)(R¹¹)—, S(═O)(═NH)N(R¹⁰)(R¹¹), —CH₂C(O)N(R¹⁰)(R¹¹), —CH₂N(R¹²)C(O)R¹³, —CH₂S(O)₂R¹³, and —CH₂S(O)₂N(R¹⁰)(R¹¹), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three groups selected from halogen, C₁₋₆alkyl, C₁₋₆haloalkyl, —OR¹⁰, and —N(R¹⁰)(R¹¹);
   each R³, each R⁴, and each R⁵ are each independently selected from H, halogen, —CN, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹⁰, —SR¹⁰, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R¹³, —S(O)₂N(R¹⁰)(R¹¹)—, S(═O)(═NH)N(R¹⁰)(R¹¹), —CH₂C(O)N(R¹⁰)(R¹¹), —CH₂N(R¹²)C(O)R¹³, —CH₂S(O)₂R¹³, and —CH₂S(O)₂N(R¹⁰)(R¹¹), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from halogen, C₁₋₆ alkyl, C₁₋₆haloalkyl, —OR¹⁰, and —N(R¹⁰)(R¹¹);
   each R⁶ and each R⁷ are each independently selected from halogen, oxo, —CN, C₁₋₆ alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹⁰, —SR¹⁰, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R¹³, —S(O)₂N(R¹⁰)(R¹¹)—, S(═O)(═NH)N(R¹⁰)(R¹¹), —CH₂C(O)N(R¹⁰)(R¹¹), —CH₂N(R¹²)C(O)R¹³, —CH₂S(O)₂R¹³, and —CH₂S(O)₂N(R¹⁰)(R¹¹), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from halogen, C₁₋₆ alkyl, C₁₋₆haloalkyl, —OR¹⁰, and —N(R¹⁰)(R¹¹);
   each R¹⁰ is independently selected from hydrogen, C₁₋₆alkyl, C₁₋₆ haloalkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from halogen, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl;
   each R¹¹ is independently selected from hydrogen, C₁₋₆alkyl, and C₁₋₆haloalkyl;
   each R¹² is independently selected from hydrogen, C₁₋₆alkyl, and C₁₋₆haloalkyl; and
   each R¹³ is independently selected C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆ alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from halogen, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl.

In another aspect, described herein is a compound of Formula (II), or a pharmaceutically acceptable salt or solvate thereof:

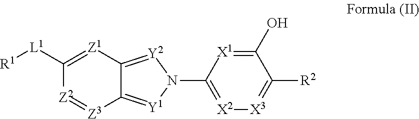

wherein:
X¹, X², and X³ are each independently CR³ or N;
Y¹ and Y² are each independently CR⁴ or N;
Z¹, Z², and Z³ are each independently CR⁵ or N;
L¹ is selected from a bond, —O—, —N(R¹⁰)—, —C(R¹⁰)(R¹¹)N(R¹⁰)—, and —N(R¹⁰)C(R¹⁰)(R¹¹)—;
R¹ is selected from:
   a) C₃₋₈cycloalkyl and C₂₋₉heterocycloalkyl, wherein C₃₋₈cycloalkyl and C₂₋₉heterocycloalkyl are optionally substituted with one, two, or three R⁶; or
   b) C₆₋₁₀aryl and C₁₋₉heteroaryl, wherein C₆₋₁₀aryl and C₁₋₉heteroaryl are substituted with one, two, or three R⁷;
R² is selected from H, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹⁰, —SR¹⁰, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R¹³, —S(O)₂N(R¹⁰)(R¹¹)—, S(═O)(═NH)N(R¹⁰)(R¹¹), —CH₂C(O)N(R¹⁰)(R¹¹), —CH₂N(R¹²)C(O)R¹³, —CH₂S(O)₂R¹³, and —CH₂S(O)₂N(R¹⁰)(R¹¹), wherein C₁₋₆alkyl, C₂₋₆alkenyl, C₂₋₆alkynyl, C₃₋₆ cycloalkyl, C₂₋₉heterocycloalkyl, C₆₋₁₀aryl, and C₁₋₉heteroaryl are optionally substituted with one, two, or three groups selected from halogen, C₁₋₆alkyl, C₁₋₆ haloalkyl, —OR¹⁰, and —N(R¹⁰)(R¹¹);
each R³ is independently selected from H, halogen, —CN, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹⁰, —SR¹⁰, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R¹³, —S(O)₂N(R¹⁰)(R¹¹)—, S(═O)(═NH)N(R¹⁰)(R¹¹), —CH₂C(O)N(R¹⁰)(R¹¹), —CH₂N(R¹²)C(O)R¹³, —CH₂S(O)₂R¹³, and —CH₂S(O)₂N(R¹⁰)(R¹¹), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three groups selected from halogen, C₁₋₆alkyl, C₁₋₆haloalkyl, —OR¹⁰, and —N(R¹⁰)(R¹¹);
each R⁴ and each R⁵ are each independently selected from H, halogen, —CN, C₁₋₆alkyl, C₁₋₆ haloalkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —SR¹⁰, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R¹³, —S(O)₂N(R¹⁰)(R¹¹)—, S(═O)(═NH)N(R¹⁰)(R¹¹), —CH₂C(O)N(R¹⁰)(R¹¹), —CH₂N(R¹²)C(O)R¹³, —CH₂S(O)₂R¹³, and —CH₂S(O)₂N(R¹⁰)(R¹¹), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three groups selected from halogen, C₁₋₆alkyl, C₁₋₆haloalkyl, —OR¹⁰, and —N(R¹⁰)(R¹¹);
each R⁶ and each R⁷ are each independently selected from halogen, oxo, —CN, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹⁰, —SR¹⁰, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R¹³, —S(O)₂N(R¹⁰)(R¹¹)—, S(═O)(═NH)N(R¹⁰)(R¹¹), —CH₂C(O)N(R¹⁰)(R¹¹), —CH₂N(R¹²)C(O)R¹³, —CH₂S(O)₂R¹³, and —CH₂S(O)₂N(R¹⁰)(R¹¹), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three groups selected from halogen, C₁₋₆alkyl, C₁₋₆haloalkyl, —OR¹⁰, and —N(R¹⁰)(R¹¹);
each R¹⁰ is independently selected from hydrogen, C₁₋₆alkyl, C₁₋₆ haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from halogen, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl;
each R¹¹ is independently selected from hydrogen, C₁₋₆alkyl, and C₁₋₆haloalkyl;
each R¹² is independently selected from hydrogen, C₁₋₆alkyl, and C₁₋₆haloalkyl; and
each R¹³ is independently selected C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from halogen, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉ heteroaryl.

In some embodiments is a compound of Formula (I′), (I), or (II), or a pharmaceutically acceptable salt or solvate thereof, wherein X¹, X², and X³ are CR³. In some embodiments is a compound of Formula (I′), (I), or (II), or a pharmaceutically acceptable salt or solvate thereof, wherein ring Y² is CR⁴.

In some embodiments is a compound of Formula (I′) or (I), or a pharmaceutically acceptable salt or solvate thereof, having the structure of Formula (Ia′):

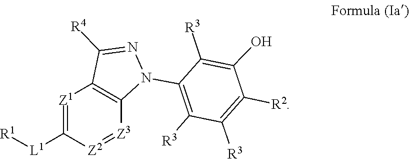

In some embodiments is a compound of Formula (I′), (I), (Ia′), or (II), or a pharmaceutically acceptable salt or solvate thereof, wherein Z¹, Z², and Z³ are CR⁵. In some embodiments is a compound of Formula (I′), (I), (Ia′), or (II), or a pharmaceutically acceptable salt or solvate thereof, wherein Z¹ is N; and Z² and Z³ are CR⁵. In some embodiments is a compound of Formula (I′), (I), (Ia′), or (II), or a pharmaceutically acceptable salt or solvate thereof, wherein Z² is N; and Z¹ and Z³ are CR⁵. In some embodiments is a compound of Formula (I′), (I), (Ia′), or (II), or a pharmaceutically acceptable salt or solvate thereof, wherein Z³ is N; and Z¹ and Z² are CR⁵. In some embodiments is a compound of Formula (I′), (I), (Ia′), or (II), or a pharmaceutically acceptable salt or solvate thereof, wherein Z¹ is CR⁵; and Z² and Z³ are N. In some embodiments is a compound of Formula (I′), (I), (Ia′), or (II), or a pharmaceutically acceptable salt or solvate thereof, wherein Z² is CR⁵; and Z¹ and Z³ are N. In some embodiments is a compound of Formula (I′), (I), (Ia′), or (II), or a pharmaceutically acceptable salt or solvate thereof, wherein Z³ is CR⁵; and Z¹ and Z² are N. In some embodiments is a compound of Formula (I′), (I), (Ia′), or (II), or a pharmaceutically acceptable salt or solvate thereof, wherein L¹ is a bond. In some embodiments is a compound of Formula (I′), (I), (Ia′), or (II), or a pharmaceutically acceptable salt or solvate thereof, wherein L¹ is —O—. In some embodiments is a compound of Formula (I′), (I), (Ia′), or (II), or a pharmaceutically acceptable salt or solvate thereof, wherein L¹ is —N(R¹⁰)—. In some embodiments is a compound of Formula (I′), (I), (Ia′), or (II), or a pharmaceutically acceptable salt or solvate thereof, wherein L¹ is —N(H)—. In some embodiments is a compound of Formula (I′), (I), (Ia′), or (II), or a pharmaceutically acceptable salt or solvate thereof, wherein L¹ is —N(CH₃)—. In some embodiments is a compound of Formula (I′), (I), (Ia′), or (II), or a pharmaceutically acceptable salt or solvate thereof, wherein R¹ is selected from C₃₋₈cycloalkyl and C₂₋₉heterocycloalkyl, wherein C₃₋₈cycloalkyl and C₂₋₉heterocycloalkyl are optionally substituted with one, two, or three R⁶. In some embodiments is a compound of Formula (I′), (I), (Ia′), or (II), or a pharmaceutically acceptable salt or solvate thereof, wherein R¹ is C₂₋₉heterocycloalkyl optionally substituted with one, two, or three R⁶. In some embodiments is a compound of Formula (I′), (I), (Ia′), or (II), or a pharmaceutically acceptable salt or solvate thereof, wherein R¹ is C₂₋₉heterocycloalkyl selected from piperidinyl, piperazinyl, morpholinyl, tetrahydropyranyl, tetrahydrofuranyl, pyrrolidinyl, oxetanyl, azetidinyl, aziridinyl, azepanyl, diazepanyl, 6-azaspiro[2.5]octanyl, 4,7-diazaspiro[2.5]octanyl, 7-oxa-4-azaspiro[2.5]octanyl, 5,8-diazaspiro[3.5]nonanyl, 8-oxa-5-azaspiro[3.5]nonanyl, or 2,6-diazaspiro[3.3]heptanyl, wherein piperidinyl, piperazinyl, morpholinyl, tetrahydropyranyl, tetrahydrofuranyl, pyrrolidinyl, oxetanyl, azetidinyl, aziridinyl, azepanyl, diazepanyl, 6-azaspiro[2.5]octanyl, 4,7-diazaspiro[2.5]octanyl, 7-oxa-4-azaspiro[2.5]octanyl, 5,8-diazaspiro[3.5]nonanyl, 8-oxa-5-azaspiro[3.5]nonanyl, or 2,6-diazaspiro[3.3]heptanyl are optionally substituted with one, two, or three R⁶. In some embodiments is a compound of Formula (I′), (I), (Ia′), or (II), or a pharmaceutically acceptable salt or solvate thereof, wherein R¹ is

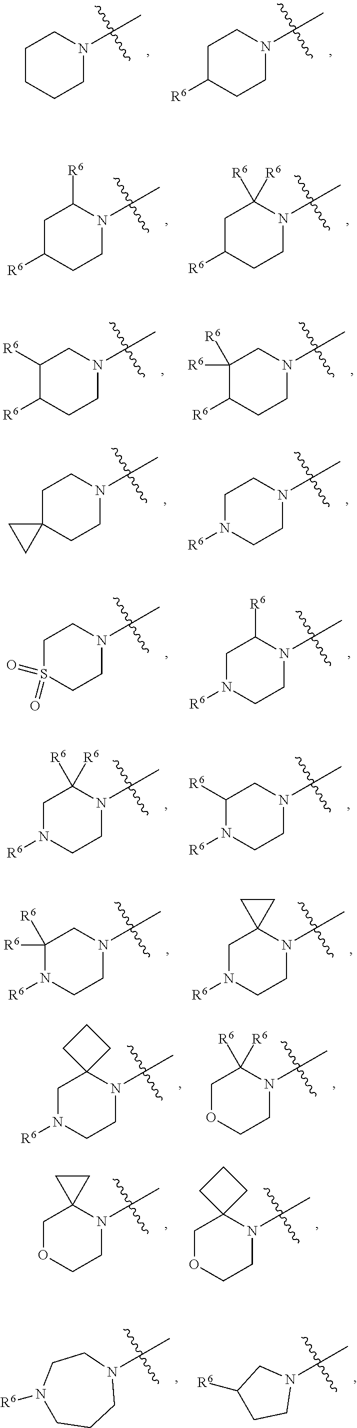

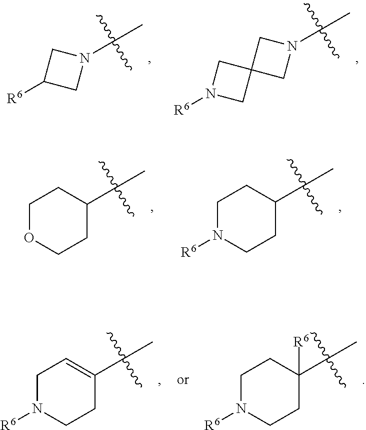

In some embodiments is a compound of Formula (I′), (I), (Ia′), or (II), or a pharmaceutically acceptable salt or solvate thereof, wherein each R⁶ is independently selected from C₁₋₆alkyl, —OR¹⁰, —C(O)OR¹⁰, —N(R¹²)S(O)₂R¹³, —C(O)R¹³, —C(O)N(R¹⁰)(R¹¹), —S(O)₂R¹³, and —S(O)₂N(R¹⁰)(R¹¹)—. In some embodiments is a compound of Formula (I′), (I), (Ia′), or (II), or a pharmaceutically acceptable salt or solvate thereof, wherein R¹ is

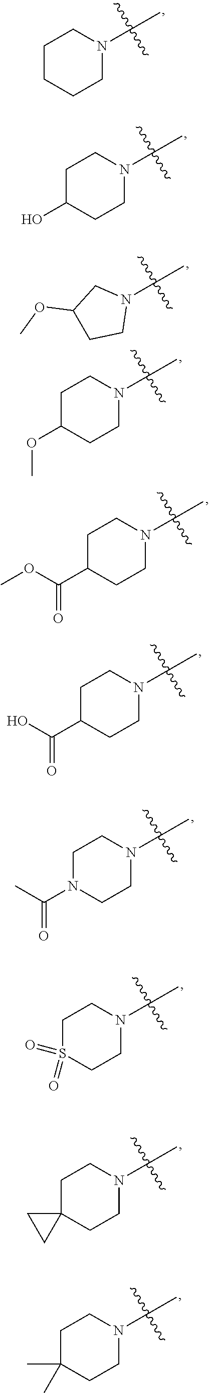

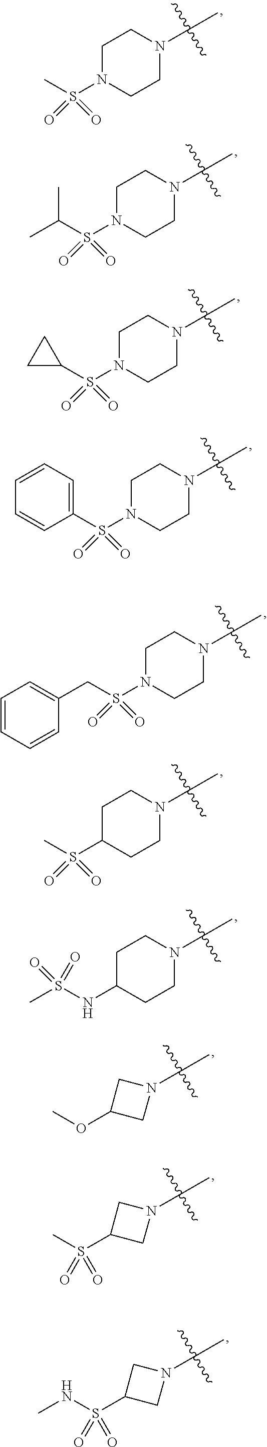

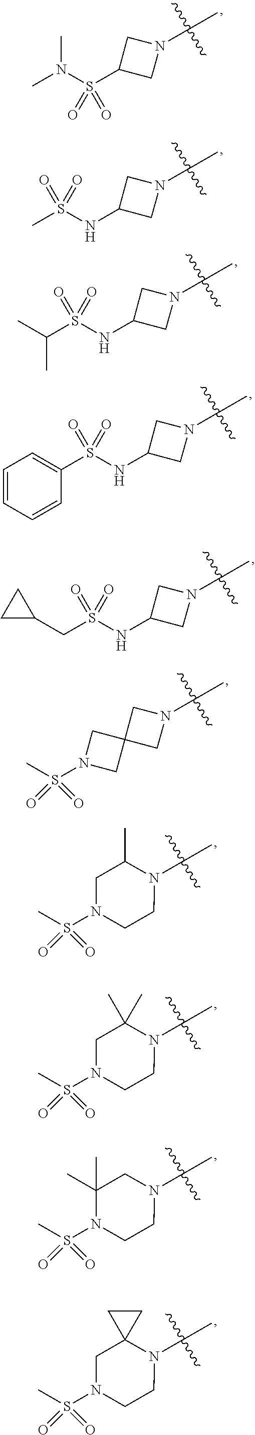

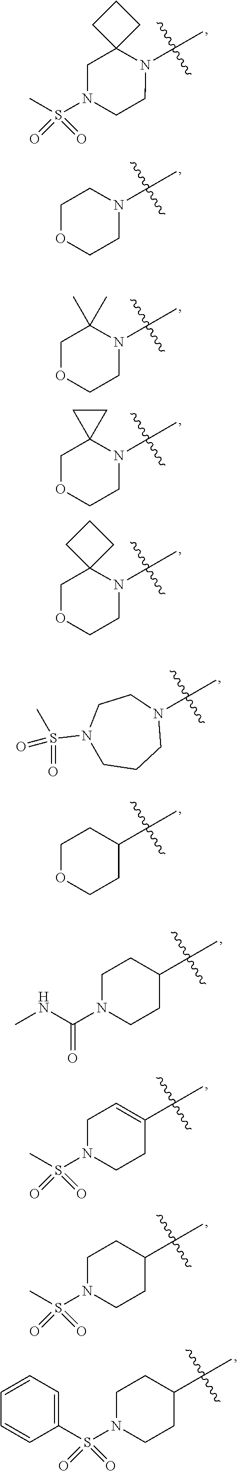

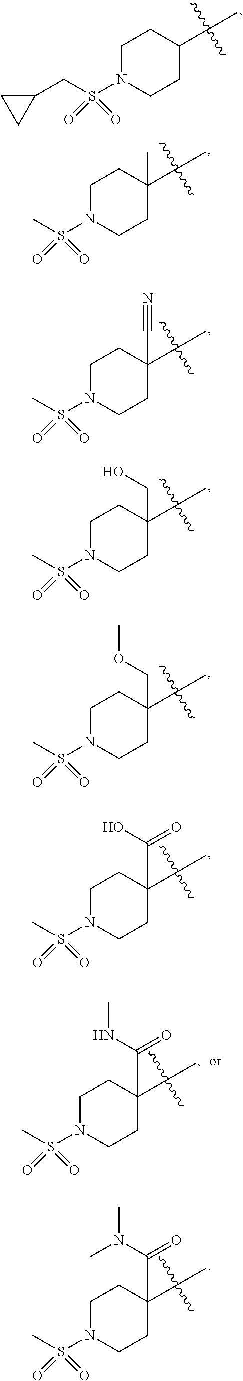

In some embodiments is a compound of Formula (I′), (I), (Ia′), or (II), or a pharmaceutically acceptable salt or solvate thereof, wherein R¹ is

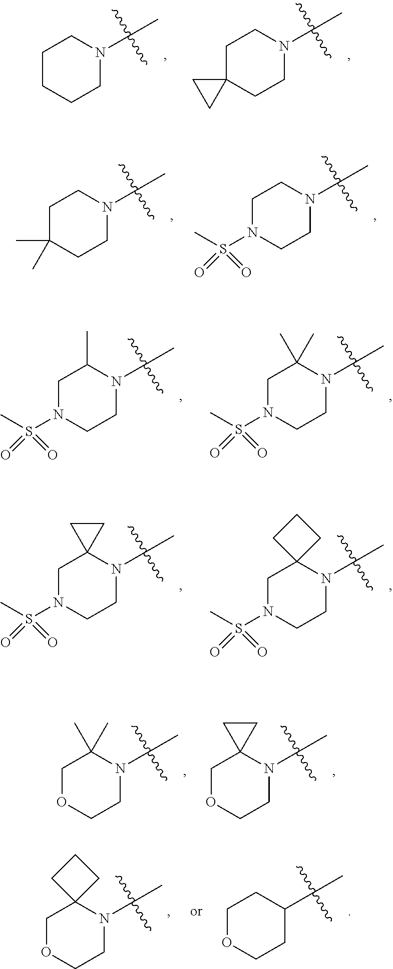

In some embodiments is a compound of Formula (I′), (I), (Ia′), or (II), or a pharmaceutically acceptable salt or solvate thereof, wherein R¹ is C₃₋₈ cycloalkyl optionally substituted with one, two, or three R⁶. In some embodiments is a compound of Formula (I′), (I), (Ia′), or (II), or a pharmaceutically acceptable salt or solvate thereof, wherein R¹ is selected from C₆₋₁₀aryl and C₁₋₉heteroaryl, wherein C₆₋₁₀aryl and C₁₋₉ heteroaryl are substituted with one, two, or three R⁷. In some embodiments is a compound of Formula (I′), (I), (Ia′), or (II), or a pharmaceutically acceptable salt or solvate thereof, wherein R¹ is C₁₋₉heteroaryl substituted with one, two, or three R⁷. In some embodiments is a compound of Formula (I′), (I), (Ia′), or (II), or a pharmaceutically acceptable salt or solvate thereof, wherein R¹ is C₁₋₉heteroaryl selected from pyridinyl, pyrimidinyl, pyrazinyl, pyridazinyl, triazinyl, oxazolyl, thiazolyl, pyrazolyl, furanyl, thienyl, pyrrolyl, imidazolyl, triazolyl, tetrazolyl, isoxazolyl, isothiazolyl, oxadiazolyl, and thiadiazolyl, wherein pyridinyl, pyrimidinyl, pyrazinyl, pyridazinyl, triazinyl, oxazolyl, thiazolyl, pyrazolyl, furanyl, thienyl, pyrrolyl, imidazolyl, triazolyl, tetrazolyl, isoxazolyl, isothiazolyl, oxadiazolyl, and thiadiazolyl are substituted with one, two, or three R⁷. In some embodiments is a compound of Formula (I′), (I), (Ia′), or (II), or a pharmaceutically acceptable salt or solvate thereof, wherein R¹ is

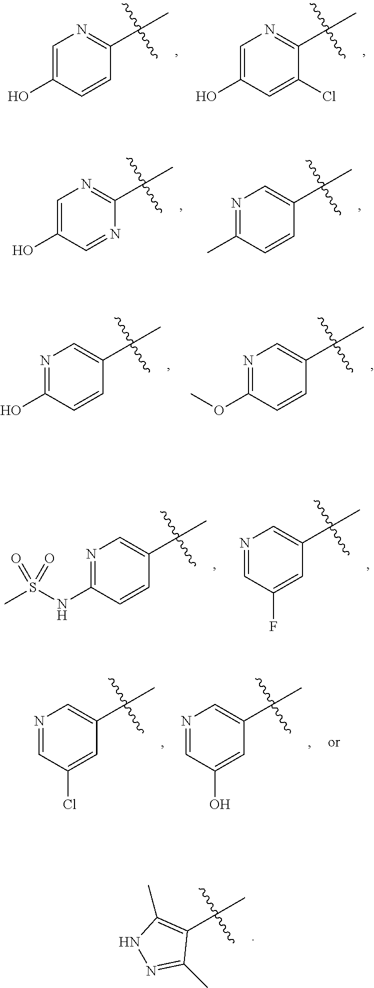

In some embodiments is a compound of Formula (I′), (I), (Ia′), or (II), or a pharmaceutically acceptable salt or solvate thereof, wherein R¹ is phenyl substituted with one, two, or three R⁷. In some embodiments is a compound of Formula (I′), (I), (Ia′), or (II), or a pharmaceutically acceptable salt or solvate thereof, wherein each R⁵ is independently selected from H, halogen, C₁₋₆alkyl, and —OR¹⁰. In some embodiments is a compound of Formula (I′), (I), (Ia′), or (II), or a pharmaceutically acceptable salt or solvate thereof, wherein each R⁵ is H. In some embodiments is a compound of Formula (I′), (I), (Ia′), or (II), or a pharmaceutically acceptable salt or solvate thereof, wherein each R⁴ is independently selected from H, halogen, C₁₋₆alkyl, and C₃₋₆cycloalkyl. In some embodiments is a compound of Formula (I′), (I), (Ia′), or (II), or a pharmaceutically acceptable salt or solvate thereof, wherein each R³ is independently selected from H, halogen, C₁₋₆alkyl, C₁₋₆haloalkyl, and —OR¹⁰. In some embodiments is a compound of Formula (I′), (I), (Ia′), or (II), or a pharmaceutically acceptable salt or solvate thereof, wherein R² is H. In some embodiments is a compound of Formula (I′), (I), (Ia′), or (II), or a pharmaceutically acceptable salt or solvate thereof, wherein R² is halogen.

Any combination of the groups described above for the various variables is contemplated herein. Throughout the specification, groups and substituents thereof are chosen by one skilled in the field to provide stable moieties and compounds.

In one aspect, described herein is a pharmaceutical composition comprising a compound described herein, or a pharmaceutically acceptable salt or solvate thereof, and at least one pharmaceutically acceptable excipient. In some embodiments, the pharmaceutical composition is formulated for administration to a mammal by intravenous administration, subcutaneous administration, oral administration, inhalation, nasal administration, dermal administration, or ophthalmic administration. In some embodiments, the pharmaceutical composition is formulated for administration to a mammal by intravenous administration, subcutaneous administration, or oral administration. In some embodiments, the pharmaceutical composition is formulated for administration to a mammal by oral administration. In some embodiments, the pharmaceutical composition is in the form of a tablet, a pill, a capsule, a liquid, a suspension, a gel, a dispersion, a solution, an emulsion, an ointment, or a lotion. In some embodiments, the pharmaceutical composition is in the form of a tablet, a pill, or a capsule.

In another aspect, described herein is a method of treating or preventing a liver disease or condition in a mammal, comprising administering to the mammal a compound of Formula (I′), (I), (Ia′), or (II), or a pharmaceutically acceptable salt or solvate thereof. In some embodiments, the liver disease or condition is an alcoholic liver disease or condition. In some embodiments, the liver disease or condition is a nonalcoholic liver disease or condition. In some embodiments, the liver disease or condition is liver inflammation, fatty liver (steatosis), liver fibrosis, hepatitis, cirrhosis, hepatocellular carcinoma, or combinations thereof. In some embodiments, the liver disease or condition is primary biliary cirrhosis, primary sclerosing cholangitis, cholestasis, nonalcoholic steatohepatitis (NASH), nonalcoholic fatty liver disease (NAFLD), or combinations thereof.

In another aspect, described herein is a method of treating a disease or condition in a mammal that would benefit from hydroxysteroid 17β-dehydrogenase 13 (HSD17B13) inhibition comprising administering a compound as described herein, or pharmaceutically acceptable salt or solvate thereof, to the mammal in need thereof. In some embodiments, the disease or condition in a mammal that would benefit from HSD17B13 inhibition is liver inflammation, fatty liver (steatosis), liver fibrosis, hepatitis, cirrhosis, hepatocellular carcinoma, or combinations thereof. In some embodiments, the disease or condition in a mammal that would benefit from HSD17B13 inhibition is primary biliary cirrhosis, primary sclerosing cholangitis, cholestasis, nonalcoholic steatohepatitis (NASH), nonalcoholic fatty liver disease (NAFLD), or combinations thereof.

In another aspect, described herein is a method of modulating hydroxysteroid 17p-dehydrogenase 13 (HSD17B13) activity in a mammal, comprising administering to the mammal a compound of Formula (I′), (I), (Ia′), or (II), or a pharmaceutically acceptable salt or solvate thereof. In some embodiments, modulating comprises inhibiting HSD17B13 activity. In some embodiments of a method of modulating HSD17B13 activity in a mammal, the mammal has a liver disease or condition selected from liver inflammation, fatty liver (steatosis), liver fibrosis, hepatitis, cirrhosis, hepatocellular carcinoma, and combinations thereof. In some embodiments of a method of modulating HSD17B13 activity in a mammal, the mammal has a liver disease or condition selected from primary biliary cirrhosis, primary sclerosing cholangitis, cholestasis, nonalcoholic steatohepatitis (NASH), nonalcoholic fatty liver disease (NAFLD), and combinations thereof.

In any of the aforementioned aspects are further embodiments in which the effective amount of the compound described herein, or a pharmaceutically acceptable salt thereof, is: (a) systemically administered to the mammal; and/or (b) administered orally to the mammal; and/or (c) intravenously administered to the mammal; and/or (d) administered by inhalation; and/or (e) administered by nasal administration; or and/or (f) administered by injection to the mammal; and/or (g) administered topically to the mammal; and/or (h) administered by ophthalmic administration; and/or (i) administered rectally to the mammal; and/or (j) administered non-systemically or locally to the mammal.

In any of the embodiments disclosed herein, the mammal or subject is a human.

In some embodiments, compounds provided herein are administered to a human.

In some embodiments, compounds provided herein are orally administered.

Articles of manufacture, which include packaging material, a compound described herein, or a pharmaceutically acceptable salt thereof, within the packaging material, and a label that indicates that the compound or composition, or pharmaceutically acceptable salt, pharmaceutically active metabolite, pharmaceutically acceptable prodrug, or pharmaceutically acceptable solvate thereof, is used for the treatment, prevention or amelioration of one or more symptoms of a disease or condition that would benefit from HSD17B13 inhibition, are provided.

Other objects, features and advantages of the compounds, methods and compositions described herein will become apparent from the following detailed description. It should be understood, however, that the detailed description and the specific examples, while indicating specific embodiments, are given by way of illustration only, since various changes and modifications within the spirit and scope of the instant disclosure will become apparent to those skilled in the art from this detailed description.

### DETAILED DESCRIPTION OF THE INVENTION

Hydroxysteroid dehydrogenase 17013 (HSD17b13) is a member of the short-chain dehydrogenase/reductase enzymes highly expressed in the liver on lipid droplets (Horiguchi et al Biochem Biophysl Res Comm, 2008, 370, 235). It has been shown to oxidize retinol, steroids such as estradiol, and bio-active lipids like leukotriene B4 (Abul-Husn et al NEJM, 2018, 378, 1096 and Ma et al Hepatology, 2019, 69 1504). Exosome sequencing analysis of a large patient population identified a minor allele of HSD17b 13 (rs72613 567:TA) that was associated with reduced odds of developing liver disease (Abul-Husn et al NEJM, 2018, 378, 1096). Relative to subjects with the common HSD17b 13 allele (rs72613567:T), subjects with the TA variant have lower serum ALT and AST and lower odds of alcoholic liver disease with or without cirrhosis, nonalcoholic livers disease with or without cirrhosis, and lower odds of hepatocellular carcinoma. Liver pathology analysis reveals that the subjects with the rs72613567:TA allele have decreased odds of having liver pathology analysis classified as NASH vs normal, NASH vs simple steatosis or NASH with fibrosis vs simple steatosis. Liver injury associated with the PNPLA3 rs738409 (p.I148M) is mitigated by the presence of the rs72613567:TA allele of HSD17b13. Additionally hepatic PNPLA3 mRNA expression is decreased in subjects with the rs72613567:TA allele. The rs72613567:TA allele was found to produce a truncated protein which is unable to metabolize substrates such as estradiol, suggesting the hepatic protective effects of the rs72613567:TA allele is due to loss of enzymatic activity.

Patients with NASH have shown elevated expression of hepatic HSD17b 13 mRNA relative to control subject. Further exploration of the role of HSD17b13 in NASH development identified a minor allele rs62305723 that encodes a P260S mutation of HSD17b13 that leads to loss of retinol metabolism and is associated with decreased hepatic ballooning and inflammation (Ma et al Hepatology, 2019, 69 1504).

HSD17b 13 rs72613567:TA minor allele is associated with loss of HSD17b 13 protein expression in the liver and protection from nonalcoholic steatohepatitis, ballooning degeneration, lobular inflammation and fibrosis. Transcription analysis shows changes in immune-responsive pathways in subjects with rs72613567:TA relative to the major allele (Pirolat et al JLR, 2019, 60, 176).

Subjects with the rs72613567:TA allele of HSD17b 13 are not only found to have lower histological evidence of fibrosis, but decreased hepatic expression of fibrotic genes like TGFb2 and Col3a1. In addition loss of HSD17b13 due to the rs72613567:TA allele has been shown to significantly change the expression of inflammatory gene ALOX5 and decreased plasma IL1b, IL6 and IL-10 (Luukkonen et al, JCI, 2020, 5 e132158). HSD17b13 rs72613567:TA carriers also show increased hepatic phospholipids PC(p16:0/16:0), PE(p16:0/18:1), PC(44:5e), PC(36:2e), PE(34:0), PE(36:3) and PC(34:3) possibly due to decreased phospholipid degradation from a decreased hepatic expression of PLD4.

The HSD17b 13 rs72613567:TA allele, that has been shown to lack HSD17b 13 enzymatic activity, is associated with decreased odds of developing severe fibrosis in patients with chronic HCV infection (About& Abel, NEJM, 2018, 379, 1875). Conversely the major allele rs72613567:T is associated with increasing the risk of development of fibrosis, cirrhosis and HCC in HCV infected patients with the PNPLA3 rs738409:G allele (De Benedittis et al. Gastroenterol Res Pract, 2020, 2020, 4216451).

The loss of function minor allele HSD17b13 rs72613567:TA reduces the risk of developing cirrhosis and hepatocellular carcinoma, is associated with a lower risk of liver-related mortality in the general population and further in patients with cirrhosis (Gellbert-Kristensen et al, Hepatology, 2020, 71, 56). Loss of HSD17b13 function also protects against development of HCC in subjects with alcoholic liver disease (Yang et al, Hepatology, 2019, 70, 231 and Stickel et al, Hepatology, 2020, 72, 88).

PNPLA3 rs738409:G is associated with increased fibrosis in patients with NAFLD. The minor HSD17b13 rs72613567:TA allele has been shown to counteract the PNPLA3 rs738409:G allele and decrease the prevalence of severe inflammation, ballooning and fibrosis (Seko et al, Liver Int, 2020, 40, 1686).

Loss of HSD17b13 enzymatic activity due to carrying the rs72613567:TA allele may delay the onset of autoimmune hepatitis (Mederacke et al, Aliment Pharmacol Ther, 2020, 00, 1).

HSD17b13 rs72613567:TA allele is associated with decreased fibrosis and cirrhosis in patents with copper induced liver injury from Wilson's disease (Ferenci et al, 2019, JHEP, 1, 2).

### Compounds

Compounds described herein, including pharmaceutically acceptable salts, prodrugs, active metabolites and pharmaceutically acceptable solvates thereof, are HSD17B13 inhibitors.

In some embodiments is a compound of Formula (I′), or a pharmaceutically acceptable salt or solvate thereof:

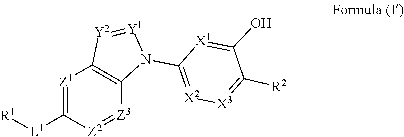

wherein:
X¹, X², and X³ are each independently CR³ or N;
Y¹ and Y² are each independently CR⁴ or N;
Z¹, Z², and Z³ are each independently CR⁵ or N;
L¹ is selected from a bond, —O—, —N(R¹⁰)—, —S(O)₂—, —C(R¹⁰)(R¹¹)N(R¹⁰)—, and —N(R¹⁰)C(R¹⁰)(R¹¹)—;
R¹ is selected from:
   a) C₃₋₈cycloalkyl and C₂₋₉heterocycloalkyl, wherein C₃₋₈cycloalkyl and C₂₋₉heterocycloalkyl are optionally substituted with one, two, or three R⁶; or
   b) C₆₋₁₀aryl and C₁₋₉heteroaryl, wherein C₆₋₁₀aryl and C₁₋₉heteroaryl are substituted with one, two, or three R⁷;
R² is selected from H, halogen, —CN, C₁₋₆alkyl, C₁₋₆haloalkyl, C₂₋₆alkenyl, C₂₋₆alkynyl, C₃₋₆ cycloalkyl, C₂₋₉heterocycloalkyl, C₆₋₁₀aryl, C₁₋₉heteroaryl, —OR¹⁰, —SR¹⁰, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)C(O)N(R¹⁰)(R¹¹), —N(R¹²)C(O)R¹³, —S(O)₂R¹³, —S(O)₂N(R¹⁰)(R¹¹)—, S(═O)(═NH)N(R¹⁰)(R¹¹), —CH₂C(O)N(R¹⁰)(R¹¹), —CH₂N(R¹²)C(O)R¹³, —CH₂S(O)₂R¹³, and —CH₂S(O)₂N(R¹⁰)(R¹¹), wherein C₁₋₆alkyl, C₂₋₆alkenyl, C₂₋₆alkynyl, C₃₋₆ cycloalkyl, C₂₋₉heterocycloalkyl, C₆₋₁₀aryl, and C₁₋₉heteroaryl are optionally substituted with one, two, or three groups selected from halogen, C₁₋₆alkyl, C₁₋₆ haloalkyl, —OR¹⁰, and —N(R¹⁰)(R¹¹);
each R³ and each R⁴ are each independently selected from H, halogen, —CN, C₁₋₆alkyl, C₁₋₆ haloalkyl, C₂₋₆alkenyl, C₂₋₆alkynyl, C₃₋₆cycloalkyl, C₂₋₉heterocycloalkyl, C₁₋₉ heteroaryl, —OR¹⁰, —SR¹⁰, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R¹³, —S(O)₂N(R¹⁰)(R¹¹)—, S(═O)(═NH)N(R¹⁰)(R¹¹), —CH₂C(O)N(R¹⁰)(R¹¹), —CH₂N(R¹²)C(O)R¹³, —CH₂S(O)₂R¹³, and —CH₂S(O)₂N(R¹⁰)(R¹¹), wherein C₁₋₆alkyl, C₂₋₆alkenyl, C₂₋₆alkynyl, C₃₋₆cycloalkyl, C₂₋₉heterocycloalkyl, and C₁₋₉heteroaryl are optionally substituted with one, two, or three groups selected from halogen, C₁₋₆alkyl, C₁₋₆haloalkyl, —OR¹⁰, and —N(R¹⁰)(R¹¹);
each R⁵ is independently selected from H, halogen, —CN, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹⁰, —SR¹⁰, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R¹³, —S(O)₂N(R¹⁰)(R¹¹)—, S(═O)(═NH)N(R¹⁰)(R¹¹), —CH₂C(O)N(R¹⁰)(R¹¹), —CH₂N(R¹²)C(O)R¹³, —CH₂S(O)₂R¹³, and —CH₂S(O)₂N(R¹⁰)(R¹¹), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three groups selected from halogen, C₁₋₆alkyl, C₁₋₆haloalkyl, —OR¹⁰, and —N(R¹⁰)(R¹¹);
each R⁶ and each R⁷ are each independently selected from halogen, oxo, —CN, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹⁰, —SR¹⁰, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R¹³, —S(O)₂N(R¹⁰)(R¹¹)—, S(═O)(═NH)N(R¹⁰)(R¹¹), —CH₂C(O)N(R¹⁰)(R¹¹), —CH₂N(R¹²)C(O)R¹³, —CH₂S(O)₂R¹³, and —CH₂S(O)₂N(R¹⁰)(R¹¹), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three groups selected from halogen, C₁₋₆alkyl, C₁₋₆haloalkyl, —OR¹⁰, —C(O)OR¹⁰, and —N(R¹⁰)(R¹¹);
each R¹⁰ is independently selected from hydrogen, C₁₋₆alkyl, C₁₋₆ haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from halogen, C₁₋₆alkyl, C₁₋₆haloalkyl, C₁₋₆alkoxy, C₃₋₆cycloalkyl, C₂₋₉heterocycloalkyl, C₆₋₁₀aryl, C₁₋₉heteroaryl, —N(R¹¹)(R¹²), and —C(O)OR¹¹;
each R¹¹ is independently selected from hydrogen, C₁₋₆alkyl, and C₁₋₆haloalkyl;
each R¹² is independently selected from hydrogen, C₁₋₆alkyl, and C₁₋₆haloalkyl; and
each R¹³ is independently selected C₁₋₆alkyl, C₁₋₆haloalkyl, C₂₋₆alkenyl, C₂₋₆alkynyl, C₃₋₆ cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three groups selected from halogen, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl.

In some embodiments is a compound of Formula (I), or a pharmaceutically acceptable salt or solvate thereof:

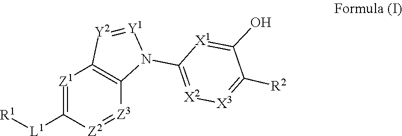

wherein:
X¹, X², and X³ are each independently CR³ or N;
Y¹ and Y² are each independently CR⁴ or N;
Z¹, Z², and Z³ are each independently CR⁵ or N;
L¹ is selected from a bond, —O—, —N(R¹⁰)—, —S(O)₂—, —C(R¹⁰)(R¹¹)N(R¹⁰)—, and —N(R¹⁰)C(R¹⁰)(R¹¹)—;
R¹ is selected from:
   a) C₃₋₈cycloalkyl and C₂₋₉heterocycloalkyl, wherein C₃₋₈cycloalkyl and C₂₋₉heterocycloalkyl are optionally substituted with one, two, or three R⁶; or
   b) C₆₋₁₀aryl and C₁₋₉heteroaryl, wherein C₆₋₁₀aryl and C₁₋₉heteroaryl are substituted with one, two, or three R⁷;
R² is selected from H, halogen, —CN, C₁₋₆alkyl, C₁₋₆haloalkyl, C₂₋₆alkenyl, C₂₋₆alkynyl, C₃₋₆ cycloalkyl, C₂₋₉heterocycloalkyl, C₆₋₁₀aryl, C₁₋₉heteroaryl, —OR¹⁰, —SR¹⁰, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R¹³, —S(O)₂N(R¹⁰)(R¹¹)—, S(═O)(═NH)N(R¹⁰)(R¹¹), —CH₂C(O)N(R¹⁰)(R¹¹), —CH₂N(R¹²)C(O)R¹³, —CH₂S(O)₂R¹³, and —CH₂S(O)₂N(R¹⁰)(R¹¹), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three groups selected from halogen, C₁₋₆alkyl, C₁₋₆haloalkyl, —OR¹⁰, and —N(R¹⁰)(R¹¹);
each R³, each R⁴, and each R⁵ are each independently selected from H, halogen, —CN, C₁₋₆alkyl, —C₁₋₆haloalkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹⁰, —SR¹⁰, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R¹³, —S(O)₂N(R¹⁰)(R¹¹)—, S(═O)(═NH)N(R¹⁰)(R¹¹), —CH₂C(O)N(R¹⁰)(R¹¹), —CH₂N(R¹²)C(O)R¹³, —CH₂S(O)₂R¹³, and —CH₂S(O)₂N(R¹⁰)(R¹¹), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three groups selected from halogen, C₁₋₆alkyl, C₁₋₆haloalkyl, —OR¹⁰, and —N(R¹⁰)(R¹¹);
each R⁶ and each R⁷ are each independently selected from halogen, oxo, —CN, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹⁰, —SR¹⁰, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R¹³, —S(O)₂N(R¹⁰)(R¹¹)—, S(═O)(═NH)N(R¹⁰)(R¹¹), —CH₂C(O)N(R¹⁰)(R¹¹), —CH₂N(R¹²)C(O)R¹³, —CH₂S(O)₂R¹³, and —CH₂S(O)₂N(R¹⁰)(R¹¹), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three groups selected from halogen, C₁₋₆alkyl, C₁₋₆haloalkyl, —OR¹⁰, and —N(R¹⁰)(R¹¹);
each R¹⁰ is independently selected from hydrogen, C₁₋₆alkyl, C₁₋₆ haloalkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from halogen, C₁₋₆alkyl, C₁₋₆haloalkyl, C₁₋₆alkoxy, C₃₋₆ cycloalkyl, C₂₋₉heterocycloalkyl, C₆₋₁₀aryl, and C₁₋₉heteroaryl;
each R¹¹ is independently selected from hydrogen, C₁₋₆alkyl, and C₁₋₆haloalkyl;
each R¹² is independently selected from hydrogen, C₁₋₆alkyl, and C₁₋₆haloalkyl; and
each R¹³ is independently selected C₁₋₆alkyl, C₁₋₆haloalkyl, C₂₋₆alkenyl, C₂₋₆alkynyl, C₃₋₆ cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three groups selected from halogen, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl.

In some embodiments is a compound of Formula (I′) or (I), or a pharmaceutically acceptable salt or solvate thereof, wherein X¹, X², and X³ are each CR³. In some embodiments is a compound of Formula (I′) or (I), or a pharmaceutically acceptable salt or solvate thereof, wherein X¹, X², and X³ are each CR³ and each R³ is independently selected from H, halogen, C₁₋₆alkyl, C₁₋₆haloalkyl, and —OR¹⁰. In some embodiments is a compound of Formula (I′) or (I), or a pharmaceutically acceptable salt or solvate thereof, wherein X¹, X², and X³ are each CR³ and each R³ is independently selected from H, halogen, C₁₋₆alkyl, and C₁₋₆haloalkyl. In some embodiments is a compound of Formula (I′) or (I), or a pharmaceutically acceptable salt or solvate thereof, wherein X¹, X², and X³ are each CR³ and each R³ is independently selected from H, halogen, and C₁₋₆haloalkyl. In some embodiments is a compound of Formula (I′) or (I), or a pharmaceutically acceptable salt or solvate thereof, wherein X¹ is C(H), X² is C(H), and X³ is C(CF₃). In some embodiments is a compound of Formula (I′) or (I), or a pharmaceutically acceptable salt or solvate thereof, wherein X¹ is C(F), X² is C(H), and X³ is C(CF₃). In some embodiments is a compound of Formula (I′) or (I), or a pharmaceutically acceptable salt or solvate thereof, wherein X¹ is C(Cl), X² is C(H), and X³ is C(CF₃). In some embodiments is a compound of Formula (I′) or (I), or a pharmaceutically acceptable salt or solvate thereof, wherein X¹ is C(H), X² is C(H), and X³ is C(F). In some embodiments is a compound of Formula (I′) or (I), or a pharmaceutically acceptable salt or solvate thereof, wherein X¹ is C(H), X² is C(H), and X³ is C(Cl).

In some embodiments is a compound of Formula (I′) or (I), or a pharmaceutically acceptable salt or solvate thereof, wherein R² is selected from H, halogen, C₁₋₆alkyl, C₁₋₆ haloalkyl, and —OR¹⁰. In some embodiments is a compound of Formula (I′) or (I), or a pharmaceutically acceptable salt or solvate thereof, wherein R² is H. In some embodiments is a compound of Formula (I′) or (I), or a pharmaceutically acceptable salt or solvate thereof, wherein R² is halogen. In some embodiments is a compound of Formula (I′) or (I), or a pharmaceutically acceptable salt or solvate thereof, wherein R² is C₁₋₆alkyl. In some embodiments is a compound of Formula (I′) or (I), or a pharmaceutically acceptable salt or solvate thereof, wherein R² is C₁₋₆haloalkyl. In some embodiments is a compound of Formula (I′) or (I), or a pharmaceutically acceptable salt or solvate thereof, wherein R² is —OR¹⁰. In some embodiments is a compound of Formula (I′) or (I), or a pharmaceutically acceptable salt or solvate thereof, wherein R² is —OH. In some embodiments is a compound of Formula (I′) or (I), or a pharmaceutically acceptable salt or solvate thereof, wherein R² is —OCH₃.

In some embodiments is a compound of Formula (I′) or (I), or a pharmaceutically acceptable salt or solvate thereof, wherein Y¹ is N and Y² is CR⁴. In some embodiments is a compound of Formula (I′) or (I), or a pharmaceutically acceptable salt or solvate thereof, wherein Y¹ is CR⁴ and Y² is CR⁴. In some embodiments is a compound of Formula (I′) or (I), or a pharmaceutically acceptable salt or solvate thereof, wherein Y¹ is CR⁴ and Y² is N. In some embodiments is a compound of Formula (I′) or (I), or a pharmaceutically acceptable salt or solvate thereof, wherein each R⁴ is independently selected from H, halogen, C₁₋₆alkyl, and C₃₋₆cycloalkyl. In some embodiments is a compound of Formula (I′) or (I), or a pharmaceutically acceptable salt or solvate thereof, wherein Y¹ is N and Y² is C(H). In some embodiments is a compound of Formula (I′) or (I), or a pharmaceutically acceptable salt or solvate thereof, wherein Y¹ is C(H) and Y² is C(H). In some embodiments is a compound of Formula (I′) or (I), or a pharmaceutically acceptable salt or solvate thereof, wherein Y¹ is C(H) and Y² is N. In some embodiments is a compound of Formula (I′) or (I), or a pharmaceutically acceptable salt or solvate thereof, wherein Y¹ is N and Y² is N.

In some embodiments is a compound of Formula (I′) or (I), or a pharmaceutically acceptable salt or solvate thereof, wherein Z¹, Z², and Z³ are CR⁵. In some embodiments is a compound of Formula (I′) or (I), or a pharmaceutically acceptable salt or solvate thereof, wherein Z¹ is N; and Z² and Z³ are CR⁵. In some embodiments is a compound of Formula (I′) or (I), or a pharmaceutically acceptable salt or solvate thereof, wherein Z² is N; and Z¹ and Z³ are CR⁵. In some embodiments is a compound of Formula (I′) or (I), or a pharmaceutically acceptable salt or solvate thereof, wherein Z³ is N; and Z¹ and Z² are CR⁵. In some embodiments is a compound of Formula (I′) or (I), or a pharmaceutically acceptable salt or solvate thereof, wherein Z¹ is CR⁵; and Z² and Z³ are N. In some embodiments is a compound of Formula (I′) or (I), or a pharmaceutically acceptable salt or solvate thereof, wherein Z² is CR⁵; and Z¹ and Z³ are N. In some embodiments is a compound of Formula (I′) or (I), or a pharmaceutically acceptable salt or solvate thereof, wherein Z³ is CR⁵; and Z¹ and Z² are N. In some embodiments is a compound of Formula (I′) or (I), or a pharmaceutically acceptable salt or solvate thereof, wherein each R⁵ is independently selected from H, halogen, C₁₋₆alkyl, and —OR¹⁰. In some embodiments is a compound of Formula (I′) or (I), or a pharmaceutically acceptable salt or solvate thereof, wherein each R⁵ is H. In some embodiments is a compound of Formula (I′) or (I), or a pharmaceutically acceptable salt or solvate thereof, wherein Z¹, Z², and Z³ are C(H). In some embodiments is a compound of Formula (I′) or (I), or a pharmaceutically acceptable salt or solvate thereof, wherein Z¹ is N; and Z² and Z³ are C(H). In some embodiments is a compound of Formula (I′) or (I), or a pharmaceutically acceptable salt or solvate thereof, wherein Z² is N; and Z¹ and Z³ are C(H). In some embodiments is a compound of Formula (I′) or (I), or a pharmaceutically acceptable salt or solvate thereof, wherein Z³ is N; and Z¹ and Z² are C(H). In some embodiments is a compound of Formula (I′) or (I), or a pharmaceutically acceptable salt or solvate thereof, wherein Z¹ is C(H); and Z² and Z³ are N. In some embodiments is a compound of Formula (I′) or (I), or a pharmaceutically acceptable salt or solvate thereof, wherein Z² is C(H); and Z¹ and Z³ are N. In some embodiments is a compound of Formula (I) or (I), or a pharmaceutically acceptable salt or solvate thereof, wherein Z³ is C(H); and Z^{L} and Z² are N.

In some embodiments is a compound of Formula (I′) or (I), or a pharmaceutically acceptable salt or solvate thereof, wherein L¹ is a bond. In some embodiments is a compound of Formula (I′) or (I), or a pharmaceutically acceptable salt or solvate thereof, wherein L¹ is —O—. In some embodiments is a compound of Formula (I) or (I), or a pharmaceutically acceptable salt or solvate thereof, wherein L¹ is —N(R¹⁰)—. In some embodiments is a compound of Formula (I′) or (I), or a pharmaceutically acceptable salt or solvate thereof, wherein L¹ is —N(H)—. In some embodiments is a compound of Formula (I) or (I), or a pharmaceutically acceptable salt or solvate thereof, wherein L¹ is —N(CH₃)—. In some embodiments is a compound of Formula (I′) or (I), or a pharmaceutically acceptable salt or solvate thereof, wherein L¹ is —C(R¹⁰)(R¹¹)N(R¹⁰)—. In some embodiments is a compound of Formula (I′) or (I), or a pharmaceutically acceptable salt or solvate thereof, wherein L¹ is —CH₂N(H)—. In some embodiments is a compound of Formula (I) or (I), or a pharmaceutically acceptable salt or solvate thereof, wherein L¹ is —N(R¹⁰)C(R¹⁰)(R¹¹)—. In some embodiments is a compound of Formula (I′) or (I), or a pharmaceutically acceptable salt or solvate thereof, wherein L¹ is —N(H)CH₂—.

In some embodiments is a compound of Formula (I′) or (I), or a pharmaceutically acceptable salt or solvate thereof, wherein R¹ is selected from C₃₋₈cycloalkyl and C₂₋₉heterocycloalkyl, wherein C₃₋₈cycloalkyl and C₂₋₉heterocycloalkyl are optionally substituted with one, two, or three R⁶. In some embodiments is a compound of Formula (I′) or (I), or a pharmaceutically acceptable salt or solvate thereof, wherein R¹ is C₂₋₉heterocycloalkyl optionally substituted with one, two, or three R⁶. In some embodiments is a compound of Formula (I′) or (I), or a pharmaceutically acceptable salt or solvate thereof, wherein R¹ is C₂₋₉heterocycloalkyl selected from piperidinyl, piperazinyl, morpholinyl, tetrahydropyranyl, tetrahydrofuranyl, pyrrolidinyl, oxetanyl, azetidinyl, aziridinyl, azepanyl, diazepanyl, 6-azaspiro[2.5]octanyl, 4,7-diazaspiro[2.5]octanyl, 7-oxa-4-azaspiro[2.5]octanyl, 5,8-diazaspiro[3.5]nonanyl, 8-oxa-5-azaspiro[3.5]nonanyl, or 2,6-diazaspiro[3.3]heptanyl, wherein piperidinyl, piperazinyl, morpholinyl, tetrahydropyranyl, tetrahydrofuranyl, pyrrolidinyl, oxetanyl, azetidinyl, aziridinyl, azepanyl, diazepanyl, 6-azaspiro[2.5]octanyl, 4,7-diazaspiro[2.5]octanyl, 7-oxa-4-azaspiro[2.5]octanyl, 5,8-diazaspiro[3.5]nonanyl, 8-oxa-5-azaspiro[3.5]nonanyl, or 2,6-diazaspiro[3.3]heptanyl are optionally substituted with one, two, or three R⁶. In some embodiments is a compound of Formula (I′) or (I), or a pharmaceutically acceptable salt or solvate thereof, wherein R¹ is

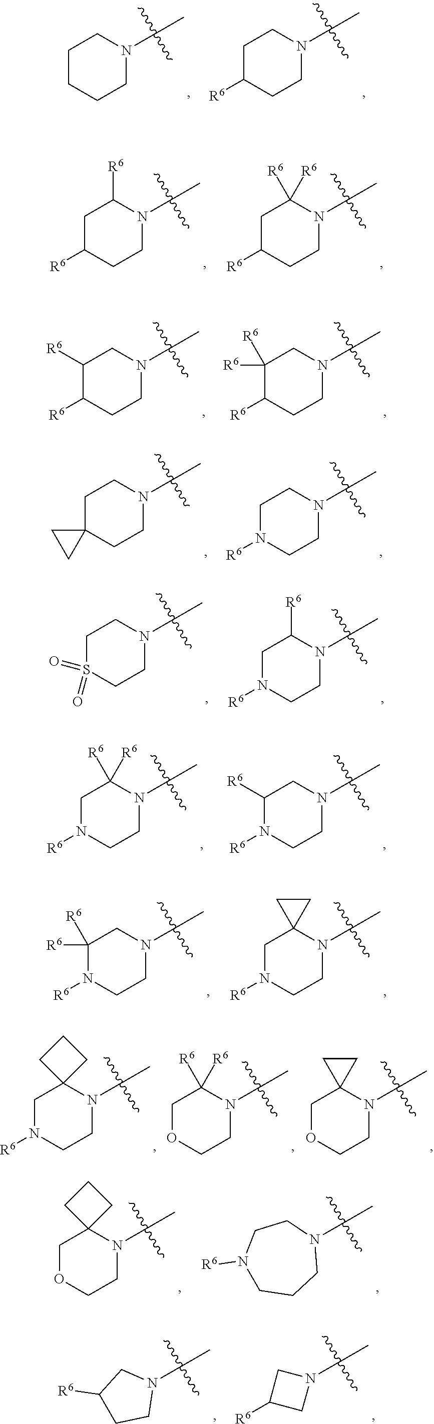

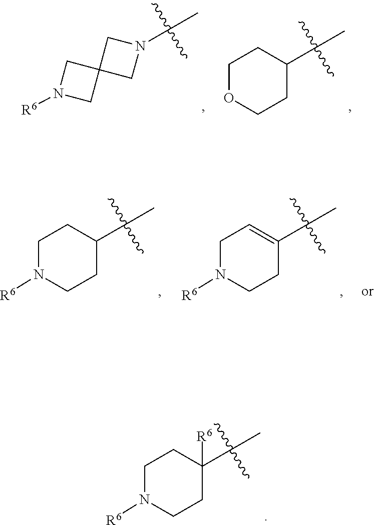

In some embodiments is a compound of Formula (I′) or (I), or a pharmaceutically acceptable salt or solvate thereof, wherein each R⁶ is independently selected from C₁₋₆alkyl, —OR¹⁰, —C(O)OR¹⁰, —N(R¹²)S(O)₂R¹³, —C(O)R¹³, —C(O)N(R¹⁰)(R¹¹), —S(O)₂R¹³, and —S(O)₂N(R¹⁰)(R¹¹)—. In some embodiments is a compound of Formula (I′) or (I), or a pharmaceutically acceptable salt or solvate thereof, wherein R is

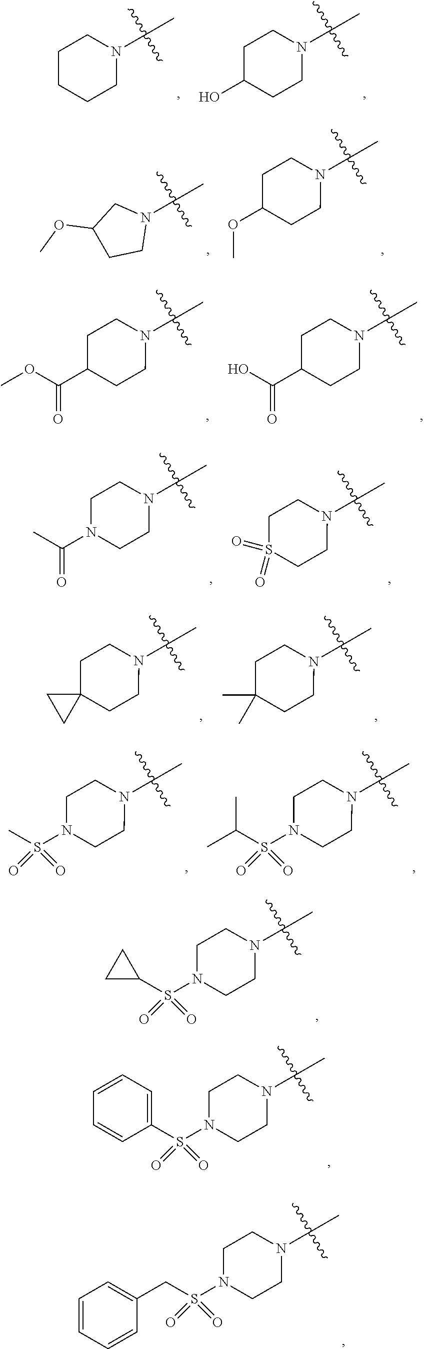

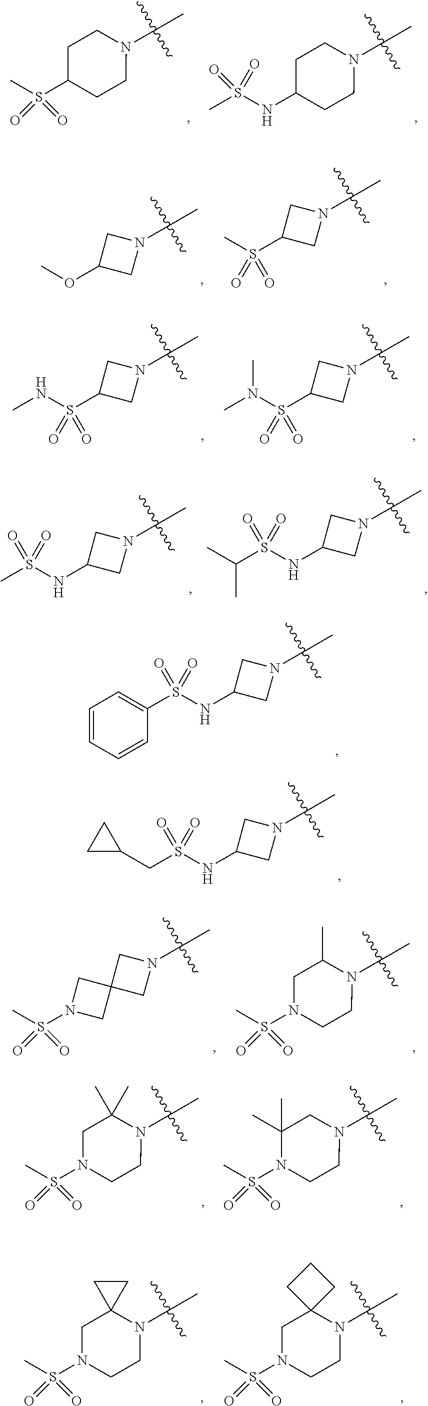

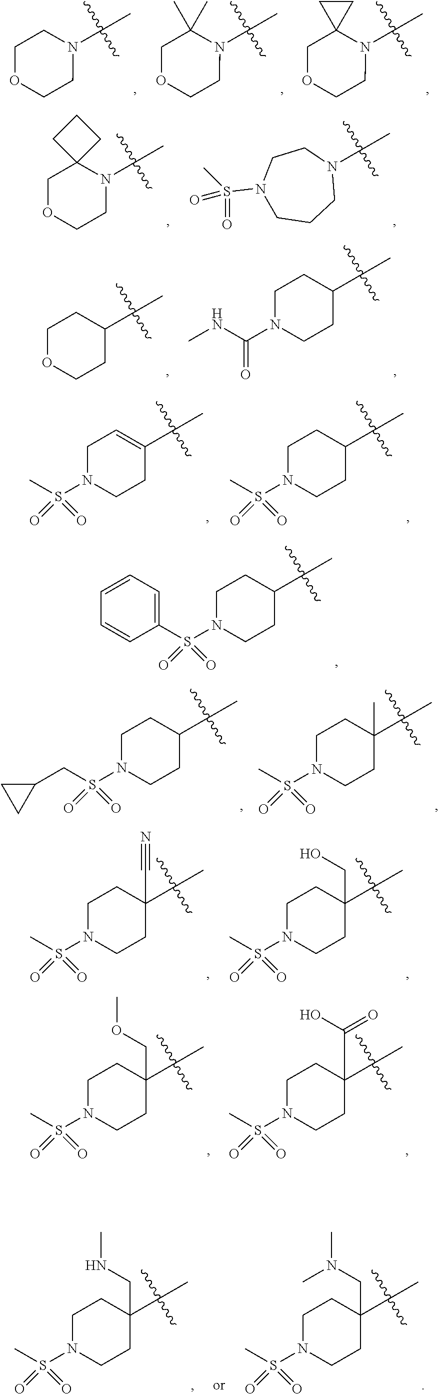

In some embodiments is a compound of Formula (I′) or (I), or a pharmaceutically acceptable salt or solvate thereof, wherein R¹ is

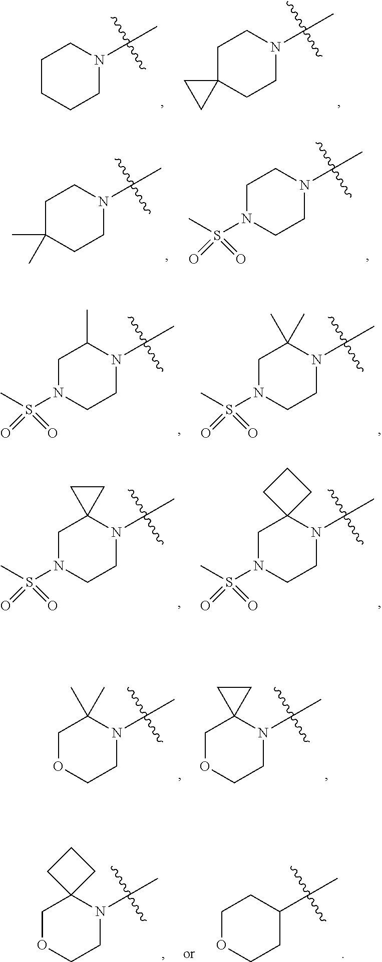

In some embodiments is a compound of Formula (I′) or (I), or a pharmaceutically acceptable salt or solvate thereof, wherein R¹ is

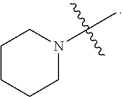 [SMILES: *N1CCCCC1]

In some embodiments is a compound of Formula (I′) or (I), or a pharmaceutically acceptable salt or solvate thereof, wherein R¹ is

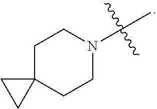 [SMILES: *N1CCC2(CC1)CC2]

In some embodiments is a compound of Formula (I′) or (I), or a pharmaceutically acceptable salt or solvate thereof, wherein R¹ is

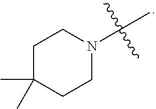 [SMILES: *N1CCC(C)(C)CC1]

In some embodiments is a compound of Formula (I′) or (I), or a pharmaceutically acceptable salt or solvate thereof, wherein R¹ is

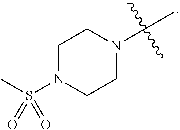 [SMILES: *N1CCN(S(C)(=O)=O)CC1]

In some embodiments is a compound of Formula (I′) or (I), or a pharmaceutically acceptable salt or solvate thereof, wherein R¹ is

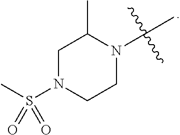 [SMILES: *N1CCN(S(C)(=O)=O)CC1C]

In some embodiments is a compound of Formula (I′) or (I), or a pharmaceutically acceptable salt or solvate thereof, wherein R¹ is

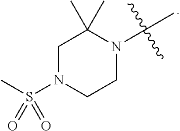 [SMILES: *N1CCN(S(C)(=O)=O)CC1(C)C]

In some embodiments is a compound of Formula (I′) or (I), or a pharmaceutically acceptable salt or solvate thereof, wherein R¹ is

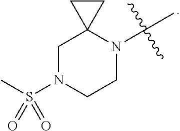 [SMILES: *N1CCN(S(C)(=O)=O)CC12CC2]

In some embodiments is a compound of Formula (I′) or (I), or a pharmaceutically acceptable salt or solvate thereof, wherein R¹ is

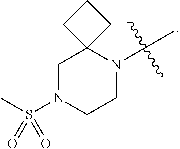 [SMILES: *N1CCN(S(C)(=O)=O)CC12CCC2]

In some embodiments is a compound of Formula (I′) or (I), or a pharmaceutically acceptable salt or solvate thereof, wherein R¹ is

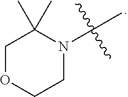 [SMILES: *N1CCOCC1(C)C]

In some embodiments is a compound of Formula (I′) or (I), or a pharmaceutically acceptable salt or solvate thereof, wherein R¹ is

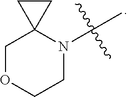 [SMILES: *N1CCOCC12CC2]

In some embodiments is a compound of Formula (I′) or (I), or a pharmaceutically acceptable salt or solvate thereof, wherein R¹ is

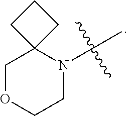 [SMILES: *N1CCOCC12CCC2]

In some embodiments is a compound of Formula (I′) or (I), or a pharmaceutically acceptable salt or solvate thereof, wherein R¹ is

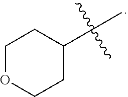 [SMILES: *C1CCOCC1]

In some embodiments is a compound of Formula (I′) or (I), or a pharmaceutically acceptable salt or solvate thereof, wherein R¹ is C₃₋₈cycloalkyl optionally substituted with one, two, or three R⁶.

In some embodiments is a compound of Formula (I′) or (I), or a pharmaceutically acceptable salt or solvate thereof, wherein R¹ is selected from C₆₋₁₀aryl and C₁₋₉heteroaryl, wherein C₆₋₁₀aryl and C₁₋₉heteroaryl are substituted with one, two, or three R⁷. In some embodiments is a compound of Formula (I′) or (I), or a pharmaceutically acceptable salt or solvate thereof, wherein R¹ is C₁₋₉heteroaryl substituted with one, two, or three R⁷. In some embodiments is a compound of Formula (I′) or (I), or a pharmaceutically acceptable salt or solvate thereof, wherein R¹ is C₁₋₉heteroaryl selected from pyridinyl, pyrimidinyl, pyrazinyl, pyridazinyl, triazinyl, oxazolyl, thiazolyl, pyrazolyl, furanyl, thienyl, pyrrolyl, imidazolyl, triazolyl, tetrazolyl, isoxazolyl, isothiazolyl, oxadiazolyl, and thiadiazolyl, wherein pyridinyl, pyrimidinyl, pyrazinyl, pyridazinyl, triazinyl, oxazolyl, thiazolyl, pyrazolyl, furanyl, thienyl, pyrrolyl, imidazolyl, triazolyl, tetrazolyl, isoxazolyl, isothiazolyl, oxadiazolyl, and thiadiazolyl are substituted with one, two, or three R⁷. In some embodiments is a compound of Formula (I′) or (I), or a pharmaceutically acceptable salt or solvate thereof, wherein R¹ is

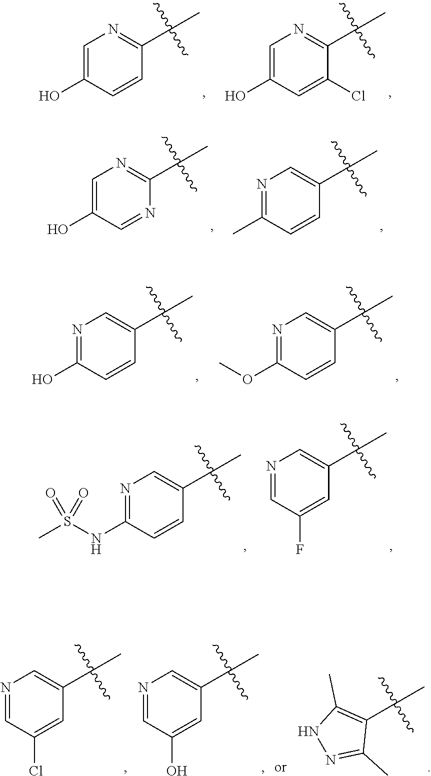

In some embodiments is a compound of Formula (I′) or (I), or a pharmaceutically acceptable salt or solvate thereof, wherein R¹ is phenyl substituted with one, two, or three R⁷.

In some embodiments is a compound of Formula (Ia′), or a pharmaceutically acceptable salt or solvate thereof:

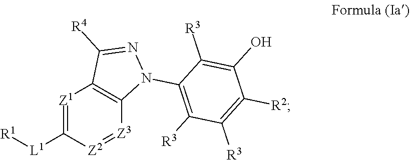

wherein:
Z¹, Z², and Z³ are each independently CR⁵ or N;
L¹ is selected from a bond, —O—, —N(R¹⁰)—, —S(O)₂—, —C(R¹⁰)(R¹¹)N(R¹⁰)—, and —N(R¹⁰)C(R¹⁰)(R¹¹);
R¹ is selected from:
   a) C₃₋₈cycloalkyl and C₂₋₉heterocycloalkyl, wherein C₃₋₈cycloalkyl and C₂₋₉heterocycloalkyl are optionally substituted with one, two, or three R⁶; or
   b) C₆₋₁₀aryl and C₁₋₉heteroaryl, wherein C₆₋₁₀aryl and C₁₋₉heteroaryl are substituted with one, two, or three R⁷;
R² is selected from H, halogen, —CN, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹⁰, —SR¹⁰, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)C(O)N(R¹⁰)(R¹¹), —N(R¹²)C(O)R¹³, —S(O)₂R¹³, —S(O)₂N(R¹⁰)(R¹¹)—, S(═O)(═NH)N(R¹⁰)(R¹¹), —CH₂C(O)N(R¹⁰)(R¹¹), —CH₂N(R¹²)C(O)R¹³, —CH₂S(O)₂R¹³, and —CH₂S(O)₂N(R¹⁰)(R¹¹), wherein C₁₋₆alkyl, C₂₋₆alkenyl, C₂₋₆alkynyl, C₃₋₆ cycloalkyl, C₂₋₉heterocycloalkyl, C₆₋₁₀aryl, and C₁₋₉heteroaryl are optionally substituted with one, two, or three groups selected from halogen, C₁₋₆alkyl, C₁₋₆ haloalkyl, —OR¹⁰, and —N(R¹⁰)(R¹¹);
each R³ and R⁴ are each independently selected from H, halogen, —CN, C₁₋₆alkyl, C₁₋₆ haloalkyl, C₂₋₆alkenyl, C₂₋₆alkynyl, C₃₋₆cycloalkyl, C₂₋₉heterocycloalkyl, C₁₋₉ heteroaryl, —OR¹⁰, —SR¹⁰, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R¹³, —S(O)₂N(R¹⁰)(R¹¹)—, S(═O)(═NH)N(R¹⁰)(R¹¹), —CH₂C(O)N(R¹⁰)(R¹¹), —CH₂N(R¹²)C(O)R¹³, —CH₂S(O)₂R¹³, and —CH₂S(O)₂N(R¹⁰)(R¹¹), wherein C₁₋₆alkyl, C₂₋₆alkenyl, C₂₋₆alkynyl, C₃₋₆cycloalkyl, C₂₋₉heterocycloalkyl, and C₁₋₉heteroaryl are optionally substituted with one, two, or three groups selected from halogen, C₁₋₆alkyl, C₁₋₆haloalkyl, —OR¹⁰, and —N(R¹⁰)(R¹¹);
each R⁵ is independently selected from H, halogen, —CN, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹⁰, —SR¹⁰, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R¹³, —S(O)₂N(R¹⁰)(R⁰¹)—, S(═O)(═NH)N(R¹⁰)(R¹¹), —CH₂C(O)N(R¹⁰)(R¹¹), —CH₂N(R¹²)C(O)R¹³, —CH₂S(O)₂R¹³, and —CH₂S(O)₂N(R¹⁰)(R¹¹), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three groups selected from halogen, C₁₋₆alkyl, C₁₋₆haloalkyl, —OR¹⁰, and —N(R¹⁰)(R¹¹);
each R⁶ and each R⁷ are each independently selected from halogen, oxo, —CN, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹⁰, —SR¹⁰, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R¹³, —S(O)₂N(R¹⁰)(R¹¹)—, S(═O)(═NH)N(R¹⁰)(R¹¹), —CH₂C(O)N(R¹⁰)(R¹¹), —CH₂N(R¹²)C(O)R¹³, —CH₂S(O)₂R¹³, and —CH₂S(O)₂N(R¹⁰)(R¹¹), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three groups selected from halogen, C₁₋₆alkyl, C₁₋₆haloalkyl, —OR¹⁰, —C(O)OR¹⁰, and —N(R¹⁰)(R¹¹);
each R¹⁰ is independently selected from hydrogen, C₁₋₆alkyl, C₁₋₆ haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from halogen, C₁₋₆alkyl, C₁₋₆haloalkyl, C₁₋₆alkoxy, C₃₋₆cycloalkyl, C₂₋₉heterocycloalkyl, C₆₋₁₀aryl, C₁₋₉heteroaryl, —N(R¹¹)(R¹²), and —C(O)OR¹¹;
each R¹¹ is independently selected from hydrogen, C₁₋₆alkyl, and C₁₋₆haloalkyl;
each R¹² is independently selected from hydrogen, C₁₋₆alkyl, and C₁₋₆haloalkyl; and
each R¹³ is independently selected C₁₋₆alkyl, C₁₋₆haloalkyl, C₂₋₆alkenyl, C₂₋₆alkynyl, C₃₋₆ cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three groups selected from halogen, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl.

In some embodiments is a compound of Formula (Ia), or a pharmaceutically acceptable salt or solvate thereof:

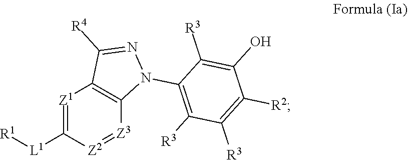

wherein:
Z¹, Z², and Z³ are each independently CR⁵ or N;
L¹ is selected from a bond, —O—, —N(R¹⁰)—, —S(O)₂—, —C(R¹⁰)(R¹¹)N(R¹⁰)—, and —N(R¹⁰)C(R¹⁰)(R¹¹)—;
R¹ is selected from:
   a) C₃₋₈cycloalkyl and C₂₋₉heterocycloalkyl, wherein C₃₋₈cycloalkyl and C₂₋₉heterocycloalkyl are optionally substituted with one, two, or three R⁶; or
   b) C₆₋₁₀aryl and C₁₋₉heteroaryl, wherein C₆₋₁₀aryl and C₁₋₉heteroaryl are substituted with one, two, or three R⁷;
R² is selected from H, halogen, —CN, C₁₋₆alkyl, C₁₋₆haloalkyl, C₂₋₆alkenyl, C₂₋₆alkynyl, C₃₋₆ cycloalkyl, C₂₋₉heterocycloalkyl, C₆₋₁₀aryl, C₁₋₉heteroaryl, —OR¹⁰, —SR¹⁰, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R¹³, —S(O)₂N(R¹⁰)(R¹¹)—, S(═O)(═NH)N(R¹⁰)(R¹¹), —CH₂C(O)N(R¹⁰)(R¹¹), —CH₂N(R¹²)C(O)R¹³, —CH₂S(O)₂R¹³, and —CH₂S(O)₂N(R¹⁰)(R¹¹), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three groups selected from halogen, C₁₋₆alkyl, C₁₋₆haloalkyl, —OR¹⁰, and —N(R¹⁰)(R¹¹);
each R³, R⁴, and each R⁵ are each independently selected from H, halogen, —CN, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹⁰, —SR¹⁰, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R¹³, —S(O)₂N(R¹⁰)(R¹¹)—, S(═O)(═NH)N(R¹⁰)(R¹¹), —CH₂C(O)N(R¹⁰)(R¹¹), —CH₂N(R¹²)C(O)R¹³, —CH₂S(O)₂R¹³, and —CH₂S(O)₂N(R¹⁰)(R¹¹), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three groups selected from halogen, C₁₋₆alkyl, C₁₋₆haloalkyl, —OR¹⁰, and —N(R¹⁰)(R¹¹);
each R⁶ and each R⁷ are each independently selected from halogen, oxo, —CN, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹⁰, —SR¹⁰, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R¹³, —S(O)₂N(R¹⁰)(R¹¹)—, S(═O)(═NH)N(R¹⁰)(R¹¹), —CH₂C(O)N(R¹⁰)(R¹¹), —CH₂N(R¹²)C(O)R¹³, —CH₂S(O)₂R¹³, and —CH₂S(O)₂N(R¹⁰)(R¹¹), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three groups selected from halogen, C₁₋₆alkyl, C₁₋₆haloalkyl, —OR¹⁰, and —N(R¹⁰)(R¹¹);
each R¹⁰ is independently selected from hydrogen, C₁₋₆alkyl, C₁₋₆ haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from halogen, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl;
each R¹¹ is independently selected from hydrogen, C₁₋₆alkyl, and C₁₋₆haloalkyl;
each R¹² is independently selected from hydrogen, C₁₋₆alkyl, and C₁₋₆haloalkyl; and
each R¹³ is independently selected C₁₋₆alkyl, C₁₋₆haloalkyl, C₂₋₆alkenyl, C₂₋₆alkynyl, C₃₋₆ cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three groups selected from halogen, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl.

In some embodiments is a compound of Formula (Ia′) or (Ia), or a pharmaceutically acceptable salt or solvate thereof, wherein each R³ is independently selected from H, halogen, C₁₋₆alkyl, C₁₋₆haloalkyl, and —OR¹⁰. In some embodiments is a compound of Formula (Ia′) or (Ia), or a pharmaceutically acceptable salt or solvate thereof, wherein each R³ is independently selected from H, halogen, C₁₋₆alkyl, and C₁₋₆haloalkyl. In some embodiments is a compound of Formula (Ia′) or (Ia), or a pharmaceutically acceptable salt or solvate thereof, wherein each R³ is independently selected from H, halogen, and C₁₋₆haloalkyl.

In some embodiments is a compound of Formula (Ia′) or (Ia), or a pharmaceutically acceptable salt or solvate thereof, wherein R² is selected from H, halogen, C₁₋₆alkyl, C₁₋₆ haloalkyl, and —OR¹⁰. In some embodiments is a compound of Formula (Ia′) or (Ia), or a pharmaceutically acceptable salt or solvate thereof, wherein R² is H. In some embodiments is a compound of Formula (Ia′) or (Ia), or a pharmaceutically acceptable salt or solvate thereof, wherein R² is halogen. In some embodiments is a compound of Formula (Ia′) or (Ia), or a pharmaceutically acceptable salt or solvate thereof, wherein R² is C₁₋₆alkyl. In some embodiments is a compound of Formula (Ia′) or (Ia), or a pharmaceutically acceptable salt or solvate thereof, wherein R² is C₁₋₆haloalkyl. In some embodiments is a compound of Formula (Ia′) or (Ia), or a pharmaceutically acceptable salt or solvate thereof, wherein R² is —OR¹⁰. In some embodiments is a compound of Formula (Ia′) or (Ia), or a pharmaceutically acceptable salt or solvate thereof, wherein R² is —OH. In some embodiments is a compound of Formula (Ia′) or (Ia), or a pharmaceutically acceptable salt or solvate thereof, wherein R² is —OCH₃.

In some embodiments is a compound of Formula (Ia′) or (Ia), or a pharmaceutically acceptable salt or solvate thereof, wherein R⁴ is selected from H, halogen, C₁₋₆alkyl, and C₃₋₆ cycloalkyl. In some embodiments is a compound of Formula (Ia′) or (Ia), or a pharmaceutically acceptable salt or solvate thereof, wherein R⁴ is H. In some embodiments is a compound of Formula (Ia′) or (Ia), or a pharmaceutically acceptable salt or solvate thereof, wherein R⁴ is halogen. In some embodiments is a compound of Formula (Ia′) or (Ia), or a pharmaceutically acceptable salt or solvate thereof, wherein R⁴ is C₁₋₆alkyl. In some embodiments is a compound of Formula (Ia′) or (Ia), or a pharmaceutically acceptable salt or solvate thereof, wherein R⁴ is C₃₋₆cycloalkyl.

In some embodiments is a compound of Formula (Ia′) or (Ia), or a pharmaceutically acceptable salt or solvate thereof, wherein Z¹, Z², and Z³ are CR⁵. In some embodiments is a compound of Formula (Ia′) or (Ia), or a pharmaceutically acceptable salt or solvate thereof, wherein Z¹ is N; and Z² and Z³ are CR⁵. In some embodiments is a compound of Formula (Ia′) or (Ia), or a pharmaceutically acceptable salt or solvate thereof, wherein Z² is N; and Z¹ and Z³ are CR⁵. In some embodiments is a compound of Formula (Ia′) or (Ia), or a pharmaceutically acceptable salt or solvate thereof, wherein Z³ is N; and Z¹ and Z² are CR⁵. In some embodiments is a compound of Formula (Ia′) or (Ia), or a pharmaceutically acceptable salt or solvate thereof, wherein Z¹ is CR⁵; and Z² and Z³ are N. In some embodiments is a compound of Formula (Ia′) or (Ia), or a pharmaceutically acceptable salt or solvate thereof, wherein Z² is CR⁵; and Z¹ and Z³ are N. In some embodiments is a compound of Formula (Ia′) or (Ia), or a pharmaceutically acceptable salt or solvate thereof, wherein Z³ is CR⁵; and Z¹ and Z² are N. In some embodiments is a compound of Formula (Ia′) or (Ia), or a pharmaceutically acceptable salt or solvate thereof, wherein each R⁵ is independently selected from H, halogen, C₁₋₆alkyl, and —OR¹⁰. In some embodiments is a compound of Formula (Ia′) or (Ia), or a pharmaceutically acceptable salt or solvate thereof, wherein each R⁵ is H. In some embodiments is a compound of Formula (Ia′) or (Ia), or a pharmaceutically acceptable salt or solvate thereof, wherein Z¹, Z², and Z³ are C(H). In some embodiments is a compound of Formula (Ia′) or (Ia), or a pharmaceutically acceptable salt or solvate thereof, wherein Z¹ is N; and Z² and Z³ are C(H). In some embodiments is a compound of Formula (Ia′) or (Ia), or a pharmaceutically acceptable salt or solvate thereof, wherein Z² is N; and Z¹ and Z³ are C(H). In some embodiments is a compound of Formula (Ia′) or (Ia), or a pharmaceutically acceptable salt or solvate thereof, wherein Z³ is N; and Z¹ and Z² are C(H). In some embodiments is a compound of Formula (Ia′) or (Ia), or a pharmaceutically acceptable salt or solvate thereof, wherein Z¹ is C(H); and Z² and Z³ are N. In some embodiments is a compound of Formula (Ia′) or (Ia), or a pharmaceutically acceptable salt or solvate thereof, wherein Z² is C(H); and Z¹ and Z³ are N. In some embodiments is a compound of Formula (Ia′) or (Ia), or a pharmaceutically acceptable salt or solvate thereof, wherein Z³ is C(H); and Z¹ and Z² are N.

In some embodiments is a compound of Formula (Ia′) or (Ia), or a pharmaceutically acceptable salt or solvate thereof, wherein L¹ is a bond. In some embodiments is a compound of Formula (Ia′) or (Ia), or a pharmaceutically acceptable salt or solvate thereof, wherein L is —O—. In some embodiments is a compound of Formula (Ia′) or (Ia), or a pharmaceutically acceptable salt or solvate thereof, wherein L¹ is —N(R¹⁰)—. In some embodiments is a compound of Formula (Ia′) or (Ia), or a pharmaceutically acceptable salt or solvate thereof, wherein L¹ is —N(H)—. In some embodiments is a compound of Formula (Ia′) or (Ia), or a pharmaceutically acceptable salt or solvate thereof, wherein L¹ is —N(CH₃)—. In some embodiments is a compound of Formula (Ia′) or (Ia), or a pharmaceutically acceptable salt or solvate thereof, wherein L¹ is —C(R¹⁰)(R¹¹)N(R¹⁰)—. In some embodiments is a compound of Formula (Ia′) or (Ia), or a pharmaceutically acceptable salt or solvate thereof, wherein L¹ is —CH₂N(H)—. In some embodiments is a compound of Formula (Ia′) or (Ia), or a pharmaceutically acceptable salt or solvate thereof, wherein L¹ is —N(R¹⁰)C(R¹⁰)(R¹¹)—. In some embodiments is a compound of Formula (Ia′) or (Ia), or a pharmaceutically acceptable salt or solvate thereof, wherein L¹ is —N(H)CH₂—.

In some embodiments is a compound of Formula (Ia′) or (Ia), or a pharmaceutically acceptable salt or solvate thereof, wherein R¹ is selected from C₃₋₈cycloalkyl and C₂₋₉heterocycloalkyl, wherein C₃₋₈cycloalkyl and C₂₋₉heterocycloalkyl are optionally substituted with one, two, or three R⁶. In some embodiments is a compound of Formula (Ia′) or (Ia), or a pharmaceutically acceptable salt or solvate thereof, wherein R¹ is C₂₋₉heterocycloalkyl optionally substituted with one, two, or three R⁶. In some embodiments is a compound of Formula (Ia′) or (Ia), or a pharmaceutically acceptable salt or solvate thereof, wherein R¹ is C₂₋₉heterocycloalkyl selected from piperidinyl, piperazinyl, morpholinyl, tetrahydropyranyl, tetrahydrofuranyl, pyrrolidinyl, oxetanyl, azetidinyl, aziridinyl, azepanyl, diazepanyl, 6-azaspiro[2.5]octanyl, 4,7-diazaspiro[2.5]octanyl, 7-oxa-4-azaspiro[2.5]octanyl, 5,8-diazaspiro[3.5]nonanyl, 8-oxa-5-azaspiro[3.5]nonanyl, or 2,6-diazaspiro[3.3]heptanyl, wherein piperidinyl, piperazinyl, morpholinyl, tetrahydropyranyl, tetrahydrofuranyl, pyrrolidinyl, oxetanyl, azetidinyl, aziridinyl, azepanyl, diazepanyl, 6-azaspiro[2.5]octanyl, 4,7-diazaspiro[2.5]octanyl, 7-oxa-4-azaspiro[2.5]octanyl, 5,8-diazaspiro[3.5]nonanyl, 8-oxa-5-azaspiro[3.5]nonanyl, or 2,6-diazaspiro[3.3]heptanyl are optionally substituted with one, two, or three R⁶. In some embodiments is a compound of Formula (Ia′) or (Ia), or a pharmaceutically acceptable salt or solvate thereof, wherein R¹ is

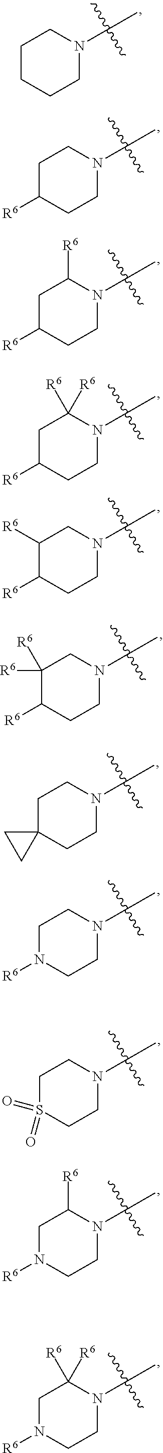

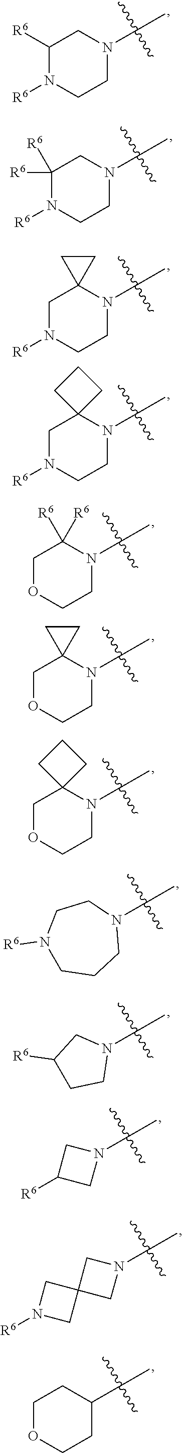

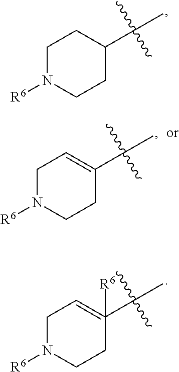

In some embodiments is a compound of Formula (Ia′) or (Ia), or a pharmaceutically acceptable salt or solvate thereof, wherein each R⁶ is independently selected from C₁₋₆alkyl, —OR¹⁰, —C(O)OR¹⁰, —N(R¹²)S(O)₂R¹³, —C(O)R¹³, —C(O)N(R¹⁰)(R¹¹), —S(O)₂R¹³, and —S(O)₂N(R¹⁰)(R¹¹)—. In some embodiments is a compound of Formula (Ia′) or (Ia), or a pharmaceutically acceptable salt or solvate thereof, wherein R¹ is

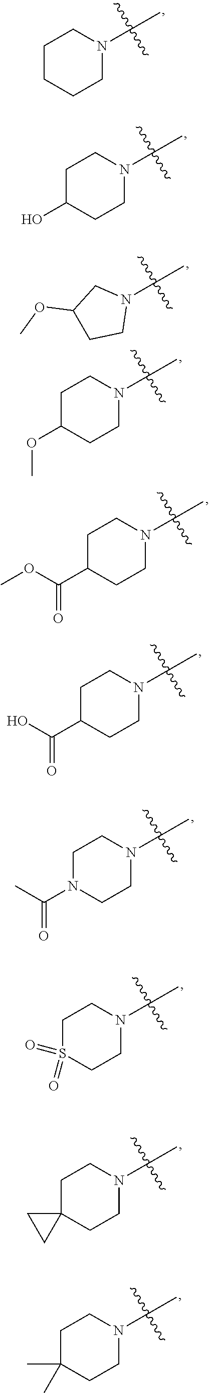

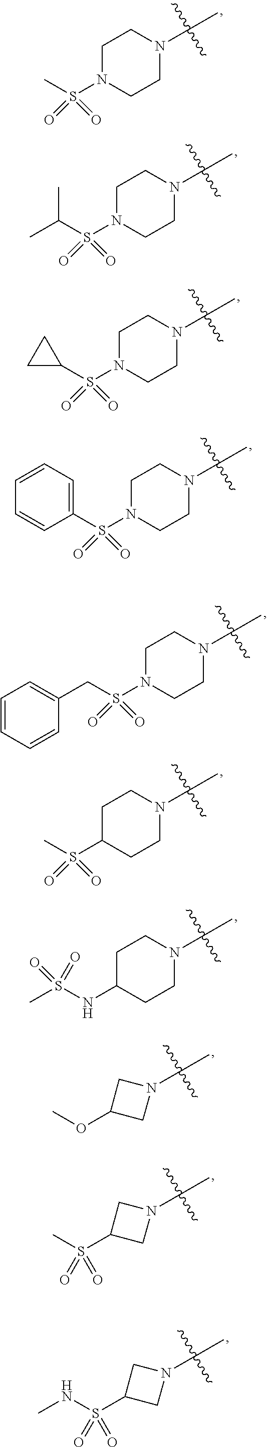

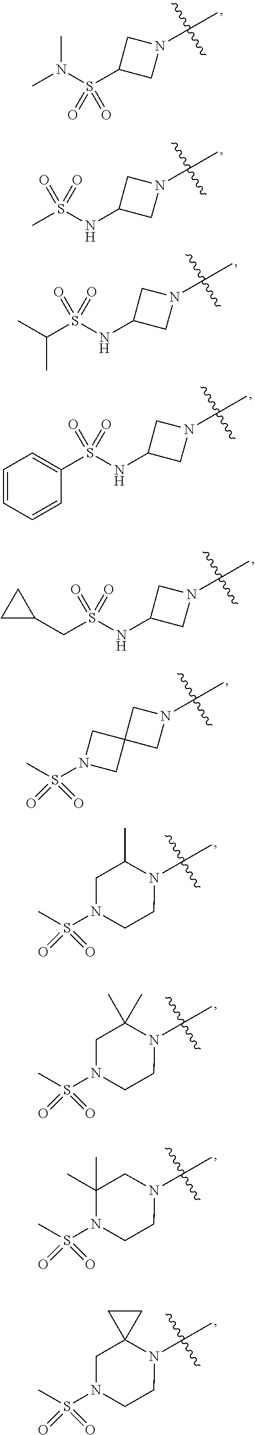

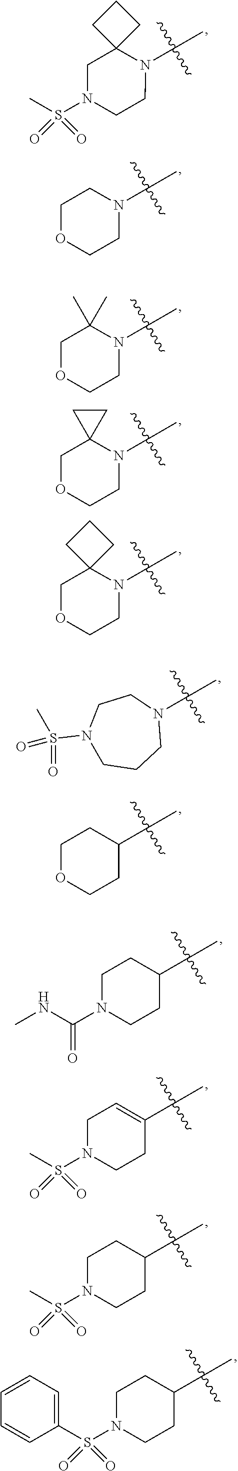

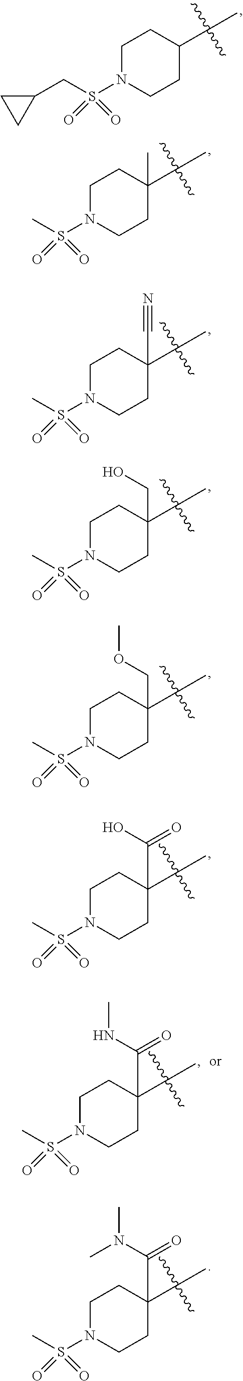

In some embodiments is a compound of Formula (Ia′) or (Ia), or a pharmaceutically acceptable salt or solvate thereof, wherein R¹ is

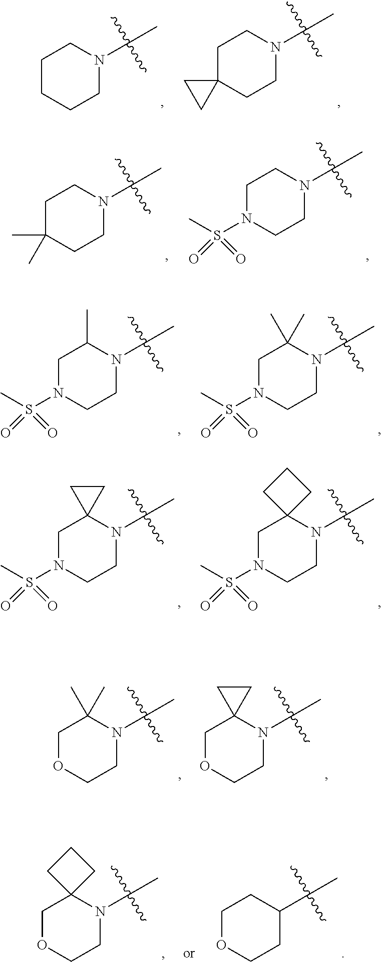

In some embodiments is a compound of Formula (Ia′) or (Ia), or a pharmaceutically acceptable salt or solvate thereof, wherein R¹ is

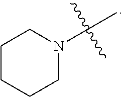 [SMILES: *N1CCCCC1]

In some embodiments is a compound of Formula (Ia′) or (Ia), or a pharmaceutically acceptable salt or solvate thereof, wherein R¹ is

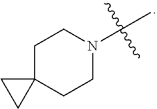 [SMILES: *N1CCC2(CC1)CC2]

In some embodiments is a compound of Formula (Ia′) or (Ia), or a pharmaceutically acceptable salt or solvate thereof, wherein R¹ is

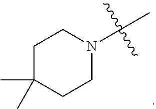 [SMILES: *N1CCC(C)(C)CC1]

In some embodiments is a compound of Formula (Ia′) or (Ia), or a pharmaceutically acceptable salt or solvate thereof, wherein R¹ is

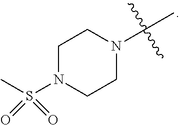 [SMILES: *N1CCN(S(C)(=O)=O)CC1]

In some embodiments is a compound of Formula (Ia′) or (Ia), or a pharmaceutically acceptable salt or solvate thereof, wherein R¹ is

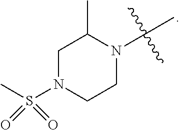 [SMILES: *N1CCN(S(C)(=O)=O)CC1C]

In some embodiments is a compound of Formula (Ia′) or (Ia), or a pharmaceutically acceptable salt or solvate thereof, wherein R¹ is

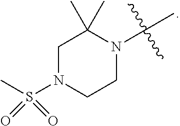 [SMILES: *N1CCN(S(C)(=O)=O)CC1(C)C]

In some embodiments is a compound of Formula (Ia′) or (Ia), or a pharmaceutically acceptable salt or solvate thereof, wherein R¹ is

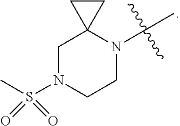 [SMILES: *N1CCN(S(C)(=O)=O)CC12CC2]

In some embodiments is a compound of Formula (Ia′) or (Ia), or a pharmaceutically acceptable salt or solvate thereof, wherein R¹ is

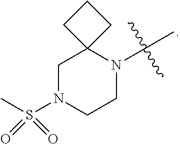 [SMILES: *N1CCN(S(C)(=O)=O)CC12CCC2]

In some embodiments is a compound of Formula (Ia′) or (Ia), or a pharmaceutically acceptable salt or solvate thereof, wherein R¹ is

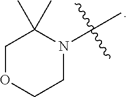 [SMILES: *N1CCOCC1(C)C]

In some embodiments is a compound of Formula (Ia′) or (Ia), or a pharmaceutically acceptable salt or solvate thereof, wherein R¹ is

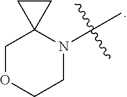 [SMILES: *N1CCOCC12CC2]

In some embodiments is a compound of Formula (Ia′) or (Ia), or a pharmaceutically acceptable salt or solvate thereof, wherein R¹ is

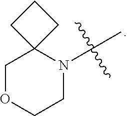 [SMILES: *N1CCOCC12CCC2]

In some embodiments is a compound of Formula (Ia′) or (Ia), or a pharmaceutically acceptable salt or solvate thereof, wherein R¹ is

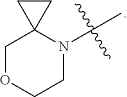 [SMILES: *N1CCOCC12CC2]

In some embodiments is a compound of Formula (Ia′) or (Ia), or a pharmaceutically acceptable salt or solvate thereof, wherein R¹ is

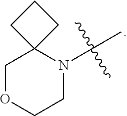 [SMILES: *N1CCOCC12CCC2]

In some embodiments is a compound of Formula (Ia′) or (Ia), or a pharmaceutically acceptable salt or solvate thereof, wherein R¹ is

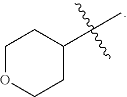 [SMILES: *C1CCOCC1]

In some embodiments is a compound of Formula (Ia′) or (Ia), or a pharmaceutically acceptable salt or solvate thereof, wherein R¹ is C₃₋₈cycloalkyl optionally substituted with one, two, or three R⁶.

In some embodiments is a compound of Formula (Ia′) or (Ia), or a pharmaceutically acceptable salt or solvate thereof, wherein R¹ is selected from C₆₋₁₀aryl and C₁₋₉heteroaryl, wherein C₆₋₁₀aryl and C₁₋₉heteroaryl are substituted with one, two, or three R⁷. In some embodiments is a compound of Formula (Ia′) or (Ia), or a pharmaceutically acceptable salt or solvate thereof, wherein R¹ is C₁₋₉heteroaryl substituted with one, two, or three R⁷. In some embodiments is a compound of Formula (Ia′) or (Ia), or a pharmaceutically acceptable salt or solvate thereof, wherein R¹ is C₁₋₉heteroaryl selected from pyridinyl, pyrimidinyl, pyrazinyl, pyridazinyl, triazinyl, oxazolyl, thiazolyl, pyrazolyl, furanyl, thienyl, pyrrolyl, imidazolyl, triazolyl, tetrazolyl, isoxazolyl, isothiazolyl, oxadiazolyl, and thiadiazolyl, wherein pyridinyl, pyrimidinyl, pyrazinyl, pyridazinyl, triazinyl, oxazolyl, thiazolyl, pyrazolyl, furanyl, thienyl, pyrrolyl, imidazolyl, triazolyl, tetrazolyl, isoxazolyl, isothiazolyl, oxadiazolyl, and thiadiazolyl are substituted with one, two, or three R⁷. In some embodiments is a compound of Formula (Ia′) or (Ia), or a pharmaceutically acceptable salt or solvate thereof, wherein R¹ is

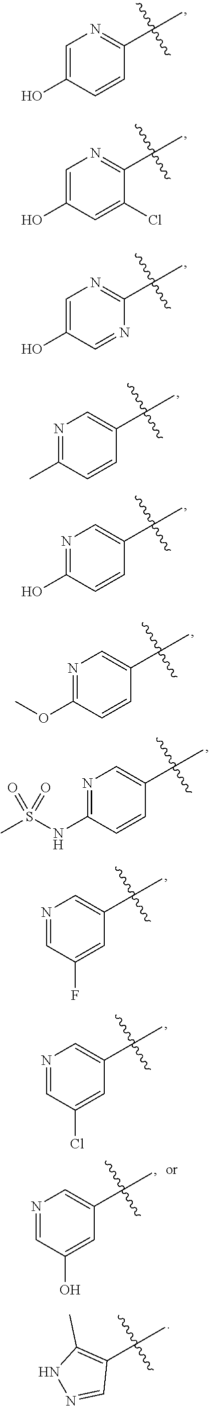

In some embodiments is a compound of Formula (Ia′) or (Ia), or a pharmaceutically acceptable salt or solvate thereof, wherein R¹ is phenyl substituted with one, two, or three R⁷.

In some embodiments is a compound of Formula (II), or a pharmaceutically acceptable salt or solvate thereof:

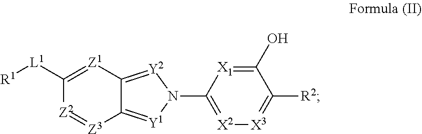

wherein:
X¹, X², and X³ are each independently CR³ or N;
Y¹ and Y² are each independently CR⁴ or N;
Z¹, Z², and Z³ are each independently CR⁵ or N; L¹ is selected from a bond, —O—, —N(R¹⁰)—, —S(O)₂—, —C(R¹⁰)(R¹¹)N(R¹⁰)—, and —N(R¹⁰)C(R¹⁰)(R¹¹)—;
R¹ is selected from:
   a) C₃₋₈cycloalkyl and C₂₋₉heterocycloalkyl, wherein C₃₋₈cycloalkyl and C₂₋₉heterocycloalkyl are optionally substituted with one, two, or three R⁶; or
   b) C₆₋₁₀aryl and C₁₋₉heteroaryl, wherein C₆₋₁₀aryl and C₁₋₉heteroaryl are substituted with one, two, or three R⁷;
R² is selected from H, halogen, —CN, C₁₋₆alkyl, C₁₋₆haloalkyl, C₂₋₆alkenyl, C₂₋₆alkynyl, C₃₋₆ cycloalkyl, C₂₋₉heterocycloalkyl, C₆₋₁₀aryl, C₁₋₉heteroaryl, —OR¹⁰, —SR¹⁰, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R¹³, —S(O)₂N(R¹⁰)(R¹¹)—, S(═O)(═NH)N(R¹⁰)(R¹¹), —CH₂C(O)N(R¹⁰)(R¹¹), —CH₂N(R¹²)C(O)R¹³, —CH₂S(O)₂R¹³, and —CH₂S(O)₂N(R¹⁰)(R¹¹), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three groups selected from halogen, C₁₋₆alkyl, C₁₋₆haloalkyl, —OR¹⁰, and —N(R¹⁰)(R¹¹);
each R³, each R⁴, and each R⁵ are each independently selected from H, halogen, —CN, C₁₋₆ alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹⁰, —SR¹⁰, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R¹³, —S(O)₂N(R¹⁰)(R¹¹)—, S(═O)(═NH)N(R¹⁰)(R¹¹), —CH₂C(O)N(R¹⁰)(R¹¹), —CH₂N(R¹²)C(O)R¹³, —CH₂S(O)₂R¹³, and —CH₂S(O)₂N(R¹⁰)(R¹¹), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three groups selected from halogen, C₁₋₆alkyl, C₁₋₆haloalkyl, —OR¹⁰, and —N(R¹⁰)(R¹¹);
each R⁶ and each R⁷ are each independently selected from halogen, oxo, —CN, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹⁰, —SR¹⁰, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R¹³, —S(O)₂N(R¹⁰)(R¹¹)—, S(═O)(═NH)N(R¹⁰)(R¹¹), —CH₂C(O)N(R¹⁰)(R¹¹), —CH₂N(R¹²)C(O)R¹³, —CH₂S(O)₂R¹³, and —CH₂S(O)₂N(R¹⁰)(R¹¹), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three groups selected from halogen, C₁₋₆alkyl, C₁₋₆haloalkyl, —OR¹⁰, and —N(R¹⁰)(R¹¹);
each R¹⁰ is independently selected from hydrogen, C₁₋₆alkyl, C₁₋₆ haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from halogen, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl;
each R¹¹ is independently selected from hydrogen, C₁₋₆alkyl, and C₁₋₆haloalkyl;
each R¹² is independently selected from hydrogen, C₁₋₆alkyl, and C₁₋₆haloalkyl; and
each R¹³ is independently selected C₁₋₆alkyl, C₁₋₆haloalkyl, C₂₋₆alkenyl, C₂₋₆alkynyl, C₃₋₆ cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three groups selected from halogen, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl.

In some embodiments is a compound of Formula (II), or a pharmaceutically acceptable salt or solvate thereof, wherein X¹, X², and X³ are each CR³. In some embodiments is a compound of Formula (II), or a pharmaceutically acceptable salt or solvate thereof, wherein X¹, X², and X³ are each CR³ and each R³ is independently selected from H, halogen, C₁₋₆alkyl, C₁₋₆haloalkyl, and —OR¹⁰. In some embodiments is a compound of Formula (II), or a pharmaceutically acceptable salt or solvate thereof, wherein X¹, X², and X³ are each CR³ and each R³ is independently selected from H, halogen, C₁₋₆alkyl, and C₁₋₆ haloalkyl. In some embodiments is a compound of Formula (II), or a pharmaceutically acceptable salt or solvate thereof, wherein X¹, X², and X³ are each CR³ and each R³ is independently selected from H, halogen, and C₁₋₆haloalkyl. In some embodiments is a compound of Formula (II), or a pharmaceutically acceptable salt or solvate thereof, wherein X¹ is C(H), X² is C(H), and X³ is C(CF₃). In some embodiments is a compound of Formula (II), or a pharmaceutically acceptable salt or solvate thereof, wherein X¹ is C(F), X² is C(H), and X³ is C(CF₃). In some embodiments is a compound of Formula (II), or a pharmaceutically acceptable salt or solvate thereof, wherein X¹ is C(Cl), X² is C(H), and X³ is C(CF₃). In some embodiments is a compound of Formula (II), or a pharmaceutically acceptable salt or solvate thereof, wherein X¹ is C(H), X² is C(H), and X³ is C(F). In some embodiments is a compound of Formula (II), or a pharmaceutically acceptable salt or solvate thereof, wherein X¹ is C(H), X² is C(H), and X³ is C(Cl).

In some embodiments is a compound of Formula (II), or a pharmaceutically acceptable salt or solvate thereof, wherein R² is selected from H, halogen, C₁₋₆alkyl, C₁₋₆ haloalkyl, and —OR¹⁰. In some embodiments is a compound of Formula (II), or a pharmaceutically acceptable salt or solvate thereof, wherein R² is H. In some embodiments is a compound of Formula (II), or a pharmaceutically acceptable salt or solvate thereof, wherein R² is halogen. In some embodiments is a compound of Formula (II), or a pharmaceutically acceptable salt or solvate thereof, wherein R² is C₁₋₆alkyl. In some embodiments is a compound of Formula (II), or a pharmaceutically acceptable salt or solvate thereof, wherein R² is C₁₋₆haloalkyl. In some embodiments is a compound of Formula (II), or a pharmaceutically acceptable salt or solvate thereof, wherein R² is —OR¹⁰. In some embodiments is a compound of Formula (II), or a pharmaceutically acceptable salt or solvate thereof, wherein R² is —OH. In some embodiments is a compound of Formula (II), or a pharmaceutically acceptable salt or solvate thereof, wherein R² is —OCH₃.

In some embodiments is a compound of Formula (II), or a pharmaceutically acceptable salt or solvate thereof, wherein Y¹ is N and Y² is CR⁴. In some embodiments is a compound of Formula (II), or a pharmaceutically acceptable salt or solvate thereof, wherein Y¹ is CR⁴ and Y² is CR⁴. In some embodiments is a compound of Formula (II), or a pharmaceutically acceptable salt or solvate thereof, wherein Y¹ is CR⁴ and Y² is N. In some embodiments is a compound of Formula (II), or a pharmaceutically acceptable salt or solvate thereof, wherein each R⁴ is independently selected from H, halogen, C₁₋₆alkyl, and C₃₋₆ cycloalkyl. In some embodiments is a compound of Formula (II), or a pharmaceutically acceptable salt or solvate thereof, wherein Y¹ is N and Y² is C(H). In some embodiments is a compound of Formula (II), or a pharmaceutically acceptable salt or solvate thereof, wherein Y¹ is C(H) and Y² is C(H). In some embodiments is a compound of Formula (II), or a pharmaceutically acceptable salt or solvate thereof, wherein Y¹ is C(H) and Y² is N. In some embodiments is a compound of Formula (II), or a pharmaceutically acceptable salt or solvate thereof, wherein Y¹ is N and Y² is N.

In some embodiments is a compound of Formula (II), or a pharmaceutically acceptable salt or solvate thereof, wherein Z¹, Z², and Z³ are CR⁵. In some embodiments is a compound of Formula (II), or a pharmaceutically acceptable salt or solvate thereof, wherein Z¹ is N; and Z² and Z³ are CR⁵. In some embodiments is a compound of Formula (II), or a pharmaceutically acceptable salt or solvate thereof, wherein Z² is N; and Z¹ and Z³ are CR⁵. In some embodiments is a compound of Formula (II), or a pharmaceutically acceptable salt or solvate thereof, wherein Z³ is N; and Z¹ and Z² are CR⁵. In some embodiments is a compound of Formula (II), or a pharmaceutically acceptable salt or solvate thereof, wherein Z¹ is CR⁵; and Z² and Z³ are N. In some embodiments is a compound of Formula (II), or a pharmaceutically acceptable salt or solvate thereof, wherein Z² is CR⁵; and Z¹ and Z³ are N. In some embodiments is a compound of Formula (II), or a pharmaceutically acceptable salt or solvate thereof, wherein Z³ is CR⁵; and Z¹ and Z² are N. In some embodiments is a compound of Formula (II), or a pharmaceutically acceptable salt or solvate thereof, wherein each R⁵ is independently selected from H, halogen, C₁₋₆alkyl, and —OR¹⁰. In some embodiments is a compound of Formula (II), or a pharmaceutically acceptable salt or solvate thereof, wherein each R⁵ is H. In some embodiments is a compound of Formula (II), or a pharmaceutically acceptable salt or solvate thereof, wherein Z¹, Z², and Z³ are C(H). In some embodiments is a compound of Formula (II), or a pharmaceutically acceptable salt or solvate thereof, wherein Z¹ is N; and Z² and Z³ are C(H). In some embodiments is a compound of Formula (II), or a pharmaceutically acceptable salt or solvate thereof, wherein Z² is N; and Z¹ and Z³ are C(H). In some embodiments is a compound of Formula (II), or a pharmaceutically acceptable salt or solvate thereof, wherein Z³ is N; and Z¹ and Z² are C(H). In some embodiments is a compound of Formula (II), or a pharmaceutically acceptable salt or solvate thereof, wherein Z¹ is C(H); and Z² and Z³ are N. In some embodiments is a compound of Formula (II), or a pharmaceutically acceptable salt or solvate thereof, wherein Z² is C(H); and Z¹ and Z³ are N. In some embodiments is a compound of Formula (II), or a pharmaceutically acceptable salt or solvate thereof, wherein Z³ is C(H); and Z¹ and Z² are N.

In some embodiments is a compound of Formula (II), or a pharmaceutically acceptable salt or solvate thereof, wherein L¹ is a bond. In some embodiments is a compound of Formula (II), or a pharmaceutically acceptable salt or solvate thereof, wherein L¹ is —O—. In some embodiments is a compound of Formula (II), or a pharmaceutically acceptable salt or solvate thereof, wherein L¹ is —N(R¹⁰)—. In some embodiments is a compound of Formula (II), or a pharmaceutically acceptable salt or solvate thereof, wherein L¹ is —N(H)—. In some embodiments is a compound of Formula (II), or a pharmaceutically acceptable salt or solvate thereof, wherein L¹ is —N(CH₃)—. In some embodiments is a compound of Formula (II), or a pharmaceutically acceptable salt or solvate thereof, wherein L¹ is —C(R¹⁰)(R¹¹)N(R¹⁰)—. In some embodiments is a compound of Formula (II), or a pharmaceutically acceptable salt or solvate thereof, wherein L¹ is —CH₂N(H)—. In some embodiments is a compound of Formula (II), or a pharmaceutically acceptable salt or solvate thereof, wherein L¹ is —N(R¹⁰)C(R¹⁰)(R¹¹)—. In some embodiments is a compound of Formula (II), or a pharmaceutically acceptable salt or solvate thereof, wherein L¹ is —N(H)CH₂—.

In some embodiments is a compound of Formula (II), or a pharmaceutically acceptable salt or solvate thereof, wherein R¹ is selected from C₃₋₈cycloalkyl and C₂₋₉heterocycloalkyl, wherein C₃₋₈cycloalkyl and C₂₋₉heterocycloalkyl are optionally substituted with one, two, or three R⁶. In some embodiments is a compound of Formula (II), or a pharmaceutically acceptable salt or solvate thereof, wherein R¹ is C₂₋₉heterocycloalkyl optionally substituted with one, two, or three R⁶. In some embodiments is a compound of Formula (II), or a pharmaceutically acceptable salt or solvate thereof, wherein R¹ is C₂₋₉heterocycloalkyl selected from piperidinyl, piperazinyl, morpholinyl, tetrahydropyranyl, tetrahydrofuranyl, pyrrolidinyl, oxetanyl, azetidinyl, aziridinyl, azepanyl, diazepanyl, 6-azaspiro[2.5]octanyl, 4,7-diazaspiro[2.5]octanyl, 7-oxa-4-azaspiro[2.5]octanyl, 5,8-diazaspiro[3.5]nonanyl, 8-oxa-5-azaspiro[3.5]nonanyl, or 2,6-diazaspiro[3.3]heptanyl, wherein piperidinyl, piperazinyl, morpholinyl, tetrahydropyranyl, tetrahydrofuranyl, pyrrolidinyl, oxetanyl, azetidinyl, aziridinyl, azepanyl, diazepanyl, 6-azaspiro[2.5]octanyl, 4,7-diazaspiro[2.5]octanyl, 7-oxa-4-azaspiro[2.5]octanyl, 5,8-diazaspiro[3.5]nonanyl, 8-oxa-5-azaspiro[3.5]nonanyl, or 2,6-diazaspiro[3.3]heptanyl are optionally substituted with one, two, or three R⁶. In some embodiments is a compound of Formula (II), or a pharmaceutically acceptable salt or solvate thereof, wherein R¹ is

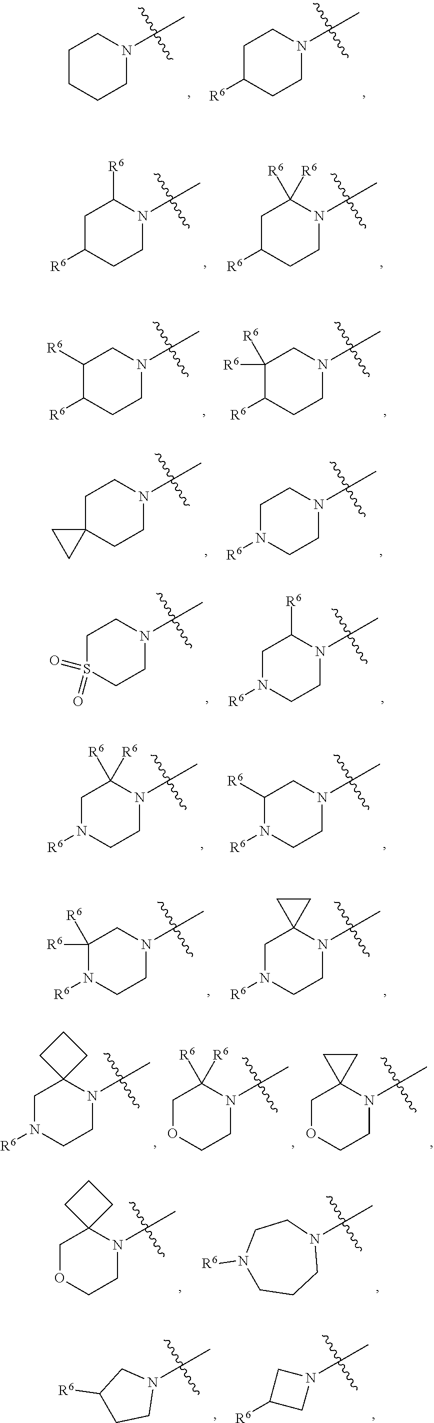

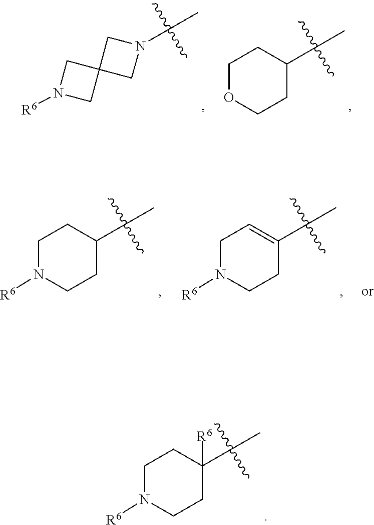

In some embodiments is a compound of Formula (II), or a pharmaceutically acceptable salt or solvate thereof, wherein each R⁶ is independently selected from C₁₋₆alkyl, —OR¹⁰, —C(O)OR¹⁰, —N(R¹²)S(O)₂R¹³, —C(O)R¹³, —C(O)N(R¹⁰)(R¹¹), —S(O)₂R¹³, and —S(O)₂N(R¹⁰)(R¹¹)—. In some embodiments is a compound of Formula (II), or a pharmaceutically acceptable salt or solvate thereof, wherein R¹ is

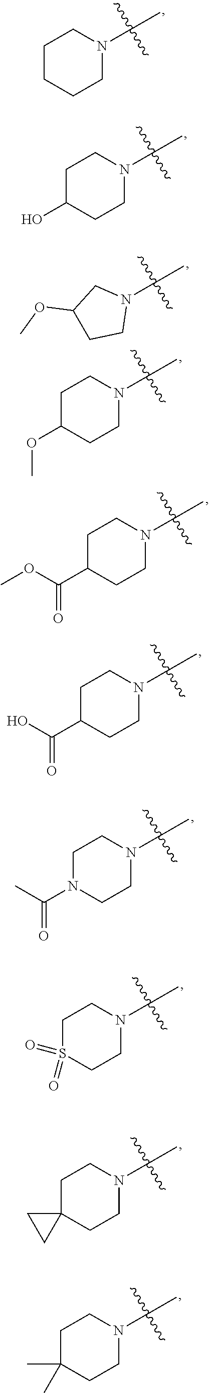

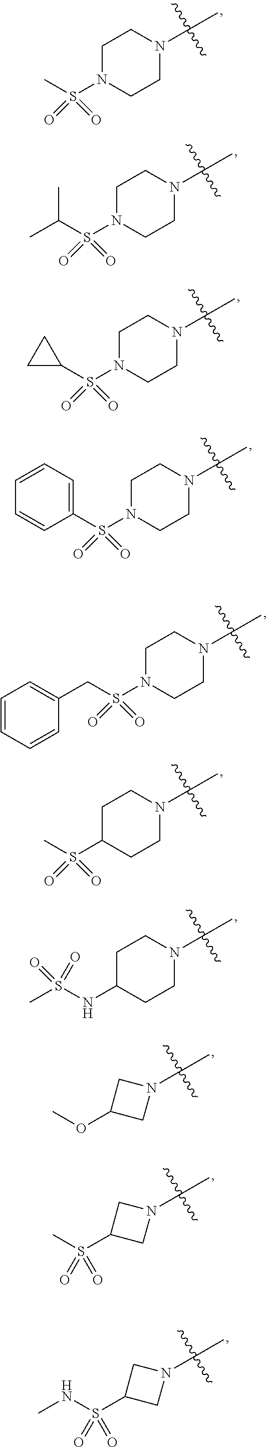

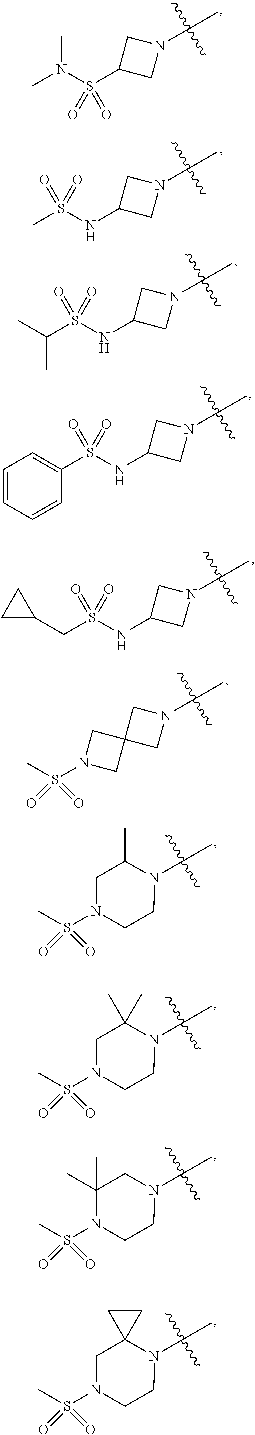

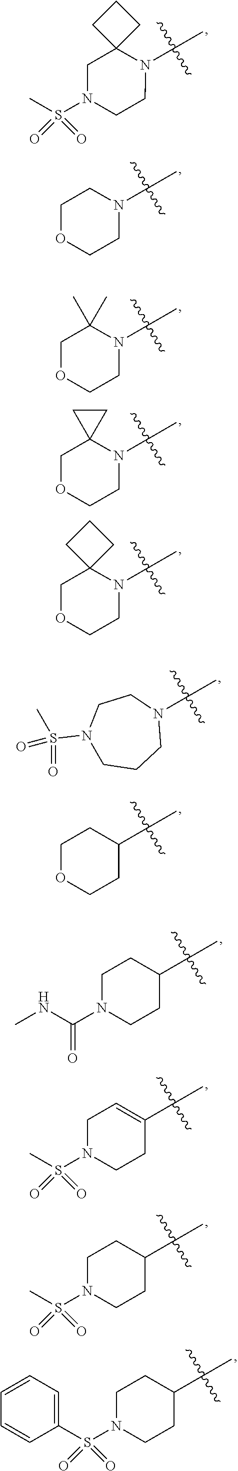

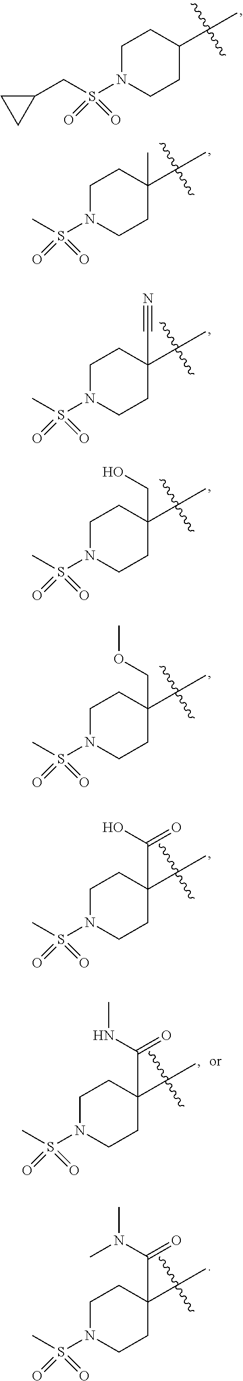

In some embodiments is a compound of Formula (II), or a pharmaceutically acceptable salt or solvate thereof, wherein R¹ is

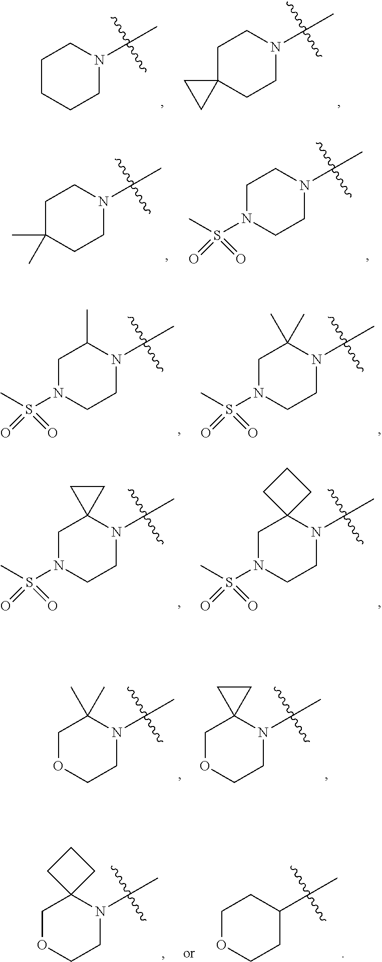

In some embodiments is a compound of Formula (II), or a pharmaceutically acceptable salt or solvate thereof, wherein R¹ is

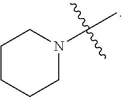 [SMILES: *N1CCCCC1]

In some embodiments is a compound of Formula (II), or a pharmaceutically acceptable salt or solvate thereof, wherein R¹ is

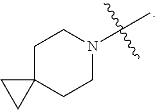 [SMILES: *N1CCC2(CC1)CC2]

In some embodiments is a compound of Formula (II), or a pharmaceutically acceptable salt or solvate thereof, wherein R¹ is

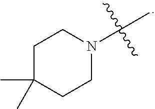 [SMILES: *N1CCC(C)(C)CC1]

In some embodiments is a compound of Formula (II), or a pharmaceutically acceptable salt or solvate thereof, wherein R¹ is

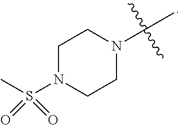 [SMILES: *N1CCN(S(C)(=O)=O)CC1]

In some embodiments is a compound of Formula (II), or a pharmaceutically acceptable salt or solvate thereof, wherein R¹ is

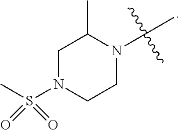 [SMILES: *N1CCN(S(C)(=O)=O)CC1C]

In some embodiments is a compound of Formula (II), or a pharmaceutically acceptable salt or solvate thereof, wherein R¹ is

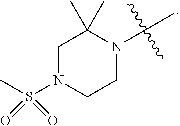 [SMILES: *N1CCN(S(C)(=O)=O)CC1(C)C]

In some embodiments is a compound of Formula (II), or a pharmaceutically acceptable salt or solvate thereof, wherein R¹ is

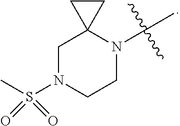 [SMILES: *N1CCN(S(C)(=O)=O)CC12CC2]

In some embodiments is a compound of Formula (II), or a pharmaceutically acceptable salt or solvate thereof, wherein R¹ is

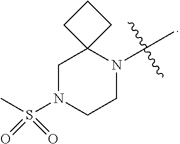 [SMILES: *N1CCN(S(C)(=O)=O)CC12CCC2]

In some embodiments is a compound of Formula (II), or a pharmaceutically acceptable salt or solvate thereof, wherein R¹ is

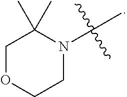 [SMILES: *N1CCOCC1(C)C]

In some embodiments is a compound of Formula (Ia), or a pharmaceutically acceptable salt or solvate thereof, wherein R¹ is

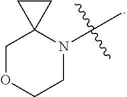 [SMILES: *N1CCOCC12CC2]

In some embodiments is a compound of Formula (Ia), or a pharmaceutically acceptable salt or solvate thereof, wherein R¹ is

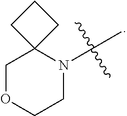 [SMILES: *N1CCOCC12CCC2]

In some embodiments is a compound of Formula (II), or a pharmaceutically acceptable salt or solvate thereof, wherein R¹ is

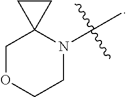 [SMILES: *N1CCOCC12CC2]

In some embodiments is a compound of Formula (II), or a pharmaceutically acceptable salt or solvate thereof, wherein R¹ is

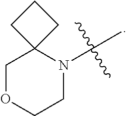 [SMILES: *N1CCOCC12CCC2]

In some embodiments is a compound of Formula (II), or a pharmaceutically acceptable salt or solvate thereof, wherein R¹ is

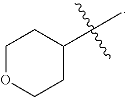 [SMILES: *C1CCOCC1]

In some embodiments is a compound of Formula (II), or a pharmaceutically acceptable salt or solvate thereof, wherein R¹ is C₃₋₈cycloalkyl optionally substituted with one, two, or three R⁶.

In some embodiments is a compound of Formula (II), or a pharmaceutically acceptable salt or solvate thereof, wherein R¹ is selected from C₆₋₁₀aryl and C₁₋₉heteroaryl, wherein C₆₋₁₀aryl and C₁₋₉heteroaryl are substituted with one, two, or three R⁷. In some embodiments is a compound of Formula (II), or a pharmaceutically acceptable salt or solvate thereof, wherein R¹ is C₁₋₉heteroaryl substituted with one, two, or three R⁷. In some embodiments is a compound of Formula (II), or a pharmaceutically acceptable salt or solvate thereof, wherein R¹ is C₁₋₉heteroaryl selected from pyridinyl, pyrimidinyl, pyrazinyl, pyridazinyl, triazinyl, oxazolyl, thiazolyl, pyrazolyl, furanyl, thienyl, pyrrolyl, imidazolyl, triazolyl, tetrazolyl, isoxazolyl, isothiazolyl, oxadiazolyl, and thiadiazolyl, wherein pyridinyl, pyrimidinyl, pyrazinyl, pyridazinyl, triazinyl, oxazolyl, thiazolyl, pyrazolyl, furanyl, thienyl, pyrrolyl, imidazolyl, triazolyl, tetrazolyl, isoxazolyl, isothiazolyl, oxadiazolyl, and thiadiazolyl are substituted with one, two, or three R⁷. In some embodiments is a compound of Formula (II), or a pharmaceutically acceptable salt or solvate thereof, wherein R¹ is

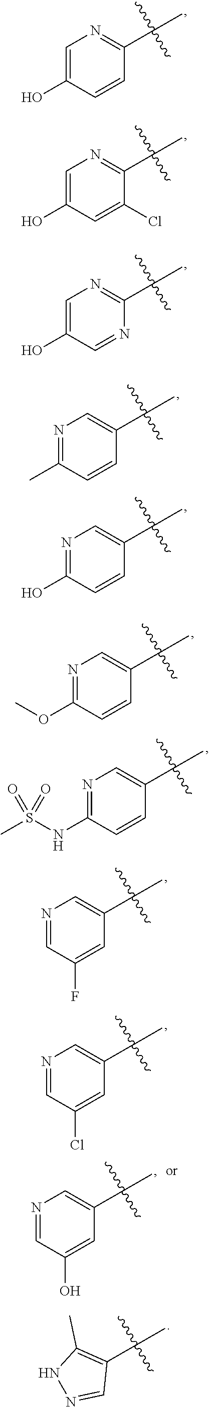

In some embodiments is a compound of Formula (II), or a pharmaceutically acceptable salt or solvate thereof, wherein R¹ is phenyl substituted with one, two, or three R⁷.

Any combination of the groups described above for the various variables is contemplated herein. Throughout the specification, groups and substituents thereof are chosen by one skilled in the field to provide stable moieties and compounds.

In some embodiments, compounds described herein include, but are not limited to, those described in Table 1.

TABLE 1

| Compound | Structure | Chemical Name |
|---|---|---|
| 1 | 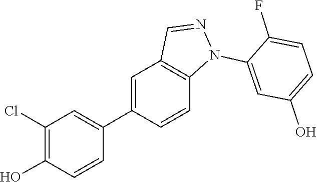 [SMILES: Oc1ccc(F)c(-n2ncc3cc(-c4ccc(O)c(Cl)c4)ccc32)c1] | 2-Chloro-4-(1-(2-fluoro-5-hydroxyphenyl)-1H- indazol-5-yl)phenol |
| 1.01 | 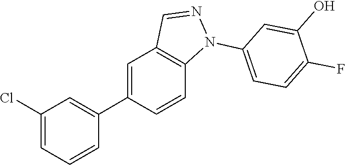 [SMILES: Oc1cc(-n2ncc3cc(-c4cccc(Cl)c4)ccc32)ccc1F] | 5-(5-(3-Chlorophenyl)-1H-indazol-1-yl)-2- fluorophenol |
| 1.02 | 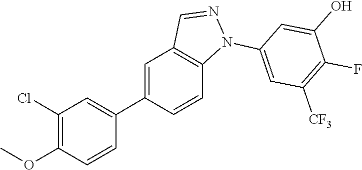 [SMILES: COc1ccc(-c2ccc3c(cnn3-c3cc(O)c(F)c(C(F)(F)F)c3)c2)cc1Cl] | 5-(5-(3-Chloro-4-methoxyphenyl)-1H-indazol- 1-yl)-2-fluoro-3-(trifluoromethyl)phenol |
| 1.03 | 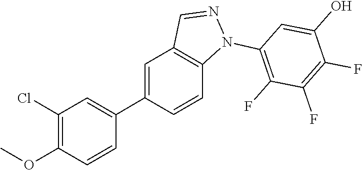 [SMILES: COc1ccc(-c2ccc3c(cnn3-c3cc(O)c(F)c(F)c3F)c2)cc1Cl] | 5-(5-(3-Chloro-4-methoxyphenyl)-1H-indazol- 1-yl)-2,3,4-trifluorophenol |
| 1.04 | 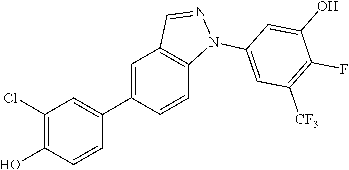 [SMILES: Oc1ccc(-c2ccc3c(cnn3-c3cc(O)c(F)c(C(F)(F)F)c3)c2)cc1Cl] | 5-(5-(3-Chloro-4-hydroxyphenyl)-1H-indazol- 1-yl)-2-fluoro-3-(trifluoromethyl)phenol |
| 1.05 | 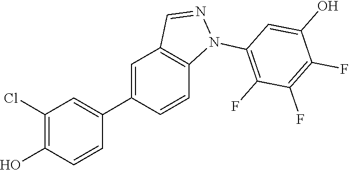 [SMILES: Oc1ccc(-c2ccc3c(cnn3-c3cc(O)c(F)c(F)c3F)c2)cc1Cl] | 5-(5-(3-Chloro-4-hydroxyphenyl)-1H-indazol- 1-yl)-2-fluoro-3-(trifluoromethyl)phenol |
| 1.06 | 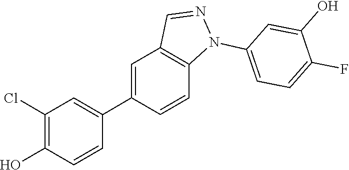 [SMILES: Oc1cc(-n2ncc3cc(-c4ccc(O)c(Cl)c4)ccc32)ccc1F] | 2-Chloro-4-(1-(4-fluoro-3-hydroxyphenyl)-1H- indol-5-yl)phenol |
| 2 | 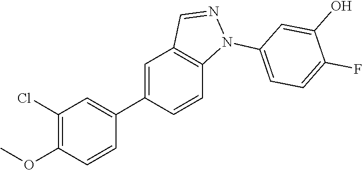 [SMILES: COc1ccc(-c2ccc3c(cnn3-c3ccc(F)c(O)c3)c2)cc1Cl] | 5-(5-(3-Chloro-4-methoxyphenyl)-1H-indazol- 1-yl)-2-fluorophenol |
| 2.01 | 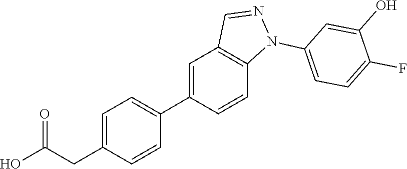 [SMILES: O=C(O)Cc1ccc(-c2ccc3c(cnn3-c3ccc(F)c(O)c3)c2)cc1] | 2-(4-(1-(4-Fluoro-3-hydroxyphenyl)-1H- indazol-5-yl)phenyl)acetic acid |
| 3 | 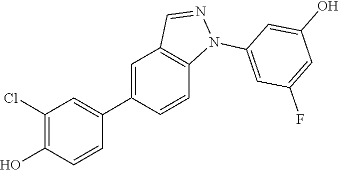 [SMILES: Oc1cc(F)cc(-n2ncc3cc(-c4ccc(O)c(Cl)c4)ccc32)c1] | 2-Chloro-4-(1-(3-fluoro-5-hydroxyphenyl)-1H- indazol-5-yl)phenol |
| 3.01 | 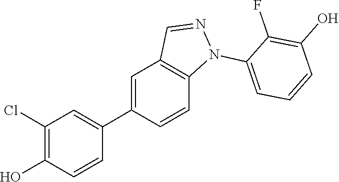 [SMILES: Oc1ccc(-c2ccc3c(cnn3-c3cccc(O)c3F)c2)cc1Cl] | 3-(5-(3-Chloro-4-hydroxyphenyl)-1H-indazol- 1-yl)-2-fluorophenol |
| 3.02 | 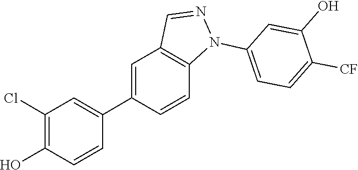 | 2-Chloro-4-(1-(3-hydroxy-4- (trifluoromethyl)phenyl)-1H-indazol-5- yl)phenol |
| 3.03 | 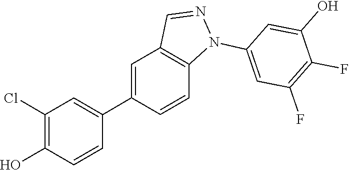 [SMILES: Oc1ccc(-c2ccc3c(cnn3-c3cc(O)c(F)c(F)c3)c2)cc1Cl] | 5-(5-(3-Chloro-4-hydroxyphenyl)-1H-indazol- 1-yl)-2,3-difluorophenol |
| 3.04 | 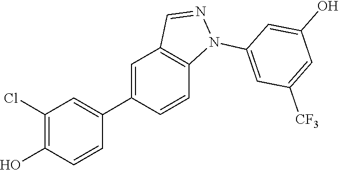 [SMILES: Oc1cc(-n2ncc3cc(-c4ccc(O)c(Cl)c4)ccc32)cc(C(F)(F)F)c1] | 2-Chloro-4-(1-(3-hydroxy-5- (trifluoromethyl)phenyl)-1H-indazol-5- yl)phenol |
| 3.05 | 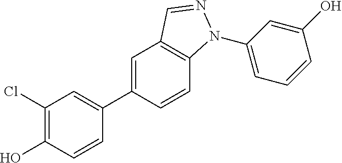 [SMILES: Oc1cccc(-n2ncc3cc(-c4ccc(O)c(Cl)c4)ccc32)c1] | 2-Chloro-4-(1-(3-hydroxyphenyl)-1H-indazol- 5-yl)phenol |
| 4 | 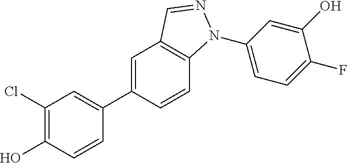 [SMILES: Oc1cc(-n2ncc3cc(-c4ccc(O)c(Cl)c4)ccc32)ccc1F] | 2-Chloro-4-(1-(4-fluoro-3-hydroxyphenyl)-1H- indazol-5-yl)phenol |
| 4.01 | 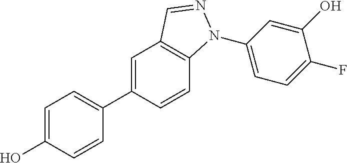 [SMILES: Oc1ccc(-c2ccc3c(cnn3-c3ccc(F)c(O)c3)c2)cc1] | 2-Fluoro-5-(5-(4-hydroxyphenyl)-1H-indazol-1- yl)phenol |
| 4.02 | 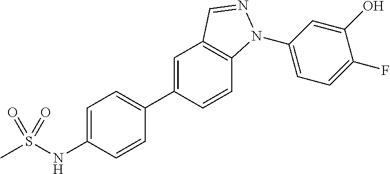 [SMILES: CS(=O)(=O)Nc1ccc(-c2ccc3c(cnn3-c3ccc(F)c(O)c3)c2)cc1] | N-(4-(1-(4-Fluoro-3-hydroxyphenyl)-1H- indazol-5-yl)phenyl)methanesulfonamide |
| 4.03 | 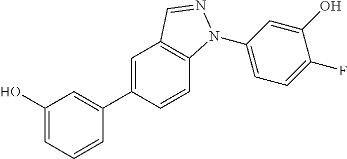 [SMILES: Oc1cccc(-c2ccc3c(cnn3-c3ccc(F)c(O)c3)c2)c1] | 2-Fluoro-5-(5-(3-hydroxyphenyl)-1H-indazol-1- yl)phenol |
| 4.04 | 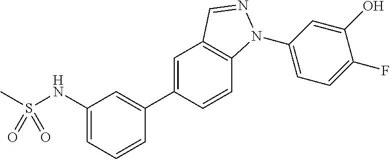 [SMILES: CS(=O)(=O)Nc1cccc(-c2ccc3c(cnn3-c3ccc(F)c(O)c3)c2)c1] | N-(3-(1-(4-Fluoro-3-hydroxyphenyl)-1H- indazol-5-yl)phenyl)methanesulfonamide |
| 4.05 | 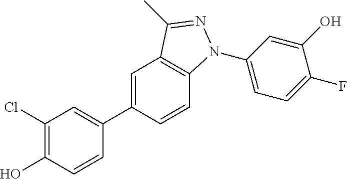 [SMILES: Cc1nn(-c2ccc(F)c(O)c2)c2ccc(-c3ccc(O)c(Cl)c3)cc12] | 2-Chloro-4-(1-(4-fluoro-3-hydroxyphenyl)-3- methyl-1H-indazol-5-yl)phenol |
| 4.06 | 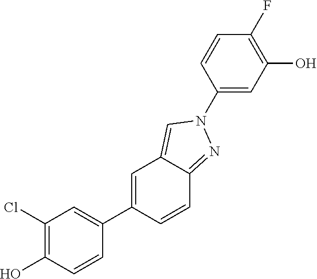 [SMILES: Oc1cc(-n2cc3cc(-c4ccc(O)c(Cl)c4)ccc3n2)ccc1F] | 2-Chloro-4-(2-(4-fluoro-3-hydroxyphenyl)-2H- indazol-5-yl)phenol |
| 4.07 | 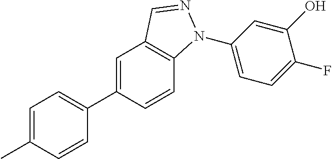 [SMILES: Cc1ccc(-c2ccc3c(cnn3-c3ccc(F)c(O)c3)c2)cc1] | 2-Fluoro-5-(5-(p-tolyl)-1H-indazol-1-yl)phenol |
| 4.08 | 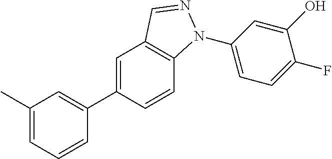 [SMILES: Cc1cccc(-c2ccc3c(cnn3-c3ccc(F)c(O)c3)c2)c1] | 2-Fluoro-5-(5-(m-tolyl)-1H-indazol-1-yl)phenol |
| 4.09 | 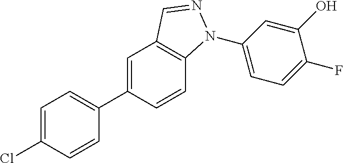 [SMILES: Oc1cc(-n2ncc3cc(-c4ccc(Cl)cc4)ccc32)ccc1F] | 5-(5-(4-Chlorophenyl)-1H-indazol-1-yl)-2- fluorophenol |
| 4.10 | 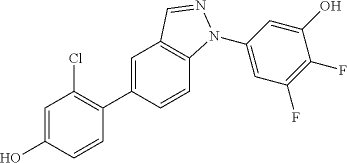 [SMILES: Oc1ccc(-c2ccc3c(cnn3-c3cc(O)c(F)c(F)c3)c2)c(Cl)c1] | 5-(5-(2-Chloro-4-hydroxyphenyl)-1H-indazol- 1-yl)-2,3-difluorophenol |
| 4.11 | 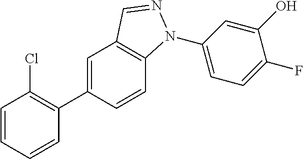 [SMILES: Oc1cc(-n2ncc3cc(-c4ccccc4Cl)ccc32)ccc1F] | 5-(5-(2-Chlorophenyl)-1H-indazol-1-yl)-2- fluorophenol |
| 4.12 | 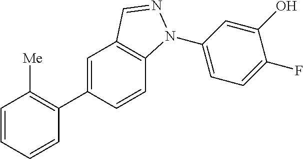 [SMILES: Cc1ccccc1-c1ccc2c(cnn2-c2ccc(F)c(O)c2)c1] | 2-Fluoro-5-(5-(o-tolyl)-1H-indazol-1-yl)phenol |
| 4.13 | 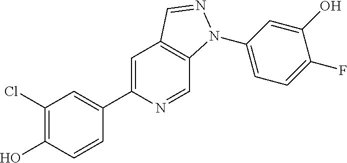 [SMILES: Oc1cc(-n2ncc3cc(-c4ccc(O)c(Cl)c4)ncc32)ccc1F] | 2-Chloro-4-(1-(4-fluoro-3-hydroxyphenyl)-1H- pyrazolo[3,4-c]pyridine-5-yl)phenol |
| 4.14 | 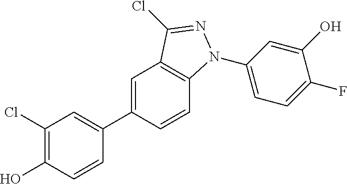 [SMILES: Oc1cc(-n2nc(Cl)c3cc(-c4ccc(O)c(Cl)c4)ccc32)ccc1F] | 2-Chloro-4-(3-chloro-1-(4-fluoro-3- hydroxyphenyl)-1H-indazol-5-yl)phenol |
| 4.15 | 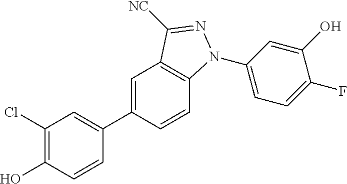 [SMILES: N#Cc1nn(-c2ccc(F)c(O)c2)c2ccc(-c3ccc(O)c(Cl)c3)cc12] | 5-(3-Chloro-4-hydroxyphenyl)-1-(4-fluoro-3- hydroxyphenyl)-1H-indazole-3-carbonitrile |
| 4.16 | 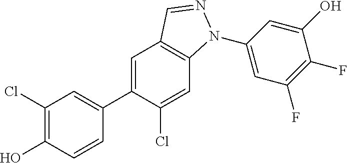 [SMILES: Oc1ccc(-c2cc3cnn(-c4cc(O)c(F)c(F)c4)c3cc2Cl)cc1Cl] | 5-(6-Chloro-5-(3-chloro-4-hydroxyphenyl)-1H- indazol-1-yl)-2,3-difluorophenol |
| 4.17 | 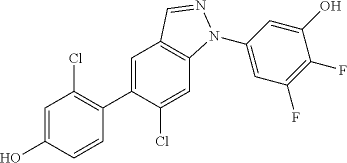 [SMILES: Oc1ccc(-c2cc3cnn(-c4cc(O)c(F)c(F)c4)c3cc2Cl)c(Cl)c1] | 5-(6-Cloro-5-(2-chloro-4-hydroxyphenyl)-1H- indazol-1-yl)-2,3-difluorophenol |
| 5 | 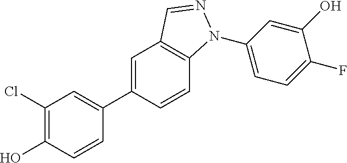 [SMILES: Oc1cc(-n2ncc3cc(-c4ccc(O)c(Cl)c4)ccc32)ccc1F] | 2-Chloro-4-(1-(4-fluoro-3-methoxyphenyl)-1H- indazol-5-yl)phenol |
| 5.01 | 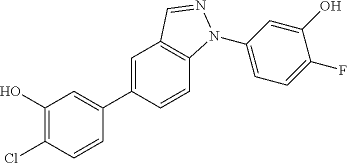 [SMILES: Oc1cc(-n2ncc3cc(-c4ccc(Cl)c(O)c4)ccc32)ccc1F] | 2-Chloro-5-(1-(4-fluoro-3-hydroxyphenyl)-1H- indazol-5-yl)phenol |
| 5.02 | 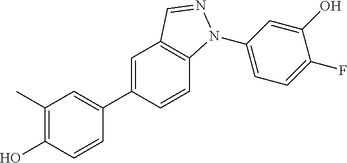 [SMILES: Cc1cc(-c2ccc3c(cnn3-c3ccc(F)c(O)c3)c2)ccc1O] | 4-(1-(4-Fluoro-3-hydroxyphenyl)-1H-indazol-5- yl)-2-methylphenol |
| 5.03 | 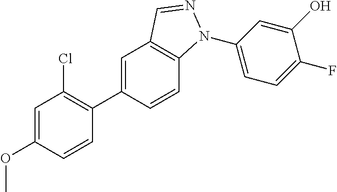 [SMILES: COc1ccc(-c2ccc3c(cnn3-c3ccc(F)c(O)c3)c2)c(Cl)c1] | 5-(5-(2-Chloro-4-methoxyphenyl)-1H-indazol- 1-yl)-2-fluorophenol |
| 5.04 | 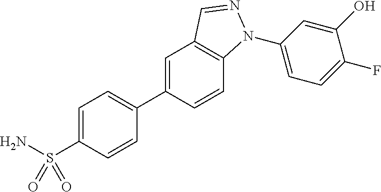 [SMILES: NS(=O)(=O)c1ccc(-c2ccc3c(cnn3-c3ccc(F)c(O)c3)c2)cc1] | 4-(1-(4-Fluoro-3-hydroxyphenyl)-1H-indazol-5- yl)benzenesulfonamide |
| 5.05 | 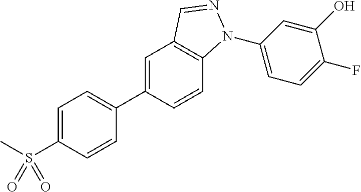 [SMILES: CS(=O)(=O)c1ccc(-c2ccc3c(cnn3-c3ccc(F)c(O)c3)c2)cc1] | 2-Fluoro-5-(5-(4-(methylsulfonyl)phenyl)-1H- indazol-1-yl)phenol |
| 5.06 | 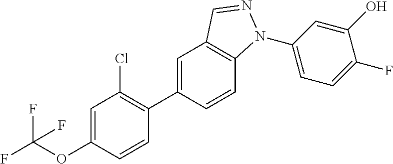 [SMILES: Oc1cc(-n2ncc3cc(-c4ccc(OC(F)(F)F)cc4Cl)ccc32)ccc1F] | 5-(5-(2-Chloro-4-(trifluoromethoxy)phenyl)- 1H-indazol-1-yl)-2-fluorophenol |
| 5.07 | 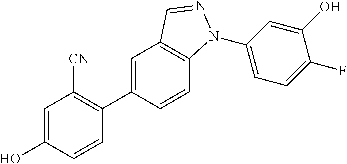 [SMILES: N#Cc1cc(O)ccc1-c1ccc2c(cnn2-c2ccc(F)c(O)c2)c1] | 2-(1-(4-Fluoro-3-hydroxyphenyl)-1H-indazol-5- yl)-5-hydroxybenzonitrile |
| 5.08 | 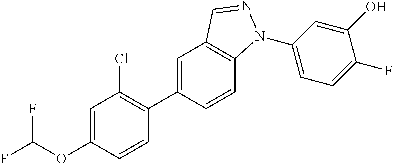 [SMILES: Oc1cc(-n2ncc3cc(-c4ccc(OC(F)F)cc4Cl)ccc32)ccc1F] | 5-(5-(2-Chloro-4-(difluoromethoxy)phenyl)-1H- indazol-1-yl)-2-fluorophenol |
| 5.09 | 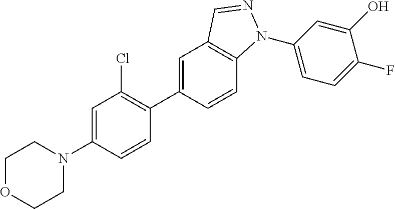 [SMILES: Oc1cc(-n2ncc3cc(-c4ccc(N5CCOCC5)cc4Cl)ccc32)ccc1F] | 5-(5-(2-Chloro-4-morpholinophenyl)-1H- indazol-1-yl)-2-fluorophenol |
| 5.10 | 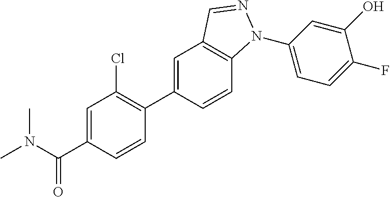 [SMILES: CN(C)C(=O)c1ccc(-c2ccc3c(cnn3-c3ccc(F)c(O)c3)c2)c(Cl)c1] | 3-Chloro-4-(1-(4-fluoro-3-hydroxyphenyl)-1H- indazol-5-yl)-N,N-dimethylbenzamide |
| 5.11 | 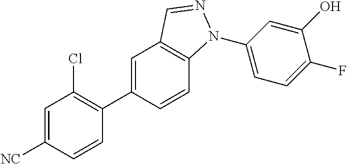 [SMILES: N#Cc1ccc(-c2ccc3c(cnn3-c3ccc(F)c(O)c3)c2)c(Cl)c1] | 3-Chloro-4-(1-(4-fluoro-3-hydroxyphenyl)-1H- indazol-5-yl)benzonitrile |
| 5.12 | 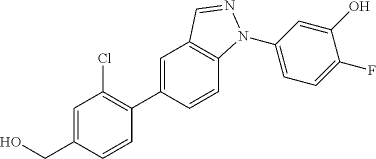 [SMILES: OCc1ccc(-c2ccc3c(cnn3-c3ccc(F)c(O)c3)c2)c(Cl)c1] | 5-(5-(2-Chloro-4-(hydroxymethyl)phenyl)-1H- indazol-1-yl)-2-fluorophenol |
| 5.13 | 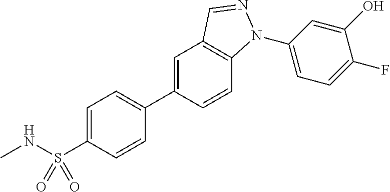 [SMILES: CNS(=O)(=O)c1ccc(-c2ccc3c(cnn3-c3ccc(F)c(O)c3)c2)cc1] | 4-(1-(4-Fluoro-3-hydroxyphenyl)-1H-indazol-5- yl)-N-methylbenzenesulfonamide |
| 5.14 | 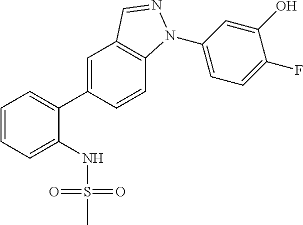 [SMILES: CS(=O)(=O)Nc1ccccc1-c1ccc2c(cnn2-c2ccc(F)c(O)c2)c1] | N-(2-(1-(4-Fluoro-3-hydroxyphenyl)-1H- indazol-5-yl)phenyl)methanesulfonamide |
| 5.15 | 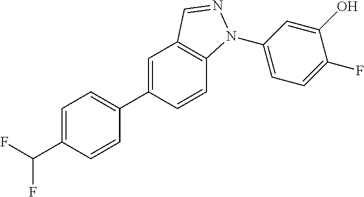 [SMILES: Oc1cc(-n2ncc3cc(-c4ccc(C(F)F)cc4)ccc32)ccc1F] | 5-(5-(4-(Difluoromethyl)phenyl)-1H-indazol-1- yl)-2-fluorophenol |
| 5.16 | 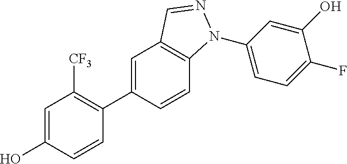 [SMILES: Oc1ccc(-c2ccc3c(cnn3-c3ccc(F)c(O)c3)c2)c(C(F)(F)F)c1] | 2-Fluoro-5-(5-(4-hydroxy-2- (trifluoromethyl)phenyl)-1H-indazol-1- yl)phenol |
| 5.17 | 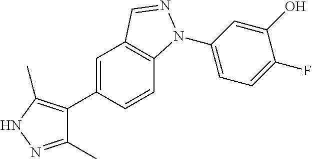 [SMILES: Cc1n[nH]c(C)c1-c1ccc2c(cnn2-c2ccc(F)c(O)c2)c1] | 5-(5-(3,5-Dimethyl-1H-pyrazol-4-yl)-1H- indazol-1-yl)-2-fluorophenol |
| 5.18 | 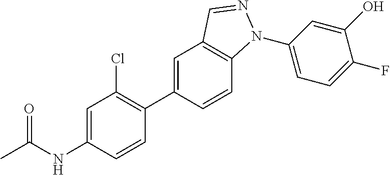 [SMILES: CC(=O)Nc1ccc(-c2ccc3c(cnn3-c3ccc(F)c(O)c3)c2)c(Cl)c1] | N-(3-Chloro-4-(1-(4-fluoro-3-hydroxyphenyl)- 1H-indazol-5-yl)phenyl)acetamide |
| 5.19 | 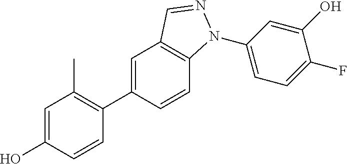 [SMILES: Cc1cc(O)ccc1-c1ccc2c(cnn2-c2ccc(F)c(O)c2)c1] | 2-Fluoro-5-(5-(4-hydroxy-2-methylphenyl)-1H- indazol-1-yl)phenol |
| 5.20 | 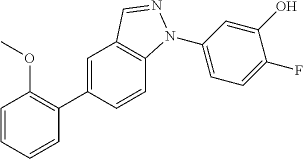 [SMILES: COc1ccccc1-c1ccc2c(cnn2-c2ccc(F)c(O)c2)c1] | 2-Fluoro-5-(5-(2-methoxyphenyl)-1H-indazol- 1-yl)phenol |
| 5.21 | 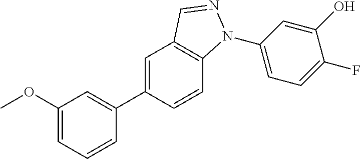 [SMILES: COc1cccc(-c2ccc3c(cnn3-c3ccc(F)c(O)c3)c2)c1] | 2-Fluoro-5-(5-(3-methoxyphenyl)-1H-indazol- 1-yl)phenol |
| 5.22 | 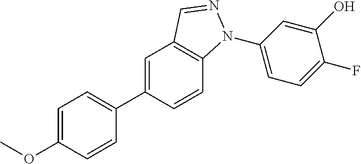 [SMILES: COc1ccc(-c2ccc3c(cnn3-c3ccc(F)c(O)c3)c2)cc1] | 2-Fluoro-5-(5-(4-methoxyphenyl)-1H-indazol- 1-yl)phenol |
| 5.23 | 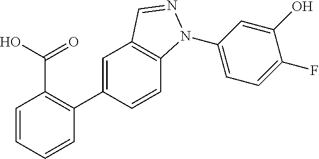 [SMILES: O=C(O)c1ccccc1-c1ccc2c(cnn2-c2ccc(F)c(O)c2)c1] | 2-(1-(4-Fluoro-3-hydroxyphenyl)-1H-indazol-5- yl)benzoic acid |
| 5.24 | 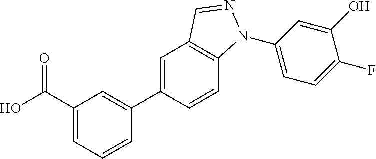 [SMILES: O=C(O)c1cccc(-c2ccc3c(cnn3-c3ccc(F)c(O)c3)c2)c1] | 3-(1-(4-Fluoro-3-hydroxyphenyl)-1H-indazol-5- yl)benzoic acid |
| 5.25 | 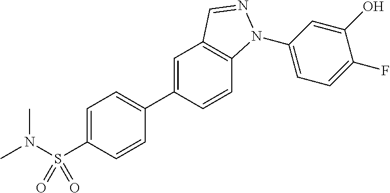 [SMILES: CN(C)S(=O)(=O)c1ccc(-c2ccc3c(cnn3-c3ccc(F)c(O)c3)c2)cc1] | 4-(1-(4-Fluoro-3-hydroxyphenyl)-1H-indazol-5- yl)-N,N-dimethylbenzenesulfonamide |
| 5.26 | 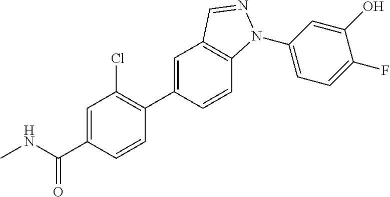 [SMILES: CNC(=O)c1ccc(-c2ccc3c(cnn3-c3ccc(F)c(O)c3)c2)c(Cl)c1] | 3-Chloro-4-(1-(4-fluoro-3-hydroxyphenyl)-1H- indazol-5-yl)-N-methylbenzamide |
| 5.27 | 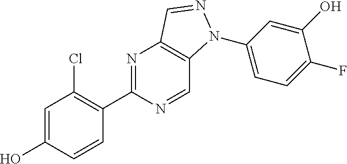 [SMILES: Oc1ccc(-c2ncc3c(cnn3-c3ccc(F)c(O)c3)n2)c(Cl)c1] | 5-(5-(2-Chloro-4-hydroxyphenyl)-1H- pyrazolo[4,3-d]pyrimidin-1-yl)-2-fluorophenol |
| 5.28 | 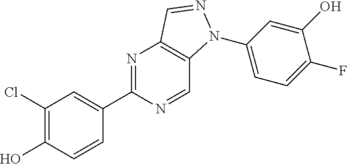 [SMILES: Oc1cc(-n2ncc3nc(-c4ccc(O)c(Cl)c4)ncc32)ccc1F] | 2-Chloro-4-(1-(4-fluoro-3-hydroxyphenyl)-1H- pyrazolo[4,3-d]pyrimidin-5-yl)phenol |
| 5.29 | 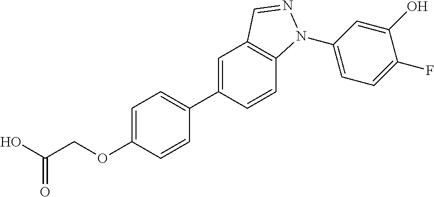 [SMILES: O=C(O)COc1ccc(-c2ccc3c(cnn3-c3ccc(F)c(O)c3)c2)cc1] | 2-(4-(1-(4-Fluoro-3-hydroxyphenyl)-1H- indazol-5-yl)phenoxy)acetic acid |
| 5.30 | 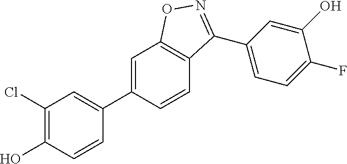 [SMILES: Oc1cc(-c2noc3cc(-c4ccc(O)c(Cl)c4)ccc23)ccc1F] | 2-Chloro-4-(3-(4-fluoro-3- hydroxyphenyl)benzo[d]isoxazol-6-yl)phenol |
| 6 | 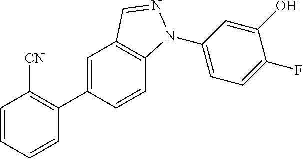 [SMILES: N#Cc1ccccc1-c1ccc2c(cnn2-c2ccc(F)c(O)c2)c1] | 2-(1-(4-Fluoro-3-hydroxyphenyl)-1H-indazol-5- yl)benzonitrile |
| 6.01 | 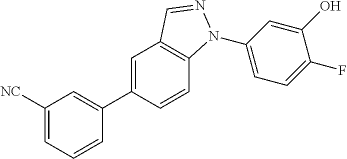 [SMILES: N#Cc1cccc(-c2ccc3c(cnn3-c3ccc(F)c(O)c3)c2)c1] | 3-(1-(4-Fluoro-3-hydroxyphenyl)-1H-indazol-5- yl)benzonitrile |
| 7 | 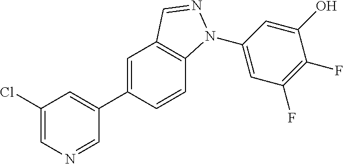 [SMILES: Oc1cc(-n2ncc3cc(-c4cncc(Cl)c4)ccc32)cc(F)c1F] | 5-(5-(5-Chloropyridin-3-yl)-1H-indazol-1-yl)- 2,3-difluorophenol |
| 7.01 | 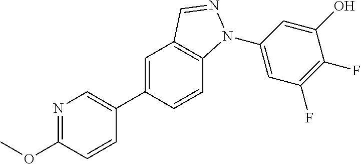 [SMILES: COc1ccc(-c2ccc3c(cnn3-c3cc(O)c(F)c(F)c3)c2)cn1] | 2,3-Difluoro-5-(5-(6-methoxypyridin-3-yl)-1H- indazol-1-yl)phenol |
| 7.02 | 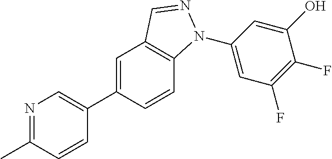 [SMILES: Cc1ccc(-c2ccc3c(cnn3-c3cc(O)c(F)c(F)c3)c2)cn1] | 2,3-Difluoro-5-(5-(6-methylpyridin-3-yl)-1H- indazol-1-yl)phenol |
| 7.03 | 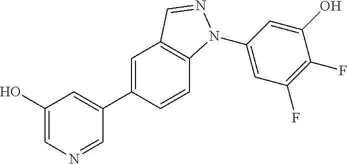 [SMILES: Oc1cncc(-c2ccc3c(cnn3-c3cc(O)c(F)c(F)c3)c2)c1] | 5-(1-(3,4-Difluoro-5-hydroxyphenyl)-1H- indazol-5-yl)pyridine-3-ol |
| 7.04 | 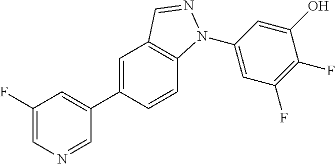 [SMILES: Oc1cc(-n2ncc3cc(-c4cncc(F)c4)ccc32)cc(F)c1F] | 2,3-Difluoro-5-(5-(5-fluoropyridin-3-yl)-1H- indazol-1-yl)phenol |
| 8 | 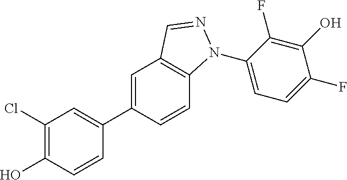 [SMILES: Oc1ccc(-c2ccc3c(cnn3-c3ccc(F)c(O)c3F)c2)cc1Cl] | 3-(5-(3-Chloro-4-hydroxyphenyl)-1H-indazol- 1-yl)-2,6-difluorophenol |
| 8.01 | 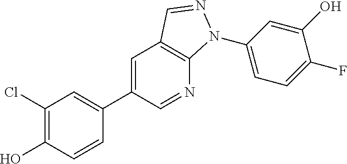 [SMILES: Oc1cc(-n2ncc3cc(-c4ccc(O)c(Cl)c4)cnc32)ccc1F] | 2-Chloro-4-(1-(4-fluoro-3-hydroxyphenyl)-1H- pyrazolo[3,4-b]pyridine-5-yl)phenol |
| 8.02 | 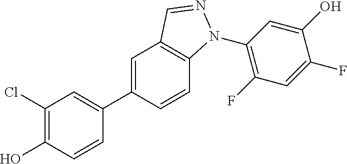 [SMILES: Oc1cc(-n2ncc3cc(-c4ccc(O)c(Cl)c4)ccc32)c(F)cc1F] | 5-(5-(3-Chloro-4-hydroxyphenyl)-1H-indazol- 1-yl)-2,4-difluorophenol |
| 8.03 | 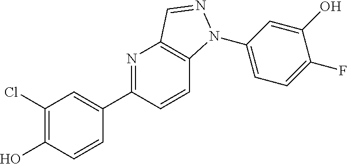 [SMILES: Oc1cc(-n2ncc3nc(-c4ccc(O)c(Cl)c4)ccc32)ccc1F] | 2-Chloro-4-(1-(4-fluoro-3-hydroxyphenyl)-1H- pyrazolo[4,3-b]pyridine-5-yl)phenol |
| 9 | 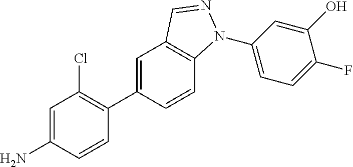 [SMILES: Nc1ccc(-c2ccc3c(cnn3-c3ccc(F)c(O)c3)c2)c(Cl)c1] | 5-(5-(4-Amino-2-chlorophenyl)-1H-indazol-1- yl)-2-fluorophenol |
| 10 | 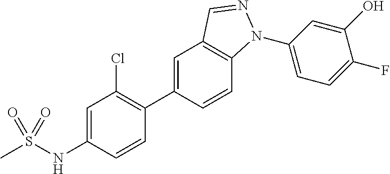 [SMILES: CS(=O)(=O)Nc1ccc(-c2ccc3c(cnn3-c3ccc(F)c(O)c3)c2)c(Cl)c1] | N-(3-Chloro-4-(1-(4-fluoro-3-hydroxyphenyl)- 1H-indazol-5-yl)phenyl)methanesulfonamide |
| 11 | 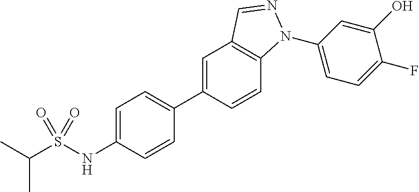 [SMILES: CC(C)S(=O)(=O)Nc1ccc(-c2ccc3c(cnn3-c3ccc(F)c(O)c3)c2)cc1] | N-(4-(1-(4-Fluoro-3-hydroxyphenyl)-1H- indazol-5-yl)phenyl)propane-2-sulfonamide |
| 11.01 | 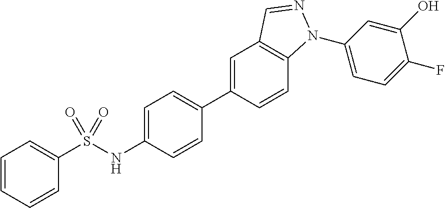 [SMILES: O=S(=O)(Nc1ccc(-c2ccc3c(cnn3-c3ccc(F)c(O)c3)c2)cc1)c1ccccc1] | N-(4-(1-(4-Fluoro-3-hydroxyphenyl)-1H- indazol-5-yl)phenyl)benzenesulfonamide |
| 11.02 | 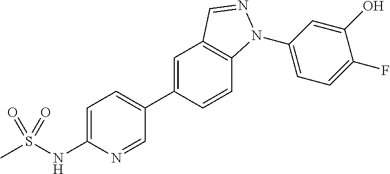 [SMILES: CS(=O)(=O)Nc1ccc(-c2ccc3c(cnn3-c3ccc(F)c(O)c3)c2)cn1] | N-(5-(1-(4-Fluoro-3-hydroxyphenyl)-1H- indazol-5-yl)pyridine-2-yl)methanesulfonamide |
| 12 | 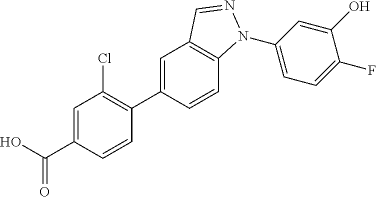 [SMILES: O=C(O)c1ccc(-c2ccc3c(cnn3-c3ccc(F)c(O)c3)c2)c(Cl)c1] | 3-Chloro-4-(1-(4-Fluoro-3-hydroxyphenyl)-1H- indazol-5-yl)benzoic acid-Chloro-4-(1-(4- Fluoro-3-hydroxyphenyl)-1H-indazol-5- yl)benzoic acid |
| 13 | 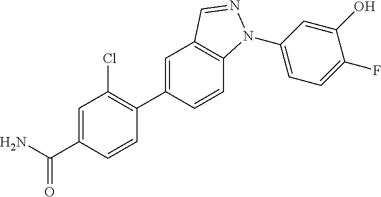 [SMILES: NC(=O)c1ccc(-c2ccc3c(cnn3-c3ccc(F)c(O)c3)c2)c(Cl)c1] | 3-Chloro-4-(1-(4-fluoro-3-hydroxyphenyl)-1H- indazol-5-yl)benzamide |
| 14 | 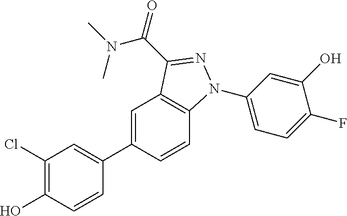 [SMILES: CN(C)C(=O)c1nn(-c2ccc(F)c(O)c2)c2ccc(-c3ccc(O)c(Cl)c3)cc12] | 5-(3-Chloro-4-hydroxyphenyl)-1-(4-fluoro-3- hydroxyphenyl)-N,N-dimethyl-1H-indazole-3- carboxamide |
| 15 | 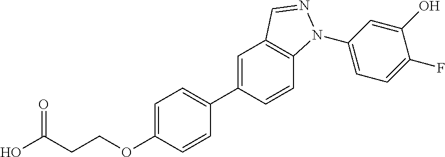 [SMILES: O=C(O)CCOc1ccc(-c2ccc3c(cnn3-c3ccc(F)c(O)c3)c2)cc1] | 3-(4-(1-(4-Fluoro-3-hydroxyphenyl)-1H- indazol-5-yl)phenoxy)propanoic acid |
| 16 | 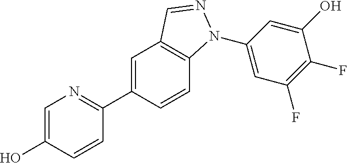 [SMILES: Oc1ccc(-c2ccc3c(cnn3-c3cc(O)c(F)c(F)c3)c2)nc1] | 6-(1-(3,4-Difluoro-5-hydroxyphenyl)-1H- indazol-5-yl)pyridine-3-ol |
| 16.01 | 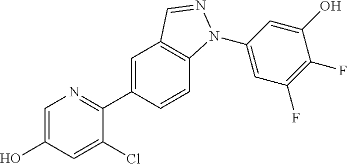 [SMILES: Oc1cnc(-c2ccc3c(cnn3-c3cc(O)c(F)c(F)c3)c2)c(Cl)c1] | 5-Chloro-6-(1-(3,4-difluoro-5-hydroxyphenyl)- 1H-indazol-5-yl)pyridine-3-ol |
| 16.02 | 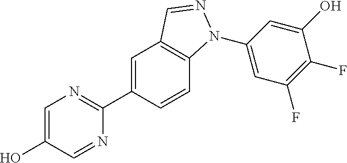 [SMILES: Oc1cnc(-c2ccc3c(cnn3-c3cc(O)c(F)c(F)c3)c2)nc1] | 2-(1-(3,4-Difluoro-5-hydroxyphenyl)-1H- indazol-5-yl)pyrimidin-5-ol |
| 16.03 | 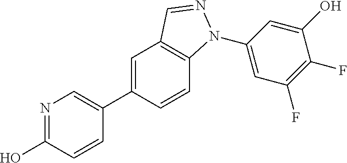 [SMILES: Oc1ccc(-c2ccc3c(cnn3-c3cc(O)c(F)c(F)c3)c2)cn1] | 5-(1-(3,4-difluoro-5-hydroxyphenyl)-1H- indazol-5-yl)pyridine-2-ol |
| 17 | 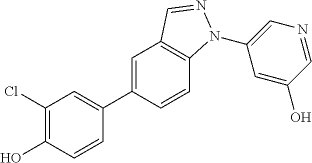 [SMILES: Oc1cncc(-n2ncc3cc(-c4ccc(O)c(Cl)c4)ccc32)c1] | 5-(5-(3-Chloro-4-hydroxyphenyl)-1H-indazol- 1-yl)pyridin-3-ol |
| 18 | 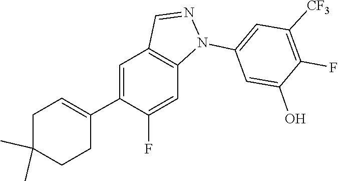 [SMILES: CC1(C)CC=C(c2cc3cnn(-c4cc(O)c(F)c(C(F)(F)F)c4)c3cc2F)CC1] | 5-(5-(4,4-Dimethylcyclohex-1-en-1-yl)-6- fluoro-1H-indazol-1-yl)-2-fluoro-3- (trifluoromethyl)phenol |
| 18.01 | 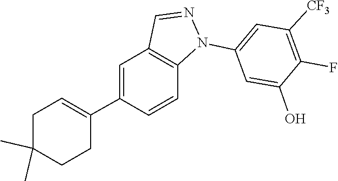 [SMILES: CC1(C)CC=C(c2ccc3c(cnn3-c3cc(O)c(F)c(C(F)(F)F)c3)c2)CC1] | 5-(5-(4,4-dimethylcyclohex-1-en-1-yl)-1H- indazol-1-yl)-2-fluoro-3- (trifluoromethyl)phenol |
| 18.02 | 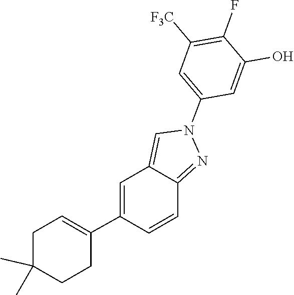 [SMILES: CC1(C)CC=C(c2ccc3nn(-c4cc(O)c(F)c(C(F)(F)F)c4)cc3c2)CC1] | 5-(5-(4,4-Dimethylcyclohex-1-en-1-yl)-2H- indazol-2-yl)-2-fluoro-3- (trifluoromethyl)phenol |
| 19 | 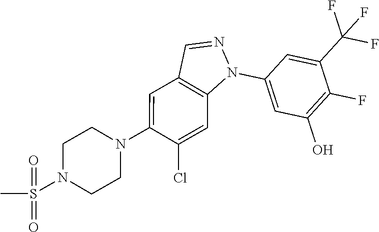 [SMILES: CS(=O)(=O)N1CCN(c2cc3cnn(-c4cc(O)c(F)c(C(F)(F)F)c4)c3cc2Cl)CC1] | 5-(6-Chloro-5-(4-(methylsulfonyl)piperazin-1- yl)-1H-indazol-1-yl)-2-fluoro-3- (trifluoromethyl)phenol |
| 19.01 | 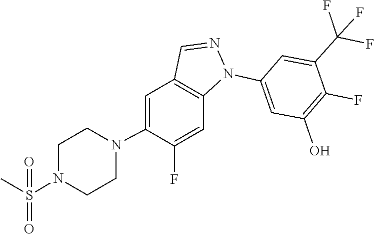 [SMILES: CS(=O)(=O)N1CCN(c2cc3cnn(-c4cc(O)c(F)c(C(F)(F)F)c4)c3cc2F)CC1] | 2-Fluoro-5-(6-fluoro-5-(4- (methylsulfonyl)piperazin-1-yl)-1H-indazol-1- yl)-3-(trifluoromethyl)phenol |
| 19.02 | 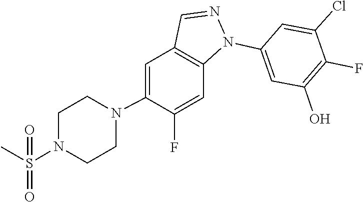 [SMILES: CS(=O)(=O)N1CCN(c2cc3cnn(-c4cc(O)c(F)c(Cl)c4)c3cc2F)CC1] | 3-Chloro-2-fluoro-5-(6-fluoro-5-(4- (methylsulfonyl)piperazin-1-yl)-1H-indazol-1- yl)phenol |
| 19.03 | 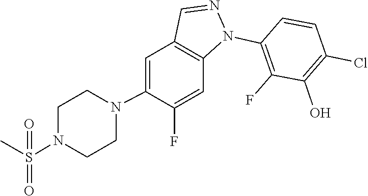 [SMILES: CS(=O)(=O)N1CCN(c2cc3cnn(-c4ccc(Cl)c(O)c4F)c3cc2F)CC1] | 6-Chloro-2-fluoro-3-(6-fluoro-5-(4- (methylsulfonyl)piperazin-1-yl)-1H-indazol-1- yl)phenol |
| 19.04 | 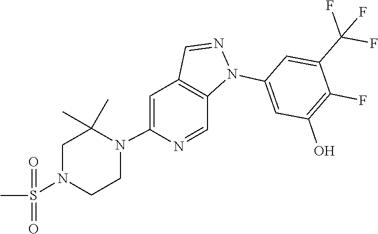 [SMILES: CC1(C)CN(S(C)(=O)=O)CCN1c1cc2cnn(-c3cc(O)c(F)c(C(F)(F)F)c3)c2cn1] | 5-(5-(2,2-Dimethyl-4- (methylsulfonyl)piperazin-1-yl)-1H- pyrazolo[3,4-c]pyridine-1-yl)-2-fluoro-3- (trifluoromethyl)phenol |
| 19.05 | 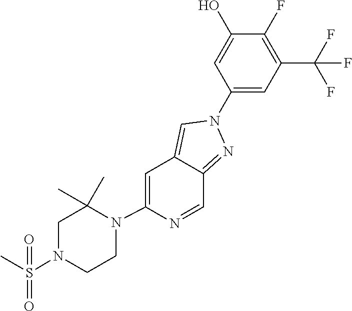 [SMILES: CC1(C)CN(S(C)(=O)=O)CCN1c1cc2cn(-c3cc(O)c(F)c(C(F)(F)F)c3)nc2cn1] | 5-(5-(2,2-Dimethyl-4- (methylsulfonyl)piperazin-1-yl)-2H- pyrazolo[3,4-c]pyridine-2-yl)-2-fluoro-3- (trifluoromethyl)phenol |
| 19.06 | 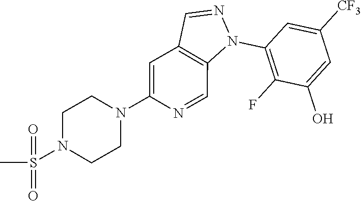 [SMILES: CS(=O)(=O)N1CCN(c2cc3cnn(-c4cc(C(F)(F)F)cc(O)c4F)c3cn2)CC1] | 2-Fluoro-3-(5-(4-(methylsulfonyl)piperazin-1- yl)-1H-pyrazolo[3,4-c]pyridine-1-yl)-5- (trifluoromethyl)phenol |
| 19.07 | 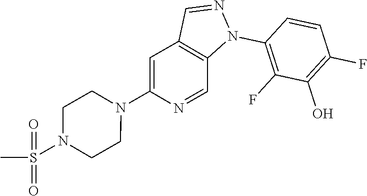 [SMILES: CS(=O)(=O)N1CCN(c2cc3cnn(-c4ccc(F)c(O)c4F)c3cn2)CC1] | 2,6-Difluoro-3-(5-(4-(methylsulfonyl)piperazin- 1-yl)-1H-pyrazolo[3,4-c]pyridine-1-yl)phenol |
| 19.08 | 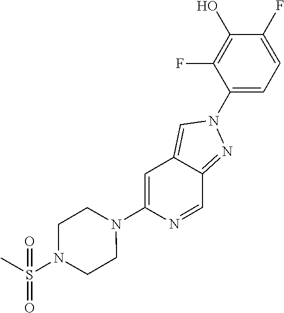 [SMILES: CS(=O)(=O)N1CCN(c2cc3cn(-c4ccc(F)c(O)c4F)nc3cn2)CC1] | 2,6-Difluoro-3-(5-(4-(methylsulfonyl)piperazin- 1-yl)-2H-pyrazolo[3,4-c]pyridine-2-yl)phenol |
| 19.09 | 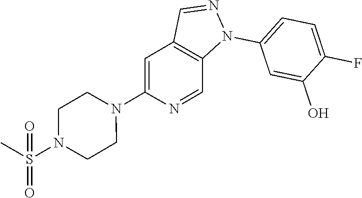 [SMILES: CS(=O)(=O)N1CCN(c2cc3cnn(-c4ccc(F)c(O)c4)c3cn2)CC1] | 2-Fluoro-5-(5-(4-(methylsulfonyl)piperazin-1- yl)-1H-pyrazolo[3,4-c]pyridine-1-yl)phenol |
| 19.10 | 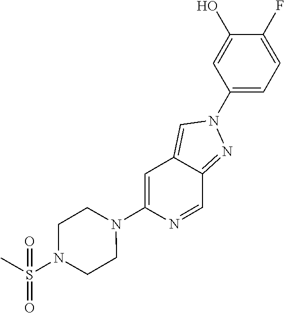 [SMILES: CS(=O)(=O)N1CCN(c2cc3cn(-c4ccc(F)c(O)c4)nc3cn2)CC1] | 2-Fluoro-5-(5-(4-(methylsulfonyl)piperazin-1- yl)-2H-pyrazolo[3,4-c]pyridine-2-yl)phenol |
| 19.11 | 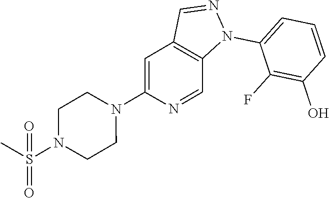 [SMILES: CS(=O)(=O)N1CCN(c2cc3cnn(-c4cccc(O)c4F)c3cn2)CC1] | 2-Fluoro-3-(5-(4-(methylsulfonyl)piperazin-1- yl)-1H-pyrazolo[3,4-c]pyridine-1-yl)phenol |
| 19.12 | 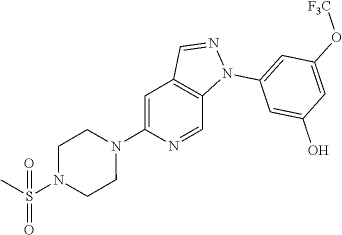 [SMILES: CS(=O)(=O)N1CCN(c2cc3cnn(-c4cc(O)cc(OC(F)(F)F)c4)c3cn2)CC1] | 3-(5-(4-(Methylsulfonyl)piperazin-1-yl)-1H- pyrazolo[3,4-c]pyridine-1-yl)-5- (trifluoromethoxy)phenol |
| 19.13 | 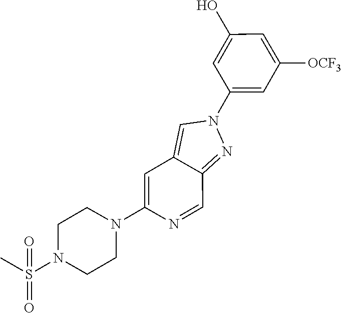 [SMILES: CS(=O)(=O)N1CCN(c2cc3cn(-c4cc(O)cc(OC(F)(F)F)c4)nc3cn2)CC1] | 3-(5-(4-(Methylsulfonyl)piperazin-1-yl)-2H- pyrazolo[3,4-c]pyridin-2-yl)-5- (trifluoromethoxy)phenol |
| 19.14 | 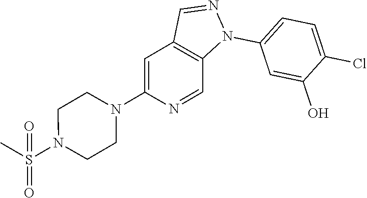 [SMILES: CS(=O)(=O)N1CCN(c2cc3cnn(-c4ccc(Cl)c(O)c4)c3cn2)CC1] | 2-Chloro-5-(5-(4-(methylsulfonyl)piperazin-1- yl)-1H-pyrazolo[3,4-c]pyridine-1-yl)phenol |
| 19.15 | 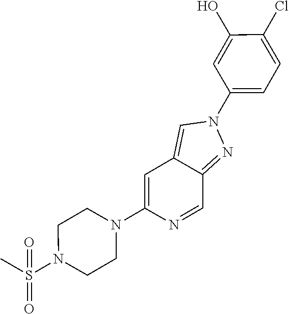 [SMILES: CS(=O)(=O)N1CCN(c2cc3cn(-c4ccc(Cl)c(O)c4)nc3cn2)CC1] | 2-Chloro-5-(5-(4-(methylsulfonyl)piperazin-1- yl)-2H-pyrazolo[3,4-c]pyridine-2-yl)phenol |
| 19.16 | 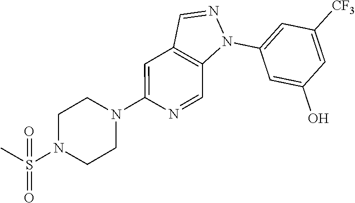 [SMILES: CS(=O)(=O)N1CCN(c2cc3cnn(-c4cc(O)cc(C(F)(F)F)c4)c3cn2)CC1] | 3-(5-(4-(Methylsulfonyl)piperazin-1-yl)-1H- pyrazolo[3,4-c]pyridine-1-yl)-5- (trifluoromethyl)phenol |
| 19.17 | 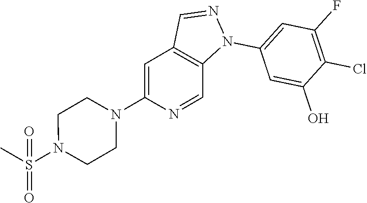 [SMILES: CS(=O)(=O)N1CCN(c2cc3cnn(-c4cc(O)c(Cl)c(F)c4)c3cn2)CC1] | 2-Chloro-3-fluoro-5-(5-(4- (methylsulfonyl)piperazin-1-yl)-1H- pyrazolo[3,4-c]pyridine-1-yl)phenol |
| 19.18 | 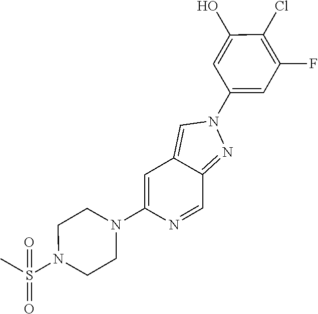 [SMILES: CS(=O)(=O)N1CCN(c2cc3cn(-c4cc(O)c(Cl)c(F)c4)nc3cn2)CC1] | 2-Chloro-3-fluoro-5-(5-(4- (methylsulfonyl)piperazin-1-yl)-2H- pyrazolo[3,4-c]pyridine-2-yl)phenol |
| 19.19 | 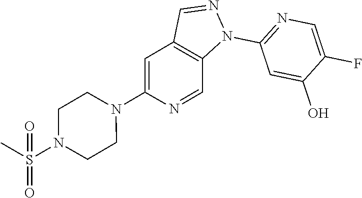 [SMILES: CS(=O)(=O)N1CCN(c2cc3cnn(-c4cc(O)c(F)cn4)c3cn2)CC1] | 5-Fluoro-2-(5-(4-(methylsulfonyl)piperazin-1- yl)-1H-pyrazolo[3,4-c]pyridine-1-yl)pyridine-4- ol |
| 19.20 | 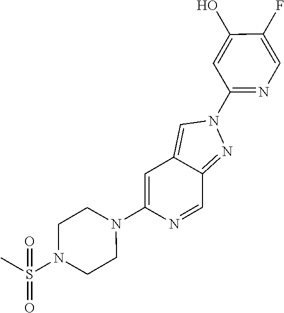 [SMILES: CS(=O)(=O)N1CCN(c2cc3cn(-c4cc(O)c(F)cn4)nc3cn2)CC1] | 5-Fluoro-2-(5-(4-(methylsulfonyl)piperazin-1- yl)-2H-pyrazolo[3,4-c]pyridine-2-yl)pyridine-4- ol |
| 19.21 | 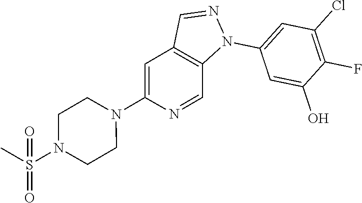 [SMILES: CS(=O)(=O)N1CCN(c2cc3cnn(-c4cc(O)c(F)c(Cl)c4)c3cn2)CC1] | 3-Chloro-2-fluoro-5-(5-(4- (methylsulfonyl)piperazin-1-yl)-1H- pyrazolo[3,4-c]pyridine-1-yl)phenol |
| 19.22 | 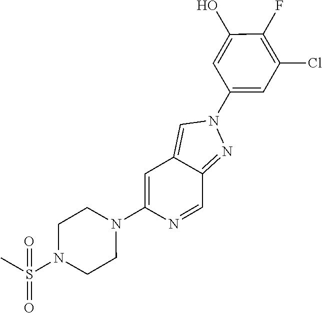 [SMILES: CS(=O)(=O)N1CCN(c2cc3cn(-c4cc(O)c(F)c(Cl)c4)nc3cn2)CC1] | 3-Chloro-2-fluoro-5-(5-(4- (methylsulfonyl)piperazin-1-yl)-2H- pyrazolo[3,4-c]pyridine-2-yl)phenol |
| 19.23 | 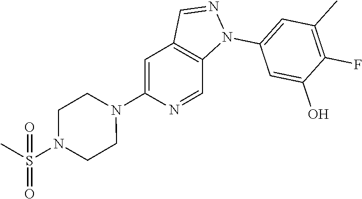 [SMILES: Cc1cc(-n2ncc3cc(N4CCN(S(C)(=O)=O)CC4)ncc32)cc(O)c1F] | 2-Fluoro-3-methyl-5-(5-(4- (methylsulfonyl)piperazin-1-yl)-1H- pyrazolo[3,4-c]pyridine-1-yl)phenol |
| 19.24 | 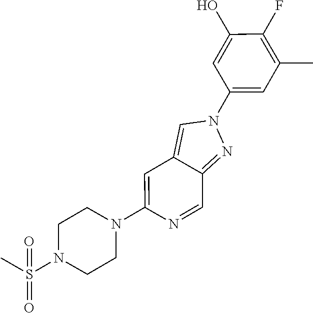 | 2-Fluoro-3-methyl-5-(5-(4- (methylsulfonyl)piperazin-1-yl)-2H- pyrazolo[3,4-c]pyridine-2-yl)phenol |
| 19.25 | 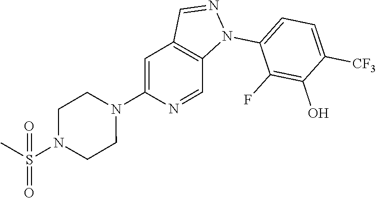 [SMILES: CS(=O)(=O)N1CCN(c2cc3cnn(-c4ccc(C(F)(F)F)c(O)c4F)c3cn2)CC1] | 2-Fluoro-3-(5-(4-(methylsulfonyl)piperazin-1- yl)-1H-pyrazolo[3,4-c]pyridine-1-yl)-6- (trifluoromethyl)phenol |
| 19.26 | 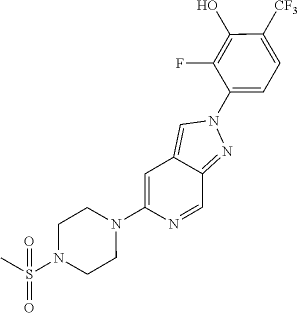 [SMILES: CS(=O)(=O)N1CCN(c2cc3cn(-c4ccc(C(F)(F)F)c(O)c4F)nc3cn2)CC1] | 2-Fluoro-3-(5-(4-(methylsulfony])piperazin-1- yl)-2H-pyrazolo[3,4-c]pyridine-2-yl)-6- (trifluoromethyl)phenol |
| 19.27 | 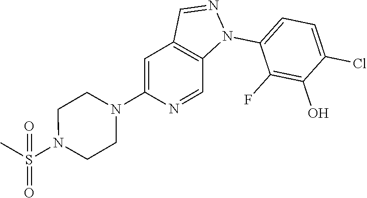 [SMILES: CS(=O)(=O)N1CCN(c2cc3cnn(-c4ccc(Cl)c(O)c4F)c3cn2)CC1] | 6-Chloro-2-fluoro-3-(5-(4- (methylsulfonyl)piperazin-1-yl)-1H- pyrazolo[3,4-c]pyridine-1-yl)phenol |
| 19.28 | 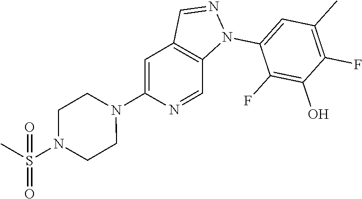 [SMILES: Cc1cc(-n2ncc3cc(N4CCN(S(C)(=O)=O)CC4)ncc32)c(F)c(O)c1F] | 2,6-Difluoro-3-methyl-5-(5-(4- (methylsulfonyl)piperazin-1-yl)-1H- pyrazolo[3,4-c]pyridine-1-yl)phenol |
| 19.29 | 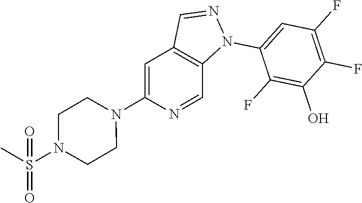 [SMILES: CS(=O)(=O)N1CCN(c2cc3cnn(-c4cc(F)c(F)c(O)c4F)c3cn2)CC1] | 2,3,6-Trifluoro-5-(5-(4- (methylsulfonyl)piperazin-1-yl)-1H- pyrazolo[3,4-c]pyridine-1-yl)phenol |
| 19.30 | 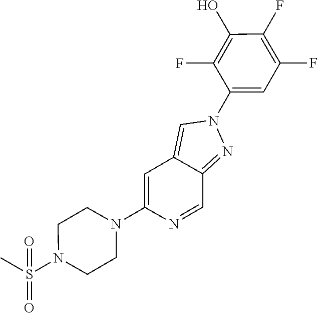 [SMILES: CS(=O)(=O)N1CCN(c2cc3cn(-c4cc(F)c(F)c(O)c4F)nc3cn2)CC1] | 2,3,6-Trifluoro-5-(5-(4- (methylsulfonyl)piperazin-1-yl)-2H- pyrazolo[3,4-c]pyridine-2-yl)phenol |
| 19.31 | 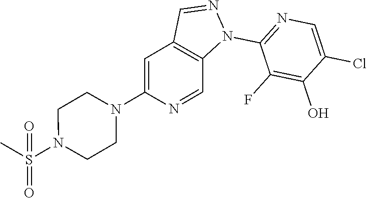 [SMILES: CS(=O)(=O)N1CCN(c2cc3cnn(-c4ncc(Cl)c(O)c4F)c3cn2)CC1] | 5-Chloro-3-fluoro-2-(5-(4- (methylsulfonyl)piperazin-1-yl)-1H- pyrazolo[3,4-c]pyridine-1-yl)pyridine-4-ol |
| 19.32 | 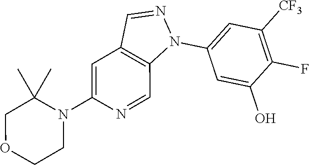 [SMILES: CC1(C)COCCN1c1cc2cnn(-c3cc(O)c(F)c(C(F)(F)F)c3)c2cn1] | 5-(5-(3,3-Dimethylmorpholino)-1H- pyrazolo[3,4-c]pyridine-1-yl)-2-fluoro-3- (trifluoromethyl)phenol |
| 19.33 | 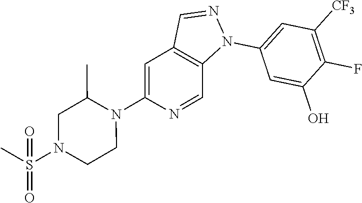 [SMILES: CC1CN(S(C)(=O)=O)CCN1c1cc2cnn(-c3cc(O)c(F)c(C(F)(F)F)c3)c2cn1] | 2-Fluoro-5-(5-(2-methyl-4- (methylsulfonyl)piperazin-1-yl)-1H- pyrazolo[3,4-c]pyridine-1-yl)-3- (trifluoromethyl)phenol |
| 19.34 | 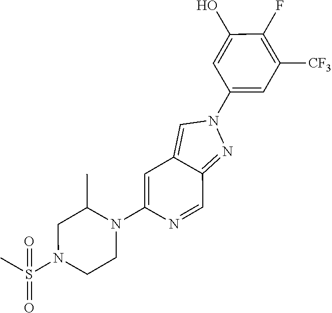 [SMILES: CC1CN(S(C)(=O)=O)CCN1c1cc2cn(-c3cc(O)c(F)c(C(F)(F)F)c3)nc2cn1] | 2-Fluoro-5-(5-(2-methyl-4- (methylsulfonyl)piperazin-1-yl)-2H- pyrazolo[3,4-c]pyridine-2-yl)-3- (trifluoromethyl)phenol |
| 19.35 | 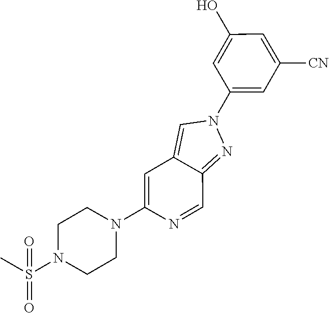 [SMILES: CS(=O)(=O)N1CCN(c2cc3cn(-c4cc(O)cc(C#N)c4)nc3cn2)CC1] | 3-Hydroxy-5-(5-(4-(methylsulfonyl)piperazin- 1-yl)-2H-pyrazolo[3,4-c]pyridine-2- yl)benzonitrile |
| 19.36 | 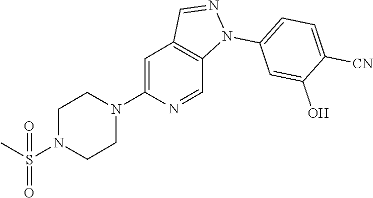 [SMILES: CS(=O)(=O)N1CCN(c2cc3cnn(-c4ccc(C#N)c(O)c4)c3cn2)CC1] | 2-Hydroxy-4-(5-(4-(methylsulfonyl)piperazin- 1-yl)-1H-pyrazolo[3,4-c]pyridine-1- yl)benzonitrile |
| 19.37 | 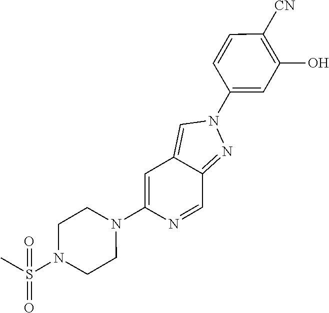 [SMILES: CS(=O)(=O)N1CCN(c2cc3cn(-c4ccc(C#N)c(O)c4)nc3cn2)CC1] | 2-Hydroxy-4-(5-(4-(methylsulfonyl)piperazin- 1-yl)-2H-pyrazolo[3,4-c]Pyridine-2- yl)benzonitrile |
| 19.38 | 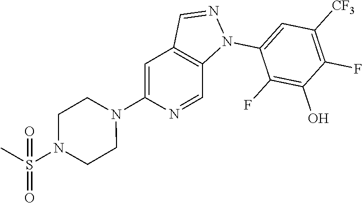 [SMILES: CS(=O)(=O)N1CCN(c2cc3cnn(-c4cc(C(F)(F)F)c(F)c(O)c4F)c3cn2)CC1] | 2,6-Difluoro-3-(5-(4-(methylsulfonyl)piperazin- 1-yl)-1H-pyrazolo[3,4-c]pyridine-1-yl)-5- (trifluoromethyl)phenol |
| 19.39 | 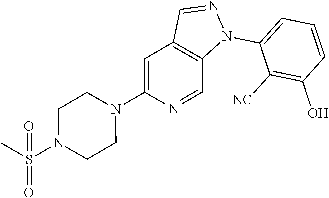 [SMILES: CS(=O)(=O)N1CCN(c2cc3cnn(-c4cccc(O)c4C#N)c3cn2)CC1] | 2-Hydroxy-6-(5-(4-(methylsulfonyl)piperazin- 1-yl)-1H-pyrazolo[3,4-c]pyridine-1- yl)benzonitrile |
| 19.40 | 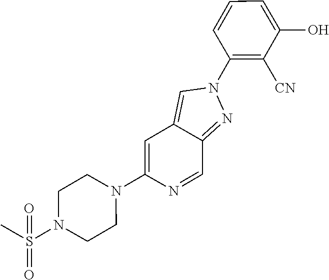 [SMILES: CS(=O)(=O)N1CCN(c2cc3cn(-c4cccc(O)c4C#N)nc3cn2)CC1] | 2-Hydroxy-6-(5-(4-(methylsulfonyl)piperazin- 1-yl)-2H-pyrazolo[3,4-c]pyridine-2- yl)benzonitrile |
| 19.41 | 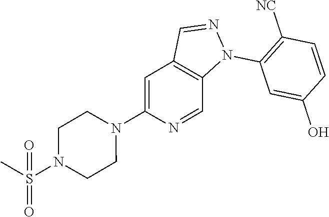 [SMILES: CS(=O)(=O)N1CCN(c2cc3cnn(-c4cc(O)ccc4C#N)c3cn2)CC1] | 4-Hydroxy-2-(5-(4-(methylsulfonyl)piperazin- 1-yl)-1H-pyrazolo[3,4-c]pyridine-1- yl)benzonitrile |
| 19.42 | 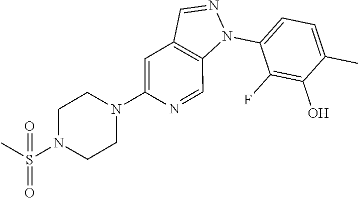 | 2-Fluoro-6-methyl-3-(5-(4- (methylsulfonyl)piperazin-1-yl)-1H- pyrazolo[3,4-c]63yridine-1-yl)phenol |
| 19.43 | 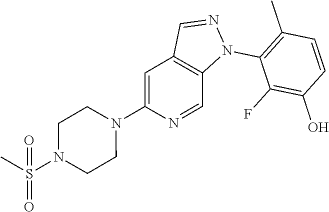 [SMILES: Cc1ccc(O)c(F)c1-n1ncc2cc(N3CCN(S(C)(=O)=O)CC3)ncc21] | 2-Fluoro-4-methyl-3-(5-(4- (methylsulfonyl)piperazin-1-yl)-1H- pyrazolo[3,4-c]pyridine-1-yl)phenol |
| 19.44 | 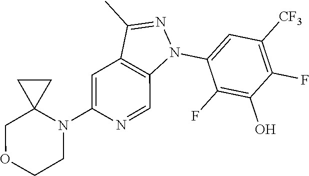 [SMILES: Cc1nn(-c2cc(C(F)(F)F)c(F)c(O)c2F)c2cnc(N3CCOCC34CC4)cc12] | 2,6-Difluoro-3-(3-methyl-5-(7-oxa-4- azaspiro[2.5]octan-4-yl)-1H-pyrazolo[3,4- c]64yridine-1-yl)-5-(trifluoromethyl)phenol |
| 19.45 | 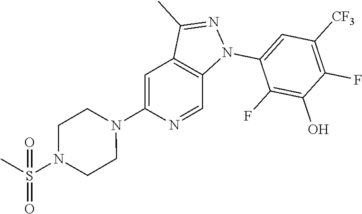 [SMILES: Cc1nn(-c2cc(C(F)(F)F)c(F)c(O)c2F)c2cnc(N3CCN(S(C)(=O)=O)CC3)cc12] | 2,6-Difluoro-3-(3-methyl-5-(4- (methylsulfonyl)piperazin-1-yl)-1H- pyrazolo[3,4-c]pyridine-1-yl)-5- (trifluoromethyl)phenol |
| 19.46 | 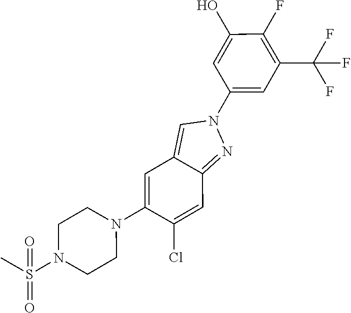 [SMILES: CS(=O)(=O)N1CCN(c2cc3cn(-c4cc(O)c(F)c(C(F)(F)F)c4)nc3cc2Cl)CC1] | 5-(6-Chloro-5-(4-(methylsulfonyl)piperazin-1- yl)-2H-indazol-2-yl)-2-fluoro-3- (trifluoromethyl)phenol |
| 19.47 | 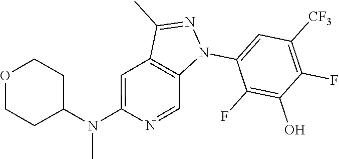 [SMILES: Cc1nn(-c2cc(C(F)(F)F)c(F)c(O)c2F)c2cnc(N(C)C3CCOCC3)cc12] | 2,6-Difluoro-3-(3-methyl-5-(methyl(tetrahydro- 2H-pyran-4-yl)amino)-1H-pyrazolo[3,4- c]pyridin-1-yl)-5-(trifluoromethyl)phenol |
| 19.48 | 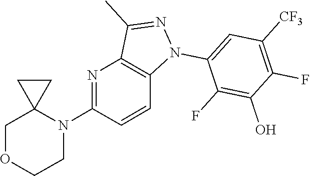 [SMILES: Cc1nn(-c2cc(C(F)(F)F)c(F)c(O)c2F)c2ccc(N3CCOCC34CC4)nc12] | 2,6-Difluoro-3-(3-methyl-5-(7-oxa-4- azaspiro[2.5]octan-4-yl)-1H-pyrazolo[4,3- b]pyridin-1-yl)-5-(trifluoromethyl)phenol |
| 19.49 | 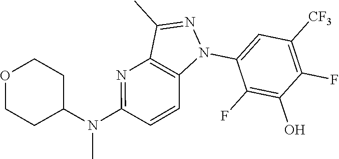 [SMILES: Cc1nn(-c2cc(C(F)(F)F)c(F)c(O)c2F)c2ccc(N(C)C3CCOCC3)nc12] | 2,6-Difluoro-3-(3-methyl-5-(methyl(tetrahydro- 2H-pyran-4-yl)amino)-1H-pyrazolo[4,3- b]pyridin-1-yl)-5-(trifluoromethyl)phenol |
| 19.50 | 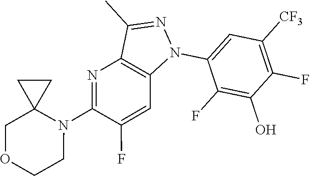 [SMILES: Cc1nn(-c2cc(C(F)(F)F)c(F)c(O)c2F)c2cc(F)c(N3CCOCC34CC4)nc12] | 2,6-Difluoro-3-(6-fluoro-3-methyl-5-(7-oxa-4- azaspiro[2.5]octan-4-yl)-1H-pyrazolo[4,3- b]pyridin-1-yl)-5-(trifluoromethyl)phenol |
| 19.51 | 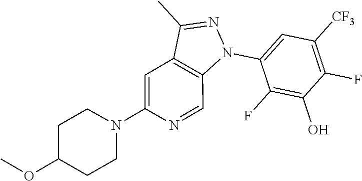 [SMILES: COC1CCN(c2cc3c(C)nn(-c4cc(C(F)(F)F)c(F)c(O)c4F)c3cn2)CC1] | 2,6-Difluoro-3-(5-(4-methoxypiperidin-1-yl)-3- methyl-1H-pyrazolo[3,4-c]64yridine-1-yl)-5- (trifluoromethyl)phenol |
| 19.52 | 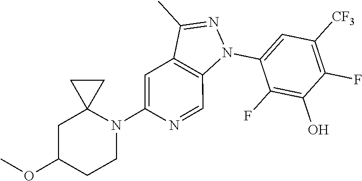 [SMILES: COC1CCN(c2cc3c(C)nn(-c4cc(C(F)(F)F)c(F)c(O)c4F)c3cn2)C2(CC2)C1] | 2,6-Difluoro-3-(5-(7-methoxy-4- azaspiro[2.5]octan-4-yl)-3-methyl-1H- pyrazolo[3,4-c]pyridin-1-yl)-5- (trifluoromethyl)phenol |
| 19.53 | 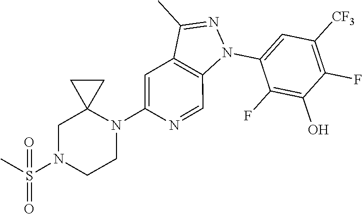 [SMILES: Cc1nn(-c2cc(C(F)(F)F)c(F)c(O)c2F)c2cnc(N3CCN(S(C)(=O)=O)CC34CC4)cc12] | 2,6-Difluoro-3-(3-methyl-5-(7- (methylsulfonyl)-4,7-diazaspiro[2.5]octan-4-yl)- 1H-pyrazolo[3,4-c]pyridin-1-yl)-5- (trifluoromethyl)phenol |
| 19.54 | 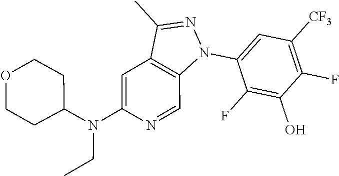 [SMILES: CCN(c1cc2c(C)nn(-c3cc(C(F)(F)F)c(F)c(O)c3F)c2cn1)C1CCOCC1] | 3-(5-(Ethyl(tetrahydro-2H-pyran-4-yl)amino)-3- methyl-1H-pyrazolo[3,4-c]65yridine-1-yl)-2,6- difluoro-5-(trifluoromethyl)phenol |
| 19.55 | 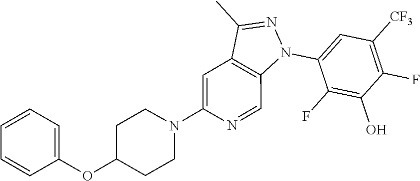 [SMILES: Cc1nn(-c2cc(C(F)(F)F)c(F)c(O)c2F)c2cnc(N3CCC(Oc4ccccc4)CC3)cc12] | 2,6-Difluoro-3-(3-methyl-5-(4- phenoxypiperidin-1-yl)-1H-pyrazolo[3,4- c]65yridine-1-yl)-5-(trifluoromethyl)phenol |
| 19.56 | 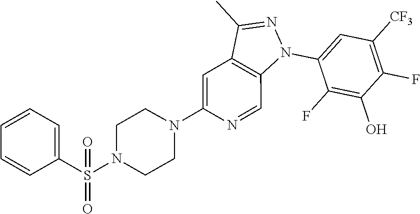 [SMILES: Cc1nn(-c2cc(C(F)(F)F)c(F)c(O)c2F)c2cnc(N3CCN(S(=O)(=O)c4ccccc4)CC3)cc12] | 2,6-Difluoro-3-(3-methyl-5-(4- (phenylsulfonyl)piperazin-1-yl)-1H- pyrazolo[3,4-c]65yridine-1-yl)-5- (trifluoromethyl)phenol |
| 19.57 | 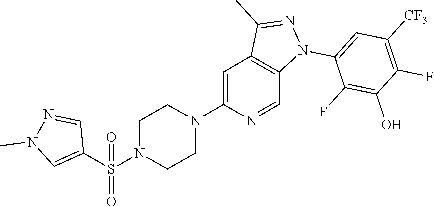 [SMILES: Cc1nn(-c2cc(C(F)(F)F)c(F)c(O)c2F)c2cnc(N3CCN(S(=O)(=O)c4cnn(C)c4)CC3)cc12] | 2,6-Difluoro-3-(3-methyl-5-(4-((1-methyl-1H- pyrazol-4-yl)sulfonyl)piperazin-1-yl)-1H- pyrazolo[3,4-c]pyridin-1-yl)-5- (trifluoromethyl)phenol |
| 19.58 | 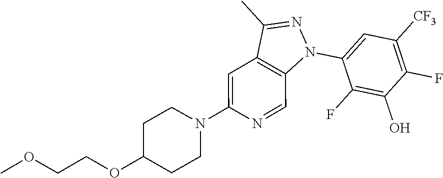 [SMILES: COCCOC1CCN(c2cc3c(C)nn(-c4cc(C(F)(F)F)c(F)c(O)c4F)c3cn2)CC1] | 2,6-Difluoro-3-(5-(4-(2- methoxyethoxy)piperidin-1-yl)-3-methyl-1H- pyrazolo[3,465yridinedin-1-yl)-5- (trifluoromethyl)phenol |
| 19.59 | 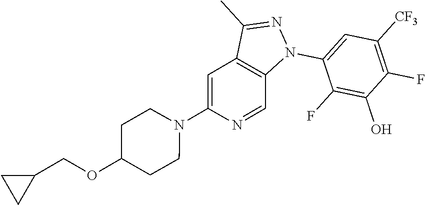 [SMILES: Cc1nn(-c2cc(C(F)(F)F)c(F)c(O)c2F)c2cnc(N3CCC(OCC4CC4)CC3)cc12] | 3-(5-(4-(Cyclopropylmethoxy)piperidin-1-yl)-3- methyl-1H-pyrazolo[3,4-c]65yridine-1-yl)-2,6- difluoro-5-(trifluoromethyl)phenol |
| 19.60 | 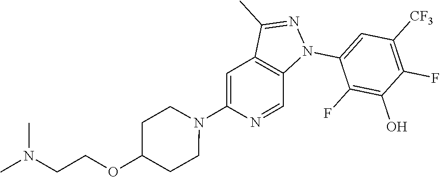 [SMILES: Cc1nn(-c2cc(C(F)(F)F)c(F)c(O)c2F)c2cnc(N3CCC(OCCN(C)C)CC3)cc12] | 3-(5-(4-(2-(Dimethylamino)ethoxy)piperidin-1- yl)-3-methyl-1H-pyrazolo[3,4-c]65yridine-1- yl)-2,6-difluoro-5-(trifluoromethyl)phenol |
| 19.61 | 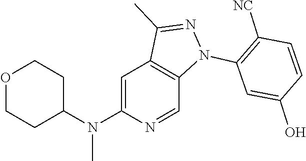 [SMILES: Cc1nn(-c2cc(O)ccc2C#N)c2cnc(N(C)C3CCOCC3)cc12] | 4-Hydroxy-2-(3-methyl-5-(methyl(tetrahydro- 2H-pyran-4-yl)amino)-1H-pyrazolo[3,4- c]pyridin-1-yl)benzonitrile |
| 19.62 | 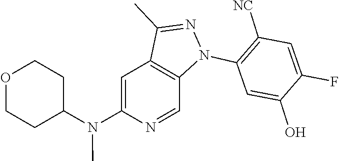 [SMILES: Cc1nn(-c2cc(O)c(F)cc2C#N)c2cnc(N(C)C3CCOCC3)cc12] | 5-Fluoro-4-hydroxy-2-(3-methyl-5- (methyl(tetrahydro-2H-pyran-4-yl)amino)-1H- pyrazolo[3,4-c]pyridin-1-yl)benzonitrile |
| 19.63 | 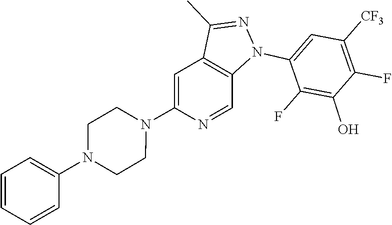 [SMILES: Cc1nn(-c2cc(C(F)(F)F)c(F)c(O)c2F)c2cnc(N3CCN(c4ccccc4)CC3)cc12] | 2,6-Difluoro-3-(3-methyl-5-(4-phenylpiperazin- 1-yl)-1H-pyrazolo[3,4-c]pyridin-1-yl)-5- (trifluoromethyl)phenol |
| 19.64 | 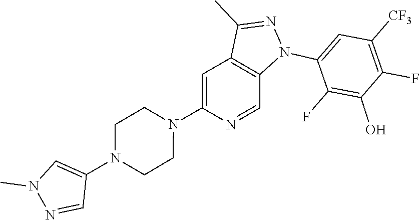 [SMILES: Cc1nn(-c2cc(C(F)(F)F)c(F)c(O)c2F)c2cnc(N3CCN(c4cnn(C)c4)CC3)cc12] | 2,6-Difluoro-3-(3-methyl-5-(4-(1-methyl-1H- pyrazol-4-yl)piperazin-1-yl)-1H-pyrazolo[3,4- c]pyridin-1-yl)-5-(trifluoromethyl)phenol |
| 19.65 | 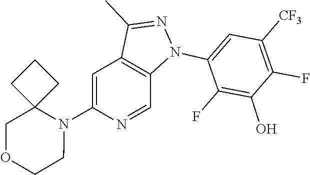 [SMILES: Cc1nn(-c2cc(C(F)(F)F)c(F)c(O)c2F)c2cnc(N3CCOCC34CCC4)cc12] | 2,6-Difluoro-3-(3-methyl-5-(8-oxa-5- azaspiro[3.5]nonan-5-yl)-1H-pyrazolo[3,4- c]pyridin-1-yl)-5-(trifluoromethyl)phenol |
| 19.66 | 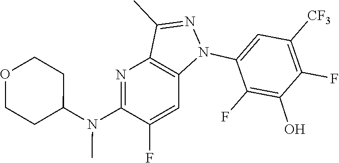 [SMILES: Cc1nn(-c2cc(C(F)(F)F)c(F)c(O)c2F)c2cc(F)c(N(C)C3CCOCC3)nc12] | 2,6-Difluoro-3-(6-fluoro-3-methyl-5- (methyl(tetrahydro-2H-pyran-4-yl)amino)-1H- pyrazolo[4,3-b]pyridin-1-yl)-5- (trifluoromethyl)phenol |
| 19.67 | 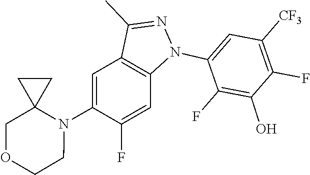 [SMILES: Cc1nn(-c2cc(C(F)(F)F)c(F)c(O)c2F)c2cc(F)c(N3CCOCC34CC4)cc12] | 2,6-Difluoro-3-(6-fluoro-3-methyl-5-(7-oxa-4- azaspiro[2.5]octan-4-yl)-1H-indazol-1-yl)-5- (trifluoromethyl)phenol |
| 19.68 | 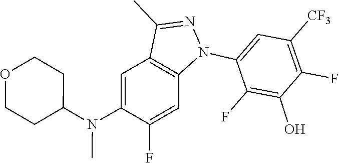 [SMILES: Cc1nn(-c2cc(C(F)(F)F)c(F)c(O)c2F)c2cc(F)c(N(C)C3CCOCC3)cc12] | 2,6-Difluoro-3-(6-fluoro-3-methyl-5- (methyl(tetrahydro-2H-pyran-4-yl)amino)-1H- indazol-1-yl)-5-(trifluoromethyl)phenol |
| 19.69 | 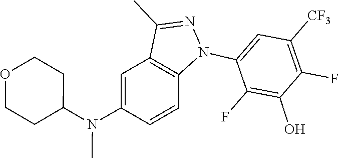 [SMILES: Cc1nn(-c2cc(C(F)(F)F)c(F)c(O)c2F)c2ccc(N(C)C3CCOCC3)cc12] | 2,6-Difluoro-3-(3-methyl-5-(methyl(tetrahydro- 2H-pyran-4-yl)amino)-1H-indazol-1-yl)-5- (trifluoromethyl)phenol |
| 19.70 | 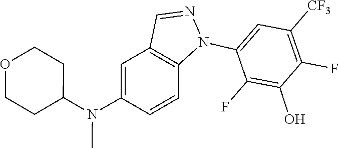 [SMILES: CN(c1ccc2c(cnn2-c2cc(C(F)(F)F)c(F)c(O)c2F)c1)C1CCOCC1] | 2,6-Difluoro-3-(5-(methyl(tetrahydro-2H-pyran- 4-yl)amino)-1H-indazol-1-yl)-5- (trifluoromethyl)phenol |
| 19.71 | 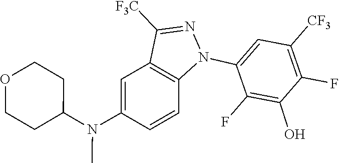 [SMILES: CN(c1ccc2c(c1)c(C(F)(F)F)nn2-c1cc(C(F)(F)F)c(F)c(O)c1F)C1CCOCC1] | 2,6-Difluoro-3-(5-(methyl(tetrahydro-2H-pyran- 4-yl)amino)-3-(trifluoromethyl)-1H-indazol-1- yl)-5-(trifluoromethyl)phenol |
| 19.72 | 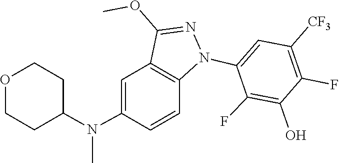 [SMILES: COc1nn(-c2cc(C(F)(F)F)c(F)c(O)c2F)c2ccc(N(C)C3CCOCC3)cc12] | 2,6-Difluoro-3-(3-methoxy-5- (methyl(tetrahydro-2H-pyran-4-yl)amino)-1H- indazol-1-yl)-5-(trifluoromethyl)phenol |
| 19.73 | 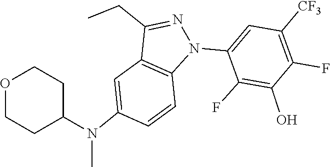 [SMILES: CCc1nn(-c2cc(C(F)(F)F)c(F)c(O)c2F)c2ccc(N(C)C3CCOCC3)cc12] | 3-(3-Ethyl-5-(methyl(tetrahydro-2H-pyran-4- yl)amino)-1H-indazol-1-yl)-2,6-difluoro-5- (trifluoromethyl)phenol |
| 19.74 | 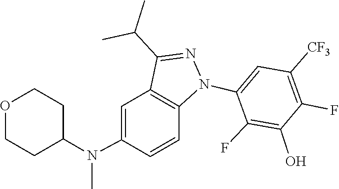 [SMILES: CC(C)c1nn(-c2cc(C(F)(F)F)c(F)c(O)c2F)c2ccc(N(C)C3CCOCC3)cc12] | 2,6-Difluoro-3-(3-isopropyl-5- (methyl(tetrahydro-2H-pyran-4-yl)amino)-1H- indazol-1-yl)-5-(trifluoromethyl)phenol |
| 19.75 | 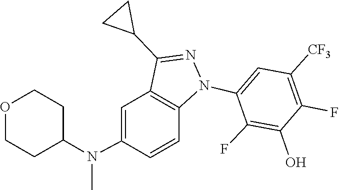 [SMILES: CN(c1ccc2c(c1)c(C1CC1)nn2-c1cc(C(F)(F)F)c(F)c(O)c1F)C1CCOCC1] | 3-(3-Cyclopropyl-5-(methyl(tetrahydro-2H- pyran-4-yl)amino)-1H-indazol-1-yl)-2,6- difluoro-5-(trifluoromethyl)phenol |
| 19.76 | 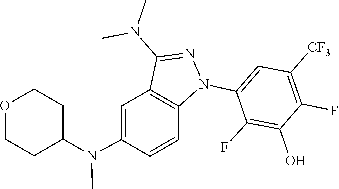 [SMILES: CN(C)c1nn(-c2cc(C(F)(F)F)c(F)c(O)c2F)c2ccc(N(C)C3CCOCC3)cc12] | 3-(3-(Dimethylamino)-5-(methyl(tetrahydro- 2H-pyran-4-yl)amino)-1H-indazol-1-yl)-2,6- difluoro-5-(trifluoromethyl)phenol |
| 19.77 | 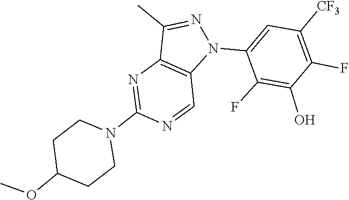 [SMILES: COC1CCN(c2ncc3c(n2)c(C)nn3-c2cc(C(F)(F)F)c(F)c(O)c2F)CC1] | 2,6-Difluoro-3-(5-(4-methoxypiperidin-1-yl)-3- methyl-1H-pyrazolo[4,3-d]pyrimidin-1-yl)-5- (trifluoromethyl)phenol |
| 19.78 | 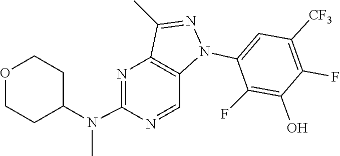 [SMILES: Cc1nn(-c2cc(C(F)(F)F)c(F)c(O)c2F)c2cnc(N(C)C3CCOCC3)nc12] | 2,6-Difluoro-3-(3-methyl-5-(methyl(tetrahydro- 2H-pyran-4-yl)amino)-1H-pyrazolo[4,3- d]pyrimidin-1-yl)-5-(trifluoromethyl)phenol |
| 19.79 | 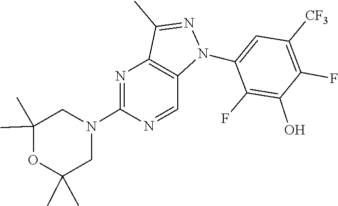 [SMILES: Cc1nn(-c2cc(C(F)(F)F)c(F)c(O)c2F)c2cnc(N3CC(C)(C)OC(C)(C)C3)nc12] | 2,6-Difluoro-3-(3-methyl-5-(2,2,6,6- tetramethylmorpholino)-1H-pyrazolo[4,3- d]pyrimidin-1-yl)-5-(trifluoromethyl)phenol |
| 19.80 | 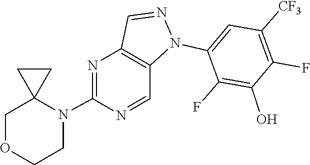 [SMILES: Oc1c(F)c(-n2ncc3nc(N4CCOCC45CC5)ncc32)cc(C(F)(F)F)c1F] | 3-(5-(7-Oxa-4-azaspiro[2.5]octan-4-yl)-1H- pyrazolo[4,3-d]pyrimidin-1-yl)-2,6-difluoro-5- (trifluoromethyl)phenol |
| 19.81 | 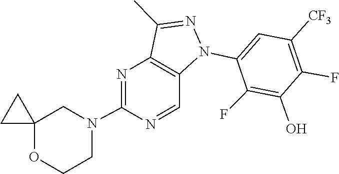 [SMILES: Cc1nn(-c2cc(C(F)(F)F)c(F)c(O)c2F)c2cnc(N3CCOC4(CC4)C3)nc12] | 2,6-Difluoro-3-(3-methyl-5-(4-oxa-7- azaspiro[2.5]octan-7-yl)-1H-pyrazolo[4,3- d]pyrimidin-1-yl)-5-(trifluoromethyl)phenol |
| 19.82 | 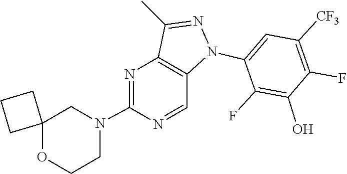 [SMILES: Cc1nn(-c2cc(C(F)(F)F)c(F)c(O)c2F)c2cnc(N3CCOC4(CCC4)C3)nc12] | 2,6-Difluoro-3-(3-methyl-5-(5-oxa-8- azaspiro[3.5]nonan-8-yl)-1H-pyrazolo[4,3- d]pyrimidin-1-yl)-5-(trifluoromethyl)phenol |
| 19.83 | 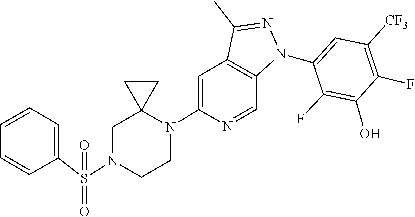 [SMILES: Cc1nn(-c2cc(C(F)(F)F)c(F)c(O)c2F)c2cnc(N3CCN(S(=O)(=O)c4ccccc4)CC34CC4)cc12] | 2,6-Difluoro-3-(3-methyl-5-(7- (phenylsulfonyl)-4,7-diazaspiro[2.5]octan-4-yl)- 1H-pyrazolo[3,4-c]pyridin-1-yl)-5- (trifluoromethyl)phenol |
| 19.84 | 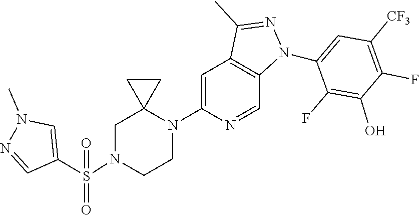 [SMILES: Cc1nn(-c2cc(C(F)(F)F)c(F)c(O)c2F)c2cnc(N3CCN(S(=O)(=O)c4cnn(C)c4)CC34CC4)cc12] | 2,6-Difluoro-3-(3-methyl-5-(7-((1-methyl-1H- pyrazol-4-yl)sulfonyl)-4,7-diazaspiro[2.5]octan- 4-yl)-1H-pyrazolo[3,4-c]pyridin-1-yl)-5- (trifluoromethyl)phenol |
| 19.85 | 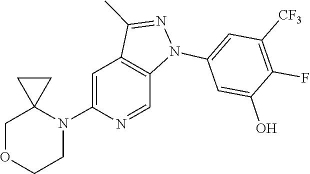 [SMILES: Cc1nn(-c2cc(O)c(F)c(C(F)(F)F)c2)c2cnc(N3CCOCC34CC4)cc12] | 2-Fluoro-5-(3-methyl-5-(7-oxa-4- azaspiro[2.5]octan-4-yl)-1H-pyrazolo[3,4- c]pyridin-1-yl)-3-(trifluoromethyl)phenol |
| 19.86 | 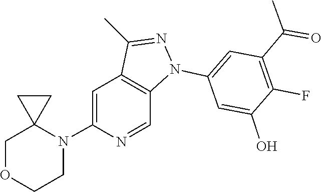 [SMILES: CC(=O)c1cc(-n2nc(C)c3cc(N4CCOCC45CC5)ncc32)cc(O)c1F] | 1-(2-Fluoro-3-hydroxy-5-(3-methyl-5-(7-oxa-4- azaspiro[2.5]octan-4-yl)-1H-pyrazolo[3,4- c]pyridin-1-yl)phenyl)ethanone |
| 19.87 | 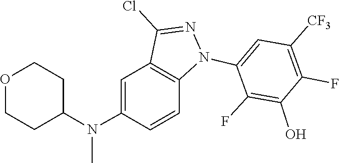 [SMILES: CN(c1ccc2c(c1)c(Cl)nn2-c1cc(C(F)(F)F)c(F)c(O)c1F)C1CCOCC1] | 3-(3-Chloro-5-(methyl(tetrahydro-2H-pyran-4- yl)amino)-1H-indazol-1-yl)-2,6-difluoro-5- (trifluoromethyl)phenol |
| 19.88 | 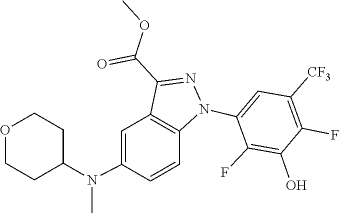 [SMILES: COC(=O)c1nn(-c2cc(C(F)(F)F)c(F)c(O)c2F)c2ccc(N(C)C3CCOCC3)cc12] | Methyl 1-(2,4-difluoro-3-hydroxy-5- (trifluoromethyl)phenyl)-5-(methyl(tetrahydro- 2H-pyran-4-yl)amino)-1H-indazole-3- carboxylate |
| 19.89 | 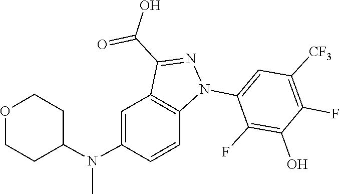 [SMILES: CN(c1ccc2c(c1)c(C(=O)O)nn2-c1cc(C(F)(F)F)c(F)c(O)c1F)C1CCOCC1] | 1-(2,4-Difluoro-3-hydroxy-5- (trifluoromethyl)phenyl)-5-(methyl(tetrahydro- 2H-pyran-4-yl)amino)-1H-indazole-3- carboxylic Acid |
| 19.90 | 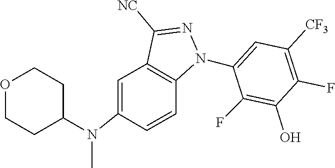 [SMILES: CN(c1ccc2c(c1)c(C#N)nn2-c1cc(C(F)(F)F)c(F)c(O)c1F)C1CCOCC1] | 1-(2,4-Difluoro-3-hydroxy-5- (trifluoromethyl)phenyl)-5-(methyl(tetrahydro- 2H-pyran-4-yl)amino)-1H-indazole-3- carbonitrile |
| 19.91 | 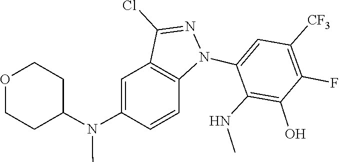 [SMILES: CNc1c(-n2nc(Cl)c3cc(N(C)C4CCOCC4)ccc32)cc(C(F)(F)F)c(F)c1O] | 3-(3-Chloro-5-(methyl(tetrahydro-2H-pyran-4- yl)amino)-1H-indazol-1-yl)-6-fluoro-2- (methylamino)-5-(trifluoromethyl)phenol |
| 19.92 | 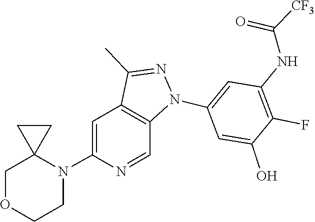 [SMILES: Cc1nn(-c2cc(O)c(F)c(NC(=O)C(F)(F)F)c2)c2cnc(N3CCOCC34CC4)cc12] | 2,2,2-Trifluoro-N-(2-fluoro-3-hydroxy-5-(3- methyl-5-(7-oxa-4-azaspiro[2.5]octan-4-yl)-1H- pyrazolo[3,4-c]pyridin-1-yl)phenyl)acetamide |
| 19.93 | 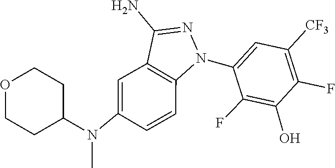 [SMILES: CN(c1ccc2c(c1)c(N)nn2-c1cc(C(F)(F)F)c(F)c(O)c1F)C1CCOCC1] | 3-(3-Amino-5-(methyl(tetrahydro-2H-pyran-4- yl)amino)-1H-indazol-1-yl)-2,6-difluoro-5- (trifluoromethyl)phenol |
| 19.94 | 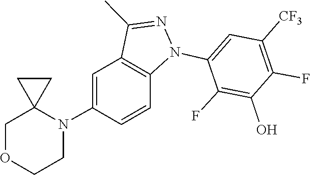 [SMILES: Cc1nn(-c2cc(C(F)(F)F)c(F)c(O)c2F)c2ccc(N3CCOCC34CC4)cc12] | 2,6-Difluoro-3-(3-methyl-5-(7-oxa-4- azaspiro[2.5]octan-4-yl)-1H-indazol-1-yl)-5- (trifluoromethyl)phenol |
| 19.95 | 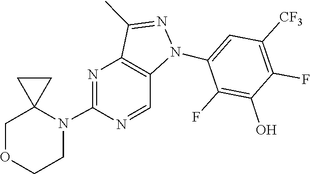 [SMILES: Cc1nn(-c2cc(C(F)(F)F)c(F)c(O)c2F)c2cnc(N3CCOCC34CC4)nc12] | 2,6-Difluoro-3-(3-methyl-5-(7-oxa-4- azaspiro[2.5]octan-4-yl)-1H-pyrazolo[4,3- d]pyrimidin-1-yl)-5-(trifluoromethyl)phenol |
| 19.96 | 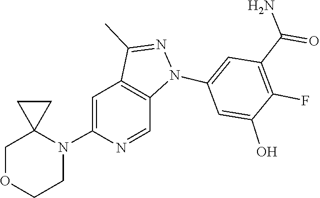 [SMILES: Cc1nn(-c2cc(O)c(F)c(C(N)=O)c2)c2cnc(N3CCOCC34CC4)cc12] | 2-Fluoro-3-hydroxy-5-(3-methyl-5-(7-oxa-4- azaspiro[2.5]octan-4-yl)-1H-pyrazolo[3,4- c]pyridin-1-yl)benzamide |
| 19.97 | 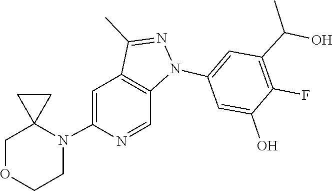 [SMILES: Cc1nn(-c2cc(O)c(F)c(C(C)O)c2)c2cnc(N3CCOCC34CC4)cc12] | 2-Fluoro-3-(1-hydroxyethyl)-5-(3-methyl-5-(7- oxa-4-azaspiro[2.5]octan-4-yl)-1H- pyrazolo[3,4-c]pyridin-1-yl)phenol |
| 19.98 | 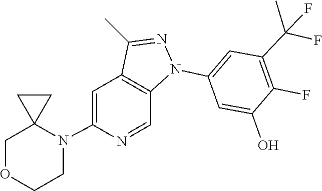 [SMILES: Cc1nn(-c2cc(O)c(F)c(C(C)(F)F)c2)c2cnc(N3CCOCC34CC4)cc12] | 3-(1,1-Difluoroethyl)-2-fluoro-5-(3-methyl-5- (7-oxa-4-azaspiro[2.5]octan-4-yl)-1H- pyrazolo[3,4-c]pyridin-1-yl)phenol |
| 19.99 | 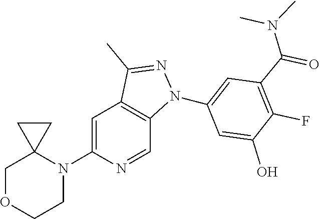 [SMILES: Cc1nn(-c2cc(O)c(F)c(C(=O)N(C)C)c2)c2cnc(N3CCOCC34CC4)cc12] | 2-Fluoro-3-hydroxy-N,N-dimethyl-5-(3-methyl- 5-(7-oxa-4-azaspiro[2.5]octan-4-yl)-1H- pyrazolo[3,4-c]pyridin-1-yl)benzamide |
| 19.100 | 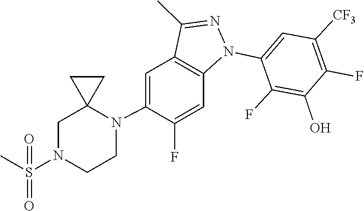 [SMILES: Cc1nn(-c2cc(C(F)(F)F)c(F)c(O)c2F)c2cc(F)c(N3CCN(S(C)(=O)=O)CC34CC4)cc12] | 2,6-Difluoro-3-(6-fluoro-3-methyl-5-(7- (methylsulfonyl)-4,7-diazaspiro[2.5]octan-4-yl)- 1H-indazol-1-yl)-5-(trifluoromethyl)phenol |
| 19.101 | 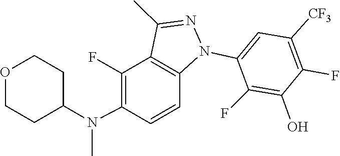 [SMILES: Cc1nn(-c2cc(C(F)(F)F)c(F)c(O)c2F)c2ccc(N(C)C3CCOCC3)c(F)c12] | 2,6-Difluoro-3-(4-fluoro-3-methyl-5- (methyl(tetrahydro-2H-pyran-4-yl)amino)-1H- indazol-1-yl)-5-(trifluoromethyl)phenol |
| 19.102 | 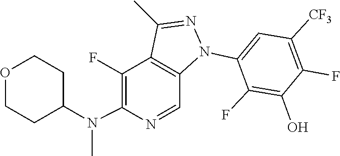 [SMILES: Cc1nn(-c2cc(C(F)(F)F)c(F)c(O)c2F)c2cnc(N(C)C3CCOCC3)c(F)c12] | 2,6-Difluoro-3-(4-fluoro-3-methyl-5- (methyl(tetrahydro-2H-pyran-4-yl)amino)-1H- pyrazolo[3,4-c]pyridin-1-yl)-5- (trifluoromethyl)phenol |
| 19.103 | 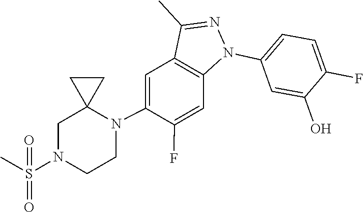 [SMILES: Cc1nn(-c2ccc(F)c(O)c2)c2cc(F)c(N3CCN(S(C)(=O)=O)CC34CC4)cc12] | 2-Fluoro-5-(6-fluoro-3-methyl-5-(7- (methylsulfonyl)-4,7-diazaspiro[2.5]octan-4-yl)- 1H-indazol-1-yl)phenol |
| 19.104 | 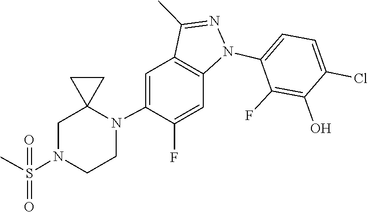 [SMILES: Cc1nn(-c2ccc(Cl)c(O)c2F)c2cc(F)c(N3CCN(S(C)(=O)=O)CC34CC4)cc12] | 6-Chloro-2-fluoro-3-(6-fluoro-3-methyl-5-(7- (methylsulfonyl)-4,7-diazaspiro[2.5]octan-4-yl)- 1H-indazol-1-yl)phenol |
| 19.105 | 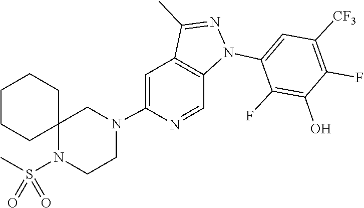 [SMILES: Cc1nn(-c2cc(C(F)(F)F)c(F)c(O)c2F)c2cnc(N3CCN(S(C)(=O)=O)C4(CCCCC4)C3)cc12] | 2,6-Difluoro-3-(3-methyl-5-(1- (methylsulfonyl)-1,4-diazaspiro[5.5]undecan-4- yl)-1H-pyrazolo[3,4-c]pyridin-1-yl)-5- (trifluoromethyl)phenol |
| 19.106 | 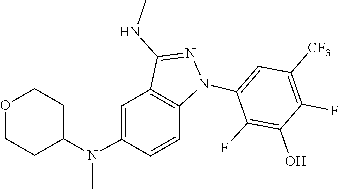 [SMILES: CNc1nn(-c2cc(C(F)(F)F)c(F)c(O)c2F)c2ccc(N(C)C3CCOCC3)cc12] | 2,6-Difluoro-3-(5-(methyl(tetrahydro-2H-pyran- 4-yl)amino)-3-(methylamino)-1H-indazol-1-yl)- 5-(trifluoromethyl)phenol |
| 19.107 | 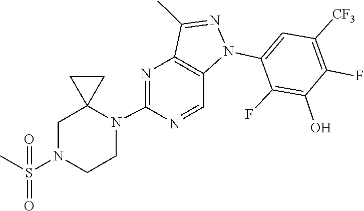 [SMILES: Cc1nn(-c2cc(C(F)(F)F)c(F)c(O)c2F)c2cnc(N3CCN(S(C)(=O)=O)CC34CC4)nc12] | 2,6-Difluoro-3-(3-methyl-5-(7- (methylsulfonyl)-4,7-diazaspiro[2.5]octan-4-yl) 1H-pyrazolo[4,3-d]pyrimidin-1-yl)-5- (trifluoromethyl)phenol |
| 19.108 | 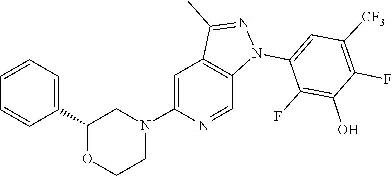 [SMILES: Cc1nn(-c2cc(C(F)(F)F)c(F)c(O)c2F)c2cnc(N3CCO[C@H](c4ccccc4)C3)cc12] | (R)-2,6-Difluoro-3-(3-methyl-5-(2- phenylmorpholino)-1H-pyrazolo[3,4-c]pyridin- 1-yl)-5-(trifluoromethyl)phenol |
| 19.109 | 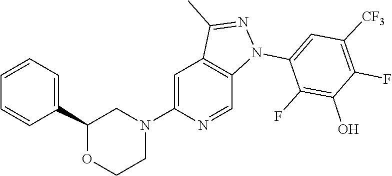 [SMILES: Cc1nn(-c2cc(C(F)(F)F)c(F)c(O)c2F)c2cnc(N3CCO[C@@H](c4ccccc4)C3)cc12] | (S)-2,6-Difluoro-3-(3-methyl-5-(2- phenylmorpholino)-1H-pyrazolo[3,4-c]pyridin- 1-yl)-5-(trifluoromethyl)phenol |
| 19.110 | 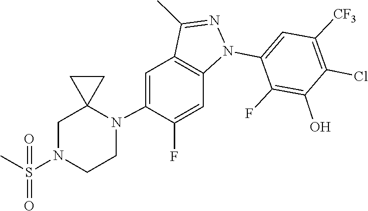 [SMILES: Cc1nn(-c2cc(C(F)(F)F)c(Cl)c(O)c2F)c2cc(F)c(N3CCN(S(C)(=O)=O)CC34CC4)cc12] | 2-Chloro-6-fluoro-5-(6-fluoro-3-methyl-5-(7- (methylsulfonyl)-4,7-diazaspiro[2.5]octan-4-yl)- 1H-indazol-1-yl)-3-(trifluoromethyl)phenol |
| 19.111 | 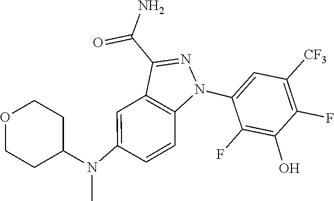 [SMILES: CN(c1ccc2c(c1)c(C(N)=O)nn2-c1cc(C(F)(F)F)c(F)c(O)c1F)C1CCOCC1] | 1-(2,4-Difluoro-3-hydroxy-5- (trifluoromethyl)phenyl)-5-(methyl(tetrahydro- 2H-pyran-4-yl)amino)-1H-indazole-3- carboxamide |
| 19.112 | 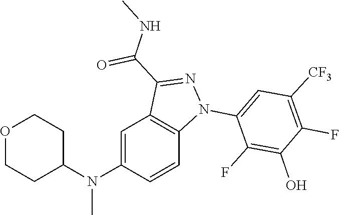 [SMILES: CNC(=O)c1nn(-c2cc(C(F)(F)F)c(F)c(O)c2F)c2ccc(N(C)C3CCOCC3)cc12] | 1-(2,4-Difluoro-3-hydroxy-5- (trifluoromethyl)phenyl)-N-methyl-5- (methyl(tetrahydro-2H-pyran-4-yl)amino)-1H- indazole-3-carboxamide |
| 19.113 | 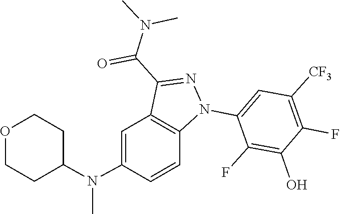 [SMILES: CN(C)C(=O)c1nn(-c2cc(C(F)(F)F)c(F)c(O)c2F)c2ccc(N(C)C3CCOCC3)cc12] | 1-(2,4-Difluoro-3-hydroxy-5- (trifluoromethyl)phenyl)-N,N-dimethyl-5- (methyl(tetrahydro-2H-pyran-4-yl)amino)-1H- indazole-3-carboxamide |
| 19.114 | 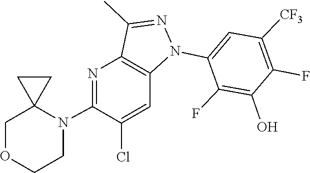 [SMILES: Cc1nn(-c2cc(C(F)(F)F)c(F)c(O)c2F)c2cc(Cl)c(N3CCOCC34CC4)nc12] | 3-(6-Chloro-3-methyl-5-(7-oxa-4- azaspiro[2.5]octan-4-yl)-1H-pyrazolo[4,3- b]pyridin-1-yl)-2,6-difluoro-5- (trifluoromethyl)phenol |
| 19.115 | 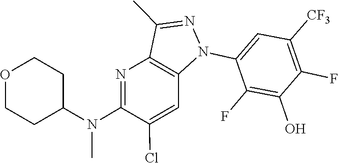 [SMILES: Cc1nn(-c2cc(C(F)(F)F)c(F)c(O)c2F)c2cc(Cl)c(N(C)C3CCOCC3)nc12] | 3-(6-Chloro-3-methyl-5-(methyl(tetrahydro-2H- pyran-4-yl)amino)-1H-pyrazolo[4,3-b]pyridin- 1-yl)-2,6-difluoro-5-(trifluoromethyl)phenol |
| 19.116 | 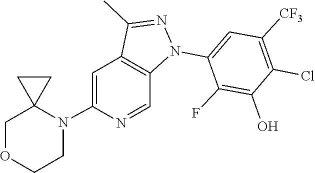 [SMILES: Cc1nn(-c2cc(C(F)(F)F)c(Cl)c(O)c2F)c2cnc(N3CCOCC34CC4)cc12] | 2-Chloro-6-fluoro-5-(3-methyl-5-(7-oxa-4- azaspiro[2.5]octan-4-yl)-1H-pyrazolo[3,4- c]pyridin-1-yl)-3-(trifluoromethyl)phenol |
| 19.117 | 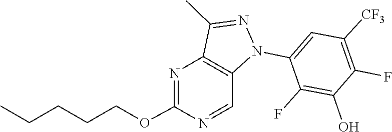 [SMILES: CCCCCOc1ncc2c(n1)c(C)nn2-c1cc(C(F)(F)F)c(F)c(O)c1F] | 2,6-Difluoro-3-(3-methyl-5-(pentyloxy)-1H- pyrazolo[4,3-d]pyrimidin-1-yl)-5- (trifluoromethyl)phenol |
| 19.118 | 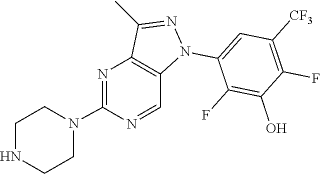 [SMILES: Cc1nn(-c2cc(C(F)(F)F)c(F)c(O)c2F)c2cnc(N3CCNCC3)nc12] | 2,6-Difluoro-3-(3-methyl-5-(piperazin-1-yl)- 1H-pyrazolo[4,3-d]pyrimidin-1-yl)-5- (trifluoromethyl)phenol |
| 19.119 | 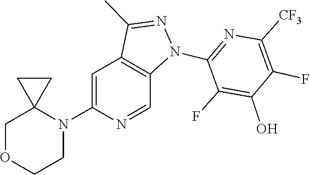 [SMILES: Cc1nn(-c2nc(C(F)(F)F)c(F)c(O)c2F)c2cnc(N3CCOCC34CC4)cc12] | 3,5-Difluoro-2-(3-methyl-5-(7-oxa-4- azaspiro[2.5]octan-4-yl)-1H-pyrazolo[3,4- c]pyridin-1-yl)-6-(trifluoromethyl)pyridin-4-ol |
| 19.120 | 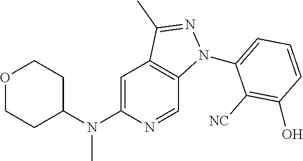 [SMILES: Cc1nn(-c2cccc(O)c2C#N)c2cnc(N(C)C3CCOCC3)cc12] | 2-Hydroxy-6-(3-methyl-5-(methyl(tetrahydro- 2H-pyran-4-yl)amino)-1H-pyrazolo[3,4- c]pyridin-1-yl)benzonitrile |
| 19.121 | 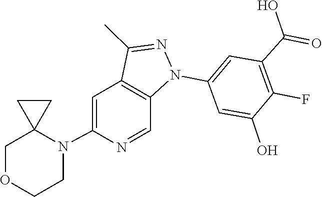 [SMILES: Cc1nn(-c2cc(O)c(F)c(C(=O)O)c2)c2cnc(N3CCOCC34CC4)cc12] | 2-Fluoro-3-hydroxy-5-(3-methyl-5-(7-oxa-4- azaspiro[2.5]octan-4-yl)-1H-pyrazolo[3,4- c]pyridin-1-yl)benzoic Acid |
| 19.122 | 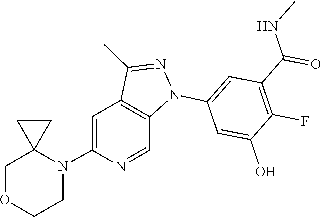 [SMILES: CNC(=O)c1cc(-n2nc(C)c3cc(N4CCOCC45CC5)ncc32)cc(O)c1F] | 2-Fluoro-3-hydroxy-N-methyl-5-(3-methyl-5- (7-oxa-4-azaspiro[2.5]octan-4-yl)-1H- pyrazolo[3,4-c]pyridin-1-yl)benzamide |
| 19.123 | 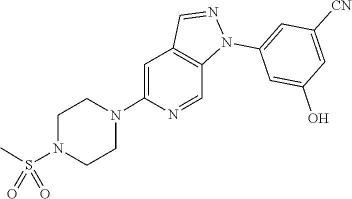 [SMILES: CS(=O)(=O)N1CCN(c2cc3cnn(-c4cc(O)cc(C#N)c4)c3cn2)CC1] | 3-Hydroxy-5-(5-(4-(methylsulfony])piperazin- 1-yl)-1H-pyrazolo[3,4-c]pyridin-1- yl)benzonitrile |
| 20 | 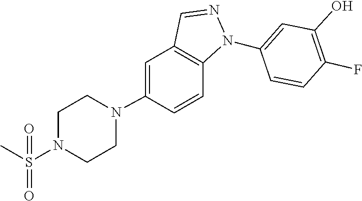 [SMILES: CS(=O)(=O)N1CCN(c2ccc3c(cnn3-c3ccc(F)c(O)c3)c2)CC1] | 2-Fluoro-5-(5-(4-(methylsulfonyl)piperazin-1- yl)-1H-indazol-1-yl)phenol |
| 20.01 | 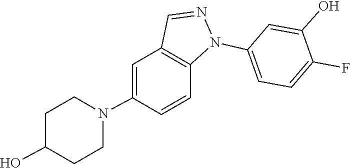 [SMILES: Oc1cc(-n2ncc3cc(N4CCC(O)CC4)ccc32)ccc1F] | 1-(1-(4-Fluoro-3-hydroxyphenyl)-1H-indazol-5- yl)piperidin-4-ol |
| 20.02 | 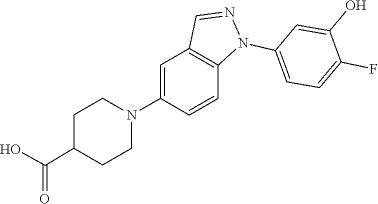 [SMILES: O=C(O)C1CCN(c2ccc3c(cnn3-c3ccc(F)c(O)c3)c2)CC1] | 1-(1-(4-Fluoro-3-hydroxyphenyl)-1H-indazol-5- yl)piperidine-4-carboxylic acid |
| 20.03 | 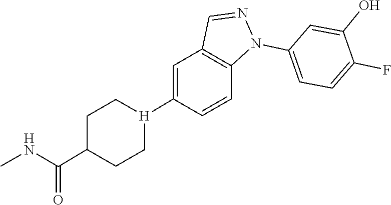 | 1-(1-(4-Fluoro-3-hydroxyphenyl)-1H-indazol-5- yl)-N-methylpiperidine-4-carboxamide |
| 20.04 | 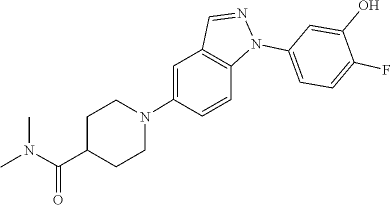 [SMILES: CN(C)C(=O)C1CCN(c2ccc3c(cnn3-c3ccc(F)c(O)c3)c2)CC1] | 1-(1-(4-Fluoro-3-hydroxyphenyl)-1H-indazol-5- yl)-N,N-dimethylpiperidine-4-carboxamide |
| 20.05 | 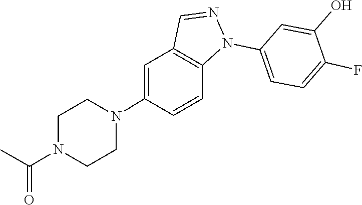 [SMILES: CC(=O)N1CCN(c2ccc3c(cnn3-c3ccc(F)c(O)c3)c2)CC1] | 1-(4-(1-(4-Fluoro-3-hydroxyphenyl)-1H- indazol-5-yl)piperazin-1-yl)ethan-1-one |
| 21 | 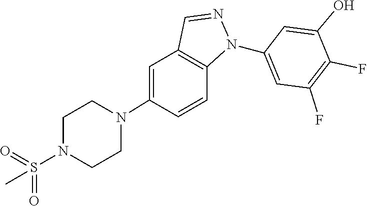 [SMILES: CS(=O)(=O)N1CCN(c2ccc3c(cnn3-c3cc(O)c(F)c(F)c3)c2)CC1] | 2,3-Difluoro-5-(5-(4-(methylsulfonyl)piperazin- 1-yl)-1H-indazol-1-yl)phenol |
| 21.01 | 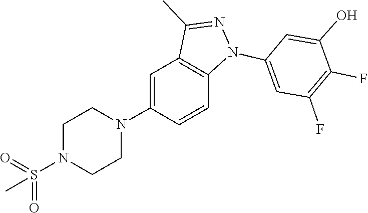 [SMILES: Cc1nn(-c2cc(O)c(F)c(F)c2)c2ccc(N3CCN(S(C)(=O)=O)CC3)cc12] | 2,3-Difluoro-5-(3-methyl-5-(4- (methylsulfonyl)piperazin-1-yl)-1H-indazol-1- yl)phenol |
| 21.02 | 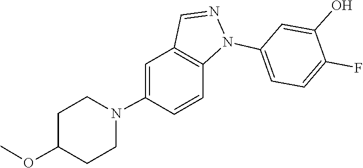 [SMILES: COC1CCN(c2ccc3c(cnn3-c3ccc(F)c(O)c3)c2)CC1] | 2-Fluoro-5-(5-(4-methoxypiperidin-1-yl)-1H- indazol-1-yl)phenol |
| 21.03 | 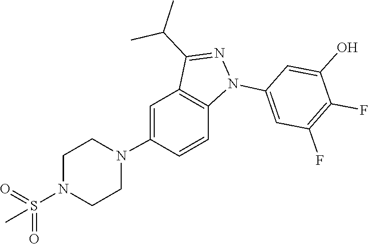 [SMILES: CC(C)c1nn(-c2cc(O)c(F)c(F)c2)c2ccc(N3CCN(S(C)(=O)=O)CC3)cc12] | 2,3-Difluoro-5-(3-isopropyl-5-(4- (methylsulfonyl)piperazin-1-yl)-1H-indazol-1- yl)phenol |
| 21.04 | 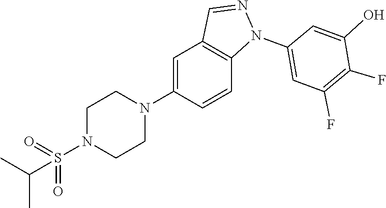 [SMILES: CC(C)S(=O)(=O)N1CCN(c2ccc3c(cnn3-c3cc(O)c(F)c(F)c3)c2)CC1] | 2,3-Difluoro-5-(5-(4- (isopropylsulfonyl)piperazin-1-yl)-1H-indazol- 1-yl)phenol |
| 21.05 | 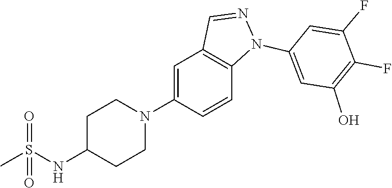 [SMILES: CS(=O)(=O)NC1CCN(c2ccc3c(cnn3-c3cc(O)c(F)c(F)c3)c2)CC1] | N-(1-(1-(3,4-Difluoro-5-hydroxyphenyl)-1H- indazol-5-yl)piperidin-4-yl)methanesulfonamide |
| 21.06 | 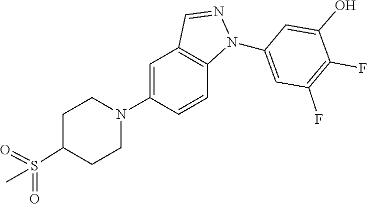 [SMILES: CS(=O)(=O)C1CCN(c2ccc3c(cnn3-c3cc(O)c(F)c(F)c3)c2)CC1] | 2,3-Difluoro-5-(5-(4-(methylsulfonyl)piperidin- 1-yl)-1H-indazol-1-yl)phenol |
| 21.07 | 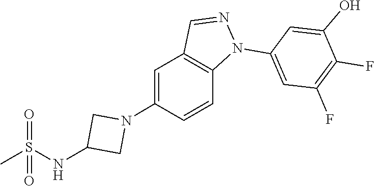 [SMILES: CS(=O)(=O)NC1CN(c2ccc3c(cnn3-c3cc(O)c(F)c(F)c3)c2)C1] | N-(1-(1-(3,4-Difluoro-5-hydroxyphenyl)-1H- indazol-5-yl)azetidine-3-yl)methanesulfonamide |
| 21.08 | 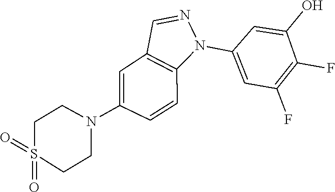 [SMILES: O=S1(=O)CCN(c2ccc3c(cnn3-c3cc(O)c(F)c(F)c3)c2)CC1] | 4-(1-(3,4-Difluoro-5-hydroxyphenyl)-1H- indazol-5-yl)thiomorpholine 1,1-dioxide |
| 21.09 | 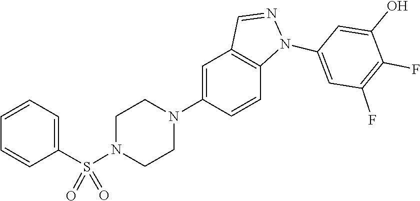 [SMILES: O=S(=O)(c1ccccc1)N1CCN(c2ccc3c(cnn3-c3cc(O)c(F)c(F)c3)c2)CC1] | 2,3-Difluoro-5-(5-(4-(phenylsulfonyl)piperazin- 1-yl)-1H-indazol-1-yl)phenol |
| 21.10 | 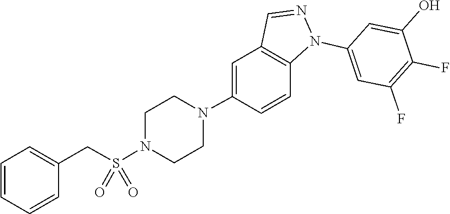 [SMILES: O=S(=O)(Cc1ccccc1)N1CCN(c2ccc3c(cnn3-c3cc(O)c(F)c(F)c3)c2)CC1] | 5-(5-(4-(Benzylsulfonyl)piperazin-1-yl)-1H- indazol-1-yl)-2,3-difluorophenol |
| 21.11 | 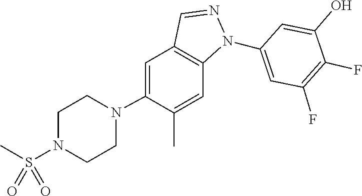 [SMILES: Cc1cc2c(cnn2-c2cc(O)c(F)c(F)c2)cc1N1CCN(S(C)(=O)=O)CC1] | 2,3-Difluoro-5-(6-methyl-5-(4- (methylsulfonyl)piperazin-1-yl)-1H-indazol-1- yl)phenol |
| 21.12 | 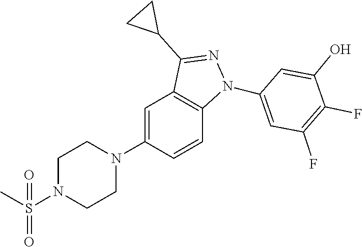 [SMILES: CS(=O)(=O)N1CCN(c2ccc3c(c2)c(C2CC2)nn3-c2cc(O)c(F)c(F)c2)CC1] | 5-(3-Cyclopropyl-5-(4- (methylsulfonyl)piperazin-1-yl)-1H-indazol-1- yl)-2,3-difluorophenol |
| 21.13 | 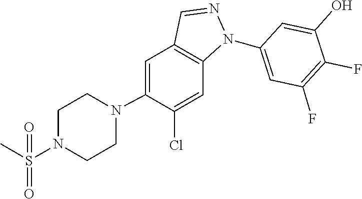 [SMILES: CS(=O)(=O)N1CCN(c2cc3cnn(-c4cc(O)c(F)c(F)c4)c3cc2Cl)CC1] | 5-(6-Chloro-5-(4-(methylsulfonyl)piperazin-1- yl)-1H-indazol-1-yl)-2,3-difluorophenol |
| 21.14 | 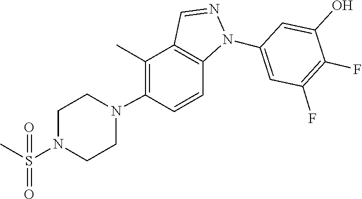 [SMILES: Cc1c(N2CCN(S(C)(=O)=O)CC2)ccc2c1cnn2-c1cc(O)c(F)c(F)c1] | 2,3-Difluoro-5-(4-methyl-5-(4- (methylsulfonyl)piperazin-1-yl)-1H-indazol-1- yl)phenol |
| 21.15 | 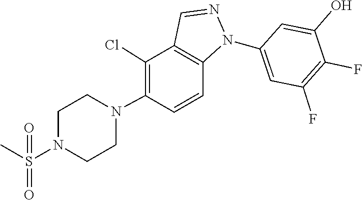 [SMILES: CS(=O)(=O)N1CCN(c2ccc3c(cnn3-c3cc(O)c(F)c(F)c3)c2Cl)CC1] | 5-(4-Chloro-5-(4-(methylsulfonyl)piperazin-1- yl)-1H-indazol-1-yl)-2,3-difluorophenol |
| 21.16 | 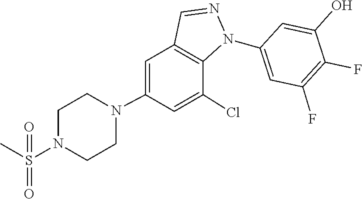 [SMILES: CS(=O)(=O)N1CCN(c2cc(Cl)c3c(cnn3-c3cc(O)c(F)c(F)c3)c2)CC1] | 5-(7-Chloro-5-(4-(methylsulfonyl)piperazin-1- yl)-1H-indazol-1-yl)-2,3-difluorophenol |
| 21.17 | 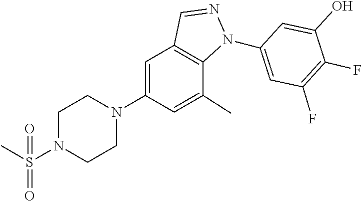 [SMILES: Cc1cc(N2CCN(S(C)(=O)=O)CC2)cc2cnn(-c3cc(O)c(F)c(F)c3)c12] | 2,3-Difluoro-5-(7-methyl-5-(4- (methylsulfonyl)piperazin-1-yl)-1H-indazol-1- yl)phenol |
| 21.18 | 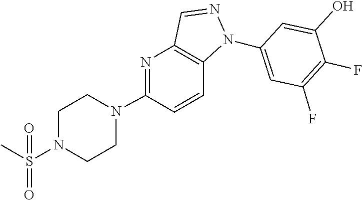 [SMILES: CS(=O)(=O)N1CCN(c2ccc3c(cnn3-c3cc(O)c(F)c(F)c3)n2)CC1] | 2,3-Difluoro-5-(5-(4-(methylsulfonyl)piperazin- 1-yl)-1H-pyrazolo[4,3-b]pyridine-1-yl)phenol |
| 21.19 | 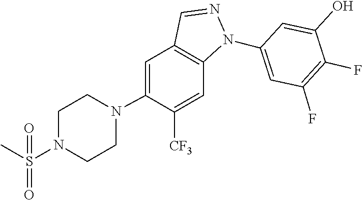 [SMILES: CS(=O)(=O)N1CCN(c2cc3cnn(-c4cc(O)c(F)c(F)c4)c3cc2C(F)(F)F)CC1] | 2,3-Difluoro-5-(5-(4-(methylsulfonyl)piperazin- 1-yl)-6-(trifluoromethyl)-1H-indazol-1- yl)phenol |
| 21.20 | 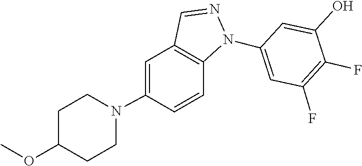 [SMILES: COC1CCN(c2ccc3c(cnn3-c3cc(O)c(F)c(F)c3)c2)CC1] | 2,3-Difluoro-5-(5-(4-methoxypiperidin-1-yl)- 1H-indazol-1-yl)phenol |
| 21.21 | 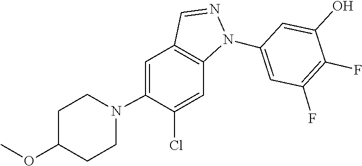 [SMILES: COC1CCN(c2cc3cnn(-c4cc(O)c(F)c(F)c4)c3cc2Cl)CC1] | 5-(6-Chloro-5-(4-methoxypiperidin-1-yl)-1H- indazol-1-yl)-2,3-difluorophenol |
| 21.22 | 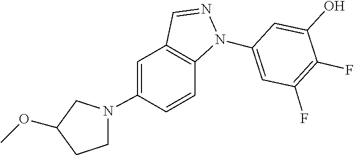 [SMILES: COC1CCN(c2ccc3c(cnn3-c3cc(O)c(F)c(F)c3)c2)C1] | 2,3-Difluoro-5-(5-(3-methoxypyrrolidin-1-yl)- 1H-indazol-1-yl)phenol |
| 21.23 | 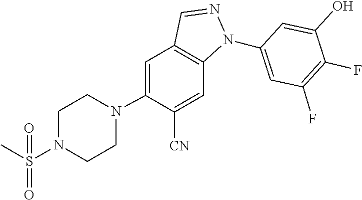 [SMILES: CS(=O)(=O)N1CCN(c2cc3cnn(-c4cc(O)c(F)c(F)c4)c3cc2C#N)CC1] | 1-(3,4-Difluoro-5-hydroxyphenyl)-5-(4- (methylsulfonyl)piperazin-1-yl)-1H-indazole-6- carbonitrile |
| 21.24 | 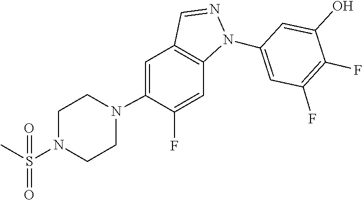 [SMILES: CS(=O)(=O)N1CCN(c2cc3cnn(-c4cc(O)c(F)c(F)c4)c3cc2F)CC1] | 2,3-Difluoro-5-(6-fluoro-5-(4- (methylsulfonyl)piperazin-1-yl)-1H-indazol-1- yl)phenol |
| 21.25 | 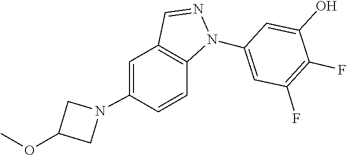 [SMILES: COC1CN(c2ccc3c(cnn3-c3cc(O)c(F)c(F)c3)c2)C1] | 2,3-Difluoro-5-(5-(3-methoxyazetidin-1-yl)-1H- indazol-1-yl)phenol |
| 21.26 | 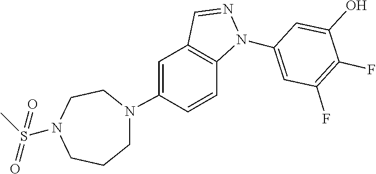 [SMILES: CS(=O)(=O)N1CCCN(c2ccc3c(cnn3-c3cc(O)c(F)c(F)c3)c2)CC1] | 2,3-Difluoro-5-(5-(4-(methylsulfonyl)-1,4- diazepan-1-yl)-1H-indazol-1-yl)phenol |
| 21.27 | 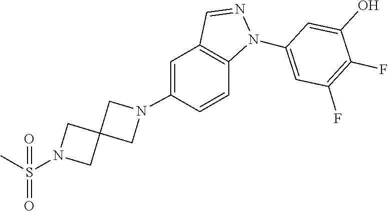 [SMILES: CS(=O)(=O)N1CC2(CN(c3ccc4c(cnn4-c4cc(O)c(F)c(F)c4)c3)C2)C1] | 2,3-Difluoro-5-(5-(6-(methylsulfonyl)-2,6- diazaspiro[3.3]heptan-2-yl)-1H-indazol-1- yl)phenol |
| 21.28 | 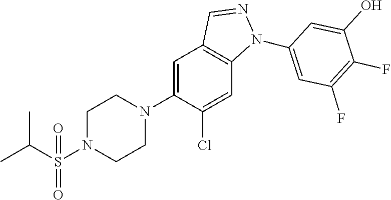 [SMILES: CC(C)S(=O)(=O)N1CCN(c2cc3cnn(-c4cc(O)c(F)c(F)c4)c3cc2Cl)CC1] | 5-(6-Chloro-5-(4-(isopropylsulfonyl)piperazin- 1-yl)-1H-indazol-1-yl)-2,3-difluorophenol |
| 21.29 | 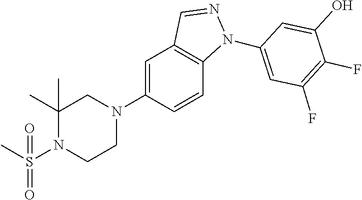 [SMILES: CC1(C)CN(c2ccc3c(cnn3-c3cc(O)c(F)c(F)c3)c2)CCN1S(C)(=O)=O] | 5-(5-(3,3-Dimethyl-4- (methylsulfonyl)piperazin-1-yl)-1H-indazol-1- yl)-2,3-difluorophenol |
| 21.30 | 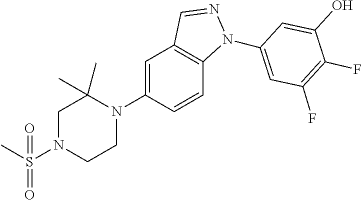 [SMILES: CC1(C)CN(S(C)(=O)=O)CCN1c1ccc2c(cnn2-c2cc(O)c(F)c(F)c2)c1] | 5-(5-(2,2-Dimethyl-4- (methylsulfonyl)piperazin-1-yl)-1H-indazol-1- yl)-2,3-difluorophenol |
| 21.31 | 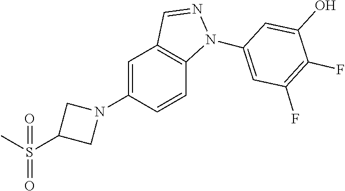 [SMILES: CS(=O)(=O)C1CN(c2ccc3c(cnn3-c3cc(O)c(F)c(F)c3)c2)C1] | 2,3-Difluoro-5-(5-(3-(methylsulfonyl)azetidin- 1-yl)-1H-indazol-1-yl)phenol |
| 21.32 | 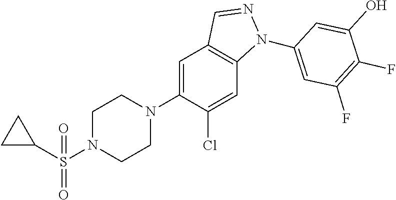 [SMILES: O=S(=O)(C1CC1)N1CCN(c2cc3cnn(-c4cc(O)c(F)c(F)c4)c3cc2Cl)CC1] | 5-(6-Chloro-5-(4- (cyclopropylsulfonyl)piperazin-1-yl)-1H- indazol-1-yl)-2,3-difluorophenol |
| 21.33 | 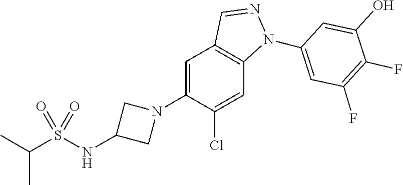 [SMILES: CC(C)S(=O)(=O)NC1CN(c2cc3cnn(-c4cc(O)c(F)c(F)c4)c3cc2Cl)C1] | N-(1-(6-Chloro-1-(3,4-difluoro-5- hydroxyphenyl)-1H-indazol-5-yl) azetidin-3- yl)propane-2-sulfonamide |
| 21.34 | 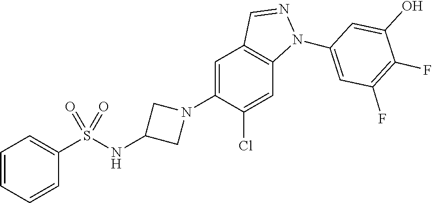 [SMILES: O=S(=O)(NC1CN(c2cc3cnn(-c4cc(O)c(F)c(F)c4)c3cc2Cl)C1)c1ccccc1] | N-(1-(6-Chloro-1-(3,4-difluoro-5- hydroxyphenyl)-1H-indazol-5-yl) azetidin-3- yl)benzenesulfonamide |
| 21.35 | 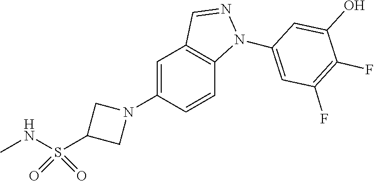 [SMILES: CNS(=O)(=O)C1CN(c2ccc3c(cnn3-c3cc(O)c(F)c(F)c3)c2)C1] | 1-(1-(3,4-Difluoro-5-hydroxyphenyl)-1H- indazol-5-yl)-N-methylazetidine-3-sulfonamide |
| 21.36 | 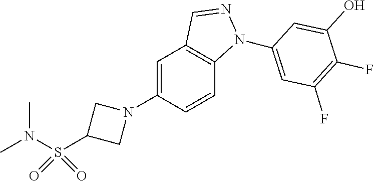 [SMILES: CN(C)S(=O)(=O)C1CN(c2ccc3c(cnn3-c3cc(O)c(F)c(F)c3)c2)C1] | 1-(1-(3,4-Difluoro-5-hydroxyphenyl)-1H- indazol-5-yl)-N,N-dimethylazetidine-3- sulfonamide |
| 21.37 | 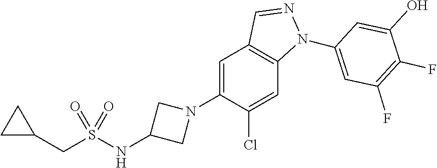 [SMILES: O=S(=O)(CC1CC1)NC1CN(c2cc3cnn(-c4cc(O)c(F)c(F)c4)c3cc2Cl)C1] | N-(1-(6-Chloro-1-(3,4-difluoro-5- hydroxyphenyl)-1H-indazol-5-yl)azetidin-3-yl)- 1-cyclopropylmethanesulfonamide |
| 21.38 | 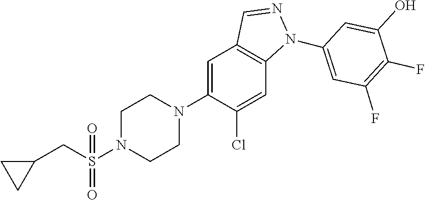 [SMILES: O=S(=O)(CC1CC1)N1CCN(c2cc3cnn(-c4cc(O)c(F)c(F)c4)c3cc2Cl)CC1] | 5-(6-Chloro-5-(4- ((cyclopropylmethyl)sulfonyl)piperazin-1-yl)- 1H-indazol-1-yl)-2,3-difluorophenol |
| 21.39 | 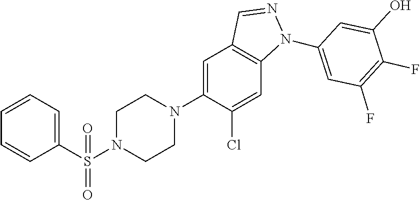 [SMILES: O=S(=O)(c1ccccc1)N1CCN(c2cc3cnn(-c4cc(O)c(F)c(F)c4)c3cc2Cl)CC1] | 5-(6-Chloro-5-(4-(phenylsulfonyl)piperazin-1- yl)-1H-indazol-1-yl)-2,3-difluorophenol |
| 21.40 | 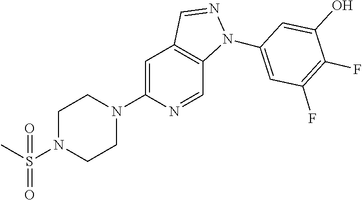 [SMILES: CS(=O)(=O)N1CCN(c2cc3cnn(-c4cc(O)c(F)c(F)c4)c3cn2)CC1] | 2,3-Difluoro-5-(5-(4-(methylsulfonyl)piperazin- 1-yl)-1H-pyrazolo[3,4-c]pyridine-1-yl)phenol |
| 21.41 | 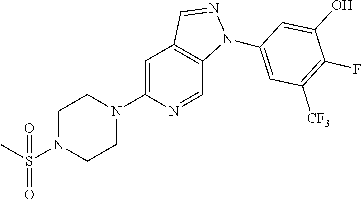 [SMILES: CS(=O)(=O)N1CCN(c2cc3cnn(-c4cc(O)c(F)c(C(F)(F)F)c4)c3cn2)CC1] | 2-Fluoro-5-(5-(4-(methylsulfonyl)piperazin-1- yl)-1H-pyrazolo[3,4-c]pyridine-1-yl)-3- (trifluoromethyl)phenol |
| 21.42 | 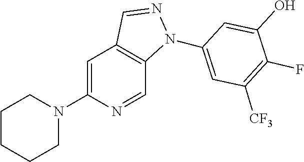 [SMILES: Oc1cc(-n2ncc3cc(N4CCCCC4)ncc32)cc(C(F)(F)F)c1F] | 2-Fluoro-5-(5-(piperidin-1-yl)-1H-pyrazolo[3,4- c]pyridine-1-yl)-3-(trifluoromethyl)phenol |
| 21.43 | 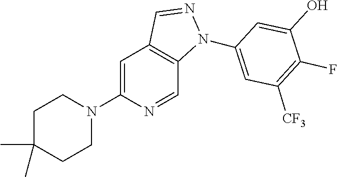 [SMILES: CC1(C)CCN(c2cc3cnn(-c4cc(O)c(F)c(C(F)(F)F)c4)c3cn2)CC1] | 5-(5-(4,4-Dimethylpiperidin-1-yl)-1H- pyrazolo[3,4-c]pyridine-1-yl)-2-fluoro-3- (trifluoromethyl)phenol |
| 21.44 | 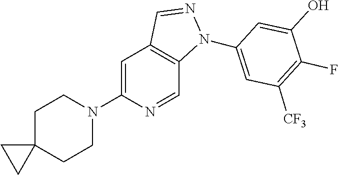 [SMILES: Oc1cc(-n2ncc3cc(N4CCC5(CC4)CC5)ncc32)cc(C(F)(F)F)c1F] | 5-(5-(6-Azaspiro[2.5]octan-6-yl)-1H- pyrazolo[3,4-c]pyridine-1-yl)-2-fluoro-3- (trifluoromethyl)phenol |
| 21.45 | 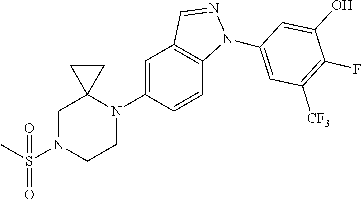 [SMILES: CS(=O)(=O)N1CCN(c2ccc3c(cnn3-c3cc(O)c(F)c(C(F)(F)F)c3)c2)C2(CC2)C1] | 2-Fluoro-5-(5-(7-(methylsulfonyl)-4,7- diazaspiro[2.5]octan-4-yl)-1H-indazol-1-yl)-3- (trifluoromethyl)phenol |
| 21.46 | 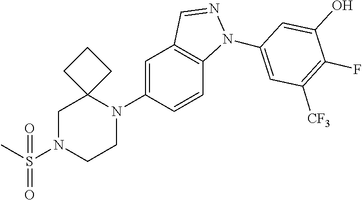 [SMILES: CS(=O)(=O)N1CCN(c2ccc3c(cnn3-c3cc(O)c(F)c(C(F)(F)F)c3)c2)C2(CCC2)C1] | 2-Fluoro-5-(5-(8-(methylsulfonyl)-5,8- diazaspiro[3.5]nonan-5-yl)-1H-indazol-1-yl)-3- (trifluoromethyl)phenol |
| 21.47 | 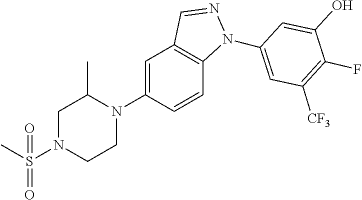 [SMILES: CC1CN(S(C)(=O)=O)CCN1c1ccc2c(cnn2-c2cc(O)c(F)c(C(F)(F)F)c2)c1] | 2-Fluoro-5-(5-(2-methyl-4- (methylsulfonyl)piperazin-1-yl)-1H-indazol-1- yl)-3-(trifluoromethyl)phenol |
| 21.48 | 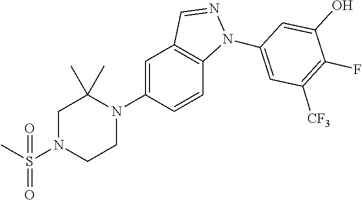 [SMILES: CC1(C)CN(S(C)(=O)=O)CCN1c1ccc2c(cnn2-c2cc(O)c(F)c(C(F)(F)F)c2)c1] | 5-(5-(2,2-Dimethyl-4- (methylsulfonyl)piperazin-1-yl)-1H-indazol-1- yl)-2-fluoro-3-(trifluoromethyl)phenol |
| 21.49 | 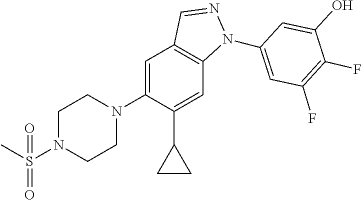 [SMILES: CS(=O)(=O)N1CCN(c2cc3cnn(-c4cc(O)c(F)c(F)c4)c3cc2C2CC2)CC1] | 5-(6-Cyclopropyl-5-(4- (methylsulfonyl)piperazin-1-yl)-1H-indazol-1- yl)-2,3-difluorophenol |
| 21.50 | 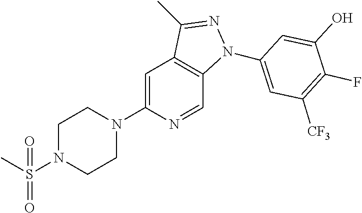 [SMILES: Cc1nn(-c2cc(O)c(F)c(C(F)(F)F)c2)c2cnc(N3CCN(S(C)(=O)=O)CC3)cc12] | 2-Fluoro-5-(3-methyl-5-(4- (methylsulfonyl)piperazin-1-yl)-1H- pyrazolo[3,4-c]pyridine-1-yl)-3- (trifluoromethyl)phenol |
| 21.51 | 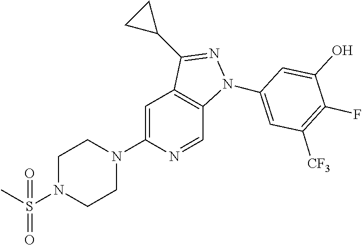 [SMILES: CS(=O)(=O)N1CCN(c2cc3c(C4CC4)nn(-c4cc(O)c(F)c(C(F)(F)F)c4)c3cn2)CC1] | 5-(3-Cyclopropyl-5-(4- (methylsulfonyl)piperazin-1-yl)-1H- pyrazolo[3,4-c]pyridine-1-yl)-2-fluoro-3- (trifluoromethyl)phenol |
| 21.52 | 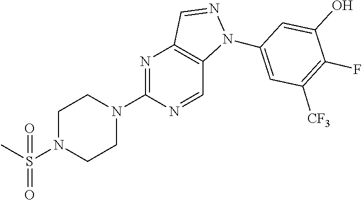 [SMILES: CS(=O)(=O)N1CCN(c2ncc3c(cnn3-c3cc(O)c(F)c(C(F)(F)F)c3)n2)CC1] | 2-Fluoro-5-(5-(4-(methylsulfonyl)piperazin-1- yl)-1H-pyrazolo[4,3-d]pyrimidin-1-yl)-3- (trifluoromethyl)phenol |
| 21.53 | 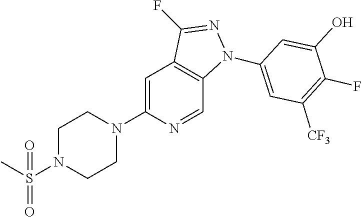 [SMILES: CS(=O)(=O)N1CCN(c2cc3c(F)nn(-c4cc(O)c(F)c(C(F)(F)F)c4)c3cn2)CC1] | 2-Fluoro-5-(3-fluoro-5-(4- (methylsulfonyl)piperazin-1-yl)-1H- pyrazolo[3,4-c]pyridin-1-yl)-3- (trifluoromethyl)phenol |
| 21.54 | 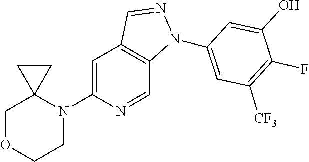 [SMILES: Oc1cc(-n2ncc3cc(N4CCOCC45CC5)ncc32)cc(C(F)(F)F)c1F] | 5-(5-(7-Oxa-4-azaspiro[2.5]octan-4-yl)-1H- pyrazolo[3,4-c]pyridin-1-yl)-2-fluoro-3- (trifluoromethyl)phenol |
| 21.55 | 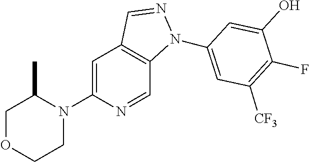 [SMILES: C[C@@H]1COCCN1c1cc2cnn(-c3cc(O)c(F)c(C(F)(F)F)c3)c2cn1] | (R)-2-Fluoro-5-(5-(3-methylmorpholino)-1H- pyrazolo[3,4-c]pyridin-1-yl)-3- (trifluoromethyl)phenol |
| 21.56 | 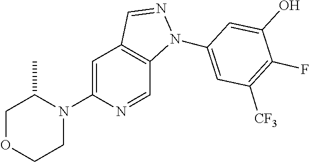 [SMILES: C[C@H]1COCCN1c1cc2cnn(-c3cc(O)c(F)c(C(F)(F)F)c3)c2cn1] | (S)-2-Fluoro-5-(5-(3-methylmorpholino)-1H- pyrazolo[3,4-c]pyridin-1-yl)-3- (trifluoromethyl)phenol |
| 21.57 | 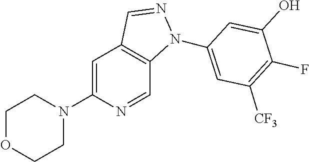 [SMILES: Oc1cc(-n2ncc3cc(N4CCOCC4)ncc32)cc(C(F)(F)F)c1F] | 2-Fluoro-5-(5-morpholino-1H-pyrazolo[3,4- c]pyridin-1-yl)-3-(trifluoromethyl)phenol |
| 21.58 | 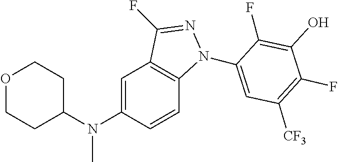 [SMILES: CN(c1ccc2c(c1)c(F)nn2-c1cc(C(F)(F)F)c(F)c(O)c1F)C1CCOCC1] | 2,6-Difluoro-3-(3-fluoro-5-(methyl(tetrahydro- 2H-pyran-4-yl)amino)-1H-indazol-1-yl)-5- (trifluoromethyl)phenol |
| 21.59 | 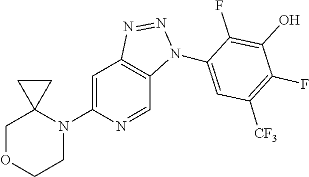 [SMILES: Oc1c(F)c(-n2nnc3cc(N4CCOCC45CC5)ncc32)cc(C(F)(F)F)c1F] | 3-(6-(7-Oxa-4-azaspiro[2.5]octan-4-yl)-3H- [1,2,3]triazolo[4,5-c]pyridin-3-yl)-2,6-difluoro- 5-(trifluoromethyl)phenol |
| 21.60 | 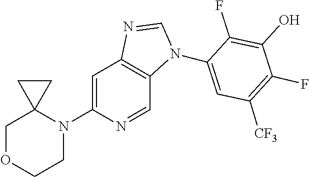 [SMILES: Oc1c(F)c(-n2cnc3cc(N4CCOCC45CC5)ncc32)cc(C(F)(F)F)c1F] | 3-(6-(7-Oxa-4-azaspiro[2.5]octan-4-yl)-3H- imidazo[4,5-c]pyridin-3-yl)-2,6-difluoro-5- (trifluoromethyl)phenol |
| 21.61 | 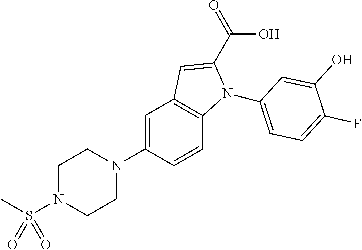 [SMILES: CS(=O)(=O)N1CCN(c2ccc3c(c2)cc(C(=O)O)n3-c2ccc(F)c(O)c2)CC1] | 1-(4-Fluoro-3-hydroxyphenyl)-5-(4- (methylsulfonyl)piperazin-1-yl)-1H-indole-2- carboxylic acid |
| 21.62 | 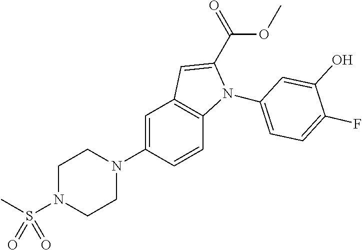 [SMILES: COC(=O)c1cc2cc(N3CCN(S(C)(=O)=O)CC3)ccc2n1-c1ccc(F)c(O)c1] | Methyl 1-(4-fluoro-3-hydroxyphenyl)-5-(4- (methylsulfonyl)piperazin-1-yl)-1H-indole-2- carboxylate |
| 21.63 | 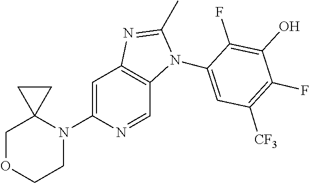 [SMILES: Cc1nc2cc(N3CCOCC34CC4)ncc2n1-c1cc(C(F)(F)F)c(F)c(O)c1F] | 2,6-Difluoro-3-(2-methyl-6-(7-oxa-4- azaspiro[2.5]octan-4-yl)-3H-imidazo[4,5- c]pyridin-3-yl)-5-(trifluoromethyl)phenol |
| 22 | 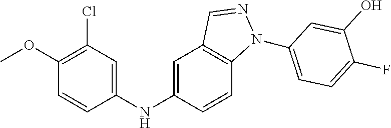 [SMILES: COc1ccc(Nc2ccc3c(cnn3-c3ccc(F)c(O)c3)c2)cc1Cl] | 5-(5-((3-Chloro-4-methoxyphenyl)amino)-1H- indazol-1-yl)-2-fluorophenol |
| 23 | 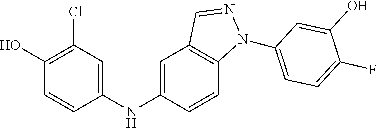 [SMILES: Oc1cc(-n2ncc3cc(Nc4ccc(O)c(Cl)c4)ccc32)ccc1F] | 2-Chloro-4-((1-(4-fluoro-3-hydroxyphenyl)-1H- indazol-5-yl)amino)phenol |
| 24 | 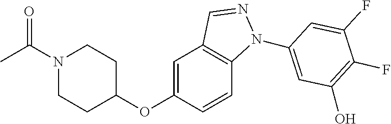 [SMILES: CC(=O)N1CCC(Oc2ccc3c(cnn3-c3cc(O)c(F)c(F)c3)c2)CC1] | 1-(4-((1-(3,4-Difluoro-5-hydroxyphenyl)-1H- indazol-5-yl)oxy)piperidin-1-yl)ethan-1-one |
| 24.01 | 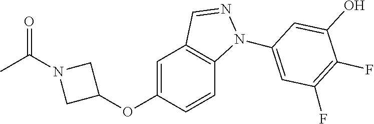 [SMILES: CC(=O)N1CC(Oc2ccc3c(cnn3-c3cc(O)c(F)c(F)c3)c2)C1] | 1-(3-((1-(3,4-Difluoro-5-hydroxyphenyl)-1H- indazol-5-yl)oxy )azetidin-1-yl)ethan-1-one |
| 25 | 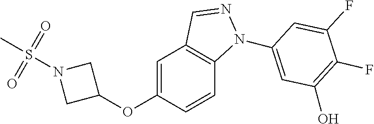 [SMILES: CS(=O)(=O)N1CC(Oc2ccc3c(cnn3-c3cc(O)c(F)c(F)c3)c2)C1] | 2,3-Difluoro-5-(5-((1-(methylsulfonyl)azetidin- 3-yl)oxy)-1H-indazol-1-yl)phenol |
| 25.01 | 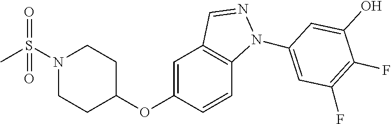 [SMILES: CS(=O)(=O)N1CCC(Oc2ccc3c(cnn3-c3cc(O)c(F)c(F)c3)c2)CC1] | 2,3-Difluoro-5-(5-((1- (methylsulfonyl)piperidin-4-yl)oxy)-1H- indazol-1-yl)phenol |
| 26 | 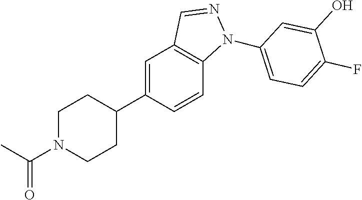 [SMILES: CC(=O)N1CCC(c2ccc3c(cnn3-c3ccc(F)c(O)c3)c2)CC1] | 1-(4-(1-(4-Fluoro-3-hydroxyphenyl)-1H- indazol-5-yl)piperidin-1-yl)ethanone |
| 26.01 | 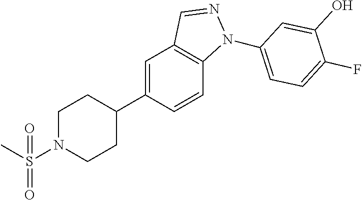 [SMILES: CS(=O)(=O)N1CCC(c2ccc3c(cnn3-c3ccc(F)c(O)c3)c2)CC1] | 2-Fluoro-5-(5-(1-(methylsulfonyl)piperidin-4- yl)-1H-indazol-1-yl)phenol |
| 26.02 | 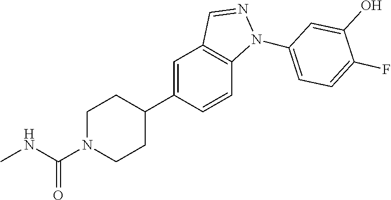 [SMILES: CNC(=O)N1CCC(c2ccc3c(cnn3-c3ccc(F)c(O)c3)c2)CC1] | 4-(1-(4-Fluoro-3-hydroxyphenyl)-1H-indazol-5- yl)-N-methylpiperidine-1-carboxamide |
| 27 | 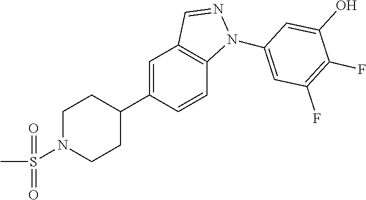 [SMILES: CS(=O)(=O)N1CCC(c2ccc3c(cnn3-c3cc(O)c(F)c(F)c3)c2)CC1] | 2,3-Difluoro-5-(5-(1-(methylsulfonyl)piperidin- 4-yl)-1H-indazol-1-yl)phenol |
| 27.01 | 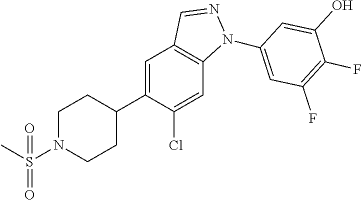 [SMILES: CS(=O)(=O)N1CCC(c2cc3cnn(-c4cc(O)c(F)c(F)c4)c3cc2Cl)CC1] | 5-(6-Chloro-5-(1-(methylsulfonyl)piperidin-4- yl)-1H-indazol-1-yl)-2,3-difluorophenol |
| 28 | 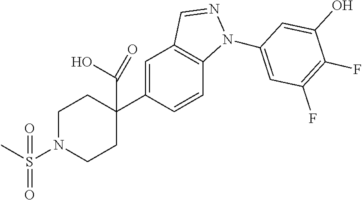 [SMILES: CS(=O)(=O)N1CCC(C(=O)O)(c2ccc3c(cnn3-c3cc(O)c(F)c(F)c3)c2)CC1] | 4-(1-(3,4-Difluoro-5-hydroxyphenyl)-1H- indazol-5-yl)-1-(methylsulfonyl)piperidine-4- carboxylic acid |
| 28.01 | 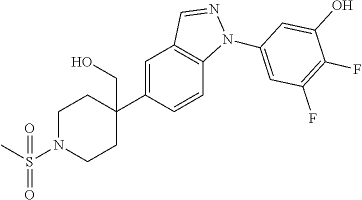 [SMILES: CS(=O)(=O)N1CCC(CO)(c2ccc3c(cnn3-c3cc(O)c(F)c(F)c3)c2)CC1] | 2,3-Difluoro-5-(5-(4-(hydroxymethyl)-1- (methylsulfonyl)piperidin-4-yl)-1H-indazol-1- yl)phenol |
| 28.02 | 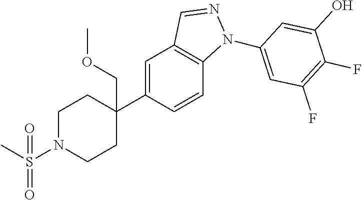 [SMILES: COCC1(c2ccc3c(cnn3-c3cc(O)c(F)c(F)c3)c2)CCN(S(C)(=O)=O)CC1] | 2,3-Difluoro-5-(5-(4-(methoxymethyl)-1- (methylsulfonyl)piperidin-4-yl)-1H-indazol-1- yl)phenol |
| 28.03 | 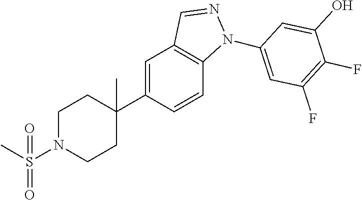 [SMILES: CC1(c2ccc3c(cnn3-c3cc(O)c(F)c(F)c3)c2)CCN(S(C)(=O)=O)CC1] | 2,3-Difluoro-5-(5-(4-methyl-1- (methylsulfonyl)piperidin-4-yl)-1H-indazol-1- yl)phenol |
| 28.04 | 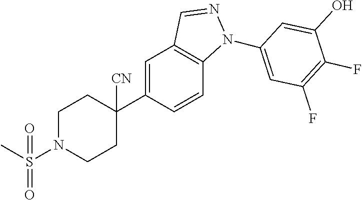 [SMILES: CS(=O)(=O)N1CCC(C#N)(c2ccc3c(cnn3-c3cc(O)c(F)c(F)c3)c2)CC1] | 4-(1-(3,4-Difluoro-5-hydroxyphenyl)-1H- indazol-5-yl)-1-(methylsulfonyl)piperidine-4- carbonitrile |
| 28.05 | 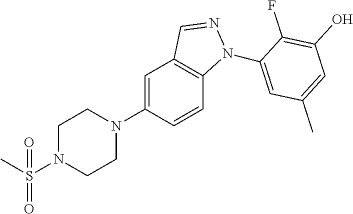 [SMILES: Cc1cc(O)c(F)c(-n2ncc3cc(N4CCN(S(C)(=O)=O)CC4)ccc32)c1] | 2-Fluoro-5-methyl-3-(5-(4- (methylsulfonyl)piperazin-1-yl)-1H-indazol-1- yl)phenol |
| 28.06 | 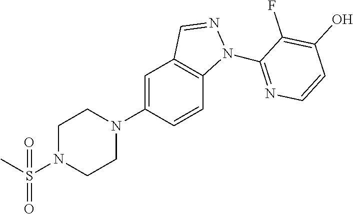 [SMILES: CS(=O)(=O)N1CCN(c2ccc3c(cnn3-c3nccc(O)c3F)c2)CC1] | 3-Fluoro-2-(5-(4-(methylsulfonyl)piperazin-1- yl)-1H-indazol-1-yl)pyridin-4-ol |
| 28.07 | 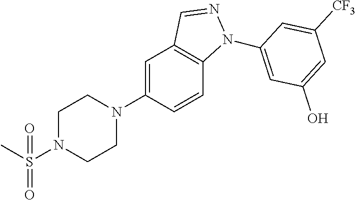 [SMILES: CS(=O)(=O)N1CCN(c2ccc3c(cnn3-c3cc(O)cc(C(F)(F)F)c3)c2)CC1] | 3-(5-(4-(Methylsulfonyl)piperazin-1-yl)-1H- indazol-1-yl)-5-(trifluoromethyl)phenol |
| 28.08 | 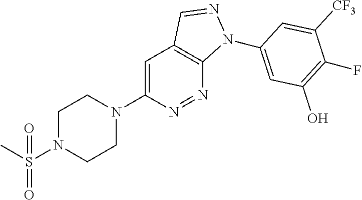 [SMILES: CS(=O)(=O)N1CCN(c2cc3cnn(-c4cc(O)c(F)c(C(F)(F)F)c4)c3nn2)CC1] | 2-Fluoro-5-(5-(4-(methylsulfonyl)piperazin-1- yl)-1H-pyrazolo[3,4-c]pyridazin-1-yl)-3- (trifluoromethyl)phenol |
| 29 | 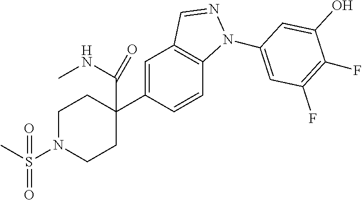 [SMILES: CNC(=O)C1(c2ccc3c(cnn3-c3cc(O)c(F)c(F)c3)c2)CCN(S(C)(=O)=O)CC1] | 4-(1-(3,4-Difluoro-5-hydroxyphenyl)-1H- indazol-5-yl)-N-methyl-1- (methylsulfonyl)piperidine-4-carboxamide |
| 29.01 | 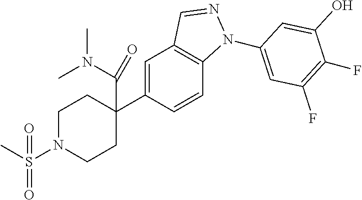 [SMILES: CN(C)C(=O)C1(c2ccc3c(cnn3-c3cc(O)c(F)c(F)c3)c2)CCN(S(C)(=O)=O)CC1] | 4-(1-(3,4-Difluoro-5-hydroxyphenyl)-1H- indazol-5-yl)-N,N-dimethyl-1- (methylsulfonyl)piperidine-4-carboxamide |
| 30 | 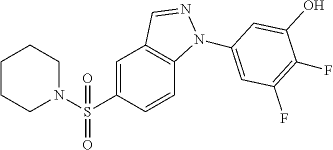 [SMILES: O=S(=O)(c1ccc2c(cnn2-c2cc(O)c(F)c(F)c2)c1)N1CCCCC1] | 2,3-Difluoro-5-(5-(piperidin-1-ylsulfonyl)-1H- indazol-1-yl)phenol |
| 30.01 | 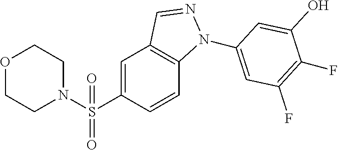 [SMILES: O=S(=O)(c1ccc2c(cnn2-c2cc(O)c(F)c(F)c2)c1)N1CCOCC1] | 2,3-Difluoro-5-(5-(morpholinosulfonyl)-1H- indazol-1-yl)phenol |
| 31 | 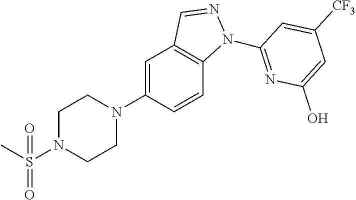 [SMILES: CS(=O)(=O)N1CCN(c2ccc3c(cnn3-c3cc(C(F)(F)F)cc(O)n3)c2)CC1] | 6-(5-(4-(Methylsulfonyl)piperazin-1-yl)-1H- indazol-1-yl)-4-(trifluoromethyl)pyridin-2-ol |
| 31.01 | 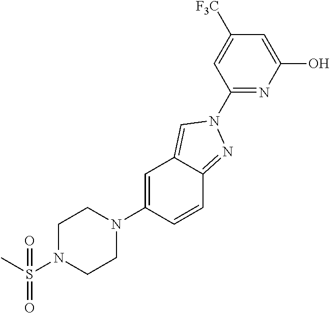 [SMILES: CS(=O)(=O)N1CCN(c2ccc3nn(-c4cc(C(F)(F)F)cc(O)n4)cc3c2)CC1] | 6-(5-(4-(Methylsulfonyl)piperazin-1-yl)-2H- indazol-2-yl)-4-(trifluoromethyl)pyridin-2-ol |
| 31.02 | 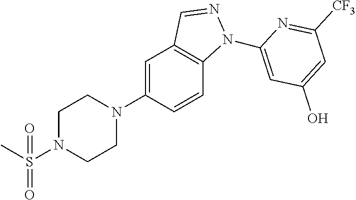 [SMILES: CS(=O)(=O)N1CCN(c2ccc3c(cnn3-c3cc(O)cc(C(F)(F)F)n3)c2)CC1] | 2-(5-(4-(Methylsulfonyl)piperazin-1-yl)-1H- indazol-1-yl)-6-(trifluoromethyl)pyridin-4-ol |
| 31.03 | 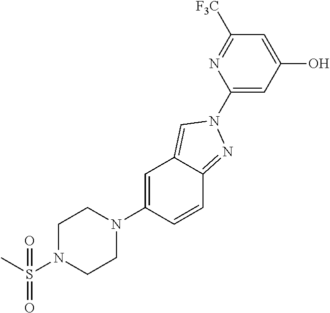 [SMILES: CS(=O)(=O)N1CCN(c2ccc3nn(-c4cc(O)cc(C(F)(F)F)n4)cc3c2)CC1] | 2-(5-(4-(Methylsulfonyl)piperazin-1-yl)-2H- indazol-2-yl)-6-(trifluoromethyl)pyridin-4-ol |

In some embodiments, provided herein is a pharmaceutically acceptable salt or solvate of a compound that is described in Table 1.

In one aspect, compounds described herein are in the form of pharmaceutically acceptable salts. As well, active metabolites of these compounds having the same type of activity are included in the scope of the present disclosure. In addition, the compounds described herein can exist in unsolvated as well as solvated forms with pharmaceutically acceptable solvents such as water, ethanol, and the like. The solvated forms of the compounds presented herein are also considered to be disclosed herein.

“Pharmaceutically acceptable,” as used herein, refers a material, such as a carrier or diluent, which does not abrogate the biological activity or properties of the compound, and is relatively nontoxic, i.e., the material is administered to an individual without causing undesirable biological effects or interacting in a deleterious manner with any of the components of the composition in which it is contained.

The term “pharmaceutically acceptable salt” refers to a form of a therapeutically active agent that consists of a cationic form of the therapeutically active agent in combination with a suitable anion, or in alternative embodiments, an anionic form of the therapeutically active agent in combination with a suitable cation. Handbook of Pharmaceutical Salts: Properties, Selection and Use. International Union of Pure and Applied Chemistry, Wiley—VCH 2002. S. M. Berge, L. D. Bighley, D. C. Monkhouse, J. Pharm. Sci. 1977, 66, 1-19. P. H. Stahl and C. G. Wermuth, editors, Handbook of Pharmaceutical Salts: Properties, Selection and Use, Weinheim/Zürich: Wiley-VCH/VHCA, 2002. Pharmaceutical salts typically are more soluble and more rapidly soluble in stomach and intestinal fluids than non-ionic species and so are useful in solid dosage forms. Furthermore, because their solubility often is a function of pH, selective dissolution in one or another part of the digestive tract is possible, and this capability can be manipulated as one aspect of delayed and sustained release behaviors. Also, because the salt-forming molecule can be in equilibrium with a neutral form, passage through biological membranes can be adjusted.

In some embodiments, pharmaceutically acceptable salts are obtained by reacting a compound described herein with an acid to provide a “pharmaceutically acceptable acid addition salt.” In some embodiments, the compound described herein (i.e. free base form) is basic and is reacted with an organic acid or an inorganic acid. Inorganic acids include, but are not limited to, hydrochloric acid, hydrobromic acid, sulfuric acid, phosphoric acid, nitric acid, and metaphosphoric acid. Organic acids include, but are not limited to, 1-hydroxy-2-naphthoic acid; 2,2-dichloroacetic acid; 2-hydroxy ethanesulfonic acid; 2-oxoglutaric acid; 4-acetamidobenzoic acid; 4-aminosalicylic acid; acetic acid; adipic acid; ascorbic acid (L); aspartic acid (L); benzenesulfonic acid; benzoic acid; camphoric acid (+); camphor-10-sulfonic acid (+); capric acid (decanoic acid); caproic acid (hexanoic acid); caprylic acid (octanoic acid); carbonic acid; cinnamic acid; citric acid; cyclamic acid; dodecylsulfuric acid; ethane-1,2-disulfonic acid; ethanesulfonic acid; formic acid; fumaric acid; galactaric acid; gentisic acid; glucoheptonic acid (D); gluconic acid (D); glucuronic acid (D); glutamic acid; glutaric acid; glycerophosphoric acid; glycolic acid; hippuric acid; isobutyric acid; lactic acid (DL); lactobionic acid; lauric acid; maleic acid; malic acid (−L); malonic acid; mandelic acid (DL); methanesulfonic acid; monomethyl fumarate, naphthalene-1,5-disulfonic acid; naphthalene-2-sulfonic acid; nicotinic acid; oleic acid; oxalic acid; palmitic acid; pamoic acid; phosphoric acid; proprionic acid; pyroglutamic acid (−L); salicylic acid; sebacic acid; stearic acid; succinic acid; sulfuric acid; tartaric acid (+L); thiocyanic acid; toluenesulfonic acid (p); and undecylenic acid.

In some embodiments, a compound described herein is prepared as a chloride salt, sulfate salt, bromide salt, mesylate salt, maleate salt, citrate salt or phosphate salt.

In some embodiments, pharmaceutically acceptable salts are obtained by reacting a compound described herein with abase to provide a “pharmaceutically acceptable base addition salt.”

In some embodiments, the compound described herein is acidic and is reacted with a base. In such situations, an acidic proton of the compound described herein is replaced by a metal ion, e.g., lithium, sodium, potassium, magnesium, calcium, or an aluminum ion. In some cases, compounds described herein coordinate with an organic base, such as, but not limited to, ethanolamine, diethanolamine, triethanolamine, tromethamine, meglumine, N-methylglucamine, dicyclohexylamine, tris(hydroxymethyl)methylamine. In other cases, compounds described herein form salts with amino acids such as, but not limited to, arginine, lysine, and the like. Acceptable inorganic bases used to form salts with compounds that include an acidic proton, include, but are not limited to, aluminum hydroxide, calcium hydroxide, potassium hydroxide, sodium carbonate, potassium carbonate, sodium hydroxide, lithium hydroxide, and the like. In some embodiments, the compounds provided herein are prepared as a sodium salt, calcium salt, potassium salt, magnesium salt, meglumine salt, N-methylglucamine salt or ammonium salt.

It should be understood that a reference to a pharmaceutically acceptable salt includes the solvent addition forms. In some embodiments, solvates contain either stoichiometric or non-stoichiometric amounts of a solvent, and are formed during the process of isolating or purifying the compound with pharmaceutically acceptable solvents such as water, ethanol, and the like. Hydrates are formed when the solvent is water, or alcoholates are formed when the solvent is alcohol. Solvates of compounds described herein are conveniently prepared or formed during the processes described herein. In addition, the compounds provided herein optionally exist in unsolvated as well as solvated forms.

The methods and formulations described herein include the use of N-oxides (if appropriate), crystalline forms (also known as polymorphs), or pharmaceutically acceptable salts of compounds described herein, as well as active metabolites of these compounds having the same type of activity.

In some embodiments, sites on the organic groups (e.g., alkyl groups, aromatic rings) of compounds described herein are susceptible to various metabolic reactions. Incorporation of appropriate substituents on the organic groups will reduce, minimize or eliminate this metabolic pathway. In specific embodiments, the appropriate substituentto decrease or eliminate the susceptibility of the aromatic ring to metabolic reactions is, by way of example only, a halogen, deuterium, an alkyl group, a haloalkyl group, or a deuteroalkyl group.

In another embodiment, the compounds described herein are labeled isotopically (e.g., with a radioisotope) or by another other means, including, but not limited to, the use of chromophores or fluorescent moieties, bioluminescent labels, or chemiluminescent labels.

Compounds described herein include isotopically-labeled compounds, which are identical to those recited in the various formulae and structures presented herein, but for the fact that one or more atoms are replaced by an atom having an atomic mass or mass number different from the atomic mass or mass number usually found in nature. Examples of isotopes that can be incorporated into the present compounds include isotopes of hydrogen, carbon, nitrogen, oxygen, fluorine and chlorine, such as, for example, ²H, ³H, ¹³C, ¹⁴C, ¹⁵N, ¹⁸O, ¹⁷O, ³⁵S, ¹⁸F, ³⁶Cl. In one aspect, isotopically-labeled compounds described herein, for example those into which radioactive isotopes such as ³H and ¹⁴C are incorporated, are useful in drug and/or substrate tissue distribution assays. In one aspect, substitution with isotopes such as deuterium affords certain therapeutic advantages resulting from greater metabolic stability, such as, for example, increased in vivo half-life or reduced dosage requirements. In some embodiments, one or more hydrogen atoms of the compounds described herein is replaced with deuterium.

In some embodiments, the compounds described herein possess one or more stereocenters and each stereocenter exists independently in either the R or S configuration. The compounds presented herein include all diastereomeric, enantiomeric, atropisomers, and epimeric forms as well as the appropriate mixtures thereof. The compounds and methods provided herein include all cis, trans, syn, anti, entgegen (E), and zusammen (Z) isomers as well as the appropriate mixtures thereof.

Individual stereoisomers are obtained, if desired, by methods such as, stereoselective synthesis and/or the separation of stereoisomers by chiral chromatographic columns. In certain embodiments, compounds described herein are prepared as their individual stereoisomers by reacting a racemic mixture of the compound with an optically active resolving agent to form a pair of diastereoisomeric compounds/salts, separating the diastereomers and recovering the optically pure enantiomers. In some embodiments, resolution of enantiomers is carried out using covalent diastereomeric derivatives of the compounds described herein. In another embodiment, diastereomers are separated by separation/resolution techniques based upon differences in solubility. In other embodiments, separation of stereoisomers is performed by chromatography or by the forming diastereomeric salts and separation by recrystallization, or chromatography, or any combination thereof. Jean Jacques, Andre Collet, Samuel H. Wilen, “Enantiomers, Racemates and Resolutions”, John Wiley and Sons, Inc., 1981. In some embodiments, stereoisomers are obtained by stereoselective synthesis.

In some embodiments, compounds described herein are prepared as prodrugs. A “prodrug” refers to an agent that is converted into the parent drug in vivo. Prodrugs are often useful because, in some situations, they are easier to administer than the parent drug. They are, for instance, bioavailable by oral administration whereas the parent is not. The prodrug may be a substrate for a transporter. Further or alternatively, the prodrug also has improved solubility in pharmaceutical compositions over the parent drug. In some embodiments, the design of a prodrug increases the effective water solubility. An example, without limitation, of a prodrug is a compound described herein, which is administered as an ester (the “prodrug”) but then is metabolically hydrolyzed to provide the active entity. A further example of a prodrug is a short peptide (polyaminoacid) bonded to an acid group where the peptide is metabolized to reveal the active moiety. In certain embodiments, upon in vivo administration, a prodrug is chemically converted to the biologically, pharmaceutically, or therapeutically active form of the compound. In certain embodiments, a prodrug is enzymatically metabolized by one or more steps or processes to the biologically, pharmaceutically or therapeutically active form of the compound.

Prodrugs of the compounds described herein include, but are not limited to, esters, ethers, carbonates, thiocarbonates, N-acyl derivatives, N-acyloxyalkyl derivatives, quaternary derivatives of tertiary amines, N-Mannich bases, Schiff bases, amino acid conjugates, phosphate esters, and sulfonate esters. See for example Design of Prodrugs, Bundgaard, A. Ed., Elseview, 1985 and Method in Enzymology, Widder, K. et al., Ed.; Academic, 1985, vol. 42, p. 309-396; Bundgaard, H. “Design and Application of Prodrugs” in A Textbook of Drug Design and Development, Krosgaard-Larsen and H. Bundgaard, Ed., 1991, Chapter 5, p. 113-191; and Bundgaard, H., Advanced Drug Delivery Review, 1992, 8, 1-38, each of which is incorporated herein by reference. In some embodiments, a hydroxyl group in the compounds disclosed herein is used to form a prodrug, wherein the hydroxyl group is incorporated into an acyloxyalkyl ester, alkoxycarbonyloxyalkyl ester, alkyl ester, aryl ester, phosphate ester, sugar ester, ether, and the like. In some embodiments, a hydroxyl group in the compounds disclosed herein is a prodrug wherein the hydroxyl is then metabolized in vivo to provide a carboxylic acid group. In some embodiments, a carboxyl group is used to provide an ester or amide (i.e. the prodrug), which is then metabolized in vivo to provide a carboxylic acid group. In some embodiments, compounds described herein are prepared as alkyl ester prodrugs.

Prodrug forms of the herein described compounds, wherein the prodrug is metabolized in vivo to produce a compound described herein as set forth herein are included within the scope of the claims. In some cases, some of the herein-described compounds is a prodrug for another derivative or active compound. In some embodiments, a prodrug of the compound disclosed herein permits targeted delivery of the compound to a particular region of the gastrointestinal tract. Formation of a pharmacologically active metabolite by the colonic metabolism of drugs is a commonly used “prodrug” approach for the colon-specific drug delivery systems.

In some embodiments, a prodrug is formed by the formation of a covalent linkage between drug and a carrier in such a manner that upon oral administration the moiety remains intact in the stomach and small intestine. This approach involves the formation of a prodrug, which is a pharmacologically inactive derivative of a parent drug molecule that requires spontaneous or enzymatic transformation in the biological environment to release the active drug. Formation of prodrugs has improved delivery properties over the parent drug molecule. The problem of stability of certain drugs from the adverse environment of the upper gastrointestinal tract can be eliminated by prodrug formation, which is converted into the parent drug molecule once it reaches the colon. Site specific drug delivery through site specific prodrug activation may be accomplished by the utilization of some specific property at the target site, such as altered pH or high activity of certain enzymes relative to the non-target tissues for the prodrug-drug conversion.

In some embodiments, covalent linkage of the drug with a carrier forms a conjugate. Such conjugates include, but are not limited to, azo bond conjugates, glycoside conjugates, glucuronide conjugates, cyclodextrin conjugates, dextran conjugates or amino-acid conjugates.

In additional or further embodiments, the compounds described herein are metabolized upon administration to an organism in need to produce a metabolite that is then used to produce a desired effect, including a desired therapeutic effect.

A “metabolite” of a compound disclosed herein is a derivative of that compound that is formed when the compound is metabolized. The term “active metabolite” refers to a biologically active derivative of a compound that is formed when the compound is metabolized. The term “metabolized,” as used herein, refers to the sum of the processes (including, but not limited to, hydrolysis reactions and reactions catalyzed by enzymes) by which a particular substance is changed by an organism. Thus, enzymes may produce specific structural alterations to a compound. For example, cytochrome P450 catalyzes a variety of oxidative and reductive reactions while uridine diphosphate glucuronyltransferases catalyze the transfer of an activated glucuronic-acid molecule to aromatic alcohols, aliphatic alcohols, carboxylic acids, amines and free sulphydryl groups. Metabolites of the compounds disclosed herein are optionally identified either by administration of compounds to a host and analysis of tissue samples from the host, or by incubation of compounds with hepatic cells in vitro and analysis of the resulting compounds.

In additional or further embodiments, the compounds are rapidly metabolized in plasma.

In additional or further embodiments, the compounds are rapidly metabolized by the intestines.

In additional or further embodiments, the compounds are rapidly metabolized by the liver.

### Synthesis of Compounds

Compounds described herein are synthesized using standard synthetic techniques or using methods known in the art in combination with methods described herein.

Unless otherwise indicated, conventional methods of mass spectroscopy, NMR, HPLC, protein chemistry, biochemistry, recombinant DNA techniques and pharmacology are employed.

Compounds are prepared using standard organic chemistry techniques such as those described in, for example, March's Advanced Organic Chemistry, 6^{th} Edition, John Wiley and Sons, Inc. Alternative reaction conditions for the synthetic transformations described herein may be employed such as variation of solvent, reaction temperature, reaction time, as well as different chemical reagents and other reaction conditions. The starting materials are available from commercial sources or are readily prepared.

Suitable reference books and treatise that detail the synthesis of reactants useful in the preparation of compounds described herein, or provide references to articles that describe the preparation, include for example, “Synthetic Organic Chemistry”, John Wiley & Sons, Inc., New York; S. R. Sandler et al., “Organic Functional Group Preparations,” 2nd Ed., Academic Press, New York, 1983; H. O. House, “Modem Synthetic Reactions”, 2nd Ed., W. A. Benjamin, Inc. Menlo Park, Calif 1972; T. L. Gilchrist, “Heterocyclic Chemistry”, 2nd Ed., John Wiley & Sons, New York, 1992; J. March, “Advanced Organic Chemistry: Reactions, Mechanisms and Structure”, 4th Ed., Wiley-Interscience, New York, 1992. Additional suitable reference books and treatise that detail the synthesis of reactants useful in the preparation of compounds described herein, or provide references to articles that describe the preparation, include for example, Fuhrhop, J. and Penzlin G. “Organic Synthesis: Concepts, Methods, Starting Materials”, Second, Revised and Enlarged Edition (1994) John Wiley & Sons ISBN: 3-527-29074-5; Hoffman, R. V. “Organic Chemistry, An Intermediate Text” (1996) Oxford University Press, ISBN 0-19-509618-5; Larock, R. C. “Comprehensive Organic Transformations: A Guide to Functional Group Preparations” 2nd Edition (1999) Wiley—VCH, ISBN: 0-471-19031-4; March, J. “Advanced Organic Chemistry: Reactions, Mechanisms, and Structure” 4th Edition (1992) John Wiley & Sons, ISBN: 0-471-60180-2; Otera, J. (editor) “Modern Carbonyl Chemistry” (2000) Wiley-VCH, ISBN: 3-527-29871-1; Patai, S. “Patai's 1992 Guide to the Chemistry of Functional Groups” (1992) Interscience ISBN: 0-471-93022-9; Solomons, T. W. G. “Organic Chemistry” 7th Edition (2000) John Wiley & Sons, ISBN: 0-471-19095-0; Stowell, J. C., “Intermediate Organic Chemistry” 2nd Edition (1993) Wiley-Interscience, ISBN: 0-471-57456-2; “Industrial Organic Chemicals: Starting Materials and Intermediates: An Ullmann's Encyclopedia” (1999) John Wiley & Sons, ISBN: 3-527-29645-X, in 8 volumes; “Organic Reactions” (1942-2000) John Wiley & Sons, in over 55 volumes; and “Chemistry of Functional Groups” John Wiley & Sons, in 73 volumes.

The compounds described herein are prepared by the general synthetic routes described below in Schemes 1 to 6.

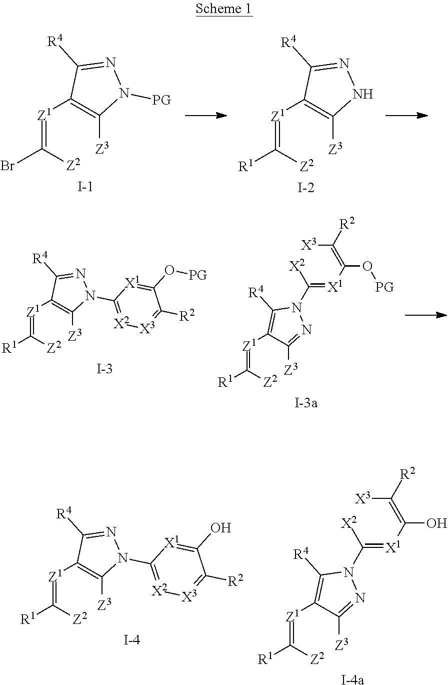

In some embodiments, compounds described herein are prepared as outlined in Scheme 1.

In some embodiments, where R¹ is an aryl or heteroaryl ring system, intermediate I-1 is reacted under appropriate Suzuki coupling reaction conditions followed by removal of a suitable protecting group to provide compound I-2. In some embodiments, appropriate Suzuki conditions include using an appropriate catalyst and boronic acid or boronic ester with an appropriate base and an appropriate solvent at an appropriate time and at an appropriate temperature. In some embodiments, the appropriate catalyst is tetrakis(triphenylphosphine)palladium(0). In some embodiments, the appropriate base is sodium carbonate. In some embodiments, the appropriate solvent mixture is dioxane:water. In some embodiments, the suitable temperature is 90° C. and the appropriate amount of time stirred is about 100 minutes.

In some embodiments, an appropriate protecting group is a tetrahydropyran protecting group. In some embodiments, appropriate conditions to remove a tetrahydropyran protecting group include using an appropriate reagent in an appropriate solvent at an appropriate temperature and amount of time. In some embodiments, the appropriate reagent is hydrogen chloride. In some embodiments, the appropriate solvent is diethylether. In some embodiments, the appropriate temperature is room temperature and the appropriate amount of time is overnight.

In some embodiments, intermediate I-2 is reacted with an appropriate aryl-halide under appropriate Ullmann coupling reaction conditions using an appropriate catalyst and catalyst ligand and an appropriate base in an appropriate solvent or solvent mixture at an appropriate temperature and appropriate amount of time to give intermediates I-3 and I-3a. In some embodiments, a suitable aryl-halide is an aryl-iodide. In some embodiments, the appropriate catalyst is copper iodide. In some embodiments, the appropriate catalyst ligand is N1,N2-dimethylethane-1,2-diamine. In some embodiments, the appropriate base is potassium phosphate. In some embodiments, the appropriate solvent is DMF. In some embodiments, the suitable temperature is 85° C. and the appropriate amount of time is about 2 days.

In some embodiments, intermediate I-2 is reacted with an appropriate boronic acid under appropriate Chan-Lam coupling reaction conditions using an appropriate catalyst and an appropriate base in an appropriate solvent or solvent mixture at an appropriate temperature and an appropriate amount of time to give intermediates I-3 and I-3a. In some embodiments, the appropriate catalyst is copper acetate. In some embodiments, the appropriate base is pyridine. In some embodiments, the appropriate solvent is dichloromethane. In some embodiments, the appropriate temperature is room temperature and the appropriate amount of time stirred is about 15 hours (overnight).

In some embodiments, an appropriate protecting group is a methyl protecting group. In some embodiments, appropriate conditions to remove a methyl protecting group include using an appropriate reagent in an appropriate solvent at an appropriate temperature and an appropriate amount of time. In some embodiments, the appropriate reagent is boron tribromide. In some embodiments, the appropriate solvent is a chlorinated solvent such as dichloromethane. In some embodiments, a suitable temperature is 0° C. to room temperature and the appropriate amount of time is 15 hours (overnight).

In some embodiments, an appropriate protecting group is a benzyl protecting group. In some embodiments, appropriate conditions to remove a benzyl protecting group include using appropriate hydrogenation conditions using an appropriate catalyst in an appropriate solvent at an appropriate temperature and for an appropriate amount of time. In some embodiments, the appropriate catalyst is palladium on carbon. In some embodiments, the appropriate solvent is THF. In some embodiments, the appropriate temperature is room temperature and the appropriate amount of time stirred under a hydrogen atmosphere at the appropriate pressure is about 2 hours. In some embodiments, the appropriate pressure of hydrogen is atmospheric pressure.

In some embodiments, an appropriate protecting group is a MOM protecting group. In some embodiments, appropriate conditions to remove a MOM protecting group include using a suitable acid in a suitable solvent or solvent mixture at an appropriate temperature and an appropriate amount of time. In some embodiments, the appropriate acid is hydrochloric acid. In some embodiments, the appropriate solvent mixture is THF:methanol. In some embodiments, the suitable temperature is 90° C. and the appropriate amount of time is about 30 min.

In some embodiments, an appropriate protecting group is a TBS protecting group. In some embodiments, appropriate conditions to remove a TBS protecting group include using an appropriate reagent in an appropriate solvent at an appropriate temperature and amount of time. In some embodiments, the appropriate reagent is ammonium fluoride. In some embodiments, the appropriate solvent is methanol. In some embodiments, the appropriate temperature is 80° C. and the appropriate amount of time is 1 hour.

In some embodiments, the intermediate I-2 is reacted with an appropriate phenol to directly give compound I-4.

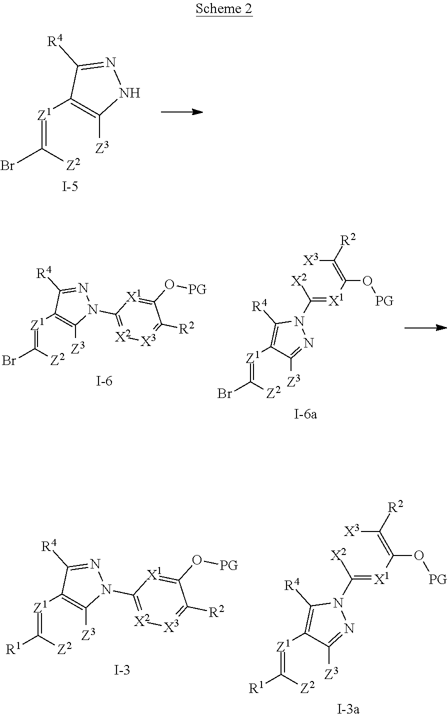

In some embodiments, compounds described herein are prepared as outlined in Scheme 2.

In some embodiments, intermediate I-5 is reacted with an appropriate boronic acid or an appropriate boronic ester under appropriate Chan-Lam coupling reaction conditions using an appropriate catalyst and an appropriate base in an appropriate solvent or solvent mixture at an appropriate temperature and an appropriate amount of time to give intermediates I-6 and I-6a. In some embodiments, the appropriate catalyst is copper acetate. In some embodiments, the appropriate base is pyridine. In some embodiments, the appropriate solvent is dichloromethane. In some embodiments, the appropriate temperature is room temperature and the appropriate amount of time stirred is overnight.

In some embodiments, intermediates I-6 and I-6a are reacted under appropriate Suzuki coupling reaction conditions to provide intermediates I-3 and I-3a. In some embodiments, appropriate Suzuki conditions include using an appropriate catalyst and boronic acid or boronic ester with an appropriate base and solvent at an appropriate time and at an appropriate temperature. In some embodiments, the appropriate catalyst is tetrakis(triphenylphosphine)palladium(0). In some embodiments, the appropriate base is sodium carbonate. In some embodiments, the appropriate solvent mixture is dioxane:water. In some embodiments, the suitable temperature is 90° C. and the appropriate amount of time stirred is about 100 minutes.

In some embodiments, the phenol protection group of intermediate I-6 is removed prior to Suzuki coupling to provide compound I-4.

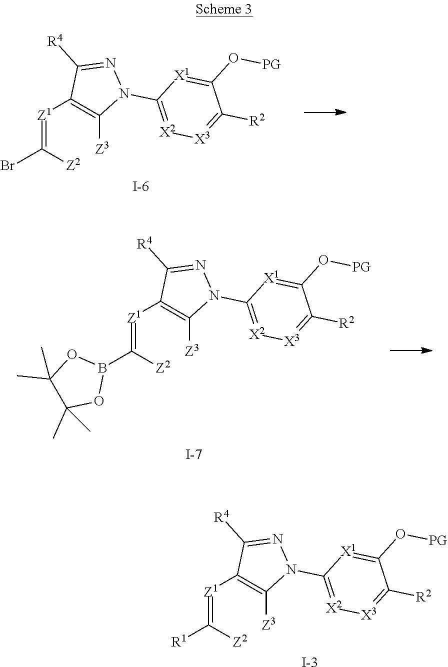

In some embodiments, compounds described herein are prepared as outlined in Scheme 3.

In some embodiments, intermediate I-6 is reacted with bis(pinacolato)diboron using an appropriate catalyst and an appropriate base in an appropriate solvent or solvent mixture at an appropriate temperature and an appropriate amount of time to give intermediate I-7. In some embodiments, the appropriate catalyst is 1,1′-bis(diphenylphosphino)ferrocene dichloropalladium (II). In some embodiments, the appropriate base is potassium acetate. In some embodiments, the appropriate solvent is toluene. In some embodiments, the appropriate temperature is 90° C. and the appropriate amount of time stirred is overnight.

In some embodiments, intermediate I-7 is reacted under appropriate Suzuki coupling reaction conditions to provide compound I-3. In some embodiments, appropriate Suzuki conditions include using an appropriate catalyst and boronic acid or boronic ester with an appropriate base and solvent at an appropriate time and at an appropriate temperature. In some embodiments, the appropriate catalyst is tetrakis(triphenylphosphine)palladium(0). In some embodiments, the appropriate base is sodium carbonate. In some embodiments, the appropriate solvent mixture is dioxane:water. In some embodiments, the appropriate temperature is 80° C. and the appropriate amount of time stirred is about 100 minutes.

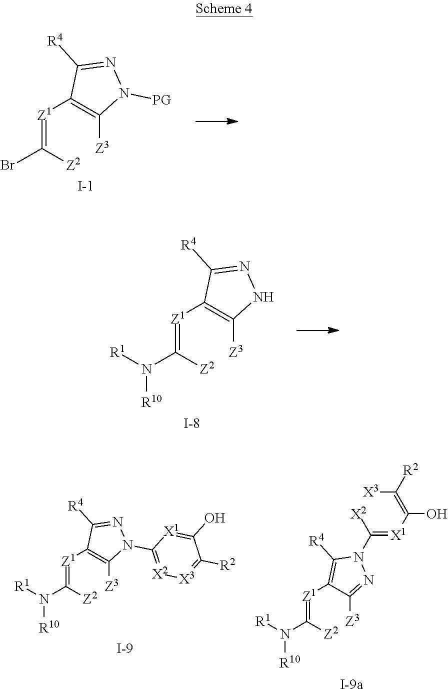

In some embodiments, compounds described herein are prepared as outlined in Scheme 4.

In some embodiments, intermediate I-1 is reacted with an appropriate amine under appropriate Buchwald coupling reaction conditions followed by removal of an appropriate protecting group to provide compound I-8. In some embodiments, appropriate Buchwald conditions include using an appropriate catalyst with an appropriate base and appropriate solvent at an appropriate time and at an appropriate temperature. In some embodiments, the appropriate catalyst is tris(dibenzylideneacetone)dipalladium (0). In some embodiments, the appropriate catalyst ligand is RuPhos. In some embodiments, the appropriate base is sodium tert-butoxide. In some embodiments, the appropriate solvent is toluene. In some embodiments, the appropriate temperature is 100° C. and the appropriate amount of time stirred is about 30 minutes to 2 days.

In some embodiments, the appropriate protecting group is a tetrahydropyran protecting group. In some embodiments, appropriate conditions to remove a tetrahydropyran protecting group include using an appropriate reagent in an appropriate solvent at an appropriate temperature and an appropriate amount of time. In some embodiments, the appropriate reagent is trifluoroacetic acid. In some embodiments, the appropriate solvent is a chlorinated solvent such as dichloromethane. In some embodiments, the appropriate temperature is room temperature and the appropriate amount of time is about 15 hours (overnight).

In some embodiments, intermediate I-8 is reacted with an appropriate aryl-halide under appropriate Ullmann-type coupling conditions using an appropriate catalyst and catalyst ligand and an appropriate base in an appropriate solvent at an appropriate temperature and an appropriate amount of time to give I-9 and I-9a. In some embodiments, a suitable aryl-halide is an aryl-bromide. In some embodiments, the appropriate catalyst is copper iodide. In some embodiments, the appropriate catalyst ligand is trans-N,N-dimethylcyclohexane-1,2-diamine. In some embodiments, the appropriate base is potassium phosphate. In some embodiments, the appropriate solvent is DMSO. In some embodiments, the suitable temperature is 100° C. and the appropriate amount of time stirred is overnight to 2 days.

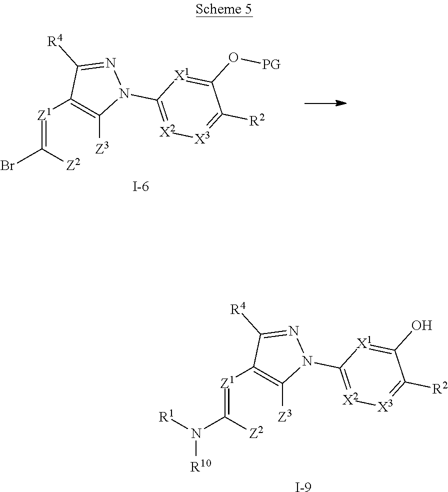

In some embodiments, compounds described herein are prepared as outlined in Scheme 5.

In some embodiments, intermediate I-6 is reacted with an appropriate amine under appropriate Buchwald coupling reaction conditions followed by removal of an appropriate phenol protecting group to provide I-9. In some embodiments, appropriate Buchwald conditions include using an appropriate catalyst with an appropriate base and solvent at an appropriate time and at an appropriate temperature. In some embodiments, the appropriate catalyst is tris(dibenzylideneacetone)dipalladium (0). In some embodiments, the appropriate catalyst ligand is RuPhos. In some embodiments, the appropriate base is sodium tert-butoxide. In some embodiments, the appropriate solvent is toluene or dioxane. In some embodiments, the appropriate temperature is 100° C. and the appropriate amount of time is about 90 minutes to 15 hours (overnight).

In some embodiments, an appropriate protecting group is a MOM protecting group. In some embodiments, appropriate conditions to remove a MOM protecting group include using a suitable acid in a suitable solvent or solvent mixture at an appropriate temperature and an appropriate amount of time. In some embodiments, the appropriate acid is hydrochloric acid. In some embodiments, the appropriate solvent mixture is THF:methanol. In some embodiments, the suitable temperature is 50° C. and the appropriate amount of time is about 15 hours (overnight).

In some embodiments, an appropriate protecting group is a methyl protecting group. In some embodiments, appropriate conditions to remove a methyl protecting group include using an appropriate reagent in an appropriate solvent at an appropriate temperature and an appropriate amount of time. In some embodiments, the appropriate reagent is boron tribromide. In some embodiments, the appropriate solvent is a chlorinated solvent such as dichloromethane. In some embodiments, the appropriate temperature is −78° C. to room temperature and the appropriate amount of time is about 15 hours (overnight).

In some embodiments, an appropriate protecting group is a benzyl protecting group. In some embodiments, appropriate conditions to remove a benzyl protecting group include using appropriate hydrogenation conditions using an appropriate catalyst in an appropriate solvent at an appropriate temperature and an appropriate amount of time. In some embodiments, the appropriate catalyst is palladium on carbon. In some embodiments, the appropriate solvent is THF. In some embodiments, the appropriate temperature is room temperature and the appropriate amount of time stirred under a hydrogen atmosphere at an appropriate pressure is about 1 hour. In some embodiments, the appropriate pressure of hydrogen is atmospheric pressure.

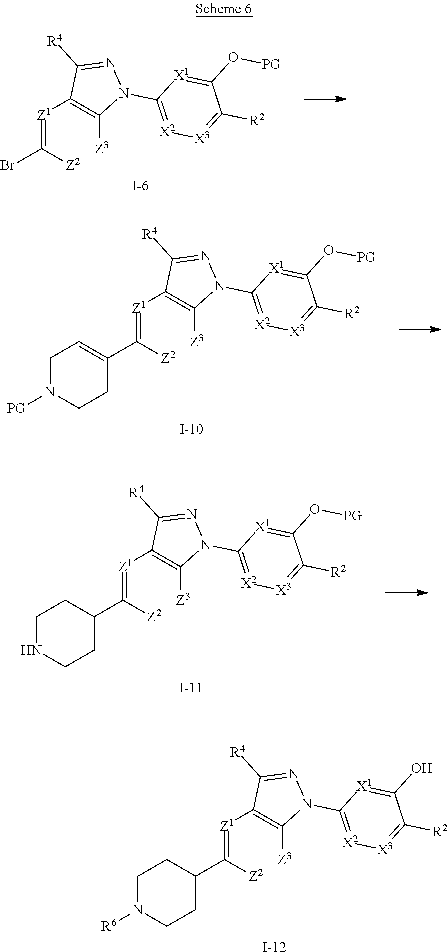

In some embodiments, compounds described herein are prepared as outlined in Scheme 6.

In some embodiments, intermediate I-6 is reacted with an appropriate boronic acid or ester under appropriate Suzuki coupling reaction to provide intermediate I-10. In some embodiments, appropriate Suzuki conditions include using an appropriate catalyst with an appropriate base and an appropriate solvent or solvent mixture at an appropriate time and at an appropriate temperature. In some embodiments, the appropriate catalyst is tetrakis(triphenylphosphine)palladium(0). In some embodiments, the appropriate base is sodium carbonate. In some embodiments, the appropriate solvent mixture is dioxane:water. In some embodiments, the appropriate temperature is 90° C. and the appropriate amount of time stirred is about 2.5 hours.

In some embodiments, intermediate I-10 is reduced under appropriate hydrogenation conditions followed by removal of an appropriate protecting group to provide compound I-11. In some embodiments, appropriate hydrogenation conditions include using an appropriate catalyst with an appropriate solvent at an appropriate time and at an appropriate temperature. In some embodiments, the appropriate catalyst is palladium on carbon. In some embodiments, the appropriate solvent is methanol. In some embodiments, the appropriate temperature is room temperature and the appropriate amount of time stirred under a hydrogen atmosphere at an appropriate pressure is about 2 hours. In some embodiments, the suitable pressure of hydrogen is 15 psi.

In some embodiments, an appropriate protecting group is a Boc-protecting group. In some embodiments, appropriate conditions to remove a Boc protecting group include using a suitable acid in a suitable solvent at an appropriate temperature and amount of time. In some embodiments, the appropriate acid is hydrochloric acid. In some embodiments, the appropriate solvent is methanol. In some embodiments, the appropriate temperature is room temperature and the appropriate amount of time stirred is about 2 hours.

In some embodiments, intermediate I-11 is reacted with an appropriate halide under appropriate sulfonylation conditions followed by removal of an appropriate protecting group to provide compound I-12. In some embodiments, appropriate sulfonylation conditions include using an appropriate reagent and appropriate base with an appropriate solvent at an appropriate time and at an appropriate temperature. In some embodiments, the appropriate reagent is methanesulfonyl chloride. In some embodiments, the appropriate base is pyridine. In some embodiments, the appropriate solvent is a chlorinated solvent such as dichloromethane. In some embodiments, the suitable temperature is room temperature and the appropriate amount of time stirred is about 2 hours.

In some embodiments, an appropriate protecting group is a MOM protecting group. In some embodiments, appropriate conditions to remove a MOM protecting group include using an appropriate acid in an appropriate solvent or solvent mixture at an appropriate temperature and an appropriate amount of time. In some embodiments, the appropriate acid is hydrochloric acid. In some embodiments, the appropriate solvent mixture is THF:methanol. In some embodiments, the appropriate temperature is 90° C. and the appropriate amount of time is about 30 min.

In some embodiments, compounds are prepared as described in the Examples.

### Certain Terminology

Unless otherwise stated, the following terms used in this application have the definitions given below. The use of the term “including” as well as other forms, such as “include”, “includes,” and “included,” is not limiting. The section headings used herein are for organizational purposes only and are not to be construed as limiting the subject matter described.

As used herein, C₁-Cₓ includes C₁-C₂, C₁-C₃ . . . C₁-Cₓ. By way of example only, a group designated as “C₁-C₄” indicates that there are one to four carbon atoms in the moiety, i.e. groups containing 1 carbon atom, 2 carbon atoms, 3 carbon atoms or 4 carbon atoms. Thus, by way of example only, “C₁-C₄ alkyl” indicates that there are one to four carbon atoms in the alkyl group, i.e., the alkyl group is selected from among methyl, ethyl, propyl, iso-propyl, n-butyl, iso-butyl, sec-butyl, and t-butyl.

An “alkyl” group refers to an aliphatic hydrocarbon group. The alkyl group is branched or straight chain. In some embodiments, the “alkyl” group has 1 to 10 carbon atoms, i.e. a C₁-C₁₀alkyl. Whenever it appears herein, a numerical range such as “1 to 10” refers to each integer in the given range; e.g., “1 to 10 carbon atoms” means that the alkyl group consist of 1 carbon atom, 2 carbon atoms, 3 carbon atoms, 4 carbon atoms, 5 carbon atoms, 6 carbon atoms, etc., up to and including 10 carbon atoms, although the present definition also covers the occurrence of the term “alkyl” where no numerical range is designated. In some embodiments, an alkyl is a C₁-C₆alkyl. In one aspect the alkyl is methyl, ethyl, propyl, iso-propyl, n-butyl, iso-butyl, sec-butyl, or t-butyl. Typical alkyl groups include, but are in no way limited to, methyl, ethyl, propyl, isopropyl, butyl, isobutyl, sec-butyl, tertiary butyl, pentyl, neopentyl, or hexyl.

An “alkylene” group refers to a divalent alkyl group. Any of the above mentioned monovalent alkyl groups may be an alkylene by abstraction of a second hydrogen atom from the alkyl. In some embodiments, an alkylene is a C₁-C₆alkylene. In other embodiments, an alkylene is a C₁-C₄alkylene. In certain embodiments, an alkylene comprises one to four carbon atoms (e.g., C₁-C₄ alkylene). In other embodiments, an alkylene comprises one to three carbon atoms (e.g., C₁-C₃ alkylene). In other embodiments, an alkylene comprises one to two carbon atoms (e.g., C₁-C₂ alkylene). In other embodiments, an alkylene comprises one carbon atom (e.g., C₁ alkylene). In other embodiments, an alkylene comprises two carbon atoms (e.g., C₂ alkylene). In other embodiments, an alkylene comprises two to four carbon atoms (e.g., C₂-C₄ alkylene). Typical alkylene groups include, but are not limited to, —CH₂—, —CH(CH₃)—, —C(CH₃)₂—, —CH₂CH₂—, —CH₂CH(CH₃)—, —CH₂C(CH₃)₂—, —CH₂CH₂CH₂—, —CH₂CH₂CH₂CH₂—, and the like.

“Deuteroalkyl” refers to an alkyl group where 1 or more hydrogen atoms of an alkyl are replaced with deuterium.

The term “alkenyl” refers to a type of alkyl group in which at least one carbon-carbon double bond is present. In one embodiment, an alkenyl group has the formula —C(R)═CR₂, wherein R refers to the remaining portions of the alkenyl group, which may be the same or different. In some embodiments, R is H or an alkyl. In some embodiments, an alkenyl is selected from ethenyl (i.e., vinyl), propenyl (i.e., allyl), butenyl, pentenyl, pentadienyl, and the like. Non-limiting examples of an alkenyl group include —CH═CH₂, —C(CH₃)═CH₂, —CH═CHCH₃, —C(CH₃)═CHCH₃, and —CH₂CH═CH₂.

The term “alkynyl” refers to a type of alkyl group in which at least one carbon-carbon triple bond is present. In one embodiment, an alkenyl group has the formula —C≡C—R, wherein R refers to the remaining portions of the alkynyl group. In some embodiments, R is H or an alkyl. In some embodiments, an alkynyl is selected from ethynyl, propynyl, butynyl, pentynyl, hexynyl, and the like. Non-limiting examples of an alkynyl group include —C≡CH, —C≡CCH₃—C≡CCH₂CH₃, —CH₂C≡CH.

An “alkoxy” group refers to a (alkyl)O— group, where alkyl is as defined herein.

The term “alkylamine” refers to the —N(alkyl)ₓH_{y} group, where x is 0 and y is 2, or where x is 1 and y is 1, or where x is 2 and y is 0.

The term “aromatic” refers to a planar ring having a delocalized π-electron system containing 4n+2π electrons, where n is an integer. The term “aromatic” includes both carbocyclic aryl (“aryl”, e.g., phenyl) and heterocyclic aryl (or “heteroaryl” or “heteroaromatic”) groups (e.g., pyridine). The term includes monocyclic or fused-ring polycyclic (i.e., rings which share adjacent pairs of carbon or nitrogen atoms) groups.

The term “carbocyclic” or “carbocycle” refers to a ring or ring system where the atoms forming the backbone of the ring are all carbon atoms. The term thus distinguishes carbocyclic from “heterocyclic” rings or “heterocycles” in which the ring backbone contains at least one atom which is different from carbon. In some embodiments, at least one of the two rings of a bicyclic carbocycle is aromatic. In some embodiments, both rings of a bicyclic carbocycle are aromatic. Carbocycle includes cycloalkyl and aryl.

As used herein, the term “aryl” refers to an aromatic ring wherein each of the atoms forming the ring is a carbon atom. In one aspect, aryl is phenyl or a naphthyl. In some embodiments, an aryl is a phenyl. In some embodiments, an aryl is a C₆-C₁₀aryl. Depending on the structure, an aryl group is a monoradical or a diradical (i.e., an arylene group).

The term “cycloalkyl” refers to a monocyclic or polycyclic aliphatic, non-aromatic group, wherein each of the atoms forming the ring (i.e. skeletal atoms) is a carbon atom. In some embodiments, cycloalkyls are spirocyclic or bridged compounds. In some embodiments, cycloalkyls are fully saturated. In some embodiments, cycloalkyls are partially unsaturated. In some embodiments, cycloalkyls are optionally fused with an aromatic ring, and the point of attachment is at a carbon that is not an aromatic ring carbon atom. Cycloalkyl groups include groups having from 3 to 10 ring atoms. In some embodiments, cycloalkyl groups are selected from among cyclopropyl, cyclobutyl, cyclopentyl, cyclopentenyl, cyclohexyl, cyclohexenyl, cycloheptyl, cyclooctyl, spiro[2.2]pentyl, norbornyl and bicyclo[1.1.1]pentyl. In some embodiments, a cycloalkyl is a C₃-C₆cycloalkyl. In some embodiments, a cycloalkyl is a monocyclic cycloalkyl. Monocyclic cycloalkyls include, but are not limited to, cyclopropyl, cyclobutyl, cyclopentyl, cyclohexyl, cycloheptyl, and cyclooctyl. Poly cyclic cycloalkyls include, for example, adamantyl, norbornyl (i.e., bicyclo[2.2.1]heptanyl), norbornenyl, decalinyl, 7,7-dimethyl-bicyclo[2.2.1]heptanyl, and the like

The term “halo” or, alternatively, “halogen” or “halide” means fluoro, chloro, bromo or iodo. In some embodiments, halo is fluoro, chloro, or bromo.

The term “haloalkyl” refers to an alkyl in which one or more hydrogen atoms are replaced by a halogen atom. In one aspect, a fluoroalkyl is a C₁-C₆fluoroalkyl.

The term “fluoroalkyl” refers to an alkyl in which one or more hydrogen atoms are replaced by a fluorine atom. In one aspect, a fluoroalkyl is a C₁-C₆fluoroalkyl. In some embodiments, a fluoroalkyl is selected from trifluoromethyl, difluoromethyl, fluoromethyl, 2,2,2-trifluoroethyl, 1-fluoromethyl-2-fluoroethyl, and the like.

The term “heteroalkyl” refers to an alkyl group in which one or more skeletal atoms of the alkyl are selected from an atom other than carbon, e.g., oxygen, nitrogen (e.g., —NH—, —N(alkyl)-, sulfur, or combinations thereof. A heteroalkyl is attached to the rest of the molecule at a carbon atom of the heteroalkyl. In one aspect, a heteroalkyl is a C₁-C₆heteroalkyl.

The term “heteroalkylene” refers to a divalent heteroalkyl group.

The term “heterocycle” or “heterocyclic” refers to heteroaromatic rings (also known as heteroaryls) and heterocycloalkyl rings (also known as heteroalicyclic groups) containing one to four heteroatoms in the ring(s), where each heteroatom in the ring(s) is selected from O, S and N, wherein each heterocyclic group has from 3 to 10 atoms in its ring system, and with the proviso that any ring does not contain two adjacent O or S atoms. In some embodiments, heterocycles are monocyclic, bicyclic, polycyclic, spirocyclic or bridged compounds. Non-aromatic heterocyclic groups (also known as heterocycloalkyls) include rings having 3 to 10 atoms in its ring system and aromatic heterocyclic groups include rings having 5 to 10 atoms in its ring system. The heterocyclic groups include benzo-fused ring systems. Examples of non-aromatic heterocyclic groups are pyrrolidinyl, tetrahydrofuranyl, dihydrofuranyl, tetrahydrothienyl, oxazolidinonyl, tetrahydropyranyl, dihydropyranyl, tetrahydrothiopyranyl, piperidinyl, morpholinyl, thiomorpholinyl, thioxanyl, piperazinyl, aziridinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, pyrrolin-2-yl, pyrrolin-3-yl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, 3H-indolyl, indolin-2-onyl, isoindolin-1-onyl, isoindoline-1,3-dionyl, 3,4-dihydroisoquinolin-1(2H)-onyl, 3,4-dihydroquinolin-2(1H)-onyl, isoindoline-1,3-dithionyl, benzo[d]oxazol-2(3H)-onyl, 1H-benzo[d]imidazol-2(3H)-onyl, benzo[d]thiazol-2(3H)-onyl, and quinolizinyl. Examples of aromatic heterocyclic groups are pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl. The foregoing groups are either C-attached (or C-linked) or N-attached where such is possible. For instance, a group derived from pyrrole includes both pyrrol-1-yl (N-attached) or pyrrol-3-yl (C-attached). Further, a group derived from imidazole includes imidazol-1-yl or imidazol-3-yl (both N-attached) or imidazol-2-yl, imidazol-4-yl or imidazol-5-yl (all C-attached). The heterocyclic groups include benzo-fused ring systems. Non-aromatic heterocycles are optionally substituted with one or two oxo (═O) moieties, such as pyrrolidin-2-one. In some embodiments, at least one of the two rings of a bicyclic heterocycle is aromatic. In some embodiments, both rings of a bicyclic heterocycle are aromatic.

The terms “heteroaryl” or, alternatively, “heteroaromatic” refers to an aryl group that includes one or more ring heteroatoms selected from nitrogen, oxygen and sulfur. Illustrative examples of heteroaryl groups include monocyclic heteroaryls and bicyclic heteroaryls. Monocyclic heteroaryls include pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, pyridazinyl, triazinyl, oxadiazolyl, thiadiazolyl, and furazanyl. Bicyclic heteroaryls include indolizine, indole, benzofuran, benzothiophene, indazole, benzimidazole, benzotriazole, purine, quinolizine, quinoline, isoquinoline, cinnoline, phthalazine, quinazoline, quinoxaline, 1,8-naphthyridine, and pteridine. In some embodiments, a heteroaryl contains 0-4N atoms in the ring. In some embodiments, a heteroaryl contains 1-4 N atoms in the ring. In some embodiments, a heteroaryl contains 0-4 N atoms, 0-10 atoms, and 0-1 S atoms in the ring. In some embodiments, a heteroaryl contains 1-4 N atoms, 0-10 atoms, and 0-1 S atoms in the ring. In some embodiments, heteroaryl is a C₁-C₉heteroaryl. In some embodiments, monocyclic heteroaryl is a C₁-C₅heteroaryl. In some embodiments, monocyclic heteroaryl is a 5-membered or 6-membered heteroaryl. In some embodiments, bicyclic heteroaryl is a C₆-C₉heteroaryl.

A “heterocycloalkyl” or “heteroalicyclic” group refers to a cycloalkyl group that includes at least one heteroatom selected from nitrogen, oxygen and sulfur. In some embodiments, heterocycloalkyls are spirocyclic or bridged compounds. In some embodiments, heterocycloalkyls are fully saturated. In some embodiments, heterocycloalkyls are partially unsaturated. In some embodiments, a heterocycloalkyl is fused with an aryl or heteroaryl. In some embodiments, the heterocycloalkyl is oxazolidinonyl, pyrrolidinyl, tetrahydrofuranyl, tetrahydrothienyl, tetrahydropyranyl, tetrahydrothiopyranyl, piperidinyl, morpholinyl, thiomorpholinyl, piperazinyl, piperidin-2-onyl, pyrrolidine-2,5-dithionyl, pyrrolidine-2,5-dionyl, pyrrolidinonyl, imidazolidinyl, imidazolidin-2-onyl, or thiazolidin-2-onyl. The term heteroalicyclic also includes all ring forms of the carbohydrates, including but not limited to the monosaccharides, the disaccharides and the oligosaccharides. In one aspect, a heterocycloalkyl is a C₂-C₁₀heterocycloalkyl. In another aspect, a heterocycloalkyl is a C₄-C₁₀heterocycloalkyl. In some embodiments, a heterocycloalkyl contains 0-2N atoms in the ring. In some embodiments, a heterocycloalkyl contains 0-2N atoms, 0-2 O atoms and 0-1 S atoms in the ring.

The term “bond” or “single bond” refers to a chemical bond between two atoms, or two moieties when the atoms joined by the bond are considered to be part oflarger substructure. In one aspect, when a group described herein is a bond, the referenced group is absent thereby allowing a bond to be formed between the remaining identified groups.

The term “moiety” refers to a specific segment or functional group of a molecule. Chemical moieties are often recognized chemical entities embedded in or appended to a molecule.

The term “optionally substituted” or “substituted” means that the referenced group is optionally substituted with one or more additional group(s). In some other embodiments, optional substituents are individually and independently selected from D, halogen, —CN, —NH₂, —NH(alkyl), —N(alkyl)₂, —OH, —CO₂H, —CO₂alkyl, —C(═O)NH₂, —C(═O)NH(alkyl), —C(═O)N(alkyl)₂, —S(═O)₂NH₂, —S(═O)₂NH(alkyl), —S(═O)₂N(alkyl)₂, —CH₂CO₂H, —CH₂CO₂alkyl, —CH₂C(═O)NH₂, —CH₂C(═O)NH(alkyl), —CH₂C(═O)N(alkyl)₂, —CH₂S(═O)₂NH₂, —CH₂S(═O)₂NH(alkyl), —CH₂S(═O)₂N(alkyl)₂, alkyl, alkenyl, alkynyl, cycloalkyl, fluoroalkyl, heteroalkyl, alkoxy, fluoroalkoxy, heterocycloalkyl, aryl, heteroaryl, aryloxy, alkylthio, arylthio, alkylsulfoxide, arylsulfoxide, alkylsulfone, and arylsulfone. The term “optionally substituted” or “substituted” means thatthe referenced group is optionally substituted with one or more additional group(s) individually and independently selected from D, halogen, —CN, —NH₂, —NH(alkyl), —N(alkyl)₂, —OH, —CO₂H, —CO₂alkyl, —C(═O)NH₂, —C(═O)NH(alkyl), —C(═O)N(alkyl)₂, —S(═O)₂NH₂, —S(═O)₂NH(alkyl), —S(═O)₂N(alkyl)₂, alkyl, cycloalkyl, fluoroalkyl, heteroalkyl, alkoxy, fluoroalkoxy, heterocycloalkyl, aryl, heteroaryl, aryloxy, alkylthio, arylthio, alkylsulfoxide, arylsulfoxide, alkylsulfone, and arylsulfone. In some other embodiments, optional substituents are independently selected from D, halogen, —CN, —NH₂, —NH(CH₃), —N(CH₃)₂, —OH, —CO₂H, —CO₂(C₁-C₄alkyl), —C(═O)NH₂, —C(═O)NH(C₁-C₄alkyl), —C(═O)N(C₁-C₄alkyl)₂, —S(═O)₂NH₂, —S(═O)₂NH(C₁-C₄alkyl), —S(═O)₂N(C₁-C₄alkyl)₂, C₁-C₄alkyl, C₃-C₆cycloalkyl, C₁-C₄fluoroalkyl, C₁-C₄heteroalkyl, C₁-C₄alkoxy, C₁-C₄fluoroalkoxy, —SC₁-C₄alkyl, —S(═O)C₁-C₄alkyl, and —S(═O)₂C₁-C₄alkyl. In some embodiments, optional substituents are independently selected from D, halogen, —CN, —NH₂, —OH, —NH(CH₃), —N(CH₃)₂, —CH₃, —CH₂CH₃, —CF₃, —OCH₃, and —OCF₃. In some embodiments, substituted groups are substituted with one or two of the preceding groups. In some embodiments, substituted groups are substituted with one of the preceding groups. In some embodiments, an optional substituent on an aliphatic carbon atom (acyclic or cyclic) includes oxo (═O).

The term “acceptable” with respect to a formulation, composition or ingredient, as used herein, means having no persistent detrimental effect on the general health of the subject being treated.

The term “modulate” as used herein, means to interact with a target either directly or indirectly so as to alter the activity of the target, including, by way of example only, to enhance the activity of the target, to inhibit the activity of the target, to limit the activity of the target, or to extend the activity of the target.

The term “modulator” as used herein, refers to a molecule that interacts with a target either directly or indirectly. The interactions include, but are not limited to, the interactions of an agonist, partial agonist, an inverse agonist, antagonist, degrader, or combinations thereof. In some embodiments, a modulator is an agonist.

The terms “administer,” “administering”, “administration,” and the like, as used herein, refer to the methods that may be used to enable delivery of compounds or compositions to the desired site of biological action. These methods include, but are not limited to oral routes, intraduodenal routes, parenteral injection (including intravenous, subcutaneous, intraperitoneal, intramuscular, intravascular or infusion), topical and rectal administration. Those of skill in the art are familiar with administration techniques that can be employed with the compounds and methods described herein. In some embodiments, the compounds and compositions described herein are administered orally.

The terms “co-administration” or the like, as used herein, are meant to encompass administration of the selected therapeutic agents to a single patient, and are intended to include treatment regimens in which the agents are administered by the same or different route of administration or at the same or different time.

The terms “effective amount” or “therapeutically effective amount,” as used herein, refer to a sufficient amount of an agent or a compound being administered, which will relieve to some extent one or more of the symptoms of the disease or condition being treated. The result includes reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. An appropriate “effective” amount in any individual case is optionally determined using techniques, such as a dose escalation study.

The terms “enhance” or “enhancing,” as used herein, means to increase or prolong either in potency or duration a desired effect. Thus, in regard to enhancing the effect of therapeutic agents, the term “enhancing” refers to the ability to increase or prolong, either in potency or duration, the effect of other therapeutic agents on a system. An “enhancing-effective amount,” as used herein, refers to an amount adequate to enhance the effect of another therapeutic agent in a desired system.

The terms “kit” and “article of manufacture” are used as synonyms.

The term “subject” or “patient” encompasses mammals. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. In one aspect, the mammal is a human.

The terms “treat,” “treating” or “treatment,” as used herein, include alleviating, abating or ameliorating at least one symptom of a disease or condition, preventing additional symptoms, inhibiting the disease or condition, e.g., arresting the development of the disease or condition, relieving the disease or condition, causing regression of the disease or condition, relieving a condition caused by the disease or condition, or stopping the symptoms of the disease or condition either prophylactically and/or therapeutically.

### Pharmaceutical Compositions

In some embodiments, the compounds described herein are formulated into pharmaceutical compositions. Pharmaceutical compositions are formulated in a conventional manner using one or more pharmaceutically acceptable inactive ingredients that facilitate processing of the active compounds into preparations that are used pharmaceutically. Proper formulation is dependent upon the route of administration chosen. A summary of pharmaceutical compositions described herein is found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), herein incorporated by reference for such disclosure.

In some embodiments, the compounds described herein are administered either alone or in combination with pharmaceutically acceptable carriers, excipients or diluents, in a pharmaceutical composition. Administration of the compounds and compositions described herein can be affected by any method that enables delivery of the compounds to the site of action. These methods include, though are not limited to delivery via enteral routes (including oral, gastric or duodenal feeding tube, rectal suppository and rectal enema), parenteral routes (injection or infusion, including intraarterial, intracardiac, intradermal, intraduodenal, intramedullary, intramuscular, intraosseous, intraperitoneal, intrathecal, intravascular, intravenous, intravitreal, epidural and subcutaneous), inhalational, transdermal, transmucosal, sublingual, buccal and topical (including epicutaneous, dermal, enema, eye drops, ear drops, intranasal, vaginal) administration, although the most suitable route may depend upon for example the condition and disorder ofthe recipient. By way of example only, compounds described herein can be administered locally to the area in need of treatment, by for example, local infusion during surgery, topical application such as creams or ointments, injection, catheter, or implant. The administration can also be by direct injection at the site of a diseased tissue or organ.

In some embodiments, pharmaceutical compositions suitable for oral administration are presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion. In some embodiments, the active ingredient is presented as a bolus, electuary or paste.

Pharmaceutical compositions which can be used orally include tablets, push-fit capsules made of gelatin, as well as soft, sealed capsules made of gelatin and a plasticizer, such as glycerol or sorbitol. Tablets may be made by compression or molding, optionally with one or more accessory ingredients. Compressed tablets may be prepared by compressing in a suitable machine the active ingredient in a free-flowing form such as a powder or granules, optionally mixed with binders, inert diluents, or lubricating, surface active or dispersing agents. Molded tablets may be made by molding in a suitable machine a mixture of the powdered compound moistened with an inert liquid diluent. In some embodiments, the tablets are coated or scored and are formulated so as to provide slow or controlled release of the active ingredient therein. All formulations for oral administration should be in dosages suitable for such administration. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In some embodiments, stabilizers are added. Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or Dragee coatings for identification or to characterize different combinations of active compound doses.

In some embodiments, pharmaceutical compositions are formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. The compositions may be presented in unit-dose or multi-dose containers, for example sealed ampoules and vials, and may be stored in powder form or in a freeze-dried (lyophilized) condition requiring only the addition of the sterile liquid carrier, for example, saline or sterile pyrogen-free water, immediately prior to use. Extemporaneous injection solutions and suspensions may be prepared from sterile powders, granules and tablets of the kind previously described.

Pharmaceutical compositions for parenteral administration include aqueous and non-aqueous (oily) sterile injection solutions of the active compounds which may contain antioxidants, buffers, bacteriostats and solutes which render the formulation isotonic with the blood of the intended recipient; and aqueous and non-aqueous sterile suspensions which may include suspending agents and thickening agents. Suitable lipophilic solvents or vehicles include fatty oils such as sesame oil, or synthetic fatty acid esters, such as ethyl oleate or triglycerides, orliposomes. Aqueous injection suspensions may contain substanceswhich increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Pharmaceutical compositions may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example, as an emulsion in an acceptable oil) or ion exchange resins, or as sparingly soluble derivatives, for example, as a sparingly soluble salt.

For buccal or sublingual administration, the compositions may take the form of tablets, lozenges, pastilles, or gels formulated in conventional manner. Such compositions may comprise the active ingredient in a flavored basis such as sucrose and acacia or tragacanth.

Pharmaceutical compositions may also be formulated in rectal compositions such as suppositories or retention enemas, e.g., containing conventional suppository bases such as cocoa butter, polyethylene glycol, or other glycerides.

Pharmaceutical compositions may be administered topically, that is by non-systemic administration. This includes the application of a compound of the present invention externally to the epidermis or the buccal cavity and the instillation of such a compound into the ear, eye and nose, such that the compound does not significantly enter the blood stream. In contrast, systemic administration refers to oral, intravenous, intraperitoneal and intramuscular administration.

Pharmaceutical compositions suitable for topical administration include liquid or semi-liquid preparations suitable for penetration through the skin to the site of inflammation such as gels, liniments, lotions, creams, ointments or pastes, and drops suitable for administration to the eye, ear or nose. The active ingredient may comprise, for topical administration, from 0.001% to 10% w/w, for instance from 1% to 2% by weight of the formulation.

Pharmaceutical compositions for administration by inhalation are conveniently delivered from an insufflator, nebulizer pressurized packs or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Alternatively, for administration by inhalation or insufflation, pharmaceutical preparations may take the form of a dry powder composition, for example a powder mix of the compound and a suitable powder base such as lactose or starch. The powder composition may be presented in unit dosage form, in for example, capsules, cartridges, gelatin or blister packs from which the powder may be administered with the aid of an inhalator or insufflator.

In some embodiments, a compound disclosed herein is formulated to provide a controlled release of the compound. Controlled release refers to the release of the compound described herein from a dosage form in which it is incorporated according to a desired profile over an extended period of time. Controlled release profiles include, for example, sustained release, prolonged release, pulsatile release, and delayed release profiles. In contrast to immediate release compositions, controlled release compositions allow delivery of an agent to a subject over an extended period of time according to a predetermined profile. Such release rates can provide therapeutically effective levels of agent for an extended period of time and thereby provide a longer period of pharmacologic response while minimizing side effects as compared to conventional rapid release dosage forms. Such longer periods of response provide for many inherent benefits that are not achieved with the corresponding short acting, immediate release preparations.

Approaches to deliver the intact therapeutic compound to the particular regions of the gastrointestinal tract (e.g., such as the colon), include:
(i) Coating with polymers: The intact molecule can be delivered to the colon without absorbing at the upper part of the intestine by coating of the drug molecule with the suitable polymers, which degrade only in the colon.
(ii) Coating with pH-sensitive polymers: The majority of enteric and colon targeted delivery systems are based on the coating of tablets or pellets, which are filled into conventional hard gelatin capsules. Most commonly used pH-dependent coating polymers are methacrylic acid copolymers, commonly known as Eudragit® S, more specifically Eudragit® L and Eudragit® S. Eudragit® L100 and S 100 are copolymers of methacrylic acid and methyl methacrylate. Additional pH-dependent coating polymers include cellulose acetate phthalate (CAP), hydroxypropyl methylcellulose phthalate (HPMCP), polyvinyl acetate phthalate (PVAP) and cellulose acetate trimelliate.
(iii) Coating with biodegradable polymers;
(iv) Embedding in matrices;
(v) Embedding in biodegradable matrices and hydrogels;
(vi) Embedding in pH-sensitive matrices;
(vii) Timed release systems;
(viii) Redox-sensitive polymers;
(ix) Bioadhesive systems;
(x) Coating with microparticles;
(xi) Osmotic controlled drug delivery.

Another approach towards colon-targeted drug delivery or controlled-release systems includes embedding the drug in polymer matrices to trap it and release it in the colon. These matrices can be pH-sensitive or biodegradable. Matrix-Based Systems, such as multi-matrix (MMX)-based delayed-release tablets, ensure the drug release in the colon.

Additional pharmaceutical approaches to targeted delivery of therapeutics to particular regions of the gastrointestinal tract are known. Chourasia M K, Jain S K, Pharmaceutical approaches to colon targeted drug delivery systems., J Pharm Sci. 2003 January-April; 6(1):33-66. Patel M, Shah T, Amin A. Therapeutic opportunities in colon-specific drug-delivery systems Crit Rev Ther Drug Carrier Syst. 2007; 24(2):147-202. Kumar P, Mishra B. Colon targeted drug delivery systems-an overview. Curr Drug Deliv. 2008 July; 5(3):186-98. Van den Mooter G. Colon drug delivery. Expert Opin Drug Deliv. 2006 January; 3(1):111-25. Seth Amidon, Jack E. Brown, and Vivek S. Dave, Colon-Targeted Oral Drug Delivery Systems: Design Trends and Approaches, AAPS PharmSciTech. 2015 August; 16(4): 731-741.

It should be understood that in addition to the ingredients particularly mentioned above, the compounds and compositions described herein may include other agents conventional in the art having regard to the type of formulation in question, for example those suitable for oral administration may include flavoring agents.

### Methods of Dosing and Treatment Regimens

In one embodiment, the compounds described herein, or a pharmaceutically acceptable salt thereof, are used in the preparation of medicaments for the treatment of diseases or conditions in a mammal that would benefit from administration of an HSD17B13 inhibitor. Methods for treating any of the diseases or conditions described herein in a mammal in need of such treatment, involves administration of pharmaceutical compositions that include at least one compound described herein or a pharmaceutically acceptable salt, active metabolite, prodrug, or pharmaceutically acceptable solvate thereof, in therapeutically effective amounts to said mammal.

In some embodiments, described herein is a method of treating or preventing a liver disease or condition in a mammal, comprising administering to the mammal a compound of Formula (I′), (I), (Ia′), (Ia), or (II), or a pharmaceutically acceptable salt or solvate thereof. In some embodiments, the liver disease or condition is an alcoholic liver disease or condition. In some embodiments, the liver disease or condition is a nonalcoholic liver disease or condition. In some embodiments, the liver disease or condition is liver inflammation, fatty liver (steatosis), liver fibrosis, hepatitis, cirrhosis, hepatocellular carcinoma, or combinations thereof. In some embodiments, the liver disease or condition is primary biliary cirrhosis, primary sclerosing cholangitis, cholestasis, nonalcoholic steatohepatitis (NASH), nonalcoholic fatty liver disease (NAFLD), or combinations thereof. In some embodiments, the liver disease or condition described herein is a chronic liver disease or condition.

In some embodiments, described herein is a method of modulating HSD17B13 activity in a mammal, comprising administering to the mammal a compound of Formula (I′), (I), (Ia′), (Ia), or (II), or a pharmaceutically acceptable salt or solvate thereof. In some embodiments, modulating comprises inhibiting HSD17B13 activity. In some embodiments of a method of modulating HSD17B13 activity in a mammal, the mammal has a liver disease or condition selected from liver inflammation, fatty liver (steatosis), liver fibrosis, hepatitis, cirrhosis, hepatocellular carcinoma, and combinations thereof. In some embodiments of a method of modulating HSD17B13 activity in a mammal, the mammal has a liver disease or condition selected from primary biliary cirrhosis, primary sclerosing cholangitis, cholestasis, nonalcoholic steatohepatitis (NASH), nonalcoholic fatty liver disease (NAFLD), and combinations thereof.

In certain embodiments, the compositions containing the compound(s) described herein are administered for prophylactic and/or therapeutic treatments. In certain therapeutic applications, the compositions are administered to a patient already suffering from a disease or condition, in an amount sufficient to cure or at least partially arrest at least one of the symptoms of the disease or condition. Amounts effective for this use depend on the severity and course of the disease or condition, previous therapy, the patient's health status, weight, and response to the drugs, and the judgment of the treating physician. Therapeutically effective amounts are optionally determined by methods including, but not limited to, a dose escalation and/or dose ranging clinical trial.

In prophylactic applications, compositions containing the compounds described herein are administered to a patient susceptible to or otherwise at risk of a particular disease, disorder, or condition. Such an amount is defined to be a “prophylactically effective amount or dose.” In this use, the precise amounts also depend on the patient's state of health, weight, and the like. When used in patients, effective amounts for this use will depend on the severity and course of the disease, disorder, or condition, previous therapy, the patient's health status and response to the drugs, and the judgment of the treating physician. In one aspect, prophylactic treatments include administering to a mammal, who previously experienced at least one symptom of the disease being treated and is currently in remission, a pharmaceutical composition comprising a compound described herein, or a pharmaceutically acceptable salt thereof, in order to prevent a return of the symptoms of the disease or condition.

In certain embodiments wherein the patient's condition does not improve, upon the doctor's discretion, the compounds are administered chronically, that is, for an extended period of time, including throughout the duration of the patient's life in order to ameliorate or otherwise control or limit the symptoms of the patient's disease or condition.

In certain embodiments wherein a patient's status does improve, the dose of drug being administered is temporarily reduced or temporarily suspended for a certain length of time (i.e., a “drug holiday”). In specific embodiments, the length of the drug holiday is between 2 days and 1 year, including by way of example only, 2 days, 3 days, 4 days, 5 days, 6 days, 7 days, 10 days, 12 days, 15 days, 20 days, 28 days, or more than 28 days. The dose reduction during a drug holiday is, by way of example only, by 10%-100%, including by way of example only 10%, 15%, 20%, 25%, 30%, 35%, 40%, 45%, 50%, 55%, 60%, 65%, 70%, 75%, 80%, 85%, 90%, 95%, and 100%.

Once improvement of the patient's conditions has occurred, a maintenance dose is administered if necessary. Subsequently, in specific embodiments, the dosage or the frequency of administration, or both, is reduced, as a function of the symptoms, to a level at which the improved disease, disorder, or condition is retained. In certain embodiments, however, the patient requires intermittent treatment on a long-term basis upon any recurrence of symptoms.

The amount of a given agent that corresponds to such an amount varies depending upon factors such as the particular compound, disease condition and its severity, the identity (e.g., weight, sex) of the subject or host in need of treatment, but nevertheless is determined according to the particular circumstances surrounding the case, including, e.g., the specific agent being administered, the route of administration, the condition being treated, and the subject or host being treated.

In general, however, doses employed for adult human treatment are typically in the range of 0.01 mg-5000 mg per day. In one aspect, doses employed for adult human treatment are from about 1 mg to about 1000 mg per day. In one embodiment, the desired dose is conveniently presented in a single dose or in divided doses administered simultaneously or at appropriate intervals, for example as two, three, four or more sub-doses per day.

In one embodiment, the daily dosages appropriate for the compound described herein, or a pharmaceutically acceptable salt thereof, are from about 0.01 to about 50 mg/kg per body weight. In some embodiments, the daily dosage or the amount of active in the dosage form are lower or higher than the ranges indicated herein, based on a number of variables in regard to an individual treatment regime. In various embodiments, the daily and unit dosages are altered depending on a number of variables including, but not limited to, the activity of the compound used, the disease or condition to be treated, the mode of administration, the requirements of the individual subject, the severity of the disease or condition being treated, and the judgment of the practitioner.

Toxicity and therapeutic efficacy of such therapeutic regimens are determined by standard pharmaceutical procedures in cell cultures or experimental animals, including, but not limited to, the determination of the LD₅₀ and the ED₅₀. The dose ratio between the toxic and therapeutic effects is the therapeutic index and it is expressed as the ratio between LD₅₀ and ED₅₀. In certain embodiments, the data obtained from cell culture assays and animal studies are used in formulating the therapeutically effective daily dosage range and/or the therapeutically effective unit dosage amount foruse in mammals, including humans. In some embodiments, the daily dosage amount of the compounds described herein lies within a range of circulating concentrations that include the ED₅₀ with minimal toxicity. In certain embodiments, the daily dosage range and/or the unit dosage amount varies within this range depending upon the dosage form employed and the route of administrationutilized.

In any of the aforementioned aspects are further embodiments in which the effective amount of the compound described herein, or a pharmaceutically acceptable salt thereof, is: (a) systemically administered to the mammal; and/or (b) administered orally to the mammal; and/or (c) intravenously administered to the mammal; and/or (d) administered by injection to the mammal; and/or (e) administered topically to the mammal; and/or (f) administered non-systemically or locally to the mammal.

In any of the aforementioned aspects are further embodiments comprising single administrations of the effective amount of the compound, including further embodiments in which (i) the compound is administered once a day; or (ii) the compound is administered to the mammal multiple times over the span of one day.

In any of the aforementioned aspects are further embodiments comprising multiple administrations of the effective amount of the compound, including further embodiments in which (i) the compound is administered continuously or intermittently: as in a single dose; (ii) the time between multiple administrations is every 6 hours; (iii) the compound is administered to the mammal every 8 hours; (iv) the compound is administered to the mammal every 12 hours; (v) the compound is administered to the mammal every 24 hours. In further or alternative embodiments, the method comprises a drug holiday, wherein the administration of the compound is temporarily suspended or the dose of the compound being administered is temporarily reduced; at the end of the drug holiday, dosing of the compound is resumed. In one embodiment, the length of the drug holiday varies from 2 days to 1 year.

It is understood that the dosage regimen to treat, prevent, or ameliorate the condition(s) for which relief is sought, is modified in accordance with a variety of factors (e.g., the disease, disorder, or condition from which the subject suffers; the age, weight, sex, diet, and medical condition of the subject). Thus, in some instances, the dosage regimen actually employed varies and, in some embodiments, deviates from the dosage regimens set forth herein.

The compounds described herein, or a pharmaceutically acceptable salt thereof, as well as combination therapies, are administered before, during or after the occurrence of a disease or condition, and the timing of administering the composition containing a compound varies. Thus, in one embodiment, the compounds described herein are used as a prophylactic and are administered continuously to subjects with a propensity to develop conditions or diseases in order to prevent the occurrence of the disease or condition. In another embodiment, the compounds and compositions are administered to a subject during or as soon as possible after the onset of the symptoms. In specific embodiments, a compound described herein is administered as soon as is practicable after the onset of a disease or condition is detected or suspected, and for a length of time necessary for the treatment of the disease. In some embodiments, the length required for treatment varies, and the treatment length is adjusted to suit the specific needs of each subject. For example, in specific embodiments, a compound described herein or a formulation containing the compound is administered for at least 2 weeks, about 1 month to about 5 years.

### EXAMPLES

The following examples are provided for illustrative purposes only and not to limit the scope of the claims provided herein.

As used above, and throughout the description of the invention, the following abbreviations, unless otherwise indicated, shall be understood to have the following meanings:
acac acetylacetone
ACN or MeCN acetonitrile
AcOH acetic acid
Ac acetyl
Ac₂O acetic anhydride
BINAP 2,2′-bis(diphenylphosphino)-1,1′-binaphthalene
Bn benzyl
BOC or Boc tert-butyl carbamate
i-Bu iso-butyl
t-Bu tert-butyl
Cy cyclohexyl
CDI 1,1-carbonyldiimidazole
CPME Cyclopentyl methyl ether
DBA or dba dibenzylideneacetone
DCE dichloroethane (ClCH₂CH₂Cl)
DCM dichloromethane (CH₂Cl₂)
DIBAL-H diisobutylaluminum hydride
DIPEA or DIEA diisopropylethylamine
DMA N,N-dimethylacetamide
DMAP 4-(N,N-dimethylamino)pyridine
DME 1,2-dimethoxyethane
DMF N,N-dimethylformamide
DMPU N,N′-dimethylpropyleneurea
DMSO dimethylsulfoxide
Dppf or dppf 1,1′-bis(diphenylphosphino)ferrocene
EDC or EDCI N-(3-dimethylaminopropyl)-N′-ethylcarbodiimide hydrochloride
EEDQ 2-Ethoxy-1-ethoxycarbonyl-1,2-dihydroquinoline
eq equivalent(s)
Et ethyl
Et₂O diethyl ether
EtOH ethanol
EtOAc ethyl acetate
HATU 1-[bis(dimethylamino)methylene]-1H-1,2,3-triazolo[4,5-b]pyridinium 3-oxide hexafluorophosphate
HMPA hexamethylphosphoramide
HOBt 1-hydroxybenzotriazole
HPLC high performance liquid chromatography
IBX 2-iodoxybenzoic acid
KOAc potassium acetate
KHMDS potassium bis(trimethylsilyl)amide
NaHMDS sodium bis(trimethylsilyl)amide
LiHMDS lithium bis(trimethylsilyl)amide
LAH lithium aluminum anhydride
LCMS liquid chromatography mass spectrometry
2-MeTHF 2-methyltetrahydrofuran
Me methyl
MeOH methanol
MS mass spectroscopy
Ms mesyl
MTBE methyl tert-butyl ether
NaO^{t}Bu sodium tert-butoxide
NBS N-bromosuccinimide
NIS N-iodosuccinimide
NMM N-methyl-morpholine
NMP N-methyl-pyrrolidin-2-one
NMR nuclear magnetic resonance
OTf trifluoromethanesulfonate
PCC pyridinium chlorochromate
PE petroleum ether
Ph phenyl
PPTS pyridium p-toluenesulfonate
iPr/i-Pr iso-propyl
RP-HPLC reverse-phase high-pressure liquid chromatography
rt room temperature
SEM 2-(trimethylsilyl)ethoxymethyl
TBS tert-butyldimethylsilyl
TBAF tetra-n-butylammonium fluoride
TBAI tetra-n-butylammonium iodide
TEA triethylamine
TFA trifluoroacetic acid
THF tetrahydrofuran
THP tetrahydropyran
TLC thin layer chromatography
TMEDA N,N,N′,N′-tetramethylethylenediamine
TMS trimethylsilyl
TsOH/p-TsOH p-toluenesulfonic acid

### Intermediate 1

### 3-Bromo-2,6-difluoro-5-(trifluoromethyl)phenol

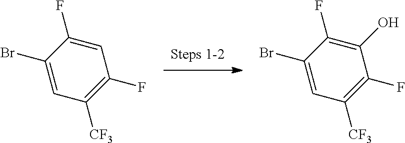 [SMILES: Fc1cc(F)c(C(F)(F)F)cc1Br ; Oc1c(F)c(Br)cc(C(F)(F)F)c1F]

### Step 1: 2-(3-Bromo-2,6-difluoro-5-(trifluoromethyl)phenyl)-4,4,5,5-tetramethyl-1,3,2-dioxaborolane

A mixture of (1,5-cyclooctadiene)(methoxy)iridium(I) dimer (1.13 g, 1.71 mmol), 4,4′-di-tert-butyl-2,2′-bipyridine (0.46 g, 1.71 mmol), and bis(pinacolato)diboron (23.9 g, 94 mmol) was degassed by vacuum/N₂ cycles three times. Cyclopentyl methyl ether (90 mL) was added, and the mixture was degassed by three more vacuum/N₂ cycles. 4-Bromo-1,5-difluoro-2-(trifluoromethyl)benzene (22.3 g, 85 mmol) was added under N₂, and the reaction heated at 100° C. overnight. The reaction mixture was cooled to room temperature and concentrated under reduced pressure. The residue was purified by flash chromatography (0-20% EtOAc/heptane) to give 2-(3-bromo-2,6-difluoro-5-(trifluoromethyl)phenyl)-4,4,5,5-tetramethyl-1,3,2-dioxaborolane (29.3 g, 84%) as a white solid. ¹H NMR (400 MHz, DMSO-d₆): δ 8.32 (t, J=7.4 Hz, 1H), 1.32 (s, 12H).

### Step 2: 3-Bromo-2,6-difluoro-5-(trifluoromethyl)phenol

Hydrogen peroxide (69 mL, 30 w/w in H₂O) was added slowly to a solution of 2-(3-bromo-2,6-difluoro-5-(trifluoromethyl)phenyl)-4,4,5,5-tetramethyl-1,3,2-dioxaborolane (23.6 g, 61 mmol) in methanol (240 mL). The clear solution was stirred at room temperature for 5 h, quenched by the slow dropwise addition of saturated aqueous Na₂S₂O₃ solution over ˜1 h, stirred for additional 30 min, and then extracted twice with EtOAc. The combined organic layers were washed with brine, dried (MgSO₄), and then concentrated under reduced pressure. The residue was purified by silica gel chromatography (0-20% EtOAc in heptane) to yield 3-bromo-2,6-difluoro-5-(trifluoromethyl)phenol as a semi-solid (16.9 g, 73%). ¹H NMR (400 MHz, DMSO-d₆): δ 11.62 (s, 1H), 7.56 (t, J=6.8 Hz, 1H).

### Intermediate 1.01

### 2,6-Difluoro-3-iodo-5-(trifluoromethyl)phenol

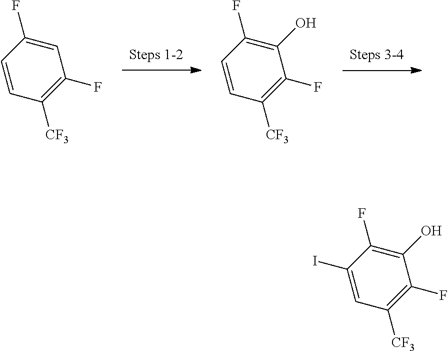 [SMILES: Fc1ccc(C(F)(F)F)c(F)c1 ; Oc1c(F)ccc(C(F)(F)F)c1F ; Oc1c(F)c(I)cc(C(F)(F)F)c1F]

### Step 1: (2,6-Difluoro-3-(trifluoromethyl)phenyl)boronic Acid

n-Butyllithium (2.5 Min hexanes, 171 mL, 428 mmol) was added dropwise to a mixture of 2,4-difluoro-1-(trifluoromethyl)benzene (60.0 g, 329 mmol) in Et₂O (˜400 mL) at −78° C. under N₂. The reaction was stirred for 1 h. Trimethyl borate (44.7 mL, 395 mmol) in Et₂O (200 mL) was added dropwise at −78° C. The reaction was stirred for 1 h, allowed to warm to rt, stirred for 10 h, and then quenched slowly with aq. HCl (1 M, 500 mL) under ice cooling. The organic layer was separated and washed with brine (300 mL) to give (2,6-difluoro-3-(trifluoromethyl)phenyl)boronic acid as a solution in Et₂O (˜600 mL). LCMS: 225.1 [M−H]⁻.

### Step 2: 2,6-Difluoro-3-(trifluoromethyl)phenol

Hydrogen peroxide (166 mL, 1.72 mol, 30% purity in H₂O) was added to a solution of (2,6-difluoro-3-(trifluoromethyl)phenyl)boronic acid (74.4 g, 329 mmol) in Et₂O (˜600 mL) at 0° C. The mixture was heated to 40° C., stirred for 4 h, and then allowed to cool to rt. The aqueous layer was separated. The organic layer was cooled to 0° C. and then quenched with aqueous Na₂SO₃ (20% in H₂O, ˜500 mL) keeping the temperature <20° C. The organic layer was separated. The aqueous layer was extracted with EtOAc (2×300 ml). The organic layers were combined, washed with water (2×300 ml), washed with brine (300 ml), dried (Na₂SO₄), filtered, concentrated and then purified by silica gel chromatography (petroleum ether/ethyl acetate=50:1 to 5:1) to give 2,6-difluoro-3-(trifluoromethyl)phenol (41.3 g, 63%) as a yellow oil. ¹H NMR (400 MHz, DMSO-d₆): δ 11.01 (s, 1H), 7.27-7.19 (m, 2H); LCMS: 196.9 [M−H]⁻.

### Step 3: 2-(Benzyloxy)-1,3-difluoro-4-(trifluoromethyl)benzene

Benzyl bromide (43.2 mL, 363 mmol) was added to a mixture of 2,6-difluoro-3-(trifluoromethyl)phenol (60.3 g, 303 mmol), K₂CO₃ (126 g, 909 mmol), and DMF (600 mL) at rt. The mixture was stirred at 50° C. for 12 h, cooled to rt, poured into H₂O (500 mL) slowly, and then extracted with EtOAc (3×300 mL). The organic layers were combined, washed with brine (300 mL), dried (Na₂SO₄), filtered, concentrated, and then purified by silica gel chromatography (petroleum ether/ethyl acetate=100:1 to 10:1) to give 2-(benzyloxy)-1,3-difluoro-4-(trifluoromethyl)benzene (54.5 g, 62%) as a yellow oil. ¹H NMR (400 MHz, DMSO-d₆): δ 7.56-7.50 (m, 1H), 7.43-7.34 (m, 6H), 5.24 (s, 2H).

### Step 4: 3-(Benzyloxy)-2,4-difluoro-1-iodo-5-(trifluoromethyl)benzene

n-Butyllithium (2.5 M in hexanes, 104 mL, 260 mmol) was added dropwise to a mixture of 2-(benzyloxy)-1,3-difluoro-4-(trifluoromethyl)benzene (50.1 g, 173 mmol) in THF (300 mL) at −78° C. under N₂. The mixture was stirred for 1 h. Iodine (88.1 g, 347 mmol) in THF (200 mL) was added dropwise into the mixture at −78° C. The mixture was allowed to warm to rt, stirred for 12 h, diluted with sat. aq. Na₂SO₃ (500 mL), and then extracted with EtOAc (3×300 mL). The organic layer was washed with brine (300 mL), dried (Na₂SO₄), filtered, concentrated, and then purified by silica gel chromatography (petroleum ether/EtOAc=1/0) to give 3-(benzyloxy)-2,4-difluoro-1-iodo-5-(trifluoromethyl)benzene (64.3 g, 89%) as a yellow oil. ¹H NMR (400 MHz, DMSO-d₆): δ 7.93 (t, 1H), 7.44-7.35 (m, 5H), 5.25 (s, 2H).

### Step 5: 2,6-Difluoro-3-iodo-5-(trifluoromethyl)phenol

Boron tribromide (58.2 mL, 604 mmol) was added dropwise to a mixture of 3-(benzyloxy)-2,4-difluoro-1-iodo-5-(trifluoromethyl)benzene (50.2 g, 121 mmol) in DCM (500 mL) at −78° C. under N₂. The mixture was stirred at rt for 4 h, quenched slowly with MeOH (˜200 mL) at 0° C., concentrated, and then purified by silica gel chromatography (petroleum ether/EtOAc=50:1 to 5:1) to give 2,6-difluoro-3-iodo-5-(trifluoromethyl)phenol (34.6 g, 86%) as a red solid. ¹H NMR (400 MHz, DMSO-d₆): δ 11.39 (s, 1H), 7.59 (t, 1H); LCMS: 322.9 [M−H]⁻.

### Intermediate 1.02

### 3-(Benzyloxy)-2,4-difluoro-5-(trifluoromethyl)aniline

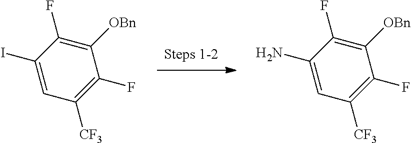 [SMILES: Fc1c(I)cc(C(F)(F)F)c(F)c1OCc1ccccc1 ; Nc1cc(C(F)(F)F)c(F)c(OCc2ccccc2)c1F]

### Step 1: 3-(Benzyloxy)-N-(diphenylmethylene)-2,4-difluoro-5-(trifluoromethyl)aniline

Pd₂(dba)₃ (2.21 g, 2.41 mmol) was added to a mixture of Intermediate 1.01, Step 4 (10.0 g, 24.2 mmol), diphenylmethanimine (8.75 g, 48.3 mmol), BINAP (3.01 g, 4.83 mmol), Cs₂CO₃ (23.6 g, 72.4 mmol), and dioxane (200 mL) under N₂. The mixture was degassed and purged with N₂ 3 times, stirred at 90° C. for 12 h, allowed to cool to rt, poured into H₂O (200 mL), and then extracted with EtOAc (3×100 mL). The organic layer was washed with brine (100 mL), dried (Na₂SO₄), filtered, concentrated, and then purified by silica gel chromatography (petroleum ether/ethyl acetate=50:1 to 5:1) to give 3-(benzyloxy)-N-(diphenylmethylene)-2,4-difluoro-5-(trifluoromethyl)aniline (11.6 g, 71%) as a yellow oil. ¹H NMR (400 MHz, DMSO-d₆): δ 7.69 (d, 2H), 7.61-7.56 (m, 1H), 7.52-7.48 (m, 2H), 7.40-7.35 (m, 6H), 7.30-7.27 (m, 2H), 7.17-7.15 (m, 2H), 6.96 (t, 1H), 5.09 (s, 2H); LCMS: 468.1 [M+H]⁺.

### Step 2: 3-(Benzyloxy)-2,4-difluoro-5-(trifluoromethyl)aniline

A mixture of 3-(benzyloxy)-N-(diphenylmethylene)-2,4-difluoro-5-(trifluoromethyl)aniline (11.6 g, 24.8 mmol) and 4 M HCl in EtOAc (200 mL) was stirred at rt for 2 h, adjusted to pH=˜7 with sat. aq. NaHCO₃, and then extracted with EtOAc (3×100 mL). The organic layer was washed with brine (100 mL), dried (Na₂SO₄), filtered, concentrated, and then purified by silica gel chromatography (petroleum ether/ethyl acetate=50:1 to 5:1) to give 3-(benzyloxy)-2,4-difluoro-5-(trifluoromethyl)aniline (2.6 g, 34%) as a yellow oil. ¹H NMR (400 MHz, DMSO-d₆): δ 7.43-7.33 (m, 5H), 6.79 (t, 1H), 5.53 (s, 2H), 5.17 (s, 2H); LCMS: 304.0 [M+H]⁺.

### Intermediate 1.03

### 4-(Benzyloxy)-3,5-difluoro-2-iodo-6-(trifluoromethyl)pyridine

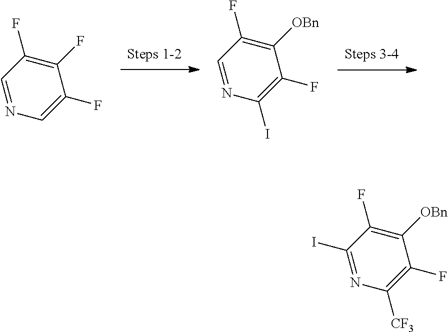 [SMILES: Fc1cncc(F)c1F ; Fc1cnc(I)c(F)c1OCc1ccccc1 ; Fc1c(I)nc(C(F)(F)F)c(F)c1OCc1ccccc1]

### Step 1: 4-(Benzyloxy)-3,5-difluoropyridine

Sodium hydride (1.32 g, 33.1 mmol, 60%) was added slowly to a mixture of 3,4,5-trifluoropyridine (4.01 g, 30.1 mmol), BnOH (3.58 g, 33.1 mmol), and DMF (50 mL) at rt under N₂. The mixture was stirred for 1 h, poured into H₂O (40 mL) slowly, and then extracted with ethyl acetate (4×20 mL). The organic layer was washed with brine (20 mL), dried over Na₂SO₄, filtered, concentrated, and then purified by silica gel chromatography (petroleum ether:ethyl acetate=20:1 to 13:1) to give 4-(benzyloxy)-3,5-difluoropyridine (6.20 g 93%) as a colorless liquid. ¹H NMR (400 MHz, CDCl₃): δ 8.25 (s, 2H), 7.47-7.34 (m, 5H), 5.42 (s, 2H); LCMS: 222.1 [M+H]⁺.

### Step 2: 4-(Benzyloxy)-3,5-difluoro-2-iodopyridine

n-Butyllithium (2.5 M in n-hexane, 7.05 mL, 17.6 mmol) was added dropwise to a mixture of 4-(benzyloxy)-3,5-difluoropyridine (3.02 g, 13.6 mmol) in THF (35 mL) at −78° C. under N₂. The mixture was stirred for 1 h. Iodine (5.16 g, 20.3 mmol) in THF (10 mL) was added dropwise at −78° C. under N₂. The mixture was stirred for 1 h, allowed to warm to rt, added into sat. aq. Na₂SO₃ (80 mL) slowly, and then extracted with ethyl acetate (4×20 mL). The organic layer was washed with brine (80 mL), dried over Na₂SO₄, filtered, concentrated, and then purified by column chromatography (SiO₂, petroleum ether/ethyl acetate=50/1 to 20/1) to give 4-(benzyloxy)-3,5-difluoro-2-iodopyridine (1.80 g, 38%) as a yellow solid. ¹H NMR (400 MHz, CDCl₃): δ 8.10 (s, 1H), 7.46-7.35 (m, 5H), 5.41 (s, 2H); LCMS: 347.9 [M+H]⁺.

### Step 3: 4-(Benzyloxy)-3,5-difluoro-2-(trifluoromethyl)pyridine

Methyl 2,2-difluoro-2-(fluorosulfonyl)acetate (5.45 g, 28.4 mmol) and CuI (5.40 g, 28.4 mmol) were added to a solution of 4-(benzyloxy)-3,5-difluoro-2-iodopyridine (1.97 g, 5.68 mmol) in DMF (20 mL) under N₂. The mixture was stirred at 70° C. for 4 h, allowed to cool to rt, and then filtered. The filtrate was diluted with aqueous NH₃·H₂O (100 mL, 9% aq. solution) and then diluted with ethyl acetate (20 mL). The layers were separated. The aqueous layer was extracted with additional ethyl acetate (10 mL). The combined organic layers were washed with aqueous NH₃·H₂O (3×20 mL, 9% aq. solution), washed with water (50 mL), washed with brine (50 mL), dried over anhydrous Na₂SO₄, filtered, concentrated, and then purified by silica gel chromatography (petroleum ether/ethyl acetate=1/0 to 10/1) to give 4-(benzyloxy)-3,5-difluoro-2-(trifluoromethyl)pyridine (1.30 g, 79%) as a colorless liquid. ¹H NMR (400 MHz, CDCl₃): δ 8.30 (s, 1H), 7.49-7.34 (m, 5H), 5.48 (s, 2H); LCMS: 290.0 [M+H]⁺.

### Step 4: 4-(Benzyloxy)-3,5-difluoro-2-iodo-6-(trifluoromethyl)pyridine

Lithium diisopropylamide (2 M in THF, 1.40 mL, 2.8 mmol) was added dropwise to a mixture of 4-(benzyloxy)-3,5-difluoro-2-(trifluoromethyl)pyridine (0.54 g, 1.87 mmol) and THF (10 mL) at −78° C. under N₂. The reaction was stirred for 1 h. Iodine (711 mg, 2.80 mmol) in THF (5 mL) was added dropwise. The mixture was stirred at −78° C. for 1 h, poured into sat. aq. Na₂SO₃ (˜20 mL) slowly, and then extracted with ethyl acetate (3×15 mL). The organic layer was washed with brine (20 mL), dried over Na₂SO₄, filtered, concentrated, and then purified by silica gel chromatography (petroleum ether) to give 4-(benzyloxy)-3,5-difluoro-2-iodo-6-(trifluoromethyl)pyridine (300 mg, 38%) as a yellow solid. ¹H NMR (400 MHz, CDCl₃): δ 7.50-7.35 (m, 5H), 5.47 (s, 2H); LCMS: 416.0 [M+H]⁺.

### Intermediate 1.04

### 3-Bromo-6-chloro-2-fluoro-5-(trifluoromethyl)phenol

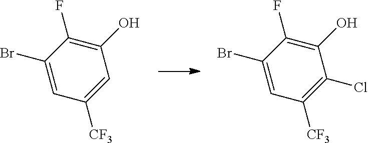 [SMILES: Oc1cc(C(F)(F)F)cc(Br)c1F ; Oc1c(F)c(Br)cc(C(F)(F)F)c1Cl]

1,3-Dichloro-5,5-dimethylhydantoin (5.52 g, 19.3 mmol) was added to the mixture of 3-bromo-2-fluoro-5-(trifluoromethyl)phenol (5.00 g, 19.3 mmol) and diisopropylamine HCl (27 mg, 0.19 mmol) in toluene at 0° C. The yellow suspension was stirred at 0° C. in the absence of light for 2 h, diluted with water, and then extracted with ethyl acetate. The organic layer was dried (MgSO₄), concentrated, and then purified by silica gel chromatography (0-50% DCM in heptane). The crude material was purified further by prep-HPLC (40-100% CH₃CN in water with 0.1% TFA). The fractions were combined, concentrated, diluted with ethyl acetate, and then washed with sat. aq. NaHCO₃. The aqueous layer was back extracted with ethyl acetate. The combined organics were washed with brine, dried (MgSO₄), filtered, and then concentrated to give 3-bromo-6-chloro-2-fluoro-5-(trifluoromethyl)phenol (3.3 g, 55%) as a white semi-solid. ¹H NMR (400 MHz, DMSO-d₆): δ 6.49-6.42 (m, 1H).

### Intermediate 2

### 2-(3,4-Difluoro-5-(methoxymethoxy)phenyl)-4,4,5,5-tetramethyl-1,3,2-dioxaborolane

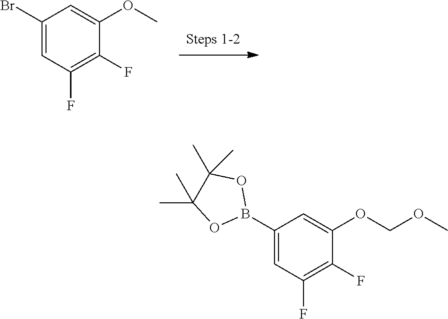

### Step 1: 5-Bromo-1,2-difluoro-3-(methoxymethoxy)benzene

Boron tribromide (108 mL, 1.12 mol) was added dropwise to a solution of 5-bromo-1,2-difluoro-3-methoxybenzene (50 g, 224 mmol) in DCM (500 mL) at −78° C. The reaction mixture was stirred at room temperature for 2 h, slowly added into MeOH (500 mL), stirred for 0.5 h, poured into saturated NaHCO₃ (2000 mL) and then extracted (3×2000 mL EtOAc). The combined organic layers were washed (2000 mL brine), dried (Na₂SO₄), filtered, and then concentrated. The residue was purified by silica gel chromatography (petroleum ether) to give the intermediate product 5-bromo-2,3-difluorophenol (31 g, 66%) as a yellow oil. The oil was dissolved in DCM (500 mL) and cooled in an ice bath. DIEA (38.8 mL, 223 mmol) and then chloro(methoxy)methane (13.6 mL, 180 mmol) were added dropwise. The reaction mixture was stirred at room temperature for an additional 2 h, poured into H₂O (500 mL), and then extracted (3×500 mL DCM). The combined organic layers were washed (500 mL brine), dried (Na₂SO₄), filtered, and then concentrated. The residue was purified by silica gel chromatography (petroleum ether) to give 5-bromo-1,2-difluoro-3-(methoxymethoxy)benzene (30 g, 79%) as a yellow oil. ¹H NMR (400 MHz, DMSO-d₆): δ 7.38 (ddd, 1H), 7.31 (td, 1H), 5.31 (s, 2H), 3.38 (s, 3H).

### Step 2: 2-(3,4-Difluoro-5-(methoxymethoxy)phenyl)-4,4,5,5-tetramethyl-1,3,2-dioxaborolane

Pd(dppf)Cl₂ (7.81 g, 10.7 mmol) was added to a mixture of 5-bromo-1,2-difluoro-3-(methoxymethoxy)benzene (27 g, 107 mmol), bis(pinacolato)diboron (40.6 g, 160 mmol), and KOAc (62.8 g, 640 mmol) in toluene (300 mL) at room temperature under N₂. The mixture was degassed with 3 vacuum/N₂ cycles, stirred at 90° C. overnight, allowed to cool to room temperature, poured into H₂O (500 mL), and then extracted (3×500 mL EtOAc). The combined organic layers were washed (1000 mL brine), dried (Na₂SO₄), filtered, and then concentrated. The residue was purified by silica gel chromatography (2% EtOAc/petroleum ether) to give 2-(3,4-difluoro-5-(methoxymethoxy)phenyl)-4,4,5,5-tetramethyl-1,3,2-dioxaborolane (27 g, 84%) as a white solid. ¹H NMR (400 MHz, DMSO-d₆): 7.37 (d, 1H), 7.26 (dd, 1H), 5.36 (s, 2H), 3.48 (s, 3H), 1.35 (s, 12H).

The Intermediate below was synthesized in a similar manner to that described for Intermediate 2.

| Int | Structure | Name | ¹H NMR (400 MHz, DMSO-d₆) |
|---|---|---|---|
| 2.01 | 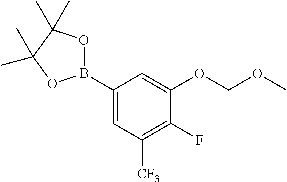 | 2-(4-Fluoro-3- (methoxymethoxy)-5- (trifluoromethyl)phenyl)- 4,4,5,5-tetramethyl-1,3,2- dioxaborolane | δ 7.75 (d, 1H), 7.51 (d, 1H), 5.36 (s, 2H), 3.42 (s, 3H), 1.16 (s, 12H) |

### Intermediate 3

### 2-Bromo-3-fluoro-4-(methoxymethoxy)pyridine

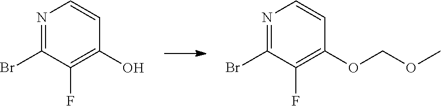

Methoxymethyl chloride (254 mg, 3.15 mmol) was added dropwise to a solution of 2-bromo-3-fluoropyridin-4-ol (500 mg, 2.60 mmol) and DIPEA (505 mg, 3.91 mmol) in DCM (10 mL) at 0° C. The mixture was warmed to room temperature, stirred overnight, slowly poured into H₂O (30 mL), and then extracted (3×30 mL DCM). The combined organic layers were washed (70 mL brine), dried (Na₂SO₄), filtered, and then concentrated. The residue was purified by silica gel chromatography (10% EtOAc/petroleum ether) to give 2-bromo-3-fluoro-4-(methoxymethoxy)pyridine (245 mg, 39%) as a colorless oil. ¹H NMR (400 MHz, DMSO-d₆): δ 8.10 (d, 1H), 7.35 (t, 1H), 5.42 (s, 2H), 3.42 (s, 3H).

The Intermediates below were synthesized in a similar manner to that described for Intermediate 3.

| Int | Structure | Name | ¹H NMR (400 MHz) |
|---|---|---|---|
| 3.01 | 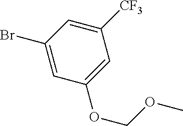 | 1-Bromo-3- (methoxymethoxy)-5- (trifluoromethyl)benzene | (DMSO-d₆): δ 7.57-7.56 (m, 2H), 7.36-7.35 (m, 1H), 5.32 (s, 2H), 3.39 (s, 3H) |
| 3.02 | 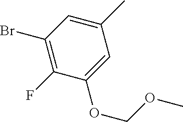 | 1-Bromo-2-fluoro-3- (methoxymethoxy)-5- methylbenzene | (CDCl₃): δ 7.01-6.99 (m, 1H), 6.95-6.93 (m, 1H), 5.19 (s, 2H), 3.52 (s, 3H), 2.29 (s, 3H) |
| 3.03 | 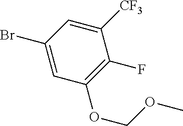 [SMILES: COCOc1cc(Br)cc(C(F)(F)F)c1F] | 5-Bromo-2-fluoro-1- (methoxymethoxy)-3- (trifluoromethyl)benzene | (DMSO-d₆): δ 7.80 (dd, 1H), 7.58 (dd, 1H), 5.38 (s, 2H), 3.42 (s, 3H) |
| 3.04 | 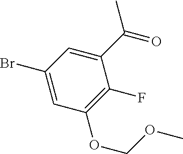 | 1-(5-Bromo-2-fluoro-3- (methoxymethoxy)phenyl) ethanone | (DMSO-d₆): δ 7.67 (dd, J = 2.4, 7.0 Hz, 1H), 7.52 (dd, J = 2.4, 5.4 Hz, 1H), 5.35 (s, 2H), 3.43 (s, 3H), 2.58 (d, J = 3.9 Hz, 3H) |

### Intermediate 4

### 2-Bromo-5-(methoxymethoxy)pyridine

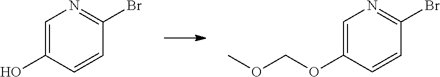

LiHMDS (1 M, 5.8 mL) was added dropwise via syringe to a solution of 6-bromopyridin-3-ol (1.00 g, 5.75 mmol) in THF (10 mL) at 0° C. The reaction mixture was stirred at 0° C. for 20 min under N₂. Methoxymethyl chloride (555 mg, 6.90 mmol) was added dropwise to the reaction mixture at 0° C. The reaction mixture was warmed to room temperature, stirred for 15 h, carefully poured into H₂O (30 mL), and then extracted (3×40 mL EtOAc). The combined organic layers were washed (20 mL brine), dried (Na₂SO₄), filtered, and then concentrated. The residue was purified by silica gel chromatography (5-20% EtOAc/petroleum ether) to give 2-bromo-5-(methoxymethoxy)pyridine (550 mg, 43%) as a yellow oil. ¹H NMR (400 MHz, DMSO-d₆): δ 8.16 (d, 1H), 7.56 (d, 1H), 7.45 (d, 1H), 5.26 (s, 2H), 3.38 (s, 3H); LCMS: 218.0 [M+H]⁺.

The Intermediates below were synthesized in a similar manner to that described for Intermediate 4.

| Int | Structure | Name | [M + H]⁺ |
|---|---|---|---|
| 4.01 | 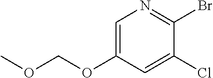 | 2-Bromo-3-chloro-5- (methoxymethoxy)pyridine | 252.0 |
| 4.02 | 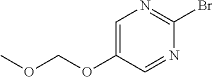 | 2-Bromo-5- (methoxymethoxy)pyrimidine | 219.0 |
| 4.03 | 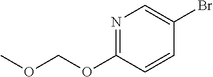 | 5-Bromo-2- (methoxymethoxy)pyridine | 218.1 |

### Intermediate 4.04

### 5-Bromo-1-(1,1-difluoroethyl)-2-fluoro-3-(methoxymethoxy)benzene

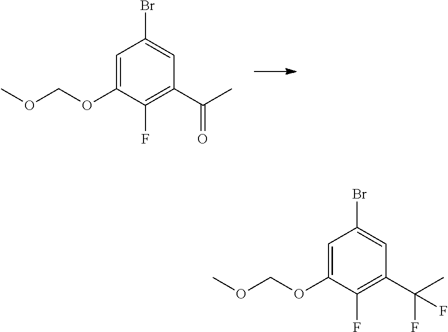 [SMILES: COCOc1cc(Br)cc(C(C)=O)c1F ; COCOc1cc(Br)cc(C(C)(F)F)c1F]

(Diethylamino)sulfur trifluoride (0.44 mL, 3.32 mmol) was added slowly to a solution of 1-acetyl-5-bromo-2-fluoro-3-(methoxymethoxy)benzene (0.12 g, 0.42 mmol) in DCM (1.00 mL) at 0° C. The reaction mixture was allowed to warm up to rt, stirred at rt overnight, stirred at 50° C. for 5 h, stirred at 40° C. over the weekend, added to ice, and then extracted with DCM. The organics were dried (MgSO₄), concentrated, and then purified by column chromatography (0-20% EtOAc in heptane) to give 5-bromo-1-(1,1-difluoroethyl)-2-fluoro-3-(methoxymethoxy)benzene (85 mg, 65%) as a yellow oil. ¹H NMR (400 MHz, DMSO-d₆): δ 7.62 (dd, J=2.3, 7.1 Hz, 1H), 7.31 (dd, J=2.3, 5.7 Hz, 1H), 5.34 (s, 2H), 3.42 (s, 3H), 2.01 (t, J=19.2 Hz, 3H).

### Intermediate 5

### 3-(4-Bromophenoxy)propanoic acid

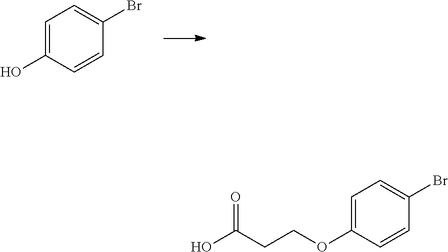 [SMILES: Oc1ccc(Br)cc1 ; O=C(O)CCOc1ccc(Br)cc1]

A mixture of 4-bromophenol (10.0 g, 57.8 mmol), 3-chloropropanoic acid (6.27 g, 57.8 mmol), NaOH (5.55 g, 139 mmol), and water (30 mL) was stirred at reflux overnight and then cooled to room temperature. The pH was adjusted to pH-1 with conc. HCl, and the mixture was extracted (3×20 mL EtOAc). The combined organic layers were dried (Na₂SO₄), filtered, and then concentrated. The residue was stirred in EtOH (7 mL) at 60° C. for 0.5 h, slowly cooled to room temperature, and then stirred overnight. The mixture was filtered, and the filter cake was washed with ice-cold EtOH (2 mL) to give 3-(4-bromophenoxy)propanoic acid (1.5 g, 10%) as a white solid. ¹H NMR (400 MHz, DMSO-d₆): δ 12.38 (s, 1H), 7.44 (d, 2H), 6.91 (d, 2H), 4.15 (t, 2H), 2.68 (t, 2H); LCMS: 242.9 [M−H]⁻.

### Intermediate 6

### 1-(Isopropylsulfonyl)piperazine Hydrochloride

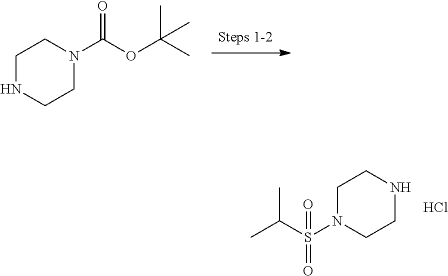

### Step 1: tert-Butyl-4-(isopropylsulfonyl)piperazine-1-carboxylate

Propane-2-sulfonyl chloride (766 mg, 5.37 mmol) was added to a solution of tert-butyl piperazine-1-carboxylate (1.0 g, 5.37 mmol) and Na₂CO₃ (683 mg, 6.44 mmol) in acetonitrile (10 mL) at room temperature under N₂. The mixture was stirred overnight, slowly poured into H₂O (50 mL), and then extracted (3×50 mL EtOAc). The combined organic layers were washed (30 mL brine), dried (Na₂SO₄), filtered, and then concentrated. The residue was purified by silica gel chromatography (20% EtOAc/petroleum ether) to give tert-butyl-4-(isopropylsulfonyl)piperazine-1-carboxylate (1.2 g, 76%) as a yellow oil. ¹H NMR (400 MHz, DMSO-d₆): δ 3.36-3.25 (m, 5H), 3.25-3.15 (m, 4H), 1.39 (s, 9H), 1.20 (s, 6H).

### Step 2: 1-(Isopropylsulfonyl)piperazine Hydrochloride

A mixture of tert-butyl-4-(isopropylsulfonyl)piperazine-1-carboxylate (1.2 g, 4.10 mmol) and HCl in MeOH (4M, 40 mL) was stirred at room temperature for 2 h. The solvent was evaporated under vacuum to give 1-(isopropylsulfonyl)piperazine hydrochloride (850 mg) as a white solid. ¹H NMR (400 MHz, DMSO-d₆): δ 9.60-9.30 (m, 2H), 3.51-3.31 (s, 5H), 3.10 (s, 4H), 1.22 (d, 6H).

The Intermediates below were synthesized in a similar manner to that described for Intermediate 6.

| Int | Structure | Name | ¹H NMR (400 MHz, DMSO-d₆): |
|---|---|---|---|
| 6.01 | 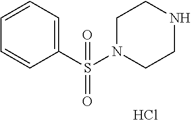 [SMILES: O=S(=O)(c1ccccc1)N1CCNCC1 ; Cl] | 1-(Phenylsulfonyl)piperazine hydrochloride | δ 9.45 (s, 2H), 7.80-7.70 (m, 3H), 7.69-7.60 (m, 2H), 3.15 (s, 8H) |
| 6.02 | 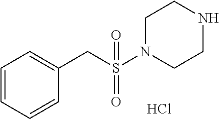 [SMILES: O=S(=O)(Cc1ccccc1)N1CCNCC1 ; Cl] | 1-(Benzylsulfonyl)piperazine hydrochloride | δ 9.65 (s, 2H), 7.50-7.25 (m, 5H), 4.55 (s, 2H), 3.45-3.25 (m, 4H), 3.10-2.90 (m, 4H) |
| 6.03 | 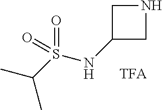 | N-(Azetidin-3-yl)-propane-2- sulfonamide 2,2,2- trifluoroacetate | δ 8.90-8.55 (m, 2H), 8.05 (d, 1H), 4.21-4.15 (m, 1H), 4.12-4.02 (m, 2H), 3.90-3.78 (m, 2H), 3.20-3.10 (m, 1H), 1.21 (d, 6H) |
| 6.04 | 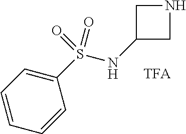 [SMILES: O=S(=O)(NC1CNC1)c1ccccc1] | N-(Azetidin-3- yl)benzenesulfonamide 2,2,2- trifluoroacetate | δ 8.90-8.61 (m, 2H), 8.61 (d, 1H), 7.90-7.75 (m, 2H), 7.75-7.62 (m, 1H), 7.62-7.55 (m, 2H), 4.30-4.10 (m, 1H), 3.99-3.82 (m, 2H), 3.78- 3.60 (m, 2H) |
| 6.05 | 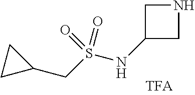 [SMILES: O=S(=O)(CC1CC1)NC1CNC1] | N-(Azetidin-3-yl)-1- cyclopropylmethanesulfonamide 2,2,2-trifluoroacetate | δ 8.90-8.65 (m, 2H), 8.08 (d, 1H), 4.45-4.30 (m, 1H), 4.21-4.08 (m, 2H), 3.98-3.81 (m, 2H), 3.00 (d, 2H), 0.99-0.89 (m, 1H), 0.61-0.49 (m, 2H), 0.35-0.21 (m, 2H) |
| 6.06 | 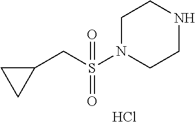 [SMILES: O=S(=O)(CC1CC1)N1CCNCC1 ; Cl] | 1- ((Cyclopropylmethyl)sulfonyl) piperazine hydrochloride | δ 9.38 (s, 2H), 3.51-3.38 (m, 4H), 3.22-3.05 (m, 6H), 1 02-0.88 (m, 1H), 0.62-0.55 (m, 2H), 0.42-0.25 (m, 2H) |
| 6.07 (1) | 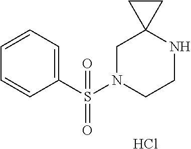 [SMILES: O=S(=O)(c1ccccc1)N1CCNC2(CC2)C1 ; Cl] | 7-(Phenylsulfonyl)-4,7- diazaspiro[2.5]octane hydrochloride | δ 9.56 (br s, 2H), 7.82-7.74 (m, 3H), 7.73-7.67 (m, 2H), 3.23 (br d, J = 6.4 Hz, 4H), 3.07 (s, 2H), 1.11-1.05 (m, 2H), 0.91-0.84 (m, 2H) |
| 6.08 (1) | 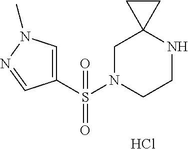 [SMILES: Cn1cc(S(=O)(=O)N2CCNC3(CC3)C2)cn1 ; Cl] | 7-((1-Methyl-1H-pyrazol-4- yl)sulfonyl)-4,7- diazaspiro[2.5]octane hydrochloride | δ 9.66 (br s, 2H), 8.40 (s, 1H), 7.84 (d, J = 0.6 Hz, 1H), 3.93 (s, 3H), 3.34-3.24 (m, 2H), 3.20-3.12 (m, 2H), 3.01 (s, 2H), 1.15-1.06 (m, 2H), 0.94-0.85 (m, 2H) |

Alternate conditions used: 1. Step 1: TEA, DCM, 0° C.-rt, 1 h; Step 2: 4 M HCl in dioxane, dioxane, rt, overnight.

### Intermediate 7

### 5-Chloro-1H-pyrazolo[3,4-c]pyridazine

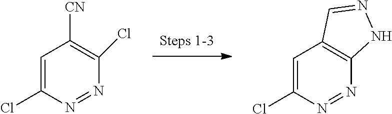 [SMILES: N#Cc1cc(Cl)nnc1Cl ; Clc1cc2cn[nH]c2nn1]

### Step 1: 5-Chloro-1H-pyrazolo[3,4-c]pyridazin-3-amine

A mixture of 3,6-dichloropyridazine-4-carbonitrile (2.0 g, 11.5 mmol), hydrazine hydrate (2.71 g, 46.0 mmol, 85% in water) and MeOH (20 mL) was stirred at 60° C. for 2 h, allowed to cool to room temperature and then filtered. The filter cake was dried under vacuum to give 5-chloro-1H-pyrazolo[3,4-c]pyridazin-3-amine (1.6 g) as yellow solid. ¹H NMR (400 MHz, DMSO-d₆): δ 8.25 (s, 1H), 6.12 (s, 2H); LCMS: 170.1 [M+H]⁺.

### Step 2: 5-Chloro-1H-pyrazolo[3,4-c]pyridazine-3-diazonium Acetate

A solution of NaNO₂ (814 mg, 11.8 mmol) in H₂O (5 mL) was added dropwise to a stirred suspension of 5-chloro-1H-pyrazolo[3,4-c]pyridazin-3-amine (1.0 g, 5.90 mmol) in AcOH (10 mL) at 0° C. The mixture was warmed to room temperature, stirred overnight, and then cooled to 0° C. The solids were collected by filtration and then washed with cold water to give 5-chloro-1H-pyrazolo[3,4-c]pyridazine-3-diazonium acetate (1.07 g) as yellow solid. LCMS: 181.0 M⁺.

### Step 3: 5-Chloro-1H-pyrazolo[3,4-c]pyridazine

A mixture of 5-chloro-1H-pyrazolo[3,4-c]pyridazine-3-diazonium (1.07 g, 5.89 mmol), HCl in H₂O (0.1 M, 60 mL), and DME (10 mL) was heated at 80° C. for 2 h, allowed to cool to room temperature, and then extracted (2×50 mL EtOAc). The combined organic layers were washed (2×30 mL water and then 30 mL brine), dried (Na₂SO₄), filtered, and then concentrated. The residue was purified by silica gel chromatography (20% EtOAc/petroleum ether) to give 5-chloro-1H-pyrazolo[3,4-c]pyridazine (350 mg, 38%) as a yellow solid. ¹H NMR (400 MHz, DMSO-d₆): δ 14.70 (s, 1H), 8.53-8.29 (m, 2H); LCMS: 155.1 [M+H]⁺.

### Intermediate 7.01

### 5-Chloro-4-fluoro-1H-pyrazolo[3,4-c]pyridine

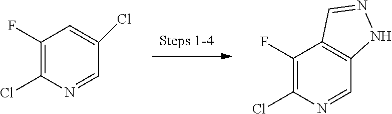 [SMILES: Fc1cc(Cl)cnc1Cl ; Fc1c(Cl)ncc2[nH]ncc12]

### Step 1: tert-Butyl (6-chloro-5-fluoro pyridin-3-yl)carbamate

Xantphos (2.47 g, 4.28 mmol) and Pd₂(dba)₃ (1.96 g, 2.14 mmol) were added to a mixture of 5-bromo-2-chloro-3-fluoropyridine (15 g, 71.3 mmol), tert-butyl carbamate (9.19 g, 78.4 mmol), Cs₂CO₃ (46.5 g, 143 mmol), and dioxane (300 mL) under N₂. The reaction mixture was degassed under vacuum and purged with N₂ 3 times, stirred at 85° C. overnight, allowed to cool to rt, and then filtered through Celite. The Celite pad was washed with EtOAc (800 mL). The filtrate was concentrated and then purified by silica gel chromatography (petroleum ether/EtOAc=9/1) to give tert-butyl (6-chloro-5-fluoropyridin-3-yl)carbamate (14 g, 79%) as a yellow solid. ¹H NMR (400 MHz, DMSO-d₆): δ 10.00 (s, 1H), 8.29 (s, 1H), 7.98 (dd, 1H), 1.48 (s, 9H); LCMS: 247.1 [M+H]⁺.

### Step 2: tert-Butyl (6-chloro-5-fluoro-4-methylpyridin-3-yl)carbamate

n-Butyllithium (2.5 M in n-hexane, 70 mL, 175 mmol) was added dropwise to a solution of tert-butyl (6-chloro-5-fluoropyridin-3-yl)carbamate (16 g, 65 mmol) in THF (160 mL) at −78° C. under N₂. The reaction mixture was stirred at −78° C. for 2 h. Iodomethane (14.7 g, 104 mmol) was added dropwise at −78° C. The reaction mixture was stirred for 3 h, allowed to warm to rt slowly, poured into water (400 mL), and then extracted with MTBE (3×200 mL). The combined organic layers were dried over Na₂SO₄, filtered, concentrated, and then purified by silica gel chromatography (petroleum ether/EtOAc=4/1) to give tert-butyl (6-chloro-5-fluoro-4-methylpyridin-3-yl)carbamate (13.5 g, 80%) as a white solid. ¹H NMR (400 MHz, DMSO-d₆): δ9.14 (s, 1H), 8.28 (s, 1H), 2.19 (d, 3H), 1.47 (s, 9H); LCMS: 261.0 [M+H]⁺.

### Step 3: 6-Chloro-5-fluoro-4-methylpyridin-3-amine hydrochloride

A mixture of tert-butyl (6-chloro-5-fluoro-4-methylpyridin-3-yl)carbamate (13.5 g, 51.8 mmol) and HCl in EtOAc (4 N, 150 mL) was stirred at rt for 2 h. The reaction mixture was filtered, and the filter cake was washed with ice cold EtOAc (50 mL). The cake was dried under high vacuum to give 6-chloro-5-fluoro-4-methylpyridin-3-amine hydrochloride (8 g) as a white solid. ¹H NMR (400 MHz, MeOD-d₄): δ 8.09 (s, 1H), 2.36 (d, 3H); LCMS: 161.0 [M+H]⁺.

### Step 4: 5-Chloro-4-fluoro-1H-pyrazolo[3,4-c]pyridine

Sodium nitrite (2.80 g, 40.6 mmol) was added to a solution of 6-chloro-5-fluoro-4-methylpyridin-3-amine hydrochloride (8 g, 40.6 mmol) in AcOH (100 mL). The reaction mixture was stirred at rt overnight, concentrated, diluted with sat. aq. NaHCO₃ (150 mL), and then extracted with EtOAc (3×50 mL). The combined organic layers were washed with brine (50 mL), dried over Na₂SO₄, filtered, concentrated, and then purified by silica gel chromatography (petroleum ether/EtOAc=80/20 to petroleum ether/EtOAc=1/1) to give 5-chloro-4-fluoro-1H-pyrazolo[3,4-c]pyridine (4.5 g, 50% over 2 steps) as a red solid. ¹H NMR (400 MHz, DMSO-d₆): δ 14.18 (s, 1H), 8.78 (s, 1H), 8.44 (s, 1H); LCMS: 171.9 [M+H]⁺.

### Intermediate 8

### 5-Chloro-3-fluoro-1H-pyrazolo[3,4-c]pyridine

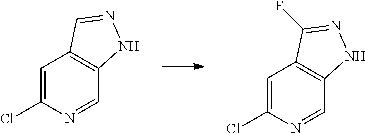 [SMILES: Clc1cc2cn[nH]c2cn1 ; Fc1n[nH]c2cnc(Cl)cc12]

A mixture of 5-chloro-1H-pyrazolo[3,4-c]pyridine (1.70 g, 11.1 mmol), Selectfluor (4.51 g, 12.7 mmol), and acetonitrile (25 mL) was degassed with 3 vacuum/N₂ cycles, heated at 80° C. for 12 h under N₂, allowed to cool to rt, quenched (35 mL water), and then extracted (4×15 mL EtOAc). The combined organic layers were dried (Na₂SO₄), filtered, and then concentrated. The residue was purified by silica gel chromatography (EtOAc/petroleum ether) to give 5-chloro-3-fluoro-1H-pyrazolo[3,4-c]pyridine (1.05 g, 38%) as a light yellow solid. ¹H NMR (400 MHz, DMSO-d₆): δ 13.3 (s, 1H), 8.86 (s, 1H), 7.93 (s, 1H); LCMS: 172.1 [M+H]⁺.

### Intermediate 8.01

### 5-Chloro-3-iodo-1-(tetrahydro-2H-pyran-2-yl)-1H-pyrazolo[4,3-b]pyridine

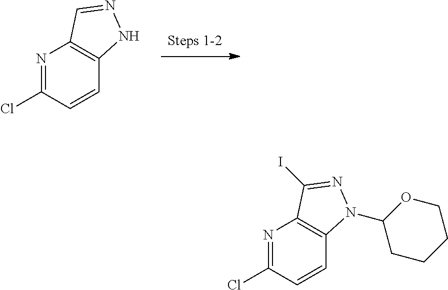 [SMILES: Clc1ccc2[nH]ncc2n1 ; Clc1ccc2c(n1)c(I)nn2C1CCCCO1]

### Step 1: 5-Chloro-3-iodo-1H-pyrazolo[4,3-b]pyridine

Potassium hydroxide (7.31 g, 130 mmol) was added to a solution of 5-chloro-1H-pyrazolo[4,3-b]pyridine (4 g, 26.1 mmol), I₂ (13.2 g, 52.1 mmol), and DMF (80 mL) at 0° C. The mixture was allowed to warm to rt overnight, poured into water (150 mL), and then extracted with EtOAc (2×130 mL). The organic layers were combined, washed with Na₂SO₃ (50 mL), washed with water (2×50 mL), washed with brine (50 mL), dried (Na₂SO₄), filtered, concentrated, and then purified by silica gel chromatography (petroleum ether/EtOAc=20/1) to give 5-chloro-3-iodo-1H-pyrazolo[4,3-b]pyridine (6 g, 82%) as a yellow solid. ¹H NMR (400 MHz, DMSO-d₆): δ 13.76 (br s, 1H), 7.90 (d, 1H), 7.27 (d, 1H); LCMS: 279.9 [M+H]⁺.

### Step 2: 5-Chloro-3-iodo-1-(tetrahydro-2H-pyran-2-yl)-1H-pyrazolo[4,3-b]pyridine

A mixture of 5-chloro-3-iodo-1H-pyrazolo[4,3-b]pyridine (6 g, 21.5 mmol), 3,4-dihydro-2H-pyran (10.8 g, 129 mmol), TsOH—H₂O (817 mg, 4.29 mmol), and DCM (120 mL) was stirred at rt overnight, poured into sat. aq. NaHCO₃ (150 mL), and then extracted with DCM (2×100 mL). The organic layers were combined, washed with water (2×50 mL), washed with brine (15 mL), dried (Na₂SO₄), filtered, concentrated, and then purified by silica gel chromatography (petroleum ether/EtOAc=50/1) to give 5-chloro-3-iodo-1-(tetrahydro-2H-pyran-2-yl)-1H-pyrazolo[4,3-b]pyridine (6 g, 760) as a yellow solid. ¹H-NMR (400 MHz, DMSO-d₆): δ 8.35 (d, 1H), 7.60 (d, 1H), 5.91 (dd, 1H), 3.89-3.82 (m, 1H), 3.80-3.68 (m, 1H), 2.39-2.24 (m, 1H), 2.00 (d, 2H), 1.80-1.65 (m, 1H), 1.64-1.52 (in, 2H), LCMS: 363.9 [M+H]⁺.

The Intermediates below were synthesized in a similar manner to that described for Intermediate 8.01.

| Int | Structure | Name | [M + H]⁺ |
|---|---|---|---|
| 8.02 | 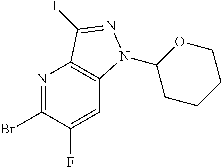 [SMILES: Fc1cc2c(nc1Br)c(I)nn2C1CCCCO1] | 5-Bromo-6-fluoro-3-iodo-1- (tetrahydro-2H-pyran-2-yl)-1H- pyrazolo[4,3-b]pyridine | 425.9 |
| 8.03 (2) | 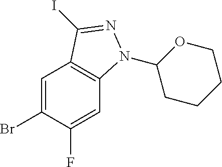 [SMILES: Fc1cc2c(cc1Br)c(I)nn2C1CCCCO1] | 5-Bromo-6-fluoro-3-iodo-1- (tetrahydro-2H-pyran-2-yl)-1H- indazole | 424.6 |
| 8.04 (1,3) | 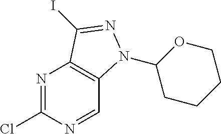 [SMILES: Clc1ncc2c(n1)c(I)nn2C1CCCCO1] | 5-Chloro-3-iodo-1-(tetrahydro-2H- pyran-2-yl)-1H-pyrazolo[4,3- d]pyrimidine | 364.8 |
| 8.05 (1) | 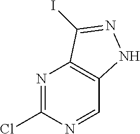 [SMILES: Clc1ncc2[nH]nc(I)c2n1] | 5-Chloro-3-iodo-1H-pyrazolo[4,3- d]pyrimidine | 280.7 |
| 8.06 (2) | 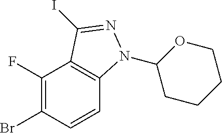 [SMILES: Fc1c(Br)ccc2c1c(I)nn2C1CCCCO1] | 5-Bromo-4-fluoro-3-iodo-1- (tetrahydro-2H-pyran-2-yl)-1H- indazole | 424.9 |
| 8.07 (2) | 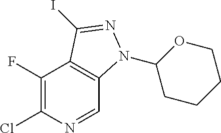 [SMILES: Fc1c(Cl)ncc2c1c(I)nn2C1CCCCO1] | 5-Chloro-4-fluoro-3-iodo-1- (tetrahydro-2H-pyran-2-yl)-1H- pyrazolo[3,4-c]pyridine | 381.9 |

Alternate conditions used: 1. Step 1: NIS, DMF, rt for 1-2 days or 80° C. for 4 h; 2. Step 1: NaOH or K₂CO₃ instead of KOH; 3. Step 2: Pyridinium p-toluenesulfonate, 3,4-dihydro-2H-pyran, THF, 60° C., 6 h.

### Intermediate 8.08

### 5-Chloro-3-iodo-1-((2-(trimethylsilyl)ethoxy)methyl)-1H-pyrazolo[4,3-d]pyrimidine

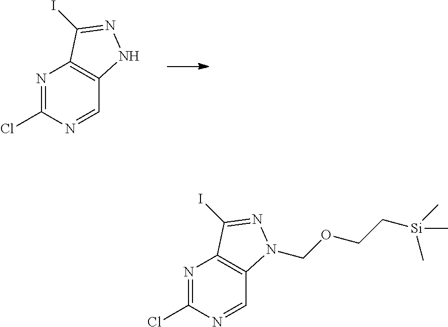 [SMILES: Clc1ncc2[nH]nc(I)c2n1 ; C[Si](C)(C)CCOCn1nc(I)c2nc(Cl)ncc21]

Sodium hydride (0.49 g, 12.8 mmol) was added slowly to a mixture of Intermediate 8.05 (2.00 g, 7.13 mmol) and SEM Cl (2.02 mL, 11.4 mmol) in THF (20 mL) at 0° C. The reaction was stirred at rt for 1 h. Additional SEM Cl (0.63 mL, 3.56 mmol) was added. The reaction was stirred overnight. Additional SEM Cl (0.38 mL, 2.14 mmol) was added. The reaction was stirred at rt for 90 min, quenched with water, and then extracted with ethyl acetate. The organic layer was washed with brine, dried (MgSO₄), concentrated, and then purified by column chromatography (0-40% ethyl acetate in heptane) to give 5-chloro-3-iodo-1-((2-(trimethylsilyl)ethoxy)methyl)-1H-pyrazolo[4,3-d]pyrimidine (2.35 g, 76%) as an off-white solid. ¹H NMR (400 MHz, DMSO-d₆) δ 9.54 (s, 1H), 5.88 (s, 2H), 3.58-3.53 (m, 2H), 0.83-0.78 (m, 2H), −0.08-−0.11 (m, 9H); LCMS: 411.2 [M+H]⁺.

The Intermediate below was synthesized in a similar manner to that described for Intermediate 8.08.

| Int | Structure | Name | [M + H]⁺ |
|---|---|---|---|
| 8.09 | 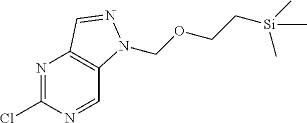 [SMILES: C[Si](C)(C)CCOCn1ncc2nc(Cl)ncc21] | 5-Chloro-1-((2- (trimethylsilyl)ethoxy)methyl)-1H- pyrazolo[4,3-d]pyrimidine | 284.9 |

### Intermediate 9

### 5-Bromo-6-(trifluoromethyl)-1H-indazole

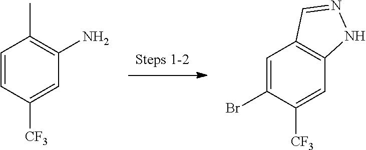

### Step 1: 4-Bromo-2-methyl-5-(trifluoromethyl)aniline

N-Bromosucccinimide (2.34 g, 13.1 mmol) was added to a solution of 2-methyl-5-(trifluoromethyl)aniline (2.0 g, 11.4 mmol) in acetonitrile (30 mL) at 10° C. The mixture was stirred at room temperature for 2 h, poured into water (50 mL), and then extracted (3×80 mL EtOAc). The combined organic layers were washed (2×50 mL brine), dried (Na₂SO₄), filtered, and then concentrated. The crude was purified by silica gel chromatography (1-4% EtOAc/petroleum ether) to give 4-bromo-2-methyl-5-(trifluoromethyl)aniline (1.8 g, 62%) as a yellow oil. ¹H NMR (400 MHz, CDCl₃): δ 7.34 (s, 1H), 6.96 (s, 1H), 3.77 (s, 2H), 2.17 (s, 3H); LCMS: 254.0 [M+H]⁺.

### Step 2: 5-Bromo-6-(trifluoromethyl)-1H-indazole

A solution of sodium nitrite (476 mg, 6.90 mmol) in water (1.7 mL) was added dropwise to a solution of 4-bromo-2-methyl-5-(trifluoromethyl)aniline (1.6 g, 6.30 mmol) in AcOH (61 mL) at room temperature. The mixture was stirred for 16 h, neutralized (pH>7) by the addition of saturated Na₂CO₃, and then extracted (3×120 mL EtOAc). The organic layers were washed (2×100 mL brine), dried (Na₂SO₄), filtered, and then concentrated. The crude reaction was purified by silica gel chromatography (2-10% EtOAc/petroleum ether) to give 5-bromo-6-(trifluoromethyl)-1H-indazole (1.5 g, 89%) as a yellow solid. ¹H NMR (400 MHz, DMSO-d₆): δ 13.67 (s, 1H), 8.33 (s, 1H), 8.21 (s, 1H), 8.07 (s, 1H); LCMS: 265.0 [M+H]⁺.

The Intermediate below was synthesized from 5-amino-2-bromo-4-methylbenzonitrile in a similar manner to that described for Intermediate 9.

| Int | Structure | Name | [M − H]⁻ |
|---|---|---|---|
| 9.01 | 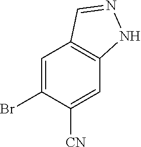 [SMILES: N#Cc1cc2[nH]ncc2cc1Br] | 5-Bromo-1H-indazole- 6-carbonitrile | 221.9 |

### Intermediate 10

### 5-(3-Chloro-4-methoxyphenyl)-1H-indazole

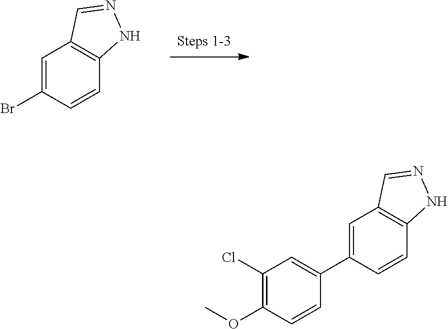 [SMILES: Brc1ccc2[nH]ncc2c1 ; COc1ccc(-c2ccc3[nH]ncc3c2)cc1Cl]

### Step 1: 5-Bromo-1-(tetrahydro-2H-pyran-2-yl)-1H-indazole

Pyridinium p-toluenesulfonate (535 mg, 2.13 mmol) was added to a mixture of 5-bromo-1H-indazole (4.18 g, 21.2 mmol), 3,4-dihydro-2H-pyran (10.0 mL, 109 mmol), and DCM (400 mL) at room temperature. The mixture was stirred overnight, diluted (100 mL DCM), washed (100 mL water and then 100 mL brine), dried (Na₂SO₄), and then concentrated. The residue was purified by silica gel chromatography eluting with 0-10% EtOAc/hexanes to give 5-bromo-1-(tetrahydro-2H-pyran-2-yl)-1H-indazole (6.13 g, >100%). ¹H NMR (400 MHz, DMSO-d₆): δ 8.10 (s, 1H), 8.03 (d, J=1.5 Hz, 1H), 7.73 (d, J=8.9 Hz, 1H), 7.54 (dd, J=1.9, 8.9 Hz, 1H), 5.86 (dd, J=2.4, 9.7 Hz, 1H), 3.92-3.84 (m, 1H), 3.78-3.69 (m, 1H), 2.46-2.30 (m, 1H), 2.07-1.92 (m, 2H), 1.80-1.67 (m, 1H), 1.62-1.54 (m, 2H).

### Step 2: 5-(4-Chloro-3-methoxyphenyl)-1-(tetrahydro-2H-pyran-2-yl)-1H-indazole

A mixture of 5-bromo-1-(tetrahydro-2H-pyran-2-yl)-1H-indazole (6.12 g, 21.8 mmol), 3-chloro-4-methoxyphenylboronic acid (6.10 g, 32.7 mmol), Pd(PPh₃)₄ (2.51 g, 2.17 mmol), Na₂CO₃ (2M, 22.0 mL, 44.0 mmol), and dioxane (40 mL) was degassed by bubbling N₂ through the suspension for 10 min, heated at 90° C. for 100 min, allowed to cool to room temperature, diluted (150 mL EtOAc), and then washed (100 mL water and then 100 mL brine). The organic layer was dried (Na₂SO₄), concentrated, and then purified by silica gel chromatography (0-20% EtOAc/hexanes) to give 5-(4-chloro-3-methoxyphenyl)-1-(tetrahydro-2H-pyran-2-yl)-1H-indazole (6.43 g, 85%) as a white foam. ¹H NMR (400 MHz, DMSO-d₆): δ 8.14 (s, 1H), 8.04-8.01 (m, 1H), 7.83-7.75 (m, 2H), 7.75-7.69 (m, 1H), 7.66 (dd, J=2.3, 8.6 Hz, 1H), 7.24 (d, J=8.7 Hz, 1H), 5.88 (dd, J=2.4, 9.7 Hz, 1H), 3.96-3.86 (m, 4H), 3.80-3.69 (m, 1H), 2.49-2.37 (m, 1H), 2.12-1.94 (m, 2H), 1.85-1.70 (m, 1H), 1.65-1.53 (m, 2H).

### Step 3: 5-(3-Chloro-4-methoxyphenyl)-1H-indazole

Hydrogen chloride (2 N in Et₂O, 60 mL, 120 mmol) was added to a mixture of 5-(4-chloro-3-methoxyphenyl)-1-(tetrahydro-2H-pyran-2-yl)-1H-indazole (6.42 g, 18.8 mmol) and methanol (60 mL) at room temperature. The mixture was stirred overnight and then filtered. The filter cake was rinsed with Et₂O (20 mL) to give 5-(3-chloro-4-methoxyphenyl)-1H-indazole (4.83 g, 100%) as a white solid. ¹H NMR (400 MHz, DMSO-d₆): δ 8.11 (d, J=0.9 Hz, 1H), 8.02-8.00 (m, 1H), 7.75 (d, J=2.3 Hz, 1H), 7.68-7.63 (m, 2H), 7.62-7.57 (m, 1H), 7.24 (d, J=8.7 Hz, 1H), 3.90 (s, 3H); LCMS 258.9 [M+H]⁺.

The Intermediates below were synthesized in a similar manner to that described for Intermediate 10.

| Int | Structure | Name | [M + H]⁺ |
|---|---|---|---|
| 10.01 | 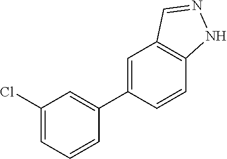 [SMILES: Clc1cccc(-c2ccc3[nH]ncc3c2)c1] | 5-(3-Chlorophenyl)-1H-indazole | 228.8 |
| 10.02 (1,2) | 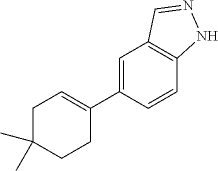 [SMILES: CC1(C)CC=C(c2ccc3[nH]ncc3c2)CC1] | 5-(4,4-Dimethylcyclohex-1-en-1-yl)- 1H-indazole | 226.9 |

Alternate conditions used: 1. Step 2: 1 M Na₂CO₃, Pd(dppf)Cl₂, CH₃CN, 80° C. (microwave); 2. Step 3: TFA:DCM (1:2), rt.

### Intermediate 11

### 5-(3-Chloro-4-methoxyphenyl)-1H-pyrazolo[4,3-b]pyridine

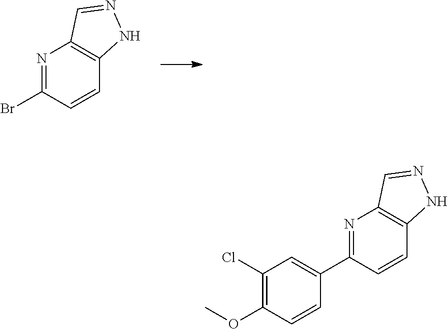 [SMILES: Brc1ccc2[nH]ncc2n1 ; COc1ccc(-c2ccc3[nH]ncc3n2)cc1Cl]

Pd(dppf)Cl₂ (0.05 g, 0.06 mmol) was added to a mixture of 5-bromo-1H-pyrazolo[4,3-b]pyridine (0.25 g, 1.26 mmol), 3-chloro-4-methoxyphenylboronic acid (0.28 g, 1.51 mmol), saturated Na₂CO₃ (1.50 mL), and acetonitrile (3 mL). The reaction mixture was irradiated in the microwave at 120° C. for 30 min, diluted (EtOAc), washed with water, and then washed with brine. The organics were dried (MgSO₄) and concentrated. The residue was purified by silica gel chromatography (0-50% EtOAc/heptane) to give 5-(3-chloro-4-methoxyphenyl)-1H-pyrazolo[4,3-b]pyridine (48 mg, 13%). LCMS 259.8 [M+H]⁺.

The Intermediates below were synthesized in a similar manner to that described for Intermediate 11.

| Int | Structure | Name | [M + H]⁺ |
|---|---|---|---|
| 11.01 | 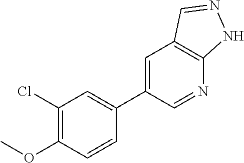 [SMILES: COc1ccc(-c2cnc3[nH]ncc3c2)cc1Cl] | 5-(3-Chloro-4-methoxyphenyl)-1H- pyrazolo[3,4-b]pyridine | 259.9 |
| 11.02 (1) | 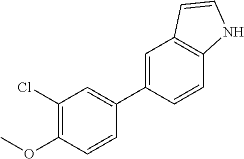 [SMILES: COc1ccc(-c2ccc3[nH]ccc3c2)cc1Cl] | 5-(3-Chloro-4-methoxyphenyl)-1H- indole | 258.0 |
| 11.03 | 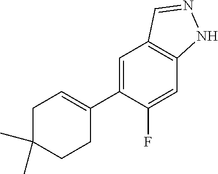 [SMILES: CC1(C)CC=C(c2cc3cn[nH]c3cc2F)CC1] | 5-(4,4-Dimethylcyclohex-1-en-1-yl)- 6-fluoro-1H-indazole | 245.2 |

Alternate conditions used: 1. Pd(PPh₃)₄, 1 M Na₂CO₃, dioxane, 100° C.

### Intermediate 12

### 5-(3-Chloro-4-((tetrahydro-2H-pyran-2-yl)oxy)phenyl)-1H-indazole

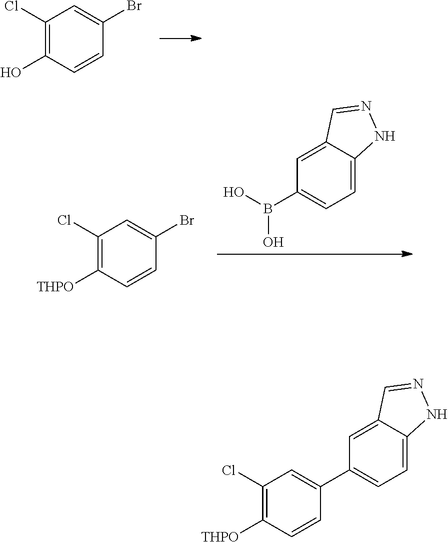

### Step 1: 2-(4-Bromo-2-chlorophenoxy)tetrahydro-2H-pyran

3,4-Dihydro-2H-pyran (25 mL, 274 mmol) was added over 2 min to a solution of 4-bromo-2-chlorophenol (20.8 g, 100 mmol), p-toluenesulfonic acid monohydrate (200 mg, 1.05 mmol), and THF (50 mL) at 0° C. under N₂ (exotherm from 3° C. to 7° C.). After 5 min the cooling bath was removed. After 2.5 h at room temperature, the reaction was poured into saturated aq. NaHCO₃ (250 mL) and extracted (250 mL EtOAc). The organic extract was washed (250 mL saturated aq. NaHCO₃), dried (MgSO₄), filtered, concentrated, and then purified by silica gel chromatography (0-15% EtOAc in heptane) to give 2-(4-bromo-2-chlorophenoxy)tetrahydro-2H-pyran (25.4 g) as a clear oil. ¹H NMR (400 MHz, DMSO-d₆): δ 7.69 (d, J=2.4 Hz, 1H), 7.47 (dd, J=2.4, 8.8 Hz, 1H), 7.21 (d, J=8.8 Hz, 1H), 5.64 (t, J 2.9 Hz, 1H), 3.74-3.65 (m, 1H), 3.59-3.52 (m, 1H), 1.96-1.84 (m, 1H), 1.84-1.77 (m, 2H), 1.69-1.51 (m, 3H).

### Step 2: 5-(3-Chloro-4-((tetrahydro-2H-pyran-2-yl)oxy)phenyl)-1H-indazole

A mixture of 2-(4-bromo-2-chlorophenoxy)tetrahydro-2H-pyran (1.76 g, 6.04 mmol), dioxane (15 mL), aq. K₃PO₄ (2 M, 9 mL, 18 mmol), and 1H-indazole-5-boronic acid (1.27 g, 7.84 mmol) was degassed with 2 vacuum/N₂ cycles. Pd(dppf)Cl₂ (242 mg, 0.331 mmol) was added. The mixture was again degassed with 2 vacuum/N₂ cycles, heated at 108° C. for 10 h, allowed to cool to room temperature, poured into saturated aq. NaHCO₃ (100 mL), and then extracted (2×100 mL EtOAc). The organic extracts were washed (100 mL saturated aq. NaHCO₃ solution), dried (MgSO₄), filtered, concentrated, and then purified by silica gel chromatography (10-40% EtOAc in heptane) to give 5-(3-chloro-4-((tetrahydro-2H-pyran-2-yl)oxy)phenyl)-1H-indazole (1.34 g, 67%) as an off-white solid. ¹H NMR (400 MHz, DMSO-d₆): δ 13.12 (s, 1H), 8.11 (s, 1H), 8.01 (s, 1H), 7.76 (d, J=2.3 Hz, 1H), 7.67-7.57 (m, 3H), 7.33 (d, J=8.7 Hz, 1H), 5.67 (t, J=2.9 Hz, 1H), 3.82-3.74 (m, 1H), 3.62-3.55 (m, 1H), 2.00-1.89 (m, 1H), 1.88-1.82 (m, 2H), 1.71-1.54 (m, 3H); LCMS: 329.0 [M+H]⁺.

### Intermediate 13

### 6-Chloro-5-(4-(methylsulfonyl)piperazin-1-yl)-1H-indazole

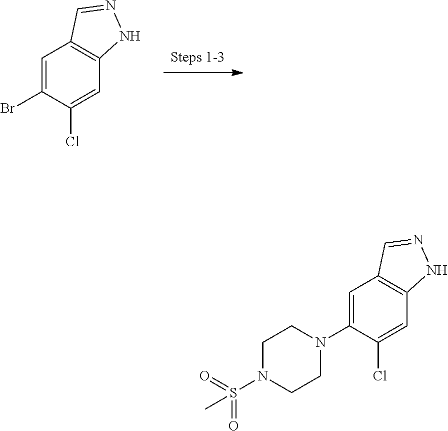 [SMILES: Clc1cc2[nH]ncc2cc1Br ; CS(=O)(=O)N1CCN(c2cc3cn[nH]c3cc2Cl)CC1]

### Step 1: 5-Bromo-6-chloro-1-(tetrahydro-2H-pyran-2-yl)-1H-indazole

p-Toluenesulfonic acid (0.080 g, 0.43 mmol) was added to a suspension of 5-bromo-6-chloro-1H-indazole (1.00 g, 4.32 mmol) and 2H-3,4-dihydropyran (0.59 mL, 6.48 mmol) in DCM (10 mL) at room temperature. The reaction was stirred overnight and quenched by the addition of saturated NaHCO₃. The phases were separated, and the aqueous layer was extracted with DCM. The combined organic phases were dried (MgSO₄) and concentrated. The residue was purified by silica gel chromatography (0-25% EtOAc/heptane). The resulting solid was triturated in acetonitrile to provide 5-bromo-6-chloro-1-(tetrahydro-2H-pyran-2-yl)-1H-indazole (0.95 g, 66%) as an off-white solid. ¹H NMR (400 MHz, DMSO-d₆): δ 8.29-8.23 (m, 1H), 8.19-8.12 (m, 2H), 5.93-5.85 (m, 1H), 3.92-3.83 (m, 1H), 3.82-3.72 (m, 1H), 2.42-2.27 (m, 1H), 2.11-1.92 (m, 2H), 1.80-1.67 (m, 1H), 1.63-1.51 (m, 2H); LCMS: 230.8 [(M−THP+H)+H]⁺.

### Step 2: 6-Chloro-5-(4-(methylsulfonyl)piperazin-1-yl)-1-(tetrahydro-2H-pyran-2-yl)-1H-indazole

Pd₂(dba)₃ (64 mg, 0.07 mmol) was added to a mixture of 5-bromo-6-chloro-1-(tetrahydro-2H-pyran-2-yl)-1H-indazole (440 mg, 1.39 mmol), 1-methanesulfonyl-piperazine (275 mg, 1.67 mmol), BINAP (87 mg, 0.14 mmol), and Cs₂CO₃ (681 mg, 2.09 mmol) in toluene (5 mL). The reaction mixture was heated at 100° C. for 48 h, diluted (water), and then extracted (EtOAc). The organics were dried (MgSO₄) and concentrated. The residue was purified by silica gel chromatography (0-40% EtOAc/heptane) to provide 6-chloro-5-(4-(methylsulfonyl)piperazin-1-yl)-1-(tetrahydro-2H-pyran-2-yl)-1H-indazole (210 mg, 36%) as a yellow solid. ¹H NMR (400 MHz, DMSO-d₆): δ 8.07 (s, 1H), 7.94 (s, 1H), 7.58 (s, 1H), 5.84 (dd, J=2.4, 9.6 Hz, 1H), 3.90-3.81 (m, 1H), 3.80-3.71 (m, 1H), 3.32-3.24 (m, 4H), 3.11-3.02 (m, 4H), 2.99-2.94 (m, 3H), 2.41-2.31 (m, 1H), 2.07-2.00 (m, 1H), 1.98-1.91 (m, 1H), 1.80-1.67 (m, 1H), 1.63-1.53 (m, 2H); LCMS 399.0 [M+H]⁺.

### Step 3: 6-Chloro-5-(4-(methylsulfonyl)piperazin-1-yl)-1H-indazole

A solution of 6-chloro-5-(4-(methylsulfonyl)piperazin-1-yl)-1-(tetrahydro-2H-pyran-2-yl)-1H-indazole (295 mg, 0.74 mmol) in DCM (9 mL) and TFA (3 mL) was stirred at room temperature for 3 h and then concentrated. The residue was dissolved in EtOAc, washed (NaHCO₃ and then brine), dried (MgSO₄), and then concentrated. The residue was triturated in DCM, sonicated, and then filtered. The filter cake was washed with heptane to provide 6-chloro-5-(4-(methylsulfonyl)piperazin-1-yl)-1H-indazole (170 mg, 69%) as an off-white solid. ¹H NMR (400 MHz, DMSO-d₆): δ 13.16-12.95 (m, 1H), 8.04-8.00 (m, 1H), 7.67 (s, 1H), 7.56 (s, 1H), 3.32-3.26 (m, 4H), 3.04 (br s, 4H), 2.96 (s, 3H); LCMS 314.9 [M+H]⁺.

The Intermediates below were synthesized in a similar manner to that described for Intermediate 13.

| Int | Structure | Name | [M + H]⁺ |
|---|---|---|---|
| 13.01 | 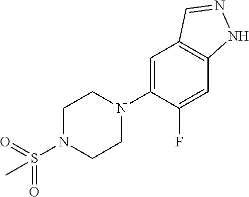 [SMILES: CS(=O)(=O)N1CCN(c2cc3cn[nH]c3cc2F)CC1] | 6-Fluoro-5-(4- (methylsulfonyl)piperazin-1-yl)-1H- indazole | 299.0 |
| 13.02 (1) | 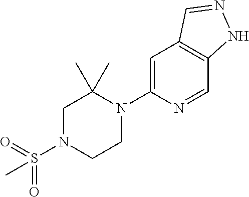 [SMILES: CC1(C)CN(S(C)(=O)=O)CCN1c1cc2cn[nH]c2cn1] | 5-(2,2-Dimethyl-4- (methylsulfonyl)piperazin-1-yl)-1H- pyrazolo[3,4-c]pyridine | 309.9 |
| 13.03 (1) | 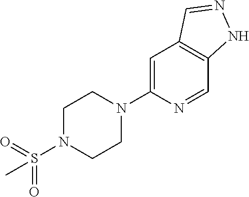 [SMILES: CS(=O)(=O)N1CCN(c2cc3cn[nH]c3cn2)CC1] | 5-(4-(Methylsulfonyl)piperazin-1-yl)- 1H-pyrazolo[3,4-c]pyridine | 281.9 |
| 13.04 (1) | 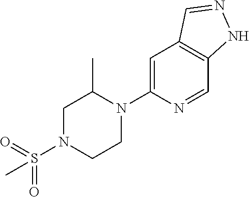 [SMILES: CC1CN(S(C)(=O)=O)CCN1c1cc2cn[nH]c2cn1] | 5-(2-Methyl-4- (methylsulfonyl)piperazin-1-yl)-1H- pyrazolo[3,4-c]pyridine | 295.9 |
| 13.05 (1) | 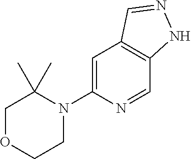 [SMILES: CC1(C)COCCN1c1cc2cn[nH]c2cn1] | 3,3-Dimethyl-4-(1H-pyrazolo[3,4- c]pyridine-5-yl)morpholine | 232.9 |
| 13.06 | 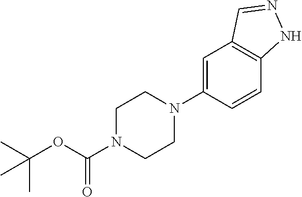 [SMILES: CC(C)(C)OC(=O)N1CCN(c2ccc3[nH]ncc3c2)CC1] | tert-Butyl 4-(1H-indazol-5- yl)piperazine-1-carboxylate | 303.2 |
| 13.07 (6) | 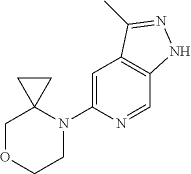 [SMILES: Cc1n[nH]c2cnc(N3CCOCC34CC4)cc12] | 4-(3-Methyl-1H-pyrazolo[3,4- c]pyridin-5-yl)-7-oxa-4- azaspiro[2.5]octane | 245.0 |
| 13.08 (2,6) | 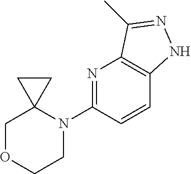 [SMILES: Cc1n[nH]c2ccc(N3CCOCC34CC4)nc12] | 4-(3-Methyl-1H-pyrazolo[4,3- b]pyridin-5-yl)-7-oxa-4- azaspiro[2.5]octane | 245.2 |
| 13.09 (2) | 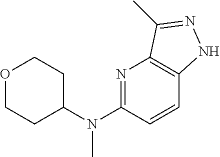 [SMILES: Cc1n[nH]c2ccc(N(C)C3CCOCC3)nc12] | N,3-Dimethyl-N-(tetrahydro-2H- pyran-4-yl)-1H-pyrazolo[4,3- b]pyridin-5-amine | 247.1 |
| 13.10 (3) | 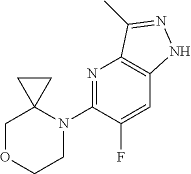 [SMILES: Cc1n[nH]c2cc(F)c(N3CCOCC34CC4)nc12] | 4-(6-Fluoro-3-methyl-1H- pyrazolo[4,3-b]pyridin-5-yl)-7-oxa-4- azaspiro[2.5]octane | 263.1 |
| 13.11 | 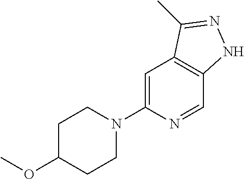 [SMILES: COC1CCN(c2cc3c(C)n[nH]c3cn2)CC1] | 5-(4-Methoxypiperidin-1-yl)-3- methyl-1H-pyrazolo[3,4-c]pyridine | 246.9 |
| 13.12 | 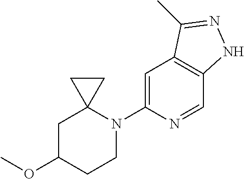 [SMILES: COC1CCN(c2cc3c(C)n[nH]c3cn2)C2(CC2)C1] | 5-(7-Methoxy-4-azaspiro[2.5]octan- 4-yl)-3-methyl-1H-pyrazolo[3,4- c]pyridine | 273.0 |
| 13.13 | 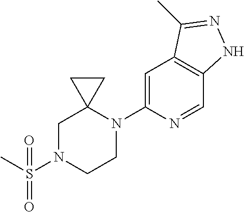 [SMILES: Cc1n[nH]c2cnc(N3CCN(S(C)(=O)=O)CC34CC4)cc12] | 3-Methyl-5-(7-(methylsulfonyl)-4,7- diazaspiro[2.5]octan-4-yl)-1H- pyrazolo[3,4-c]pyridine | 322.0 |
| 13.14 | 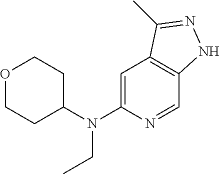 [SMILES: CCN(c1cc2c(C)n[nH]c2cn1)C1CCOCC1] | N-Ethyl-3-methyl-N-(tetrahydro-2H- pyran-4-yl)-1H-pyrazolo[3,4- c]pyridin-5-amine | 261.0 |
| 13.15 | 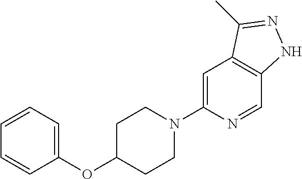 [SMILES: Cc1n[nH]c2cnc(N3CCC(Oc4ccccc4)CC3)cc12] | 3-Methyl-5-(4-phenoxypiperidin-1- yl)-1H-pyrazolo[3,4-c]pyridine | 309.0 |
| 13.16 | 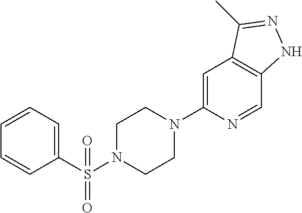 [SMILES: Cc1n[nH]c2cnc(N3CCN(S(=O)(=O)c4ccccc4)CC3)cc12] | 3-Methyl-5-(4- (phenylsulfonyl)piperazin-1-yl)-1H- pyrazolo[3,4-c]pyridine | 358.0 |
| 13.17 | 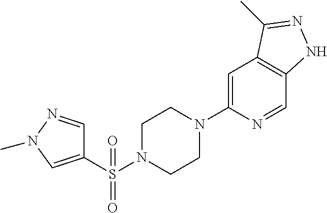 [SMILES: Cc1n[nH]c2cnc(N3CCN(S(=O)(=O)c4cnn(C)c4)CC3)cc12] | 3-Methyl-5-(4-((1-methyl-1H- pyrazol-4-yl)sulfonyl)piperazin-1-yl)- 1H-pyrazolo[3,4-c]pyridine | 362.0 |
| 13.18 | 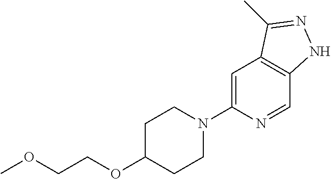 [SMILES: COCCOC1CCN(c2cc3c(C)n[nH]c3cn2)CC1] | 5-(4-(2-Methoxyethoxy)piperidin-1- yl)-3-methyl-1H-pyrazolo[3,4- c]pyridine | 291.0 |
| 13.19 | 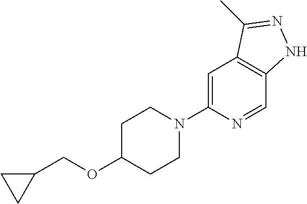 [SMILES: Cc1n[nH]c2cnc(N3CCC(OCC4CC4)CC3)cc12] | 5-(4-(Cyclopropylmethoxy)piperidin- 1-yl)-3-methyl-1H-pyrazolo[3,4- c]pyridine | 287.0 |
| 13.20 | 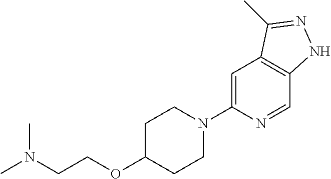 [SMILES: Cc1n[nH]c2cnc(N3CCC(OCCN(C)C)CC3)cc12] | N,N-Dimethyl-2-((1-(3-methyl-1H- pyrazolo[3,4-c]pyridin-5- yl)piperidin-4-yl)oxy)ethanamine | 304.0 |
| 13.21 | 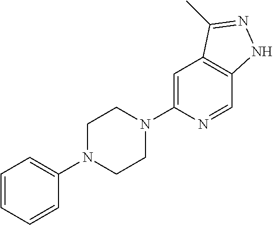 [SMILES: Cc1n[nH]c2cnc(N3CCN(c4ccccc4)CC3)cc12] | 3-Methyl-5-(4-phenylpiperazin-1-yl)- 1H-pyrazolo[3,4-c]pyridine | 294.3 |
| 13.22 | 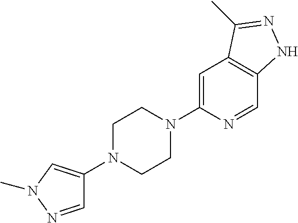 [SMILES: Cc1n[nH]c2cnc(N3CCN(c4cnn(C)c4)CC3)cc12] | 3-Methyl-5-(4-(1-methyl-1H-pyrazol- 4-yl)piperazin-1-yl)-1H- pyrazolo[3,4-c]pyridine | 298.3 |
| 13.23 (6) | 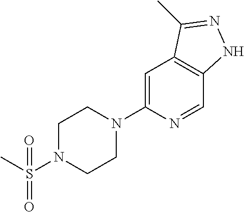 [SMILES: Cc1n[nH]c2cnc(N3CCN(S(C)(=O)=O)CC3)cc12] | 3-Methyl-5-(4- (methylsulfonyl)piperazin-1-yl)-1H- pyrazolo[3,4-c]pyridine | 296.0 |
| 13.24 | 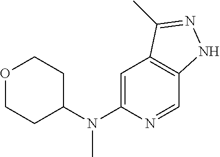 [SMILES: Cc1n[nH]c2cnc(N(C)C3CCOCC3)cc12] | N,3-Dimethyl-N-(tetrahydro-2H- pyran-4-yl)-1H-pyrazolo[3,4- c]pyridin-5-amine | 247.0 |
| 13.25 (6) | 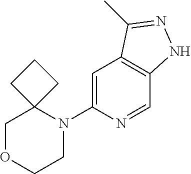 [SMILES: Cc1n[nH]c2cnc(N3CCOCC34CCC4)cc12] | 5-(3-Methyl-1H-pyrazolo[3,4- c]pyridin-5-yl)-8-oxa-5- azaspiro[3.5]nonane | 259.2 |
| 13.26 (3,6) | 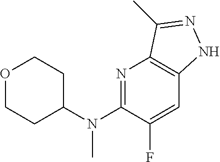 [SMILES: Cc1n[nH]c2cc(F)c(N(C)C3CCOCC3)nc12] | 6-Fluoro-N,3-dimethyl-N- (tetrahydro-2H-pyran-4-yl)-1H- pyrazolo[4,3-b]pyridin-5-amine | 265.1 |
| 13.27 (7) | 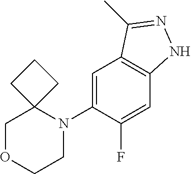 [SMILES: Cc1n[nH]c2cc(F)c(N3CCOCC34CCC4)cc12] | 4-(6-Fluoro-3-methyl-1H-indazol-5- yl)-7-oxa-4-azaspiro[2.5]octane | 262.2 |
| 13.28 (7) | 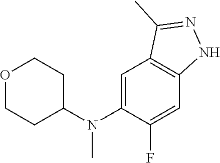 [SMILES: Cc1n[nH]c2cc(F)c(N(C)C3CCOCC3)cc12] | 6-Fluoro-N,3-dimethyl-N- (tetrahydro-2H-pyran-4-yl)-1H- indazol-5-amine | 264.1 |
| 13.29 (6) | 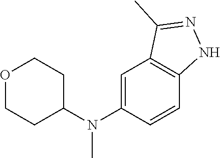 [SMILES: Cc1n[nH]c2ccc(N(C)C3CCOCC3)cc12] | N,3-Dimethyl-N-(tetrahydro-2H- pyran-4-yl)-1H-indazol-5-amine | 246.3 |
| 13.30 (6) | 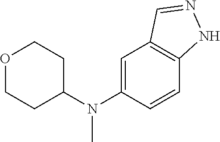 [SMILES: CN(c1ccc2[nH]ncc2c1)C1CCOCC1] | N-Methyl-N-(tetrahydro-2H-pyran-4- yl)-1H-indazol-5-amine | 232.2 |
| 13.31 (6) | 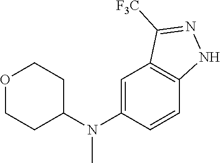 [SMILES: CN(c1ccc2[nH]nc(C(F)(F)F)c2c1)C1CCOCC1] | N-Methyl-N-(tetrahydro-2H-pyran-4- yl)-3-(trifluoromethyl)-1H-indazol-5- amine | 300.3 |
| 13.32 (6) | 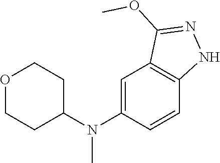 [SMILES: COc1n[nH]c2ccc(N(C)C3CCOCC3)cc12] | 3-Methoxy-N-methyl-N-(tetrahydro- 2H-pyran-4-yl)-1H-indazol-5-amine | 262.2 |
| 13.33 (6) | 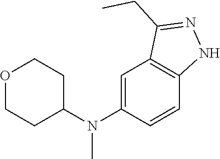 [SMILES: CCc1n[nH]c2ccc(N(C)C3CCOCC3)cc12] | 3-Ethyl-N-methyl-N-(tetrahydro-2H- pyran-4-yl)-1H-indazol-5-amine | 260.4 |
| 13.34 (6) | 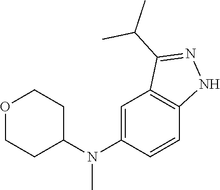 [SMILES: CC(C)c1n[nH]c2ccc(N(C)C3CCOCC3)cc12] | 3-Isopropyl-N-methyl-N-(tetrahydro- 2H-pyran-4-yl)-1H-indazol-5-amine | 274.2 |
| 13.35 (6) | 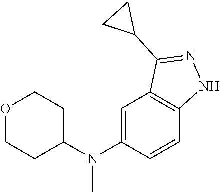 [SMILES: CN(c1ccc2[nH]nc(C3CC3)c2c1)C1CCOCC1] | 3-Cyclopropyl-N-methyl-N- (tetrahydro-2H-pyran-4-yl)-1H- indazol-5-amine | 272.2 |
| 13.36 (6) | 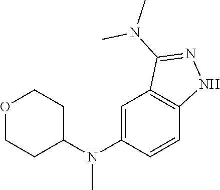 [SMILES: CN(C)c1n[nH]c2ccc(N(C)C3CCOCC3)cc12] | N³,N³,N⁵-trimethyl-N⁵-(tetrahydro- 2H-pyran-4-yl)-1H-indazole-3,5- diamine | 275.2 |
| 13.37 (4,8) | 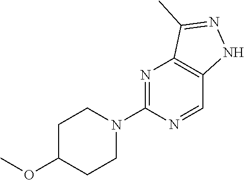 [SMILES: COC1CCN(c2ncc3[nH]nc(C)c3n2)CC1] | 5-(4-Methoxypiperidin-1-yl)-3- methyl-1H-pyrazolo[4,3- d]pyrimidine | 248.1 |
| 13.38 (4,8) | 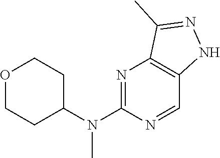 [SMILES: Cc1n[nH]c2cnc(N(C)C3CCOCC3)nc12] | N,3-Dimethyl-N-(tetrahydro-2H- pyran-4-yl)-1H-pyrazolo[4,3- d]pyrimidin-5-amine | 248.1 |
| 13.39 (5,8) | 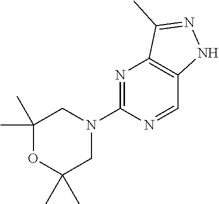 [SMILES: Cc1n[nH]c2cnc(N3CC(C)(C)OC(C)(C)C3)nc12] | 2,2,6,6-Tetramethyl-4-(3-methyl-1H- pyrazolo[4,3-d]pyrimidin-5- yl)morpholine | 276.0 |
| 13.40 (6,9) | 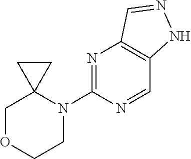 [SMILES: c1n[nH]c2cnc(N3CCOCC34CC4)nc12] | 4-(1H-Pyrazolo[4,3-d]pyrimidin-5- yl)-7-oxa-4-azaspiro[2.5]octane | 232.0 |
| 13.41 (5,8) | 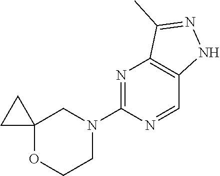 [SMILES: Cc1n[nH]c2cnc(N3CCOC4(CC4)C3)nc12] | 7-(3-Methyl-1H-pyrazolo[4,3- d]pyrimidin-5-yl)-4-oxa-7- azaspiro[2.5]octane | 245.9 |
| 13.42 (5,8) | 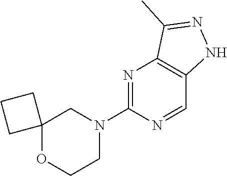 [SMILES: Cc1n[nH]c2cnc(N3CCOC4(CCC4)C3)nc12] | 8-(3-Methyl-1H-pyrazolo[4,3- d]pyrimidin-5-yl)-5-oxa-8- azaspiro[3.5]nonane | 260.0 |
| 13.43 (6) | 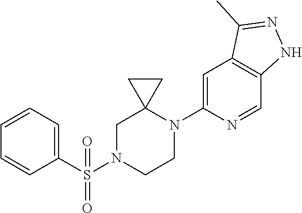 [SMILES: Cc1n[nH]c2cnc(N3CCN(S(=O)(=O)c4ccccc4)CC34CC4)cc12] | 3-Methyl-5-(7-(phenylsulfonyl)-4,7- diazaspiro[2.5]octan-4-yl)-1H- pyrazolo[3,4-c]pyridine | 384.4 |
| 13.44 (6) | 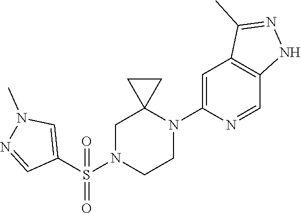 [SMILES: Cc1n[nH]c2cnc(N3CCN(S(=O)(=O)c4cnn(C)c4)CC34CC4)cc12] | 3-Methyl-5-(7-((1-methyl-1H- pyrazol-4-yl)sulfonyl)-4,7- diazaspiro[2.5]octan-4-yl)-1H- pyrazolo[3,4-c]pyridine | 388.5 |
| 13.45 (6) | 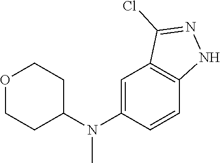 [SMILES: CN(c1ccc2[nH]nc(Cl)c2c1)C1CCOCC1] | 3-Chloro-N-methyl-N-(tetrahydro- 2H-pyran-4-yl)-1H-indazol-5-amine | 266.1 |
| 13.46 (6,10) | 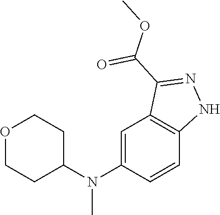 [SMILES: COC(=O)c1n[nH]c2ccc(N(C)C3CCOCC3)cc12] | Methyl 5-(methyl(tetrahydro-2H- pyran-4-yl)amino)-1H-indazole-3- carboxylate | 290.3 |
| 13.47 | 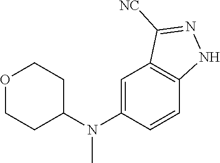 [SMILES: CN(c1ccc2[nH]nc(C#N)c2c1)C1CCOCC1] | 5-(Methyl(tetrahydro-2H-pyran-4- yl)amino)-1H-indazole-3-carbonitrile | 257.1 |
| 13.48 (11) | 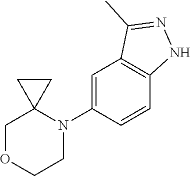 [SMILES: Cc1n[nH]c2ccc(N3CCOCC34CC4)cc12] | 4-(3-Methyl-1H-indazol-5-yl)-7-oxa- 4-azaspiro[2.5]octane | 244.2 |
| 13.49 (5,6) | 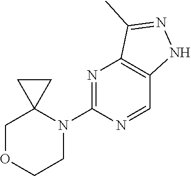 [SMILES: Cc1n[nH]c2cnc(N3CCOCC34CC4)nc12] | 4-(3-Methyl-1H-pyrazolo[4,3- d]pyrimidin-5-yl)-7-oxa-4- azaspiro[2.5]octane | 246.2 |
| 13.50 (7) | 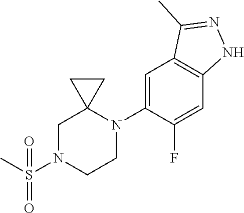 [SMILES: Cc1n[nH]c2cc(F)c(N3CCN(S(C)(=O)=O)CC34CC4)cc12] | 6-Fluoro-3-methyl-5-(7- (methylsulfonyl)-4,7- diazaspiro[2.5]octan-4-yl)-1H- indazole | 339.2 |
| 13.51 (6,12) | 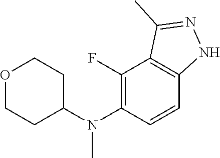 [SMILES: Cc1n[nH]c2ccc(N(C)C3CCOCC3)c(F)c12] | 4-Fluoro-N,3-dimethyl-N- (tetrahydro-2H-pyran-4-yl)-1H- indazol-5-amine | 264.2 |
| 13.52 (6,12) | 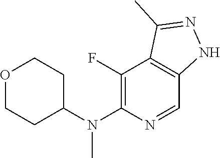 [SMILES: Cc1n[nH]c2cnc(N(C)C3CCOCC3)c(F)c12] | 4-Fluoro-N,3-dimethyl-N- (tetrahydro-2H-pyran-4-yl)-1H- pyrazolo[3,4-c]pyridin-5-amine | 265.2 |
| 13.53 (6) | 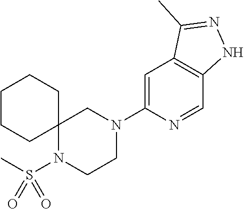 [SMILES: Cc1n[nH]c2cnc(N3CCN(S(C)(=O)=O)C4(CCCCC4)C3)cc12] | 3-Methyl-5-(1-(methylsulfonyl)-1,4- diazaspiro[5.5]undecan-4-yl)-1H- pyrazolo[3,4-c]pyridine | 364.6 |
| 13.54 (5,6) | 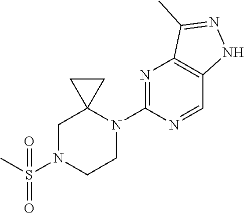 [SMILES: Cc1n[nH]c2cnc(N3CCN(S(C)(=O)=O)CC34CC4)nc12] | 3-Methyl-5-(7-(methylsulfonyl)-4,7- diazaspiro[2.5]octan-4-yl)-1H- pyrazolo[4,3-d]pyrimidine | 323.3 |
| 13.55 (6) | 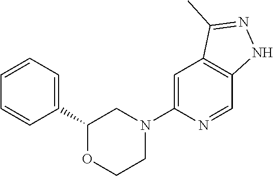 [SMILES: Cc1n[nH]c2cnc(N3CCO[C@H](c4ccccc4)C3)cc12] | (R)-4-(3-Methyl-1H-pyrazolo[3,4- c]pyridin-5-yl)-2-phenylmorpholine | 295.4 |
| 13.56 (6) | 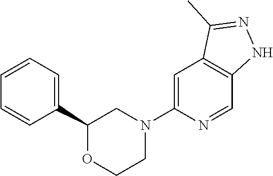 [SMILES: Cc1n[nH]c2cnc(N3CCO[C@@H](c4ccccc4)C3)cc12] | (S)-4-(3-Methyl-1H-pyrazolo[3,4- c]pyridin-5-yl)-2-phenylmorpholine | 295.3 |
| 13.57 (2,6,13) | 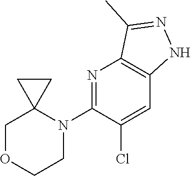 [SMILES: Cc1n[nH]c2cc(Cl)c(N3CCOCC34CC4)nc12] | 4-(6-Chloro-3-methyl-1H- pyrazolo[4,3-b]pyridin-5-yl)-7-oxa-4- azaspiro[2.5]octane | 279.1 |
| 13.58 (2,6,13) | 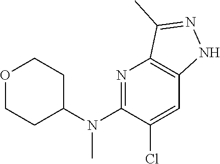 [SMILES: Cc1n[nH]c2cc(Cl)c(N(C)C3CCOCC3)nc12] | 6-Chloro-N,3-dimethyl-N- (tetrahydro-2H-pyran-4-yl)-1H- pyrazolo[4,3-b]pyridin-5-amine | 281.1 |
| 13.59 (5,14) | 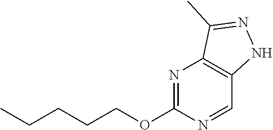 [SMILES: CCCCCOc1ncc2[nH]nc(C)c2n1] | 3-Methyl-5-(pentyloxy)-1H- pyrazolo[4,3-d]pyrimidine | 221.4 |
| 13.60 (5,8) | 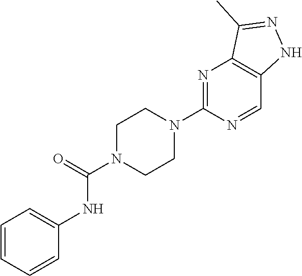 [SMILES: Cc1n[nH]c2cnc(N3CCN(C(=O)Nc4ccccc4)CC3)nc12] | 4-(3-Methyl-1H-pyrazolo[4,3- d]pyrimidin-5-yl)-N- phenylpiperazine-1-carboxamide | 338.5 |
| 13.61 (6,15) | 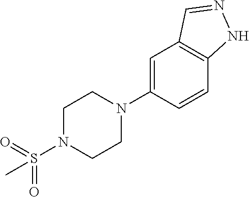 [SMILES: CS(=O)(=O)N1CCN(c2ccc3[nH]ncc3c2)CC1] | 5-(4-(Methylsulfonyl)piperazin-1-yl)- 1H-indazole | 281.1 |

Alternate conditions used: Step 1: toluene was used instead of DCM; Step 1: rt or 70° C.; In some instances, only TFA was used to deprotect THP in Step 3. In some instances, molecular sieves 4A were used in Step 3. 1. Used aryl chloride; 2. From Intermediate 24.02; 3. From Intermediate 24.03; 4. From Intermediate 24.05; 5. From Intermediate 24.06; 6. Step 2: Pd₂(dba)₃, RuPhos, NaO^{t}Bu, dioxane, 80-100° C., 30 min-overnight; 7. Step 2: t-BuXPhos Pd G3, NaO^{t}Bu, dioxane, 50° C. or 90° C., overnight; 8. Step 2: DIEA, NMP or DMA, 100-150° C., overnight; 9. From Intermediate 8.09; 10. Step 2 only from methyl 5-bromo-1H-indazole-3-carboxylate; 11. Step 2: 2-[Bis(3,5-trifluoromethylphenylphosphino)-3,6-dimethoxy]-2′,6′-dimethylamino-1,1′-biphenyl, methanesulfonato(2-bis(3,5-di(trifluoromethyl)phenylphosphino)-3,6-dimethoxy-2′,6′-bis(dimethylamino)-1,1′-biphenyl)(2′-methylamino-1,1′-biphenyl-2-yl)palladium(II), NaO^{t}Bu, CPME, 60° C., ON; 12. Synthesized from Intermediate 24.07 or 24.08 using the following sequence: Step 2, methylation (K₂CO₃, Mel, DMF, rt, ON), and then Step 3; 13. Step 2, chlorination (NCS, MeCN, 80° C., 2 h), and then Step 3; 14. Step 2: DBU, pentanol, 140° C., 4 h; 15. Step 3: 4 M HCl in EtOAc, rt, 2 h.

### Intermediate 14

### 5-Bromo-1-(4-fluoro-3-methoxyphenyl)-1H-indazole

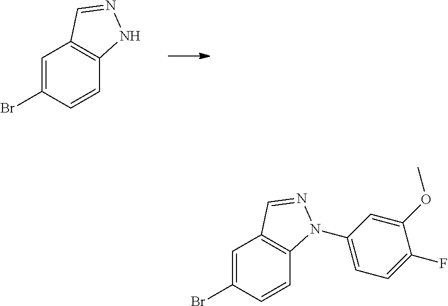 [SMILES: Brc1ccc2[nH]ncc2c1 ; COc1cc(-n2ncc3cc(Br)ccc32)ccc1F]

Copper acetate (11.1 g, 61.0 mmol) was added to a mixture of 5-bromo-1H-indazole (5.99 g 30.4 mmol), 4-fluoro-3-methoxyphenylboronic acid (7.79 g, 45.8 mmol), pyridine (5.0 mL, 61.8 mmol), and DCM (300 mL) at room temperature. The mixture was stirred for 19 h and then filtered through a Celite plug. The filter cake was washed (˜50 mL DCM) and the filtrate was concentrated. The residue was purified by silica gel chromatography (0-10% EtOAc/hexanes) to give 5-bromo-1-(4-fluoro-3-methoxyphenyl)-1H-indazole (3.17 g, 32%) as a white solid. ¹H NMR (400 MHz, DMSO-d₆): δ 8.36 (d, J=0.7 Hz, 1H), 8.15 (d, J=1.5 Hz, 1H), 7.81 (d, J=8.9 Hz, 1H), 7.60 (dd, J=1.9, 9.0 Hz, 1H), 7.49 (dd, J=2.6, 7.7 Hz, 1H), 7.44 (dd, J=8.7, 11.1 Hz, 1H), 7.30 (ddd, J=2.6, 3.9, 8.7 Hz, 1H), 3.94 (s, 3H); LCMS: 320.8 [M+H]⁺.

The Intermediates below were synthesized in a similar manner to that described for Intermediate 14.

| Int | Structure | Name | [M + H]⁺ |
|---|---|---|---|
| 14.01 | 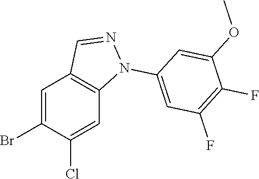 [SMILES: COc1cc(-n2ncc3cc(Br)c(Cl)cc32)cc(F)c1F] | 5-Bromo-6-chloro-1-(3,4- difluoro-5-methoxyphenyl)-1H- indazole | 374.7 |
| 14.02 | 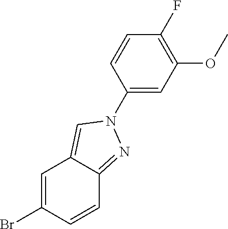 [SMILES: COc1cc(-n2cc3cc(Br)ccc3n2)ccc1F] | 5-Bromo-2-(4-fluoro-3- methoxyphenyl)-2H-indazole | 320.8 |
| 14.03 | 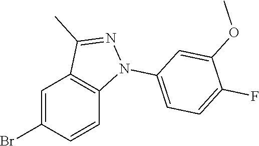 [SMILES: COc1cc(-n2nc(C)c3cc(Br)ccc32)ccc1F] | 5-Bromo-1-(4-fluoro-3- methoxyphenyl)-3-methyl-1H- indazole | ¹H NMR (1) |
| 14.04 | 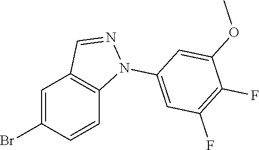 [SMILES: COc1cc(-n2ncc3cc(Br)ccc32)cc(F)c1F] | 5-Bromo-1-(3,4-difluoro-5- methoxyphenyl)-1H-indazole | 338.9 |
| 14.05 | 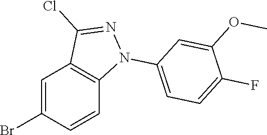 [SMILES: COc1cc(-n2nc(Cl)c3cc(Br)ccc32)ccc1F] | 5-Bromo-3-chloro-1-(4-fluoro-3- methoxyphenyl)-1H-indazole | 354.9 |
| 14.06 | 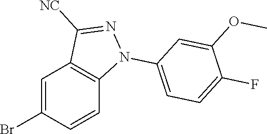 [SMILES: COc1cc(-n2nc(C#N)c3cc(Br)ccc32)ccc1F] | 5-Bromo-1-(4-fluoro-3- methoxyphenyl)-1H-indazole-3- carbonitrile | 345.9 |
| 14.07 | 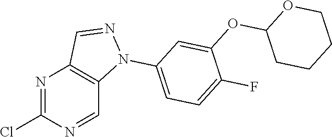 [SMILES: Fc1ccc(-n2ncc3nc(Cl)ncc32)cc1OC1CCCCO1] | 5-Chloro-1-(4-fluoro-3- ((tetrahydro-2H-pyran-2- yl)oxy)phenyl)-1H-pyrazolo[4,3- d]pyrimidine | 264.8 [(M − THP + H) + H]⁺ |
| 14.08 | 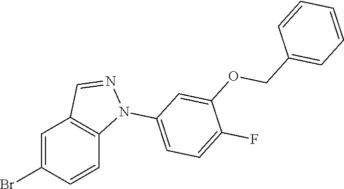 [SMILES: Fc1ccc(-n2ncc3cc(Br)ccc32)cc1OCc1ccccc1] | 1-(3-(Benzyloxy)-4- fluorophenyl)-5-bromo-1H- indazole | 396.9 |
| 14.09 | 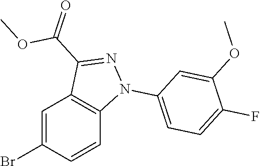 [SMILES: COC(=O)c1nn(-c2ccc(F)c(OC)c2)c2ccc(Br)cc12] | Methyl 5-bromo-1-(4-fluoro-3- methoxyphenyl)-1H-indazole-3- carboxylate | 379.0 |
| 14.10 | 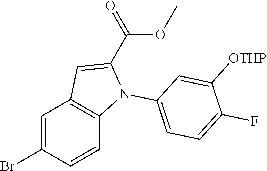 | Methyl 5-bromo-1-(4-fluoro-3- ((tetrahydro-2H-pyran-2- yl)oxy)phenyl)-1H-indole-2- carboxylate | 362.1 [M − THP]⁺ |

1. Intermediate 14.03: ¹H NMR (400 MHz, DMSO-d₆): δ 8.11 (s, 1H), 7.76-7.74 (m, 1H), 7.58-7.56 (m, 1H), 7.44-7.40 (m, 2H), 7.36-7.35 (m, 1H), 3.93 (s, 3H), 2.57 (s, 3H).

### Intermediate 15

### 5-(5-Bromo-1H-indazol-1-yl)-2-fluorophenol

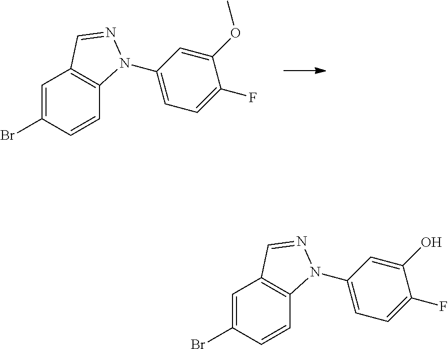 [SMILES: COc1cc(-n2ncc3cc(Br)ccc32)ccc1F ; Oc1cc(-n2ncc3cc(Br)ccc32)ccc1F]

Boron tribromide (3.8 mL, 40 mmol) was added dropwise to a solution of Intermediate 14 (3.19 g, 9.93 mmol) in DCM (45 mL) precooled in a dry ice/acetone bath. The mixture was stirred at that temperature for 5 min, placed in refrigerator overnight, stirred at room temperature for 2 h, and then re-cooled in a dry ice/acetone bath. Methanol (20 mL) was slowly added. The reaction was allowed to warm room temperature and concentrated. The resulting solid was triturated in methanol (30 mL) and dried under reduced pressure to give 5-(5-bromo-1H-indazol-1-yl)-2-fluorophenol (2.84 g, 93%) as a beige solid. ¹H NMR (400 MHz, DMSO-d₆): δ 10.35 (br dd, J=2.2, 4.9 Hz, 1H), 8.34 (d, J=0.7 Hz, 1H), 8.14 (d, J=1.5 Hz, 1H), 7.75 (d, J=8.9 Hz, 1H), 7.61 (dd, J=1.9, 9.0 Hz, 1H), 7.39-7.30 (m, 2H), 7.19-7.13 (m, 1H); LCMS 306.8 [M+H]⁺.

### Intermediate 16

### 5-Bromo-1-(3-((tert-butyldimethylsilyl)oxy)-4-fluorophenyl)-1H-indazole

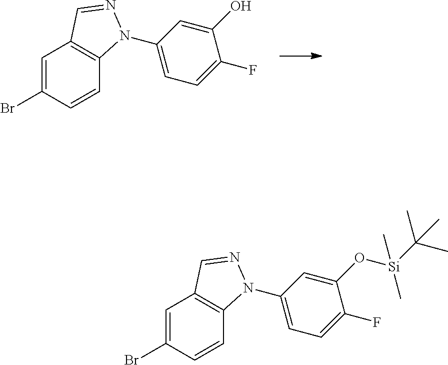 [SMILES: Oc1cc(-n2ncc3cc(Br)ccc32)ccc1F ; CC(C)(C)[Si](C)(C)Oc1cc(-n2ncc3cc(Br)ccc32)ccc1F]

TBSCl (486 mg, 3.22 mmol) and imidazole (292 mg, 4.29 mmol) were added to a solution of Intermediate 15 (330 mg, 1.07 mmol) in DMF (5 mL). The mixture was stirred at room temperature for 2 h, poured into H₂O (10 mL), and then extracted (3×10 mL EtOAc). The combined organic layers were washed (10 mL brine), dried (Na₂SO₄), filtered, and then concentrated. The residue was purified by silica gel chromatography (10-20% EtOAc/petroleum ether) to give 5-bromo-1-(3-((tert-butyldimethylsilyl)oxy)-4-fluorophenyl)-1H-indazole (410 mg, 90%) as a white solid. ¹H NMR (400 MHz, CDCl₃): δ 8.35 (s, 1H), 8.14 (d, 1H), 7.73 (d, 1H), 7.60-7.62 (m, 1H), 7.43-7.60 (m, 1H), 7.32-7.38 (m, 2H), 0.99 (s, 9H), 0.24 (s, 6H); LCMS: 421.0 [M+H]⁺.

### Intermediate 17

### 5-Bromo-1-(4-fluoro-3-(methoxymethoxy)phenyl)-1H-indazole

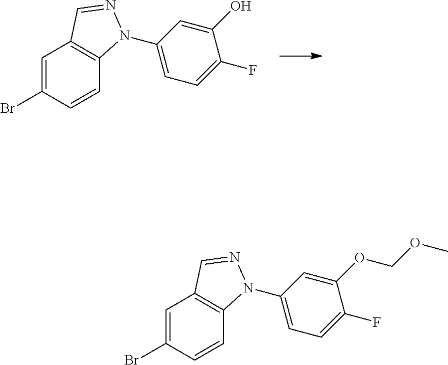 [SMILES: Oc1cc(-n2ncc3cc(Br)ccc32)ccc1F ; COCOc1cc(-n2ncc3cc(Br)ccc32)ccc1F]

Methoxymethyl chloride (0.52 mL, 6.70 mmol) was added dropwise to a solution of Intermediate 15 (1.7 g, 5.54 mmol) and DIPEA (1.46 mL, 8.30 mmol) in DCM (20 mL) at 0° C. The reaction mixture was stirred at room temperature for 2 h, poured into H₂O (100 mL), and then extracted (3×120 mL DCM). The combined organic layers were washed (100 mL brine), dried (Na₂SO₄), filtered, and then concentrated to give 5-bromo-1-(4-fluoro-3-(methoxymethoxy)phenyl)-1H-indazole (1.9 g) as a white solid. ¹H NMR (400 MHz, DMSO-d₆): δ 8.36 (d, 1H), 8.14 (d, 1H), 7.77 (d, 1H), 7.64-7.57 (m, 2H), 7.51-7.44 (m, 1H), 7.42-7.36 (m, 1H), 5.36 (s, 2H), 3.45 (s, 3H).

### Intermediate 18

### 5-Bromo-1-(3,4-difluoro-5-(methoxymethoxy)phenyl)-1H-indazole

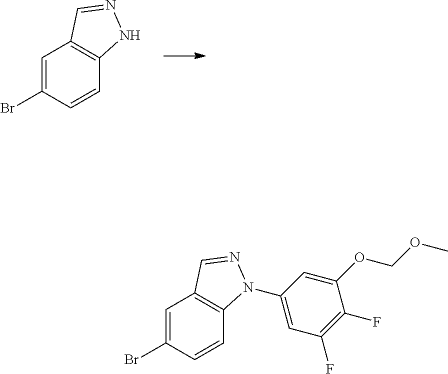 [SMILES: Brc1ccc2[nH]ncc2c1 ; COCOc1cc(-n2ncc3cc(Br)ccc32)cc(F)c1F]

Copper acetate (20.7 g, 114 mmol) was added to a mixture of 5-bromo-1H-indazole (15.0 g, 76.1 mmol), Intermediate 2 (22.9 g, 76.1 mmol), and diethylamine (78.4 mL, 761 mmol) in DCM (500 mL) at room temperature. The mixture was degassed with 3 vacuum/O₂ cycles, stirred at room temperature for 6 h under an O₂ atmosphere (balloon), poured into NH₃—H₂O (1000 mL), stirred for 0.5 h, and then extracted (3×1000 mL EtOAc). The combined organics were washed (1000 mL brine), dried (Na₂SO₄), filtered, and then concentrated. The residue was purified by silica gel chromatography (5% EtOAc/petroleum ether) to give 5-bromo-1-(3,4-difluoro-5-(methoxymethoxy)phenyl)-1H-indazole (8.0 g, 28%) as a yellow oil. ¹H NMR (400 MHz, DMSO-d₆): δ 8.39 (s, 1H), 8.15 (d, 1H), 7.85 (d, 1H), 7.63 (dd, 1H), 7.57-7.43 (m, 2H), 5.41 (s, 2H), 3.46 (s, 3H); LCMS: 369.1 [M+H]⁺.

The Intermediates below were synthesized in a similar manner to that described for Intermediate 18.

| Int | Structure | Name | [M + H]⁺ |
|---|---|---|---|
| 18.01 | 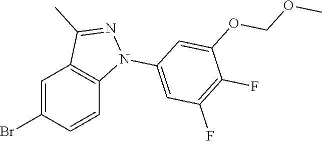 [SMILES: COCOc1cc(-n2nc(C)c3cc(Br)ccc32)cc(F)c1F] | 5-Bromo-1-(3,4-difluoro-5- (methoxymethoxy)phenyl)-3- methyl-1H-indazole | 366.8 [M − Me]⁻ |
| 18.02 | 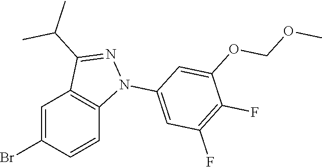 [SMILES: COCOc1cc(-n2nc(C(C)C)c3cc(Br)ccc32)cc(F)c1F] | 5-Bromo-1-(3,4-difluoro-5- (methoxymethoxy)phenyl)-3- isopropyl-1H-indazole | 411.1 |
| 18.03 | 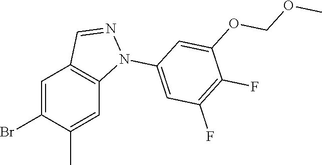 [SMILES: COCOc1cc(-n2ncc3cc(Br)c(C)cc32)cc(F)c1F] | 5-Bromo-1-(3,4-difluoro-5- (methoxymethoxy)phenyl)-6- methyl-1H-indazole | 383.1 |
| 18.04 | 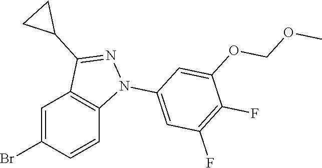 [SMILES: COCOc1cc(-n2nc(C3CC3)c3cc(Br)ccc32)cc(F)c1F] | 5-Bromo-3-cyclopropyl-1-(3,4- difluoro-5-(methoxymethoxy) phenyl)-1H-indazole | 409.0 |
| 18.05 | 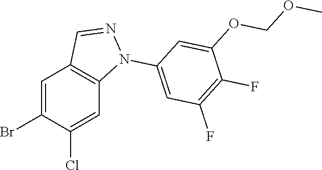 [SMILES: COCOc1cc(-n2ncc3cc(Br)c(Cl)cc32)cc(F)c1F] | 5-Bromo-6-chloro-1-(3,4- difluoro-5- (methoxymethoxy)phenyl)-1H- indazole | 403.0 |
| 18.06 | 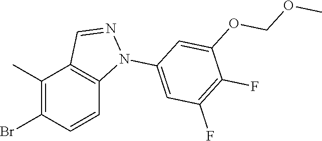 | 5-Bromo-1-(3,4-difluoro-5- (methoxymethoxy)phenyl)-4- methyl-1H-indazole | 383.1 |
| 18.07 | 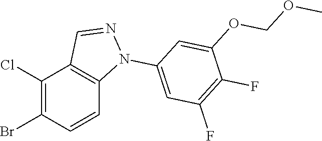 [SMILES: COCOc1cc(-n2ncc3c(Cl)c(Br)ccc32)cc(F)c1F] | 5-Bromo-4-chloro-1-(3,4- difluoro-5- (methoxymethoxy)phenyl)-1H- indazole | 403.0 |
| 18.08 | 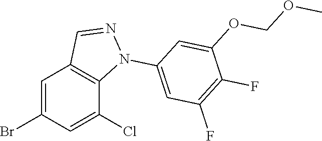 [SMILES: COCOc1cc(-n2ncc3cc(Br)cc(Cl)c32)cc(F)c1F] | 5-Bromo-7-chloro-1-(3,4- difluoro-5- (methoxymethoxy)phenyl)-1H- indazole | 403.1 |
| 18.09 | 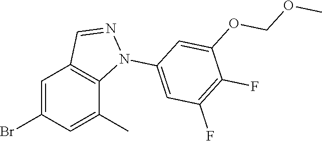 [SMILES: COCOc1cc(-n2ncc3cc(Br)cc(C)c32)cc(F)c1F] | 5-Bromo-1-(3,4-difluoro-5- (methoxymethoxy)phenyl)-7- methyl-1H-indazole | 383.1 |
| 18.10 | 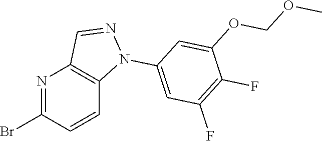 [SMILES: COCOc1cc(-n2ncc3nc(Br)ccc32)cc(F)c1F] | 5-Bromo-1-(3,4-difluoro-5- (methoxymethoxy)phenyl)-1H- pyrazolo[4,3-b]pyridine | 370.1 |
| 18.11 | 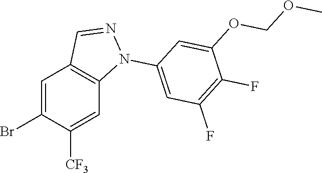 [SMILES: COCOc1cc(-n2ncc3cc(Br)c(C(F)(F)F)cc32)cc(F)c1F] | 5-Bromo-1-(3,4-difluoro-5- (methoxymethoxy)phenyl)-6- (trifluoromethyl)-1H-indazole | 437.0 |
| 18.12 | 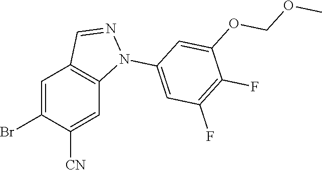 [SMILES: COCOc1cc(-n2ncc3cc(Br)c(C#N)cc32)cc(F)c1F] | 5-Bromo-1-(3,4-difluoro-5- (methoxymethoxy)phenyl)-1H- indazole-6-carbonitrile | 393.9 |
| 18.13 | 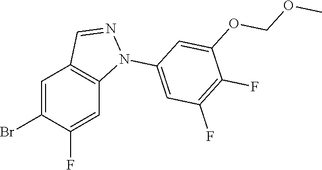 [SMILES: COCOc1cc(-n2ncc3cc(Br)c(F)cc32)cc(F)c1F] | 5-Bromo-1-(3,4-difluoro-5- (methoxymethoxy)phenyl)-6- fluoro-1H-indazole | 387.0 |
| 18.14 | 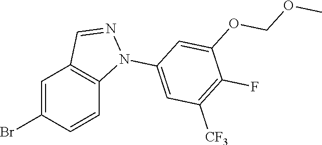 [SMILES: COCOc1cc(-n2ncc3cc(Br)ccc32)cc(C(F)(F)F)c1F] | 5-Bromo-1-(4-fluoro-3- (methoxymethoxy)-5- (trifluoromethyl)phenyl)-1H- indazole | 419.0 |
| 18.15 | 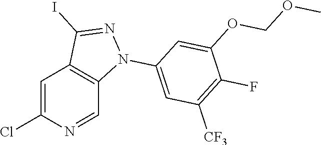 [SMILES: COCOc1cc(-n2nc(I)c3cc(Cl)ncc32)cc(C(F)(F)F)c1F] | 5-Chloro-1-(4-fluoro-3- (methoxymethoxy)-5- (trifluoromethyl)phenyl)-3-iodo- 1H-pyrazolo[3,4-c]pyridine | 502.0 |
| 18.16 | 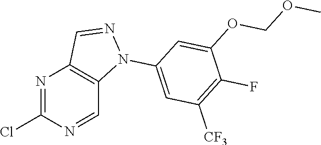 [SMILES: COCOc1cc(-n2ncc3nc(Cl)ncc32)cc(C(F)(F)F)c1F] | 5-Chloro-1-(4-fluoro-3- (methoxymethoxy)-5- (trifluoromethyl)phenyl)-1H- pyrazolo[4,3-d]pyrimidine | 377.0 |
| 18.17 | 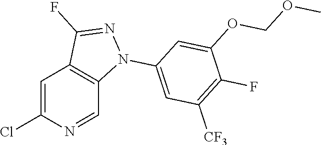 [SMILES: COCOc1cc(-n2nc(F)c3cc(Cl)ncc32)cc(C(F)(F)F)c1F] | 5-Chloro-3-fluoro-1-(4-fluoro-3- (methoxymethoxy)-5- (trifluoromethyl)phenyl)-1H- pyrazolo[3,4-c]pyridine | 394.0 |
| 18.18 | 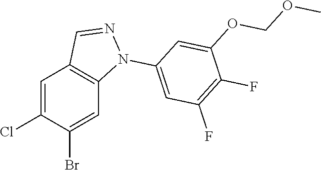 [SMILES: COCOc1cc(-n2ncc3cc(Cl)c(Br)cc32)cc(F)c1F] | 6-Bromo-5-chloro-1-(3,4- difluoro-5- (methoxymethoxy)phenyl)-1H- indazole | 403.0 |

### Intermediate 19

### 1-(3,4-Difluoro-5-(methoxymethoxy)phenyl)-5-(4,4,5,5-tetra methyl-1,3,2-dioxaborolan-2-yl)-1H-indazole

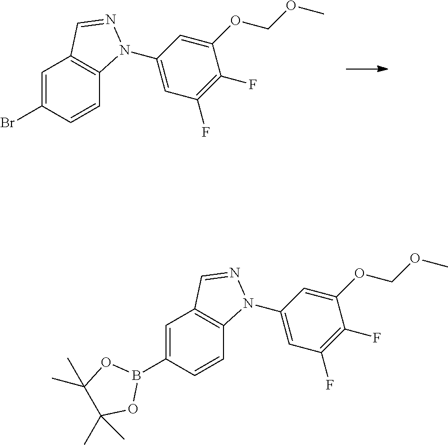

Pd(dppf)Cl₂ (496 mg, 0.68 mmol) was added to a mixture of Intermediate 18 (2.50 g, 6.77 mmol), bis(pinacolato)diboron (2.24 g, 8.80 mmol), KOAc (3.99 g, 40.6 mmol), and toluene (30 mL). The mixture was degassed and purged with N₂ three times, heated at 90° C. overnight, cooled to room temperature, poured into H₂O (50 mL), and then extracted (3×30 mL EtOAc). The combined organic layers were washed (50 mL brine), dried (Na₂SO₄), filtered, and then concentrated. The residue was purified by silica gel chromatography (2-10% EtOAc/petroleum ether) to give 1-(3,4-difluoro-5-(methoxymethoxy)phenyl)-5-(4,4,5,5-tetramethyl-1,3,2-dioxaborolan-2-yl)-1H-indazole (2.1 g, 74%) as a yellow solid. ¹H NMR (400 MHz, DMSO-d₆): δ 8.24 (s, 1H), 8.11 (s, 1H), 7.81-7.79 (m, 1H), 7.62-7.60 (m, 1H), 7.35-7.34 (m, 1H), 7.19-7.17 (m, 1H), 5.23 (s, 2H), 3.48 (s, 3H), 1.31 (s, 12H); LCMS: 417.1 [M+H]⁺.

The Intermediate below was synthesized from 5-bromo-1-(4-fluoro-5-hydroxyphenyl)-1H-indazole in a similar manner to that described for Intermediate 19.

| Int | Structure | Name | [M + H]⁺ |
|---|---|---|---|
| 19.01 | 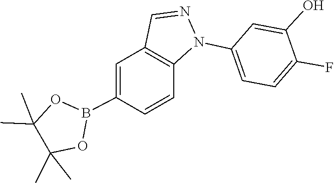 [SMILES: CC1(C)OB(c2ccc3c(cnn3-c3ccc(F)c(O)c3)c2)OC1(C)C] | 2-Fluoro-5-(5-(4,4,5,5-tetramethyl- 1,3,2-dioxaborolan-2-yl)-1H-indazol- 1-yl)phenol | 355.0 |

### Intermediate 20

### 1-(3,4-Difluoro-5-(methoxymethoxy)phenyl)-1H-indazol-5-ol

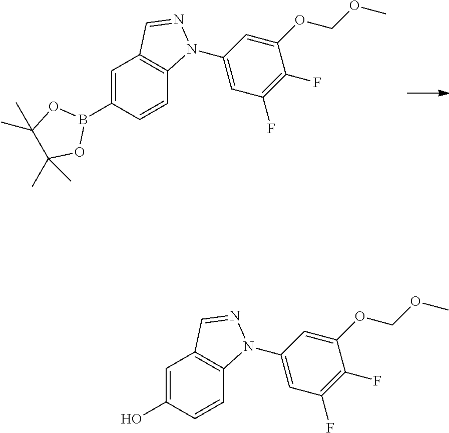

Hydrogen peroxide (21.8 g, 192 mmol, 30% purity) was added to a solution of Intermediate 19 (4.0 g, 9.61 mmol) and MeOH (80 mL). The mixture was stirred at room temperature for 4 h, quenched with saturated sodium sulfite solution (150 mL) dropwise, and then extracted (3×100 mL EtOAc). The combined organic layers were washed (100 mL brine), dried (Na₂SO₄), filtered, and then concentrated. The residue was purified by silica gel chromatography (25% EtOAc/petroleum ether) to give 1-(3,4-difluoro-5-(methoxymethoxy)phenyl)-1H-indazol-5-ol (2.5 g, 85%) as a white solid. ¹H NMR (400 MHz, DMSO-d₆): δ 9.48 (s, 1H), 8.20 (s, 1H), 7.74 (d, 1H), 7.53-7.41 (m, 2H), 7.11 (d, 1H), 7.05 (dd, 1H), 5.41 (s, 2H), 3.46 (s, 3H); LCMS: 307.1 [M+H]⁺.

### Intermediate 21

### 5-Bromo-1-(4-fluoro-3-methoxyphenyl)-1H-pyrazolo[3,4-c]pyridine

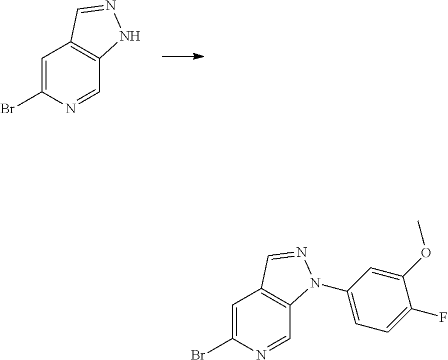 [SMILES: Brc1cc2cn[nH]c2cn1 ; COc1cc(-n2ncc3cc(Br)ncc32)ccc1F]

A mixture of 5-bromopyrazolo[3,4-c]pyridine (0.25 g, 1.26 mmol), 1-fluoro-4-iodo-2-methoxybenzene (0.38 g, 1.51 mmol), Cs₂CO₃ (1.03 g, 3.16 mmol), CuI (0.05 g, 0.25 mmol), trans-N,N-dimethylcyclohexane-1,2-diamine (0.16 mL, 1.01 mmol), and Tween20/water 2% (5.0 mL) were heated at 70° C. overnight. The reaction was diluted (EtOAc) and washed (brine). The organics were dried (MgSO₄) and concentrated. The residue was purified by silica gel chromatography (0-25% EtOAc/heptane) to give 5-bromo-1-(4-fluoro-3-methoxyphenyl)-1H-pyrazolo[3,4-c]pyridine (45 mg, 11%). LCMS 323.8 [M+H]⁺.

The Intermediates below were synthesized in a similar manner to that described for Intermediate 21.

| Int | Structure | Name | [M + H]⁺ |
|---|---|---|---|
| 21.01 (1) | 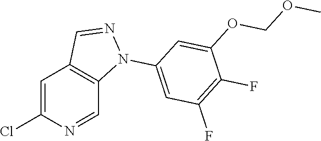 [SMILES: COCOc1cc(-n2ncc3cc(Cl)ncc32)cc(F)c1F] | 5-Chloro-1-(3,4-difluoro-5- (methoxymethoxy)phenyl)-1H- pyrazolo[3,4-c]pyridine | 326.0 |
| 21.02 (2) | 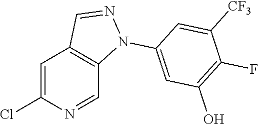 [SMILES: Oc1cc(-n2ncc3cc(Cl)ncc32)cc(C(F)(F)F)c1F] | 5-(5-Chloro-1H-pyrazolo[3,4- c]pyridine-1-yl)-2-fluoro-3- (trifluoromethyl)phenol | 331.8 |
| 21.03 (2,3) | 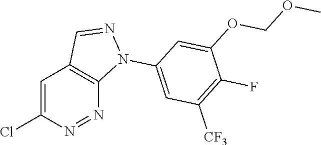 [SMILES: COCOc1cc(-n2ncc3cc(Cl)nnc32)cc(C(F)(F)F)c1F] | 5-Chloro-1-(4-fluoro-3- (methoxymethoxy)-5- (trifluoromethyl)phenyl)-1H- pyrazolo[3,4-c]pyridazine | 377.0 |
| 21.04 (4) | 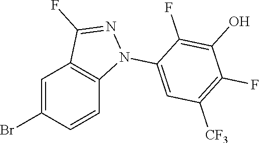 [SMILES: Oc1c(F)c(-n2nc(F)c3cc(Br)ccc32)cc(C(F)(F)F)c1F] | 3-(5-Bromo-3-fluoro-1H-indazol-1- yl)-2,6-difluoro-5- (trifluoromethyl)phenol | 411.3 |

Alternate conditions used: 1. Tween20/water was replaced with dioxane. 2. K₃PO₄, CuI, trans-N,N′-dimethylcyclohexane-1,2-diamine, bromide, toluene, 100° C. 3. KI added. 4. Tween20/water 2%, dioxane, 60° C., 2 h.

### Intermediate 22

### 5-(5-Chloro-1H-pyrazolo[4,3-d]pyrimidin-1-yl)-2-fluorophenol

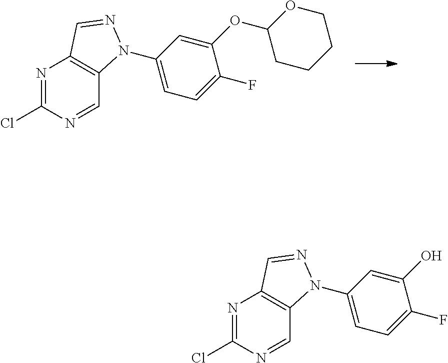 [SMILES: Fc1ccc(-n2ncc3nc(Cl)ncc32)cc1OC1CCCCO1 ; Oc1cc(-n2ncc3nc(Cl)ncc32)ccc1F]

A solution of Intermediate 14.07 (0.17 g, 0.49 mmol) in DCM (6.0 mL) and TFA (2.0 mL) was stirred at room temperature for 45 min and then concentrated. The residue was used crude in subsequent reactions. LCMS 264.8 [M+H]⁺.

### Intermediate 23

### 5-Chloro-6-cyclopropyl-1-(3,4-difluoro-5-(methoxymethoxy)phenyl)-1H-indazole

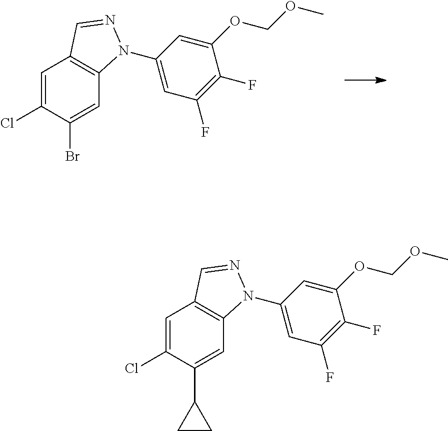 [SMILES: COCOc1cc(-n2ncc3cc(Cl)c(Br)cc32)cc(F)c1F ; COCOc1cc(-n2ncc3cc(Cl)c(C4CC4)cc32)cc(F)c1F]

Pd(dppf)Cl₂ (14 mg, 0.019 mmol) was added to a mixture of Intermediate 18.18 (150 mg, 0.37 mmol), cyclopropylboronic acid (160 mg, 1.86 mmol), Cs₂CO₃ (242 mg, 0.743 mmol), H₂O (1 mL), and dioxane (8 mL) at room temperature. The mixture was degassed with 3 vacuum/N₂ cycles, heated at 80° C. overnight, allowed to cool to room temperature, poured into H₂O (100 mL), and then extracted (3×100 mL EtOAc). The combined organic layers were washed (100 mL brine), dried (Na₂SO₄), filtered, and then concentrated. The residue was purified by prep-TLC (petroleum ether/EtOAc=3/1) to give 5-chloro-6-cyclopropyl-1-(3,4-difluoro-5-(methoxymethoxy)phenyl)-1H-indazole (90 mg, 59%) as a yellow oil. LCMS: 365.1 [M+H]⁺.

### Intermediate 24

### 5-Chloro-1-(4-fluoro-3-(methoxymethoxy)-5-(trifluoromethyl)phenyl)-3-methyl-1H-pyrazolo[3,4-c]pyridine

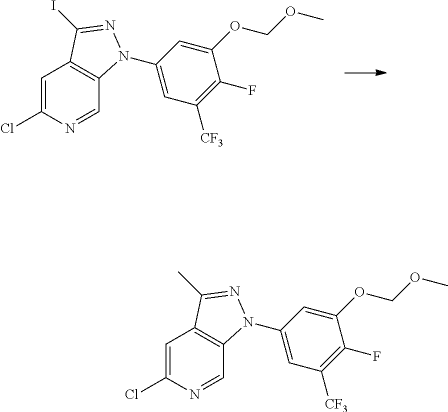 [SMILES: COCOc1cc(-n2nc(I)c3cc(Cl)ncc32)cc(C(F)(F)F)c1F ; COCOc1cc(-n2nc(C)c3cc(Cl)ncc32)cc(C(F)(F)F)c1F]

Pd(dppf)Cl₂ (55 mg, 0.07 mmol) was added to a mixture of Intermediate 18.15 (420 mg, 0.63 mmol), 2,4,6-trimethyl-1,3,5,2,4,6-trioxatriborinane (0.18 mL, 0.62 mmol, 50% pure in THF), and Cs₂CO₃ (1.08 g, 3.33 mmol) in dioxane (6 mL) and H₂O (0.6 mL)under N₂. The mixture was degassed with 3 vacuum/N₂ cycles, heated at 100° C. for 24 h, diluted (15 mL water), and then extracted (4×9 mL EtOAc). The combined organic layers were dried (Na₂SO₄), filtered, and then concentrated. The residue was purified by prep-TLC (petroleum ether/EtOAc=5:1) to give 5-chloro-1-(4-fluoro-3-(methoxymethoxy)-5-(trifluoromethyl)phenyl)-3-methyl-1H-pyrazolo[3,4-c]pyridine (136 mg, 56%) as a light yellow oil. ¹H NMR (400 MHz, DMSO-d₆): δ 9.10 (s, 1H), 8.10 (s, 1H), 7.99-7.97 (m, 1H), 7.72-7.70 (in, 1H), 5.49 (s, 2H), 3.48 (s, 3H), 2.62 (s, 3H); LCMS: 389.9 [M+H]⁺.

The Intermediates below were synthesized using the appropriate boronic acid in a similar manner to that described for Intermediate 24.

| Int | Structure | Name | [M + H]⁺ |
|---|---|---|---|
| 24.01 | 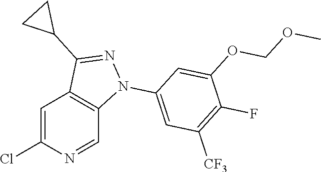 [SMILES: COCOc1cc(-n2nc(C3CC3)c3cc(Cl)ncc32)cc(C(F)(F)F)c1F] | 5-Chloro-3-cyclopropyl-1-(4-fluoro- 3-(methoxymethoxy)-5- (trifluoromethyl)phenyl)-1H- pyrazolo[3,4-c]pyridine | 416.1 |
| 24.02 (1,3) | 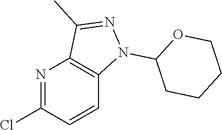 [SMILES: Cc1nn(C2CCCCO2)c2ccc(Cl)nc12] | 5-Chloro-3-methyl-1-(tetrahydro-2H- pyran-2-yl)-1H-pyrazolo[4,3- b]pyridine | 252.1 |
| 24.03 | 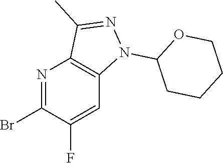 [SMILES: Cc1nn(C2CCCCO2)c2cc(F)c(Br)nc12] | 5-Bromo-6-fluoro-3-methyl-1- (tetrahydro-2H-pyran-2-yl)-1H- pyrazolo[4,3-b]pyridine | 314.0 |
| 24.05 (1,2,3) | 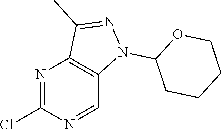 [SMILES: Cc1nn(C2CCCCO2)c2cnc(Cl)nc12] | 5-Chloro-3-methyl-1-(tetrahydro-2H- pyran-2-yl)-1H-pyrazolo[4,3- d]pyrimidine | 252.8 |
| 24.06 (1,2,3) | 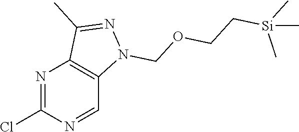 [SMILES: Cc1nn(COCC[Si](C)(C)C)c2cnc(Cl)nc12] | 5-Chloro-3-methyl-1-((2- (trimethylsilyl)ethoxy)methyl)-1H- pyrazolo[4,3-d]pyrimidine | 298.9 |
| 24.07 (1,3) | 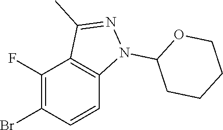 [SMILES: Cc1nn(C2CCCCO2)c2ccc(Br)c(F)c12] | 5-Bromo-4-fluoro-3-methyl-1- (tetrahydro-2H-pyran-2-yl)-1H- indazole | 313.0 |
| 24.08 (3,4) | 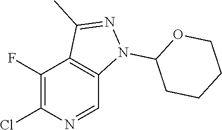 [SMILES: Cc1nn(C2CCCCO2)c2cnc(Cl)c(F)c12] | 5-Chloro-4-fluoro-3-methyl-1- (tetrahydro-2H-pyran-2-yl)-1H- pyrazolo[3,4-c]pyridine | 270.0 |
| 24.09 (1,2,3) | 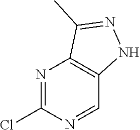 | 5-Chloro-3-methyl-1H-pyrazolo[4,3- d]pyrimidine | 168.8 |

Alternate conditions used: 1. K₃PO₄ instead of Cs₂CO₃. 2. Microwave, 120° C., 40 min-2 h. 3. Dioxane only as solvent. 4. Methylboronic acid used.

### Intermediate 24.10

### 3-(3-(Benzyloxy)-2,4-difluoro-5-(trifluoromethyl)phenyl)-6-bromo-3H-[1,2,3]triazolo[4,5-c]pyridine

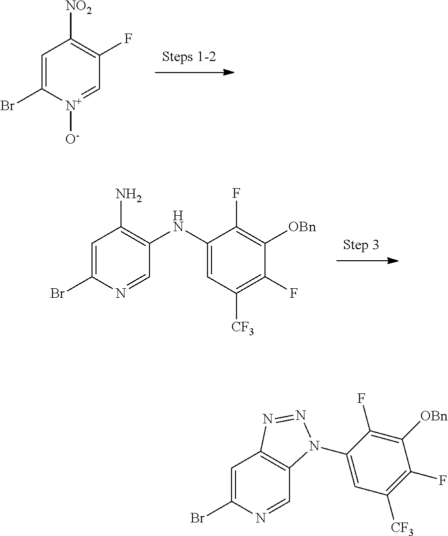 [SMILES: O=[N+]([O-])c1cc(Br)[n+]([O-])cc1F ; Nc1cc(Br)ncc1Nc1cc(C(F)(F)F)c(F)c(OCc2ccccc2)c1F ; Fc1c(-n2nnc3cc(Br)ncc32)cc(C(F)(F)F)c(F)c1OCc1ccccc1]

### Step 1: 5-((3-(Benzyloxy)-2,4-difluoro-5-(trifluoromethyl)phenyl)amino)-2-bromo-4-nitropyridine 1-oxide

A mixture of 2-bromo-5-fluoro-4-nitropyridine 1-oxide (2.02 g, 8.44 mmol), Intermediate 1.02 (1.71 g, 5.63 mmol), t-BuOK (1.89 g, 16.9 mmol), and DMSO (30 mL) was stirred at 75° C. for 12 h. The reaction mixture was cooled to rt, poured into H₂O (100 mL), and then extracted with EtOAc (3×100 mL). The organic layer was washed with brine (2×100 mL), dried (Na₂SO₄), filtered, concentrated, and then purified by silica gel chromatography (petroleum ether/ethyl acetate=30:1 to 3:1) to give 5-((3-(benzyloxy)-2,4-difluoro-5-(trifluoromethyl)phenyl)amino)-2-bromo-4-nitropyridine 1-oxide (630 mg, 21%) as a black/brown oil. ¹H NMR (400 MHz, DMSO-d₆): δ 9.35 (s, 1H), 8.58 (s, 1H), 8.11 (d, 1H), 7.68 (t, 1H), 7.48-7.36 (m, 5H), 5.28 (s, 2H); LCMS: 520.0 [M+H]⁺.

### Step 2: N³-(3-(Benzyloxy)-2,4-difluoro-5-(trifluoromethyl)phenyl)-6-bromopyridine-3,4-diamine

A mixture of 5-((3-(benzyloxy)-2,4-difluoro-5-(trifluoromethyl)phenyl)amino)-2-bromo-4-nitropyridine 1-oxide (0.63 g, 1.21 mmol), AcOH (10 mL), and H₂O (2.5 mL) was heated to 110° C. Iron powder (676 mg, 12.1 mmol) was added. The mixture was stirred at 110° C. for 2 h, allowed to cool to rt, poured into H₂O (20 mL), and then extracted with EtOAc (3×10 mL). The organic layer was washed with brine (10 mL), dried (Na₂SO₄), filtered, concentrated, and then purified by silica gel chromatography (petroleum ether/ethyl acetate=30:1 to 3:1) to give N³-(3-(benzyloxy)-2,4-difluoro-5-(trifluoromethyl)phenyl)-6-bromopyridine-3,4-diamine (412 mg, 710%) as a yellow oil. ¹H NMR (400 MHz, DMSO-d₆): δ 7.70 (s, 1H), 7.66 (s, 1H), 7.48-7.36 (m, 5H), 6.81 (s, 1H), 6.23 (t, 3H), 5.23 (s, 2H); LCMS: 473.9 [M+H]⁺.

### Step 3: 3-(3-(Benzyloxy)-2,4-difluoro-5-(trifluoromethyl)phenyl)-6-bromo-3H-[1,2,3]triazolo[4,5-c]pyridine

Sodium nitrite (86.2 mg, 1.25 mmol) in H₂O (0.4 mL) was added dropwise to a mixture of N³-(3-(benzyloxy)-2,4-difluoro-5-(trifluoromethyl)phenyl)-6-bromopyridine-3,4-diamine (395 mg, 0.833 mmol) in TFA (4 mL) at 0° C. The mixture was stirred at rt for 7 h, adjusted to pH=˜7 with sat. aq. NaHCO₃, and then extracted with DCM (3×20 mL). The organic layer was washed with brine (20 mL), dried (Na₂SO₄), filtered, concentrated, and then purified by silica gel chromatography (petroleum ether/ethyl acetate=50:1 to 5:1) to give 3-(3-(benzyloxy)-2,4-difluoro-5-(trifluoromethyl)phenyl)-6-bromo-3H-[1,2,3]triazolo[4,5-c]pyridine (240 mg, 59%) as a yellow oil. ¹H NMR (400 MHz, DMSO-d₆): δ 9.17 (s, 1H), 8.66 (s, 1H), 8.21 (t, 1H), 7.52-7.49 (m, 5H), 5.41 (s, 2H); LCMS: 484.9 [M+H]⁺.

The Intermediates below were synthesized from Intermediate 24.10 (Step 2) using the following conditions: triethoxymethane or 1,1,1-triethoxyethane, EtOH, HCl, 105° C., 3 h-overnight.

| Int | Structure | Name | [M + H]⁺ |
|---|---|---|---|
| 24.11 | 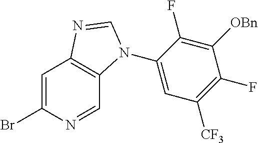 [SMILES: Fc1c(-n2cnc3cc(Br)ncc32)cc(C(F)(F)F)c(F)c1OCc1ccccc1] | 3-(3-(Benzyloxy)-2,4-difluoro-5- (trifluoromethyl)phenyl)-6-bromo- 3H-imidazo[4,5-c]pyridine | 484.0 |
| 24.12 | 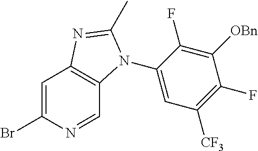 [SMILES: Cc1nc2cc(Br)ncc2n1-c1cc(C(F)(F)F)c(F)c(OCc2ccccc2)c1F] | 3-(3-(Benzyloxy)-2,4-difluoro-5- (trifluoromethyl)phenyl)-6-bromo-2- methyl-3H-imidazo[4,5-c]pyridine | 498.0 |

### Intermediate 25

### 5-(6-Bromobenzo[d]isoxazol-3-yl)-2-fluorophenol

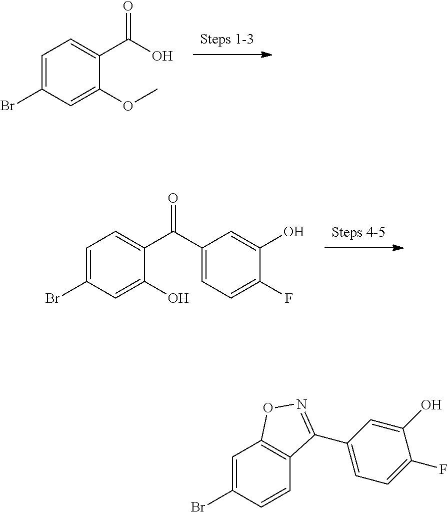 [SMILES: COc1cc(Br)ccc1C(=O)O ; O=C(c1ccc(F)c(O)c1)c1ccc(Br)cc1O ; Oc1cc(-c2noc3cc(Br)ccc23)ccc1F]

### Step 1: 4-Bromo-N,2-dimethoxy-N-methylbenzamide

HATU (3.62 g, 9.52 mmol) and DIPEA (4.5 mL, 26.0 mmol) were added to a solution of 4-bromo-2-methoxybenzoic acid (2.0 g, 8.66 mmol) and N, O-dimethylhydroxylamine hydrochloride (929 mg, 9.52 mmol) in DMF (20 mL). The mixture was stirred at room temperature overnight, poured into H₂O (30 mL), and then extracted (3×20 mL EtOAc). The combined organic layers were washed (20 mL brine), dried (Na₂SO₄), filtered, and then concentrated. The residue was purified by silica gel chromatography (2-20% EtOAc/petroleum ether) to give 4-bromo-N,2-dimethoxy-N-methylbenzamide (2.0 g, 84%) as a white solid. ¹H NMR (400 MHz, CDCl₃): δ 7.40 (s, 1H), 7.20-7.16 (m, 2H), 3.98 (s, 6H), 2.94 (s, 3H); LCMS: 273.9 [M+H]⁺.

### Step 2: (4-Bromo-2-methoxyphenyl)(4-fluoro-3-methoxyphenyl)methanone

n-Butyllithium (2.5 Min hexanes, 4.4 mL) was added to a solution of 4-bromo-1-fluoro-2-methoxybenzene (1.35 g, 6.57 mmol) in THF (10 mL) at −78° C. under N₂. After stirring the mixture for 1 h at −78° C., 4-bromo-N,2-dimethoxy-N-methylbenzamide (1.5 g, 5.47 mmol) in THF (10 mL) was added. The reaction mixture was stirred for 1 h, warmed to room temperature, stirred overnight, poured into saturated NH₄Cl (30 mL), and then extracted (3×35 mL EtOAc). The combined organic layers were washed (2×30 mL brine), dried (Na₂SO₄), filtered, and then concentrated. The residue was purified by silica gel chromatography (1-10% EtOAc/petroleum ether) to give (4-bromo-2-methoxyphenyl)(4-fluoro-3-methoxyphenyl)methanone (710 mg, 40%) as a yellow oil. ¹H NMR (400 MHz, CDCl₃): δ 7.65 (dd, 1H), 7.32 (s, 1H), 7.28-7.24 (m, 2H), 7.20 (s, 1H), 7.15-7.10 (m, 1H), 4.00 (s, 3H), 3.81 (s, 3H); LCMS: 338.9 [M+H]⁺.

### Step 3: (4-Bromo-2-hydroxyphenyl)(4-fluoro-3-hydroxyphenyl)methanone

Boron tribromide (1.0 mL, 10.5 mmol) was added to a mixture of (4-bromo-2-methoxyphenyl)(4-fluoro-3-methoxyphenyl)methanone (710 mg, 2.09 mmol) in DCM (10 mL) at −78° C. under N₂. The mixture was warmed to room temperature, stirred for 2 h, and then slowly poured into MeOH (20 mL). The pH was adjusted (pH=8) with saturated NaHCO₃ (˜20 mL) and the mixture was extracted (3×20 mL EtOAc). The combined organic layers were washed (20 mL brine), dried (Na₂SO₄), filtered, and then concentrated to give (4-bromo-2-hydroxyphenyl)(4-fluoro-3-hydroxyphenyl)methanone (510 mg) as a yellow solid. LCMS: 308.9 [M−H]⁻.

### Step 4: (z)-(4-Bromo-2-hydroxyphenyl)(4-fluoro-3-hydroxyphenyl)methanone oxime

A mixture of (4-bromo-2-hydroxyphenyl)(4-fluoro-3-hydroxyphenyl)methanone (740 mg, 2.38 mmol), hydroxylamine hydrochloride (496 mg, 7.14 mmol), and NaOAc (585 mg, 7.14 mmol) in EtOH (9 mL) and H₂O (3 mL) was heated at 95° C. overnight, cooled to room temperature, poured into H₂O (20 mL), and then extracted (3×10 mL EtOAc). The combined organic layers were washed (10 mL brine), dried (Na₂SO₄), filtered, and then concentrated to give (z)-(4-bromo-2-hydroxyphenyl)(4-fluoro-3-hydroxyphenyl)methanone oxime (700 mg) as a yellow solid. LCMS: 323.9 [M−H]⁻.

### Step 5: 5-(6-Bromobenzo[d]isoxazol-3-yl)-2-fluorophenol

A mixture of (z)-(4-bromo-2-hydroxyphenyl)(4-fluoro-3-hydroxyphenyl)methanone oxime (400 mg, 1.23 mmol), NaOAc (221 mg, 2.70 mmol), and Ac₂O (0.26 mL, 2.82 mmol) in DMF (8 mL) was heated at reflux for 3 h, cooled to room temperature, poured into H₂O (20 mL), and then extracted (3×10 mL EtOAc). The combined organic layers were washed (10 mL brine), dried (Na₂SO₄), filtered, and then concentrated. The residue was purified by silica gel chromatography (2-10% EtOAc/petroleum ether) to give 5-(6-bromobenzo[d]isoxazol-3-yl)-2-fluorophenol (150 mg, 40%) as a yellow solid. ¹H NMR (400 MHz, DMSO-d₆): δ 10.41 (s, 1H), 8.22 (d, 1H), 8.00 (d, 1H), 7.78-7.74 (m, 1H), 7.69-7.65 (m, 1H), 7.60-7.55 (m, 1H), 7.48-7.35 (m, 1H); LCMS: 305.9 [M−H]⁻.

### Intermediate 26

### 1-(4-Fluoro-3-methoxy phenyl)-5-(piperidin-4-yl)-1H-indazole hydrochloride

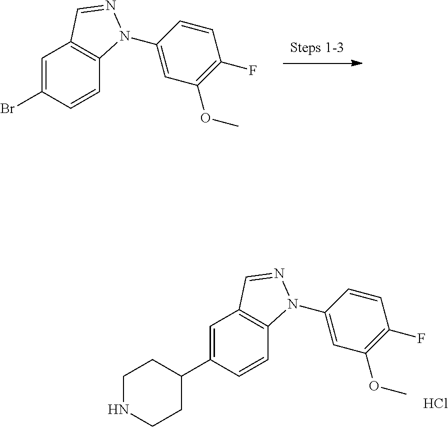 [SMILES: COc1cc(-n2ncc3cc(Br)ccc32)ccc1F ; COc1cc(-n2ncc3cc(C4CCNCC4)ccc32)ccc1F ; Cl]

### Step 1: tert-Butyl-4-(1-(4-fluoro-3-methoxyphenyl)-1H-indazol-5-yl)-5,6-dihydropyridine-1(2H)-carboxylate

Pd(PPh₃)₄ (360 mg, 0.311 mmol) was added to a mixture of Intermediate 14 (2.0 g, 6.23 mmol), tert-butyl-4-(4,4,5,5-tetramethyl-1,3,2-dioxaborolan-2-yl)-5,6-dihydropyridine-1(2H)-carboxylate (2.21 g, 7.16 mmol), Na₂CO₃ (2 M, 9.4 mL, 18.8 mmol), and dioxane (15 mL) under N₂ at room temperature. The mixture was degassed with 3 vacuum/N₂ cycles, stirred at 90° C. for 2.5 h, allowed to cool to room temperature, poured into H₂O (50 mL), and then extracted (3×50 mL EtOAc). The combined organic layers were washed (100 mL brine), dried (Na₂SO₄), filtered, and then concentrated. The residue was purified by silica gel chromatography (5% EtOAc/petroleum ether) to give tert-butyl-4-(1-(4-fluoro-3-methoxyphenyl)-1H-indazol-5-yl)-5,6-dihydropyridine-1(2H)-carboxylate (1.8 g, 68%) as a yellow oil. ¹H NMR (400 MHz, DMSO-d₆): δ 8.35 (s, 1H), 7.92-7.85 (m, 1H), 7.85-7.77 (m, 1H), 7.72-7.60 (m, 1H), 7.53-7.37 (m, 2H), 7.35-7.24 (m, 1H), 6.21 (s, 1H), 4.09-4.00 (m, 2H), 3.94 (s, 3H), 3.64-3.53 (m, 2H), 2.65-2.45 (m, 2H), 1.44 (s, 9H).

### Step 2: tert-Butyl-4-(1-(4-fluoro-3-methoxyphenyl)-1H-indazol-5-yl)piperidine-1-carboxylate

Palladium on carbon (1.8 g, 10%) was added to a solution of tert-butyl-4-(1-(4-fluoro-3-methoxyphenyl)-1H-indazol-5-yl)-5,6-dihydropyridine-1(2H)-carboxylate (1.8 g, 4.25 mmol) in MeOH (100 mL)under N₂ at room temperature. The suspension was degassed with 3 vacuum/H₂ cycles, stirred under H₂ (15 psi) for 2 h, and then filtered. The filtrate was concentrated to give tert-butyl-4-(1-(4-fluoro-3-methoxyphenyl)-1H-indazol-5-yl)piperidine-1-carboxylate (1.8 g) as a yellow oil. ¹H NMR (400 MHz, DMSO-d₆): δ 8.29 (s, 1H), 7.77 (d, 1H), 7.70 (s, 1H), 7.47 (dd, 1H), 7.44-7.36 (m, 2H), 7.34-7.24 (m, 1H), 4.20-4.05 (m, 2H), 3.93 (s, 3H), 2.92-2.76 (m, 3H), 1.86-1.75 (m, 2H), 1.65-1.50 (m, 2H), 1.42 (s, 9H); LCMS: 426.3 [M+H]⁺.

### Step 3: 1-(4-Fluoro-3-methoxyphenyl)-5-(piperidin-4-yl)-1H-indazole hydrochloride

Hydrochloric acid in MeOH (4 M, 50 mL) was added to tert-butyl-4-(1-(4-fluoro-3-methoxyphenyl)-1H-indazol-5-yl)piperidine-1-carboxylate (1.8 g, 4.23 mmol). The mixture was stirred at room temperature for 2 h then concentrated to give 1-(4-fluoro-3-methoxyphenyl)-5-(piperidin-4-yl)-1H-indazole hydrochloride (1.8 g) as a white solid. ¹H NMR (400 MHz, DMSO-d₆): δ 9.00-8.76 (m, 2H), 8.34 (s, 1H), 7.83 (d, 1H), 7.70 (s, 1H), 7.53-7.33 (m, 3H), 7.31-7.20 (m, 1H), 3.94 (s, 3H), 3.45-3.30 (m, 2H), 3.10-3.90 (m, 3H), 2.06-1.85 (m, 4H).

### Intermediate 27

### 2-Fluoro-5-(5-(piperazin-1-yl)-1H-pyrazolo[3,4-c]pyridazin 1-yl)-3-(trifluoromethyl)phenol

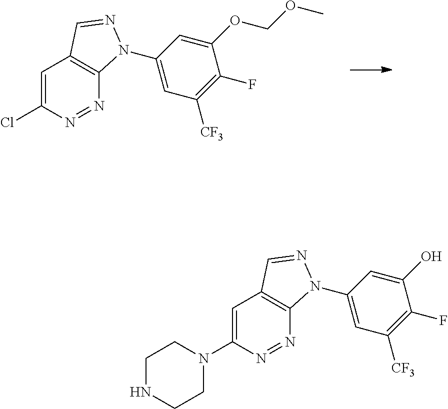 [SMILES: COCOc1cc(-n2ncc3cc(Cl)nnc32)cc(C(F)(F)F)c1F ; Oc1cc(-n2ncc3cc(N4CCNCC4)nnc32)cc(C(F)(F)F)c1F]

A mixture of Intermediate 21.03 (150 mg, 0.398 mmol) and piperazine (1.71 g, 19.9 mmol) in DMA (10 mL) was stirred at 160° C. for 8 h, allowed to cool to room temperature and then filtered. The filtrate was used directly without further purification for Compound 28.08. LCMS: 382.9 [M+H]⁺.

### Intermediate 28

### 5-(5-(Azetidin-3-yloxy)-1H-indazol-1-yl)-2,3-difluorophenol TFA Salt

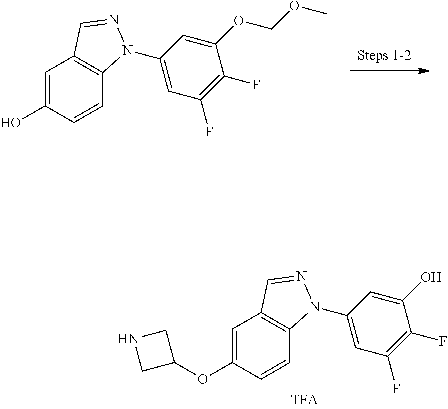 [SMILES: COCOc1cc(-n2ncc3cc(O)ccc32)cc(F)c1F ; Oc1cc(-n2ncc3cc(OC4CNC4)ccc32)cc(F)c1F]

### Step 1: tert-Butyl 3-((1-(3,4-difluoro-5-(methoxymethoxy)phenyl)-1H-indazol-5-yl)oxy)azetidine-1-carboxylate

Cs₂CO₃ (1.28 g, 3.92 mmol) was added to a solution of Intermediate 20 (400 mg, 1.31 mmol) and tert-butyl 3-iodoazetidine-1-carboxylate (444 mg, 1.57 mmol) in acetonitrile (10 mL). The mixture was heated at 80° C. for 3 h, cooled to room temperature, poured into water (30 mL), and then extracted (3×30 mL EtOAc). The combined organic layers were washed (2×30 mL brine), dried (Na₂SO₄), filtered, and then concentrated. The residue was purified by silica gel chromatography (1-10% EtOAc/petroleum ether) to give tert-butyl 3-((1-(3,4-difluoro-5-(methoxymethoxy)phenyl)-1H-indazol-5-yl)oxy)azetidine-1-carboxylate (490 mg, 81%) as a yellow oil. ¹H NMR (400 MHz, CDCl₃): δ 8.08 (s, 1H), 7.64 (d, 1H), 7.40-7.38 (m, 1H), 7.26-7.24 (m, 1H), 7.11 (d, 1H), 6.87 (s, 1H), 5.31 (s, 2H), 4.96-4.93 (m, 1H), 4.38-4.34 (m, 2H), 4.07-4.05 (m, 2H), 3.56 (s, 3H), 1.47 (s, 9H); LCMS: 462.2 [M+H]⁺.

### Step 2: 5-(5-(Azetidin-3-yloxy)-1H-indazol-1-yl)-2,3-difluorophenol TFA salt

Trifluoroacetic acid (2.0 mL) was added to a solution of tert-butyl 3-((1-(3,4-difluoro-5-(methoxymethoxy)phenyl)-1H-indazol-5-yl)oxy)azetidine-1-carboxylate (480 mg, 1.04 mmol) in DCM (10 mL). The mixture was stirred at rt for 2 h and then concentrated to give 5-(5-(azetidin-3-yloxy)-1H-indazol-1-yl)-2,3-difluorophenol TFA salt (590 mg). LCMS: 318.1 [M+H]⁺.

Note: In some instances, the free base was used: The TFA salt was basified with sat. aq. NaHCO₃. The aqueous layer was extracted with DCM, dried (Na₂SO₄), filtered, and then concentrated to give 5-(5-(azetidin-3-yloxy)-1H-indazol-1-yl)-2,3-difluorophenol as a free base.

The Intermediate below was synthesized from Intermediate 20 in a similar manner to that described for Intermediate 28.

| Int | Structure | Name | [M + H]⁺ |
|---|---|---|---|
| 28.01 | 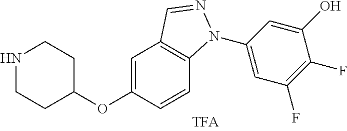 [SMILES: Oc1cc(-n2ncc3cc(OC4CCNCC4)ccc32)cc(F)c1F] | 2,3-Difluoro-5-(5-(piperidin-4- yloxy)-1H-indazol-1-yl)phenol TFA salt | 346.1 |

### Intermediate 29

### 1-(tert-Butyl) 4-methyl 4-(1-(3,4-difluoro-5-(methoxymethoxy)phenyl)-1H-indazol-5-yl)piperidine-1,4-dicarboxylate

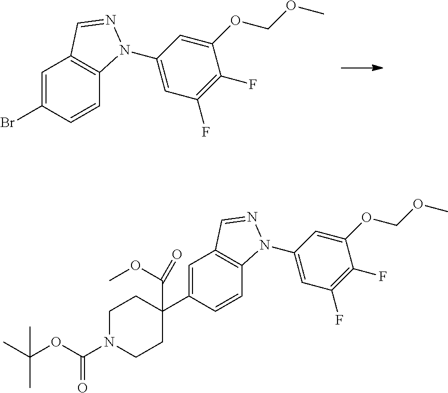

n-Butyllithium (2.5 M in hexanes, 9.8 mL) was added to a solution of dicyclohexylamine (4.9 mL, 24.7 mmol) in toluene (30 mL)under N₂ at −15° C. After stirring for 10 min, a solution of 1-tert-butyl-4-methyl piperidine-1,4-dicarboxylate (5.54 g, 22.8 mmol) in toluene (30 mL) was added. The mixture was stirred for 5 min. Intermediate 18 (7 g, 19 mmol), P^{t}Bu₃ (30 μL, 0.013 mmol, 10% purity), and Pd(OAc)₂ (1.2 mg, 0.0053 mmol) were added to the reaction. The mixture was heated at 100° C. for 1 h, allowed to cool to room temperature, poured into H₂O (100 mL), and then extracted (3×150 mL EtOAc). The combined organic layers were washed (150 mL brine), dried (Na₂SO₄), filtered, concentrated, and then purified by silica gel chromatography (20% EtOAc/petroleum ether) to give 1-(tert-butyl) 4-methyl 4-(1-(3,4-difluoro-5-(methoxymethoxy)phenyl)-1H-indazol-5-yl)piperidine-1,4-dicarboxylate (4.0 g, 39%) as a yellow oil. ¹H NMR (400 MHz, DMSO-d₆): 8.39 (d, 1H), 7.96-7.78 (m, 2H), 7.60-7.40 (m, 3H), 5.41 (s, 2H), 3.81 (d, 2H), 3.60 (s, 3H), 3.48 (s, 3H), 1.93-1.81 (m, 2H), 1.80-1.71 (m, 2H), 1.57-1.47 (m, 2H), 1.39 (s, 9H); LCMS: 532.3 [M+H]⁺.

### Intermediate 30

### tert-Butyl-4-cyano-4-(1-(3,4-difluoro-5-(methoxymethoxy)phenyl)-1H-indazol-5-yl)piperidine-1-carboxylate

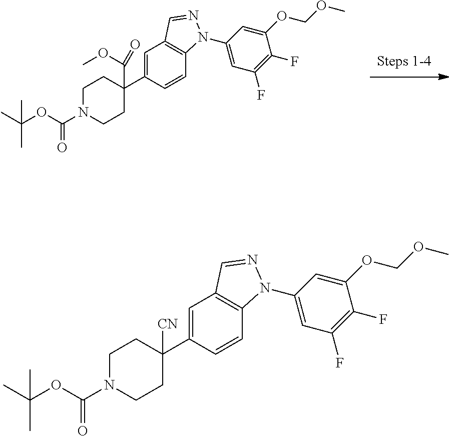

### Step 1: 1-(tert-Butoxycarbonyl)-4-(1-(3,4-difluoro-5-(methoxymethoxy)phenyl)-1H-indazol-5-yl)piperidine-4-carboxylic acid

A mixture of Intermediate 29 (500 mg, 0.94 mmol) and LiOH H₂O (395 mg, 9.41 mmol) in THF (10 mL), MeOH (5 mL) and H₂O (5 mL) was stirred at 50° C. for 4 h and cooled to room temperature. Aqueous hydrochloric acid (1 N) was added to the reaction mixture to adjust the pH-5. The mixture was poured into H₂O (30 mL) and extracted (3×50 mL EtOAc). The combined organic layers were washed (50 mL), dried (Na₂SO₄), filtered, and then concentrated to give 1-(tert-butoxycarbonyl)-4-(1-(3,4-difluoro-5-(methoxymethoxy)phenyl)-1H-indazol-5-yl)piperidine-4-carboxylic acid (450 mg) as a yellow oil. LCMS: 518.1 [M+H]⁺.

### Step 2: tert-Butyl-4-(chlorocarbonyl)-4-(1-(3,4-difluoro-5-(methoxymethoxy)phenyl)-1H-indazol-5-yl)piperidine-1-carboxylate

N-(Chloromethylene)-N-methylmethanaminium chloride (198 mg, 1.55 mmol) was added to a mixture of 1-(tert-butoxycarbonyl)-4-(1-(3,4-difluoro-5-(methoxymethoxy)phenyl)-1H-indazol-5-yl)piperidine-4-carboxylic acid (400 mg, 0.77 mmol) and K₂CO₃ (427 mg, 3.09 mmol) in dry toluene (10 mL)under N₂. The reaction mixture was stirred at room temperature for 1 h and filtered to give tert-butyl-4-(chlorocarbonyl)-4-(1-(3,4-difluoro-5-(methoxymethoxy)phenyl)-1H-indazol-5-yl)piperidine-1-carboxylate (10 mL in toluene).

### Step 3: tert-Butyl-4-carbamoyl-4-(1-(3,4-difluoro-5-(methoxymethoxy)phenyl)-1H-indazol-5-yl)piperidine-1-carboxylate

tert-Butyl-4-(chlorocarbonyl)-4-(1-(3,4-difluoro-5-(methoxymethoxy)phenyl)-1H-indazol-5-yl)piperidine-1-carboxylate (0.77 mmol) was added to a solution of NH₃·H₂O (3.0 mL, 23 mmol, 30% purity) and dry THE (10 mL) at 0° C. The mixture was warmed to room temperature, stirred for 0.5 h, poured into saturated NaHCO₃ (50 mL), and then extracted (3×60 mL EtOAc). The combined organic layers were washed (40 mL brine), dried (Na₂SO₄), filtered, and then concentrated. The residue was purified by silica gel chromatography (20% EtOAc/petroleum ether) to give tert-butyl-4-carbamoyl-4-(1-(3,4-difluoro-5-(methoxymethoxy)phenyl)-1H-indazol-5-yl)piperidine-1-carboxylate (300 mg, 75%) as a yellow oil. ¹H NMR (400 MHz, DMSO-d₆): δ 8.40 (s, 1H), 7.92-7.82 (m, 2H), 7.60-7.42 (m, 3H), 7.21 (d, 1H), 7.06 (d, 1H), 5.42 (s, 2H), 3.80-3.62 (m, 2H), 3.46 (s, 3H), 3.35-3.25 (m, 2H), 3.05-2.95 (m, 2H), 1.80-1.65 (m, 2H), 1.39 (s, 9H); LCMS: 517.2 [M+H]⁺.

### Step 4: tert-Butyl-4-cyano-4-(1-(3,4-difluoro-5-(methoxymethoxy)phenyl)-1H-indazol-5-yl)piperidine-1-carboxylate

Trifluoroacetic anhydride (708 mg, 3.37 mmol) was added to a mixture of tert-butyl-4-carbamoyl-4-(1-(3,4-difluoro-5-(methoxymethoxy)phenyl)-1H-indazol-5-yl)piperidine-1-carboxylate (290 mg, 0.561 mmol), Et₃N (620 μL, 4.49 mmol), and DCM (15 mL) at room temperature. The mixture was stirred for 1 h, poured into saturated NaHCO₃ (100 mL), and then extracted (3×100 mL EtOAc). The combined organic layers were washed (100 mL brine), dried (Na₂SO₄), filtered, and then concentrated. The residue was purified by silica gel chromatography (20% EtOAc/petroleum ether) to give tert-butyl-4-cyano-4-(1-(3,4-difluoro-5-(methoxymethoxy)phenyl)-1H-indazol-5-yl)piperidine-1-carboxylate (200 mg, 71%) as a yellow oil. ¹H NMR (400 MHz, DMSO-d₆): δ 8.45 (s, 1H), 8.07 (d, 1H), 7.98-7.90 (m, 1H), 7.75-7.68 (m, 1H), 7.62-7.50 (m, 2H), 5.42 (s, 2H), 3.46 (s, 3H), 4.30-4.10 (m, 2H), 3.10-2.90 (m, 2H), 2.15-2.05 (m, 2H), 2.10-1.90 (m, 2H), 1.43 (s, 9H); LCMS: 499.1 [M+H]⁺.

### Intermediate 31

### tert-Butyl 4-(1-(3,4-difluoro-5-(methoxymethoxy)phenyl)-1H-indazol-5-yl)-4-(hydroxymethyl)piperidine-1-carboxylate

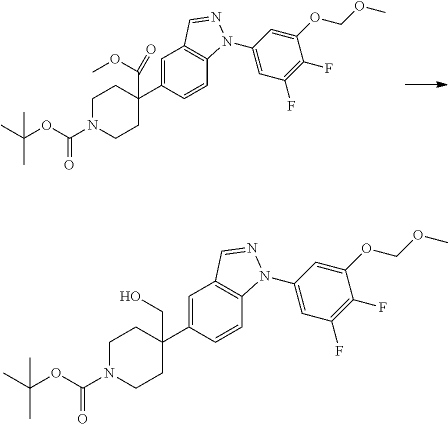

Lithium aluminum hydride (86 mg, 2.26 mmol) was slowly added to a solution of Intermediate 29 (480 mg, 0.90 mmol) in THF (10 mL) at 0° C. under N₂. The mixture was stirred at 0° C. for 1 h, quenched with H₂O (1 mL) and 15% NaOH (1 ml), and then filtered. The filtrate was concentrated and purified by prep-TLC (petroleum ether/EtOAc=1/2) to give tert-butyl 4-(1-(3,4-difluoro-5-(methoxymethoxy)phenyl)-1H-indazol-5-yl)-4-(hydroxymethyl)piperidine-1-carboxylate (213 mg, 22%) as a white solid. ¹H NMR (400 MHz, CDCl₃): δ 8.18 (s, 1H), 7.78-7.73 (m, 2H), 7.52-7.42 (m, 2H), 7.23-7.27 (m, 1H), 5.33 (s, 2H), 3.78-3.76 (m, 2H), 3.65 (d, 2H), 3.57 (s, 3H), 3.12-3.19 (m, 2H), 2.26-2.29 (m, 2H), 1.92-1.83 (m, 2H), 1.45 (s, 9H); LCMS: 504.2 [M+H]⁺.

### Intermediate 32

### tert-Butyl 4-(1-(3,4-difluoro-5-(methoxymethoxy)phenyl)-1H-indazol-5-yl)-4-(methoxymethyl)piperidine-1-carboxylate

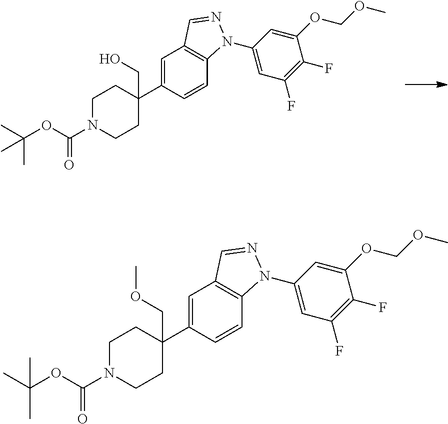

Sodium hydride (16 mg, 0.41 mmol, 60%) was carefully added to a mixture of Intermediate 31 (120 mg, 0.24 mmol) in THF (3.5 mL) at 0° C. and stirred for 10 min. Iodomethane (169 mg, 1.19 mmol) was added to the reaction and the mixture was stirred at 20° C. for 2 h. The reaction was quenched with saturated NH₄Cl (˜10 ml) and extracted (3×7 mL EtOAc). The combined organic layers were concentrated. The residue was purified by prep-TLC (petroleum ether/EtOAc=1.5/1) to give tert-butyl 4-[1-[3,4-difluoro-5-(methoxymethoxy)phenyl]indazol-5-yl]-4-(methoxymethyl)piperidine-1-carboxylate (115 mg, 88%) as a light yellow solid. ¹H NMR (400 MHz, CDCl₃): δ 8.16 (s, 1H), 7.76 (d, 1H), 7.73-7.70 (m, 1H), 7.52-7.50 (m, 1H), 7.44-7.42 (m, 1H), 7.27-7.25 (m, 1H), 5.32 (s, 2H), 3.75-3.73 (m, 2H), 3.57 (s, 3H), 3.40 (s, 2H), 3.23 (s, 3H), 3.13-3.09 (m, 2H), 2.24-2.20 (m, 2H), 1.98-1.90 (m, 2H), 1.45 (s, 9H); LCMS: 518.3 [M+H]⁺.

### Intermediate 33

### tert-Butyl 4-(1-(3,4-difluoro-5-(methoxymethoxy)phenyl)-1H-indazol-5-yl)-4-methylpiperidine-1-carboxylate

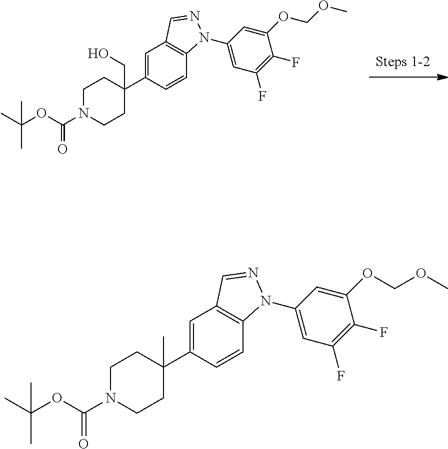

### Step 1: tert-Butyl 4-(1-(3,4-difluoro-5-(methoxymethoxy)phenyl)-1H-indazol-5-yl)-4-(((methylsulfonyl)oxy)methyl)piperidine-1-carboxylate

Triethylamine (402 mg, 3.97 mmol), DMAP (12 mg, 0.09 mmol), and then MsCl (273 mg, 2.38 mmol) were added to a solution of Intermediate 31 (1.0 g, 1.99 mmol) in DCM (20 mL) at 0° C. The mixture was stirred at room temperature for 2 h, poured into water (50 mL) and then extracted (3×50 mL EtOAc). The combined organic layers were washed (2×50 mL brine), dried (Na₂SO₄), filtered and concentrated. The residue was purified by silica gel chromatography (1-4% EtOAc/petroleum ether) to give tert-butyl 4-(1-(3,4-difluoro-5-(methoxymethoxy)phenyl)-1H-indazol-5-yl)-4-methylpiperidine-1-carboxylate (1.0 g, 86%) as a white solid. ¹H NMR (400 MHz, DMSO-d₆): δ 8.39 (s, 1H), 7.95 (s, 1H), 7.89 (d, 1H), 7.67 (s, 1H), 7.52 (d, 2H), 5.42 (s, 2H), 4.27 (s, 2H), 3.68 (d, 2H), 3.46 (s, 3H), 3.00 (s, 5H), 2.24 (d, 2H), 1.85 (s, 2H), 1.37 (s, 9H); LCMS: 582.2 [M+H]⁺.

### Step 2: tert-Butyl 4-(1-(3,4-difluoro-5-(methoxymethoxy)phenyl)-1H-indazol-5-yl)-4-methylpiperidine-1-carboxylate

LiEt₃BH (1 M in THF, 6.9 mL, 6.9 mmol) was added to a solution of tert-butyl 4-(1-(3,4-difluoro-5-(methoxymethoxy)phenyl)-1H-indazol-5-yl)-4-(((methylsulfonyl)oxy)methyl)piperidine-1-carboxylate (1.0 g, 1.72 mmol) in THF (20 mL) at room temperature. The mixture was stirred at 70° C. for 3 h, allowed to cool to room temperature, poured into water (50 mL), and then extracted (3×50 mL EtOAc). The combined organic layers were washed (2×50 mL brine), dried (Na₂SO₄), filtered, and then concentrated. The residue was purified by silica gel chromatography (1-5% EtOAc/petroleum ether) to give tert-butyl 4-(1-(3,4-difluoro-5-(methoxymethoxy)phenyl)-1H-indazol-5-yl)-4-methylpiperidine-1-carboxylate (200 mg) as a yellow oil. LCMS: 488.3 [M+H]⁺.

### Intermediate 34

### tert-Butyl 4-(1-(3-fluoro-4-(methoxymethoxy)pyridin-2-yl)-1H-indazol-5-yl)piperazine-1-carboxylate

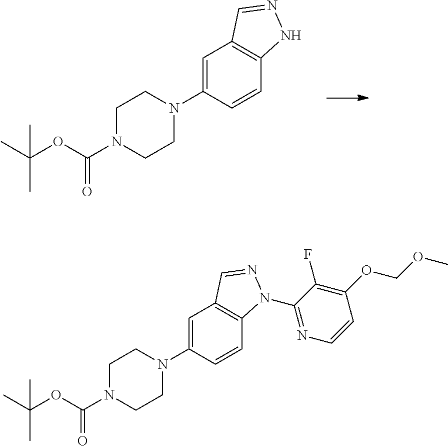 [SMILES: CC(C)(C)OC(=O)N1CCN(c2ccc3[nH]ncc3c2)CC1 ; COCOc1ccnc(-n2ncc3cc(N4CCN(C(=O)OC(C)(C)C)CC4)ccc32)c1F]

Sodium tert-butoxide (293 mg, 3.05 mmol) and BrettPhos Pd G4 (94 mg, 102 μmol) were added to a mixture of Intermediate 3 (240 mg, 1.02 mmol) and Intermediate 13.06 (307 mg, 1.02 mmol) in toluene (5 mL). The mixture was stirred at 100° C. overnight under N₂, allowed to cool to room temperature, slowly poured into H₂O (30 mL), and then extracted (3×30 mL EtOAc). The combined organic layers were washed (70 mL brine), dried (Na₂SO₄), filtered, concentrated, and then purified by silica gel chromatography (50% EtOAc/petroleum ether) to give tert-butyl 4-(1-(3-fluoro-4-(methoxymethoxy)pyridine-2-yl)-1H-indazol-5-yl)piperazine-1-carboxylate (81 mg, 17%) as a yellow oil.

The Intermediates below were synthesized from Intermediate 13.06 in a similar manner to that described for Intermediate 34.

| Int | Structure | Name | [M + H]⁺ |
|---|---|---|---|
| 34.01 | 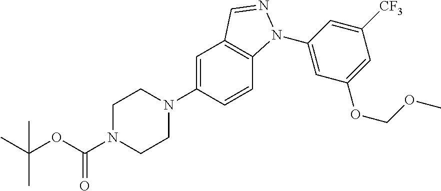 [SMILES: COCOc1cc(-n2ncc3cc(N4CCN(C(=O)OC(C)(C)C)CC4)ccc32)cc(C(F)(F)F)c1] | tert-Butyl 4-(1-(3- (methoxymethoxy)-5- (trifluoromethyl)phenyl)- 1H-indazol- 5-yl)piperazine-1-carboxylate | 507.3 |
| 34.02 | 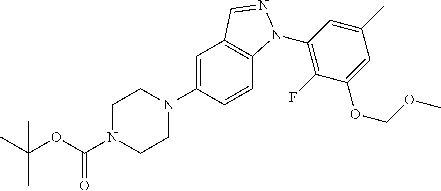 [SMILES: COCOc1cc(C)cc(-n2ncc3cc(N4CCN(C(=O)OC(C)(C)C)CC4)ccc32)c1F] | tert-Butyl 4-(1-(2-fluoro-3- (methoxymethoxy)- 5-methylphenyl)- 1H-indazol-5-yl)piperazine-1- carboxylate | 471.3 |

### Compound 1

### 2-Chloro-4-(1-(2-fluoro-5-hydroxyphenyl)-1H-indazol-5-yl)phenol

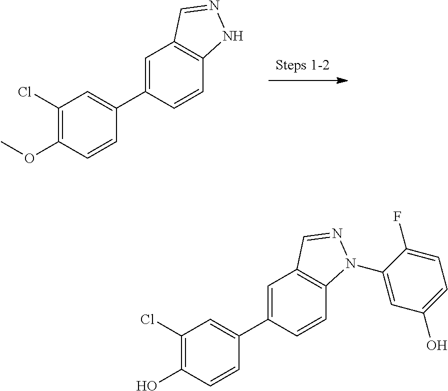 [SMILES: COc1ccc(-c2ccc3[nH]ncc3c2)cc1Cl ; Oc1ccc(F)c(-n2ncc3cc(-c4ccc(O)c(Cl)c4)ccc32)c1]

### Step 1: 5-(3-Chloro-4-methoxyphenyl)-1-(2-fluoro-5-methoxyphenyl)-1H-indazole

A mixture of Intermediate 10 (250 mg, 0.97 mmol), 4-fluoro-3-iodo-1-methoxybenzene (319 mg, 1.27 mmol), N1,N2-dimethylethane-1,2-diamine (32 mg, 0.36 mmol), CuI (21 mg, 0.11 mmol), K₃PO₄ (413 mg, 1.95 mmol), and DMF (3 mL) was degassed by bubbling N₂ through the suspension for 10 min, heated at 85° C. for 2 days, allowed to cool to room temperature, diluted (20 mL EtOAc), washed (20 mL water and then 20 mL brine), dried (Na₂SO₄), and then concentrated. The residue was purified by silica gel chromatography (0-15% EtOAc/hexanes) to give 5-(3-chloro-4-methoxyphenyl)-1-(2-fluoro-5-methoxyphenyl)-1H-indazole (70 mg, 19%) as a white solid. LCMS: 383.0 [M+H]⁺.

### Step 2: 2-Chloro-4-(1-(2-fluoro-5-hydroxyphenyl)-1H-indazol-5-yl)phenol

A mixture of 5-(3-chloro-4-methoxyphenyl)-1-(2-fluoro-5-methoxyphenyl)-1H-indazole (70 mg, 0.18 mmol) and DCM (3 mL) was cooled in a dry ice/acetone bath. Boron tribromide (1 M in DCM, 1.0 mL, 1.0 mmol) was added. The reaction was warmed to 0° C., stirred at 0° C. overnight, cooled in a dry ice/acetone bath, quenched with methanol (2 mL), allowed to warm to room temperature, and then concentrated. The residue was purified by prep-HPLC to give 2-chloro-4-(1-(2-fluoro-5-hydroxyphenyl)-1H-indazol-5-yl)phenol (46 mg, 71%) as a white solid. ¹H NMR (400 MHz, DMSO-d₆): δ 10.30 (s, 1H), 9.89 (s, 1H), 8.41 (d, J=0.9 Hz, 1H), 8.08 (d, J=1.0 Hz, 1H), 7.74 (dd, J=1.8, 8.9 Hz, 1H), 7.70 (d, J=2.3 Hz, 1H), 7.52 (dd, J=2.3, 8.5 Hz, 1H), 7.45 (dd, J=3.4, 8.8 Hz, 1H), 7.36 (dd, J=9.0, 10.4 Hz, 1H), 7.08 (d, J=8.4 Hz, 1H), 7.01 (dd, J=2.9, 6.2 Hz, 1H), 6.91 (td, J=3.5, 8.9 Hz, 1H); LCMS 354.9 [M+H]⁺.

The Compounds below were synthesized in a similar manner as described for Compound 1.

| Cmpd | Structure | Name | [M + H]⁺ |
|---|---|---|---|
| 1.01 | 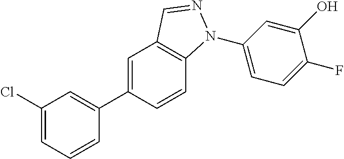 [SMILES: Oc1cc(-n2ncc3cc(-c4cccc(Cl)c4)ccc32)ccc1F] | 5-(5-(3-Chlorophenyl)-1H-indazol-1- yl)-2-fluorophenol | 339.0 |
| 1.02 (1,2) | 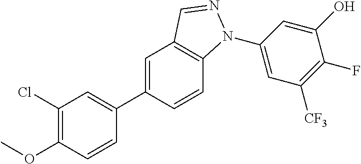 [SMILES: COc1ccc(-c2ccc3c(cnn3-c3cc(O)c(F)c(C(F)(F)F)c3)c2)cc1Cl] | 5-(5-(3-Chloro-4-methoxyphenyl)- 1H-indazol-1-yl)-2-fluoro-3- (trifluoromethyl)phenol | 437.0 |
| 1.03 (1,2) | 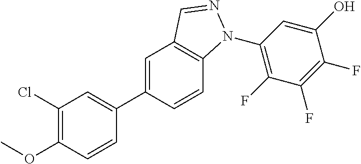 [SMILES: COc1ccc(-c2ccc3c(cnn3-c3cc(O)c(F)c(F)c3F)c2)cc1Cl] | 5-(5-(3-Chloro-4-methoxyphenyl)- 1H-indazol-1-yl)-2,3,4- trifluorophenol | 405.1 |
| 1.04 (1) | 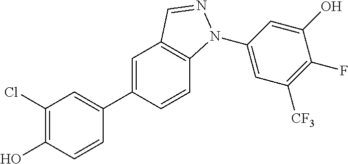 [SMILES: Oc1ccc(-c2ccc3c(cnn3-c3cc(O)c(F)c(C(F)(F)F)c3)c2)cc1Cl] | 5-(5-(3-Chloro-4-hydroxyphenyl)- 1H-indazol-1-yl)-2-fluoro-3- (trifluoromethyl)phenol | 423.0 |
| 1.05 (1) | 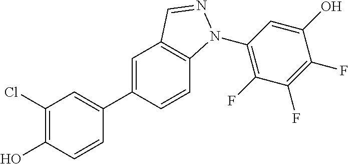 [SMILES: Oc1ccc(-c2ccc3c(cnn3-c3cc(O)c(F)c(F)c3F)c2)cc1Cl] | 5-(5-(3-Chloro-4-hydroxyphenyl)- 1H-indazol-1-yl)-2,3,4- trifluorophenol | 390.9 |
| 1.06 | 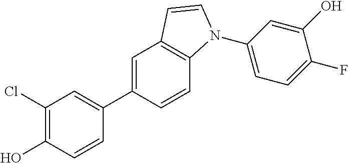 [SMILES: Oc1cc(-n2ccc3cc(-c4ccc(O)c(Cl)c4)ccc32)ccc1F] | 2-Chloro-4-(1-(4-fluoro-3- hydroxyphenyl)-1H-indol-5- yl)phenol | 354.0 |

Alternate conditions used: 1. Step 1: trans-N, N′-dimethylcyclohexane-1,2-diamine, K₃PO₄, CuI, toluene, ArBr; 2. Step 2 was omitted.

### Compound 2

### 5-(5-(3-Chloro-4-methoxyphenyl)-1H-indazol-1-yl)-2-fluorophenol

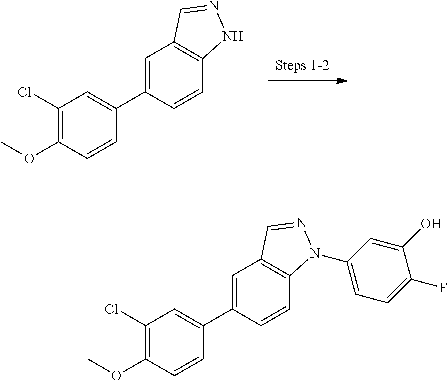 [SMILES: COc1ccc(-c2ccc3[nH]ncc3c2)cc1Cl ; COc1ccc(-c2ccc3c(cnn3-c3ccc(F)c(O)c3)c2)cc1Cl]

### Step 1: 1-(3-(Benzyloxy)-4-fluorophenyl)-5-(3-chloro-4-methoxyphenyl)-1H-indazole

Copper acetate (408 mg, 2.25 mmol) was added to a mixture of Intermediate 10 (213 mg, 0.83 mmol), 3-benzyloxy-4-fluorophenylboronic acid (417 mg, 1.69 mmol), pyridine (0.2 mL, 2.47 mmol), and DCM (10 mL) at room temperature. The mixture was stirred overnight and then filtered through a Celite plug. The filter cake was washed (10 mL DCM), and the filtrate was concentrated. The residue was purified by silica gel chromatography (0-15% EtOAc/hexanes) to give 1-(3-(benzyloxy)-4-fluorophenyl)-5-(3-chloro-4-methoxyphenyl)-1H-indazole (171 mg, 33%) as a white foam. ¹H NMR (400 MHz, DMSO-d₆): δ 8.41-8.39 (m, 1H), 8.15 (d, J=1.1 Hz, 1H), 7.81 (d, J=2.3 Hz, 1H), 7.78-7.74 (m, 1H), 7.73-7.65 (m, 2H), 7.62-7.57 (m, 1H), 7.54-7.49 (m, 2H), 7.49-7.44 (m, 3H), 7.42-7.37 (m, 1H), 7.37-7.30 (m, 1H), 7.29-7.26 (m, 1H), 5.35 (s, 2H), 3.92 (s, 3H); LCMS 459.5 [M+H]⁺.

### Step 2: 5-(5-(3-Chloro-4-methoxyphenyl)-1H-indazol-1-yl)-2-fluorophenol

Palladium on carbon (10%, 20 mg) in THF (2 mL) was added to a mixture of 1-(3-(benzyloxy)-4-fluorophenyl)-5-(3-chloro-4-methoxyphenyl)-1H-indazole (165 mg, 0.36 mmol) and THE (3 mL) at room temperature. The mixture was stirred under a balloon of hydrogen for 2 h and then filtered through a Celite plug. The filter cake was rinsed with 5 mL THF. The filtrate was concentrated. The residue was triturated in methanol (3 mL) to give 5-(5-(3-chloro-4-methoxyphenyl)-1H-indazol-1-yl)-2-fluorophenol (90 mg, 68%) as a white solid. ¹H NMR (400 MHz, DMSO-d₆): δ 10.40 (br s, 1H), 8.38 (s, 1H), 8.15 (s, 1H), 7.86-7.79 (m, 3H), 7.70 (dd, J=2.3, 8.6 Hz, 1H), 7.40-7.34 (m, 2H), 7.27 (d, J=8.7 Hz, 1H), 7.21 (dd, J=4.4, 7.2 Hz, 1H), 3.92 (s, 3H); LCMS 368.9 [M+H]⁺.

The Compound below was synthesized in a similar manner as described for Compound 2.

| Cmpd | Structure | Name | [M + H]⁺ |
|---|---|---|---|
| 2.01 (1) | 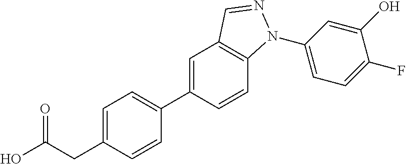 [SMILES: O=C(O)Cc1ccc(-c2ccc3c(cnn3-c3ccc(F)c(O)c3)c2)cc1] | 2-(4-(1-(4-Fluoro-3-hydroxyphenyl)- 1H-indazol-5-yl)phenyl)acetic acid | 363.0 |

Alternate conditions used: 1. From carboxylic ester hydrolysis (NaOH:methanol:THF).

### Compound 3

### 2-Chloro-4-(1-(3-fluoro-5-hydroxyphenyl)-1H-indazol-5-yl)phenol

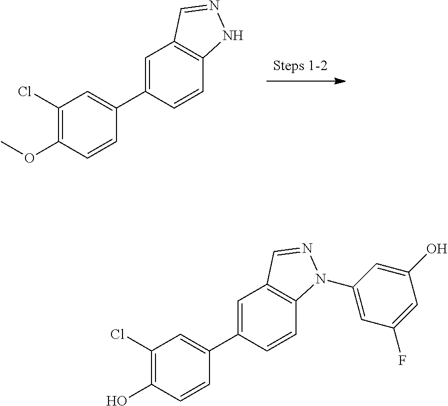 [SMILES: COc1ccc(-c2ccc3[nH]ncc3c2)cc1Cl ; Oc1cc(F)cc(-n2ncc3cc(-c4ccc(O)c(Cl)c4)ccc32)c1]

### Step 1: 5-(2-Chloro-4-methoxyphenyl)-1-(4-fluoro-3-methoxyphenyl)-1H-indazole

Copper acetate (357 mg, 1.97 mmol) was added to a mixture of Intermediate 10 (250 mg, 0.97 mmol), 3-methoxy-5-fluorophenylboronic acid (255 mg, 1.50 mmol), pyridine (0.2 mL, 2.47 mmol), and DCM (10 mL) at room temperature. The mixture was stirred for 2 days and then filtered through a Celite plug. The filter cake was washed (10 mL DCM), and the filtrate was concentrated. The residue was purified by silica gel chromatography (0-15% EtOAc/hexanes) to give 5-(2-chloro-4-methoxyphenyl)-1-(4-fluoro-3-methoxyphenyl)-1H-indazole (173 mg, 33%) as a white solid. ¹H NMR (400 MHz, DMSO-d₆): δ 8.46-8.44 (m, 1H), 8.17 (d, J=1.1 Hz, 1H), 7.99 (d, J=8.8 Hz, 1H), 7.85-7.80 (m, 2H), 7.71 (dd, J=2.3, 8.6 Hz, 1H), 7.31-7.23 (m, 2H), 7.22 (s, 1H), 6.92 (td, J=2.3, 10.9 Hz, 1H), 3.94-3.91 (m, 3H), 3.90-3.87 (m, 3H); LCMS 382.9 [M+H]⁺.

### Step 2: 2-Chloro-4-(1-(3-fluoro-5-hydroxyphenyl)-1H-indazol-5-yl)phenol

A mixture of 5-(2-chloro-4-methoxyphenyl)-1-(4-fluoro-3-methoxyphenyl)-1H-indazole (100 mg, 0.26 mmol) and DCM (3 mL) was cooled in a dry ice/acetone bath. Boron tribromide (1 M in DCM, 1.5 mL, 1.5 mmol) was added. The reaction was stirred at 0° C. overnight, cooled in a dry ice/acetone bath, quenched with methanol (3 mL), allowed to warm to room temperature, and then concentrated. The residue was purified by prep-HPLC to give 2-chloro-4-(1-(3-fluoro-5-hydroxyphenyl)-1H-indazol-5-yl)phenol (30 mg, 33.) as a white solid. ¹H NMR (400 MHz, DMSO-d₆): δ 10.39 (br s, 1H), 10.32 (s, 1H), 8.42-8.40 (m, 1H), 8.11 (d, J=1.1 Hz, 1H), 7.92 (d, J=8.9 Hz, 1H), 7.80 (dd, J=1.7, 8.9 Hz, 1H), 7.72 (d, J=2.3 Hz, 1H), 7.54 (d d, J=2.3, 8.6 Hz, 1H), 7.12-7.07 (m, 3H), 6.61 (td, J˜ 2.2, 10.7 Hz, 1H); LCMS 354.9 [M+H]⁺.

The Compounds below were synthesized in a similar manner to that described for Compound 3.

| Cmpd | Structure | Name | [M + H]⁺ |
|---|---|---|---|
| 3.01 | 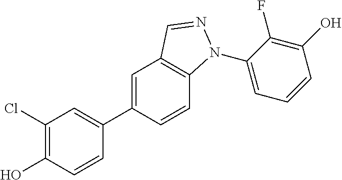 [SMILES: Oc1ccc(-c2ccc3c(cnn3-c3cccc(O)c3F)c2)cc1Cl] | 3-(5-(3-Chloro-4-hydroxyphenyl)- 1H-indazol-1-yl)-2-fluorophenol | 354.9 |
| 3.02 | 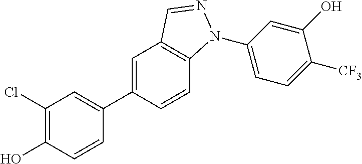 [SMILES: Oc1ccc(-c2ccc3c(cnn3-c3ccc(C(F)(F)F)c(O)c3)c2)cc1Cl] | 2-Chloro-4-(1-(3-hydroxy-4- (trifluoromethyl)phenyl)-1H-indazol- 5-yl)phenol | 405.0 |
| 3.03 | 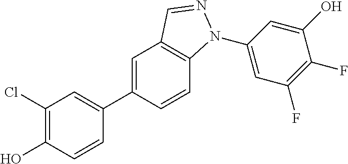 [SMILES: Oc1ccc(-c2ccc3c(cnn3-c3cc(O)c(F)c(F)c3)c2)cc1Cl] | 5-(5-(3-Chloro-4-hydroxyphenyl)- 1H-indazol-1-yl)-2,3-difluorophenol | 373.0 |
| 3.04 | 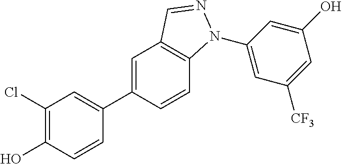 [SMILES: Oc1cc(-n2ncc3cc(-c4ccc(O)c(Cl)c4)ccc32)cc(C(F)(F)F)c1] | 2-Chloro-4-(1-(3-hydroxy-5- (trifluoromethyl)phenyl)-1H-indazol- 5-yl)phenol | 405.0 |
| 3.05 | 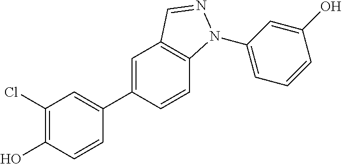 [SMILES: Oc1cccc(-n2ncc3cc(-c4ccc(O)c(Cl)c4)ccc32)c1] | 2-Chloro-4-(1-(3-hydroxyphenyl)- 1H-indazol-5-yl)phenol | 336.9 |

### Compound 4

### 2-Chloro-4-(1-(4-fluoro-3-hydroxyphenyl)-1H-indazol-5-yl)phenol

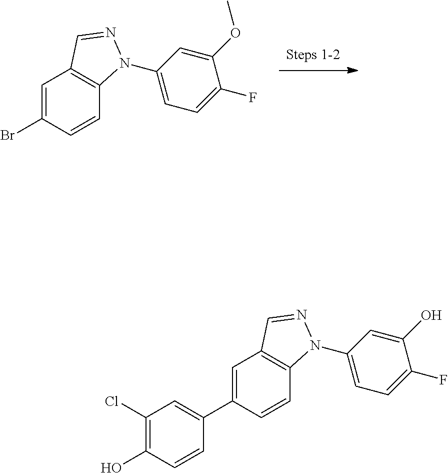 [SMILES: COc1cc(-n2ncc3cc(Br)ccc32)ccc1F ; Oc1cc(-n2ncc3cc(-c4ccc(O)c(Cl)c4)ccc32)ccc1F]

### Step 1: 5-(3-Chloro-4-methoxyphenyl)-1-(4-fluoro-3-methoxyphenyl)-1H-indazole

A mixture of Intermediate 14 (130 mg, 0.40 mmol), 4-fluoro-3-methoxyphenylboronic acid (115 mg, 0.62 mmol), Pd(PPh₃)₄ (50 mg, 0.04 mmol), Na₂CO₃ (2 M, 0.4 mL, 0.8 mmol), and dioxane (1 mL) was heated at 90° C. for 40 min, allowed to cool to room temperature, diluted (20 mL EtOAc), and then washed (20 mL water and then 20 mL brine). The organic layer was dried (Na₂SO₄), concentrated, and then purified by silica gel chromatography (0-15% EtOAc/hexanes) to give 5-(3-chloro-4-methoxyphenyl)-1-(4-fluoro-3-methoxyphenyl)-1H-indazole (150 mg, 99%) as a white solid. LCMS 383.0 [M+H]⁺.

### Step 2: 2-Chloro-4-(1-(4-fluoro-3-hydroxyphenyl)-1H-indazol-5-yl)phenol

A mixture of 5-(3-chloro-4-methoxyphenyl)-1-(4-fluoro-3-methoxyphenyl)-1H-indazole (145 mg, 0.38 mmol) and DCM (4 mL) was cooled in a dry ice/acetone bath. Boron tribromide (1 M in DCM, 2.0 mL, 2.0 mmol) was added. The reaction was stirred at 0° C. overnight, cooled in a dry ice/acetone bath, quenched with methanol (4 mL), allowed to warm to room temperature, and then concentrated. The residue was triturated in methanol (5 mL) to give 2-chloro-4-(1-(4-fluoro-3-hydroxyphenyl)-1H-indazol-5-yl)phenol (70 mg, 52%) as a white solid. ¹H NMR (400 MHz, DMSO-d₆): δ 10.86-10.17 (m, 1H), 10.16-9.68 (m, 1H), 8.40-8.37 (m, 1H), 7.88-7.79 (m, 2H), 7.51 (dd, J=1.7, 8.8 Hz, 1H), 7.41-7.32 (m, 2H), 7.32-7.27 (m, 1H), 7.26-7.14 (m, 1H), 6.97 (d, J=2.4 Hz, 1H), 6.86 (dd, J=2.4, 8.4 Hz, 1H); LCMS 354.9 [M+H]⁺.

The Compounds below were synthesized in a similar manner to that described for Compound 4.

| Cmpd | Structure | Name | [M + H]⁺ |
|---|---|---|---|
| 4.01 | 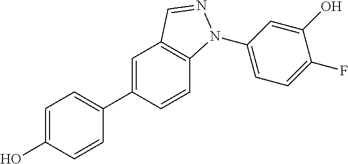 [SMILES: Oc1ccc(-c2ccc3c(cnn3-c3ccc(F)c(O)c3)c2)cc1] | 2-Fluoro-5-(5-(4-hydroxyphenyl)- 1H-indazol-1-yl)phenol | 321.0 |
| 4.02 | 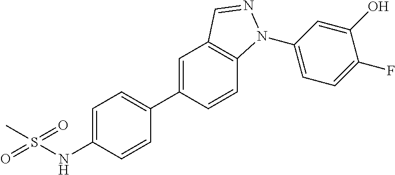 [SMILES: CS(=O)(=O)Nc1ccc(-c2ccc3c(cnn3-c3ccc(F)c(O)c3)c2)cc1] | N-(4-(1-(4-Fluoro-3-hydroxyphenyl)- 1H-indazol-5- yl)phenyl)methanesulfonamide | 398.0 |
| 4.03 | 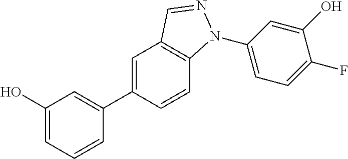 [SMILES: Oc1cccc(-c2ccc3c(cnn3-c3ccc(F)c(O)c3)c2)c1] | 2-Fluoro-5-(5-(3-hydroxyphenyl)- 1H-indazol-1-yl)phenol | 321.0 |
| 4.04 | 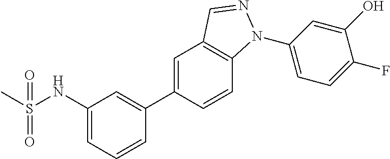 [SMILES: CS(=O)(=O)Nc1cccc(-c2ccc3c(cnn3-c3ccc(F)c(O)c3)c2)c1] | N-(3-(1-(4-Fluoro-3-hydroxyphenyl)- 1H-indazol-5- yl)phenyl)methanesulfonamide | 398.2 |
| 4.05 (1,2) | 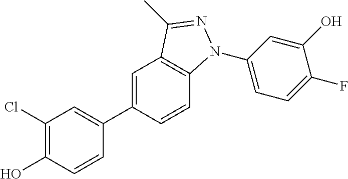 [SMILES: Cc1nn(-c2ccc(F)c(O)c2)c2ccc(-c3ccc(O)c(Cl)c3)cc12] | 2-Chloro-4-(1-(4-fluoro-3- hydroxyphenyl)-3-methyl-1H- indazol-5-yl)phenol | 369.0 |
| 4.06 | 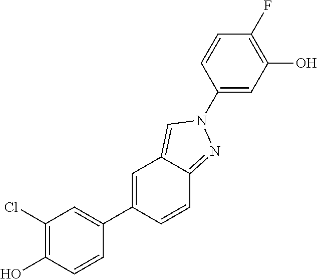 [SMILES: Oc1cc(-n2cc3cc(-c4ccc(O)c(Cl)c4)ccc3n2)ccc1F] | 2-Chloro-4-(2-(4-fluoro-3- hydroxyphenyl)-2H-indazol-5- yl)phenol | 354.9 |
| 4.07 | 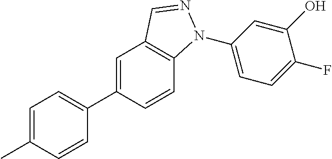 [SMILES: Cc1ccc(-c2ccc3c(cnn3-c3ccc(F)c(O)c3)c2)cc1] | 2-Fluoro-5-(5-(p-tolyl)-1H-indazol-1- yl)phenol | 319.1 |
| 4.08 | 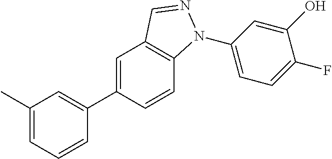 [SMILES: Cc1cccc(-c2ccc3c(cnn3-c3ccc(F)c(O)c3)c2)c1] | 2-Fluoro-5-(5-(m-tolyl)-1H-indazol- 1-yl)phenol | 319.2 |
| 4.09 (3) | 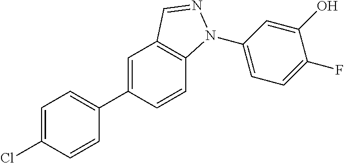 [SMILES: Oc1cc(-n2ncc3cc(-c4ccc(Cl)cc4)ccc32)ccc1F] | 5-(5-(4-Chlorophenyl)-1H-indazol-1- yl)-2-fluorophenol | 338.9 |
| 4.10 (4) | 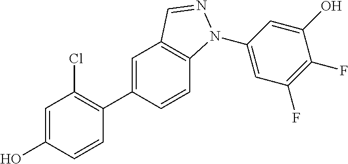 [SMILES: Oc1ccc(-c2ccc3c(cnn3-c3cc(O)c(F)c(F)c3)c2)c(Cl)c1] | 5-(5-(2-Chloro-4-hydroxyphenyl)- 1H-indazol-1-yl)-2,3-difluorophenol | 372.9 |
| 4.11 (3) | 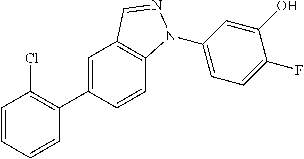 [SMILES: Oc1cc(-n2ncc3cc(-c4ccccc4Cl)ccc32)ccc1F] | 5-(5-(2-Chlorophenyl)-1H-indazol-1- yl)-2-fluorophenol | 339.0 |
| 4.12 (5) | 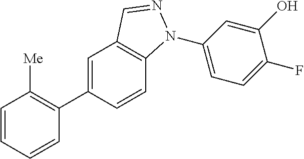 [SMILES: Cc1ccccc1-c1ccc2c(cnn2-c2ccc(F)c(O)c2)c1] | 2-Fluoro-5-(5-(o-tolyl)-1H-indazol-1- yl)phenol | 319.1 |
| 4.13 (6) | 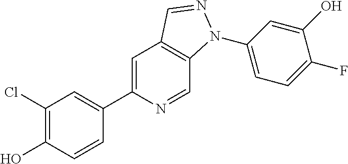 [SMILES: Oc1cc(-n2ncc3cc(-c4ccc(O)c(Cl)c4)ncc32)ccc1F] | 2-Chloro-4-(1-(4-fluoro-3- hydroxyphenyl)-1H-pyrazolo[3,4- c]pyridine-5-yl)phenol | 355.9 |
| 4.14 (3) | 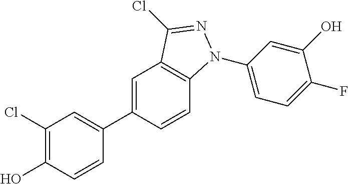 [SMILES: Oc1cc(-n2nc(Cl)c3cc(-c4ccc(O)c(Cl)c4)ccc32)ccc1F] | 2-Chloro-4-(3-chloro-1-(4-fluoro-3- hydroxyphenyl)-1H-indazol-5- yl)phenol | 389.0 |
| 4.15 (3) | 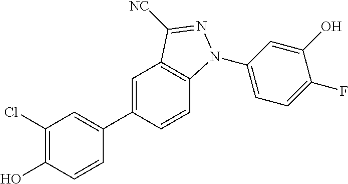 [SMILES: N#Cc1nn(-c2ccc(F)c(O)c2)c2ccc(-c3ccc(O)c(Cl)c3)cc12] | 5-(3-Chloro-4-hydroxyphenyl)-1-(4- fluoro-3-hydroxyphenyl)-1H- indazole-3-carbonitrile | 380.1 |
| 4.16 (4) | 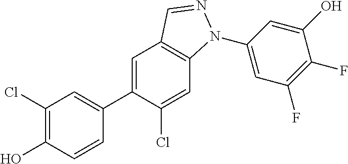 [SMILES: Oc1ccc(-c2cc3cnn(-c4cc(O)c(F)c(F)c4)c3cc2Cl)cc1Cl] | 5-(6-Chloro-5-(3-chloro-4- hydroxyphenyl)-1H-indazol-1-yl)- 2,3-difluorophenol | 406.9 |
| 4.17 (4) | 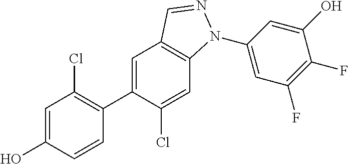 [SMILES: Oc1ccc(-c2cc3cnn(-c4cc(O)c(F)c(F)c4)c3cc2Cl)c(Cl)c1] | 5-(6-Chloro-5-(2-chloro-4- hydroxyphenyl)-1H-indazol-1-yl)- 2,3-difluorophenol | 406.9 |

Alternate conditions used: 1. Step 1: Pd(PPh₃)₄, K₂CO₃, DME:H₂O, 80° C.; 2. Step 2: BBr₃ was added at 0° C.; 3. Step 1: Pd(PPh₃)₄, Cs₂CO₃, DME:H₂O, 80° C.; 4. Step 2: Demethylated following the procedure described for Compound 9, Step 2; 5. Step 1: Pd(PPh₃)₄, Na₂CO₃, DME:H₂O, 90° C., overnight; 6. Step 1: Pd(dppf)Cl₂, Na₂CO₃, CH₃CN:H₂O, 90-120° C. (microwave).

### Compound 5

### 2-Chloro-4-(1-(4-fluoro-3-hydroxyphenyl)-1H-indazol-5-yl)phenol

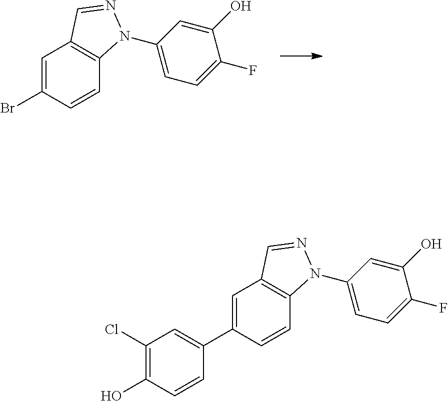 [SMILES: Oc1cc(-n2ncc3cc(Br)ccc32)ccc1F ; Oc1cc(-n2ncc3cc(-c4ccc(O)c(Cl)c4)ccc32)ccc1F]

A mixture of Intermediate 15 (528 mg, 1.72 mmol), 3-chloro-4-hydroxyphenylboronic acid (446 mg, 2.59 mmol), Pd(PPh₃)₄ (201 mg, 0.17 mmol), 2 M Na₂CO₃ (1.8 mL, 3.6 mmol), and dioxane (5 mL) was heated at 80° C. for 1 h, allowed to cool to room temperature, diluted (75 mL EtOAc and 50 mL water), and then filtered. The layers were separated, the organic layer was washed (50 mL brine), dried (Na₂SO₄), and then concentrated. The residue was purified by silica gel chromatography (0-25% EtOAc/hexanes) to give 2-chloro-4-(1-(4-fluoro-3-hydroxyphenyl)-1H-indazol-5-yl)phenol (300 mg, 49%) as a white solid. ¹H NMR (400 MHz, DMSO-d₆): δ 10.39 (s, 1H), 10.30 (s, 1H), 8.37 (s, 1H), 8.10-8.07 (m, 1H), 7.86-7.74 (m, 2H), 7.71 (d, J=2.3 Hz, 1H), 7.54 (dd, J=2.3, 8.5 Hz, 1H), 7.40-7.33 (m, 2H), 7.20 (td, J=3.2, 8.7 Hz, 1H), 7.08 (d, J=8.6 Hz, 1H); LCMS 354.9 [M+H]⁺.

The Compounds below were synthesized in a similar manner to that described for Compound 5.

| Cmpd | Structure | Name | [M + H]⁺ |
|---|---|---|---|
| 5.01 | 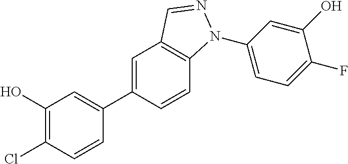 [SMILES: Oc1cc(-n2ncc3cc(-c4ccc(Cl)c(O)c4)ccc32)ccc1F] | 2-Chloro-5-(1-(4-fluoro-3- hydroxyphenyl)-1H-indazol-5- yl)phenol | 354.9 |
| 5.02 (1,2) | 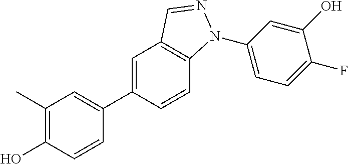 [SMILES: Cc1cc(-c2ccc3c(cnn3-c3ccc(F)c(O)c3)c2)ccc1O] | 4-(1-(4-Fluoro-3-hydroxyphenyl)- 1H-indazol-5-yl)-2-methylphenol | 334.9 |
| 5.03 | 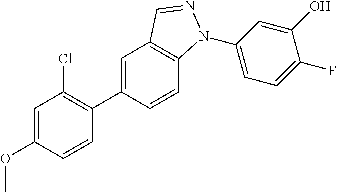 [SMILES: COc1ccc(-c2ccc3c(cnn3-c3ccc(F)c(O)c3)c2)c(Cl)c1] | 5-(5-(2-Chloro-4-methoxyphenyl)- 1H-indazol-1-yl)-2-fluorophenol | 368.9 |
| 5.04 (7) | 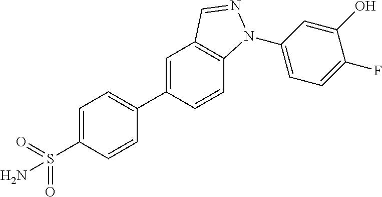 [SMILES: NS(=O)(=O)c1ccc(-c2ccc3c(cnn3-c3ccc(F)c(O)c3)c2)cc1] | 4-(1-(4-Fluoro-3-hydroxyphenyl)- 1H-indazol-5-yl)benzenesulfonamide | 384.0 |
| 5.05 | 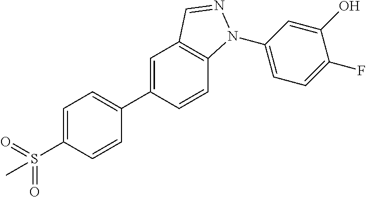 [SMILES: CS(=O)(=O)c1ccc(-c2ccc3c(cnn3-c3ccc(F)c(O)c3)c2)cc1] | 2-Fluoro-5-(5-(4- (methylsulfonyl)phenyl)-1H-indazol- 1-yl)phenol | 383.0 |
| 5.06 | 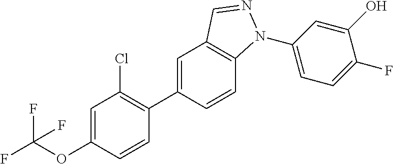 [SMILES: Oc1cc(-n2ncc3cc(-c4ccc(OC(F)(F)F)cc4Cl)ccc32)ccc1F] | 5-(5-(2-Chloro-4- (trifluoromethoxy)phenyl)-1H- indazol-1-yl)-2-fluorophenol | 423.0 |
| 5.07 | 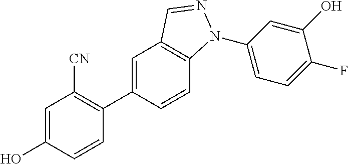 [SMILES: N#Cc1cc(O)ccc1-c1ccc2c(cnn2-c2ccc(F)c(O)c2)c1] | 2-(1-(4-Fluoro-3-hydroxyphenyl)- 1H-indazol-5-yl)-5- hydroxybenzonitrile | 346.0 |
| 5.08 | 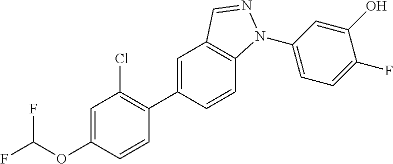 [SMILES: Oc1cc(-n2ncc3cc(-c4ccc(OC(F)F)cc4Cl)ccc32)ccc1F] | 5-(5-(2-Chloro-4- (difluoromethoxy)phenyl)-1H- indazol-1-yl)-2-fluorophenol | 405.0 |
| 5.09 | 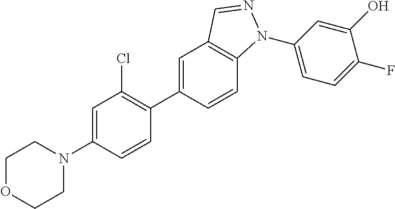 [SMILES: Oc1cc(-n2ncc3cc(-c4ccc(N5CCOCC5)cc4Cl)ccc32)ccc1F] | 5-(5-(2-Chloro-4-morpholinophenyl)- 1H-indazol-1-yl)-2-fluorophenol | 424.1 |
| 5.10 | 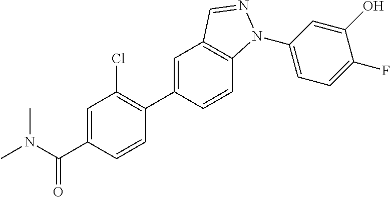 [SMILES: CN(C)C(=O)c1ccc(-c2ccc3c(cnn3-c3ccc(F)c(O)c3)c2)c(Cl)c1] | 3-Chloro-4-(1-(4-fluoro-3- hydroxyphenyl)-1H-indazol-5-yl)- N,N-dimethylbenzamide | 410.1 |
| 5.11 | 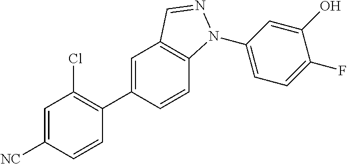 [SMILES: N#Cc1ccc(-c2ccc3c(cnn3-c3ccc(F)c(O)c3)c2)c(Cl)c1] | 3-Chloro-4-(1-(4-fluoro-3- hydroxyphenyl)-1H-indazol-5- yl)benzonitrile | 364.0 |
| 5.12 | 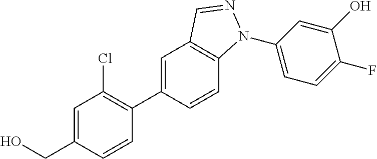 [SMILES: OCc1ccc(-c2ccc3c(cnn3-c3ccc(F)c(O)c3)c2)c(Cl)c1] | 5-(5-(2-Chloro-4- (hydroxymethyl)phenyl)-1H-indazol- 1-yl)-2-fluorophenol | 369.0 |
| 5.13 | 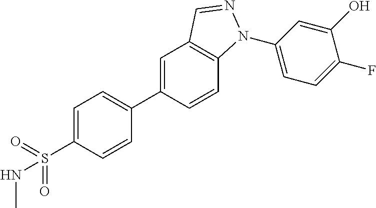 [SMILES: CNS(=O)(=O)c1ccc(-c2ccc3c(cnn3-c3ccc(F)c(O)c3)c2)cc1] | 4-(1-(4-Fluoro-3-hydroxyphenyl)- 1H-indazol-5-yl)-N- methylbenzenesulfonamide | 398.0 |
| 5.14 | 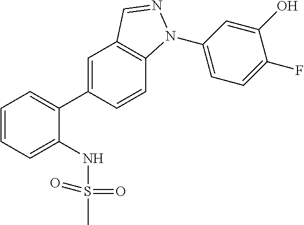 [SMILES: CS(=O)(=O)Nc1ccccc1-c1ccc2c(cnn2-c2ccc(F)c(O)c2)c1] | N-(2-(1-(4-Fluoro-3-hydroxyphenyl)- 1H-indazol-5- yl)phenyl)methanesulfonamide | 398.2 |
| 5.15 | 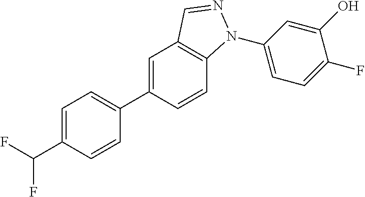 [SMILES: Oc1cc(-n2ncc3cc(-c4ccc(C(F)F)cc4)ccc32)ccc1F] | 5-(5-(4-(Difluoromethyl)phenyl)-1H- indazol-1-yl)-2-fluorophenol | 355.0 |
| 5.16 | 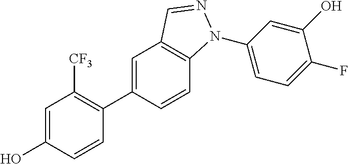 [SMILES: Oc1ccc(-c2ccc3c(cnn3-c3ccc(F)c(O)c3)c2)c(C(F)(F)F)c1] | 2-Fluoro-5-(5-(4-hydroxy-2- (trifluoromethyl)phenyl)-1H-indazol- 1-yl)phenol | 389.1 |
| 5.17 | 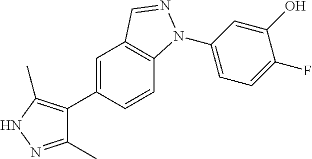 [SMILES: Cc1n[nH]c(C)c1-c1ccc2c(cnn2-c2ccc(F)c(O)c2)c1] | 5-(5-(3,5-Dimethyl-1H-pyrazol-4-yl)- 1H-indazol-1-yl)-2-fluorophenol | 323.3 |
| 5.18 | 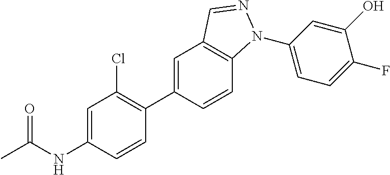 [SMILES: CC(=O)Nc1ccc(-c2ccc3c(cnn3-c3ccc(F)c(O)c3)c2)c(Cl)c1] | N-(3-Chloro-4-(1-(4-fluoro-3- hydroxyphenyl)-1H-indazol-5- yl)phenyl)acetamide | 396.1 |
| 5.19 | 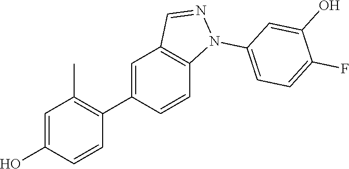 [SMILES: Cc1cc(O)ccc1-c1ccc2c(cnn2-c2ccc(F)c(O)c2)c1] | 2-Fluoro-5-(5-(4-hydroxy-2- methylphenyl)-1H-indazol-1- yl)phenol | 335.1 |
| 5.20 (2) | 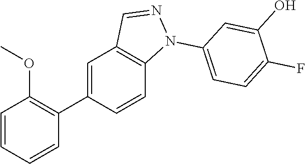 [SMILES: COc1ccccc1-c1ccc2c(cnn2-c2ccc(F)c(O)c2)c1] | 2-Fluoro-5-(5-(2-methoxyphenyl)- 1H-indazol-1-yl)phenol | 335.1 |
| 5.21 (4) | 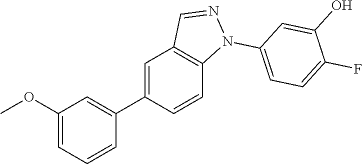 [SMILES: COc1cccc(-c2ccc3c(cnn3-c3ccc(F)c(O)c3)c2)c1] | 2-Fluoro-5-(5-(3-methoxyphenyl)- 1H-indazol-1-yl)phenol | 335.0 |
| 5.22 (2) | 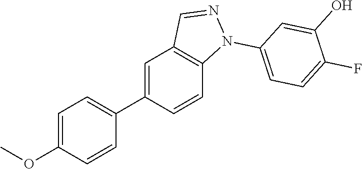 [SMILES: COc1ccc(-c2ccc3c(cnn3-c3ccc(F)c(O)c3)c2)cc1] | 2-Fluoro-5-(5-(4-methoxyphenyl)- 1H-indazol-1-yl)phenol | 334.9 |
| 5.23 (2,3) | 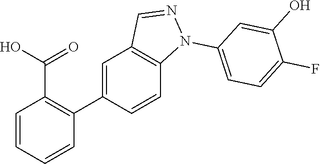 [SMILES: O=C(O)c1ccccc1-c1ccc2c(cnn2-c2ccc(F)c(O)c2)c1] | 2-(1-(4-Fluoro-3-hydroxyphenyl)- 1H-indazol-5-yl)benzoic acid | 348.9 |
| 5.24 (2,3) | 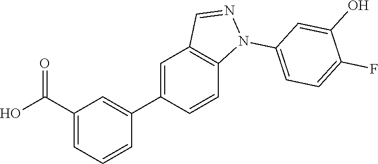 [SMILES: O=C(O)c1cccc(-c2ccc3c(cnn3-c3ccc(F)c(O)c3)c2)c1] | 3-(1-(4-Fluoro-3-hydroxyphenyl)- 1H-indazol-5-yl)benzoic acid | 348.9 |
| 5.25 (1) | 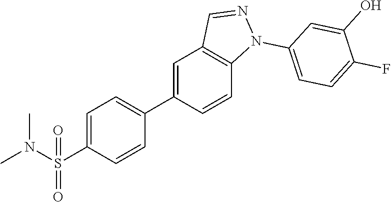 [SMILES: CN(C)S(=O)(=O)c1ccc(-c2ccc3c(cnn3-c3ccc(F)c(O)c3)c2)cc1] | 4-(1-(4-Fluoro-3-hydroxyphenyl)- 1H-indazol-5-yl)-N,N- dimethylbenzenesulfonamide | 412.0 |
| 5.26 | 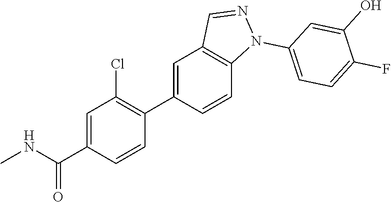 [SMILES: CNC(=O)c1ccc(-c2ccc3c(cnn3-c3ccc(F)c(O)c3)c2)c(Cl)c1] | 3-Chloro-4-(1-(4-fluoro-3- hydroxyphenyl)-1H-indazol-5-yl)-N- methylbenzamide | 396.0 |
| 5.27 (5) | 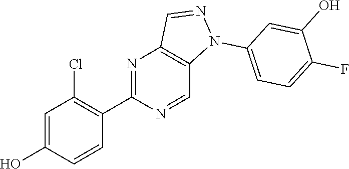 [SMILES: Oc1ccc(-c2ncc3c(cnn3-c3ccc(F)c(O)c3)n2)c(Cl)c1] | 5-(5-(2-Chloro-4-hydroxyphenyl)- 1H-pyrazolo[4,3-d]pyrimidin-1-yl)-2- fluorophenol | 357.0 |
| 5.28 (5) | 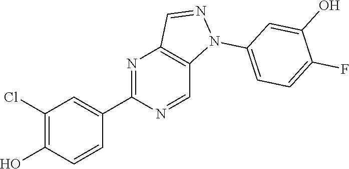 [SMILES: Oc1cc(-n2ncc3nc(-c4ccc(O)c(Cl)c4)ncc32)ccc1F] | 2-Chloro-4-(1-(4-fluoro-3- hydroxyphenyl)-1H-pyrazolo[4,3- d]pyrimidin-5-yl)phenol | 357.0 |
| 5.29 (6,3) | 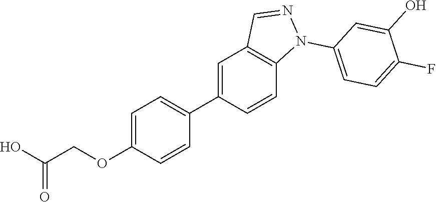 [SMILES: O=C(O)COc1ccc(-c2ccc3c(cnn3-c3ccc(F)c(O)c3)c2)cc1] | 2-(4-(1-(4-Fluoro-3-hydroxyphenyl)- 1H-indazol-5-yl)phenoxy)acetic acid | 377.1 [M − H]⁻ |
| 5.30 | 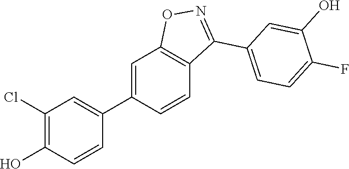 [SMILES: Oc1cc(-c2noc3cc(-c4ccc(O)c(Cl)c4)ccc23)ccc1F] | 2-Chloro-4-(3-(4-fluoro-3- hydroxyphenyl)benzo[d]isoxazol-6- yl)phenol | 355.9 |

Alternate conditions used: 1. Pd(dppf)Cl₂ K₂CO₃, dioxane:H₂O, 80° C.; 2. Pd(PPh₃)₄, Cs₂CO₃, DME:H₂O 80° C.; 3. From carboxylic ester hydrolysis (LiOH in THF:MeOH:H₂O); 4. Pd(PPh₃)₄, Cs₂CO₃, DME:H₂O, 80° C.; 5. Pd(dppf)C₁₂, Na₂CO₃, CH₃CN:H₂O, 90-120° C. (microwave); 6. Pd₂(dba)₃, PCy₃, K₃PO₄, dioxane:H₂O, 100° C.; 7. Pd₂(dba)₃, Xphos, K₃PO₄, dioxane:H₂O, 100° C.

### Compound 6

### 2-(1-(4-Fluoro-3-hydroxyphenyl)-1H-indazol-5-yl)benzonitrile

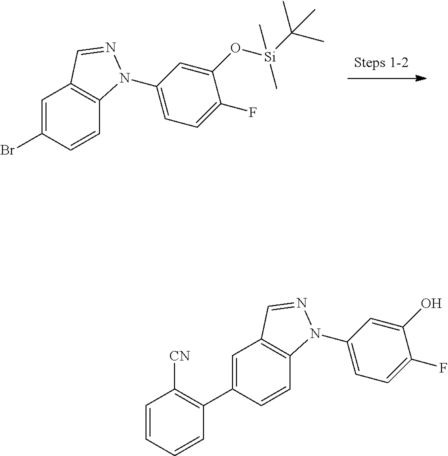 [SMILES: CC(C)(C)[Si](C)(C)Oc1cc(-n2ncc3cc(Br)ccc32)ccc1F ; N#Cc1ccccc1-c1ccc2c(cnn2-c2ccc(F)c(O)c2)c1]

### Step 1: 2-(1-(3-((tert-Butyldimethylsilyl)oxy)-4-fluorophenyl)-1H-indazol-5-yl)benzonitrile

Pd(PPh₃)₄ (21 mg, 0.018 mmol) was added to a mixture of Intermediate 16 (150 mg 0.356 mmol), (2-cyanophenyl)boronic acid (60 mg, 0.410 mmol), and Na₂CO₃ (2 M, 0.5 mL) in dioxane (3 mL) under N₂. The mixture was stirred at 90° C. for 16 h, filtered, and then concentrated to give 2-(1-(3-((tert-butyldimethylsilyl)oxy)-4-fluorophenyl)-1H-indazol-5-yl)benzonitrile (100 mg).

### Step 2: 2-(1-(4-Fluoro-3-hydroxyphenyl)-1H-indazol-5-yl)benzonitrile

Ammonium fluoride (132 mg, 3.56 mmol) was added to a solution of 2-(1-(3-((tert-butyldimethylsilyl)oxy)-4-fluorophenyl)-1H-indazol-5-yl)benzonitrile (100 mg) in MeOH (3 mL). The mixture was stirred at 80° C. for 1 h, poured into H₂O (10 mL), and then extracted (3×10 mL EtOAc). The combined organic layers were washed (2×10 mL brine), dried (Na₂SO₄), filtered, and then concentrated. The residue was purified by silica gel chromatography (10-40% EtOAc/petroleum ether) to give 2-(1-(4-fluoro-3-hydroxyphenyl)-1H-indazol-5-yl)benzonitrile (15 mg, 13%) as a white solid. ¹H NMR (400 MHz, DMSO-d₆): δ 10.39 (s, 1H), 8.47 (s, 1H), 8.11 (s, 1H), 7.98 (d, 1H), 7.92 (d, 1H), 7.80-7.84 (m, 1H), 7.68-7.71 (m, 2H), 7.58-7.62 (m, 1H), 7.34-7.39 (m, 2H), 7.21-7.24 (m, 1H); LCMS: 330.1 [M+H]⁺.

The Compound below was synthesized in a similar manner to that described for Compound 6.

| Cmpd | Structure | Name | [M + H]⁺ |
|---|---|---|---|
| 6.01 | 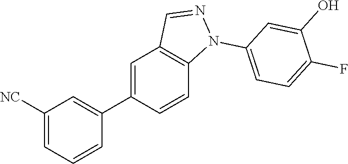 [SMILES: N#Cc1cccc(-c2ccc3c(cnn3-c3ccc(F)c(O)c3)c2)c1] | 3-(1-(4-Fluoro-3-hydroxyphenyl)- 1H-indazol-5-yl)benzonitrile | 330.0 |

### Compound 7

### 5-(5-(5-Chloropyridin-3-yl)-1H-indazol-1-yl)-2,3-difluorophenol

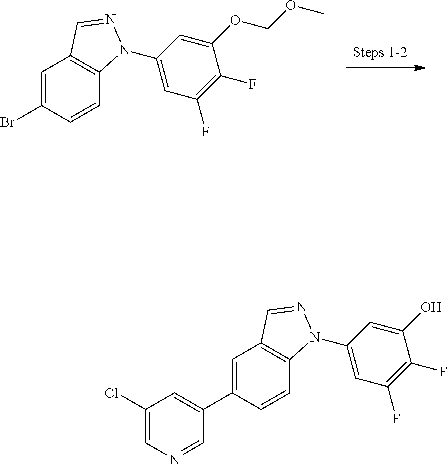 [SMILES: COCOc1cc(-n2ncc3cc(Br)ccc32)cc(F)c1F ; Oc1cc(-n2ncc3cc(-c4cncc(Cl)c4)ccc32)cc(F)c1F]

### Step 1: 5-(5-Chloropyridin-3-yl)-1-(3,4-difluoro-5-(methoxymethoxy)phenyl)-1H-indazole

Pd(dppf)Cl₂·CH₂Cl₂ (16 mg, 0.018 mmol) was added to a mixture of Intermediate 18 (200 mg, 0.38 mmol), (5-chloropyridin-3-yl)boronic acid (66 mg, 0.42 mmol), Na₂CO₃ (3.6 M, 0.4 mL, 1.44 mmol), and dioxane (2 mL)under N₂. The mixture was degassed and purged with N₂ three times, heated at 100° C. for 4 h, allowed to cool to room temperature, and then filtered through a Celite pad. The filtrate was poured into water (10 mL) and extracted (2×10 mL EtOAc). The combined organics were washed (10 mL brine), dried (Na₂SO₄), filtered, and then concentrated. The residue was purified by silica gel chromatography (5-20% EtOAc/petroleum ether) to give 5-(5-chloropyridin-3-yl)-1-(3,4-difluoro-5-(methoxymethoxy)phenyl)-1H-indazole (130 mg, 85%) as a red solid. ¹H NMR (400 MHz, DMSO-d₆): δ 8.94 (d, 1H), 8.62 (d, 1H), 8.48 (s, 1H), 8.33-8.31 (m, 2H), 8.00-7.92 (m, 2H), 7.58-7.52 (m, 2H), 5.43 (s, 2H), 3.48 (s, 3H); LCMS: 402.0 [M+H]⁺.

### Step 2: 5-(5-(5-Chloropyridin-3-yl)-1H-indazol-1-yl)-2,3-difluorophenol

Aqueous hydrochloric acid (3 M, 1.8 mL, 5.4 mmol) was added to a solution of 5-(5-chloropyridin-3-yl)-1-(3,4-difluoro-5-(methoxymethoxy)phenyl)-1H-indazole (180 mg, 0.448 mmol) in MeOH (1 mL) and THF (1 mL). The mixture was heated at 90° C. for 0.5 h and allowed to cool to room temperature. The pH was adjusted with saturated NaHCO₃ to pH-8, and the mixture was extracted (3×10 mL EtOAc). The combined organic phases were washed (2×10 mL brine), dried (Na₂SO₄), filtered, and then concentrated. The residue was purified by prep-HPLC [water (0.04% NH₃H₂O+10 mM NH₄HCO₃)-ACN], lyophilized, and then dissolved in DCM (5 mL). The solution was washed (5 mL saturated ammonium chloride solution), and the aqueous phase was extracted (3×5 mL DCM). The combined organic phases were dried (Na₂SO₄), filtered, and then concentrated to give 5-(5-(5-chloropyridin-3-yl)-1H-indazol-1-yl)-2,3-difluorophenol (36 mg, 22%) as a yellow solid. ¹H NMR (400 MHz, DMSO-d₆): δ 10.95 (s, 1H), 8.95 (s, 1H), 8.63 (s, 1H), 8.46 (s, 1H), 8.33 (s, 2H), 7.98-7.93 (m, 2H), 7.32-7.31 (m, 1H), 7.26-7.24 (m, 1H); LCMS: 358.0 [M+H]⁺.

The Compounds below were synthesized in a similar manner to that described for Compound 7.

| Cmpd | Structure | Name | [M + H]⁺ |
|---|---|---|---|
| 7.01 | 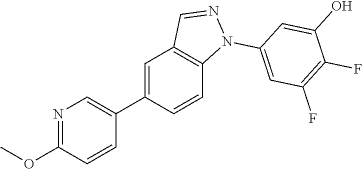 [SMILES: COc1ccc(-c2ccc3c(cnn3-c3cc(O)c(F)c(F)c3)c2)cn1] | 2,3-Difluoro-5-(5-(6- methoxypyridin-3-yl)-1H-indazol-1- yl)phenol | 354.2 |
| 7.02 | 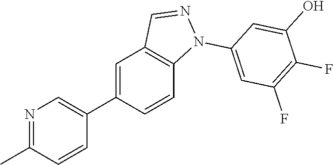 [SMILES: Cc1ccc(-c2ccc3c(cnn3-c3cc(O)c(F)c(F)c3)c2)cn1] | 2,3-Difluoro-5-(5-(6-methylpyridin- 3-yl)-1H-indazol-1-yl)phenol | 338.1 |
| 7.03 | 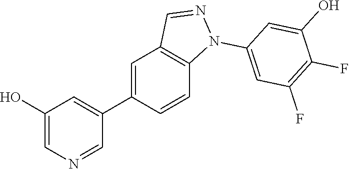 [SMILES: Oc1cncc(-c2ccc3c(cnn3-c3cc(O)c(F)c(F)c3)c2)c1] | 5-(1-(3,4-Difluoro-5- hydroxyphenyl)-1H-indazol-5- yl)pyridine-3-ol | 340.1 |
| 7.04 | 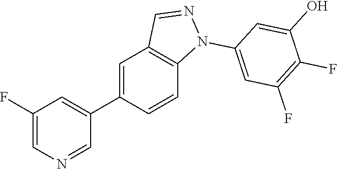 [SMILES: Oc1cc(-n2ncc3cc(-c4cncc(F)c4)ccc32)cc(F)c1F] | 2,3-Difluoro-5-(5-(5-fluoropyridin-3- yl)-1H-indazol-1-yl)phenol | 342.0 |

### Compound 8

### 3-(5-(3-Chloro-4-hydroxyphenyl)-1H-indazol-1-yl)-2,6-difluorophenol

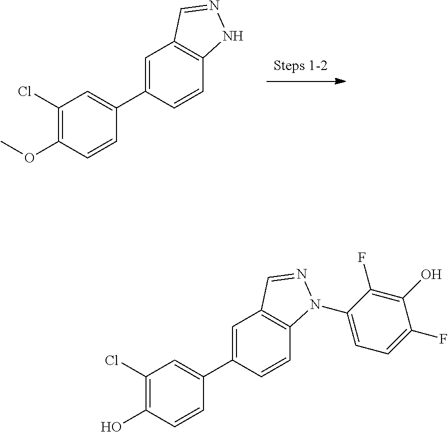 [SMILES: COc1ccc(-c2ccc3[nH]ncc3c2)cc1Cl ; Oc1ccc(-c2ccc3c(cnn3-c3ccc(F)c(O)c3F)c2)cc1Cl]

### Step 1: 5-(3-Chloro-4-methoxyphenyl)-1-(2,4-difluoro-3-methoxyphenyl)-1H-indazole

A mixture of Intermediate 10 (250 mg, 0.97 mmol), 4-bromo-1,3-difluoro-2-methoxybenzene (431 mg, 1.93 mmol), t-BuXPhos (86 mg, 0.20 mmol), Pd₂(dba)₃ (46 mg, 0.06 mmol), sodium tert-butoxide (141 mg, 1.46 mmol), and toluene (3 mL) was heated at 100° C. overnight. Additional t-BuXPhos (170 mg, 0.40 mmol) and Pd₂(dba)₃ (90 mg, 0.10 mmol) were added to the reaction, and the mixture was heated at 100° C. for an additional 2 h. The reaction was diluted (20 mL EtOAc and 20 mL brine) and filtered through a Celite plug. The layers were separated. The organic layer was dried (Na₂SO₄) and concentrated. The residue was purified by silica gel chromatography (0-15% EtOAc/heptane) to give 5-(3-chloro-4-methoxyphenyl)-1-(2,4-difluoro-3-methoxyphenyl)-1H-indazole (60 mg, 21%). ¹H NMR (400 MHz, DMSO-d₆) δ 8.45 (d, J=0.7 Hz, 1H), 8.15 (d, J=1.0 Hz, 1H), 7.84-7.75 (m, 2H), 7.70 (dd, J=2.3, 8.6 Hz, 1H), 7.56-7.50 (m, 1H), 7.50-7.45 (m, 1H), 7.44-7.37 (m, 1H), 7.27 (d, J=8.7 Hz, 1H), 4.06 (s, 3H), 3.92 (s, 3H); LCMS 401.0 [M+H]⁺.

### Step 2: 3-(5-(3-Chloro-4-hydroxyphenyl)-1H-indazol-1-yl)-2,6-difluorophenol

A mixture of 5-(3-chloro-4-methoxyphenyl)-1-(2,4-difluoro-3-methoxyphenyl)-1H-indazole (70 mg, 0.17 mmol) and pyridinium chloride (745 mg, 6.45 mmol)) was heated at 180° C. for 5 h and then cooled to room temperature. The reaction was diluted (4 mL of 1 N hydrochloric acid, 20 mL EtOAc, and then 15 mL water). The layers were separated. The organic layer was washed (20 mL brine), dried (Na₂SO₄), and then concentrated. The residue was purified by prep-HPLC to give 3-(5-(3-chloro-4-hydroxyphenyl)-1H-indazol-1-yl)-2,6-difluorophenol (45 mg, 60%) as an off-white solid. ¹H NMR (400 MHz, DMSO-d₆): δ 10.80 (s, 1H), 10.30 (s, 1H), 8.41 (d, J=0.7 Hz, 1H), 8.10-8.07 (m, 1H), 7.73 (dd, J=1.7, 8.8 Hz, 1H), 7.69 (d, J=2.3 Hz, 1H), 7.52 (dd, J=2.3, 8.4 Hz, 1H), 7.43 (dd, J=2.7, 8.8 Hz, 1H), 7.31-7.24 (m, 1H), 7.14 (dt, J=5.4, 8.4 Hz, 1H), 7.08 (d, J=8.4 Hz, 1H); LCMS: 373.0 [M+H]⁺.

The Compounds below were synthesized in a similar manner to that described for Compound 8.

| Cmpd | Structure | Name | [M + H]⁺ |
|---|---|---|---|
| 8.01 | 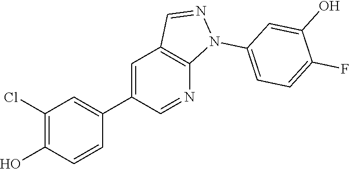 [SMILES: Oc1cc(-n2ncc3cc(-c4ccc(O)c(Cl)c4)cnc32)ccc1F] | 2-Chloro-4-(1-(4-fluoro-3- hydroxyphenyl)-1H-pyrazolo[3,4- b]pyridine-5-yl)phenol | 355.9 |
| 8.02 | 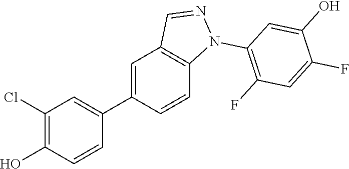 [SMILES: Oc1cc(-n2ncc3cc(-c4ccc(O)c(Cl)c4)ccc32)c(F)cc1F] | 5-(5-(3-Chloro-4-hydroxyphenyl)- 1H-indazol-1-yl)-2,4-difluorophenol | 372.9 |
| 8.03 | 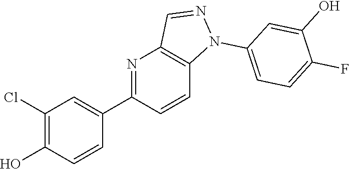 [SMILES: Oc1cc(-n2ncc3nc(-c4ccc(O)c(Cl)c4)ccc32)ccc1F] | 2-Chloro-4-(1-(4-fluoro-3- hydroxyphenyl)-1H-pyrazolo[4,3- b]pyridine-5-yl)phenol | 355.9 |

### Compound 9

### 5-(5-(4-Amino-2-chlorophenyl)-1H-indazol-1-yl)-2-fluorophenol

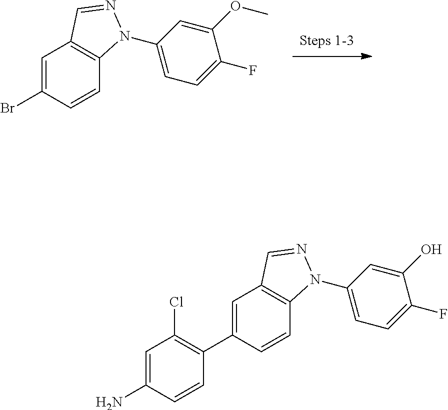 [SMILES: COc1cc(-n2ncc3cc(Br)ccc32)ccc1F ; Nc1ccc(-c2ccc3c(cnn3-c3ccc(F)c(O)c3)c2)c(Cl)c1]

### Step 1: tert-Butyl (3-chloro-4-(1-(4-fluoro-3-methoxyphenyl)-1H-indazol-5-yl)phenyl)carbamate

tert-Butyl (3-chloro-4-(1-(4-fluoro-3-methoxyphenyl)-1H-indazol-5-yl)phenyl)carbamate was synthesized from Intermediate 14 and (4-((tert-butoxycarbonyl)amino)-2-chlorophenyl)boronic acid in a similar manner to the synthesis of Compound 4, Step 1. ¹H NMR (400 MHz, DMSO-d₆): δ 9.68 (s, 1H), 8.43 (s, 1H), 7.90 (dd, J=3.6, 5.2 Hz, 2H), 7.77 (d, J=1.8 Hz, 1H), 7.53 (ddd, J=2.1, 4.6, 8.2 Hz, 2H), 7.50-7.42 (m, 2H), 7.41-7.31 (m, 2H), 3.96 (s, 3H), 1.51 (s, 9H); LCMS 468.1 [M+H]⁺.

### Step 2: 3-Chloro-4-(1-(4-fluoro-3-methoxyphenyl)-1H-indazol-5-yl)aniline

Trifluoroacetic acid (1 mL) was added to a mixture of tert-butyl (3-chloro-4-(1-(4-fluoro-3-hydroxyphenyl)-1H-indazol-5-yl)phenyl)carbamate (375 mg, 0.80 mmol) and DCM (4 mL). The mixture was stirred for 2 h and concentrated. The residue was dissolved in DCM (20 mL), washed (20 mL saturated NaHCO₃ and then 20 mL brine), dried (Na₂SO₄), and then concentrated to give 3-chloro-4-(1-(4-fluoro-3-methoxyphenyl)-1H-indazol-5-yl)aniline (268 mg, 910%) as a yellow foam. ¹H NMR (400 MHz, DMSO-d₆): δ 8.39 (d, J=0.6 Hz, 1H), 7.86 (d, J=8.8 Hz, 1H), 7.82 (d, J=1.0 Hz, 1H), 7.53 (dd, J=2.4, 7.7 Hz, 1H), 7.49 (dd, J 1.7, 8.7 Hz, 1H), 7.44 (dd, J=8.7, 11.2 Hz, 1H), 7.37-7.31 (m, 1H), 7.13 (d, J=8.3 Hz, 1H), 6.75 (d, J=2.3 Hz, 1H), 6.62 (dd, J=2.2, 8.3 Hz, 1H), 5.75-5.20 (m, 2H), 3.96 (s, 3H); LCMS 368.1 [M+H]⁺.

### Step 3: 5-(5-(4-Amino-2-chlorophenyl)-1H-indazol-1-yl)-2-fluorophenol

5-(5-(4-Amino-2-chlorophenyl)-1H-indazol-1-yl)-2-fluorophenol was synthesized in a similar manner to that described for Compound 8, Step 2. ¹H NMR (400 MHz, DMSO-d₆): δ 10.37 (br s, 1H), 8.37-8.35 (m, 1H), 7.83-7.77 (m, 2H), 7.49 (dd, J=1.7, 8.7 Hz, 1H), 7.39-7.33 (m, 2H), 7.26-7.18 (m, 1H), 7.14 (d, J=8.3 Hz, 1H), 6.76 (d, J=2.2 Hz, 1H), 6.64 (dd, J=2.2, 8.3 Hz, 1H), 6.28-4.96 (m, 2H); LCMS 354.1 [M+H]⁺.

### Compound 10

### N-(3-Chloro-4-(1-(4-fluoro-3-hydroxyphenyl)-1H-indazol-5-yl)phenyl)methanesulfonamide

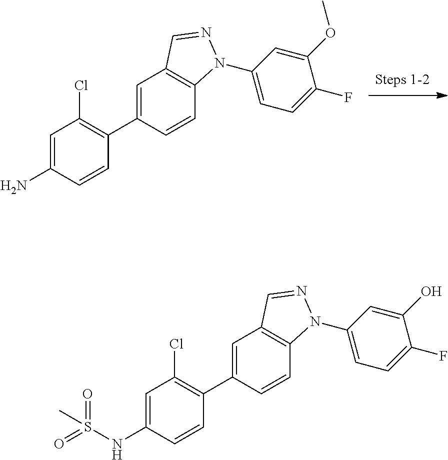 [SMILES: COc1cc(-n2ncc3cc(-c4ccc(N)cc4Cl)ccc32)ccc1F ; CS(=O)(=O)Nc1ccc(-c2ccc3c(cnn3-c3ccc(F)c(O)c3)c2)c(Cl)c1]

### Step 1: N-(3-Chloro-4-(1-(4-fluoro-3-methoxyphenyl)-1H-indazol-5-yl)phenyl)methanesulfonamide

Methanesulfonyl chloride (39 μL, 0.50 mmol) was added to a mixture of Compound 9, Step 2 (117 mg, 0.32 mmol), triethylamine (0.1 mL, 0.72 mmol), and DCM (10 mL). The mixture was stirred for 3 h, diluted (10 mL DCM), washed (20 mL saturated NaHCO₃ and then 20 mL brine), dried (Na₂SO₄), and then concentrated. The residue was dissolved in THE (3 mL). 1 M TBAF in THF (3 mL) was added. The mixture was stirred for 3 h, diluted (20 mL EtOAc), washed (15 mL saturated NaHCO₃ and then 20 mL brine), dried (Na₂SO₄), and then concentrated. The residue was purified by silica gel chromatography (20-40% EtOAc/hexanes) to give N-(3-chloro-4-(1-(4-fluoro-3-methoxyphenyl)-1H-indazol-5-yl)phenyl)methanesulfonamide (113 mg, 80%) as a white solid. ¹H NMR (400 MHz, DMSO-d₆): δ 10.12 (s, 1H), 8.44 (s, 1H), 7.94-7.90 (m, 2H), 7.56-7.52 (m, 2H), 7.50-7.42 (m, 2H), 7.40 (d, J=2.2 Hz, 1H), 7.38-7.32 (m, 1H), 7.29 (dd, J=2.3, 8.4 Hz, 1H), 3.96 (s, 3H), 3.12 (s, 3H); LCMS 446.0 [M+H]⁺.

### Step 2: N-(3-Chloro-4-(1-(4-fluoro-3-hydroxyphenyl)-1H-indazol-5-yl)phenyl)methanesulfonamide

N-(3-Chloro-4-(1-(4-fluoro-3-hydroxyphenyl)-1H-indazol-5-yl)phenyl)methanesulfonamide was synthesized in a similar manner to that described for Compound 4, Step 2. ¹H NMR (400 MHz, DMSO-d₆): δ 10.39 (s, 1H), 10.12 (s, 1H), 8.41 (d, J=0.6 Hz, 1H), 7.91 (d, J=0.9 Hz, 1H), 7.85 (d, J=8.8 Hz, 1H), 7.54 (dd, J=1.6, 8.8 Hz, 1H), 7.48 (d, J=8.3 Hz, 1H), 7.43-7.33 (m, 3H), 7.29 (dd, J=2.2, 8.4 Hz, 1H), 7.25-7.18 (m, 1H), 3.11 (s, 3H); LCMS 432.0 [M+H]⁺.

### Compound 11

### N-(4-(1-(4-Fluoro-3-hydroxyphenyl)-1H-indazol-5-yl)phenyl)propane-2-sulfonamide

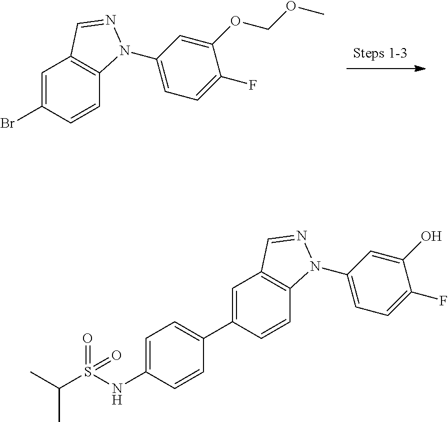

### Step 1: 4-(1-(4-Fluoro-3-(methoxymethoxy)phenyl)-1H-indazol-5-yl)aniline

Pd(dppf)Cl₂ (125 mg, 0.17 mmol) was added to a mixture of Intermediate 17 (1.2 g), 4-(4,4,5,5-tetramethyl-1,3,2-dioxaborolan-2-yl)aniline (786 mg, 3.59 mmol), Cs₂CO₃ (3.34 g, 10.25 mmol), and dioxane (10 mL) at room temperature. The mixture was degassed with 3 vacuum/N₂ cycles, stirred at 100° C. overnight, allowed to cool to room temperature, poured into H₂O (50 mL), and then extracted (3×50 mL EtOAc). The combined organic layers were washed (100 mL brine), dried (Na₂SO₄), filtered, concentrated, and then purified by silica gel chromatography (50% EtOAc/petroleum ether) to give 4-(1-(4-fluoro-3-(methoxymethoxy)phenyl)-1H-indazol-5-yl)aniline (900 mg, 72%) as a yellow solid. ¹H NMR (400 MHz, DMSO-d₆): δ 8.35 (d, 1H), 7.97 (d, 1H), 7.84-7.79 (m, 1H), 7.71 (dd, 1H), 7.64 (dd, 1H), 7.50-7.39 (m, 4H), 6.67 (d, 2H), 5.37 (s, 2H), 5.19 (s, 2H), 3.46 (s, 3H); LCMS: 364.2 [M+H]⁺.

### Step 2: N-(4-(1-(4-Fluoro-3-(methoxymethoxy)phenyl)-1H-indazol-5-yl)phenyl)propane-2-sulfonamide

Propane-2-sulfonyl chloride (86 mg, 0.61 mmol) was added to a mixture of 4-(1-(4-fluoro-3-(methoxymethoxy)phenyl)-1H-indazol-5-yl)aniline (200 mg, 0.55 mmol) and pyridine (2 mL) at room temperature. The mixture was stirred for 2 h, poured into saturated NaHCO₃ (50 mL), and then extracted (3×50 mL EtOAc). The combined organic layers were washed (100 mL brine), dried (Na₂SO₄), filtered, and concentrated to give N-(4-(1-(4-fluoro-3-(methoxymethoxy)phenyl)-1H-indazol-5-yl)phenyl)propane-2-sulfonamide (300 mg) as a yellow oil. LCMS: 470.2 [M+H]⁺.

### Step 3: N-(4-(1-(4-Fluoro-3-hydroxyphenyl)-1H-indazol-5-yl)phenyl)propane-2-sulfonamide

Aqueous hydrochloric acid (3 M, 4.3 mL, 12.9 mmol) was added to a mixture of N-(4-(1-(4-fluoro-3-(methoxymethoxy)phenyl)-1H-indazol-5-yl)phenyl)propane-2-sulfonamide (300 mg, 0.64 mmol), THE (5 mL), and MeOH (5 mL) at room temperature. The mixture was heated at 50° C. for 2 h, poured into saturated NaHCO₃ (50 mL), and then extracted (3×50 mL EtOAc). The combined organic layers were washed (100 mL brine), dried (Na₂SO₄), filtered, and then concentrated. The crude product was purified by prep-HPLC [water (0.04% HCl)/MeCN] to give N-(4-(1-(4-fluoro-3-hydroxyphenyl)-1H-indazol-5-yl)phenyl)propane-2-sulfonamide (49 mg, 18%) as a pink solid. ¹H NMR (400 MHz, DMSO-d₆): δ 10.30 (s, 1H), 9.86 (s, 1H), 8.38 (s, 1H), 8.09 (s, 1H), 7.84 (d, 1H), 7.79 (d, 1H), 7.70 (d, 2H), 7.38-7.33 (m, 4H), 7.22-7.21 (m, 1H), 3.29-3.24 (m, 1H), 1.27 (d, 6H); LCMS: 426.1 [M+H]⁺.

The Compounds below were synthesized in a similar manner to that described for Compound 11.

| Cmpd | Structure | Name | [M + H]⁺ |
|---|---|---|---|
| 11.01 | 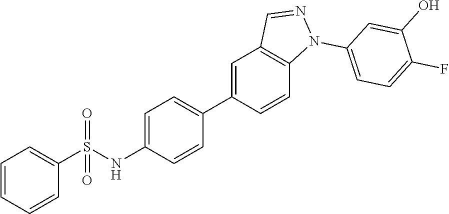 [SMILES: O=S(=O)(Nc1ccc(-c2ccc3c(cnn3-c3ccc(F)c(O)c3)c2)cc1)c1ccccc1] | N-(4-(1-(4-Fluoro-3-hydroxyphenyl)- 1H-indazol-5- yl)phenyl)benzenesulfonamide | 460.1 |
| 11.02 | 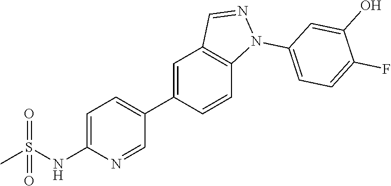 [SMILES: CS(=O)(=O)Nc1ccc(-c2ccc3c(cnn3-c3ccc(F)c(O)c3)c2)cn1] | N-(5-(1-(4-Fluoro-3-hydroxyphenyl)- 1H-indazol-5-yl)pyridine-2- yl)methanesulfonamide | 399.1 |

### Compound 12

### 3-Chloro-4-(1-(4-Fluoro-3-hydroxyphenyl)-1H-indazol-5-yl)benzoic acid

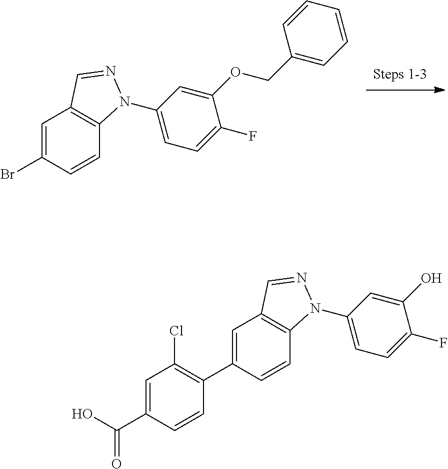 [SMILES: Fc1ccc(-n2ncc3cc(Br)ccc32)cc1OCc1ccccc1 ; O=C(O)c1ccc(-c2ccc3c(cnn3-c3ccc(F)c(O)c3)c2)c(Cl)c1]

### Step 1: Methyl 4-(1-(3-(benzyloxy)-4-fluorophenyl)-1H-indazol-5-yl)-3-chlorobenzoate

Methyl 4-(1-(3-(benzyloxy)-4-fluorophenyl)-1H-indazol-5-yl)-3-chlorobenzoate was synthesized from Intermediate 14.08 and (2-chloro-4-(methoxycarbonyl)phenyl)boronic acid in a similar manner to that described for Compound 4, Step 1. ¹H NMR (400 MHz, DMSO-d₆): δ 8.47 (d, J=0.6 Hz, 1H), 8.10 (d, J=1.7 Hz, 1H), 8.06-7.95 (m, 2H), 7.74-7.64 (m, 2H), 7.64-7.59 (m, 1H), 7.59-7.32 (m, 8H), 5.35 (s, 2H), 3.92 (s, 3H); LCMS 487.4 [M+H]⁺.

### Step 2: 4-(1-(3-(Benzyloxy)-4-fluorophenyl)-1H-indazol-5-yl)-3-chlorobenzoic acid

A mixture of methyl 4-(1-(3-(benzyloxy)-4-fluorophenyl)-1H-indazol-5-yl)-3-chlorobenzoate (550 mg, 1.13 mmol), methanol (5 mL), THF (10 mL), and NaOH (2 N, 3 mL, 6.0 mmol) was stirred for 4 h at room temperature and then concentrated. Water (50 mL), 1 N HCl (6 mL), and EtOAc (100 mL) were added. The mixture was stirred overnight. The solid was collected by filtration to give 4-(1-(3-(benzyloxy)-4-fluorophenyl)-1H-indazol-5-yl)-3-chlorobenzoic acid (225 mg) as a solid. ¹H NMR (400 MHz, DMSO-d₆): δ 13.39 (s, 1H), 8.46 (s, 1H), 8.07 (d, J=1.6 Hz, 1H), 8.02-7.98 (m, 2H), 7.71-7.59 (m, 3H), 7.58-7.30 (m, 8H), 5.36 (s, 2H); LCMS 473.1 [M+H]⁺.

### Step 3: 3-Chloro-4-(1-(4-fluoro-3-hydroxyphenyl)-1H-indazol-5-yl)benzoic acid

3-Chloro-4-(1-(4-fluoro-3-hydroxyphenyl)-1H-indazol-5-yl)benzoic acid was synthesized from 4-(1-(3-(benzyloxy)-4-fluorophenyl)-1H-indazol-5-yl)-3-chlorobenzoic acid in a similar manner to that described for Compound 2, Step 2. ¹H NMR (400 MHz, DMSO-d₆): δ 13.39 (s, 1H), 10.41 (s, 1H), 8.45 (s, 1H), 8.07 (d, J=1.6 Hz, 1H), 8.02-7.97 (m, 2H), 7.89 (d, J=8.8 Hz, 1H), 7.65 (d, J=7.9 Hz, 1H), 7.63-7.59 (m, 1H), 7.42-7.35 (m, 2H), 7.23 (td, J=3.3, 8.3 Hz, 1H); LCMS 382.9 [M+H]⁺.

### Compound 13

### 3-Chloro-4-(1-(4-fluoro-3-hydroxyphenyl)-1H-indazol-5-yl)benzamide

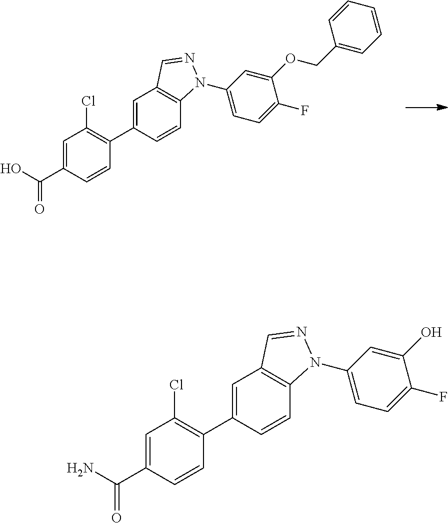 [SMILES: O=C(O)c1ccc(-c2ccc3c(cnn3-c3ccc(F)c(OCc4ccccc4)c3)c2)c(Cl)c1 ; NC(=O)c1ccc(-c2ccc3c(cnn3-c3ccc(F)c(O)c3)c2)c(Cl)c1]

HATU (80 mg, 0.22 mmol) was added to a mixture of Compound 12, Step 2 (80 mg, 0.17 mmol) and DIEA (0.20 mL, 1.15 mmol) in DMF (3 mL). The mixture was stirred for 10 min. Ammonium chloride (50 mg, 0.87 mmol) was added. The reaction was stirred for 40 min, diluted (20 mL EtOAc), washed (20 mL water and then 20 mL brine), dried (Na₂SO₄), and then concentrated. The residue was dissolved in THE (5.0 mL). 10% Pd/C (30 mg) was added. The mixture was stirred under hydrogen atmosphere for 100 min and filtered through a Celite pad. The filter cake was rinsed with 5 mL THF, and the filtrate was concentrated and purified by prep-HPLC to give 3-chloro-4-(1-(4-fluoro-3-hydroxyphenyl)-1H-indazol-5-yl)benzamide (35 mg, 53%) as a white solid. ¹H NMR (400 MHz, DMSO-d₆): δ 10.40 (s, 1H), 8.44 (s, 1H), 8.17 (s, 1H), 8.09 (d, J=1.7 Hz, 1H), 7.99 (d, J=0.7 Hz, 1H), 7.94 (dd, J=1.7, 7.9 Hz, 1H), 7.88 (d, J=8.8 Hz, 1H), 7.60 (d, J=7.8 Hz, 3H), 7.41-7.32 (m, 2H), 7.23 (td, J=3.4, 8.3 Hz, 1H); LCMS: 381.9 [M+H]⁺.

### Compound 14

### 5-(3-Chloro-4-hydroxyphenyl)-1-(4-fluoro-3-hydroxyphenyl)-N,N-dimethyl-1H-indazole-3-carboxamide

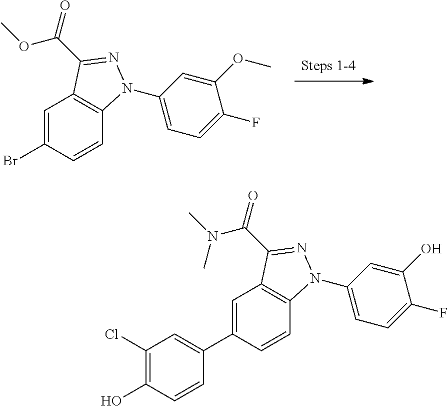

### Step 1: Methyl 5-(3-chloro-4-methoxyphenyl)-1-(4-fluoro-3-methoxyphenyl)-1H-indazole-3-carboxylate

Pd(PPh₃)₄ (76 mg, 0.066 mmol) was added to a mixture of Intermediate 14.09 (500 mg, 1.32 mmol), (3-chloro-4-methoxyphenyl)boronic acid (295 mg, 1.58 mmol), and Cs₂CO₃ (859 mg, 2.64 mmol) in DME (6 mL) and H₂O (3 mL) under N₂. The mixture was stirred at 80° C. overnight, allowed to cool to room temperature, poured into water (20 mL), and then extracted (3×15 mL EtOAc). The combined organic layers were washed (10 mL brine), dried (Na₂SO₄), filtered, and then concentrated. The residue was purified by silica gel chromatography (10% EtOAc/petroleum ether) to give methyl 5-(3-chloro-4-methoxyphenyl)-1-(4-fluoro-3-methoxyphenyl)-1H-indazole-3-carboxylate (550 mg, 85%, ˜90% purity) as a white solid. ¹H NMR (400 MHz, DMSO-d₆): δ 8.33 (s, 1H), 7.96-7.84 (m, 2H), 7.82-7.77 (m, 1H), 7.72-7.68 (m, 1H), 7.59-7.46 (m, 2H), 7.43-7.34 (m, 1H), 7.33-7.23 (m, 1H), 4.02-3.90 (m, 9H); LCMS: 441.1 [M+H]⁺.

### Step 2: 5-(3-Chloro-4-methoxyphenyl)-1-(4-fluoro-3-methoxyphenyl)-1H-indazole-3-carboxylic acid

A mixture of methyl 5-(3-chloro-4-methoxyphenyl)-1-(4-fluoro-3-methoxyphenyl)-1H-indazole-3-carboxylate (550 mg, 1.12 mmol), LiOH·H₂O (131 mg, 3.12 mmol), THF (10 mL), MeOH (5 mL), and H₂O (5 mL) was stirred at room temperature overnight and then concentrated to remove the organic solvents. The pH was adjusted with 1 N HCl to pH=2, and the suspension was filtered. The filter cake was washed with ice-cold water (5 mL) and dried under vacuum to give 5-(3-chloro-4-methoxyphenyl)-1-(4-fluoro-3-methoxyphenyl)-1H-indazole-3-carboxylic acid (320 mg) as yellow solid. ¹H NMR (400 MHz, DMSO-d₆): δ 13.40 (s, 1H), 8.34 (s, 1H), 7.97-7.83 (m, 2H), 7.78 (d, 1H), 7.69 (d, 1H), 7.59-7.46 (m, 2H), 7.43-7.35 (m, 1H), 7.29 (d, 1H), 3.97 (s, 3H), 3.92 (s, 3H); LCMS: 427.1 [M+H]⁺.

### Step 3: 5-(3-Chloro-4-methoxyphenyl)-1-(4-fluoro-3-methoxyphenyl)-N,N-dimethyl-1H-indazole-3-carboxamide

A mixture of 5-(3-chloro-4-methoxyphenyl)-1-(4-fluoro-3-methoxyphenyl)-1H-indazole-3-carboxylic acid (140 mg, 0.328 mmol), dimethylamine hydrochloride (40 mg, 0.49 mmol), HATU (150 mg, 0.39 mmol), DIPEA (127 mg, 0.98 mmol), and DCM (2 mL) was stirred at room temperature for 3 h, poured into water (10 mL), and then extracted (3×10 mL DCM). The combined organic layers were washed (10 mL brine), dried (Na₂SO₄), filtered, and then concentrated. The residue was purified by silica gel chromatography (60% EtOAc/petroleum ether) to give 5-(3-chloro-4-methoxyphenyl)-1-(4-fluoro-3-methoxyphenyl)-N,N-dimethyl-1H-indazole-3-carboxamide (75 mg, 50%) as a white solid. ¹H NMR (400 MHz, CDCl₃): δ 8.38 (s, 1H), 7.76-7.64 (m, 3H), 7.55 (dd, 1H), 7.34 (d, 1H), 7.30-7.24 (m, 2H), 7.02 (d, 1H), 3.98 (d, 6H), 3.49 (s, 3H), 3.26 (s, 3H); LCMS: 454.2 [M+H]⁺.

### Step 4: 5-(3-Chloro-4-hydroxyphenyl)-1-(4-fluoro-3-hydroxyphenyl)-N,N-dimethyl-1H-indazole-3-carboxamide

Boron tribromide (111 μL 1.16 mmol) was added to a mixture of 5-(3-chloro-4-methoxy-phenyl)-1-(4-fluoro-3-methoxy-phenyl)-N,N-dimethyl-indazole-3-carboxamide (75 mg, 0.17 mmol) and DCM (5 mL) at −78° C. The mixture was stirred at room temperature for 2 h, quenched by slow addition of MeOH (10 mL), poured into saturated NaHCO₃ (20 mL), and then extracted (3×20 mL EtOAc). The combined organic layers were washed (20 mL brine), dried (Na₂SO₄), filtered, and then concentrated. The residue was purified by prep-HPLC [water (0.04% HCl)/MeCN] to give 5-(3-chloro-4-hydroxy-phenyl)-1-(4-fluoro-3-hydroxy-phenyl)-N,N-dimethyl-indazole-3-carboxamide (24 mg, 34%) as a white solid. ¹H NMR (400 MHz, DMSO-d₆): δ 10.45 (s, 1H), 10.34 (s, 1H), 8.18 (s, 1H), 7.83-7.82 (m, 2H), 7.67 (s, 1H), 7.53-7.51 (m, 1H), 7.40-7.39 (m, 2H), 7.37-7.36 (m, 1H), 7.11-7.09 (m, 1H), 3.37 (s, 3H), 3.12 (s, 3H); LCMS: 426.1 [M+H]⁺.

### Compound 15

### 3-(4-(1-(4-Fluoro-3-hydroxyphenyl)-1H-indazol-5-yl)phenoxy)propanoic acid

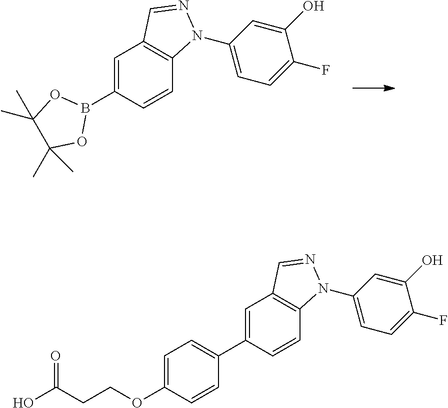 [SMILES: CC1(C)OB(c2ccc3c(cnn3-c3ccc(F)c(O)c3)c2)OC1(C)C ; O=C(O)CCOc1ccc(-c2ccc3c(cnn3-c3ccc(F)c(O)c3)c2)cc1]

Pd₂(dba)₃ (103 mg, 0.113 mmol) was added to a mixture of 3-(4-bromophenoxy)propanoic acid (332 mg, 1.36 mmol), Intermediate 19.01 (400 mg), Xphos (108 mg, 0.23 mmol), Cs₂CO₃ (1.10 g, 3.39 mmol), water (3 mL), and dioxane (9 mL)under N₂. The reaction mixture was stirred at 100° C. overnight, allowed to cool to room temperature, and then poured into water (20 mL). The pH was adjusted with 1 N HCl to pH˜5, and the mixture was extracted (3×20 mL EtOAc). The combined organic layers were dried (Na₂SO₄), filtered, and then concentrated. The residue was purified by silica gel chromatography (80% EtOAc/petroleum ether), and then stirred in a mixture of DCM (2 mL) and n-hexane (5 mL) at room temperature overnight. The mixture was filtered and the filter cake was washed with ice-cold n-hexane (2 mL) to give 3-(4-(1-(4-fluoro-3-hydroxyphenyl)-1H-indazol-5-yl)phenoxy)propanoic acid (67 mg, 14%) as a light pink solid. ¹H NMR (400 MHz, DMSO-d₆): δ 12.39 (s, 1H), 10.38 (s, 1H), 8.37 (s, 1H), 8.08 (s, 1H), 7.91-7.81 (m, 1H), 7.80-7.73 (m, 1H), 7.66 (d, 2H), 7.44-7.30 (m, 2H), 7.25-7.15 (m, 1H), 7.05 (d, 2H), 4.22 (t, 2H), 2.72 (t, 2H); LCMS: 391.1 [M−H]⁻.

### Compound 16

### 6-(1-(3,4-Difluoro-5-hydroxyphenyl)-1H-indazol-5-yl)pyridine-3-ol

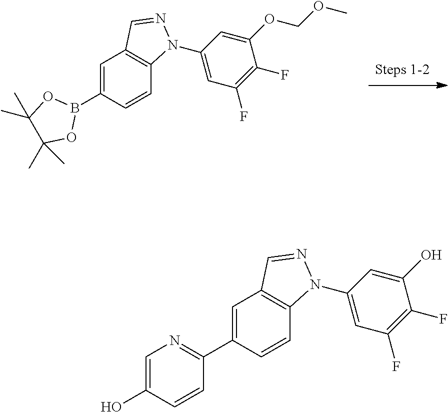

### Step 1: 1-(3,4-Difluoro-5-(methoxymethoxy)phenyl)-5-(5-(methoxymethoxy)pyridine-2-yl)-1H-indazole

Aqueous Na₂CO₃ (2 M, 0.72 mL, 1.44 mmol) and Pd(PPh₃)₄ (28 mg, 0.02 mol) were added to a solution of Intermediate 19 (200 mg, 0.48 mmol) and 2-bromo-5-(methoxymethoxy)pyridine (158 mg, 0.72 mmol) in EtOH (1 mL) and toluene (4 mL) under N₂. The mixture was degassed with 3 vacuum/N₂ cycles, stirred at 80° C. for 16 h, cooled to room temperature, poured into H₂O (10 mL), and then extracted (3×20 mL EtOAc). The combined organic layers were washed (2×10 mL brine), dried (Na₂SO₄), filtered, and then concentrated. The residue was purified by silica gel chromatography (10-20% EtOAc/petroleum ether) to give 1-(3,4-difluoro-5-(methoxymethoxy)phenyl)-5-(5-(methoxymethoxy)pyridine-2-yl)-1H-indazole (160 mg, 62%) as a white solid. ¹H NMR (400 MHz, DMSO-d₆): δ 8.52 (s, 1H), 8.48 (s, 1H), 8.45 (d, 1H), 8.22-8.24 (m, 1H), 8.00-8.02 (m, 1H), 7.94-7.96 (m, 1H), 7.53-7.59 (m, 3H), 5.43 (s, 2H), 5.31 (s, 2H), 3.47 (s, 3H), 3.43 (s, 3H); LCMS: 428.2 [M+H]⁺.

### Step 2: 6-(1-(3,4-Difluoro-5-hydroxyphenyl)-1H-indazol-5-yl)pyridine-3-ol

Aqueous HCl (3 M, 1.40 mL, 4.2 mmol) was added to a solution of 1-(3,4-difluoro-5-(methoxymethoxy)phenyl)-5-(5-(methoxymethoxy)pyridine-2-yl)-1H-indazole (140 mg, 0.33 mmol) in MeOH (0.5 mL) and THE (0.5 mL). The mixture was stirred at 90° C. for 0.5 h and then cooled to room temperature. Aqueous saturated NaHCO₃ was added to adjust the pH to ˜8, and the mixture was extracted (3×40 mL EtOAc). The combined organic phases were concentrated and purified by silica gel chromatography (10-50% EtOAc/petroleum ether). The crude product was purified further by prep-HPLC [water (0.04% NH₃·H₂O+10 mM NH₄HCO₃)-MeCN] to give 6-(1-(3,4-difluoro-5-hydroxyphenyl)-1H-indazol-5-yl)pyridine-3-ol (29 mg, 26%) as a yellow solid. ¹H NMR (400 MHz, DMSO-d₆): δ 10.93 (s, 1H), 10.01 (s, 1H), 8.43-8.44 (m, 2H), 8.18-8.24 (m, 2H), 7.87-7.91 (m, 2H), 7.24-7.31 (m, 3H); LCMS: 340.1 [M+H]⁺.

The Compounds below were synthesized in a similar manner to that described for Compound 16.

| Cmpd | Structure | Name | [M + H]⁺ |
|---|---|---|---|
| 16.01 | 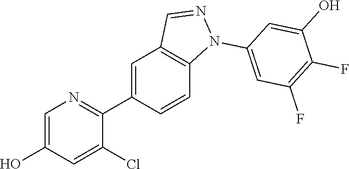 [SMILES: Oc1cnc(-c2ccc3c(cnn3-c3cc(O)c(F)c(F)c3)c2)c(Cl)c1] | 5-Chloro-6-(1-(3,4-difluoro-5- hydroxyphenyl)-1H-indazol-5- yl)pyridine-3-ol | 373.9 |
| 16.02 | 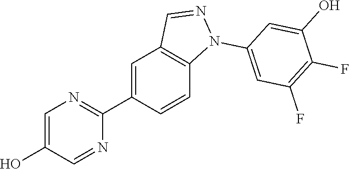 [SMILES: Oc1cnc(-c2ccc3c(cnn3-c3cc(O)c(F)c(F)c3)c2)nc1] | 2-(1-(3,4-Difluoro-5- hydroxyphenyl)-1H-indazol-5- yl)pyrimidin-5-ol | 341.0 |
| 16.03 | 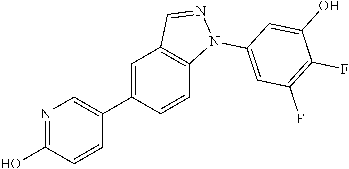 [SMILES: Oc1ccc(-c2ccc3c(cnn3-c3cc(O)c(F)c(F)c3)c2)cn1] | 5-(1-(3,4-Difluoro-5- hydroxyphenyl)-1H-indazol-5- yl)pyridine-2-ol | 339.9 |

### Compound 17

### 5-(5-(3-Chloro-4-hydroxyphenyl)-1H-indazol-1-yl)pyridin-3-ol

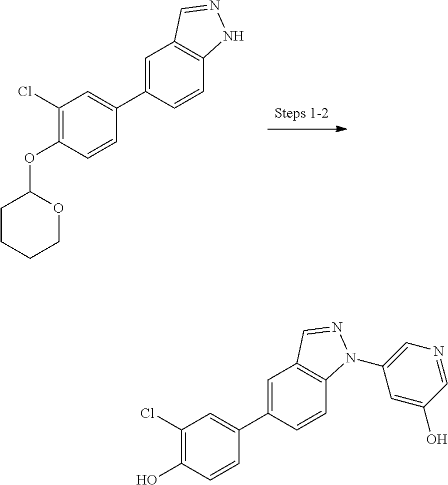 [SMILES: Clc1cc(-c2ccc3[nH]ncc3c2)ccc1OC1CCCCO1 ; Oc1cncc(-n2ncc3cc(-c4ccc(O)c(Cl)c4)ccc32)c1]

### Step 1: 5-(5-(3-Chloro-4-((tetrahydro-2H-pyran-2-yl)oxy)phenyl)-1H-indazol-3-yl)pyridin-3-ol

A mixture of Intermediate 12 (101 mg, 0.31 mmol), 5-iodopyridin-3-ol (103 mg, 0.47 mmol), CuI (11 mg, 0.06 mmol), K₂CO₃ (89 mg, 0.64 mmol), L-proline (14 mg, 0.12 mmol), and DMSO (2 mL) was degassed with 3 vacuum/N₂ cycles, stirred at 100° C. overnight, stirred at 110° C. for an additional 2 h, allowed to cool to room temperature, and then diluted (20 mL EtOAc and 20 mL water). The organic layer was washed (20 mL brine), dried (Na₂SO₄), filtered, concentrated, and then purified by silica gel chromatography (20-80% EtOAc/hexanes) to give 5-(5-(3-chloro-4-((tetrahydro-2H-pyran-2-yl)oxy)phenyl)-1H-indazol-3-yl)pyridin-3-ol (35 mg, 27%) as a tan solid. ¹H NMR (400 MHz, DMSO-d₆): δ 10.44 (s, 1H), 8.56 (br s, 1H), 8.47 (s, 1H), 8.23-8.14 (m, 2H), 7.96-7.91 (m, 1H), 7.86-7.80 (m, 2H), 7.67 (dd, J=2.3, 8.7 Hz, 1H), 7.59 (t, J=2.2 Hz, 1H), 7.37 (d, J=8.7 Hz, 1H), 5.70 (t, J=2.8 Hz, 1H), 3.82-3.73 (m, 1H), 3.63-3.56 (m, 1H), 1.98-1.91 (m, 1H), 1.88-1.82 (m, 2H), 1.71-1.46 (m, 3H); LCMS: 422.0 [M+H]⁺.

### Step 2: 5-(5-(3-Chloro-4-hydroxyphenyl)-1H-indazol-1-yl)pyridin-3-ol

Aqueous hydrochloric acid (0.20 mL, 0.20 mmol) was added to a solution of 5-(5-(3-chloro-4-((tetrahydro-2H-pyran-2-yl)oxy)phenyl)-1H-indazol-3-yl)pyridin-3-ol (33 mg, 0.08 mmol) in methanol (2 mL) and THF (1 mL) at room temperature. The reaction was stirred for 75 min and then diluted (20 mL EtOAc and 20 mL water). The organic layer was washed (2×20 mL saturated NaHCO₃ and then 20 mL brine), dried (Na₂SO₄), filtered, concentrated, and then purified by prep-HPLC (16-26% CH₃CN in H₂O with 0.1% TFA) to give 5-(5-(3-chloro-4-hydroxyphenyl)-1H-indazol-1-yl)pyridin-3-ol (20 mg, 75%) as a tan solid. ¹H NMR (400 MHz, DMSO-d₆): δ 10.44 (s, 1H), 10.32 (s, 1H), 8.55 (d, J=1.8 Hz, 1H), 8.45 (s, 1H), 8.18 (d, J=2.2 Hz, 1H), 8.14-8.10 (m, 1H), 7.92 (d, J=8.9 Hz, 1H), 7.79 (dd, J=1.7, 8.9 Hz, 1H), 7.71 (d, J=2.3 Hz, 1H), 7.59 (t, J=2.3 Hz, 1H), 7.57-7.52 (m, 1H), 7.08 (d, J=8.6 Hz, 1H); LCMS: 337.9 [M+H]⁺.

### Compound 18

### 5-(5-(4,4-Dimethylcyclohex-1-en-1-yl)-6-fluoro-1H-indazol-1-yl)-2-fluoro-3-(trifluoromethyl)phenol

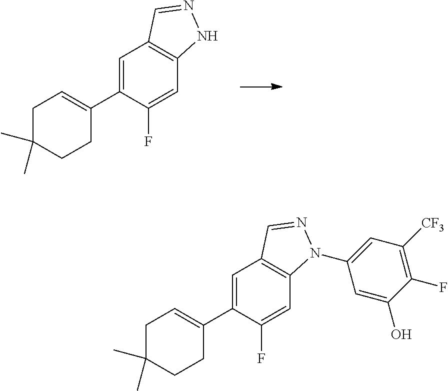 [SMILES: CC1(C)CC=C(c2cc3cn[nH]c3cc2F)CC1 ; CC1(C)CC=C(c2cc3cnn(-c4cc(O)c(F)c(C(F)(F)F)c4)c3cc2F)CC1]

A mixture of Intermediate 11.03 (20 mg, 0.082 mmol), 5-bromo-2-fluoro-3-(trifluoromethyl)phenol (25 mg, 0.10 mmol), K₃PO₄ (43 mg, 0.20 mmol), CuI (3.1 mg, 0.016 mmol), and trans-N,N′-dimethylcyclohexane-1,2-diamine (0.010 mL, 0.065 mmol) in toluene (1.0 mL) was heated at 100° C. overnight. The reaction mixture was diluted with EtOAc and washed with water. The aqueous layer was extracted with EtOAc. The combined organics were washed with brine, dried (MgSO₄), and then concentrated. The residue was purified by prep-HPLC to give 5-(5-(4,4-dimethylcyclohex-1-en-1-yl)-6-fluoro-1H-indazol-1-yl)-2-fluoro-3-(trifluoromethyl)phenol (1.0 mg, 2.6%) as a beige solid. ¹H NMR (400 MHz, MeOD-d₄): δ 8.23 (s, 1H), 7.72 (d, J=7.2 Hz, 1H), 7.54 (dd, J=2.5, 7.2 Hz, 1H), 7.45-7.39 (m, 2H), 5.86 (br s, 1H), 2.44 (br s, 2H), 2.03 (br d, J=3.5 Hz, 2H), 1.56 (t, J=6.4 Hz, 2H), 1.03 (s, 6H); LCMS 423.2 [M+H]⁺.

The Compounds below were synthesized in a similar manner to that described for Compound 18.

| Cmpd | Structure | Name | [M + H]⁺ |
|---|---|---|---|
| 18.01 | 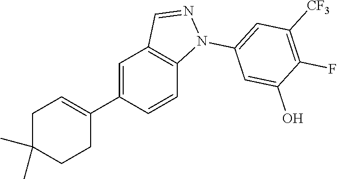 [SMILES: CC1(C)CC=C(c2ccc3c(cnn3-c3cc(O)c(F)c(C(F)(F)F)c3)c2)CC1] | 5-(5-(4,4-Dimethylcyclohex-1-en-1- yl)-1H-indazol-1-yl)-2-fluoro-3- (trifluoromethyl)phenol | 404.9 |
| 18.02 | 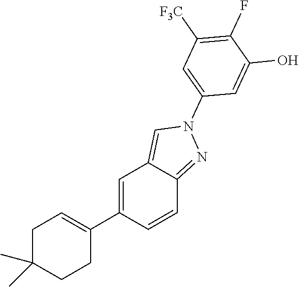 [SMILES: CC1(C)CC=C(c2ccc3nn(-c4cc(O)c(F)c(C(F)(F)F)c4)cc3c2)CC1] | 5-(5-(4,4-Dimethylcyclohex-1-en-1- yl)-2H-indazol-2-yl)-2-fluoro-3- (trifluoromethyl)phenol | 405.2 |

### Compound 19

### 5-(6-Chloro-5-(4-(methylsulfonyl)piperazin-1-yl)-1H-indazol-1-yl)-2-fluoro-3-(trifluoromethyl)phenol

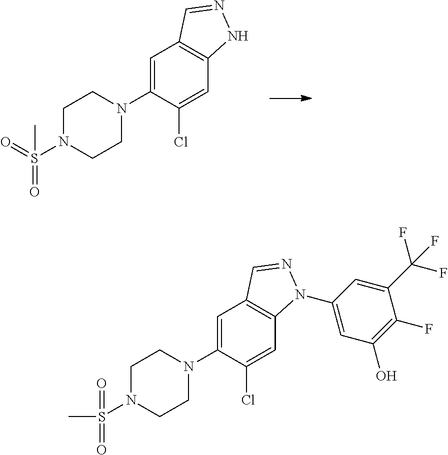 [SMILES: CS(=O)(=O)N1CCN(c2cc3cn[nH]c3cc2Cl)CC1 ; CS(=O)(=O)N1CCN(c2cc3cnn(-c4cc(O)c(F)c(C(F)(F)F)c4)c3cc2Cl)CC1]

A mixture of Intermediate 13 (195 mg, 0.62 mmol), 5-bromo-2-fluoro-3-(trifluoromethyl)phenol (193 mg, 0.74 mmol), K₃PO₄ (329 mg, 1.55 mmol), CuI (24 mg, 0.12 mmol), and trans-N,N-dimethylcyclohexane-1,2-diamine (0.08 mL, 0.50 mmol) in toluene (4 mL) was heated at 100° C. overnight, allowed to cool to rt, diluted with water, and then extracted with EtOAc. The aqueous layer was extracted with EtOAc. The combined organics were washed with brine, dried (MgSO₄), concentrated, and then purified by prep-HPLC (20-60 mp CH₃CN in water with 0.1% TFA). The fractions were combined, diluted with EtOAc, and then washed with saturated NaHCO₃ aqueous solution and then brine. The organics were dried (MgSO₄) and concentrated to give 5-(6-chloro-5-(4-(methylsulfonyl)piperazin-1-yl)-1H-indazol-1-yl)-2-fluoro-3-(trifluoromethyl)phenol (60 mg, 190) as a beige solid. ¹H NMR (400 MHz, DMSO-d₆): δ 11.15 (s, 1H), 8.37 (s, 1H), 7.97 (s, 1H), 7.71 (s, 1H), 7.64 (dd, J=2.4, 7.2 Hz, 1H), 7.45 (dd, J=2.5, 5.0 Hz, 1H), 3.37-3.29 (m, 4H), 3.16-3.06 (m, 4H), 2.98 (s, 3H); LCMS 492.9 [M+H]⁺.

The Compounds below were synthesized in a similar manner to that described for Compound 19.

| Cmpd | Structure | Name | [M + H]⁺ |
|---|---|---|---|
| 19.01 | 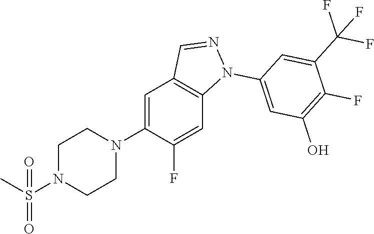 [SMILES: CS(=O)(=O)N1CCN(c2cc3cnn(-c4cc(O)c(F)c(C(F)(F)F)c4)c3cc2F)CC1] | 2-Fluoro-5-(6-fluoro-5-(4- (methylsulfonyl)piperazin-1-yl)-1H- indazol-1-yl)-3- (trifluoromethyl)phenol | 476.9 |
| 19.02 | 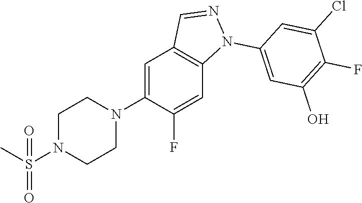 [SMILES: CS(=O)(=O)N1CCN(c2cc3cnn(-c4cc(O)c(F)c(Cl)c4)c3cc2F)CC1] | 3-Chloro-2-fluoro-5-(6-fluoro-5-(4- (methylsulfonyl)piperazin-1-yl)-1H- indazol-1-yl)phenol | 443.0 |
| 19.03 | 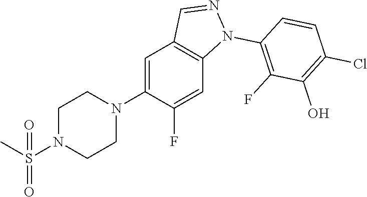 [SMILES: CS(=O)(=O)N1CCN(c2cc3cnn(-c4ccc(Cl)c(O)c4F)c3cc2F)CC1] | 6-Chloro-2-fluoro-3-(6-fluoro-5-(4- (methylsulfonyl)piperazin-1-yl)-1H- indazol-1-yl)phenol | 443.0 |
| 19.04 | 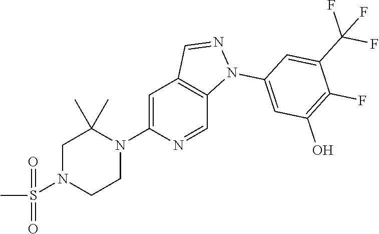 [SMILES: CC1(C)CN(S(C)(=O)=O)CCN1c1cc2cnn(-c3cc(O)c(F)c(C(F)(F)F)c3)c2cn1] | 5-(5-(2,2-Dimethyl-4- (methylsulfonyl)piperazin-1-yl)-1H- pyrazolo[3,4-c]pyridine-1-yl)-2- fluoro-3-(trifluoromethyl)phenol | 488.0 |
| 19.05 (2) | 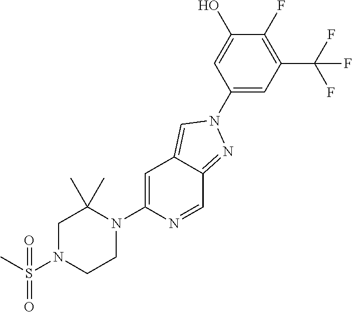 [SMILES: CC1(C)CN(S(C)(=O)=O)CCN1c1cc2cn(-c3cc(O)c(F)c(C(F)(F)F)c3)nc2cn1] | 5-(5-(2,2-Dimethyl-4- (methylsulfonyl)piperazin-1-yl)-2H- pyrazolo[3,4-c]pyridine-2-yl)-2- fluoro-3-(trifluoromethyl)phenol | 488.0 |
| 19.06 | 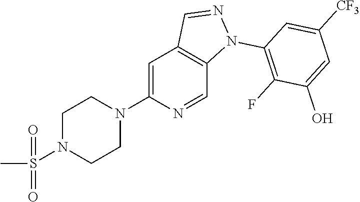 [SMILES: CS(=O)(=O)N1CCN(c2cc3cnn(-c4cc(C(F)(F)F)cc(O)c4F)c3cn2)CC1] | 2-Fluoro-3-(5-(4- (methylsulfonyl)piperazin-1-yl)-1H- pyrazolo[3,4-c]pyridine-1-yl)-5- (trifluoromethyl)phenol | 460.1 |
| 19.07 (1) | 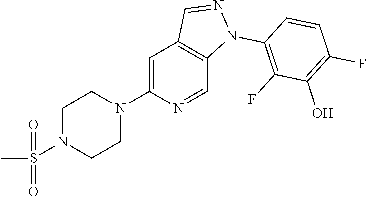 [SMILES: CS(=O)(=O)N1CCN(c2cc3cnn(-c4ccc(F)c(O)c4F)c3cn2)CC1] | 2,6-Difluoro-3-(5-(4- (methylsulfonyl)piperazin-1-yl)-1H- pyrazolo[3,4-c]pyridine-1-yl)phenol | 410.0 |
| 19.08 (1,2) | 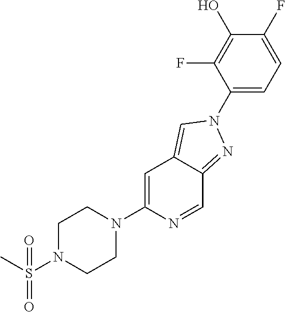 [SMILES: CS(=O)(=O)N1CCN(c2cc3cn(-c4ccc(F)c(O)c4F)nc3cn2)CC1] | 2,6-Difluoro-3-(5-(4- (methylsulfonyl)piperazin-1-yl)-2H- pyrazolo[3,4-c]pyridine-2-yl)phenol | 409.9 |
| 19.09 (1) | 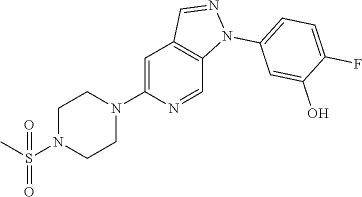 [SMILES: CS(=O)(=O)N1CCN(c2cc3cnn(-c4ccc(F)c(O)c4)c3cn2)CC1] | 2-Fluoro-5-(5-(4- (methylsulfonyl)piperazin-1-yl)-1H- pyrazolo[3,4-c]pyridine-1-yl)phenol | 392.0 |
| 19.10 (1,2) | 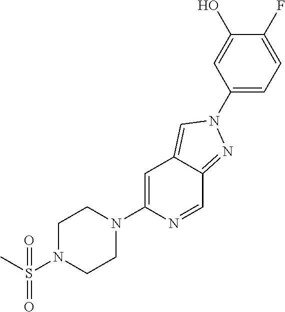 [SMILES: CS(=O)(=O)N1CCN(c2cc3cn(-c4ccc(F)c(O)c4)nc3cn2)CC1] | 2-Fluoro-5-(5-(4- (methylsulfonyl)piperazin-1-yl)-2H- pyrazolo[3,4-c]pyridine-2-yl)phenol | 392.0 |
| 19.11 (1) | 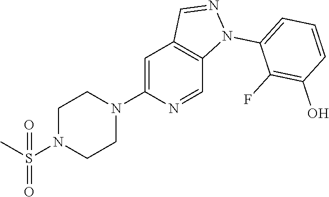 [SMILES: CS(=O)(=O)N1CCN(c2cc3cnn(-c4cccc(O)c4F)c3cn2)CC1] | 2-Fluoro-3-(5-(4- (methylsulfonyl)piperazin-1-yl)-1H- pyrazolo[3,4-c]pyridine-1-yl)phenol | 392.0 |
| 19.12 (1) | 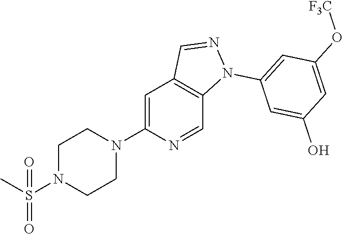 [SMILES: CS(=O)(=O)N1CCN(c2cc3cnn(-c4cc(O)cc(OC(F)(F)F)c4)c3cn2)CC1] | 3-(5-(4-(Methylsulfonyl)piperazin-1- yl)-1H-pyrazolo[3,4-c]pyridine-1-yl)- 5-(trifluoromethoxy)phenol | 458.2 |
| 19.13 (1,2) | 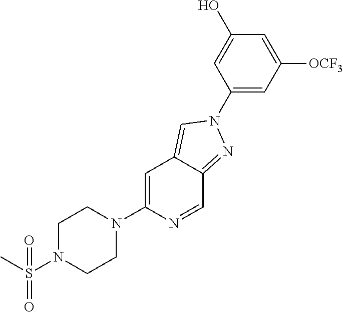 [SMILES: CS(=O)(=O)N1CCN(c2cc3cn(-c4cc(O)cc(OC(F)(F)F)c4)nc3cn2)CC1] | 3-(5-(4-(Methylsulfonyl)piperazin-1- yl)-2H-pyrazolo[3,4-c]pyridin-2-yl)- 5-(trifluoromethoxy)phenol | 458.0 |
| 19.14 (1) | 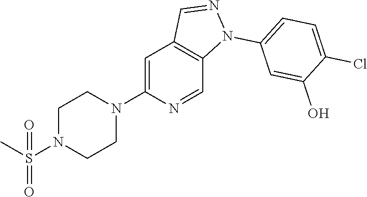 [SMILES: CS(=O)(=O)N1CCN(c2cc3cnn(-c4ccc(Cl)c(O)c4)c3cn2)CC1] | 2-Chloro-5-(5-(4- (methylsulfonyl)piperazin-1-yl)-1H- pyrazolo[3,4-c]pyridine-1-yl)phenol | 408.0 |
| 19.15 (1,2) | 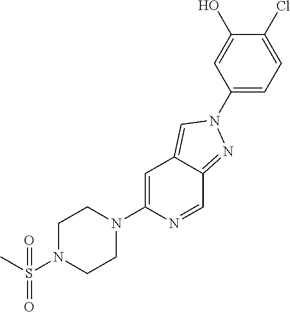 [SMILES: CS(=O)(=O)N1CCN(c2cc3cn(-c4ccc(Cl)c(O)c4)nc3cn2)CC1] | 2-Chloro-5-(5-(4- (methylsulfonyl)piperazin-1-yl)-2H- pyrazolo[3,4-c]pyridine-2-yl)phenol | 408.0 |
| 19.16 (1) | 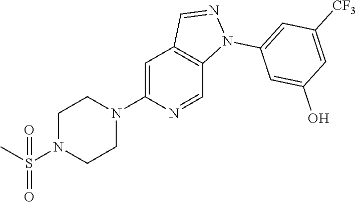 [SMILES: CS(=O)(=O)N1CCN(c2cc3cnn(-c4cc(O)cc(C(F)(F)F)c4)c3cn2)CC1] | 3-(5-(4-(Methylsulfonyl)piperazin-1- yl)-1H-pyrazolo[3,4-c]pyridine-1-yl)- 5-(trifluoromethyl)phenol | 442.0 |
| 19.17 (1) | 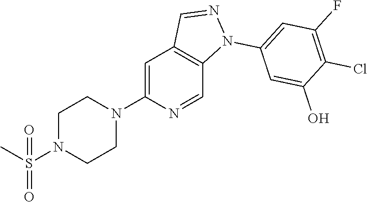 [SMILES: CS(=O)(=O)N1CCN(c2cc3cnn(-c4cc(O)c(Cl)c(F)c4)c3cn2)CC1] | 2-Chloro-3-fluoro-5-(5-(4- (methylsulfonyl)piperazin-1-yl)-1H- pyrazolo[3,4-c]pyridine-1-yl)phenol | 426.0 |
| 19.18 (1,2) | 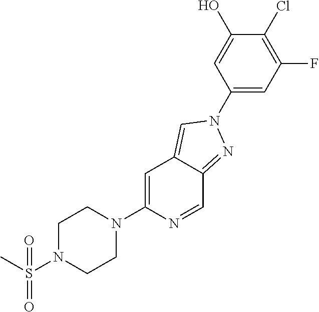 [SMILES: CS(=O)(=O)N1CCN(c2cc3cn(-c4cc(O)c(Cl)c(F)c4)nc3cn2)CC1] | 2-Chloro-3-fluoro-5-(5-(4- (methylsulfonyl)piperazin-1-yl)-2H- pyrazolo[3,4-c]pyridine-2-yl)phenol | 425.9 |
| 19.19 (1) | 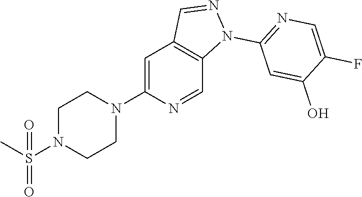 [SMILES: CS(=O)(=O)N1CCN(c2cc3cnn(-c4cc(O)c(F)cn4)c3cn2)CC1] | 5-Fluoro-2-(5-(4- (methylsulfonyl)piperazin-1-yl)-1H- pyrazolo[3,4-c]pyridine-1- yl)pyridine-4-ol | 393.0 |
| 19.20 (1,2) | 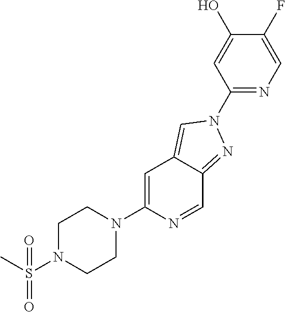 [SMILES: CS(=O)(=O)N1CCN(c2cc3cn(-c4cc(O)c(F)cn4)nc3cn2)CC1] | 5-Fluoro-2-(5-(4- (methylsulfonyl)piperazin-1-yl)-2H- pyrazolo[3,4-c]pyridine-2- yl)pyridine-4-ol | 393.0 |
| 19.21 (1) | 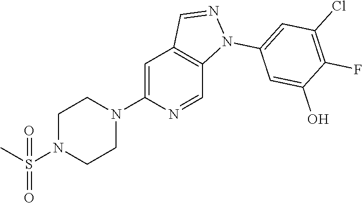 [SMILES: CS(=O)(=O)N1CCN(c2cc3cnn(-c4cc(O)c(F)c(Cl)c4)c3cn2)CC1] | 3-Chloro-2-fluoro-5-(5-(4- (methylsulfonyl)piperazin-1-yl)-1H- pyrazolo[3,4-c]pyridine-1-yl)phenol | 426.0 |
| 19.22 (1,2) | 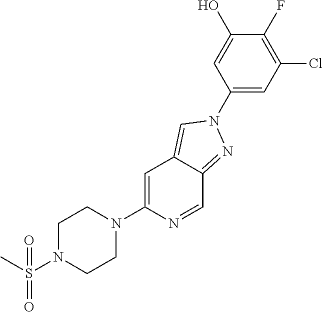 [SMILES: CS(=O)(=O)N1CCN(c2cc3cn(-c4cc(O)c(F)c(Cl)c4)nc3cn2)CC1] | 3-Chloro-2-fluoro-5-(5-(4- (methylsulfonyl)piperazin-1-yl)-2H- pyrazolo[3,4-c]pyridine-2-yl)phenol | 426.0 |
| 19.23 (1) | 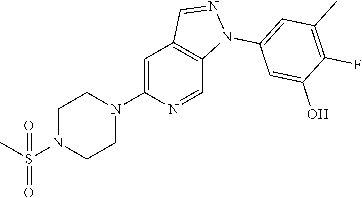 [SMILES: Cc1cc(-n2ncc3cc(N4CCN(S(C)(=O)=O)CC4)ncc32)cc(O)c1F] | 2-Fluoro-3-methyl-5-(5-(4- (methylsulfonyl)piperazin-1-yl)-1H- pyrazolo[3,4-c]pyridine-1-yl)phenol | 406.1 |
| 19.24 (1,2) | 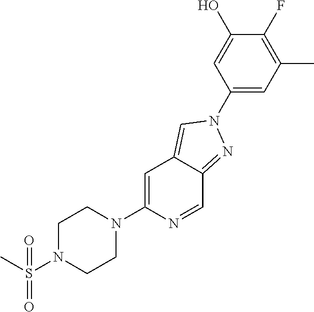 | 2-Fluoro-3-methyl-5-(5-(4- (methylsulfonyl)piperazin-1-yl)-2H- pyrazolo[3,4-c]pyridine-2-yl)phenol | 406.0 |
| 19.25 (1) | 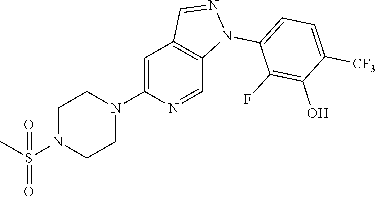 [SMILES: CS(=O)(=O)N1CCN(c2cc3cnn(-c4ccc(C(F)(F)F)c(O)c4F)c3cn2)CC1] | 2-Fluoro-3-(5-(4- (methylsulfonyl)piperazin-1-yl)-1H- pyrazolo[3,4-c]pyridine-1-yl)-6- (trifluoromethyl)phenol | 460.1 |
| 19.26 (1,2) | 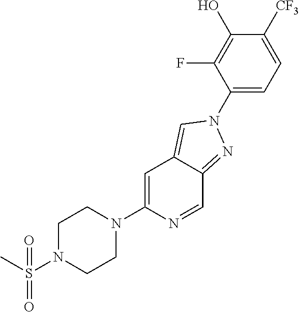 [SMILES: CS(=O)(=O)N1CCN(c2cc3cn(-c4ccc(C(F)(F)F)c(O)c4F)nc3cn2)CC1] | 2-Fluoro-3-(5-(4- (methylsulfonyl)piperazin-1-yl)-2H- pyrazolo[3,4-c]pyridine-2-yl)-6- (trifluoromethyl)phenol | 460.1 |
| 19.27 (1) | 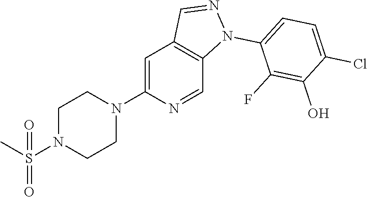 [SMILES: CS(=O)(=O)N1CCN(c2cc3cnn(-c4ccc(Cl)c(O)c4F)c3cn2)CC1] | 6-Chloro-2-fluoro-3-(5-(4- (methylsulfonyl)piperazin-1-yl)-1H- pyrazolo[3,4-c]pyridine-1-yl)phenol | 426.0 |
| 19.28 (1) | 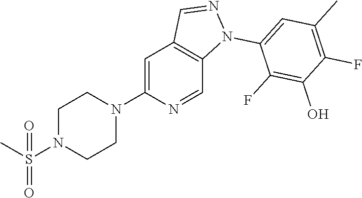 [SMILES: Cc1cc(-n2ncc3cc(N4CCN(S(C)(=O)=O)CC4)ncc32)c(F)c(O)c1F] | 2,6-Difluoro-3-methyl-5-(5-(4- (methylsulfonyl)piperazin-1-yl)-1H- pyrazolo[3,4-c]pyridine-1-yl)phenol | 424.3 |
| 19.29 (1) | 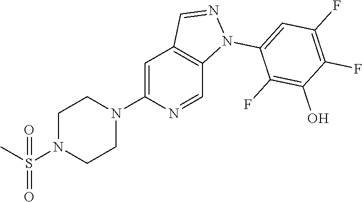 [SMILES: CS(=O)(=O)N1CCN(c2cc3cnn(-c4cc(F)c(F)c(O)c4F)c3cn2)CC1] | 2,3,6-Trifluoro-5-(5-(4- (methylsulfonyl)piperazin-1-yl)-1H- pyrazolo[3,4-c]pyridine-1-yl)phenol | 428.1 |
| 19.30 (1,2) | 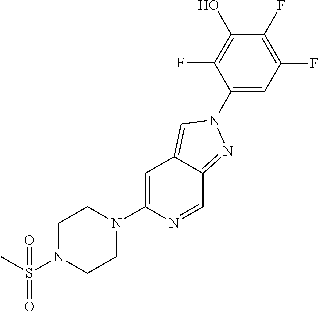 [SMILES: CS(=O)(=O)N1CCN(c2cc3cn(-c4cc(F)c(F)c(O)c4F)nc3cn2)CC1] | 2,3,6-Trifluoro-5-(5-(4- (methylsulfonyl)piperazin-1-yl)-2H- pyrazolo[3,4-c]pyridine-2-yl)phenol | 428.0 |
| 19.31 (1) | 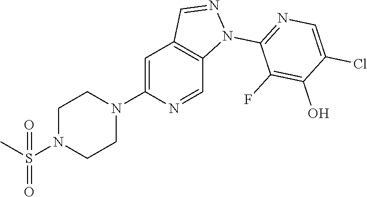 [SMILES: CS(=O)(=O)N1CCN(c2cc3cnn(-c4ncc(Cl)c(O)c4F)c3cn2)CC1] | 5-Chloro-3-fluoro-2-(5-(4- (methylsulfonyl)piperazin-1-yl)-1H- pyrazolo[3,4-c]pyridine-1- yl)pyridine-4-ol | 426.9 |
| 19.32 (1) | 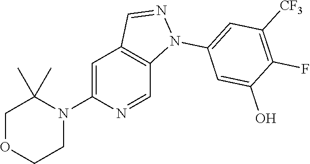 [SMILES: CC1(C)COCCN1c1cc2cnn(-c3cc(O)c(F)c(C(F)(F)F)c3)c2cn1] | 5-(5-(3,3-Dimethylmorpholino)-1H- pyrazolo[3,4-c]pyridine-1-yl)-2- fluoro-3-(trifluoromethyl)phenol | 411.2 |
| 19.33 (1) | 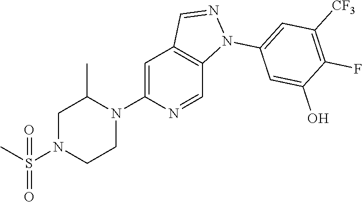 [SMILES: CC1CN(S(C)(=O)=O)CCN1c1cc2cnn(-c3cc(O)c(F)c(C(F)(F)F)c3)c2cn1] | 2-Fluoro-5-(5-(2-methyl-4- (methylsulfonyl)piperazin-1-yl)-1H- pyrazolo[3,4-c]pyridine-1-yl)-3- (trifluoromethyl)phenol | 474.2 |
| 19.34 (1,2) | 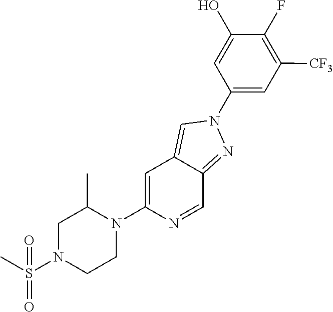 [SMILES: CC1CN(S(C)(=O)=O)CCN1c1cc2cn(-c3cc(O)c(F)c(C(F)(F)F)c3)nc2cn1] | 2-Fluoro-5-(5-(2-methyl-4- (methylsulfonyl)piperazin-1-yl)-2H- pyrazolo[3,4-c]pyridine-2-yl)-3- (trifluoromethyl)phenol | 474.1 |
| 19.35 (1,2) | 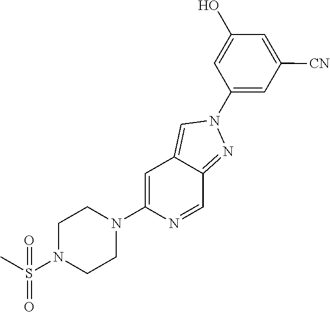 [SMILES: CS(=O)(=O)N1CCN(c2cc3cn(-c4cc(O)cc(C#N)c4)nc3cn2)CC1] | 3-Hydroxy-5-(5-(4- (methylsulfonyl)piperazin-1-yl)-2H- pyrazolo[3,4-c]pyridine-2- yl)benzonitrile | 399.1 |
| 19.36 (1) | 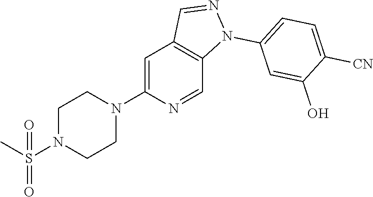 [SMILES: CS(=O)(=O)N1CCN(c2cc3cnn(-c4ccc(C#N)c(O)c4)c3cn2)CC1] | 2-Hydroxy-4-(5-(4- (methylsulfonyl)piperazin-1-yl)-1H- pyrazolo[3,4-c]pyridine-1- yl)benzonitrile | 399.0 |
| 19.37 (1,2) | 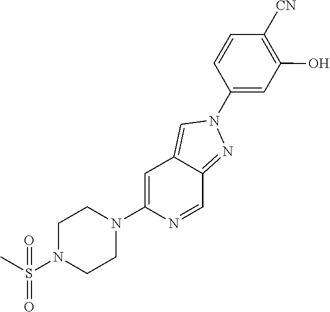 [SMILES: CS(=O)(=O)N1CCN(c2cc3cn(-c4ccc(C#N)c(O)c4)nc3cn2)CC1] | 2-Hydroxy-4-(5-(4- (methylsulfonyl)piperazin-1-yl)-2H- pyrazolo[3,4-c]pyridine-2- yl)benzonitrile | 399.0 |
| 19.38 (1) | 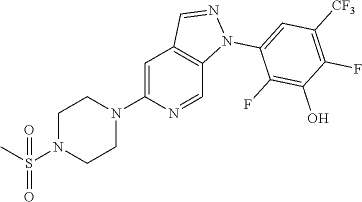 [SMILES: CS(=O)(=O)N1CCN(c2cc3cnn(-c4cc(C(F)(F)F)c(F)c(O)c4F)c3cn2)CC1] | 2,6-Difluoro-3-(5-(4- (methylsulfonyl)piperazin-1-yl)-1H- pyrazolo[3,4-c]pyridine-1-yl)-5- (trifluoromethyl)phenol | 478.0 |
| 19.39 (1) | 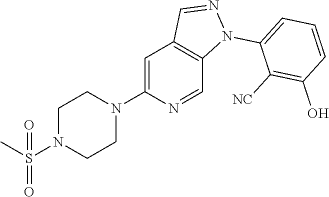 [SMILES: CS(=O)(=O)N1CCN(c2cc3cnn(-c4cccc(O)c4C#N)c3cn2)CC1] | 2-Hydroxy-6-(5-(4- (methylsulfonyl)piperazin-1-yl)-1H- pyrazolo[3,4-c]pyridine-1- yl)benzonitrile | 399.1 |
| 19.40 (1,2) | 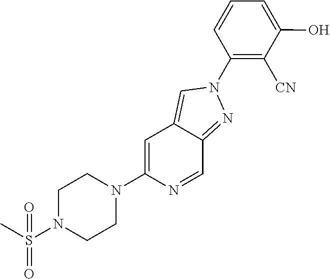 [SMILES: CS(=O)(=O)N1CCN(c2cc3cn(-c4cccc(O)c4C#N)nc3cn2)CC1] | 2-Hydroxy-6-(5-(4- (methylsulfonyl)piperazin-1-yl)-2H- pyrazolo[3,4-c]pyridine-2- yl)benzonitrile | 399.0 |
| 19.41 (1) | 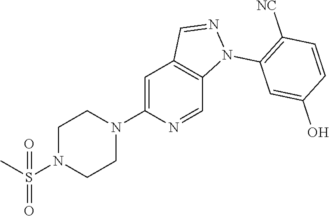 [SMILES: CS(=O)(=O)N1CCN(c2cc3cnn(-c4cc(O)ccc4C#N)c3cn2)CC1] | 4-Hydroxy-2-(5-(4- (methylsulfonyl)piperazin-1-yl)-1H- pyrazolo[3,4-c]pyridine-1- yl)benzonitrile | 399.1 |
| 19.42 (1) | 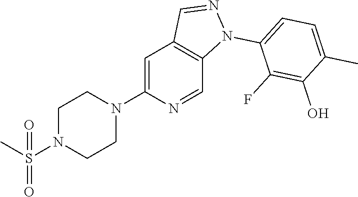 | 2-Fluoro-6-methyl-3-(5-(4- (methylsulfonyl)piperazin-1-yl)-1H- pyrazolo[3,4-c]pyridine-1-yl)phenol | 406.0 |
| 19.43 (1) | 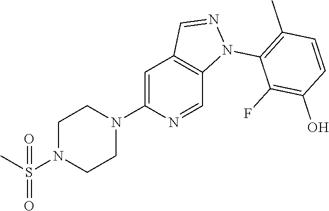 [SMILES: Cc1ccc(O)c(F)c1-n1ncc2cc(N3CCN(S(C)(=O)=O)CC3)ncc21] | 2-Fluoro-4-methyl-3-(5-(4- (methylsulfonyl)piperazin-1-yl)-1H- pyrazolo[3,4-c]pyridine-1-yl)phenol | 406.0 |
| 19.44 (1) | 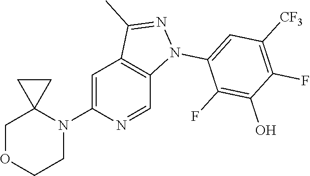 [SMILES: Cc1nn(-c2cc(C(F)(F)F)c(F)c(O)c2F)c2cnc(N3CCOCC34CC4)cc12] | 2,6-Difluoro-3-(3-methyl-5-(7-oxa-4- azaspiro[2.5]octan-4-yl)-1H- pyrazolo[3,4-c]pyridine-1-yl)-5- (trifluoromethyl)phenol | 441.2 |
| 19.45 (1) | 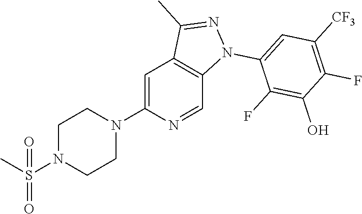 [SMILES: Cc1nn(-c2cc(C(F)(F)F)c(F)c(O)c2F)c2cnc(N3CCN(S(C)(=O)=O)CC3)cc12] | 2,6-Difluoro-3-(3-methyl-5-(4- (methylsulfonyl)piperazin-1-yl)-1H- pyrazolo[3,4-c]pyridine-1-yl)-5- (trifluoromethyl)phenol | 492.2 |
| 19.46 (2) | 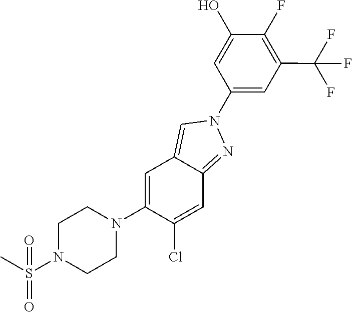 [SMILES: CS(=O)(=O)N1CCN(c2cc3cn(-c4cc(O)c(F)c(C(F)(F)F)c4)nc3cc2Cl)CC1] | 5-(6-Chloro-5-(4- (methylsulfonyl)piperazin-1-yl)-2H- indazol-2-yl)-2-fluoro-3- (trifluoromethyl)phenol | 492.9 |
| 19.47 | 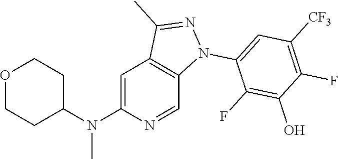 [SMILES: Cc1nn(-c2cc(C(F)(F)F)c(F)c(O)c2F)c2cnc(N(C)C3CCOCC3)cc12] | 2,6-Difluoro-3-(3-methyl-5- (methyl(tetrahydro-2H-pyran-4- yl)amino)-1H-pyrazolo[3,4- c]pyridin-1-yl)-5- (trifluoromethyl)phenol | 443.1 |
| 19.48 | 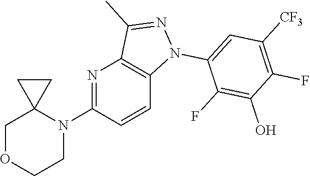 [SMILES: Cc1nn(-c2cc(C(F)(F)F)c(F)c(O)c2F)c2ccc(N3CCOCC34CC4)nc12] | 2,6-Difluoro-3-(3-methyl-5-(7-oxa-4- azaspiro[2.5]octan-4-yl)-1H- pyrazolo[4,3-b]pyridine-1-yl)-5- (trifluoromethyl)phenol | 441.1 |
| 19.49 | 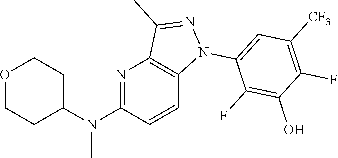 [SMILES: Cc1nn(-c2cc(C(F)(F)F)c(F)c(O)c2F)c2ccc(N(C)C3CCOCC3)nc12] | 2,6-Difluoro-3-(3-methyl-5- (methyl(tetrahydro-2H-pyran-4- yl)amino)-1H-pyrazolo[4,3-b] pyridine-1-yl)-5- (trifluoromethyl)phenol | 443.1 |
| 19.50 | 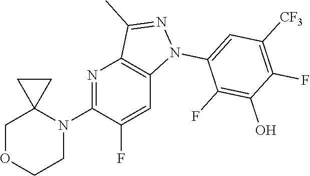 [SMILES: Cc1nn(-c2cc(C(F)(F)F)c(F)c(O)c2F)c2cc(F)c(N3CCOCC34CC4)nc12] | 2,6-Difluoro-3-(6-fluoro-3-methyl-5- (7-oxa-4-azaspiro[2.5]octan-4-yl)- 1H-pyrazolo[4,3-b]pyridine-1-yl)-5- (trifluoromethyl)phenol | 459.0 |
| 19.51 (3) | 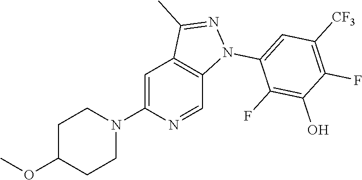 [SMILES: COC1CCN(c2cc3c(C)nn(-c4cc(C(F)(F)F)c(F)c(O)c4F)c3cn2)CC1] | 2,6-Difluoro-3-(5-(4- methoxypiperidin-1-yl)-3-methyl-1H- pyrazolo[3,4-c]pyridine-1-yl)-5- (trifluoromethyl)phenol | 443.3 |
| 19.52 (3) | 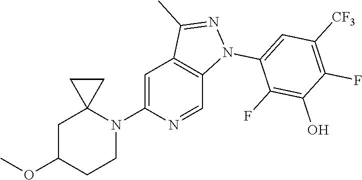 [SMILES: COC1CCN(c2cc3c(C)nn(-c4cc(C(F)(F)F)c(F)c(O)c4F)c3cn2)C2(CC2)C1] | 2,6-Difluoro-3-(5-(7-methoxy-4- azaspiro[2.5]octan-4-yl)-3-methyl- 1H-pyrazolo[3,4-c]pyridine-1-yl)-5- (trifluoromethyl)phenol | 469.2 |
| 19.53 (3) | 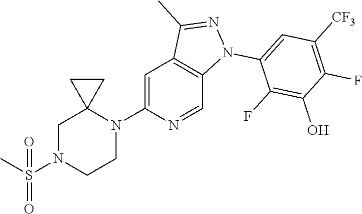 [SMILES: Cc1nn(-c2cc(C(F)(F)F)c(F)c(O)c2F)c2cnc(N3CCN(S(C)(=O)=O)CC34CC4)cc12] | 2,6-Difluoro-3-(3-methyl-5-(7- (methylsulfonyl)-4,7- diazaspiro[2.5]octan-4-yl)-1H- pyrazolo[3,4-c]pyridine-1-yl)-5- (trifluoromethyl)phenol | 518.2 |
| 19.54 (3) | 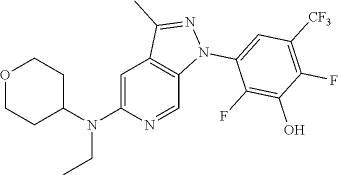 [SMILES: CCN(c1cc2c(C)nn(-c3cc(C(F)(F)F)c(F)c(O)c3F)c2cn1)C1CCOCC1] | 3-(5-(Ethyl(tetrahydro-2H-pyran-4- yl)amino)-3-methyl-1H-pyrazolo[3,4- c]pyridine-1-yl)-2,6-difluoro-5- (trifluoromethyl)phenol | 457.2 |
| 19.55 (3) | 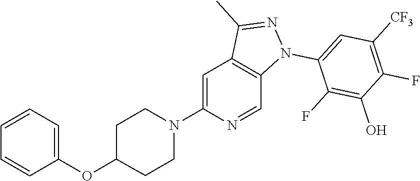 [SMILES: Cc1nn(-c2cc(C(F)(F)F)c(F)c(O)c2F)c2cnc(N3CCC(Oc4ccccc4)CC3)cc12] | 2,6-Difluoro-3-(3-methyl-5-(4- phenoxypiperidin-1-yl)-1H- pyrazolo[3,4-c]pyridine-1-yl)-5- (trifluoromethyl)phenol | 505.2 |
| 19.56 (3) | 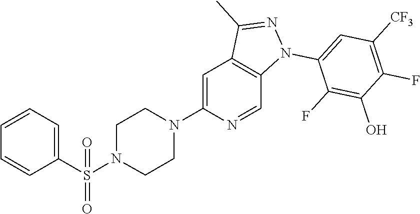 [SMILES: Cc1nn(-c2cc(C(F)(F)F)c(F)c(O)c2F)c2cnc(N3CCN(S(=O)(=O)c4ccccc4)CC3)cc12] | 2,6-Difluoro-3-(3-methyl-5-(4- (phenylsulfonyl)piperazin-1-yl)-1H- pyrazolo[3,4-c]pyridine-1-yl)-5- (trifluoromethyl)phenol | 554.2 |
| 19.57 (3) | 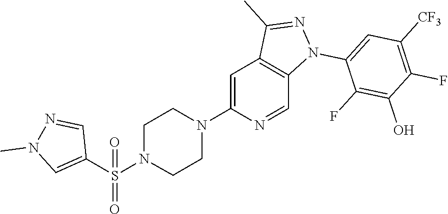 [SMILES: Cc1nn(-c2cc(C(F)(F)F)c(F)c(O)c2F)c2cnc(N3CCN(S(=O)(=O)c4cnn(C)c4)CC3)cc12] | 2,6-Difluoro-3-(3-methyl-5-(4-((1- methyl-1H-pyrazol-4- yl)sulfonyl)piperazin-1-yl)-1H- pyrazolo[3,4-c]pyridin-1-yl)-5- (trifluoromethyl)phenol | 558.1 |
| 19.58 (3) | 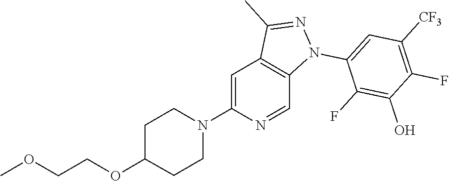 [SMILES: COCCOC1CCN(c2cc3c(C)nn(-c4cc(C(F)(F)F)c(F)c(O)c4F)c3cn2)CC1] | 2,6-Difluoro-3-(5-(4-(2- methoxyethoxy)piperidin-1-yl)-3- methyl-1H-pyrazolo[3,4-c]pyridin-1- yl)-5-(trifluoromethyl)phenol | 487.1 |
| 19.59 (3) | 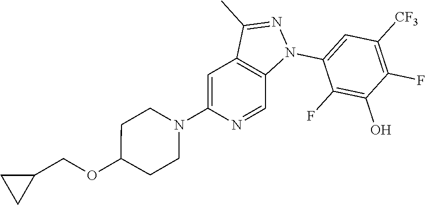 [SMILES: Cc1nn(-c2cc(C(F)(F)F)c(F)c(O)c2F)c2cnc(N3CCC(OCC4CC4)CC3)cc12] | 3-(5-(4- (Cyclopropylmethoxy)piperidin-1- yl)-3-methyl-1H-pyrazolo[3,4- c]pyridine-1-yl)-2,6-difluoro-5- (trifluoromethyl)phenol | 483.4 |
| 19.60 (3) | 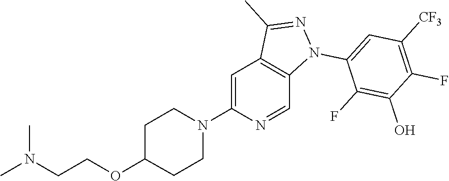 [SMILES: Cc1nn(-c2cc(C(F)(F)F)c(F)c(O)c2F)c2cnc(N3CCC(OCCN(C)C)CC3)cc12] | 3-(5-(4-(2- (Dimethylamino)ethoxy)piperidin-1- yl)-3-methyl-1H-pyrazolo[3,4- c]pyridine-1-yl)-2,6-difluoro-5- (trifluoromethyl)phenol | 500.2 |
| 19.61 | 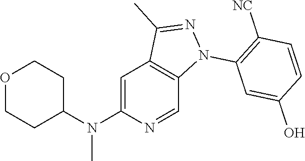 [SMILES: Cc1nn(-c2cc(O)ccc2C#N)c2cnc(N(C)C3CCOCC3)cc12] | 4-Hydroxy-2-(3-methyl-5- (methyl(tetrahydro-2H-pyran-4- yl)amino)-1H-pyrazolo[3,4- c]pyridin-1-yl)benzonitrile | 364.5 |
| 19.62 | 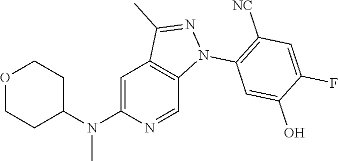 [SMILES: Cc1nn(-c2cc(O)c(F)cc2C#N)c2cnc(N(C)C3CCOCC3)cc12] | 5-Fluoro-4-hydroxy-2-(3-methyl-5- (methyl(tetrahydro-2H-pyran-4- yl)amino)-1H-pyrazolo[3,4- c]pyridin-1-yl)benzonitrile | 382.3 |
| 19.63 (3) | 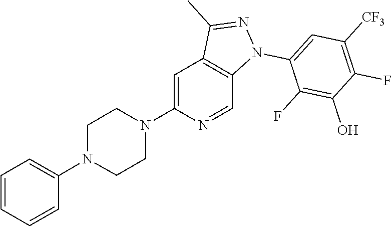 [SMILES: Cc1nn(-c2cc(C(F)(F)F)c(F)c(O)c2F)c2cnc(N3CCN(c4ccccc4)CC3)cc12] | 2,6-Difluoro-3-(3-methyl-5-(4- phenylpiperazin-1-yl)-1H- pyrazolo[3,4-c]pyridin-1-yl)-5- (trifluoromethyl)phenol | 490.4 |
| 19.64 (3) | 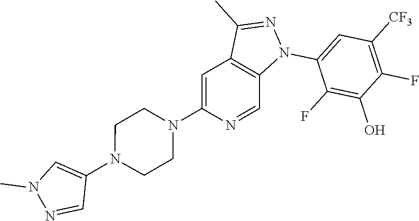 [SMILES: Cc1nn(-c2cc(C(F)(F)F)c(F)c(O)c2F)c2cnc(N3CCN(c4cnn(C)c4)CC3)cc12] | 2,6-Difluoro-3-(3-methyl-5-(4-(1- methyl-1H-pyrazol-4-yl)piperazin-1- yl)-1H-pyrazolo[3,4-c]pyridin-1-yl)- 5-(trifluoromethyl)phenol | 494.4 |
| 19.65 (3) | 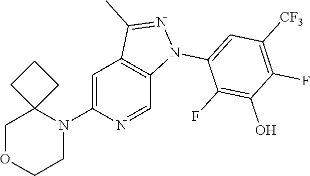 [SMILES: Cc1nn(-c2cc(C(F)(F)F)c(F)c(O)c2F)c2cnc(N3CCOCC34CCC4)cc12] | 2,6-Difluoro-3-(3-methyl-5-(8-oxa-5- azaspiro[3.5]nonan-5-yl)-1H- pyrazolo[3,4-c]pyridin-1-yl)-5- (trifluoromethyl)phenol | 455.3 |
| 19.66 (1,3) | 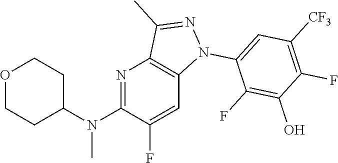 [SMILES: Cc1nn(-c2cc(C(F)(F)F)c(F)c(O)c2F)c2cc(F)c(N(C)C3CCOCC3)nc12] | 2,6-Difluoro-3-(6-fluoro-3-methyl-5- (methyl(tetrahydro-2H-pyran-4- yl)amino)-1H-pyrazolo[4,3- b]pyridin-1-yl)-5- (trifluoromethyl)phenol | 461.1 |
| 19.67 (1,3) | 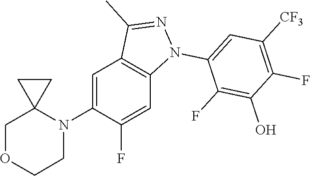 [SMILES: Cc1nn(-c2cc(C(F)(F)F)c(F)c(O)c2F)c2cc(F)c(N3CCOCC34CC4)cc12] | 2,6-Difluoro-3-(6-fluoro-3-methyl-5- (7-oxa-4-azaspiro[2.5]octan-4-yl)- 1H-indazol-1-yl)-5- (trifluoromethyl)phenol | 458.4 |
| 19.68 (1,3) | 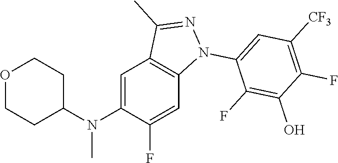 [SMILES: Cc1nn(-c2cc(C(F)(F)F)c(F)c(O)c2F)c2cc(F)c(N(C)C3CCOCC3)cc12] | 2,6-Difluoro-3-(6-fluoro-3-methyl-5- (methyl(tetrahydro-2H-pyran-4- yl)amino)-1H-indazol-1-yl)-5- (trifluoromethyl)phenol | 460.2 |
| 19.69 (1,3) | 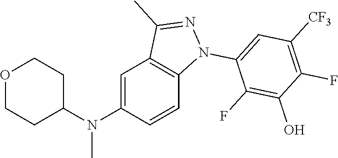 [SMILES: Cc1nn(-c2cc(C(F)(F)F)c(F)c(O)c2F)c2ccc(N(C)C3CCOCC3)cc12] | 2,6-Difluoro-3-(3-methyl-5- (methyl(tetrahydro-2H-pyran-4- yl)amino)-1H-indazol-1-yl)-5- (trifluoromethyl)phenol | 442.3 |
| 19.70 (1,3) | 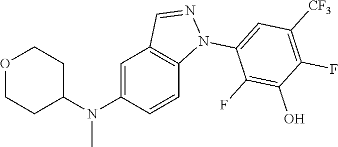 [SMILES: CN(c1ccc2c(cnn2-c2cc(C(F)(F)F)c(F)c(O)c2F)c1)C1CCOCC1] | 2,6-Difluoro-3-(5- (methyl(tetrahydro-2H-pyran-4- yl)amino)-1H-indazol-1-yl)-5- (trifluoromethyl)phenol | 428.5 |
| 19.71 (1,3) | 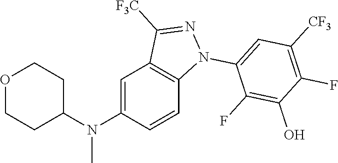 [SMILES: CN(c1ccc2c(c1)c(C(F)(F)F)nn2-c1cc(C(F)(F)F)c(F)c(O)c1F)C1CCOCC1] | 2,6-Difluoro-3-(5- (methyl(tetrahydro-2H-pyran-4- yl)amino)-3-(trifluoromethyl)-1H- indazol-1-yl)-5- (trifluoromethyl)phenol | 496.5 |
| 19.72 (1,3) | 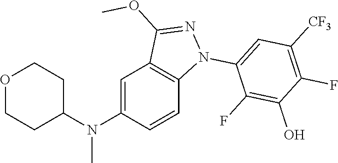 [SMILES: COc1nn(-c2cc(C(F)(F)F)c(F)c(O)c2F)c2ccc(N(C)C3CCOCC3)cc12] | 2,6-Difluoro-3-(3-methoxy-5- (methyl(tetrahydro-2H-pyran-4- yl)amino)-1H-indazol-1-yl)-5- (trifluoromethyl)phenol | 458.3 |
| 19.73 (1,3) | 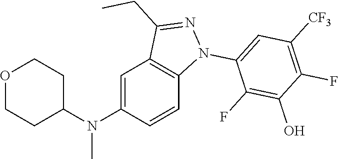 [SMILES: CCc1nn(-c2cc(C(F)(F)F)c(F)c(O)c2F)c2ccc(N(C)C3CCOCC3)cc12] | 3-(3-Ethyl-5-(methyl(tetrahydro-2H- pyran-4-yl)amino)-1H-indazol-1-yl)- 2,6-difluoro-5- (trifluoromethyl)phenol | 456.3 |
| 19.74 (1,3) | 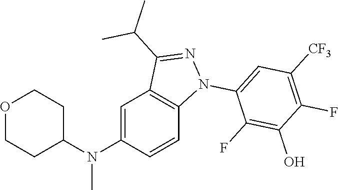 [SMILES: CC(C)c1nn(-c2cc(C(F)(F)F)c(F)c(O)c2F)c2ccc(N(C)C3CCOCC3)cc12] | 2,6-Difluoro-3-(3-isopropyl-5- (methyl(tetrahydro-2H-pyran-4- yl)amino)-1H-indazol-1-yl)-5- (trifluoromethyl)phenol | 470.4 |
| 19.75 (1,3) | 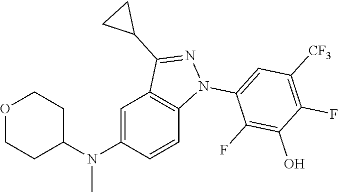 [SMILES: CN(c1ccc2c(c1)c(C1CC1)nn2-c1cc(C(F)(F)F)c(F)c(O)c1F)C1CCOCC1] | 3-(3-Cyclopropyl-5- (methyl(tetrahydro-2H-pyran-4- yl)amino)-1H-indazol-1-yl)-2,6- difluoro-5-(trifluoromethyl)phenol | 468.4 |
| 19.76 (1,3) | 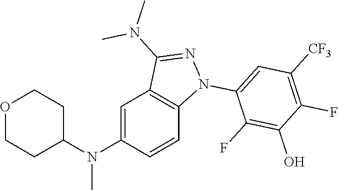 [SMILES: CN(C)c1nn(-c2cc(C(F)(F)F)c(F)c(O)c2F)c2ccc(N(C)C3CCOCC3)cc12] | 3-(3-(Dimethylamino)-5- (methyl(tetrahydro-2H-pyran-4- yl)amino)-1H-indazol-1-yl)-2,6- difluoro-5-(trifluoromethyl)phenol | 471.5 |
| 19.77 (1,3) | 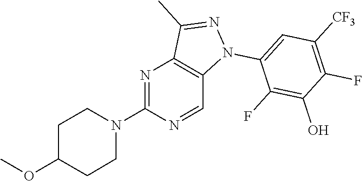 [SMILES: COC1CCN(c2ncc3c(n2)c(C)nn3-c2cc(C(F)(F)F)c(F)c(O)c2F)CC1] | 2,6-Difluoro-3-(5-(4- methoxypiperidin-1-yl)-3-methyl-1H- pyrazolo[4,3-d]pyrimidin-1-yl)-5- (trifluoromethyl)phenol | 444.2 |
| 19.78 (1,3) | 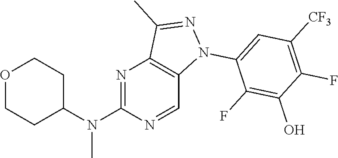 [SMILES: Cc1nn(-c2cc(C(F)(F)F)c(F)c(O)c2F)c2cnc(N(C)C3CCOCC3)nc12] | 2,6-Difluoro-3-(3-methyl-5- (methyl(tetrahydro-2H-pyran-4- yl)amino)-1H-pyrazolo[4,3- d]pyrimidin-1-yl)-5- (trifluoromethyl)phenol | 444.2 |
| 19.79 (1,3) | 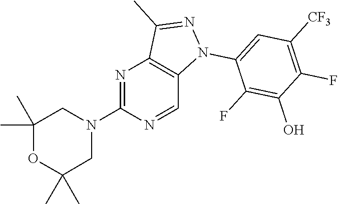 [SMILES: Cc1nn(-c2cc(C(F)(F)F)c(F)c(O)c2F)c2cnc(N3CC(C)(C)OC(C)(C)C3)nc12] | 2,6-Difluoro-3-(3-methyl-5-(2,2,6,6- tetramethylmorpholino)-1H- pyrazolo[4,3-d]pyrimidin-1-yl)-5- (trifluoromethyl)phenol | 472.1 |
| 19.80 (1,3) | 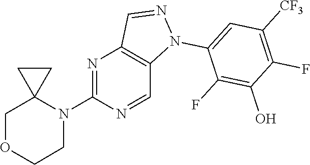 [SMILES: Oc1c(F)c(-n2ncc3nc(N4CCOCC45CC5)ncc32)cc(C(F)(F)F)c1F] | 3-(5-(7-Oxa-4-azaspiro[2.5]octan-4- yl)-1H-pyrazolo[4,3-d]pyrimidin-1- yl)-2,6-difluoro-5- (trifluoromethyl)phenol | 428.1 |
| 19.81 (1,3) | 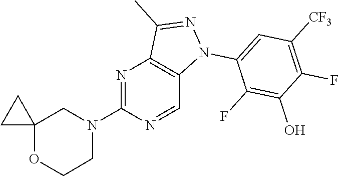 [SMILES: Cc1nn(-c2cc(C(F)(F)F)c(F)c(O)c2F)c2cnc(N3CCOC4(CC4)C3)nc12] | 2,6-Difluoro-3-(3-methyl-5-(4-oxa-7- azaspiro[2.5]octan-7-yl)-1H- pyrazolo[4,3-d]pyrimidin-1-yl)-5- (trifluoromethyl)phenol | 442.1 |
| 19.82 (1,3) | 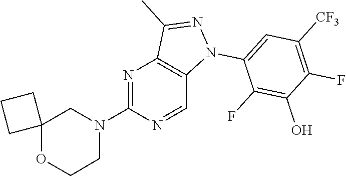 [SMILES: Cc1nn(-c2cc(C(F)(F)F)c(F)c(O)c2F)c2cnc(N3CCOC4(CCC4)C3)nc12] | 2,6-Difluoro-3-(3-methyl-5-(5-oxa-8- azaspiro[3.5]nonan-8-yl)-1H- pyrazolo[4,3-d]pyrimidin-1-yl)-5- (trifluoromethyl)phenol | 456.1 |
| 19.83 (1,3) | 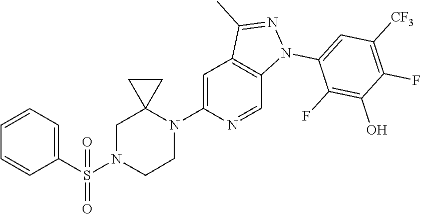 [SMILES: Cc1nn(-c2cc(C(F)(F)F)c(F)c(O)c2F)c2cnc(N3CCN(S(=O)(=O)c4ccccc4)CC34CC4)cc12] | 2,6-Difluoro-3-(3-methyl-5-(7- (phenylsulfonyl)-4,7- diazaspiro[2.5]octan-4-yl)-1H- pyrazolo[3,4-c]pyridin-1-yl)-5- (trifluoromethyl)phenol | 580.4 |
| 19.84 (1,3) | 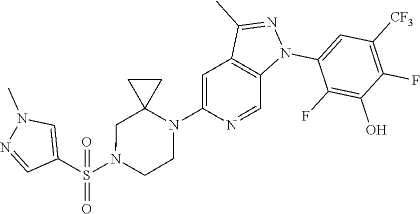 [SMILES: Cc1nn(-c2cc(C(F)(F)F)c(F)c(O)c2F)c2cnc(N3CCN(S(=O)(=O)c4cnn(C)c4)CC34CC4)cc12] | 2,6-Difluoro-3-(3-methyl-5-(7-((1- methyl-1H-pyrazol-4-yl)sulfonyl)- 4,7-diazaspiro[2.5]octan-4-yl)-1H- pyrazolo[3,4-c]pyridin-1-yl)-5- (trifluoromethyl)phenol | 584.5 |
| 19.85 (1) | 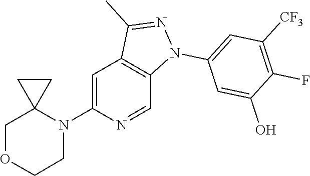 [SMILES: Cc1nn(-c2cc(O)c(F)c(C(F)(F)F)c2)c2cnc(N3CCOCC34CC4)cc12] | 2-Fluoro-5-(3-methyl-5-(7-oxa-4- azaspiro[2.5]octan-4-yl)-1H- pyrazolo[3,4-c]pyridin-1-yl)-3- (trifluoromethyl)phenol | 423.5 |
| 19.86 (1) | 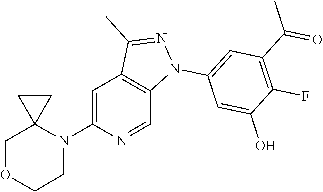 [SMILES: CC(=O)c1cc(-n2nc(C)c3cc(N4CCOCC45CC5)ncc32)cc(O)c1F] | 1-(2-Fluoro-3-hydroxy-5-(3-methyl- 5-(7-oxa-4-azaspiro[2.5]octan-4-yl)- 1H-pyrazolo[3,4-c]pyridin-1- yl)phenyl)ethanone | 397.3 |
| 19.87 (1,3) | 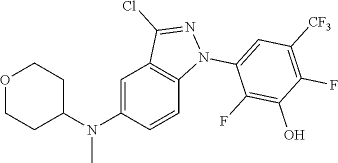 [SMILES: CN(c1ccc2c(c1)c(Cl)nn2-c1cc(C(F)(F)F)c(F)c(O)c1F)C1CCOCC1] | 3-(3-Chloro-5-(methyl(tetrahydro- 2H-pyran-4-yl)amino)-1H-indazol-1- yl)-2,6-difluoro-5- (trifluoromethyl)phenol | 462.3 |
| 19.88 (1,3) | 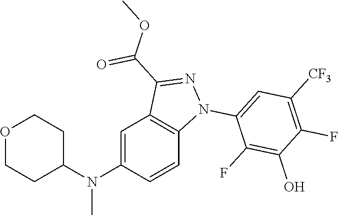 [SMILES: COC(=O)c1nn(-c2cc(C(F)(F)F)c(F)c(O)c2F)c2ccc(N(C)C3CCOCC3)cc12] | Methyl 1-(2,4-difluoro-3-hydroxy-5- (trifluoromethyl)phenyl)-5- (methyl(tetrahydro-2H-pyran-4- yl)amino)-1H-indazole-3-carboxylate | 486.4 |
| 19.89 (4) | 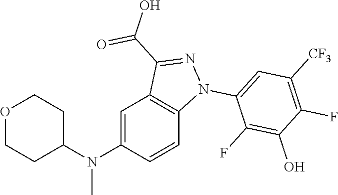 [SMILES: CN(c1ccc2c(c1)c(C(=O)O)nn2-c1cc(C(F)(F)F)c(F)c(O)c1F)C1CCOCC1] | 1-(2,4-Difluoro-3-hydroxy-5- (trifluoromethyl)phenyl)-5- (methyl(tetrahydro-2H-pyran-4- yl)amino)-1H-indazole-3-carboxylic Acid | 472.5 |
| 19.90 (1,3) | 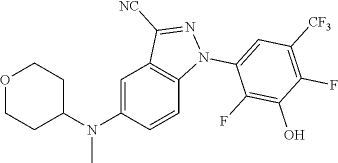 [SMILES: CN(c1ccc2c(c1)c(C#N)nn2-c1cc(C(F)(F)F)c(F)c(O)c1F)C1CCOCC1] | 1-(2,4-Difluoro-3-hydroxy-5- (trifluoromethyl)phenyl)-5- (methyl(tetrahydro-2H-pyran-4- yl)amino)-1H-indazole-3-carbonitrile | 453.3 |
| 19.91 (5) | 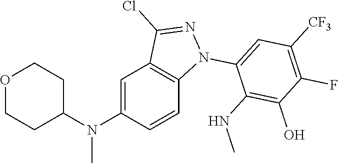 [SMILES: CNc1c(-n2nc(Cl)c3cc(N(C)C4CCOCC4)ccc32)cc(C(F)(F)F)c(F)c1O] | 3-(3-Chloro-5-(methyl(tetrahydro- 2H-pyran-4-yl)amino)-1H-indazol-1- yl)-6-fluoro-2-(methylamino)-5- (trifluoromethyl)phenol | 473.4 |
| 19.92 (1) | 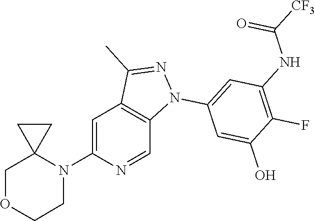 [SMILES: Cc1nn(-c2cc(O)c(F)c(NC(=O)C(F)(F)F)c2)c2cnc(N3CCOCC34CC4)cc12] | 2,2,2-Trifluoro-N-(2-fluoro-3- hydroxy-5-(3-methyl-5-(7-oxa-4- azaspiro[2.5]octan-4-yl)-1H- pyrazolo[3,4-c]pyridin-1- yl)phenyl)acetamide | 466.3 |
| 19.93 (6) | 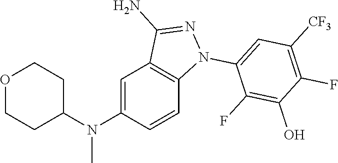 [SMILES: CN(c1ccc2c(c1)c(N)nn2-c1cc(C(F)(F)F)c(F)c(O)c1F)C1CCOCC1] | 3-(3-Amino-5-(methyl(tetrahydro- 2H-pyran-4-yl)amino)-1H-indazol-1- yl)-2,6-difluoro-5- (trifluoromethyl)phenol | 443.4 |
| 19.94 (1,3) | 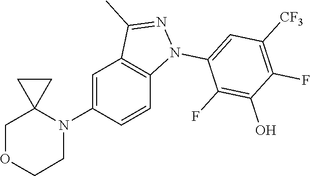 [SMILES: Cc1nn(-c2cc(C(F)(F)F)c(F)c(O)c2F)c2ccc(N3CCOCC34CC4)cc12] | 2,6-Difluoro-3-(3-methyl-5-(7-oxa-4- azaspiro[2.5]octan-4-yl)-1H-indazol- 1-yl)-5-(trifluoromethyl)phenol | 440.4 |
| 19.95 (1,3) | 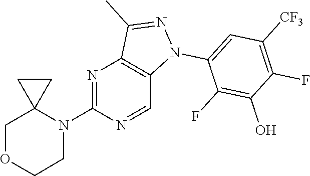 [SMILES: Cc1nn(-c2cc(C(F)(F)F)c(F)c(O)c2F)c2cnc(N3CCOCC34CC4)nc12] | 2,6-Difluoro-3-(3-methyl-5-(7-oxa-4- azaspiro[2.5]octan-4-yl)-1H- pyrazolo[4,3-d]pyrimidin-1-yl)-5- (trifluoromethyl)phenol | 442.4 |
| 19.96 (1,7) | 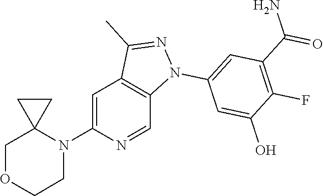 [SMILES: Cc1nn(-c2cc(O)c(F)c(C(N)=O)c2)c2cnc(N3CCOCC34CC4)cc12] | 2-Fluoro-3-hydroxy-5-(3-methyl-5- (7-oxa-4-azaspiro[2.5]octan-4-yl)- 1H-pyrazolo[3,4-c]pyridin-1- yl)benzamide | 398.5 |
| 19.97 (8) | 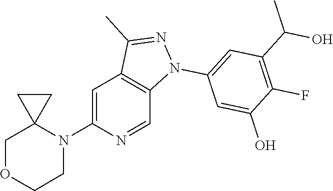 [SMILES: Cc1nn(-c2cc(O)c(F)c(C(C)O)c2)c2cnc(N3CCOCC34CC4)cc12] | 2-Fluoro-3-(1-hydroxyethyl)-5-(3- methyl-5-(7-oxa-4- azaspiro[2.5]octan-4-yl)-1H- pyrazolo[3,4-c]pyridin-1-yl)phenol | 399.5 |
| 19.98 (1) | 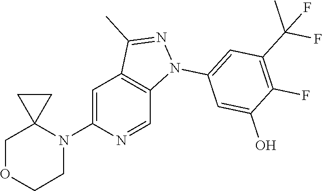 [SMILES: Cc1nn(-c2cc(O)c(F)c(C(C)(F)F)c2)c2cnc(N3CCOCC34CC4)cc12] | 3-(1,1-Difluoroethyl)-2-fluoro-5-(3- methyl-5-(7-oxa-4- azaspiro[2.5]octan-4-yl)-1H- pyrazolo[3,4-c]pyridin-1-yl)phenol | 419.4 |
| 19.99 (1,7) | 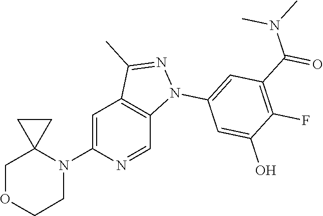 [SMILES: Cc1nn(-c2cc(O)c(F)c(C(=O)N(C)C)c2)c2cnc(N3CCOCC34CC4)cc12] | 2-Fluoro-3-hydroxy-N,N-dimethyl-5- (3-methyl-5-(7-oxa-4- azaspiro[2.5]octan-4-yl)-1H- pyrazolo[3,4-c]pyridin-1- yl)benzamide | 426.5 |
| 19.100 (1,3) | 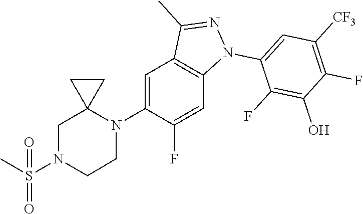 [SMILES: Cc1nn(-c2cc(C(F)(F)F)c(F)c(O)c2F)c2cc(F)c(N3CCN(S(C)(=O)=O)CC34CC4)cc12] | 2,6-Difluoro-3-(6-fluoro-3-methyl-5- (7-(methylsulfonyl)-4,7- diazaspiro[2.5]octan-4-yl)-1H- indazol-1-yl)-5- (trifluoromethyl)phenol | 535.4 |
| 19.101 (1,3) | 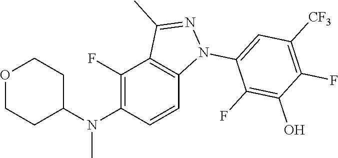 [SMILES: Cc1nn(-c2cc(C(F)(F)F)c(F)c(O)c2F)c2ccc(N(C)C3CCOCC3)c(F)c12] | 2,6-Difluoro-3-(4-fluoro-3-methyl-5- (methyl(tetrahydro-2H-pyran-4- yl)amino)-1H-indazol-1-yl)-5- (trifluoromethyl)phenol | 460.1 |
| 19.102 (1,3) | 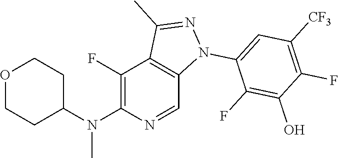 [SMILES: Cc1nn(-c2cc(C(F)(F)F)c(F)c(O)c2F)c2cnc(N(C)C3CCOCC3)c(F)c12] | 2,6-Difluoro-3-(4-fluoro-3-methyl-5- (methyl(tetrahydro-2H-pyran-4- yl)amino)-1H-pyrazolo[3,4- c]pyridin-1-yl)-5- (trifluoromethyl)phenol | 461.1 |
| 19.103 (1,9) | 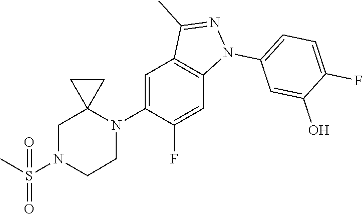 [SMILES: Cc1nn(-c2ccc(F)c(O)c2)c2cc(F)c(N3CCN(S(C)(=O)=O)CC34CC4)cc12] | 2-Fluoro-5-(6-fluoro-3-methyl-5-(7- (methylsulfonyl)-4,7- diazaspiro[2.5]octan-4-yl)-1H- indazol-1-yl)phenol | 449.3 |
| 19.104 (1) | 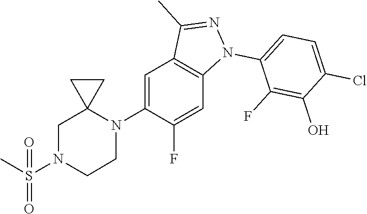 [SMILES: Cc1nn(-c2ccc(Cl)c(O)c2F)c2cc(F)c(N3CCN(S(C)(=O)=O)CC34CC4)cc12] | 6-Chloro-2-fluoro-3-(6-fluoro-3- methyl-5-(7-(methylsulfonyl)-4,7- diazaspiro[2.5]octan-4-yl)-1H- indazol-1-yl)phenol | 483.3 |
| 19.105 (1,3) | 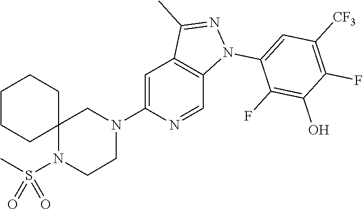 [SMILES: Cc1nn(-c2cc(C(F)(F)F)c(F)c(O)c2F)c2cnc(N3CCN(S(C)(=O)=O)C4(CCCCC4)C3)cc12] | 2,6-Difluoro-3-(3-methyl-5-(1- (methylsulfonyl)-1,4- diazaspiro[5.5]undecan-4-yl)-1H- pyrazolo[3,4-c]pyridin-1-yl)-5- (trifluoromethyl)phenol | 560.5 |
| 19.106 (6) | 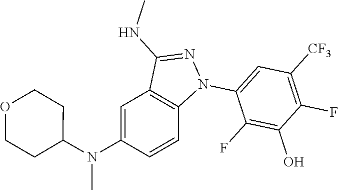 [SMILES: CNc1nn(-c2cc(C(F)(F)F)c(F)c(O)c2F)c2ccc(N(C)C3CCOCC3)cc12] | 2,6-Difluoro-3-(5- (methyl(tetrahydro-2H-pyran-4- yl)amino)-3-(methylamino)-1H- indazol-1-yl)-5- (trifluoromethyl)phenol | 457.4 |
| 19.107 (1,3) | 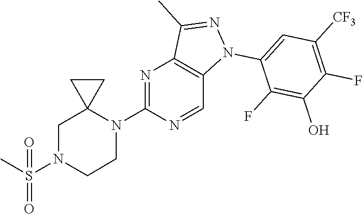 [SMILES: Cc1nn(-c2cc(C(F)(F)F)c(F)c(O)c2F)c2cnc(N3CCN(S(C)(=O)=O)CC34CC4)nc12] | 2,6-Difluoro-3-(3-methyl-5-(7- (methylsulfonyl)-4,7- diazaspiro[2.5]octan-4-yl)-1H- pyrazolo[4,3-d]pyrimidin-1-yl)-5- (trifluoromethyl)phenol | 519.4 |
| 19.108 (1,3) | 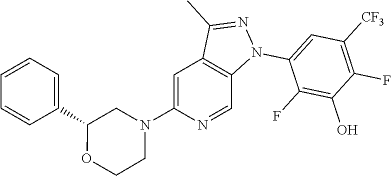 [SMILES: Cc1nn(-c2cc(C(F)(F)F)c(F)c(O)c2F)c2cnc(N3CCO[C@H](c4ccccc4)C3)cc12] | (R)-2,6-Difluoro-3-(3-methyl-5-(2- phenylmorpholino)-1H-pyrazolo[3,4- c]pyridin-1-yl)-5- (trifluoromethyl)phenol | 491.5 |
| 19.109 (1,3) | 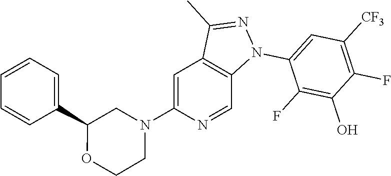 [SMILES: Cc1nn(-c2cc(C(F)(F)F)c(F)c(O)c2F)c2cnc(N3CCO[C@@H](c4ccccc4)C3)cc12] | (S)-2,6-Difluoro-3-(3-methyl-5-(2- phenylmorpholino)-1H-pyrazolo[3,4- c]pyridin-1-yl)-5- (trifluoromethyl)phenol | 491.4 |
| 19.110 (1) | 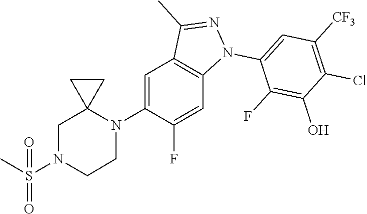 [SMILES: Cc1nn(-c2cc(C(F)(F)F)c(Cl)c(O)c2F)c2cc(F)c(N3CCN(S(C)(=O)=O)CC34CC4)cc12] | 2-Chloro-6-fluoro-5-(6-fluoro-3- methyl-5-(7-(methylsulfonyl)-4,7- diazaspiro[2.5]octan-4-yl)-1H- indazol-1-yl)-3- (trifluoromethyl)phenol | 551.4 |
| 19.111 (10) | 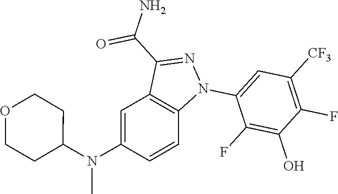 [SMILES: CN(c1ccc2c(c1)c(C(N)=O)nn2-c1cc(C(F)(F)F)c(F)c(O)c1F)C1CCOCC1] | 1-(2,4-Difluoro-3-hydroxy-5- (trifluoromethyl)phenyl)-5- (methyl(tetrahydro-2H-pyran-4- yl)amino)-1H-indazole-3- carboxamide | 471.4 |
| 19.112 (10) | 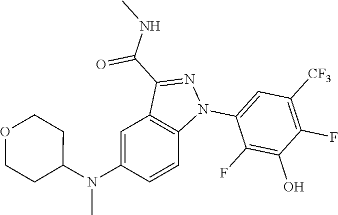 [SMILES: CNC(=O)c1nn(-c2cc(C(F)(F)F)c(F)c(O)c2F)c2ccc(N(C)C3CCOCC3)cc12] | 1-(2,4-Difluoro-3-hydroxy-5- (trifluoromethyl)phenyl)-N-methyl-5- (methyl(tetrahydro-2H-pyran-4- yl)amino)-1H-indazole-3- carboxamide | 485.4 |
| 19.113 (10) | 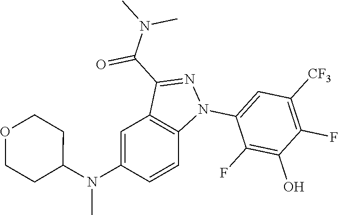 [SMILES: CN(C)C(=O)c1nn(-c2cc(C(F)(F)F)c(F)c(O)c2F)c2ccc(N(C)C3CCOCC3)cc12] | 1-(2,4-Difluoro-3-hydroxy-5- (trifluoromethyl)phenyl)-N,N- dimethyl-5-(methyl(tetrahydro-2H- pyran-4-yl)amino)-1H-indazole-3- carboxamide | 499.5 |
| 19.114 (1,3) | 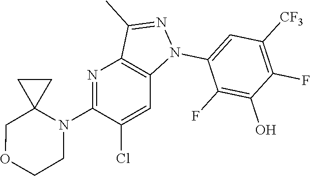 [SMILES: Cc1nn(-c2cc(C(F)(F)F)c(F)c(O)c2F)c2cc(Cl)c(N3CCOCC34CC4)nc12] | 3-(6-Chloro-3-methyl-5-(7-oxa-4- azaspiro[2.5]octan-4-yl)-1H- pyrazolo[4,3-b]pyridin-1-yl)-2,6- difluoro-5-(trifluoromethyl)phenol | 475.0 |
| 19.115 (1,3) | 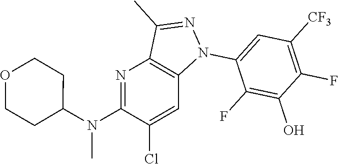 [SMILES: Cc1nn(-c2cc(C(F)(F)F)c(F)c(O)c2F)c2cc(Cl)c(N(C)C3CCOCC3)nc12] | 3-(6-Chloro-3-methyl-5- (methyl(tetrahydro-2H-pyran-4- yl)amino)-1H-pyrazolo[4,3- b]pyridin-1-yl)-2,6-difluoro-5- (trifluoromethyl)phenol | 477.0 |
| 19.116 (1,3) | 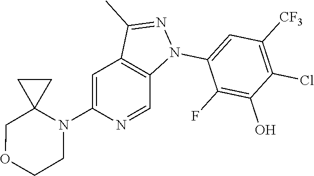 [SMILES: Cc1nn(-c2cc(C(F)(F)F)c(Cl)c(O)c2F)c2cnc(N3CCOCC34CC4)cc12] | 2-Chloro-6-fluoro-5-(3-methyl-5-(7- oxa-4-azaspiro[2.5]octan-4-yl)-1H- pyrazolo[3,4-c]pyridin-1-yl)-3- (trifluoromethyl)phenol | 457.3 |
| 19.117 (1,3) | 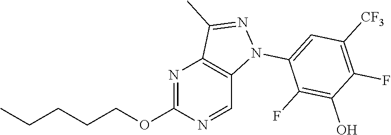 [SMILES: CCCCCOc1ncc2c(n1)c(C)nn2-c1cc(C(F)(F)F)c(F)c(O)c1F] | 2,6-Difluoro-3-(3-methyl-5- (pentyloxy)-1H-pyrazolo[4,3- d]pyrimidin-1-yl)-5- (trifluoromethyl)phenol | 417.5 |
| 19.118 (11) | 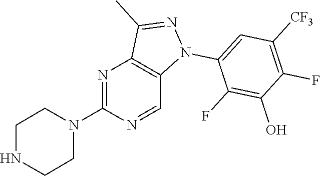 [SMILES: Cc1nn(-c2cc(C(F)(F)F)c(F)c(O)c2F)c2cnc(N3CCNCC3)nc12] | 2,6-Difluoro-3-(3-methyl-5- (piperazin-1-yl)-1H-pyrazolo[4,3- d]pyrimidin-1-yl)-5- (trifluoromethyl)phenol | 415.6 |
| 19.119 (1,3) | 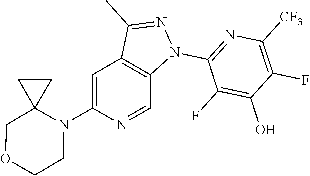 [SMILES: Cc1nn(-c2nc(C(F)(F)F)c(F)c(O)c2F)c2cnc(N3CCOCC34CC4)cc12] | 3,5-Difluoro-2-(3-methyl-5-(7-oxa-4- azaspiro[2.5]octan-4-yl)-1H- pyrazolo[3,4-c]pyridin-1-yl)-6- (trifluoromethyl)pyridin-4-ol | 442.1 |
| 19.120 (1) | 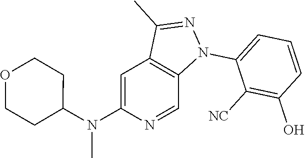 [SMILES: Cc1nn(-c2cccc(O)c2C#N)c2cnc(N(C)C3CCOCC3)cc12] | 2-Hydroxy-6-(3-methyl-5- (methyl(tetrahydro-2H-pyran-4- yl)amino)-1H-pyrazolo[3,4- c]pyridin-1-yl)benzonitrile | 364.5 |
| 19.121 (1) | 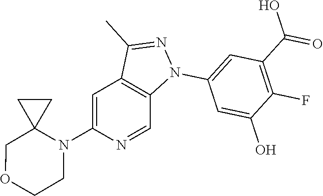 [SMILES: Cc1nn(-c2cc(O)c(F)c(C(=O)O)c2)c2cnc(N3CCOCC34CC4)cc12] | 2-Fluoro-3-hydroxy-5-(3-methyl-5- (7-oxa-4-azaspiro[2.5]octan-4-yl)- 1H-pyrazolo[3,4-c]pyridin-1- yl)benzoic Acid | 399.4 |
| 19.122 (10) | 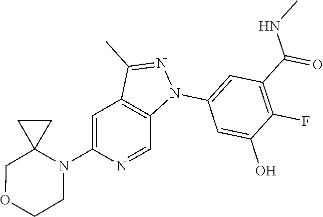 [SMILES: CNC(=O)c1cc(-n2nc(C)c3cc(N4CCOCC45CC5)ncc32)cc(O)c1F] | 2-Fluoro-3-hydroxy-N-methyl-5-(3- methyl-5-(7-oxa-4- azaspiro[2.5]octan-4-yl)-1H- pyrazolo[3,4-c]pyridin-1- yl)benzamide | 412.3 |
| 19.123 (1) | 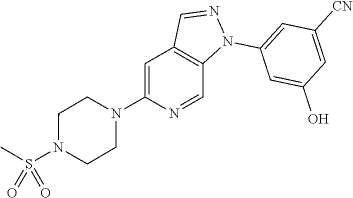 [SMILES: CS(=O)(=O)N1CCN(c2cc3cnn(-c4cc(O)cc(C#N)c4)c3cn2)CC1] | 3-Hydroxy-5-(5-(4- (methylsulfonyl)piperazin-1-yl)-1H- pyrazolo[3,4-c]pyridin-1- yl)benzonitrile | 399.1 |

Alternate conditions used: 1. CuI, trans-N,N′-dimethylcyclohexane-1,2-diamine, K₃PO₄, DMSO, 100-120° C., 45 min-overnight; 2. Isolated from the synthesis of N−1 isomer; 3. Iodide was used; 4. Ester hydrolysis from Compound 19.88 (1 N LiOH, THF, MeOH, rt, 2.5 h); 5. From Compound 19.87: methylamine HCl, DIEA, NMP, microwave, 130° C. for 30 min, 140° C. for 1.5 h; 6. From Compound 19.87: 0.4 M ammonia solution in dioxane or methylamine HCl, AdBrettPhos, AdBrettPhos Palladacycle Gen. 3, NaOtBu, dioxane, 80° C., 3 h-overnight; 7. Used 5-bromo-2-fluoro-3-hydroxybenzoic acid then HATU coupling (HATU, DIEA, DMF, appropriate amine, rt, 45 min); 8. Reduced from Compound 19.86: LiAlH₄, THF, 0° C., 5 min; 9. Used 3-bromo-2-chloro-6-fluorophenol (chloro reduced during Ullman); 10. From Compound 19.89 or 19.121: appropriate amine, HATU, DIEA, DMF, rt, 0.5 h-ON. 11. Synthesized from Intermediate 13.60.

### Compound 20

### 2-Fluoro-5-(5-(4-(methylsulfonyl)piperazin-1-yl)-1H-indazol-1-yl)phenol

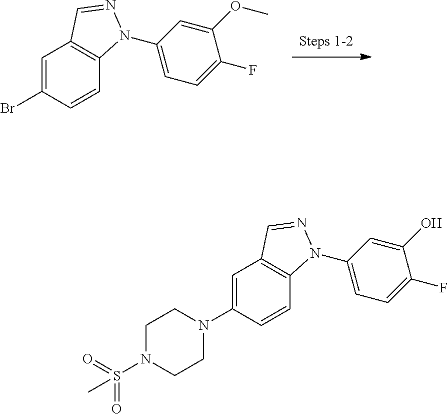 [SMILES: COc1cc(-n2ncc3cc(Br)ccc32)ccc1F ; CS(=O)(=O)N1CCN(c2ccc3c(cnn3-c3ccc(F)c(O)c3)c2)CC1]

### Step 1: 1-(4-Fluoro-3-methoxyphenyl)-5-(4-(methylsulfonyl)piperazin-1-yl)-1H-indazole

Pd₂(dba)₃ (71 mg, 0.077 mmol) and DavePhos (37 mg, 0.093 mmol) were added to a solution of Intermediate 14 (500 mg, 1.56 mmol), 1-(methylsulfonyl)piperazine (307 mg, 1.87 mmol), Cs₂CO₃ (761 mg, 2.34 mmol), and dioxane (8 mL)under N₂. The mixture was degassed with 3 vacuum/N₂ cycles, heated at 90° C. overnight, filtered through a Celite pad, and then concentrated. The residue was purified by silica gel chromatography (20-50% EtOAc/petroleum ether) to give 1-(4-fluoro-3-methoxyphenyl)-5-(4-(methylsulfonyl)piperazin-1-yl)-1H-indazole (490 mg, 77%) as a yellow solid. ¹H NMR (400 MHz, DMSO-d₆): δ 8.22 (s, 1H), 7.77-7.75 (m, 1H), 7.46 (d, 1H), 7.40 (d, 1H), 7.35-7.31 (m, 1H), 7.29-7.26 (m, 2H), 3.93 (s, 3H), 3.30-3.29 (m, 4H), 3.24-3.22 (m, 4H), 2.94 (s, 3H); LCMS: 405.2 [M+H]⁺.

### Step 2: 2-Fluoro-5-(5-(4-(methylsulfonyl)piperazin-1-yl)-1H-indazol-1-yl)phenol

Boron tribromide (929 mg, 3.71 mmol) was added dropwise to a solution of 1-(4-fluoro-3-methoxyphenyl)-5-(4-(methylsulfonyl)piperazin-1-yl)-1H-indazole (300 mg, 0.74 mmol) in DCM (3 mL) at −78° C. The mixture was stirred at −78° C. for 1 h, warmed to room temperature, and stirred for 1 h. Methanol (3 mL) was carefully added to the mixture at 0° C. The mixture was neutralized by adding saturated NaHCO₃ (˜20 mL) and extracted (3×10 mL DCM). The combined organic layers were washed (2×10 mL brine), dried (Na₂SO₄), filtered, and then concentrated. The residue was purified by prep-HPLC [water (0.04% NH₃·H₂O+10 mM NH₄HCO₃)-MeCN] to give 2-fluoro-5-(5-(4-(methylsulfonyl)piperazin-1-yl)-1H-indazol-1-yl)phenol (79 mg, 27%) as a white solid. ¹H NMR (400 MHz, DMSO-d₆): δ 9.20 (s, 1H), 8.18 (s, 1H), 7.69-7.67 (m, 1H), 7.34-7.26 (m, 4H), 7.13-7.11 (m, 1H), 3.30-3.22 (m, 8H), 2.94 (s, 3H); LCMS: 391.1 [M+H]⁺.

The Compounds below were synthesized in a similar manner to that described for Compound 20.

| Cmpd | Structure | Name | [M + H]⁺ |
|---|---|---|---|
| 20.01 | 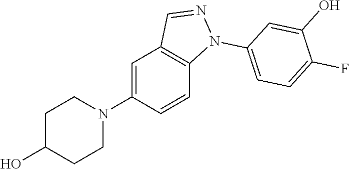 [SMILES: Oc1cc(-n2ncc3cc(N4CCC(O)CC4)ccc32)ccc1F] | 1-(1-(4-Fluoro-3-hydroxy- phenyl)-1H-indazol-5-yl)- piperidin-4-ol | 328.1 |
| 20.02 (1) | 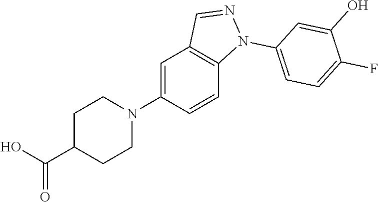 [SMILES: O=C(O)C1CCN(c2ccc3c(cnn3-c3ccc(F)c(O)c3)c2)CC1] | 1-(1-(4-Fluoro-3-hydroxy- phenyl)-1H-indazol-5-yl)- piperidine-4-carboxylic acid | 356.1 |
| 20.03 (2) | 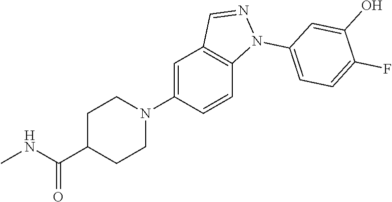 [SMILES: CNC(=O)C1CCN(c2ccc3c(cnn3-c3ccc(F)c(O)c3)c2)CC1] | 1-(1-(4-Fluoro-3-hydroxy- phenyl)-1H-indazol-5-yl)- N-methylpiperidine-4- carboxamide | 369.1 |
| 20.04 (2) | 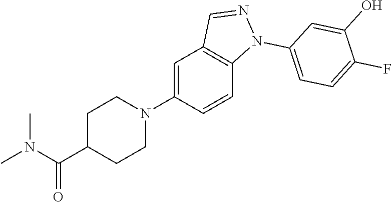 [SMILES: CN(C)C(=O)C1CCN(c2ccc3c(cnn3-c3ccc(F)c(O)c3)c2)CC1] | 1-(1-(4-Fluoro-3-hydroxy- phenyl)-1H-indazol-5-yl)- N,N-dimethylpiperidine-4- carboxamide | 383.1 |
| 20.05 | 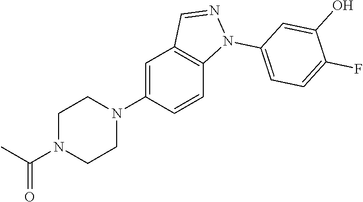 [SMILES: CC(=O)N1CCN(c2ccc3c(cnn3-c3ccc(F)c(O)c3)c2)CC1] | 1-(4-(1-(4-Fluoro-3- hydroxyphenyl)-1H- indazol-5-yl)piperazin-1- yl)ethan-1-one | 355.0 |

Alternate conditions used: 1. Synthesized using the following sequence: Step 1 (Pd(OAc)₂, Cs₂CO₃, tBuXPhos, dioxane, 90° C., overnight), hydrolysis (LiOH, H₂O, THF, rt, overnight), and then Step 2. 2. Synthesized from Compound 20.02 (hydrolysis step) using the following sequence: HATU coupling (MeNH₂·HCl or Me₂NH·HCl, HATU, DIPEA, CH₂Cl₂, rt, 3 h) then Compound 20, Step 2.

### Compound 21

### 2,3-Difluoro-5-(5-(4-(methylsulfonyl)piperazin-1-yl)-1H-indazol-1-yl)phenol

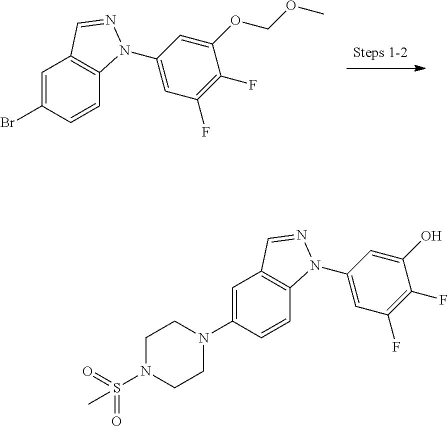

### Step 1: 1-(3,4-Difluoro-5-(methoxymethoxy)phenyl)-5-(4-(methylsulfonyl)piperazin-1-yl)-1H-indazole

Pd₂(dba)₃ (26 mg, 0.028 mmol) was added to a mixture of Intermediate 18 (300 mg, 0.57 mmol), 1-methylsulfonylpiperazine (187 mg, 1.14 mmol), RuPhos (27 mg, 0.057 mmol), NaO^{t}Bu (219 mg, 2.28 mmol), and toluene (3 mL) at room temperature. The mixture was degassed with 3 vacuum/N₂ cycles, heated at 100° C. overnight, allowed to cool to room temperature, poured into H₂O (20 mL), and then extracted (3×20 mL EtOAc). The combined organic layers were washed (100 mL brine), dried (Na₂SO₄), filtered, and then concentrated. The residue was purified by silica gel chromatography (50% EtOAc/petroleum ether) to give 1-(3,4-difluoro-5-(methoxymethoxy)phenyl)-5-(4-(methylsulfonyl)piperazin-1-yl)-1H-indazole (250 mg, 97%) as a yellow solid. ¹H NMR (400 MHz, DMSO-d₆): δ 8.25 (s, 1H), 7.80 (d, 1H), 7.54-7.44 (m, 2H), 7.37 (dd, 1H), 7.29 (d, 1H), 5.41 (s, 2H), 3.46 (s, 3H), 3.35-3.20 (m, 8H), 2.94 (s, 3H); LCMS: 453.2 [M+H]⁺.

### Step 2: 2,3-Difluoro-5-(5-(4-(methylsulfonyl)piperazin-1-yl)-1H-indazol-1-yl)phenol

Aqueous hydrochloric acid (3 M, 2.8 mL, 8.4 mmol) was added to a mixture of 1-(3,4-difluoro-5-(methoxymethoxy)phenyl)-5-(4-(methylsulfonyl)piperazin-1-yl)-1H-indazole (250 mg, 0.55 mmol), MeOH (0.5 mL), and THF (5 mL) at room temperature under N₂. The mixture was heated at 50° C. overnight, slowly poured into NaHCO₃ (10 mL), and then extracted (3×20 mL EtOAc). The combined organic layers were washed (10 mL brine), dried (Na₂SO₄), filtered, and then concentrated. The crude product was purified by prep-HPLC [water (0.04 HCl)/CH₃CN] to give 2,3-difluoro-5-(5-(4-(methylsulfonyl)piperazin-1-yl)-1H-indazol-N-yl)phenol (192 mg, 85%) as a white solid. ¹H NMR (400 MHz, DMSO-d₆): δ 10.93 (s, 1H), 8.24 (s, 1H), 7.78 (d, 1H), 7.40 (d, 1H), 7.33 (s, 1H), 7.29-7.19 (m, 2H), 3.34-3.29 (m, 8H), 2.95 (s, 3H); LCMS: 409.1 [M+H]⁺.

The compounds below were synthesized in a similar manner to that described for Compound 21.

| Cmpd | Structure | Name | [M + H]⁺ |
|---|---|---|---|
| 21.01 | 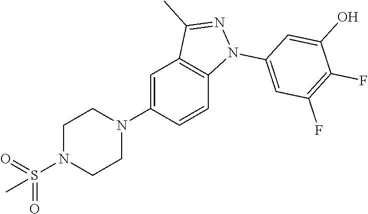 [SMILES: Cc1nn(-c2cc(O)c(F)c(F)c2)c2ccc(N3CCN(S(C)(=O)=O)CC3)cc12] | 2,3-Difluoro-5-(3-methyl-5-(4- (methylsulfonyl)piperazin-1- yl)-1H-indazol-1-yl)phenol | 423.1 |
| 21.02 | 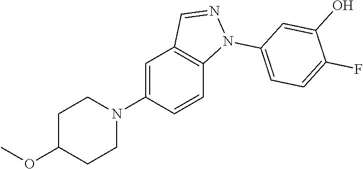 [SMILES: COC1CCN(c2ccc3c(cnn3-c3ccc(F)c(O)c3)c2)CC1] | 2-Fluoro-5-(5-(4-methoxy- piperidin-1-yl)-1H-indazol- 1-yl)phenol | 342.3 |
| 21.03 | 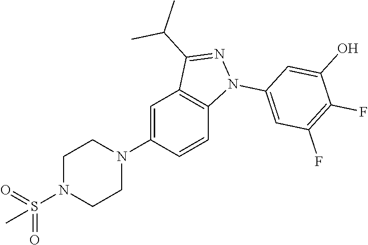 [SMILES: CC(C)c1nn(-c2cc(O)c(F)c(F)c2)c2ccc(N3CCN(S(C)(=O)=O)CC3)cc12] | 2,3-Difluoro-5-(3-isopropyl-5- (4-(methylsulfonyl)piperazin- 1-yl)-1H-indazol-1-yl)phenol | 451.2 |
| 21.04 | 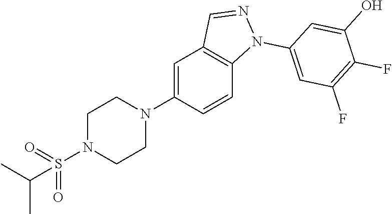 [SMILES: CC(C)S(=O)(=O)N1CCN(c2ccc3c(cnn3-c3cc(O)c(F)c(F)c3)c2)CC1] | 2,3-Difluoro-5-(5-(4-(isoprop- ylsulfonyl)piperazin-1-yl)-1H-indazol-1-yl)phenol | 437.1 |
| 21.05 (1) | 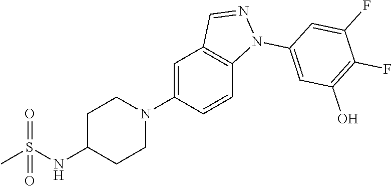 [SMILES: CS(=O)(=O)NC1CCN(c2ccc3c(cnn3-c3cc(O)c(F)c(F)c3)c2)CC1] | N-(1-(1-(3,4-Difluoro-5- hydroxyphenyl)-1H-indazol- 5-yl)piperidin-4-yl)methane- sulfonamide | 423.1 |
| 21.06 | 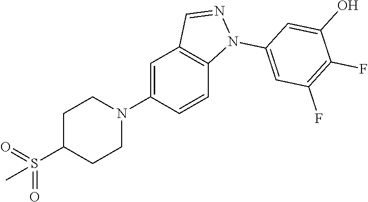 [SMILES: CS(=O)(=O)C1CCN(c2ccc3c(cnn3-c3cc(O)c(F)c(F)c3)c2)CC1] | 2,3-Difluoro-5-(5-(4-(methyl- sulfonyl)piperidin-1-yl)-1H- indazol-1-yl)phenol | 408.1 |
| 21.07 (1,2) | 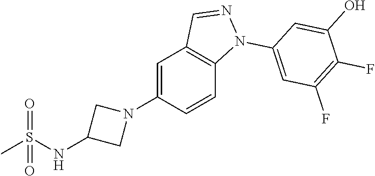 [SMILES: CS(=O)(=O)NC1CN(c2ccc3c(cnn3-c3cc(O)c(F)c(F)c3)c2)C1] | N-(1-(1-(3,4-Difluoro-5- hydroxyphenyl)-1H-indazol- 5-yl)azetidine-3-yl)methane- sulfonamide | 395.1 |
| 21.08 | 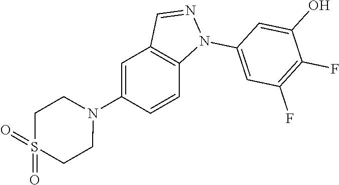 [SMILES: O=S1(=O)CCN(c2ccc3c(cnn3-c3cc(O)c(F)c(F)c3)c2)CC1] | 4-(1-(3,4-Difluoro-5-hydroxy- phenyl)-1H-indazol-5-yl)- thiomorpholine 1,1-dioxide | 380.1 |
| 21.09 | 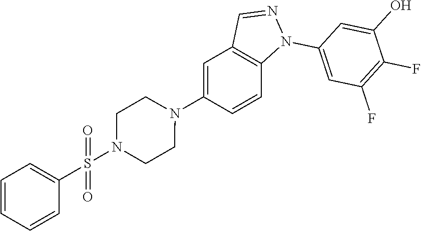 [SMILES: O=S(=O)(c1ccccc1)N1CCN(c2ccc3c(cnn3-c3cc(O)c(F)c(F)c3)c2)CC1] | 2,3-Difluoro-5-(5-(4-(phenyl- sulfonyl)piperazin-1-yl)-1H- indazol-1-yl)phenol | 471.1 |
| 21.10 | 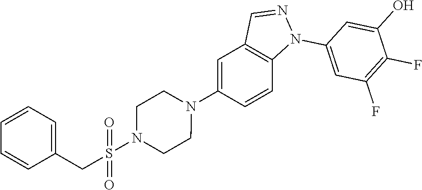 [SMILES: O=S(=O)(Cc1ccccc1)N1CCN(c2ccc3c(cnn3-c3cc(O)c(F)c(F)c3)c2)CC1] | 5-(5-(4-(Benzylsulfonyl)- piperazin-1-yl)-1H-indazol- 1-yl)-2,3-difluorophenol | 485.2 |
| 21.11 | 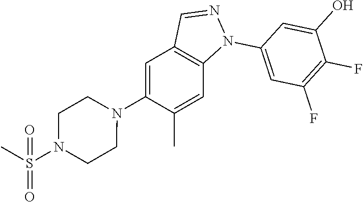 [SMILES: Cc1cc2c(cnn2-c2cc(O)c(F)c(F)c2)cc1N1CCN(S(C)(=O)=O)CC1] | 2,3-Difluoro-5-(6-methyl-5- (4-(methylsulfonyl)piperazin- 1-yl)-1H-indazol-1-yl)phenol | 423.1 |
| 21.12 | 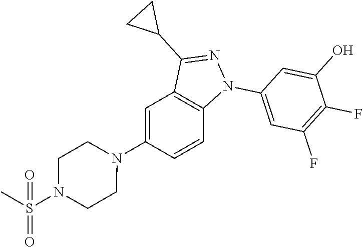 [SMILES: CS(=O)(=O)N1CCN(c2ccc3c(c2)c(C2CC2)nn3-c2cc(O)c(F)c(F)c2)CC1] | 5-(3-Cyclopropyl-5-(4-(meth- ylsulfonyl)piperazin-1-yl)-1H- indazol-1-yl)-2,3-difluoro- phenol | 449.1 |
| 21.13 (3) | 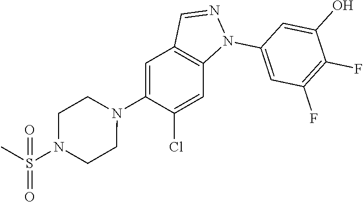 [SMILES: CS(=O)(=O)N1CCN(c2cc3cnn(-c4cc(O)c(F)c(F)c4)c3cc2Cl)CC1] | 5-(6-Chloro-5-(4-(methyl- sulfonyl)piperazin-1-yl)-1H- indazol-1-yl)-2,3-difluoro- phenol | 443.1 |
| 21.14 | 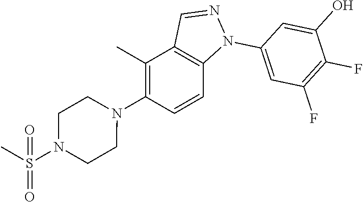 [SMILES: Cc1c(N2CCN(S(C)(=O)=O)CC2)ccc2c1cnn2-c1cc(O)c(F)c(F)c1] | 2,3-Difluoro-5-(4-methyl-5- (4-(methylsulfonyl)piperazin- 1-yl)-1H-indazol-1-yl)phenol | 423.1 |
| 21.15 (1) | 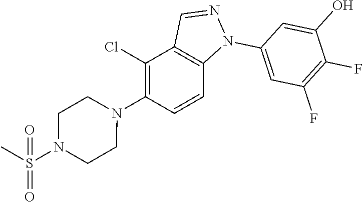 [SMILES: CS(=O)(=O)N1CCN(c2ccc3c(cnn3-c3cc(O)c(F)c(F)c3)c2Cl)CC1] | 5-(4-Chloro-5-(4-(methyl- sulfonyl)piperazin-1-yl)-1H- indazol-1-yl)-2,3-difluoro- phenol | 443.1 |
| 21.16 (3) | 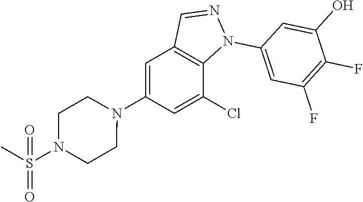 [SMILES: CS(=O)(=O)N1CCN(c2cc(Cl)c3c(cnn3-c3cc(O)c(F)c(F)c3)c2)CC1] | 5-(7-Chloro-5-(4-(methyl- sulfonyl)piperazin-1-yl)-1H- indazol-1-yl)-2,3-difluoro- phenol | 443.1 |
| 21.17 | 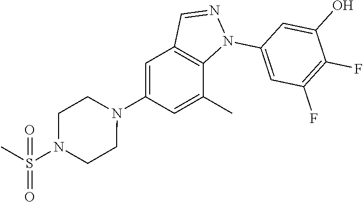 [SMILES: Cc1cc(N2CCN(S(C)(=O)=O)CC2)cc2cnn(-c3cc(O)c(F)c(F)c3)c12] | 2,3-Difluoro-5-(7-methyl-5- (4-(methylsulfonyl)piperazin- 1-yl)-1H-indazol-1-yl)phenol | 423.1 |
| 21.18 (3) | 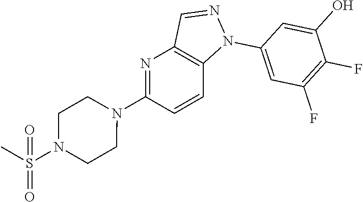 [SMILES: CS(=O)(=O)N1CCN(c2ccc3c(cnn3-c3cc(O)c(F)c(F)c3)n2)CC1] | 2,3-Difluoro-5-(5-(4-(methyl- sulfonyl)piperazin-1-yl)-1H- pyrazolo[4,3-b]pyridine-1-yl)- phenol | 410.1 |
| 21.19 | 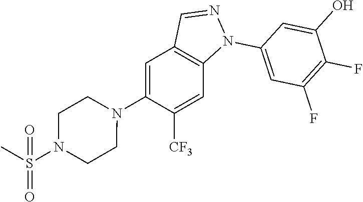 [SMILES: CS(=O)(=O)N1CCN(c2cc3cnn(-c4cc(O)c(F)c(F)c4)c3cc2C(F)(F)F)CC1] | 2,3-Difluoro-5-(5-(4-(methyl- sulfonyl)piperazin-1-yl)-6- (trifluoromethyl)-1H-indazol- 1-yl)phenol | 477.1 |
| 21.20 | 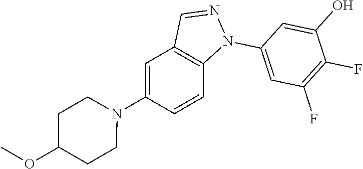 [SMILES: COC1CCN(c2ccc3c(cnn3-c3cc(O)c(F)c(F)c3)c2)CC1] | 2,3-Difluoro-5-(5-(4-methoxy- piperidin-1-yl)-1H-indazol-1- yl)phenol | 360.1 |
| 21.21 (3) | 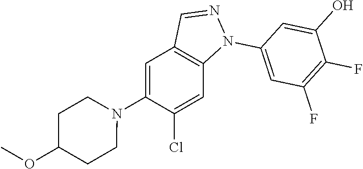 [SMILES: COC1CCN(c2cc3cnn(-c4cc(O)c(F)c(F)c4)c3cc2Cl)CC1] | 5-(6-Chloro-5-(4-methoxy- piperidin-1-yl)-1H-indazol-1- yl)-2,3-difluorophenol | 394.1 |
| 21.22 | 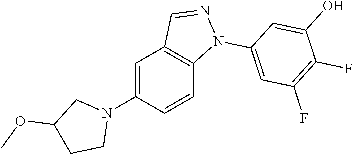 [SMILES: COC1CCN(c2ccc3c(cnn3-c3cc(O)c(F)c(F)c3)c2)C1] | 2,3-Difluoro-5-(5-(3-methoxy- pyrrolidin-1-yl)-1H-indazol-1- yl)phenol | 346.1 |
| 21.23 | 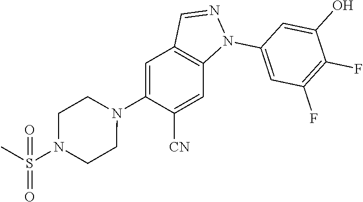 [SMILES: CS(=O)(=O)N1CCN(c2cc3cnn(-c4cc(O)c(F)c(F)c4)c3cc2C#N)CC1] | 1-(3,4-Difluoro-5-hydroxy- phenyl)-5-(4-(methylsulfonyl)- piperazin-1-yl)-1H-indazole-6- carbonitrile | 434.1 |
| 21.24 (1) | 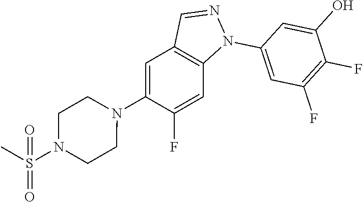 [SMILES: CS(=O)(=O)N1CCN(c2cc3cnn(-c4cc(O)c(F)c(F)c4)c3cc2F)CC1] | 2,3-Difluoro-5-(6-fluoro-5-(4- (methylsulfonyl)piperazin-1- yl)-1H-indazol-1-yl)phenol | 427.1 |
| 21.25 (2) | 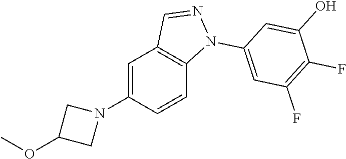 [SMILES: COC1CN(c2ccc3c(cnn3-c3cc(O)c(F)c(F)c3)c2)C1] | 2,3-Difluoro-5-(5-(3-methoxy- azetidin-1-yl)-1H-indazol-1- yl)phenol | 332.1 |
| 21.26 (2,4) | 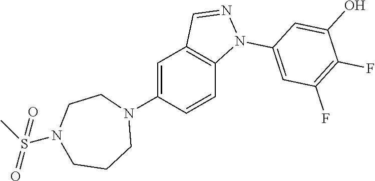 [SMILES: CS(=O)(=O)N1CCCN(c2ccc3c(cnn3-c3cc(O)c(F)c(F)c3)c2)CC1] | 2,3-Difluoro-5-(5-(4-(methyl- sulfonyl)-1,4-diazepan-1-yl)- 1H-indazol-1-yl)phenol | 423.1 |
| 21.27 (2,4) | 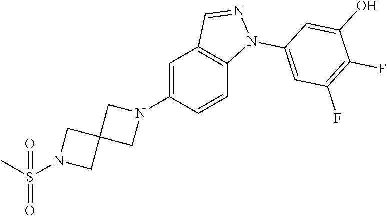 [SMILES: CS(=O)(=O)N1CC2(CN(c3ccc4c(cnn4-c4cc(O)c(F)c(F)c4)c3)C2)C1] | 2,3-Difluoro-5-(5-(6-(methyl- sulfonyl)-2,6-diazaspiro[3.3]- heptan-2-yl)-1H-indazol-1- yl)phenol | 421.1 |
| 21.28 (2,4) | 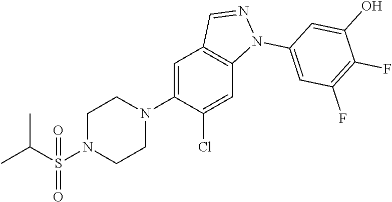 [SMILES: CC(C)S(=O)(=O)N1CCN(c2cc3cnn(-c4cc(O)c(F)c(F)c4)c3cc2Cl)CC1] | 5-(6-Chloro-5-(4-(isopropyl- sulfonyl)piperazin-1-yl)-1H- indazol-1-yl)-2,3-difluoro- phenol | 471.1 |
| 21.29 | 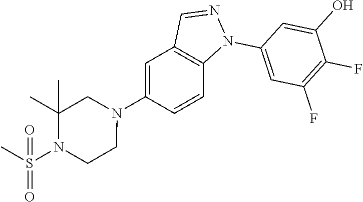 [SMILES: CC1(C)CN(c2ccc3c(cnn3-c3cc(O)c(F)c(F)c3)c2)CCN1S(C)(=O)=O] | 5-(5-(3,3-Dimethyl-4-(methyl- sulfonyl)piperazin-1-yl)-1H- indazol-1-yl)-2,3-difluoro- phenol | 437.2 |
| 21.30 | 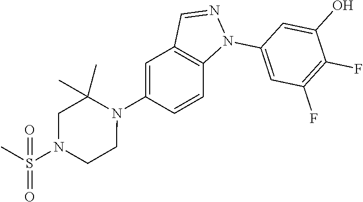 [SMILES: CC1(C)CN(S(C)(=O)=O)CCN1c1ccc2c(cnn2-c2cc(O)c(F)c(F)c2)c1] | 5-(5-(2,2-Dimethyl-4-(methyl- sulfonyl)piperazin-1-yl)-1H- indazol-1-yl)-2,3-difluoro- phenol | 437.1 |
| 21.31 (2) | 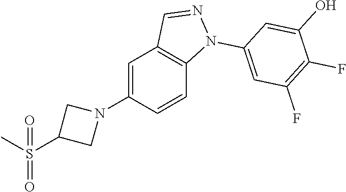 [SMILES: CS(=O)(=O)C1CN(c2ccc3c(cnn3-c3cc(O)c(F)c(F)c3)c2)C1] | 2,3-Difluoro-5-(5-(3-(methyl- sulfonyl)azetidin-1-yl)-1H- indazol-1-yl)phenol | 380.1 |
| 21.32 (2,4) | 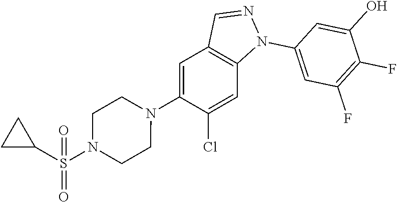 [SMILES: O=S(=O)(C1CC1)N1CCN(c2cc3cnn(-c4cc(O)c(F)c(F)c4)c3cc2Cl)CC1] | 5-(6-Chloro-5-(4-(cycloprop- ylsulfonyl)piperazin-1-yl)-1H- indazol-1-yl)-2,3-difluoro- phenol | 469.1 |
| 21.33 (2,4) | 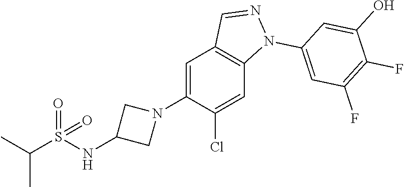 [SMILES: CC(C)S(=O)(=O)NC1CN(c2cc3cnn(-c4cc(O)c(F)c(F)c4)c3cc2Cl)C1] | N-(1-(6-Chloro-1-(3,4-difluoro- 5-hydroxyphenyl)-1H-indazol- 5-yl)azetidin-3-yl)propane-2- sulfonamide | 457.1 |
| 21.34 (2,4) | 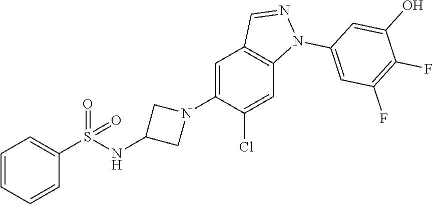 [SMILES: O=S(=O)(NC1CN(c2cc3cnn(-c4cc(O)c(F)c(F)c4)c3cc2Cl)C1)c1ccccc1] | N-(1-(6-Chloro-1-(3,4-difluoro- 5-hydroxyphenyl)-1H-indazol- 5-yl)azetidin-3-yl)benzene- sulfonamide | 491.1 |
| 21.35 (2) | 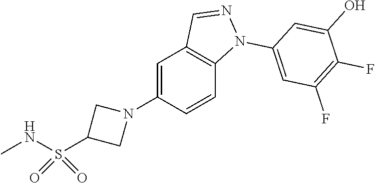 [SMILES: CNS(=O)(=O)C1CN(c2ccc3c(cnn3-c3cc(O)c(F)c(F)c3)c2)C1] | 1-(1-(3,4-Difluoro-5-hydroxy- phenyl)-1H-indazol-5-yl)-N- methylazetidine-3-sulfonamide | 395.1 |
| 21.36 (2) | 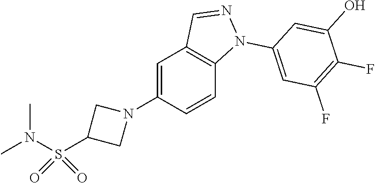 [SMILES: CN(C)S(=O)(=O)C1CN(c2ccc3c(cnn3-c3cc(O)c(F)c(F)c3)c2)C1] | 1-(1-(3,4-Difluoro-5-hydroxy- phenyl)-1H-indazol-5-yl)-N,N- dimethylazetidine-3-sulfon- amide | 409.2 |
| 21.37 (2,4) | 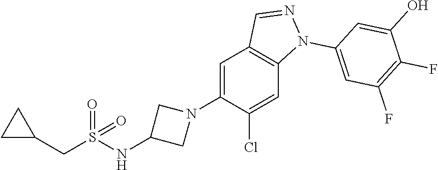 [SMILES: O=S(=O)(CC1CC1)NC1CN(c2cc3cnn(-c4cc(O)c(F)c(F)c4)c3cc2Cl)C1] | N-(1-(6-Chloro-1-(3,4-difluoro- 5-hydroxyphenyl)-1H-indazol- 5-yl)azetidin-3-yl)-1-cyclo- propylmethanesulfonamide | 469.0 |
| 21.38 | 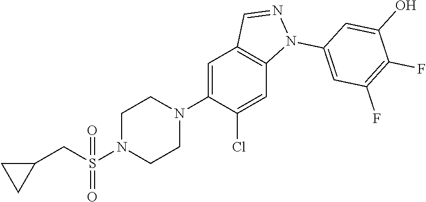 [SMILES: O=S(=O)(CC1CC1)N1CCN(c2cc3cnn(-c4cc(O)c(F)c(F)c4)c3cc2Cl)CC1] | 5-(6-Chloro-5-(4-((cycloprop- ylmethyl)sulfonyl)piperazin- 1-yl)-1H-indazol-1-yl)-2,3- difluorophenol | 483.0 |
| 21.39 | 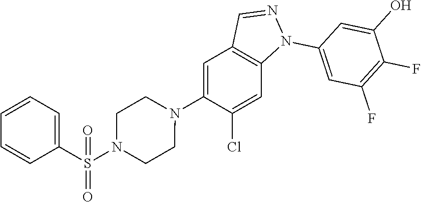 [SMILES: O=S(=O)(c1ccccc1)N1CCN(c2cc3cnn(-c4cc(O)c(F)c(F)c4)c3cc2Cl)CC1] | 5-(6-Chloro-5-(4-(phenyl- sulfonyl)piperazin-1-yl)-1H- indazol-1-yl)-2,3-difluoro- phenol | 505.0 |
| 21.40 | 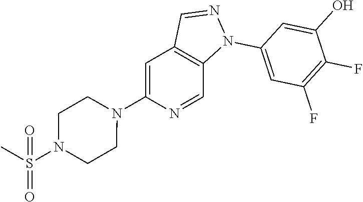 [SMILES: CS(=O)(=O)N1CCN(c2cc3cnn(-c4cc(O)c(F)c(F)c4)c3cn2)CC1] | 2,3-Difluoro-5-(5-(4-(methyl- sulfonyl)piperazin-1-yl)-1H- pyrazolo[3,4-c]pyridine-1-yl)- phenol | 410.1 |
| 21.41 (5,6) | 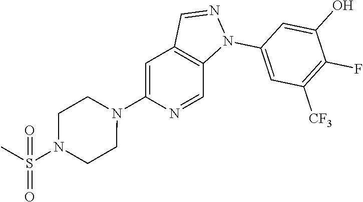 [SMILES: CS(=O)(=O)N1CCN(c2cc3cnn(-c4cc(O)c(F)c(C(F)(F)F)c4)c3cn2)CC1] | 2-Fluoro-5-(5-(4-(methyl- sulfonyl)piperazin-1-yl)-1H- pyrazolo[3,4-c]pyridine-1-yl)- 3-(trifluoromethyl)phenol | 460.0 |
| 21.42 (5,6) | 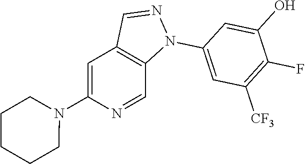 [SMILES: Oc1cc(-n2ncc3cc(N4CCCCC4)ncc32)cc(C(F)(F)F)c1F] | 2-Fluoro-5-(5-(piperidin-1-yl)- 1H-pyrazolo[3,4-c]pyridine-1- yl)-3-(trifluoromethyl)phenol | 381.1 |
| 21.43 (5,6) | 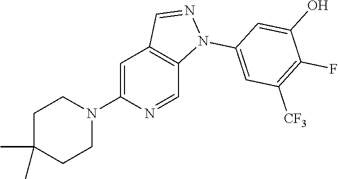 [SMILES: CC1(C)CCN(c2cc3cnn(-c4cc(O)c(F)c(C(F)(F)F)c4)c3cn2)CC1] | 5-(5-(4,4-Dimethylpiperidin- 1-yl)-1H-pyrazolo[3,4-c]- pyridine-1-yl)-2-fluoro-3- (trifluoromethyl)phenol | 409.2 |
| 21.44 (5,6) | 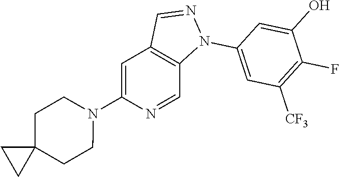 [SMILES: Oc1cc(-n2ncc3cc(N4CCC5(CC4)CC5)ncc32)cc(C(F)(F)F)c1F] | 5-(5-(6-Azaspiro[2.5]octan-6- yl)-1H-pyrazolo[3,4-c]pyridine- 1-yl)-2-fluoro-3-(trifluoro- methyl)phenol | 407.0 |
| 21.45 (1) | 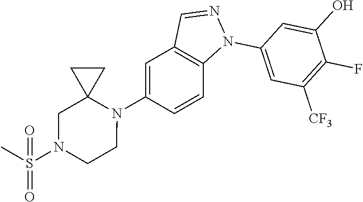 [SMILES: CS(=O)(=O)N1CCN(c2ccc3c(cnn3-c3cc(O)c(F)c(C(F)(F)F)c3)c2)C2(CC2)C1] | 2-Fluoro-5-(5-(7-(methyl- sulfonyl)-4,7-diazaspiro[2.5]- octan-4-yl)-1H-indazol-1-yl)- 3-(trifluoromethyl)phenol | 485.0 |
| 21.46 (1) | 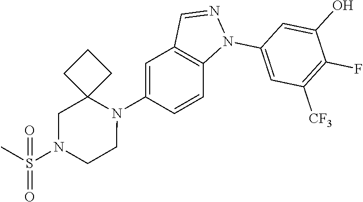 [SMILES: CS(=O)(=O)N1CCN(c2ccc3c(cnn3-c3cc(O)c(F)c(C(F)(F)F)c3)c2)C2(CCC2)C1] | 2-Fluoro-5-(5-(8-(methyl- sulfonyl)-5,8-diazaspiro[3.5]- nonan-5-yl)-1H-indazol-1-yl)- 3-(trifluoromethyl)phenol | 499.0 |
| 21.47 (1) | 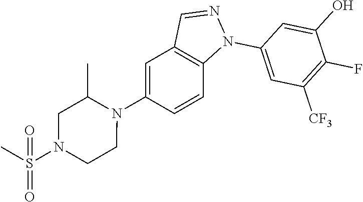 [SMILES: CC1CN(S(C)(=O)=O)CCN1c1ccc2c(cnn2-c2cc(O)c(F)c(C(F)(F)F)c2)c1] | 2-Fluoro-5-(5-(2-methyl-4- (methylsulfonyl)piperazin-1- yl)-1H-indazol-1-yl)-3- (trifluoromethyl)phenol | 473.0 |
| 21.48 (1) | 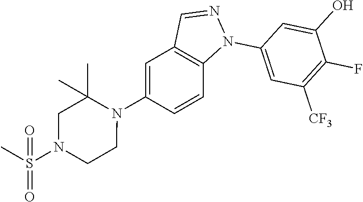 [SMILES: CC1(C)CN(S(C)(=O)=O)CCN1c1ccc2c(cnn2-c2cc(O)c(F)c(C(F)(F)F)c2)c1] | 5-(5-(2,2-Dimethyl-4-(methyl- sulfonyl)piperazin-1-yl)-1H- indazol-1-yl)-2-fluoro-3- (trifluoromethyl)phenol | 487.0 |
| 21.49 | 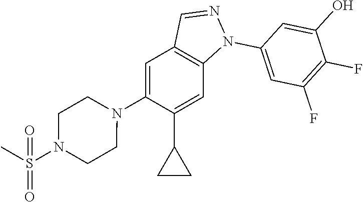 [SMILES: CS(=O)(=O)N1CCN(c2cc3cnn(-c4cc(O)c(F)c(F)c4)c3cc2C2CC2)CC1] | 5-(6-Cyclopropyl-5-(4-(meth- ylsulfonyl)piperazin-1-yl)- 1H-indazol-1-yl)-2,3-difluoro- phenol | 449.1 |
| 21.50 (7,2) | 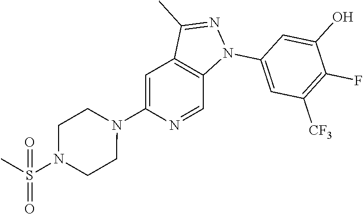 [SMILES: Cc1nn(-c2cc(O)c(F)c(C(F)(F)F)c2)c2cnc(N3CCN(S(C)(=O)=O)CC3)cc12] | 2-Fluoro-5-(3-methyl-5-(4- (methylsulfonyl)piperazin-1- yl)-1H-pyrazolo[3,4-c]- pyridine-1-yl)-3-(trifluoro- methyl)phenol | 474.0 |
| 21.51 (7,2) | 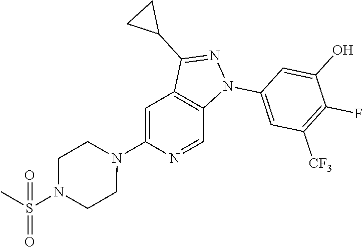 [SMILES: CS(=O)(=O)N1CCN(c2cc3c(C4CC4)nn(-c4cc(O)c(F)c(C(F)(F)F)c4)c3cn2)CC1] | 5-(3-Cyclopropyl-5-(4-(meth- ylsulfonyl)piperazin-1-yl)-1H- pyrazolo[3,4-c]pyridine-1-yl)- 2-fluoro-3-(trifluoromethyl)- phenol | 500.1 |
| 21.52 (7,2) | 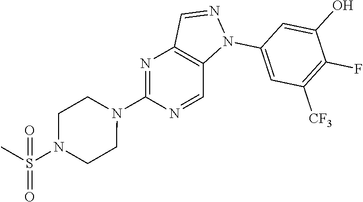 [SMILES: CS(=O)(=O)N1CCN(c2ncc3c(cnn3-c3cc(O)c(F)c(C(F)(F)F)c3)n2)CC1] | 2-Fluoro-5-(5-(4-(methyl- sulfonyl)piperazin-1-yl)-1H- pyrazolo[4,3-d]pyrimidin-1- yl)-3-(trifluoromethyl)phenol | 461.0 |
| 21.53 (7) | 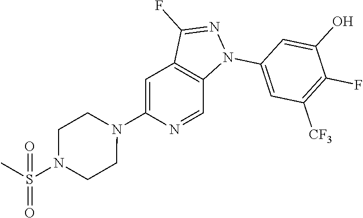 [SMILES: CS(=O)(=O)N1CCN(c2cc3c(F)nn(-c4cc(O)c(F)c(C(F)(F)F)c4)c3cn2)CC1] | 2-Fluoro-5-(3-fluoro-5-(4- (methylsulfonyl)piperazin-1- yl)-1H-pyrazolo[3,4-c]pyridin- 1-yl)-3-(trifluoromethyl)phenol | 478.0 |
| 21.54 (5,6) | 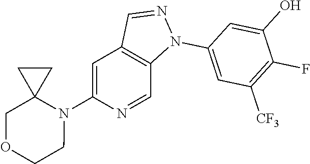 [SMILES: Oc1cc(-n2ncc3cc(N4CCOCC45CC5)ncc32)cc(C(F)(F)F)c1F] | 5-(5-(7-Oxa-4-azaspiro[2.5]- octan-4-yl)-1H-pyrazolo[3,4- c]pyridin-1-yl)-2-fluoro-3- (trifluoromethyl)phenol | 409.0 |
| 21.55 (5,6) | 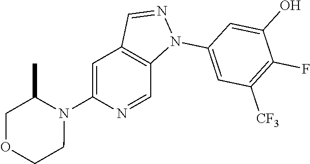 [SMILES: C[C@@H]1COCCN1c1cc2cnn(-c3cc(O)c(F)c(C(F)(F)F)c3)c2cn1] | (R)-2-Fluoro-5-(5-(3-methyl- morpholino)-1H-pyrazolo[3,4- c]pyridin-1-yl)-3-(trifluoro- methyl)phenol | 397.2 |
| 21.56 (5,6) | 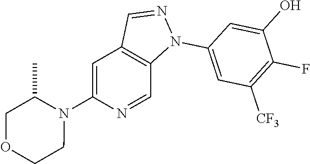 [SMILES: C[C@H]1COCCN1c1cc2cnn(-c3cc(O)c(F)c(C(F)(F)F)c3)c2cn1] | (S)-2-Fluoro-5-(5-(3-methyl- morpholino)-1H-pyrazolo[3,4- c]pyridin-1-yl)-3-(trifluoro- methyl)phenol | 397.1 |
| 21.57 (5,6) | 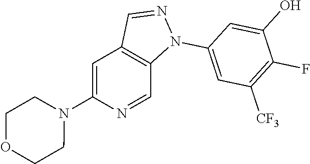 [SMILES: Oc1cc(-n2ncc3cc(N4CCOCC4)ncc32)cc(C(F)(F)F)c1F] | 2-Fluoro-5-(5-morpholino-1H- pyrazolo[3,4-c]pyridin-1-yl)- 3-(trifluoromethyl)phenol | 383.1 |
| 21.58 (5,6) | 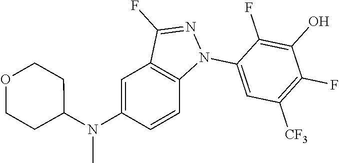 [SMILES: CN(c1ccc2c(c1)c(F)nn2-c1cc(C(F)(F)F)c(F)c(O)c1F)C1CCOCC1] | 2,6-Difluoro-3-(3-fluoro-5- (methyl(tetrahydro-2H-pyran-4- yl)amino)-1H-indazol-1-yl)-5- (trifluoromethyl)phenol | 446.3 |
| 21.59 (4,8) | 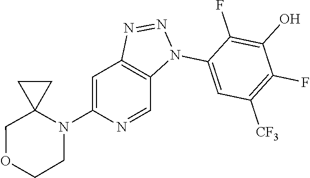 [SMILES: Oc1c(F)c(-n2nnc3cc(N4CCOCC45CC5)ncc32)cc(C(F)(F)F)c1F] | 3-(6-(7-Oxa-4-azaspiro[2.5]- octan-4-yl)-3H-[1,2,3]triazolo- [4,5-c]pyridin-3-yl)-2,6- difluoro-5-(trifluoromethyl)- phenol | 428.0 |
| 21.60 (6,10,11) | 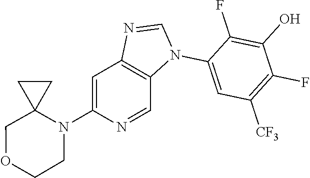 [SMILES: Oc1c(F)c(-n2cnc3cc(N4CCOCC45CC5)ncc32)cc(C(F)(F)F)c1F] | 3-(6-(7-Oxa-4-azaspiro[2.5]- octan-4-yl)-3H-imidazo[4,5- c]pyridin-3-yl)-2,6-difluoro-5- (trifluoromethyl)phenol | 427.2 |
| 21.61 (9) | 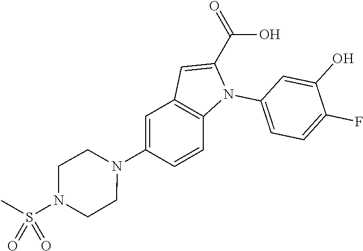 [SMILES: CS(=O)(=O)N1CCN(c2ccc3c(c2)cc(C(=O)O)n3-c2ccc(F)c(O)c2)CC1] | 1-(4-Fluoro-3-hydroxyphenyl)- 5-(4-(methylsulfonyl)piperazin- 1-yl)-1H-indole-2-carboxylic acid | 434.1 |
| 21.62 (9) | 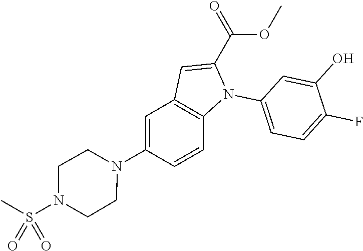 [SMILES: COC(=O)c1cc2cc(N3CCN(S(C)(=O)=O)CC3)ccc2n1-c1ccc(F)c(O)c1] | Methyl 1-(4-fluoro-3-hydroxy- phenyl)-5-(4-(methylsulfonyl)- piperazin-1-yl)-1H-indole-2- carboxylate | 448.2 |
| 21.63 (6,10,11) | 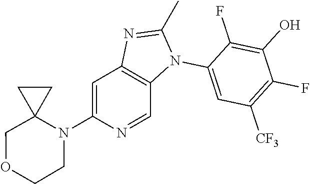 [SMILES: Cc1nc2cc(N3CCOCC34CC4)ncc2n1-c1cc(C(F)(F)F)c(F)c(O)c1F] | 2,6-Difluoro-3-(2-methyl-6-(7- oxa-4-azaspiro[2.5]octan-4-yl)- 3H-imidazo[4,5-c]pyridin-3-yl)- 5-(trifluoromethyl)phenol | 441.2 |

Alternate conditions used: 1. Step 1: NaO^{t}Bu, Pd(OAc)₂, P Bu₃, toluene, 100° C.; 2. Step 2: TFA, DCM, rt; 3. Step 1: Cs₂CO₃, BINAP, Pd₂(dba)₃, toluene, 100° C.; 4. Step 1: RuPhos Pd G3, NaO^{t}Bu, toluene or dioxane, 100° C., 12 h; 5. No phenol protecting group; 6. Toluene was replaced with dioxane; 7. Step 1: NaO^{t}Bu, Pd(OAc)₂, XPhos, toluene, 100° C.; 8. Step 2: Pd/C, THF, H₂, rt, 2 h; 9. Step 2: 1 M LiOH:THF:CH₃OH (1:1:1), rt, 15 h; 10. NaOtBu was replaced with Cs₂CO₃; 11. Step 2: TFA, 70° C., 2 h.

### Compound 22

### 5-(5-((3-Chloro-4-methoxyphenyl)amino)-1H-indazol-1-yl)-2-fluorophenol

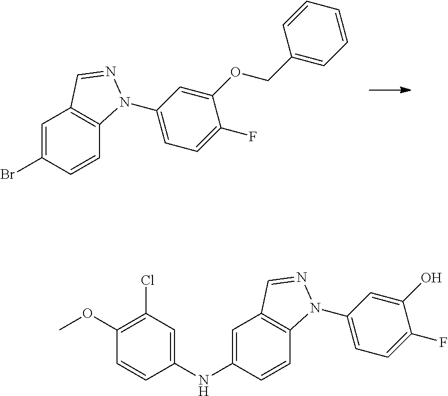 [SMILES: Fc1ccc(-n2ncc3cc(Br)ccc32)cc1OCc1ccccc1 ; COc1ccc(Nc2ccc3c(cnn3-c3ccc(F)c(O)c3)c2)cc1Cl]

A mixture of Intermediate 14.08 (0.25 g, 0.63 mmol), 3-chloro-4-methoxyphenylamine (0.20 g, 1.26 mmol), Pd₂(dba)₃ (0.030 g, 0.033 mmol), BINAP (0.041 g, 0.066 mmol), toluene (3 mL), and NaO^{t}Bu (0.96 mL, 1.93 mmol) was heated at 110° C. for 90 min, allowed to cool to room temperature, diluted (20 mL EtOAc), washed (20 mL water and then 20 mL brine), dried (Na₂SO₄), and then concentrated. The residue was purified by silica gel chromatography (0-20% EtOAc/hexanes) to give 1-(3-(benzyloxy)-4-fluorophenyl)-N-(3-chloro-4-methoxyphenyl)-1H-indazol-5-amine as an orange gum. The intermediate was dissolved in THF (5 mL). Pd/C (10%, 0.035 g) was added. The reaction was stirred under a balloon of hydrogen for 1 h and filtered through a Celite plug. The filter cake was rinsed with 10 mL THF. The filtrate was concentrated and purified by silica gel chromatography (0-20% EtOAc/hexanes) to give 5-(5-((3-chloro-4-methoxyphenyl)amino)-1H-indazol-1-yl)-2-fluorophenol (18 mg, 73%) as a beige foam. ¹H NMR (400 MHz, DMSO-d₆): δ 10.34 (s, 1H), 8.20-8.17 (m, 1H), 8.05 (s, 1H), 7.72 (d, J=9.0 Hz, 1H), 7.41-7.30 (m, 3H), 7.21 (dd, J 2.1, 9.0 Hz, 1H), 7.18-7.13 (m, 1H), 7.12-7.00 (m, 3H), 3.80 (s, 3H); LCMS 384.0 [M+H]⁺.

### Compound 23

### 2-Chloro-4-((1-(4-fluoro-3-hydroxyphenyl)-1H-indazol-5-yl)amino)phenol

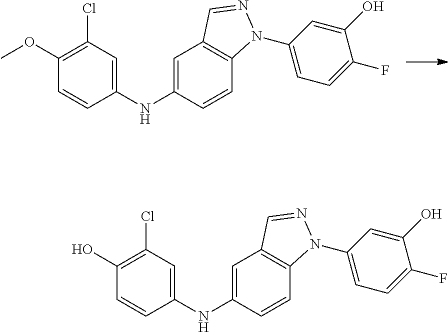 [SMILES: COc1ccc(Nc2ccc3c(cnn3-c3ccc(F)c(O)c3)c2)cc1Cl ; Oc1cc(-n2ncc3cc(Nc4ccc(O)c(Cl)c4)ccc32)ccc1F]

2-Chloro-4-((1-(4-fluoro-3-hydroxyphenyl)-1H-indazol-5-yl)amino)phenol was synthesized from Compound 22 in a similar manner to that described for Compound 4, Step 2. Note: Boron tribromide was added at 0° C. ¹H NMR (400 MHz, DMSO-d₆): δ 10.32 (br s, 1H), 9.58 (br s, 1H), 8.15 (s, 1H), 8.08-7.72 (m, 1H), 7.69 (d, J=9.0 Hz, 1H), 7.35-7.29 (m, 3H), 7.19-7.13 (m, 2H), 7.04 (d, J=2.3 Hz, 1H), 6.96-6.86 (m, 2H); LCMS 370.0 [M+H]⁺.

### Compound 24

### 1-(4-((1-(3,4-Difluoro-5-hydroxyphenyl)-1H-indazol-5-yl)oxy)piperidin-1-yl)ethan-1-one

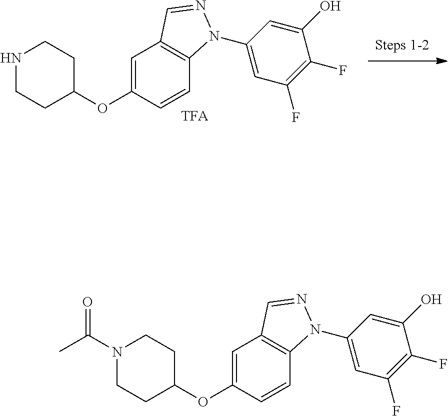 [SMILES: Oc1cc(-n2ncc3cc(OC4CCNCC4)ccc32)cc(F)c1F ; CC(=O)N1CCC(Oc2ccc3c(cnn3-c3cc(O)c(F)c(F)c3)c2)CC1]

### Step 1: 5-(5-((1-Acetylpiperidin-4-yl)oxy)-1H-indazol-1-yl)-2,3-difluorophenyl acetate

Acetic anhydride (29 mg, 0.29 mmol) was added to a solution of Intermediate 28.01 (110 mg, 0.32 mmol), Et₃N (133 μL, 0.96 mmol), and DCM (1 mL) at 0° C. The mixture was stirred at 20° C. for 1 h, quenched by addition of saturated NaHCO₃ (5 mL), and then extracted (3×10 mL DCM). The combined organic layers were washed (20 mL brine), dried (Na₂SO₄), filtered, and then concentrated to give 5-(5-((1-acetylpiperidin-4-yl)oxy)-1H-indazol-1-yl)-2,3-difluorophenyl acetate (200 mg) as a yellow solid. LCMS: 430.2 [M+H]⁺.

### Step 2: 1-(4-((1-(3,4-Difluoro-5-hydroxyphenyl)-1H-indazol-5-yl)oxy)piperidin-1-yl)ethan-1-one

LiOH—H₂O (59 mg, 1.40 mmol) was added to a mixture of 5-(5-((1-acetylpiperidin-4-yl)oxy)-1H-indazol-1-yl)-2,3-difluorophenyl acetate (200 mg, 0.47 mmol), THF (1 mL), and H₂O (0.3 mL). The mixture was stirred at room temperature for 2 h, concentrated, and then purified by prep-HPLC [water (0.04% HCl)-ACN] to give 1-(4-((1-(3,4-difluoro-5-hydroxyphenyl)-1H-indazol-5-yl)oxy)piperidin-1-yl)ethan-1-one (28 mg, 15%) as a white solid. Note: prior to prep-HPLC purification, the pH of the sample was adjusted to pH-3 with 1 M HCl. ¹H NMR (400 MHz, DMSO-d₆) δ 10.89 (s, 1H), 8.24 (s, 1H), 7.77 (d, 1H), 7.43 (d, 1H), 7.29-7.14 (m, 3H), 4.67-4.64 (m, 1H), 3.90-3.82 (m, 1H), 3.70 (d, 1H), 3.33 (s, 1H), 3.29-3.21 (m, 1H), 2.02 (s, 5H), 1.70-1.60 (m, 1H), 1.59-1.49 (m, 1H); LCMS: 388.1 [M+H]⁺.

The Compound below was synthesized in a similar manner to that described for Compound 24.

| Cmpd | Structure | Name | [M + H]⁺ |
|---|---|---|---|
| 24.01 | 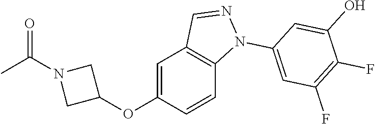 [SMILES: CC(=O)N1CC(Oc2ccc3c(cnn3-c3cc(O)c(F)c(F)c3)c2)C1] | 1-(3-((1-(3,4-Difluoro-5- hydroxyphenyl)-1H- indazol-5-yl)oxy)azetidin- 1-yl)ethan-1-one | 360.1 |

### Compound 25

### 2,3-Difluoro-5-(5-((1-(methylsulfonyl)azetidin-3-yl)oxy)-1H-indazol-1-yl)phenol

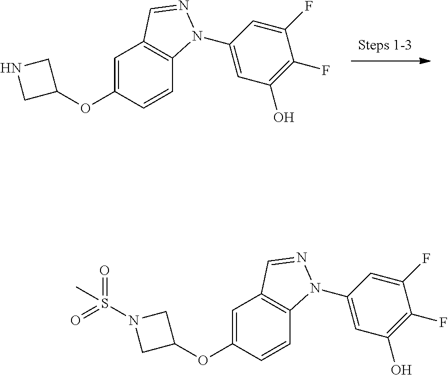 [SMILES: Oc1cc(-n2ncc3cc(OC4CNC4)ccc32)cc(F)c1F ; CS(=O)(=O)N1CC(Oc2ccc3c(cnn3-c3cc(O)c(F)c(F)c3)c2)C1]

### Step 1: 5-(Azetidin-3-yloxy)-1-(3-((tert-butyldimethylsilyl)oxy)-4,5-difluorophenyl)-1H-indazole

Imidazole (64 mg, 0.94 mmol) and DIPEA (367 mg, 2.84 mmol) were added to a solution of Intermediate 28 (free base, 300 mg, 0.94 mmol) and TBSCl (214 mg, 1.42 mmol) in DCM (8 mL). The mixture was stirred at room temperature for 1 h, poured into water (30 mL), and then extracted (3×35 mL EtOAc). The combined organic layers were washed (2×30 mL brine), dried (Na₂SO₄), filtered, and then concentrated to give 5-(azetidin-3-yloxy)-1-(3-((tert-butyldimethylsilyl)oxy)-4,5-difluorophenyl)-1H-indazole (280 mg) as a yellow oil. LCMS: 432.2 [M+H]⁺.

### Step 2: 1-(3-((tert-Butyldimethylsilyl)oxy)-4,5-difluorophenyl)-5-((1-(methylsulfonyl) azetidin-3-yl)oxy)-1H-indazole

Methanesulfonyl chloride (83 mg, 0.72 mmol) was added to a solution of 5-(azetidin-3-yloxy)-1-(3-((tert-butyldimethylsilyl)oxy)-4,5-difluorophenyl)-1H-indazole (260 mg, 0.60 mmol) and Et₃N (183 mg, 1.81 mmol) in DCM (6 mL) at 0° C. The mixture was stirred at room temperature for 1 h, poured into water (30 mL), and then extracted (3×3 5 mL EtOAc). The combined organic layers were washed (2×3 0 mL brine), dried (Na₂SO₄), filtered, and then concentrated to give 1-(3-((tert-butyldimethylsilyl)oxy)-4,5-difluorophenyl)-5-((1-(methylsulfonyl)azetidin-3-yl)oxy)-1H-indazole (240 mg) as a yellow oil. LCMS: 510.2 [M+H]⁺.

### Step 3: 2,3-Difluoro-5-(5-((1-(methylsulfonyl)azetidin-3-yl)oxy)-1H-indazol-1-yl)phenol

Lithium hydroxide monohydrate (59 mg, 1.41 mmol) was added to a solution of 1-(3-((tert-butyldimethylsilyl)oxy)-4,5-difluorophenyl)-5-((1-(methylsulfonyl)azetidin-3-yl)oxy)-1H-indazole (240 mg, 0.47 mmol), THF (4 mL), H₂O (2 mL), and MeOH (1 mL). The mixture was stirred at room temperature for 2 h. 1 M HCl was added to the mixture to adjust the pH to ˜7. The mixture was extracted (3×3 5 mL EtOAc), washed (2×30 mL brine), dried (Na₂SO₄), filtered, and then concentrated. The residue was purified by prep-HPLC [water (0.1% TFA)-ACN] to give 2,3-difluoro-5-(5-((1-(methylsulfonyl)azetidin-3-yl)oxy)-1H-indazol-1-yl)phenol (143 mg, 76%) as a white solid. ¹H NMR (400 MHz, DMSO-d₆): δ 11.30 (s, 1H), 8.24 (s, 1H), 7.81 (d, 1H), 7.37 (d, 1H), 7.37-7.14 (m, 3H), 5.13-5.07 (m, 1H), 4.36 (d, 2H), 3.96 (d, 2H), 3.08 (s, 3H); LCMS: 396.0 [M+H]⁺.

The Compound below was synthesized from Intermediate 28.01 using the following sequence: Compound 25, Step 2 (1 eq of MsCl, TEA, DCM, 0° C.-rt, 0.5 h), Compound 25, Step 2 (2 eq of MsCl, TEA, DCM, 0° C.-rt, 0.5 h), and then Compound 25, Step 3 (LiOH H₂O, THF/H₂O (3:1), rt, 2 h).

| Cmpd | Structure | Name | [M + H]⁺ |
|---|---|---|---|
| 25.01 | 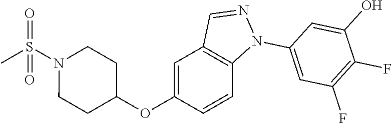 [SMILES: CS(=O)(=O)N1CCC(Oc2ccc3c(cnn3-c3cc(O)c(F)c(F)c3)c2)CC1] | 2,3-Difluoro-5-(5-((1-(methyl- sulfonyl)piperidin-4-yl)oxy)- 1H-indazol-1-yl)phenol | 424.0 |

### Compound 26

### 1-(4-(1-(4-Fluoro-3-hydroxyphenyl)-1H-indazol-5-yl)piperidin-1-yl)ethan-1-one

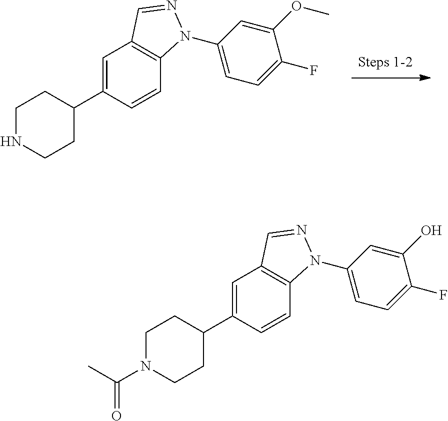

### Step 1: 1-(4-(1-(4-Fluoro-3-methoxyphenyl)-1H-indazol-5-yl)piperidin-1-yl)ethan-1-one

Acetyl chloride (160 μL, 2.21 mmol) was added to a mixture of Intermediate 26 (400 mg, 1.11 mmol), pyridine (900 μL, 11.1 mmol), and DCM (2 mL) at room temperature. The mixture was stirred for 2 h, poured into saturated NaHCO₃ (50 mL), and then extracted (3×50 mL EtOAc). The combined organic layers were washed (100 mL brine), dried (Na₂SO₄), filtered, and then concentrated. The residue was purified silica gel chromatography (50% EtOAc/petroleum ether) to give 1-(4-(1-(4-fluoro-3-methoxyphenyl)-1H-indazol-5-yl)piperidin-1-yl)ethan-1-one (250 mg, 61%) as a yellow oil. ¹H NMR (400 MHz, DMSO-d₆): δ 8.30 (s, 1H), 7.78 (d, 1H), 7.71 (s, 1H), 7.48 (dd, 1H), 7.45-7.37 (m, 2H), 7.32-7.26 (m, 1H), 4.56 (d, 1H), 3.99-3.90 (m, 4H), 3.16 (t, 1H), 2.99-2.85 (m, 1H), 2.69-2.56 (m, 1H), 2.04 (s, 3H), 1.85 (t, 2H), 1.74-1.59 (m, 1H), 1.57-1.43 (m, 1H); LCMS: 368.2 [M+H]⁺.

### Step 2: 1-(4-(1-(4-Fluoro-3-hydroxyphenyl)-1H-indazol-5-yl)piperidin-1-yl)ethan-1-one

Boron tribromide (330 μL, 3.40 mmol) was slowly added to a mixture of 1-(4-(1-(4-fluoro-3-methoxyphenyl)-1H-indazol-5-yl)piperidin-1-yl)ethan-1-one (250 mg, 0.68 mmol) in DCM (5 mL) at −78° C. via syringe. The mixture was warmed to room temperature, stirred for 2 h, slowly quenched with MeOH (10 mL), stirred for 0.5 h, diluted with saturated NaHCO₃ (20 mL), and then extracted (3×20 mL EtOAc). The combined organic layers were washed (20 mL brine), dried (Na₂SO₄), filtered, and then concentrated. The residue was purified by silica gel chromatography (40% EtOAc/petroleum ether) to give 1-(4-(1-(4-fluoro-3-hydroxyphenyl)-1H-indazol-5-yl)piperidin-1-yl)ethan-1-one (14 mg, 57%) as a white solid. ¹H NMR (400 MHz, DMSO-d₆): δ 10.32 (s, 1H), 8.27 (s, 1H), 7.75-7.67 (m, 2H), 7.42 (d, 1H), 7.37-7.28 (m, 2H), 7.20-7.10 (m, 1H), 4.56 (d, 1H), 3.94 (d, 1H), 3.16 (t, 1H), 2.97-2.84 (m, 1H), 2.66-2.56 (m, 1H), 2.04 (s, 3H), 1.91-1.77 (m, 2H), 1.66 (dq, 1H), 1.50 (dq, 1H); LCMS: 352.1 [M−H]⁻.

The Compounds below were synthesized in a similar manner to that described for Compound 26.

| Cmpd | Structure | Name | [M + H]⁺ |
|---|---|---|---|
| 26.01 | 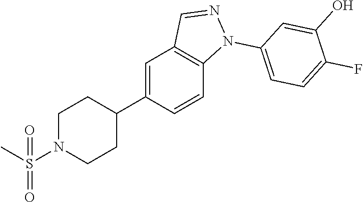 [SMILES: CS(=O)(=O)N1CCC(c2ccc3c(cnn3-c3ccc(F)c(O)c3)c2)CC1] | 2-Fluoro-5-(5-(1-(methyl- sulfonyl)piperidin-4-yl)- 1H-indazol-1-yl)phenol | 390.0 |
| 26.02 | 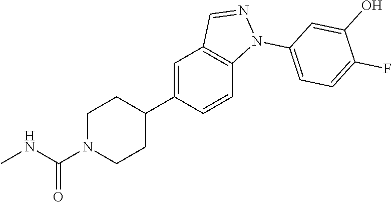 [SMILES: CNC(=O)N1CCC(c2ccc3c(cnn3-c3ccc(F)c(O)c3)c2)CC1] | 4-(1-(4-Fluoro-3-hydroxy- phenyl)-1H-indazol-5-yl)- N-methylpiperidine-1- carboxamide | 369.1 |

### Compound 27

### 2,3-Difluoro-5-(5-(1-(methylsulfonyl)piperidin-4-yl)-1H-indazol-1-yl)phenol

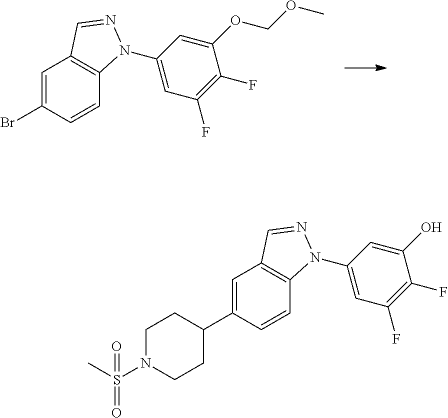

2,3-Difluoro-5-(5-(1-(methylsulfonyl)piperidin-4-yl)-1H-indazol-1-yl)phenol was synthesized from Intermediate 18 and 1-(methylsulfonyl)-4-(4,4,5,5-tetramethyl-1,3,2-dioxaborolan-2-yl)-1,2,3,6-tetrahydropyridine using the following sequence: Compound 21, Step 1 (Pd(dppf)Cl₂·CH₂Cl₂, 3 M K₃PO₄, THF, 80° C., 4 h), Intermediate 26 (Step 2), and then deprotection (TFA, DCM, rt, 1 h). ¹H NMR (400 MHz, DMSO-d₆): δ 10.89 (s, 1H), 8.32 (s, 1H), 7.80 (d, 1H), 7.75 (s, 1H), 7.47 (d, 1H), 7.30-7.10 (m, 2H), 3.71 (d, 2H), 2.92 (s, 3H), 2.90-2.70 (m, 3H), 1.93 (d, 2H), 1.80-1.60 (m, 2H); LCMS: 408.1 [M+H]⁺.

The Compound below was synthesized from Intermediate 18.05 and 1-(methylsulfonyl)-4-(4,4,5,5-tetramethyl-1,3,2-dioxaborolan-2-yl)-1,2,3,6-tetrahydropyridine using the following sequence: Compound 21, Step 1 (Pd(dppf)Cl₂, K₃PO₄, THF, H₂O, 70° C., overnight), hydrogenation (PtO₂, EtOAc, H₂, rt, 2 h), and then deprotection (TFA, DCM, rt, 0.5 h).

| Cmpd | Structure | Name | [M + H]⁺ |
|---|---|---|---|
| 27.01 | 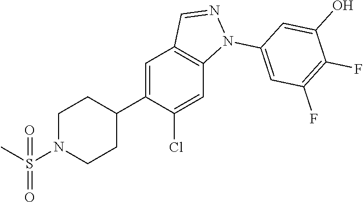 [SMILES: CS(=O)(=O)N1CCC(c2cc3cnn(-c4cc(O)c(F)c(F)c4)c3cc2Cl)CC1] | 5-(6-Chloro-5-(1-(methyl- sulfonyl)piperidin-4-yl)-1H- indazol-1-yl)-2,3-difluoro- phenol | 442.0 |

### Compound 28

### 4-(1-(3,4-Difluoro-5-hydroxyphenyl)-1H-indazol-5-yl)-1-(methylsulfonyl)piperidine-4-carboxylic Acid

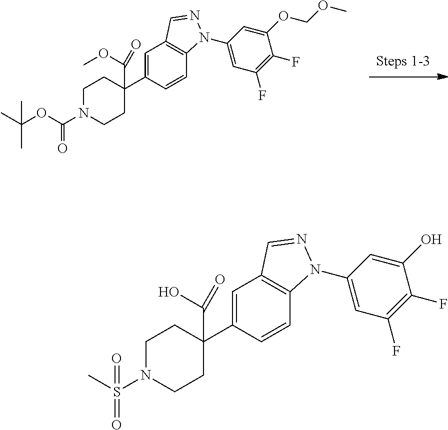

### Step 1: Methyl-4-(1-(3,4-difluoro-5-hydroxyphenyl)-1H-indazol-5-yl)piperidine-4-carboxylate TFA salt

A solution of Intermediate 29 (500 mg, 0.94 mmol) in TFA (4.2 mL, 56.4 mmol) and DCM (10 mL) was stirred at room temperature for 2 h then concentrated to give methyl-4-(1-(3,4-difluoro-5-hydroxyphenyl)-1H-indazol-5-yl)piperidine-4-carboxylate TFA salt (470 mg) as a yellow oil. LCMS: 388.1 [M+H]⁺.

### Step 2: Methyl-4-(1-(3,4-difluoro-5-((methylsulfonyl)oxy)phenyl)-1H-indazol-5-yl)-1-(methylsulfonyl)piperidine-4-carboxylate

Methanesulfonyl chloride (220 μL, 2.81 mmol) was added to a mixture of methyl-4-(1-(3,4-difluoro-5-hydroxyphenyl)-1H-indazol-5-yl)piperidine-4-carboxylate TFA salt (470 mg, 0.94 mmol), triethylamine (1.3 mL, 9.37 mmol), and DCM (5 mL) at 0° C. The mixture was stirred at room temperature for 2 h, poured into H₂O (50 mL), and then extracted (3×50 mL EtOAc). The combined organic layers were washed (50 mL brine), dried (Na₂SO₄), filtered, and then concentrated. The residue was purified by silica gel chromatography (30% EtOAc/petroleum ether) to give methyl-4-(1-(3,4-difluoro-5-((methylsulfonyl)oxy)phenyl)-1H-indazol-5-yl)-1-(methylsulfonyl)piperidine-4-carboxylate (250 mg, 49%) as a yellow oil. ¹H NMR (400 MHz, DMSO-d₆): δ 8.46 (d, 1H), 8.02-7.87 (m, 3H), 7.84-7.78 (m, 1H), 7.58 (dd, 1H), 3.65-3.60 (m, 6H), 3.57-3.48 (m, 2H), 2.97-2.85 (m, 5H), 2.71-2.57 (m, 2H), 2.10-2.00 (m, 2H); LCMS: 544.1 [M+H]⁺.

### Step 3: 4-(1-(3,4-Difluoro-5-hydroxyphenyl)-1H-indazol-5-yl)-1-(methylsulfonyl)piperidine-4-carboxylic Acid

A mixture of methyl-4-(1-(3,4-difluoro-5-((methyl sulfonyl)oxy)phenyl)-H-indazol-5-yl)-1-(methylsulfonyl)piperidine-4-carboxylate (250 mg, 0.46 mmol), LiOH·H₂O (193 mg, 4.60 mmol), THE (10 mL), MeOH (5 mL), and H₂O (5 mL) was heated at 50° C. for 4 h. HCl (1 M) was added to the reaction mixture to adjust to pH-5. The mixture was poured into H₂O (50 mL) and then extracted (3×50 mL EtOAc). The combined organic layers were washed (50 mL brine), dried (Na₂SO₄), filtered, and then concentrated. The residue was purified by prep-HPLC [water (0.04%2HCl)/CH₃CN] to give 4-(1-(3,4-difluoro-5-hydroxyphenyl)-1H-indazol-5-yl)-1-(methyl sulfonyl)piperidine-4-carboxylic acid (65 mg, 31%) as a white solid. ¹H NMR (400 MHz, DMSO-d₆): δ 12.84 (s, 1H), 10.94 (s, 1H), 8.38 (s, 1H), 7.91 (s, 1H), 7.85 (d, 1H), 7.59 (d, 1H), 7.34-7.24 (m, 1H), 7.21 (d, 1H), 3.53 (d, 2H), 2.98-2.82 (m, 5H), 2.61 (d, 2H), 1.97 (t, 2H); LCMS: 452.0 [M+H]⁺.

The Compounds below were synthesized from the appropriate Intermediate in a similar manner to that described for Compound 28.

| Cmpd | Structure | Name | [M + H]⁺ |
|---|---|---|---|
| 28.01 (1) | 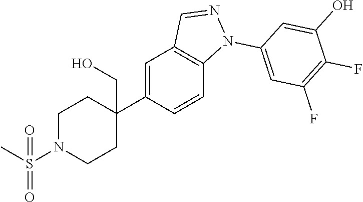 [SMILES: CS(=O)(=O)N1CCC(CO)(c2ccc3c(cnn3-c3cc(O)c(F)c(F)c3)c2)CC1] | 2,3-Difluoro-5-(5-(4-hydroxy- methyl)-1-(methylsulfonyl)- piperidin-4-yl)-1H-indazol-1- yl)phenol | 438.1 |
| 28.02 (1) | 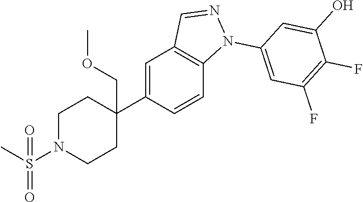 [SMILES: COCC1(c2ccc3c(cnn3-c3cc(O)c(F)c(F)c3)c2)CCN(S(C)(=O)=O)CC1] | 2,3-Difluoro-5-(5-(4-(meth- oxymethyl)-1-(methylsul- fonyl)piperidin-4-yl)-1H- indazol-1-yl)phenol | 452.0 |
| 28.03 | 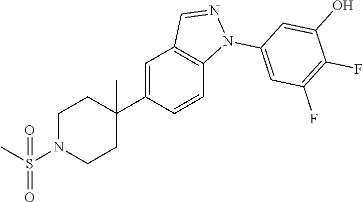 [SMILES: CC1(c2ccc3c(cnn3-c3cc(O)c(F)c(F)c3)c2)CCN(S(C)(=O)=O)CC1] | 2,3-Difluoro-5-(5-(4-methyl- 1-(methylsulfonyl)piperidin- 4-yl)-1H-indazol-1-yl)phenol | 422.0 |
| 28.04 | 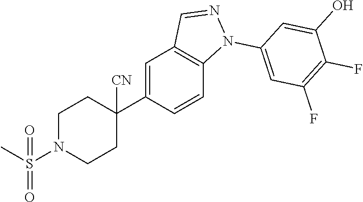 [SMILES: CS(=O)(=O)N1CCC(C#N)(c2ccc3c(cnn3-c3cc(O)c(F)c(F)c3)c2)CC1] | 4-(1-(3,4-Difluoro-5-hydroxy- phenyl)-1H-indazol-5-yl)-1- (methylsulfonyl)piperidine- 4-carbonitrile | 433.0 |
| 28.05 | 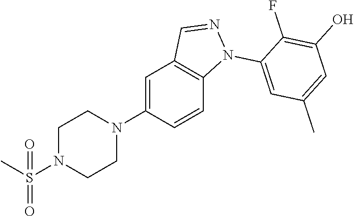 [SMILES: Cc1cc(O)c(F)c(-n2ncc3cc(N4CCN(S(C)(=O)=O)CC4)ccc32)c1] | 2-Fluoro-5-methyl-3-(5-(4- (methylsulfonyl)piperazin-1- yl)-1H-indazol-1-yl)phenol | 405.0 |
| 28.06 | 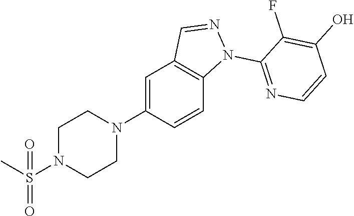 [SMILES: CS(=O)(=O)N1CCN(c2ccc3c(cnn3-c3nccc(O)c3F)c2)CC1] | 3-Fluoro-2-(5-(4-(methyl- sulfonyl)piperazin-1-yl)-1H- indazol-1-yl)pyridin-4-ol | 392.0 |
| 28.07 | 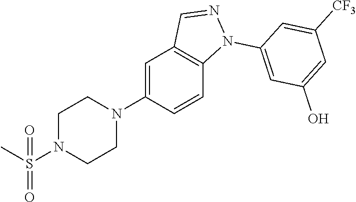 [SMILES: CS(=O)(=O)N1CCN(c2ccc3c(cnn3-c3cc(O)cc(C(F)(F)F)c3)c2)CC1] | 3-(5-(4-(Methylsulfonyl)- piperazin-1-yl)-1H-indazol-1- yl)-5-(trifluoromethyl)phenol | 441.0 |
| 28.08 (2) | 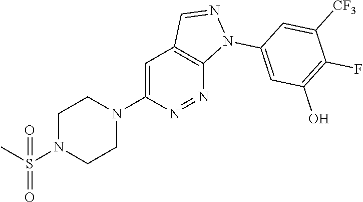 [SMILES: CS(=O)(=O)N1CCN(c2cc3cnn(-c4cc(O)c(F)c(C(F)(F)F)c4)c3nn2)CC1] | 2-Fluoro-5-(5-(4-(methyl- sulfonyl)piperazin-1-yl)-1H- pyrazolo[3,4-c]pyridazin-1- yl)-3-(trifluoromethyl)phenol | 461.0 |

Alternate conditions used: Step 1: 2-4 h. Step 2: 2 h-overnight. Step 3: rt-50° C., 1 h-overnight. 1. Step 1: 4 M HCl in EtOAc, rt, 0.5-1.5 h; 2. Steps 2 and 3 only.

### Compound 29

### 4-(1-(3,4-Difluoro-5-hydroxyphenyl)-1H-indazol-5-yl)-N-methyl-1-(methylsulfonyl)piperidine-4-carboxamide

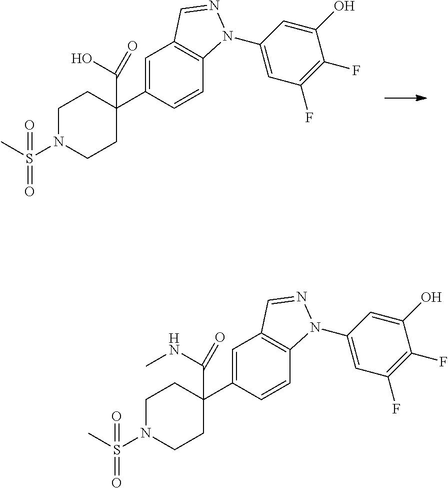 [SMILES: CS(=O)(=O)N1CCC(C(=O)O)(c2ccc3c(cnn3-c3cc(O)c(F)c(F)c3)c2)CC1 ; CNC(=O)C1(c2ccc3c(cnn3-c3cc(O)c(F)c(F)c3)c2)CCN(S(C)(=O)=O)CC1]

A mixture of Compound 28 (150 mg, 0.33 mmol), methanamine (67 mg, 0.10 mmol, HCl), HATU (164 mg, 0.432 mmol), DIEA (350 μL, 1.99 mmol), and DMF (10 mL) was stirred at room temperature for 2 h, poured into H₂O (50 mL), and then extracted (3×50 mL EtOAc). The combined organic layers were washed (50 mL brine), dried (Na₂SO₄), filtered, and then concentrated. The residue was purified by prep-HPLC [water (0.04% HCl)/CH₃CN] to give 4-(1-(3,4-difluoro-5-hydroxyphenyl)-1H-indazol-5-yl)-N-methyl-1l-(methylsulfonyl) piperidine-4-carboxamide (50 mg, 32%) as a white solid. ¹H NMR (400 MHz, DMSO-d₆): δ 10.89 (s, 1H), 8.37 (s, 1H), 7.86-7.78 (m, 2H), 7.69-7.61 (in, 1H), 7.52 (dd, 1H), 7.30-7.23 (m, 1H), 7.23-7.16 (m, 1H), 3.51-3.41 (m, 2H), 2.93 (t, 2H), 2.85 (s, 3H), 2.64 (d, 2H), 2.54 (d, 3H), 2.03-1.91 (m, 2H); LCMS: 465.0 [M+H]⁺.

The Compound below was synthesized from Compound 28 in a similar manner to that described for Compound 29.

| Cmpd | Structure | Name | [M + H]⁺ |
|---|---|---|---|
| 29.01 | 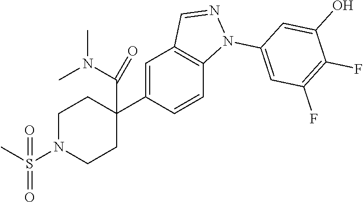 [SMILES: CN(C)C(=O)C1(c2ccc3c(cnn3-c3cc(O)c(F)c(F)c3)c2)CCN(S(C)(=O)=O)CC1] | 4-(1-(3,4-Difluoro-5-hydroxy- phenyl)-1H-indazol-5-yl)- N,N-dimethyl-1-(methyl- sulfonyl)piperidine-4-carbox- amide | 479.0 |

### Compound 30

### 2,3-Difluoro-5-(5-(piperidin-1-ylsulfonyl)-1H-indazol-1-yl)phenol

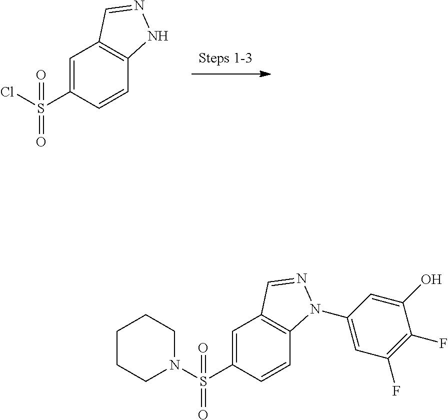 [SMILES: O=S(=O)(Cl)c1ccc2[nH]ncc2c1 ; O=S(=O)(c1ccc2c(cnn2-c2cc(O)c(F)c(F)c2)c1)N1CCCCC1]

### Step 1: 5-(Piperidin-1-ylsulfonyl)-1H-indazole

Piperidine (322 mg, 3.79 mmol) was added to a suspension of 1H-indazole-5-sulfonyl chloride (1.00 g, 1.89 mmol, 40% purity) and Et₃N (1.6 mL, 11.4 mmol) in DCM (15 mL). The resulting mixture was stirred at 20° C. for 1.5 h, concentrated, and then purified by prep-TLC (petroleum ether/EtOAc=4/1) to give 5-(piperidin-1-ylsulfonyl)-1H-indazole (365 mg, 73%) as a white solid. ¹H NMR (400 MHz, DMSO-d₆): δ 13.67 (br s, 1H), 8.32 (s, 1H), 8.25 (s, 1H), 7.75 (d, 1H), 7.66-7.61 (m, 1H), 2.87 (t, 4H), 1.54 (br s, 4H), 1.38-1.30 (m, 2H); LCMS: 266.1 [M+H]⁺.

### Step 2: 1-(3,4-Difluoro-5-(methoxymethoxy)phenyl)-5-(piperidin-1-ylsulfonyl)-1H-indazole

A mixture of 5-(piperidin-1-ylsulfonyl)-1H-indazole (215 mg, 0.81 mmol), Intermediate 2 (471 mg, 1.26 mmol, 80% purity), Cu(OAc)₂ (228 mg, 1.26 mmol), diethylamine (593 mg, 8.10 mmol), and THF (4 mL) was degassed and purged with oxygen 3 times, stirred for 14 h under an oxygen atmosphere, poured into concentrated NH₄OH (5 ml), and then extracted (3×10 mL EtOAc). The combined organic layers were washed (5 mL brine), dried (Na₂SO₄), filtered, and then concentrated. The residue was purified by prep-TLC (petroleum ether/EtOAc=3/1) to give 1-(3,4-difluoro-5-(methoxymethoxy)phenyl)-5-(piperidin-1-ylsulfonyl)-1H-indazole (65 mg, 18%) as a light yellow solid. ¹H NMR (400 MHz, DMSO-d₆): δ 8.63 (s, 1H), 8.38 (s, 1H), 8.05 (d, 1H), 7.79 (dd, 1H), 7.63-7.57 (m, 1H), 7.52 (d, 1H), 5.42 (s, 2H), 3.47-3.45 (m, 3H), 2.91 (t, 4H), 1.55 (br s, 4H), 1.33 (br s, 2H); LCMS: 438.1 [M+H]⁺.

### Step 3: 2,3-Difluoro-5-(5-(piperidin-1-ylsulfonyl)-1H-indazol-1-yl)phenol

Trifluoroacetic acid (0.5 ml, 6.8 mmol) was added to a mixture of 1-(3,4-difluoro-5-(methoxymethoxy)phenyl)-5-(piperidin-1-ylsulfonyl)-1H-indazole (65 mg, 0.15 mmol) and DCM (1 mL). The mixture was stirred at room temperature for 0.5 h, quenched by addition of saturated NaHCO₃ (5 mL), and then extracted (3×5 mL EtOAc). The combined organic layers were concentrated and purified by silica gel chromatography (20-50% EtOAc/petroleum ether) to give 2,3-difluoro-5-(5-(piperidin-1-ylsulfonyl)-1H-indazol-1-yl)phenol (14 mg, 24%) as a white solid. ¹H NMR (400 MHz, DMSO-d₆): δ 11.04 (br s, 1H), 8.60 (s, 1H), 8.37 (d, 1H), 8.01 (d, 1H), 7.79 (dd, 1H), 7.35-7.32 (m, 1H), 7.25-7.16 (m, 1H), 2.98-2.87 (m, 4H), 1.54 (d, 4H), 1.34 (d, 2H); LCMS: 394.0 [M+H]⁺.

The Compound below was synthesized from 1H-indazole-5-sulfonyl chloride and morpholine in a similar manner to that described for Compound 30.

| Cmpd | Structure | Name | [M + H]⁺ |
|---|---|---|---|
| 30.01 | 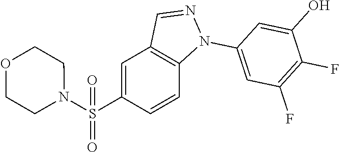 [SMILES: O=S(=O)(c1ccc2c(cnn2-c2cc(O)c(F)c(F)c2)c1)N1CCOCC1] | 2,3-Difluoro-5-(5-(morpho- linosulfonyl)-1H-indazol-1- yl)phenol | 395.9 |

### Compound 31

### 6-(5-(4-(Methylsulfonyl)piperazin-1-yl)-1H-indazol-1-yl)-4-(trifluoromethyl)pyridin-2-ol

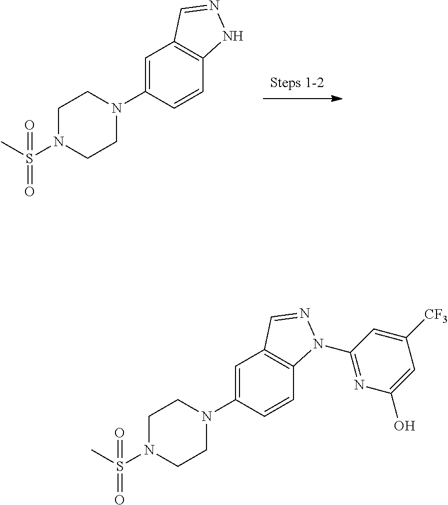 [SMILES: CS(=O)(=O)N1CCN(c2ccc3[nH]ncc3c2)CC1 ; CS(=O)(=O)N1CCN(c2ccc3c(cnn3-c3cc(C(F)(F)F)cc(O)n3)c2)CC1]

### Step 1: 1-(6-Chloro-4-(trifluoromethyl)pyridin-2-yl)-5-(4-(methylsulfonyl)piperazin-1-yl)-1H-indazole

A mixture of Intermediate 13.61 (700 mg, 2.50 mmol), 2,6-dichloro-4-(trifluoromethyl)pyridine (809 mg, 3.75 mmol), and Cs₂CO₃ (3.25 g, 9.99 mmol) in DMA (5 mL) was heated at 100° C. for 10 h, allowed to cool to room temperature, diluted with H₂O (20 mL), and then extracted (2×20 mL EtOAc). The combined organic layers were washed (20 mL brine), dried (Na₂SO₄), filtered, and then concentrated. The residue was purified by silica gel chromatography (2-100% EtOAc/petroleum ether) to give a 1.5:1 mixture of 1-(6-chloro-4-(trifluoromethyl)pyridin-2-yl)-5-(4-(methylsulfonyl)piperazin-1-yl)-1H-indazole and 2-(6-chloro-4-(trifluoromethyl)pyridin-2-yl)-5-(4-(methylsulfonyl)piperazin-1-yl)-2H-indazole (800 mg) as a green solid. ¹H NMR (400 MHz, DMSO-d₆): δ 8.42-8.51 (m, 2H), 8.12 (s, 1H), 7.83 (s, 1H), 7.49-7.55 (m, 1H), 7.32-7.34 (m, 1H), 3.30 (s, 8H), 2.95 (s, 3H); LCMS: 460.1 [M+H]⁺.

### Step 2: 6-(5-(4-(Methylsulfonyl)piperazin-1-yl)-1H-indazol-1-yl)-4-(trifluoromethyl)pyridin-2-ol

A solution of the 1.5:1 mixture of 1-(6-chloro-4-(trifluoromethyl)pyridin-2-yl)-5-(4-(methylsulfonyl)piperazin-1-yl)-1H-indazole and 2-(6-chloro-4-(trifluoromethyl)pyridin-2-yl)-5-(4-(methylsulfonyl)piperazin-1-yl)-2H-indazole (400 mg), KOH (122 mg, 2.17 mmol), and t-BuOH (4 mL) was heated at 90° C. overnight, allowed to cool to room temperature, poured into H₂O (20 mL), and then extracted (2×20 mL EtOAc). The combined organic layers were washed (20 mL brine), dried (Na₂SO₄), filtered, and then concentrated. The residue was purified by prep-HPLC [water (0.04% HCl)-MeCN] to give 6-(5-(4-(methylsulfonyl)piperazin-1-yl)-1H-indazol-1-yl)-4-(trifluoromethyl)pyridin-2-ol (25 mg, 13%) as a white solid. ¹H NMR (400 MHz, DMSO-d₆): δ 12.27 (s, 1H), 8.87 (d, 1H), 7.79 (s, 1H), 7.63 (d, 1H), 7.33 (d, 1H), 7.01 (d, 2H), 3.29-3.23 (m, 8H), 2.95 (s, 3H); LCMS: 442.0 [M+H]⁺.

The Compounds below were synthesized from Intermediate 13.61 in a similar manner to that described for Compound 31.

| Cmpd | Structure | Name | [M + H]⁺ |
|---|---|---|---|
| 31.01 (1) | 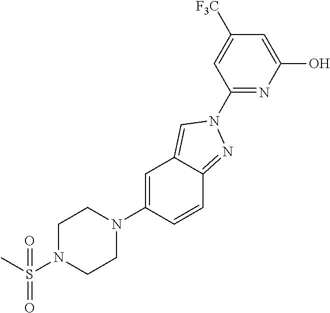 [SMILES: CS(=O)(=O)N1CCN(c2ccc3nn(-c4cc(C(F)(F)F)cc(O)n4)cc3c2)CC1] | 6-(5-(4-(Methylsulfonyl)- piperazin-1-yl)-2H-indazol- 2-yl)-4-(trifluoromethyl)- pyridin-2-ol | 442.0 |
| 31.02 (2) | 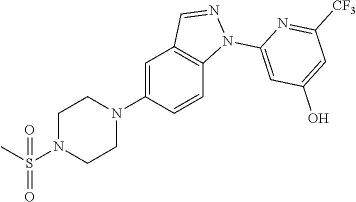 [SMILES: CS(=O)(=O)N1CCN(c2ccc3c(cnn3-c3cc(O)cc(C(F)(F)F)n3)c2)CC1] | 2-(5-(4-(Methylsulfonyl)- piperazin-1-yl)-1H-indazol- 1-yl)-6-(trifluoromethyl)- pyridin-4-ol | 442.0 |
| 31.03 (3) | 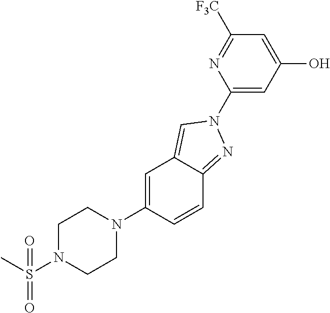 [SMILES: CS(=O)(=O)N1CCN(c2ccc3nn(-c4cc(O)cc(C(F)(F)F)n4)cc3c2)CC1] | 2-(5-(4-(Methylsulfonyl)- piperazin-1-yl)-2H-indazol- 2-yl)-6-(trifluoromethyl)- pyridin-4-ol | 442.0 |

Alternate conditions used: 1. Isolated from the synthesis of Compound 31; 2. Step 1: Synthesized using 2-chloro-4-iodo-6-(trifluoromethyl)pyridine and then Step 2 (NaOH, TBAF, H₂O, dioxane, 90° C., overnight); 3. Isolated from the synthesis of Compound 31.02.

### Example A-1: Parenteral Pharmaceutical Composition

To prepare a parenteral pharmaceutical composition suitable for administration by injection (subcutaneous, intravenous), 1-1000 mg of a compound described herein, or a pharmaceutically acceptable salt or solvate thereof, is dissolved in sterile water and then mixed with 10 mL of 0.9% sterile saline. A suitable buffer is optionally added as well as optional acid or base to adjust the pH. The mixture is incorporated into a dosage unit form suitable for administration by injection.

### Example A-2: Oral Solution

To prepare a pharmaceutical composition for oral delivery, a sufficient amount of a compound described herein, or a pharmaceutically acceptable salt thereof, is added to water (with optional solubilizer(s), optional buffer(s), and taste masking excipients) to provide a 20 mg/mL solution.

### Example A-3: Oral Tablet

A tablet is prepared by mixing 20-50% by weight of a compound described herein, or a pharmaceutically acceptable salt thereof, 20-50% by weight of microcrystalline cellulose, 1-10% by weight of low-substituted hydroxypropyl cellulose, and 1-10% by weight of magnesium stearate or other appropriate excipients. Tablets are prepared by direct compression. The total weight of the compressed tablets is maintained at 100-500 mg.

### Example A-4: Oral Capsule

To prepare a pharmaceutical composition for oral delivery, 10-500 mg of a compound described herein, or a pharmaceutically acceptable salt thereof, is mixed with starch or other suitable powder blend. The mixture is incorporated into an oral dosage unit such as a hard gelatin capsule, which is suitable for oral administration.

In another embodiment, 10-500 mg of a compound described herein, or a pharmaceutically acceptable salt thereof, is placed into size 4 capsule, or size 1 capsule (hypromellose or hard gelatin) and the capsule is closed.

### Example A-5: Topical Gel Composition

To prepare a pharmaceutical topical gel composition, a compound described herein, or a pharmaceutically acceptable salt thereof, is mixed with hydroxypropyl cellulose, propylene glycol, isopropyl myristate and purified alcohol USP. The resulting gel mixture is then incorporated into containers, such as tubes, which are suitable for topical administration.

### Example B-1: HSD17b13 NAD(P)H-Glo Biochemical Assay

### Materials

Recombinant human HSD17B13 enzyme. Substrate: estradiol (Sigma β-Estradiol E8875), 100 mM in DMSO. Cofactor: NAD+ Grade I free acid (Sigma 10127965001), 20 mM in H₂O. Assay buffer final concentration: 20 mM Tris pH7.4 with 0.002% Tween-20 and 0.02% BSA. Assay performed in 384 well solid bottom plate (Corning 3570). Enzymatic activity detected by NAD(P)H-Glo™ Detection System (Promega G9062).

### Compounds

Inhibitor compounds were serially diluted in DMSO and then further diluted in assay buffer to a 10× concentration consisting of 1% DMSO.

### Procedure

HSD17b13 enzyme was diluted in 1× assay buffer to the desired enzyme concentration based on the specific activity of the enzyme lot. 20 uL of diluted enzyme was added to each well along with 2.5 uL of 10× inhibitor solution. Assay plate was incubated at RT for 20 minutes, and then 2.5 uL of a 10× substrate/cofactor mix was added to each well for a final concentration of 50 uM estradiol and 1 mMNAD+. Assay plate was incubated at 37° C. for 3 hours. NAD(P)H-Glo™ Detection System reagents were prepared according to manufacturer's specifications, and 25 uL was added to each well. After incubating for 1 hour at RT, luminescence was measured.

Representative data for exemplary compounds disclosed herein is presented in Table 2.

TABLE 2

| | HSD17β13 |
|---|---|
| Cmpd | IC₅₀ (uM) |
| 1 | + |
| 1.01 | ++ |
| 1.02 | ++ |
| 1.03 | ++ |
| 1.04 | +++ |
| 1.05 | ++ |
| 1.06 | ++ |
| 2 | ++ |
| 2.01 | + |
| 3 | + |
| 3.01 | ++ |
| 3.02 | + |
| 3.03 | ++ |
| 3.04 | ++ |
| 3.05 | + |
| 4 | ++ |
| 4.01 | ++ |
| 4.02 | ++ |
| 4.03 | ++ |
| 4.04 | + |
| 4.05 | ++ |
| 4.06 | ++ |
| 4.07 | ++ |
| 4.08 | + |
| 4.09 | ++ |
| 4.10 | ++ |
| 4.11 | ++ |
| 4.12 | ++ |
| 4.13 | ++ |
| 4.14 | ++ |
| 4.15 | ++ |
| 4.16 | ++ |
| 4.17 | ++ |
| 5 | ++ |
| 5.01 | ++ |
| 5.02 | ++ |
| 5.03 | ++ |
| 5.04 | ++ |
| 5.05 | ++ |
| 5.06 | ++ |
| 5.07 | ++ |
| 5.08 | ++ |
| 5.09 | + |
| 5.10 | ++ |
| 5.11 | ++ |
| 5.12 | ++ |
| 5.13 | ++ |
| 5.14 | + |
| 5.15 | ++ |
| 5.16 | ++ |
| 5.17 | ++ |
| 5.18 | ++ |
| 5.19 | ++ |
| 5.20 | ++ |
| 5.21 | + |
| 5.22 | ++ |
| 5.23 | + |
| 5.24 | + |
| 5.25 | ++ |
| 5.26 | ++ |
| 5.27 | ++ |
| 5.28 | + |
| 5.29 | + |
| 5.30 | ++ |
| 6 | ++ |
| 6.01 | ++ |
| 7 | ++ |
| 7.01 | ++ |
| 7.02 | ++ |
| 7.03 | ++ |
| 7.04 | ++ |
| 8 | ++ |
| 8.01 | ++ |
| 8.02 | ++ |
| 8.03 | + |
| 9 | ++ |
| 10 | ++ |
| 11 | ++ |
| 11.01 | ++ |
| 11.02 | + |
| 12 | + |
| 13 | ++ |
| 14 | ++ |
| 15 | + |
| 16 | + |
| 16.01 | ++ |
| 16.02 | + |
| 16.03 | ++ |
| 17 | ++ |
| 18 | ++ |
| 18.01 | ++ |
| 18.02 | ++ |
| 19 | +++ |
| 19.01 | ++ |
| 19.02 | ++ |
| 19.03 | ++ |
| 19.04 | ++ |
| 19.05 | ++ |
| 19.06 | ++ |
| 19.07 | ++ |
| 19.08 | + |
| 19.09 | + |
| 19.10 | + |
| 19.11 | + |
| 19.12 | + |
| 19.13 | + |
| 19.14 | + |
| 19.15 | + |
| 19.16 | + |
| 19.17 | + |
| 19.18 | ++ |
| 19.19 | + |
| 19.20 | + |
| 19.21 | ++ |
| 19.22 | ++ |
| 19.23 | + |
| 19.24 | + |
| 19.25 | + |
| 19.26 | + |
| 19.27 | + |
| 19.28 | + |
| 19.29 | ++ |
| 19.30 | ++ |
| 19.31 | + |
| 19.32 | ++ |
| 19.33 | ++ |
| 19.34 | ++ |
| 19.35 | 49% |
| | inhibition |
| | at 30 uM |
| 19.36 | 40% |
| | inhibition |
| | at 30 uM |
| 19.37 | 43% |
| | inhibition |
| | at 30 uM |
| 19.38 | ++ |
| 19.39 | + |
| 19.40 | 39% |
| | inhibition |
| | at 30 uM |
| 19.41 | + |
| 19.42 | + |
| 19.43 | 45% |
| | inhibition |
| | at 30 uM |
| 19.44 | +++ |
| 19.45 | ++ |
| 19.46 | +++ |
| 19.47 | +++ |
| 19.48 | ++ |
| 19.49 | +++ |
| 19.50 | +++ |
| 19.51 | ++ |
| 19.52 | +++ |
| 19.53 | +++ |
| 19.54 | +++ |
| 19.55 | ++ |
| 19.56 | +++ |
| 19.57 | +++ |
| 19.58 | +++ |
| 19.59 | ++ |
| 19.60 | ++ |
| 19.61 | 55% |
| | inhibition |
| | at 30 uM |
| 19.62 | ++ |
| 19.63 | ++ |
| 19.64 | ++ |
| 19.65 | +++ |
| 19.66 | +++ |
| 19.67 | +++ |
| 19.68 | +++ |
| 19.69 | +++ |
| 19.70 | +++ |
| 19.71 | ++ |
| 19.72 | ++ |
| 19.73 | ++ |
| 19.74 | ++ |
| 19.75 | ++ |
| 19.76 | ++ |
| 19.77 | +++ |
| 19.78 | +++ |
| 19.79 | +++ |
| 19.80 | ++ |
| 19.81 | +++ |
| 19.82 | +++ |
| 19.83 | +++ |
| 19.84 | +++ |
| 19.85 | +++ |
| 19.86 | ++ |
| 19.87 | ++ |
| 19.88 | ++ |
| 19.89 | + |
| 19.90 | ++ |
| 19.91 | ++ |
| 19.92 | + |
| 19.93 | ++ |
| 19.94 | +++ |
| 19.95 | ++ |
| 19.96 | + |
| 19.97 | + |
| 19.98 | ++ |
| 19.99 | ++ |
| 19.100 | +++ |
| 19.101 | ++ |
| 19.102 | ++ |
| 19.103 | ++ |
| 19.104 | +++ |
| 19.105 | +++ |
| 19.106 | ++ |
| 19.107 | +++ |
| 19.108 | +++ |
| 19.109 | ++ |
| 19.110 | +++ |
| 19.111 | ++ |
| 19.112 | + |
| 19.113 | + |
| 19.114 | +++ |
| 19.115 | +++ |
| 19.116 | +++ |
| 19.117 | ++ |
| 19.118 | + |
| 19.119 | + |
| 19.120 | 25% |
| | inhibition |
| | at 10 uM |
| 19.121 | <20% |
| | inhibition |
| | at 10 uM |
| 19.122 | 20% |
| | inhibition |
| | at 10 uM |
| 19.123 | 30% |
| | inhibition |
| | at 30 uM |
| 20 | + |
| 20.01 | + |
| 20.02 | + |
| 20.03 | 40% |
| | inhibition |
| | at 100 uM |
| 20.04 | <20% |
| | inhibition |
| | at 10 uM |
| 20.05 | <20% |
| | inhibition |
| | at 100 uM |
| 21 | ++ |
| 21.01 | ++ |
| 21.02 | + |
| 21.03 | + |
| 21.04 | ++ |
| 21.05 | + |
| 21.06 | + |
| 21.07 | + |
| 21.08 | + |
| 21.09 | ++ |
| 21.10 | ++ |
| 21.11 | + |
| 21.12 | + |
| 21.13 | ++ |
| 21.14 | + |
| 21.15 | + |
| 21.16 | + |
| 21.17 | 47% |
| | inhibition |
| | at 30 uM |
| 21.18 | + |
| 21.19 | + |
| 21.20 | ++ |
| 21.21 | ++ |
| 21.22 | + |
| 21.23 | ++ |
| 21.24 | ++ |
| 21.25 | + |
| 21.26 | ++ |
| 21.27 | ++ |
| 21.28 | ++ |
| 21.29 | ++ |
| 21.30 | + |
| 21.31 | ++ |
| 21.32 | ++ |
| 21.33 | ++ |
| 21.34 | ++ |
| 21.35 | + |
| 21.36 | ++ |
| 21.37 | ++ |
| 21.38 | ++ |
| 21.39 | ++ |
| 21.40 | ++ |
| 21.41 | ++ |
| 21.42 | ++ |
| 21.43 | ++ |
| 21.44 | +++ |
| 21.45 | ++ |
| 21.46 | ++ |
| 21.47 | ++ |
| 21.48 | + |
| 21.49 | ++ |
| 21.50 | ++ |
| 21.51 | ++ |
| 21.52 | ++ |
| 21.53 | +++ |
| 21.54 | ++ |
| 21.55 | ++ |
| 21.56 | ++ |
| 21.57 | ++ |
| 21.58 | ++ |
| 21.59 | ++ |
| 21.60 | + |
| 21.61 | <20% |
| | inhibition |
| | at 30 uM |
| 21.62 | <20% |
| | inhibition |
| | at 30 uM |
| 21.63 | <20% |
| | inhibition |
| | at 10 uM |
| 22 | ++ |
| 23 | ++ |
| 24 | + |
| 24.01 | ++ |
| 25 | ++ |
| 25.01 | + |
| 26 | + |
| 26.01 | + |
| 26.02 | ++ |
| 27 | +++ |
| 27.01 | + |
| 28 | 49% |
| | inhibition |
| | at 30 uM |
| 28.01 | + |
| 28.02 | ++ |
| 28.03 | + |
| 28.04 | + |
| 28.05 | + |
| 28.06 | + |
| 28.07 | ++ |
| 28.08 | + |
| 29 | + |
| 29.01 | <20% |
| | inhibition |
| | at 10 uM |
| 30 | + |
| 30.01 | ++ |
| 31 | ++ |
| 31.01 | + |
| 31.02 | + |
| 31.03 | + |

where ‘+++’ means IC₅₀ ≤ 0.1 uM; where ‘++’ means 0.1 uM < IC₅₀ ≤ 1 uM; where ‘+’ means 1.0 uM < IC₅₀ ≤ 30 uM.

### Example B-2: HSD17b1 NAD(P)H-Glo Biochemical Assay

### Materials

Recombinant human HSD17B1 enzyme. Substrate: testosterone (Sigma T1500), 100 mM in DMSO. Cofactor: NADP disodium salt (Sigma 10128031001), 20 mM in H₂O. Assay buffer final concentration: 20 mM Tris pH7.4 with 0.002% Tween-20 and 0.02% BSA. Assay performed in 384 well solid bottom plate (Corning 3570). Enzymatic activity detected by NAD(P)H-Glo™ Detection System (Promega G9062).

### Compounds

Inhibitor compounds were serially diluted in DMSO and then further diluted in assay buffer to a 10× concentration consisting of 10% DMSO.

### Procedure

HSD17b 1 enzyme was diluted in IX assay buffer to the desired enzyme concentration based on the specific activity of the enzyme lot. 20 uL of diluted enzyme was added to each well along with 2.5 uL of the 10× inhibitor solution. Assay plate was incubated at RT for 20 minutes, and then 2.5 uL of a 10× substrate/cofactor mix was added to each well for a final concentration of 55 uM testosterone and 1 mM NADP. Assay plate was incubated at 37° C. for 1 hour. NAD(P)H-Glo™ Detection System reagents were prepared according to manufacturer's specifications, and 25 uL was added to each well. After incubating for 1 hour at RT, luminescence was measured.

### Example B-3: HSD17b2 NAD(P)H-Glo Biochemical Assay

### Materials and Setup

Recombinant human HSD17B2 enzyme. Substrate: estradiol (Sigma β-Estradiol E8875) 2 mM in DMSO. Cofactor: NAD+Grade I free acid (Sigma 10127965001), 20 mM in H₂O. Assay buffer final concentration: 20 mM Tris pH7.4 with 0.002% Tween-20 and 0.02% BSA. Assay performed in 384 well solid bottom plate (Corning 3570). Enzymatic activity detected by NAD(P)H-Glo™ Detection System (Promega G9062).

### Compounds

Inhibitor compounds were serially diluted in DMSO and then further diluted in assay buffer to a 10× concentration consisting of 1% DMSO.

### Procedure

HSD17b2 enzyme was diluted in 1× assay buffer to the desired enzyme concentration based on the specific activity of the enzyme lot. 20 uL of diluted enzyme was added to each well along with 2.5 uL of 10× inhibitor solution. Assay plate was incubated at RT for 20 minutes, and then 2.5 uL of 10× substrate/cofactor mix was added to each well for a final assay concentration of 1 uM estradiol and 500 uM NAD+. Assay plate was incubated at RT for 1 hour. NAD(P)H-Glo™ Detection System reagents were prepared according to manufacturer's specifications and 25 uL was added to each well. After incubating for 1 hour at RT, luminescence was measured.

### Example B-4: In Vitro HSD17b13 Cell Based Assay

### Seeding

HEK293 cells were plated at 4,000,000 cells per T75 flask with EMEM (ATCC Cat #30-2003) and 10% FBS (Sigma Cat #F2442) and then incubated at 37° C. in 5% CO₂ for 18 hours.

### Transfection and Plate

After the 18 h incubation, media was replaced with 15 mL of fresh media: EMEM without Phenol Red (Quality Biological Cat #112-212-101), 10% CSS (Sigma Cat #F6765) and GlutaMax (Gibco Cat #35050-061). In a polypropylene tube, 20 ug pCMV6 HSD17B13 (Origene Cat #RC213132) was diluted in OptiMEM (Life Technologies, Cat #31985-062) to 2 mL. 60 uL of transfection reagent (X-tremeGENE HP Roche, Cat #06 366 236 001) was added, and the tube was vortexed and incubated at room temperature for 20 minutes. The transfection reagent/DNA mixture was added to the cells in the T75 flask, and the cells were incubated at 37° C. in 5% CO₂ for 18 hours. The next day, the cells were resuspended in EMEM media with 10% CSS and plated in a 96 well plate at 80,000 cells/well, 100 uL/well. Cells were incubated at 37° C. in 5% CO₂ for 18 hours.

### Test Compounds

Compounds were serially diluted in DMSO (1000× final concentration) and then further diluted in EMEM media with 10% CSS to a 20× final concentration. 10 uL of the 20× compound mix was added to each well of transfected cells, and the cells were incubated at 37° C. in 5% CO₂ for 30 minutes. 100 uL of EMEM media with 100 uM estradiol (Sigma cat #E8875) was added to each well, and the cells were incubated for 4 hours at 37° C. in 5% CO₂. The cell media was collected and examined for estradiol and estrone concentrations by LCMS.

### Example B-5: In Vitro HSD17b11 Cell Based Assay

### Seeding

HEK293 cells were plated at 4,000,000 cells per T75 flask with EMEM (ATCC Cat #30-2003) and 10% FBS (Sigma Cat #F2442) and then incubated at 37° C. in 5% CO₂ for 18 hours.

### Transfection and Plate

After the 18 h incubation, the media was replaced with 15 mL of fresh media: EMEM without Phenol Red (Quality Biological Cat #112-212-101), 10% CSS (Sigma Cat #F6765) and GlutaMax (Gibco Cat #35050-061). In a polypropylene tube, 20 ug pCMV6 HSD17B11 (Origene Cat #RC205941) was diluted in OptiMEM (Life Technologies, Cat #31985-062) to 2 mL. 60 uL of transfection reagent (X-tremeGENE HP Roche, Cat #06 366 236 001) was added, and the tube was vortexed and incubated at room temperature for 20 minutes. The transfection reagent/DNA mixture was added to the cells in the T75 flask, and the cells were incubated at 37° C. in 5% CO₂ for 18 hours. The next day, the transfected cells were resuspended in EMEM media with 10% CSS and plated in a 96 well plate at 80,000 cells/well, 100 uL/well. Cells were incubated at 37° C. in 5% CO₂ for 18 hours.

### Test Compounds

Compounds were serially diluted in DMSO (1000× final concentration) and then further diluted in EMEM media with 10% CSS to a 20× final concentration. 10 uL of the 20× compound mix was added to each well of the transfected cells, and the cells were incubated at 37° C. in 5% CO₂ for 30 minutes. 100 uL of EMEM media with 60 uM of estradiol (Sigma cat #E8875) was added, and the cells were incubated for 4 hours at 37° C. in 5% CO₂. The cell media was examined for estradiol and estrone concentrations by LCMS.

### Example B-6: NASH Activity Study (AMLN Model)

NASH is induced in male C₅₇BL/6 mice by diet-induction with AMLN diet (DIO-NASH) (D09100301, Research Diet, USA) (40% fat (18% trans-fat), 40% carbohydrates (20% fructose) and 2% cholesterol). The animals are kept on the diet for 29 weeks. After 26 weeks of diet induction, liver biopsies are performed for base line histological assessment of disease progression (hepatosteatosis and fibrosis), stratified and randomized into treatment groups according to liver fibrosis stage, steatosis score, and body weight. Three weeks after biopsy the mice are stratified into treatment groups and dosed daily by oral gavage with an HSD17B13 inhibitor for 8 weeks. At the end of the study liver biopsies are performed to assess hepatic steatosis and fibrosis by examining tissue sections stained with H&E and Sirius Red, respectively. Total collagen content in the liver is measured by colorimetric determination of hydroxyproline residues by acid hydrolysis of collagen. Triglycerides and total cholesterol content in liver homogenates are measured in single determinations using auto analyzer Cobas C-111 with commercial kit (Roche Diagnostics, Germany) according to manufacturer's instructions.

### Example B-7: CCl₄ Fibrosis Model

Fibrosis is induced in C57BL/6 male mice by bi-weekly oral administration of CCl₄. CCl₄ is formulated 1:4 in oil and is oral dosed at a final concentration of 0.5 ul/g mouse. After 2-4 weeks of fibrosis induction the compounds is administered daily by oral gavage for 2-8 weeks of treatment while continuing CCl₄ administration. At study termination livers are formalin fixed and stained with H&E or Sirius Red stain for histopathological evaluation of inflammation and fibrosis. Total collagen content is measured by colorimetric determination of hydroxyproline residues by acid hydrolysis of collagen. Collagen gene induction is measured by qPCR analysis of Col1a1 and Col3a1 mRNA. Serum alanine aminotransferase (ALT) and aspartate aminotransferase (AST) are measured by a clinical chemistry analyzer.

### Example B-8: Mouse PK Study

The plasma pharmacokinetics of any one of the compounds disclosed herein as a test article is measured following a single bolus intravenous and oral administration to mice (CD-1, C57BL, and diet induced obesity mice). Test article is formulated for intravenous administration in a vehicle solution of DMSO, PEG400, hydroxypropyl-β-cyclodextrin (HPPCD) and is administered (for example at a dose volume of 3 mL/kg) at selected dose levels. An oral dosing formulation is prepared in appropriate oral dosing vehicles (vegetable oils, PEG400, Solutol, citrate buffer, or carboxymethyl cellulose) and is administered at a dose volume of 5-10 mL/kg at selected dose levels. Blood samples (approximately 0.15 mL) are collected by cheek pouch method at pre-determined time intervals post intravenous or oral doses into tubes containing EDTA. Plasma is isolated by centrifugation of blood at 10,000 g for 5 minutes, and aliquots are transferred into a 96-well plate and stored at −60° C. or below until analysis.

Calibration standards of test article are prepared by diluting DMSO stock solution with DMSO in a concentration range. Aliquots of calibration standards in DMSO are combined with plasma from naïve mouse so that the final concentrations of calibration standards in plasma are 10-fold lower than the calibration standards in DMSO. PK plasma samples are combined with blank DMSO to match the matrix. The calibration standards and PK samples are combined with ice-cold acetonitrile containing an analytical internal standard and centrifuged at 1850 g for 30 minutes at 4° C. The supernatant fractions are analyzed by LC/MS/MS and quantitated against the calibration curve. Pharmacokinetic parameters (area under the curve (AUC), Cₘₐₓ, Tₘₐₓ, elimination half-life (T_{U}m), clearance (CL), steady state volume of distribution (V_{dss}), and mean residence time (MRT)) are calculated via non-compartmental analysis using Microsoft Excel (version 2013).

### Example B-9: Mouse CDA-HFD NASH Model

A NASH phenotype with mild fibrosis can be induced in C₅₇BL/6 mice by feeding a choline-deficient diet with 0.1% methionine and 60% kcal fat (Research Diet A06071302) for 4-12 weeks. After 4-6 weeks of diet induction compounds can be administered daily by oral gavage for 4-8 weeks of treatment while continuing CDA-HFD feeding. At study termination livers can be formalin fixed and stained with H&E and Sirius Red stain histopathological evaluation of steatosis, inflammation, and fibrosis. Total collagen content can be measured by colorimetric determination of hydroxyproline residues by acid hydrolysis of collagen. Collagen gene induction can be measured by qPCR analysis of Col1a1 or Col3a1. Serum alanine aminotransferase (ALT) and aspartate aminotransferase (AST) can be measured by a clinical chemistry analyzer.

What is claimed is:

1. A compound of Formula (I′), or a pharmaceutically acceptable salt or solvate thereof: 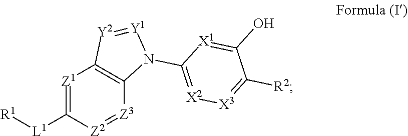
wherein:
X¹, X², and X³ are each independently CR³ or N;
Y¹ and Y² are each independently CR⁴ or N;
Z¹, Z², and Z³ are each independently CR⁵ or N;
L¹ is selected from a bond, —O—, —N(R¹⁰)—, —S(O)₂—, —C(R¹⁰)(R¹¹)N(R¹⁰)—, and —N(R¹⁰)C(R¹⁰)(R¹¹)—;
R¹ is selected from:
a) C₃₋₈cycloalkyl and C₂₋₉heterocycloalkyl, wherein C₃₋₈cycloalkyl and C₂₋₉heterocycloalkyl are optionally substituted with one, two, or three R⁶; or
b) C₆₋₁₀aryl and C₁₋₉heteroaryl, wherein C₆₋₁₀aryl and C₁₋₉heteroaryl are substituted with one, two, or three R⁷;
R² is selected from H, halogen, —CN, C₁₋₆alkyl, C₁₋₆haloalkyl, C₂₋₆alkenyl, C₂₋₆alkynyl, C₃₋₆ cycloalkyl, C₂₋₉heterocycloalkyl, C₆₋₁₀aryl, C₁₋₉heteroaryl, —OR¹⁰, —SR¹⁰, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)C(O)N(R¹⁰)(R¹¹), —N(R¹²)C(O)R¹³, —S(O)₂R¹³, —S(O)₂N(R¹⁰)(R¹¹)—, S(═O)(═NH)N(R¹⁰)(R¹¹), —CH₂C(O)N(R¹⁰)(R¹¹), —CH₂N(R¹²)C(O)R¹³, —CH₂S(O)₂R¹³, and —CH₂S(O)₂N(R¹⁰)(R¹¹), wherein C₁₋₆alkyl, C₂₋₆alkenyl, C₂₋₆alkynyl, C₃₋₆ cycloalkyl, C₂₋₉heterocycloalkyl, C₆₋₁₀aryl, and C₁₋₉heteroaryl are optionally substituted with one, two, or three groups selected from halogen, C₁₋₆alkyl, C₁₋₆haloalkyl, —OR¹⁰, and —N(R¹⁰)(R¹¹);
each R³ and each R⁴ are each independently selected from H, halogen, —CN, C₁₋₆alkyl, C₁₋₆ haloalkyl, C₂₋₆alkenyl, C₂₋₆alkynyl, C₃₋₆cycloalkyl, C₂₋₉heterocycloalkyl, C₁₋₉ heteroaryl, —OR¹⁰, —SR¹⁰, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R¹³, —S(O)₂N(R¹⁰)(R¹¹)—, S(═O)(═NH)N(R¹⁰)(R¹¹), —CH₂C(O)N(R¹⁰)(R¹¹), —CH₂N(R¹²)C(O)R¹³, —CH₂S(O)₂R¹³, and —CH₂S(O)₂N(R¹⁰)(R¹¹), wherein C₁₋₆alkyl, C₂₋₆alkenyl, C₂₋₆alkynyl, C₃₋₆cycloalkyl, C₂₋₉heterocycloalkyl, and C₁₋₉heteroaryl are optionally substituted with one, two, or three groups selected from halogen, C₁₋₆alkyl, C₁₋₆haloalkyl, —OR¹⁰, and —N(R¹⁰)(R¹¹);
each R⁵ is independently selected from H, halogen, —CN, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹⁰, —SR¹⁰, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R¹³, —S(O)₂N(R¹⁰)(R¹¹)—, S(═O)(═NH)N(R¹⁰)(R¹¹), —CH₂C(O)N(R¹⁰)(R¹¹), —CH₂N(R¹²)C(O)R¹³, —CH₂S(O)₂R¹³, and —CH₂S(O)₂N(R¹⁰)(R¹¹), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from halogen, C₁₋₆alkyl, C₁₋₆haloalkyl, —OR¹⁰, and —N(R¹⁰)(R¹¹);
each R⁶ and each R⁷ are each independently selected from halogen, oxo, —CN, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹⁰, —SR¹⁰, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R¹³, —S(O)₂N(R¹⁰)(R¹¹)—, S(═O)(═NH)N(R¹⁰)(R¹¹), —CH₂C(O)N(R¹⁰)(R¹¹), —CH₂N(R¹²)C(O)R¹³, —CH₂S(O)₂R¹³, and —CH₂S(O)₂N(R¹⁰)(R¹¹), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three groups selected from halogen, C₁₋₆alkyl, C₁₋₆haloalkyl, —OR¹⁰, —C(O)OR¹⁰, and —N(R¹⁰)(R¹¹);
each R¹⁰ is independently selected from hydrogen, C₁₋₆alkyl, C₁₋₆ haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from halogen, C₁₋₆alkyl, C₁₋₆haloalkyl, C₁₋₆alkoxy, C₃₋₆cycloalkyl, C₂₋₉heterocycloalkyl, C₆₋₁₀aryl, C₁₋₉heteroaryl, —N(R¹¹)(R¹²), and —C(O)OR¹¹;
each R¹¹ is independently selected from hydrogen, C₁₋₆alkyl, and C₁₋₆haloalkyl;
each R¹² is independently selected from hydrogen, C₁₋₆alkyl, and C₁₋₆haloalkyl; and
each R¹³ is independently selected C₁₋₆alkyl, C₁₋₆haloalkyl, C₂₋₆alkenyl, C₂₋₆alkynyl, C₃₋₆ cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three groups selected from halogen, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl.

2. A compound of Formula (II), or a pharmaceutically acceptable salt or solvate thereof: 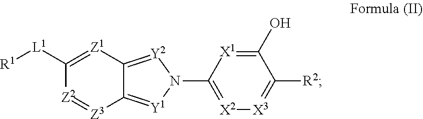
wherein:
X¹, X², and X³ are each independently CR³ or N;
Y¹ and Y² are each independently CR⁴ or N;
Z¹, Z², and Z³ are each independently CR⁵ or N;
L¹ is selected from a bond, —O—, and —N(R¹⁰)—;
R¹ is selected from:
a) C₃₋₈cycloalkyl and C₂₋₉heterocycloalkyl, wherein C₃₋₈cycloalkyl and C₂₋₉heterocycloalkyl are optionally substituted with one, two, or three R⁶; or
b) C₆₋₁₀aryl and C₁₋₉heteroaryl, wherein C₆₋₁₀aryl and C₁₋₉heteroaryl are substituted with one, two, or three R⁷;
R² is selected from H, halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹⁰, —SR¹⁰, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R¹³, —S(O)₂N(R¹⁰)(R¹¹)—, S(═O)(═NH)N(R¹⁰)(R¹¹), —CH₂C(O)N(R¹⁰)(R¹¹), —CH₂N(R¹²)C(O)R¹³, —CH₂S(O)₂R¹³, and —CH₂S(O)₂N(R¹⁰)(R¹¹), wherein C₁₋₆alkyl, C₂₋₆alkenyl, C₂₋₆alkynyl, C₃₋₆ cycloalkyl, C₂₋₉heterocycloalkyl, C₆₋₁₀aryl, and C₁₋₉heteroaryl are optionally substituted with one, two, or three groups selected from halogen, C₁₋₆alkyl, C₁₋₆ haloalkyl, —OR¹⁰, and —N(R¹⁰)(R¹¹);
each R³ is independently selected from H, halogen, —CN, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹⁰, —SR¹⁰, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R¹³, —S(O)₂N(R¹⁰)(R¹¹)—, S(═O)(═NH)N(R¹⁰)(R¹¹), —CH₂C(O)N(R¹⁰)(R¹¹), —CH₂N(R¹²)C(O)R¹³, —CH₂S(O)₂R¹³, and —CH₂S(O)₂N(R¹⁰)(R¹¹), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three groups selected from halogen, C₁₋₆alkyl, C₁₋₆haloalkyl, —OR¹⁰, and —N(R¹⁰)(R¹¹);
each R⁴ and each R⁵ are each independently selected from H, halogen, —CN, C₁₋₆alkyl, C₁₋₆ haloalkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —SR¹⁰, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R¹³, —S(O)₂N(R¹⁰)(R¹¹)—, S(═O)(═NH)N(R¹⁰)(R¹¹), —CH₂C(O)N(R¹⁰)(R¹¹), —CH₂N(R¹²)C(O)R¹³, —CH₂S(O)₂R¹³, and —CH₂S(O)₂N(R¹⁰)(R¹¹), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three groups selected from halogen, C₁₋₆alkyl, C₁₋₆haloalkyl, —OR¹⁰, and —N(R¹⁰)(R¹¹);
each R⁶ and each R⁷ are each independently selected from halogen, oxo, —CN, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR¹⁰, —SR¹⁰, —N(R¹⁰)(R¹¹), —C(O)OR¹⁰, —OC(O)N(R¹⁰)(R¹¹), —N(R¹²)C(O)N(R¹⁰)(R¹¹), —N(R¹²)C(O)OR¹³, —N(R¹²)S(O)₂R¹³, —C(O)R¹³, —S(O)R¹³, —OC(O)R¹³, —C(O)N(R¹⁰)(R¹¹), —C(O)C(O)N(R¹⁰)(R¹¹), —N(R¹²)C(O)R¹³, —S(O)₂R¹³, —S(O)₂N(R¹⁰)(R¹¹)—, S(═O)(═NH)N(R¹⁰)(R¹¹), —CH₂C(O)N(R¹⁰)(R¹¹), —CH₂N(R¹²)C(O)R¹³, —CH₂S(O)₂R¹³, and —CH₂S(O)₂N(R¹⁰)(R¹¹), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉ heteroaryl are optionally substituted with one, two, or three groups selected from halogen, C₁₋₆alkyl, C₁₋₆haloalkyl, —OR¹⁰, and —N(R¹⁰)(R¹¹);
each R¹⁰ is independently selected from hydrogen, C₁₋₆alkyl, C₁₋₆ haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from halogen, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl;
each R¹¹ is independently selected from hydrogen, C₁₋₆alkyl, and C₁₋₆haloalkyl;
each R¹² is independently selected from hydrogen, C₁₋₆alkyl, and C₁₋₆haloalkyl; and
each R¹³ is independently selected C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three groups selected from halogen, C₁₋₆alkyl, C₁₋₆haloalkyl, C₁₋₆alkoxy, C₃₋₆cycloalkyl, C₂₋₉heterocycloalkyl, C₆₋₁₀aryl, and C₁₋₉heteroaryl.

3. The compound of claim 1 or claim 2, or a pharmaceutically acceptable salt or solvate thereof, wherein X¹, X², and X³ are CR³.

4. The compound of any one of claims 1-3, or a pharmaceutically acceptable salt or solvate thereof, wherein Y² is CR⁴.

5. The compound of claim 1, or a pharmaceutically acceptable salt or solvate thereof, having the structure of Formula (Ia′):

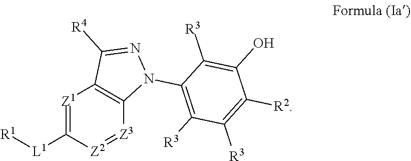

6. The compound of any one of claims 1-5, or a pharmaceutically acceptable salt or solvate thereof, wherein Z¹, Z², and Z³ are CR⁵.

7. The compound of any one of claims 1-5, or a pharmaceutically acceptable salt or solvate thereof, wherein Z¹ is N; and Z² and Z³ are CR⁵.

8. The compound of any one of claims 1-5, or a pharmaceutically acceptable salt or solvate thereof, wherein Z² is N; and Z¹ and Z³ are CR⁵.

9. The compound of any one of claims 1-5, or a pharmaceutically acceptable salt or solvate thereof, wherein Z³ is N; and Z¹ and Z² are CR⁵.

10. The compound of any one of claims 1-5, or a pharmaceutically acceptable salt or solvate thereof, wherein Z¹ is CR⁵; and Z² and Z³ are N.

11. The compound of any one of claims 1-5, or a pharmaceutically acceptable salt or solvate thereof, wherein Z² is CR⁵; and Z¹ and Z³ are N.

12. The compound of any one of claims 1-5, or a pharmaceutically acceptable salt or solvate thereof, wherein Z³ is CR⁵; and Z¹ and Z² are N.

13. The compound of any one of claims 1-12, or a pharmaceutically acceptable salt or solvate thereof, wherein L¹ is a bond.

14. The compound of any one of claims 1-12, or a pharmaceutically acceptable salt or solvate thereof, wherein L¹ is —O—.

15. The compound of any one of claims 1-12, or a pharmaceutically acceptable salt or solvate thereof, wherein L¹ is —N(R¹⁰)—.

16. The compound of any one of claims 1-12, or a pharmaceutically acceptable salt or solvate thereof, wherein L¹ is —N(H)—.

17. The compound of any one of claims 1-12, or a pharmaceutically acceptable salt or solvate thereof, wherein L¹ is —N(CH₃)—.

18. The compound of any one of claims 1-17, or a pharmaceutically acceptable salt or solvate thereof, wherein R¹ is selected from C₃₋₈cycloalkyl and C₂₋₉heterocycloalkyl, wherein C₃₋₈cycloalkyl and C₂₋₉heterocycloalkyl are optionally substituted with one, two, or three R⁶.

19. The compound of any one of claims 1-18, or a pharmaceutically acceptable salt or solvate thereof, wherein R¹ is C₂₋₉heterocycloalkyl optionally substituted with one, two, or three R⁶.

20. The compound of any one of claims 1-19, or a pharmaceutically acceptable salt or solvate thereof, wherein R¹ is C₂₋₉heterocycloalkyl selected from piperidinyl, piperazinyl, morpholinyl, tetrahydropyranyl, tetrahydrofuranyl, pyrrolidinyl, oxetanyl, azetidinyl, aziridinyl, azepanyl, diazepanyl, 6-azaspiro[2.5]octanyl, 4,7-diazaspiro[2.5]octanyl, 7-oxa-4-azaspiro[2.5]octanyl, 5,8-diazaspiro[3.5]nonanyl, 8-oxa-5-azaspiro[3.5]nonanyl, or 2,6-diazaspiro[3.3]heptanyl, wherein piperidinyl, piperazinyl, morpholinyl, tetrahydropyranyl, tetrahydrofuranyl, pyrrolidinyl, oxetanyl, azetidinyl, aziridinyl, azepanyl, diazepanyl, 6-azaspiro[2.5]octanyl, 4,7-diazaspiro[2.5]octanyl, 7-oxa-4-azaspiro[2.5]octanyl, 5,8-diazaspiro[3.5]nonanyl, 8-oxa-5-azaspiro[3.5]nonanyl, or 2,6-diazaspiro[3.3]heptanyl are optionally substituted with one, two, or three R⁶.

21. The compound of any one of claims 1-20, or a pharmaceutically acceptable salt or solvate thereof, wherein R¹ is

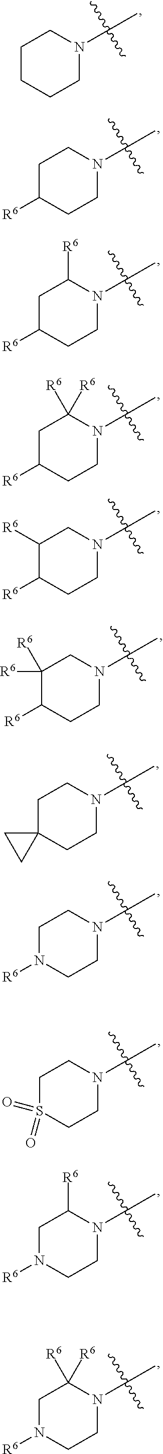

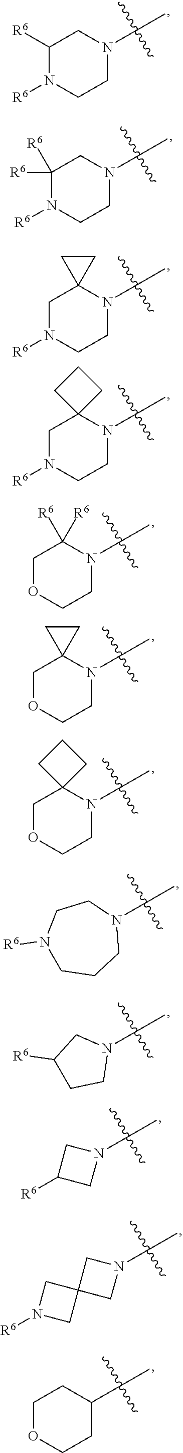

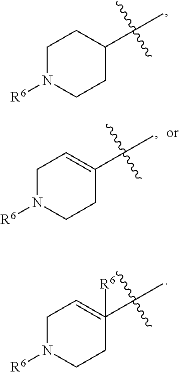

22. The compound of any one of claims 1-21, or a pharmaceutically acceptable salt or solvate thereof, wherein each R⁶ is independently selected from C₁₋₆alkyl, —OR¹⁰, —C(O)OR¹⁰, —N(R¹²)S(O)₂R¹³, —C(O)R¹³, —C(O)N(R¹⁰)(R¹¹), —S(O)₂R¹³, and —S(O)₂N(R¹⁰)(R¹¹)—.

23. The compound of any one of claims 1-22, or a pharmaceutically acceptable salt or solvate thereof, wherein R¹ is

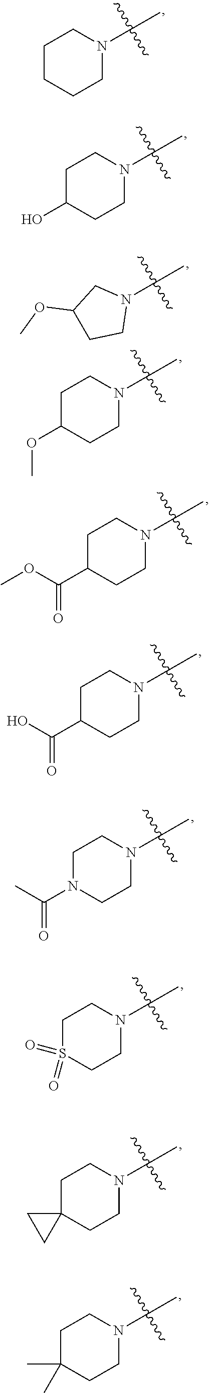

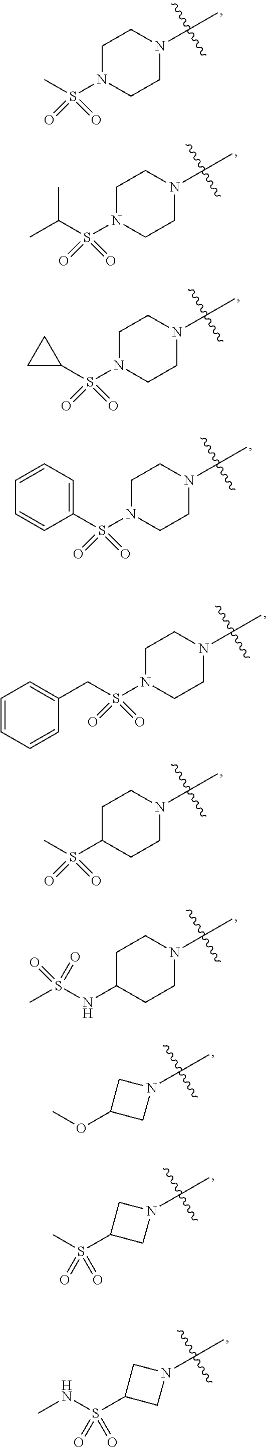

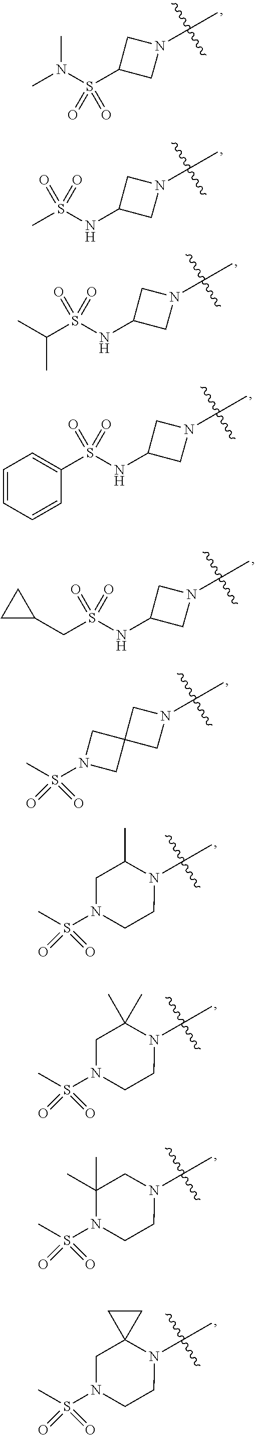

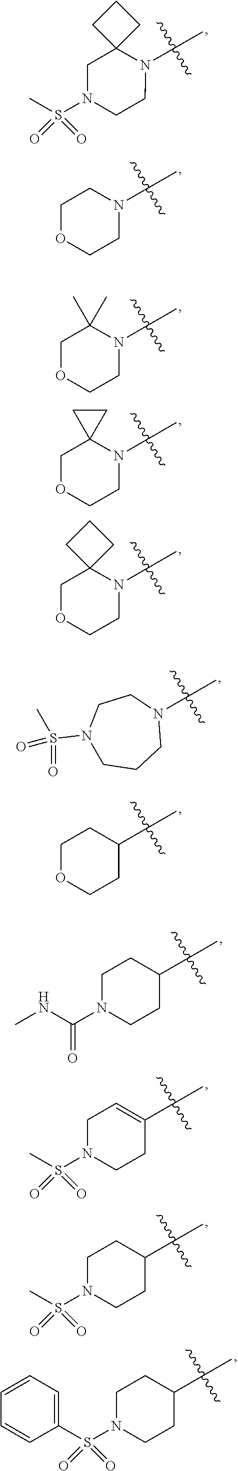

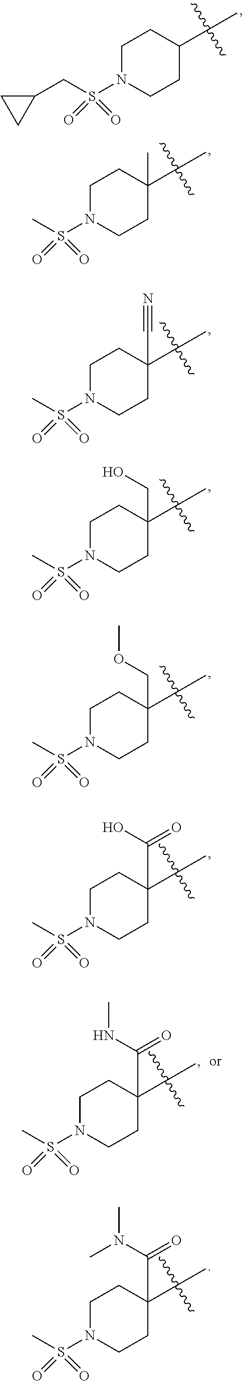

24. The compound of any one of claims 1-23, or a pharmaceutically acceptable salt or solvate thereof, wherein R¹ is

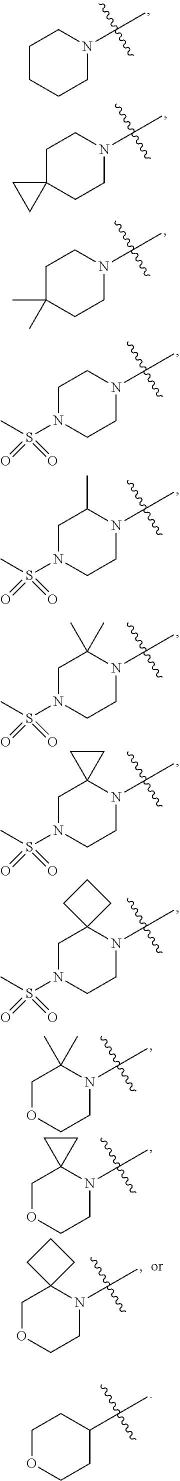

25. The compound of any one of claims 1-18, or a pharmaceutically acceptable salt or solvate thereof, wherein R¹ is C₃₋₈cycloalkyl optionally substituted with one, two, or three R⁶.

26. The compound of any one of claims 1-17, or a pharmaceutically acceptable salt or solvate thereof, wherein R¹ is selected from C₆₋₁₀aryl and C₁₋₉heteroaryl, wherein C₆₋₁₀aryl and C₁₋₉heteroaryl are substituted with one, two, or three R⁷.

27. The compound of claim 26, or a pharmaceutically acceptable salt or solvate thereof, wherein R¹ is C₁₋₉heteroaryl substituted with one, two, or three R⁷.

28. The compound of claim 27, or a pharmaceutically acceptable salt or solvate thereof, wherein R¹ is C₁₋₉heteroaryl selected from pyridinyl, pyrimidinyl, pyrazinyl, pyridazinyl, triazinyl, oxazolyl, thiazolyl, pyrazolyl, furanyl, thienyl, pyrrolyl, imidazolyl, triazolyl, tetrazolyl, isoxazolyl, isothiazolyl, oxadiazolyl, and thiadiazolyl, wherein pyridinyl, pyrimidinyl, pyrazinyl, pyridazinyl, triazinyl, oxazolyl, thiazolyl, pyrazolyl, furanyl, thienyl, pyrrolyl, imidazolyl, triazolyl, tetrazolyl, isoxazolyl, isothiazolyl, oxadiazolyl, and thiadiazolyl are substituted with one, two, or three R⁷.

29. The compound of any one of claims 26-28, or a pharmaceutically acceptable salt or solvate thereof, wherein R¹ is

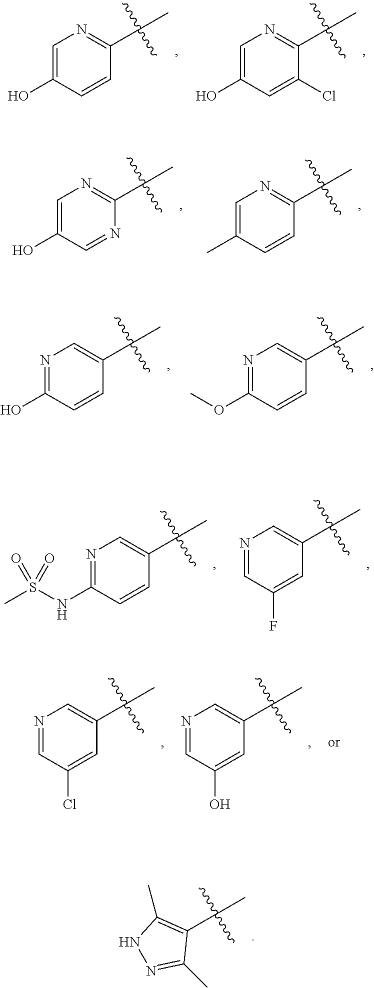

30. The compound of claim 26, or a pharmaceutically acceptable salt or solvate thereof, wherein R¹ is phenyl substituted with one, two, or three R⁷.

31. The compound of any one of claims 1-30, or a pharmaceutically acceptable salt or solvate thereof, wherein each R⁵ is independently selected from H, halogen, C₁₋₆alkyl, and —OR¹⁰.

32. The compound of any one of claims 1-31, or a pharmaceutically acceptable salt or solvate thereof, wherein each R⁵ is H.

33. The compound of any one of claims 1-32, or a pharmaceutically acceptable salt or solvate thereof, wherein each R⁴ is independently selected from H, halogen, C₁₋₆alkyl, and C₃₋₆cycloalkyl.

34. The compound of any one of claims 1-33, or a pharmaceutically acceptable salt or solvate thereof, wherein each R³ is independently selected from H, halogen, C₁₋₆alkyl, C₁₋₆haloalkyl, and —OR¹⁰.

35. The compound of any one of claims 1-34, or a pharmaceutically acceptable salt or solvate thereof, wherein R² is H.

36. The compound of any one of claims 1-34, or a pharmaceutically acceptable salt or solvate thereof, wherein R² is halogen.

37. A compound selected from: 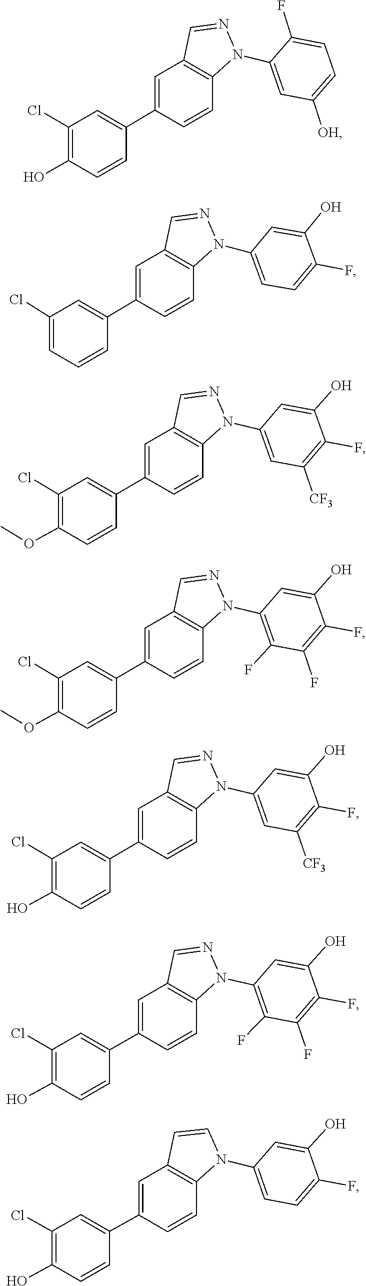 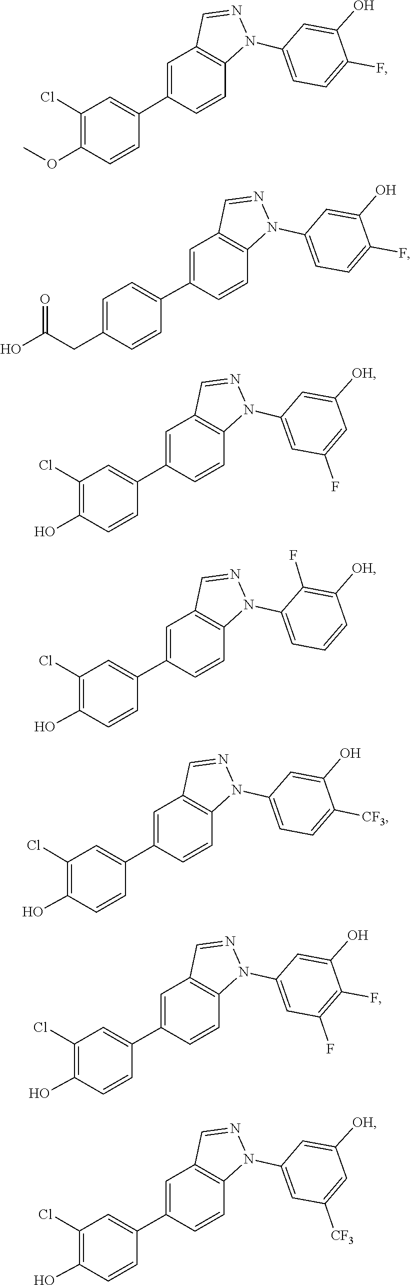 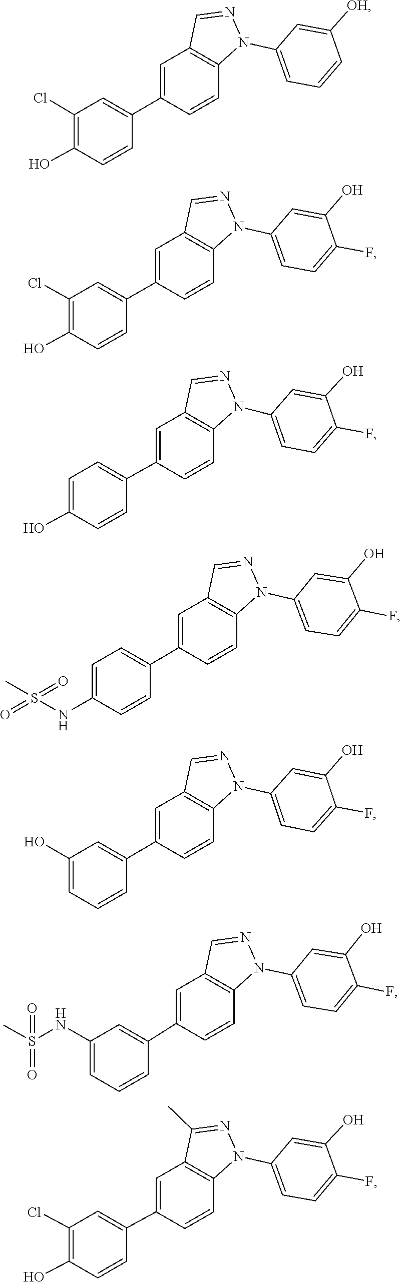 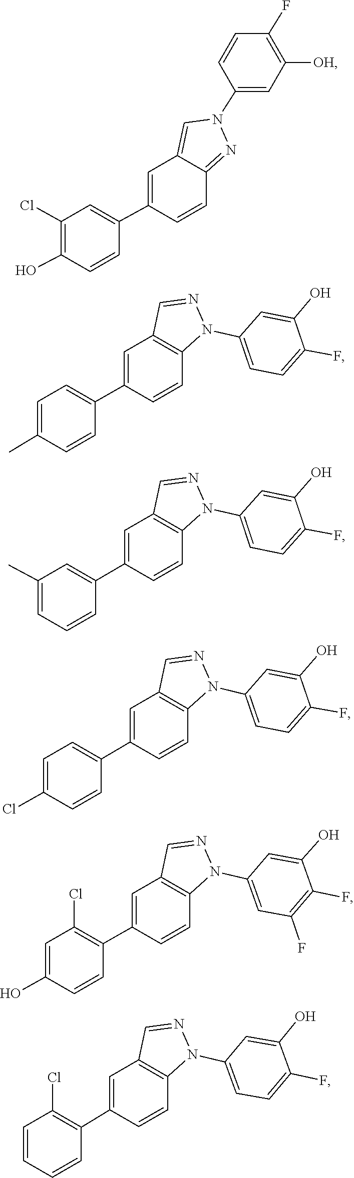 [SMILES: Oc1cc(-n2cc3cc(-c4ccc(O)c(Cl)c4)ccc3n2)ccc1F ; Cc1ccc(-c2ccc3c(cnn3-c3ccc(F)c(O)c3)c2)cc1 ; Cc1cccc(-c2ccc3c(cnn3-c3ccc(F)c(O)c3)c2)c1 ; Oc1cc(-n2ncc3cc(-c4ccc(Cl)cc4)ccc32)ccc1F ; Oc1ccc(-c2ccc3c(cnn3-c3cc(O)c(F)c(F)c3)c2)c(Cl)c1 ; Oc1cc(-n2ncc3cc(-c4ccccc4Cl)ccc32)ccc1F] 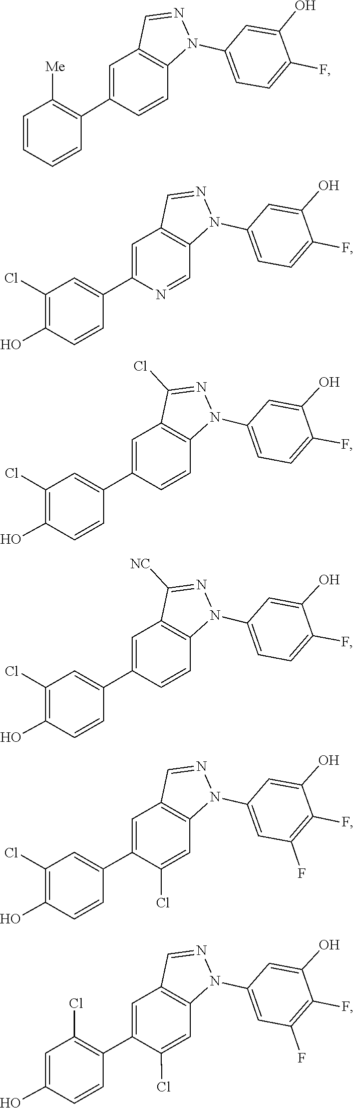 [SMILES: Cc1ccccc1-c1ccc2c(cnn2-c2ccc(F)c(O)c2)c1 ; Oc1cc(-n2ncc3cc(-c4ccc(O)c(Cl)c4)ncc32)ccc1F ; Oc1cc(-n2nc(Cl)c3cc(-c4ccc(O)c(Cl)c4)ccc32)ccc1F ; N#Cc1nn(-c2ccc(F)c(O)c2)c2ccc(-c3ccc(O)c(Cl)c3)cc12 ; Oc1ccc(-c2cc3cnn(-c4cc(O)c(F)c(F)c4)c3cc2Cl)cc1Cl ; Oc1ccc(-c2cc3cnn(-c4cc(O)c(F)c(F)c4)c3cc2Cl)c(Cl)c1] 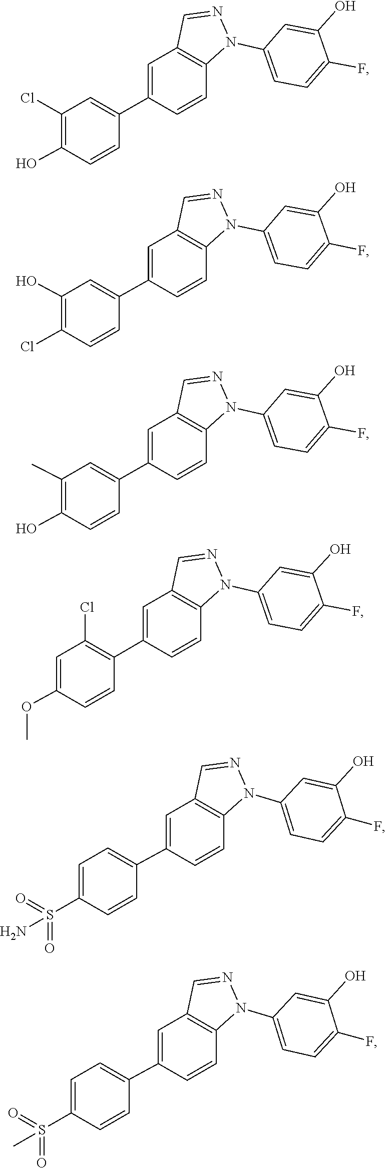 [SMILES: Oc1cc(-n2ncc3cc(-c4ccc(O)c(Cl)c4)ccc32)ccc1F ; Oc1cc(-n2ncc3cc(-c4ccc(Cl)c(O)c4)ccc32)ccc1F ; Cc1cc(-c2ccc3c(cnn3-c3ccc(F)c(O)c3)c2)ccc1O ; COc1ccc(-c2ccc3c(cnn3-c3ccc(F)c(O)c3)c2)c(Cl)c1 ; NS(=O)(=O)c1ccc(-c2ccc3c(cnn3-c3ccc(F)c(O)c3)c2)cc1 ; CS(=O)(=O)c1ccc(-c2ccc3c(cnn3-c3ccc(F)c(O)c3)c2)cc1] 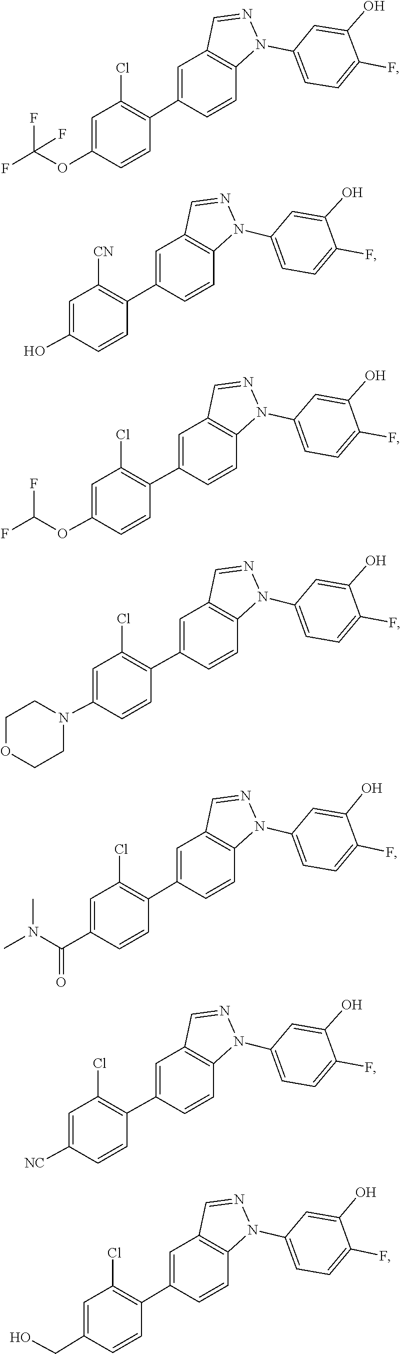 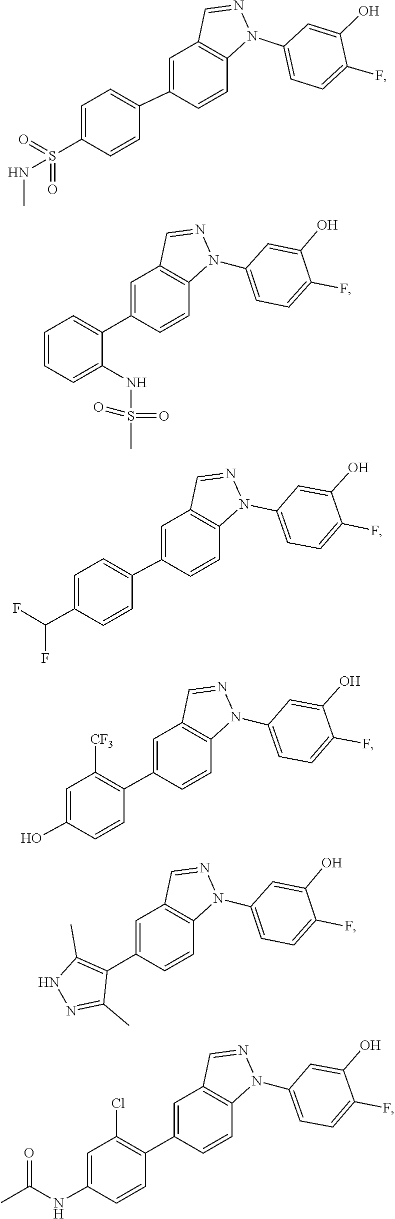 [SMILES: CNS(=O)(=O)c1ccc(-c2ccc3c(cnn3-c3ccc(F)c(O)c3)c2)cc1 ; CS(=O)(=O)Nc1ccccc1-c1ccc2c(cnn2-c2ccc(F)c(O)c2)c1 ; Oc1cc(-n2ncc3cc(-c4ccc(C(F)F)cc4)ccc32)ccc1F ; Oc1ccc(-c2ccc3c(cnn3-c3ccc(F)c(O)c3)c2)c(C(F)(F)F)c1 ; Cc1n[nH]c(C)c1-c1ccc2c(cnn2-c2ccc(F)c(O)c2)c1 ; CC(=O)Nc1ccc(-c2ccc3c(cnn3-c3ccc(F)c(O)c3)c2)c(Cl)c1] 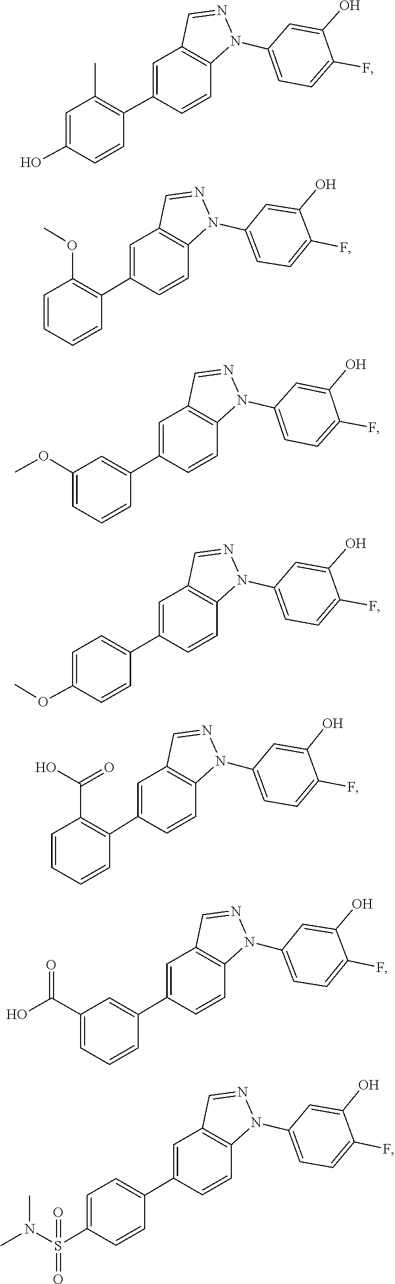 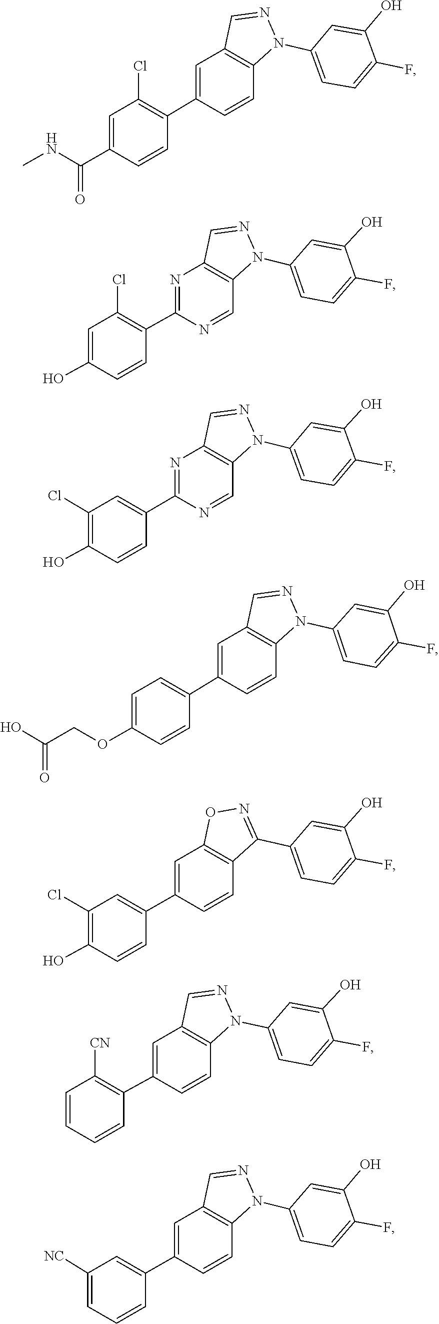 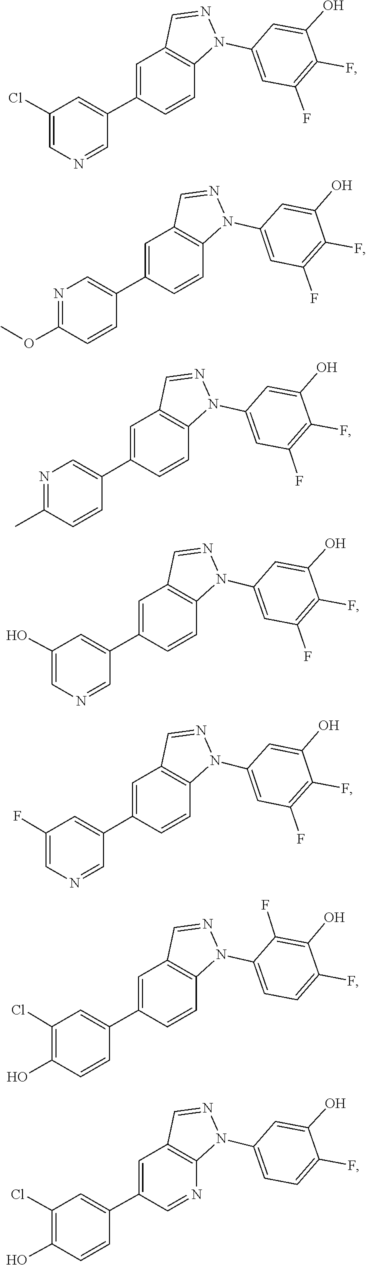 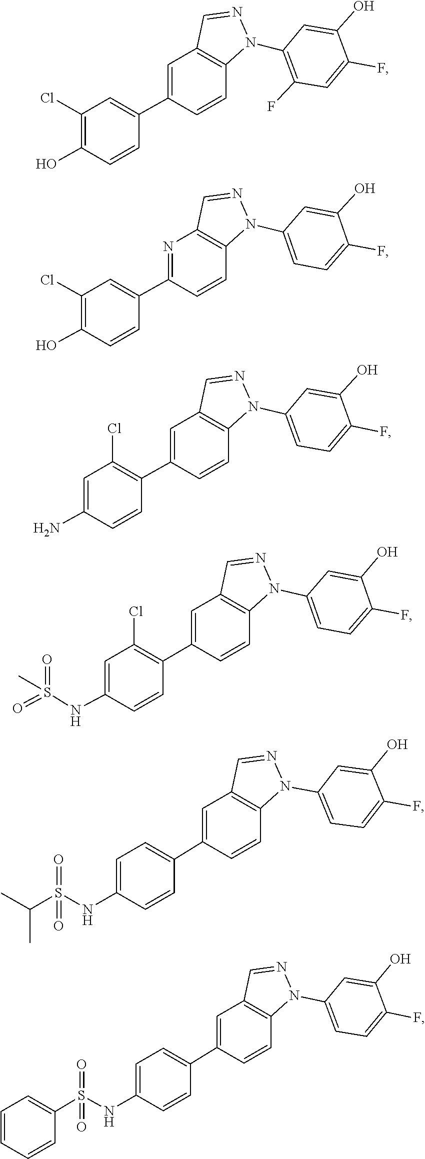 [SMILES: Oc1cc(-n2ncc3cc(-c4ccc(O)c(Cl)c4)ccc32)c(F)cc1F ; Oc1cc(-n2ncc3nc(-c4ccc(O)c(Cl)c4)ccc32)ccc1F ; Nc1ccc(-c2ccc3c(cnn3-c3ccc(F)c(O)c3)c2)c(Cl)c1 ; CS(=O)(=O)Nc1ccc(-c2ccc3c(cnn3-c3ccc(F)c(O)c3)c2)c(Cl)c1 ; CC(C)S(=O)(=O)Nc1ccc(-c2ccc3c(cnn3-c3ccc(F)c(O)c3)c2)cc1 ; O=S(=O)(Nc1ccc(-c2ccc3c(cnn3-c3ccc(F)c(O)c3)c2)cc1)c1ccccc1] 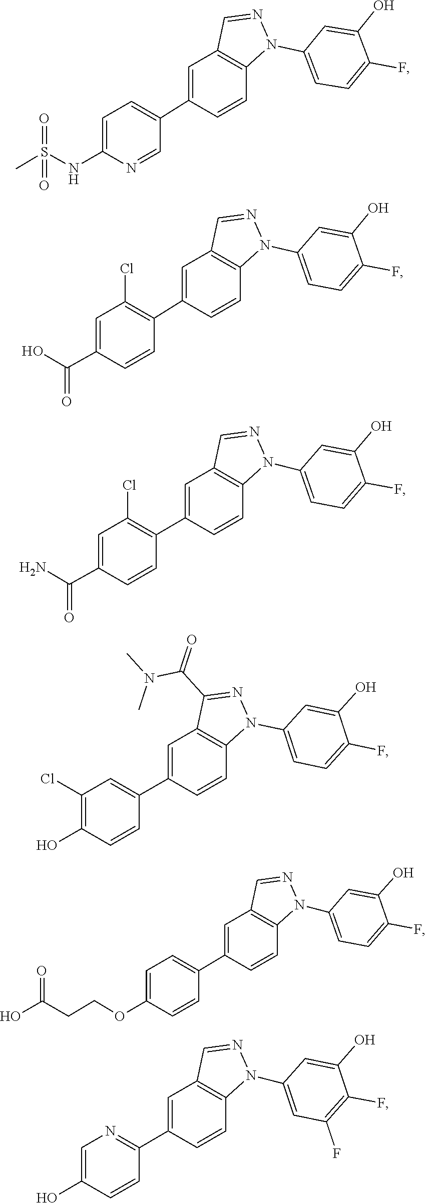 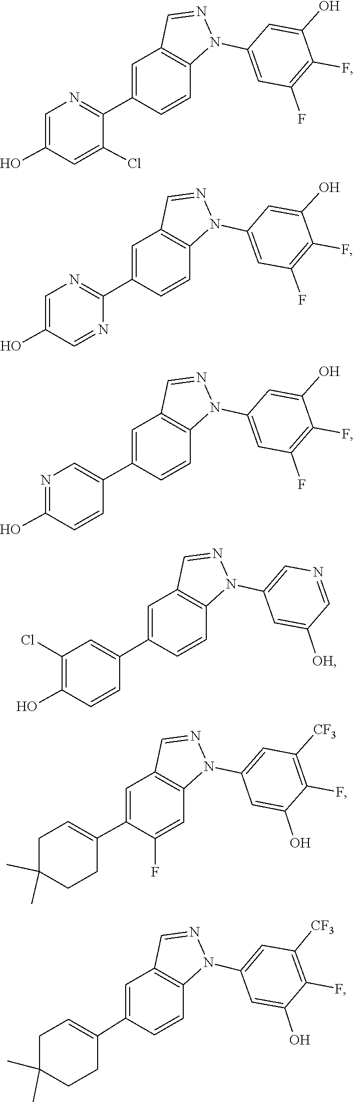 [SMILES: Oc1cnc(-c2ccc3c(cnn3-c3cc(O)c(F)c(F)c3)c2)c(Cl)c1 ; Oc1cnc(-c2ccc3c(cnn3-c3cc(O)c(F)c(F)c3)c2)nc1 ; Oc1ccc(-c2ccc3c(cnn3-c3cc(O)c(F)c(F)c3)c2)cn1 ; Oc1cncc(-n2ncc3cc(-c4ccc(O)c(Cl)c4)ccc32)c1 ; CC1(C)CC=C(c2cc3cnn(-c4cc(O)c(F)c(C(F)(F)F)c4)c3cc2F)CC1 ; CC1(C)CC=C(c2ccc3c(cnn3-c3cc(O)c(F)c(C(F)(F)F)c3)c2)CC1] 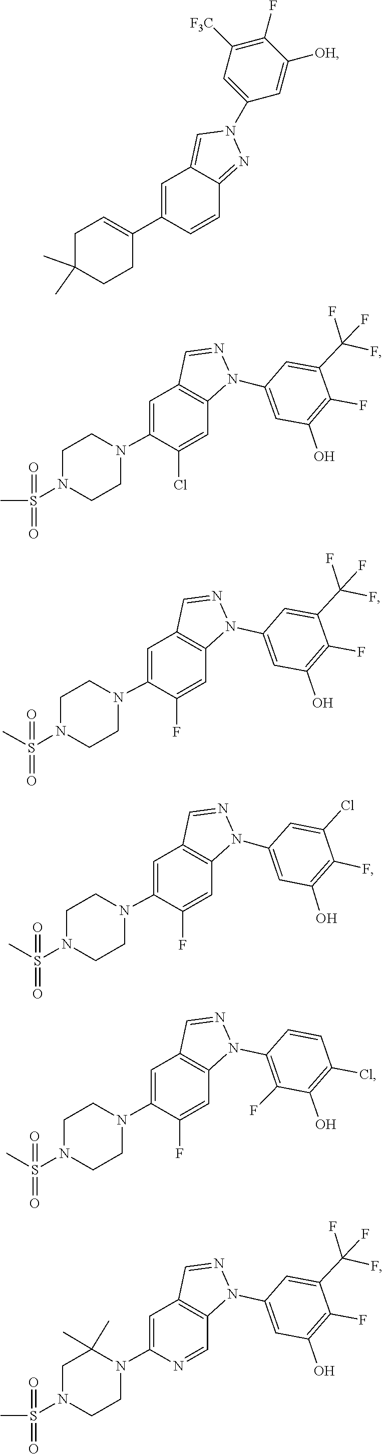 [SMILES: CC1(C)CC=C(c2ccc3nn(-c4cc(O)c(F)c(C(F)(F)F)c4)cc3c2)CC1 ; CS(=O)(=O)N1CCN(c2cc3cnn(-c4cc(O)c(F)c(C(F)(F)F)c4)c3cc2Cl)CC1 ; CS(=O)(=O)N1CCN(c2cc3cnn(-c4cc(O)c(F)c(C(F)(F)F)c4)c3cc2F)CC1 ; CS(=O)(=O)N1CCN(c2cc3cnn(-c4cc(O)c(F)c(Cl)c4)c3cc2F)CC1 ; CS(=O)(=O)N1CCN(c2cc3cnn(-c4ccc(Cl)c(O)c4F)c3cc2F)CC1 ; CC1(C)CN(S(C)(=O)=O)CCN1c1cc2cnn(-c3cc(O)c(F)c(C(F)(F)F)c3)c2cn1] 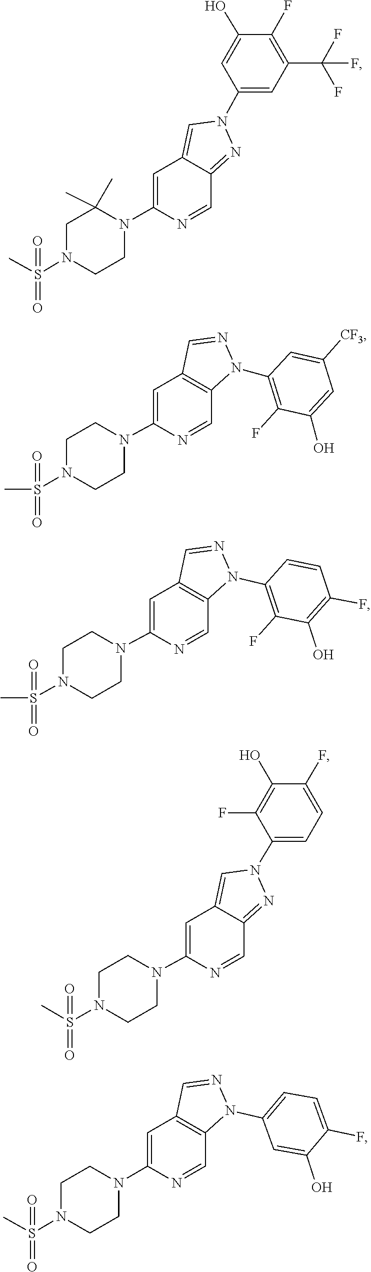 [SMILES: CC1(C)CN(S(C)(=O)=O)CCN1c1cc2cn(-c3cc(O)c(F)c(C(F)(F)F)c3)nc2cn1 ; CS(=O)(=O)N1CCN(c2cc3cnn(-c4cc(C(F)(F)F)cc(O)c4F)c3cn2)CC1 ; CS(=O)(=O)N1CCN(c2cc3cnn(-c4ccc(F)c(O)c4F)c3cn2)CC1 ; CS(=O)(=O)N1CCN(c2cc3cn(-c4ccc(F)c(O)c4F)nc3cn2)CC1 ; CS(=O)(=O)N1CCN(c2cc3cnn(-c4ccc(F)c(O)c4)c3cn2)CC1] 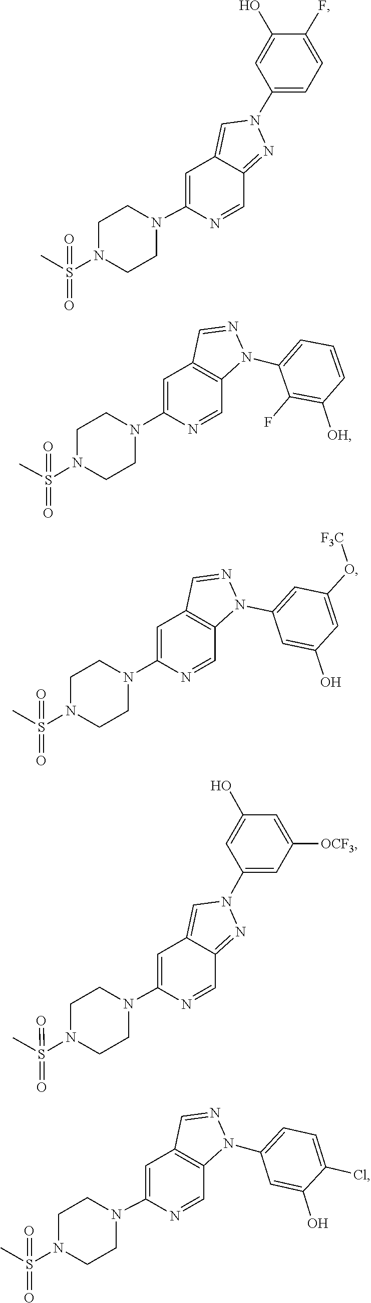 [SMILES: CS(=O)(=O)N1CCN(c2cc3cn(-c4ccc(F)c(O)c4)nc3cn2)CC1 ; CS(=O)(=O)N1CCN(c2cc3cnn(-c4cccc(O)c4F)c3cn2)CC1 ; CS(=O)(=O)N1CCN(c2cc3cnn(-c4cc(O)cc(OC(F)(F)F)c4)c3cn2)CC1 ; CS(=O)(=O)N1CCN(c2cc3cn(-c4cc(O)cc(OC(F)(F)F)c4)nc3cn2)CC1 ; CS(=O)(=O)N1CCN(c2cc3cnn(-c4ccc(Cl)c(O)c4)c3cn2)CC1] 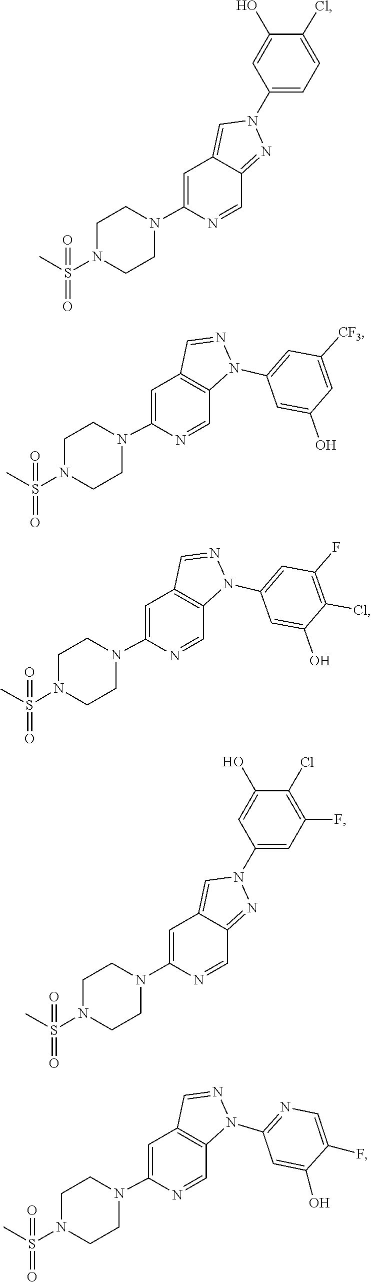 [SMILES: CS(=O)(=O)N1CCN(c2cc3cn(-c4ccc(Cl)c(O)c4)nc3cn2)CC1 ; CS(=O)(=O)N1CCN(c2cc3cnn(-c4cc(O)cc(C(F)(F)F)c4)c3cn2)CC1 ; CS(=O)(=O)N1CCN(c2cc3cnn(-c4cc(O)c(Cl)c(F)c4)c3cn2)CC1 ; CS(=O)(=O)N1CCN(c2cc3cn(-c4cc(O)c(Cl)c(F)c4)nc3cn2)CC1 ; CS(=O)(=O)N1CCN(c2cc3cnn(-c4cc(O)c(F)cn4)c3cn2)CC1] 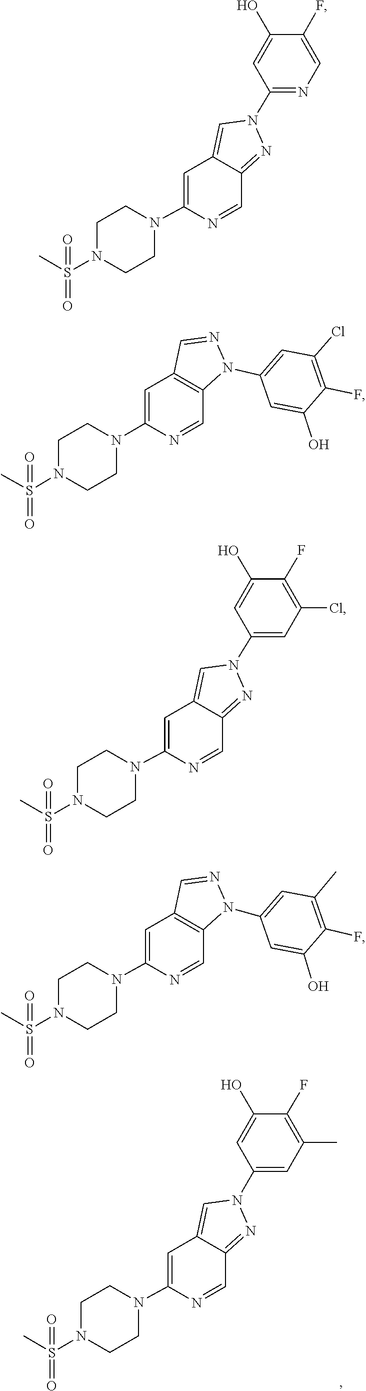 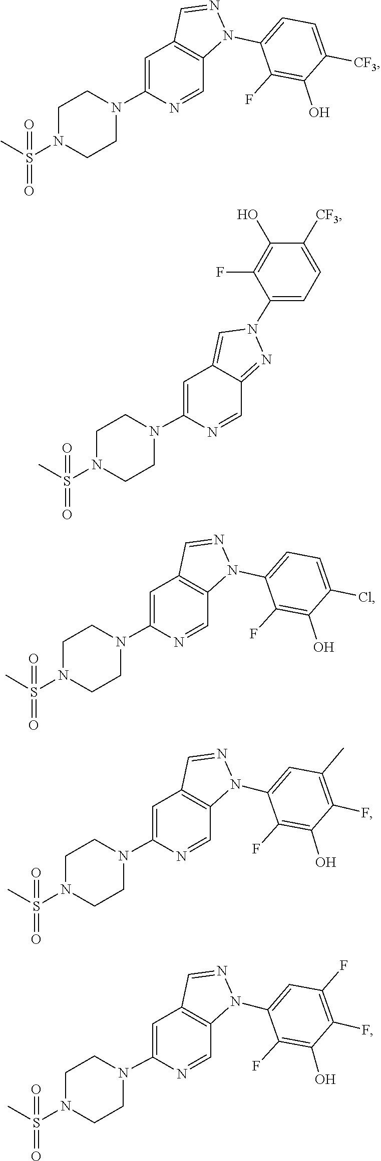 [SMILES: CS(=O)(=O)N1CCN(c2cc3cnn(-c4ccc(C(F)(F)F)c(O)c4F)c3cn2)CC1 ; CS(=O)(=O)N1CCN(c2cc3cn(-c4ccc(C(F)(F)F)c(O)c4F)nc3cn2)CC1 ; CS(=O)(=O)N1CCN(c2cc3cnn(-c4ccc(Cl)c(O)c4F)c3cn2)CC1 ; Cc1cc(-n2ncc3cc(N4CCN(S(C)(=O)=O)CC4)ncc32)c(F)c(O)c1F ; CS(=O)(=O)N1CCN(c2cc3cnn(-c4cc(F)c(F)c(O)c4F)c3cn2)CC1] 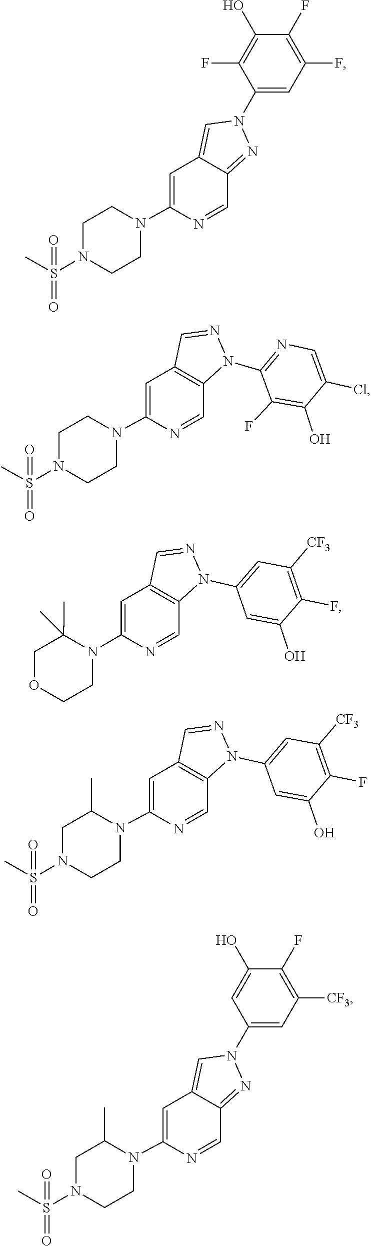 [SMILES: CS(=O)(=O)N1CCN(c2cc3cn(-c4cc(F)c(F)c(O)c4F)nc3cn2)CC1 ; CS(=O)(=O)N1CCN(c2cc3cnn(-c4ncc(Cl)c(O)c4F)c3cn2)CC1 ; CC1(C)COCCN1c1cc2cnn(-c3cc(O)c(F)c(C(F)(F)F)c3)c2cn1 ; CC1CN(S(C)(=O)=O)CCN1c1cc2cnn(-c3cc(O)c(F)c(C(F)(F)F)c3)c2cn1 ; CC1CN(S(C)(=O)=O)CCN1c1cc2cn(-c3cc(O)c(F)c(C(F)(F)F)c3)nc2cn1] 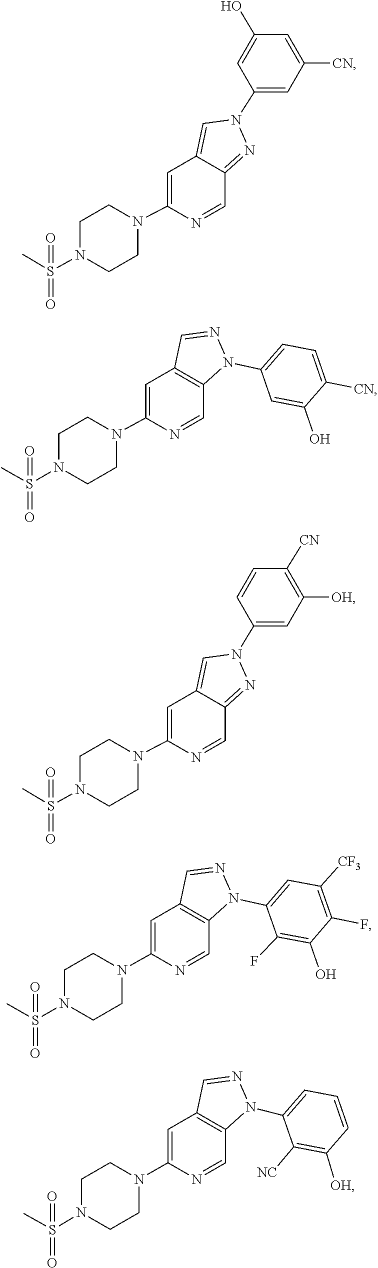 [SMILES: CS(=O)(=O)N1CCN(c2cc3cn(-c4cc(O)cc(C#N)c4)nc3cn2)CC1 ; CS(=O)(=O)N1CCN(c2cc3cnn(-c4ccc(C#N)c(O)c4)c3cn2)CC1 ; CS(=O)(=O)N1CCN(c2cc3cn(-c4ccc(C#N)c(O)c4)nc3cn2)CC1 ; CS(=O)(=O)N1CCN(c2cc3cnn(-c4cc(C(F)(F)F)c(F)c(O)c4F)c3cn2)CC1 ; CS(=O)(=O)N1CCN(c2cc3cnn(-c4cccc(O)c4C#N)c3cn2)CC1] 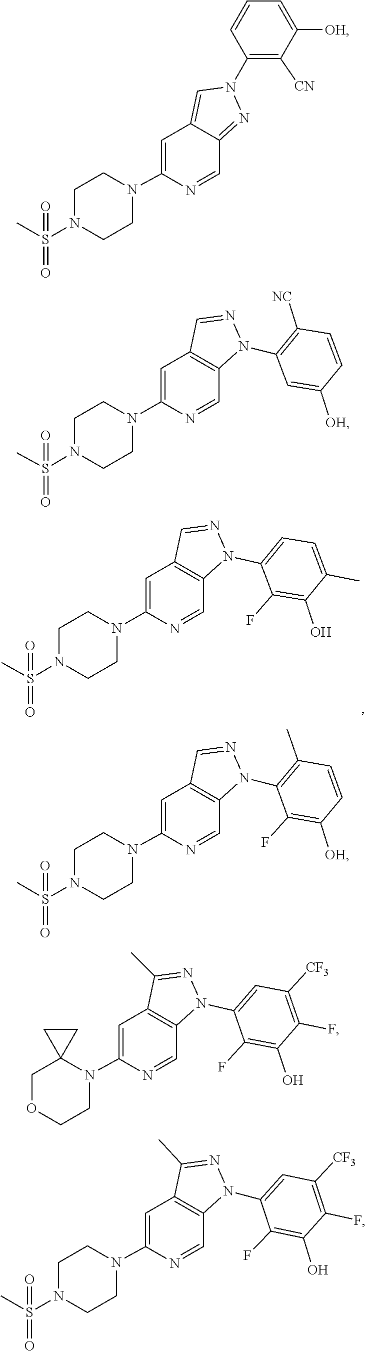 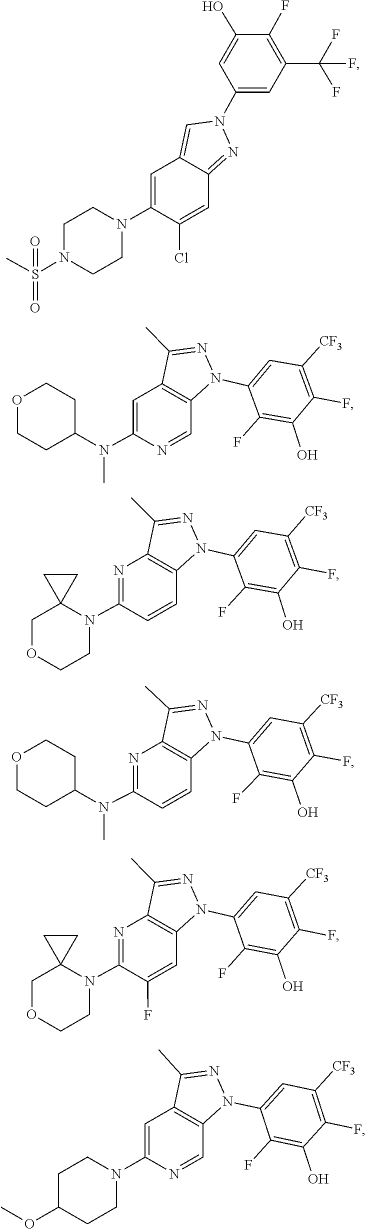 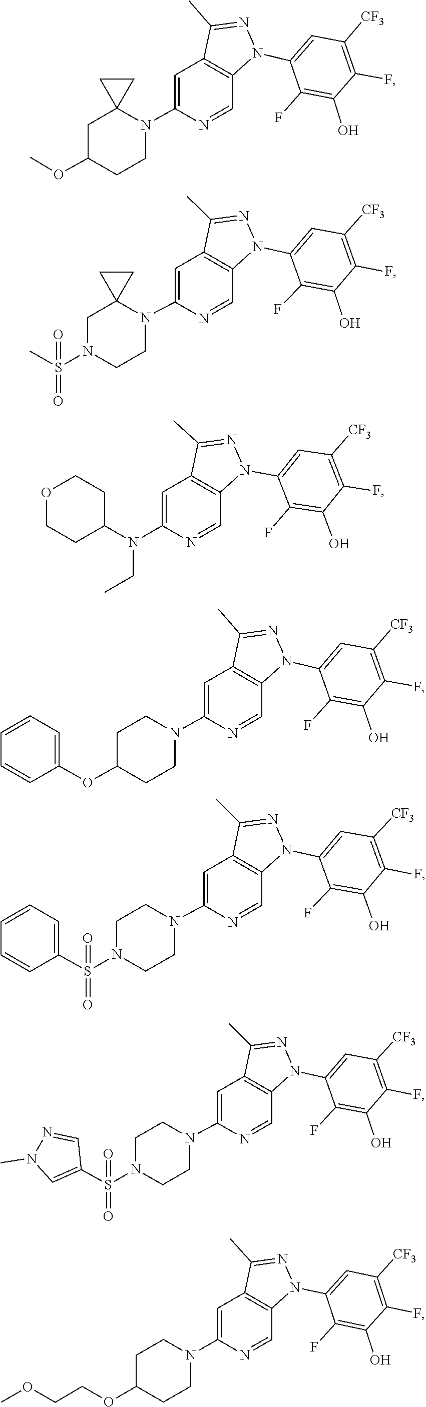 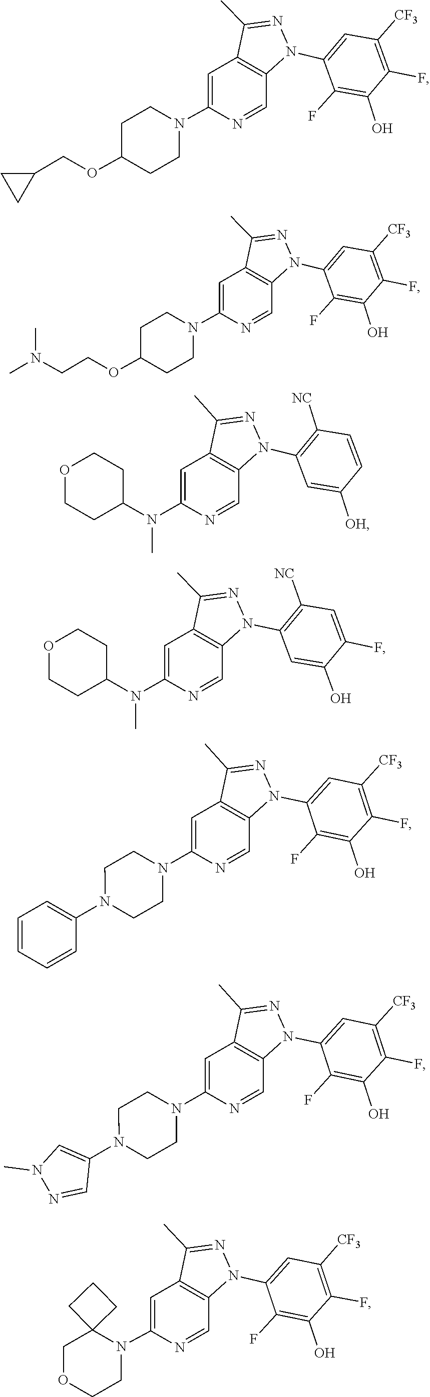 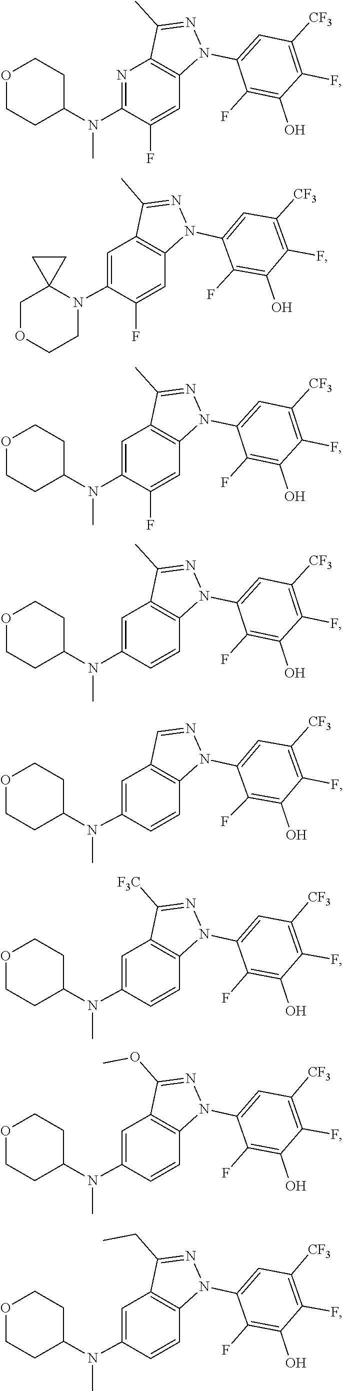 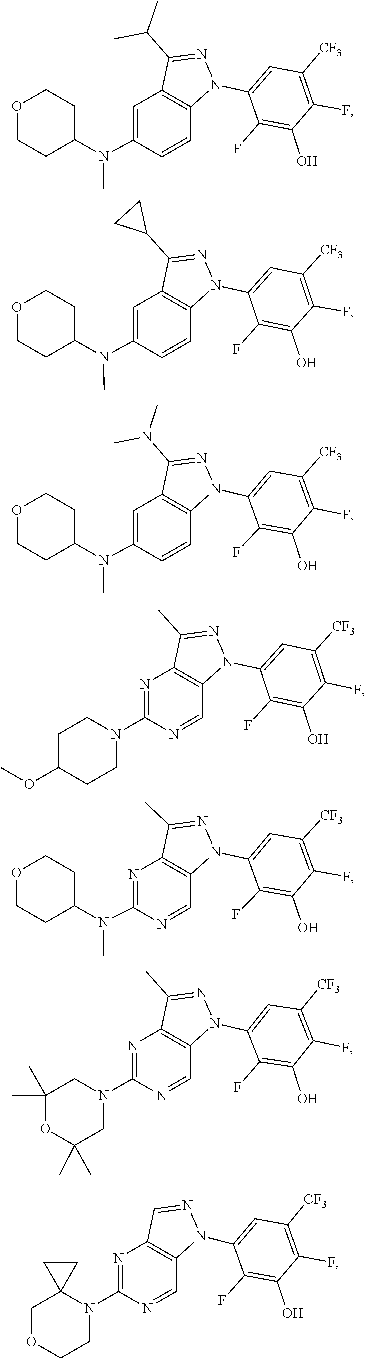 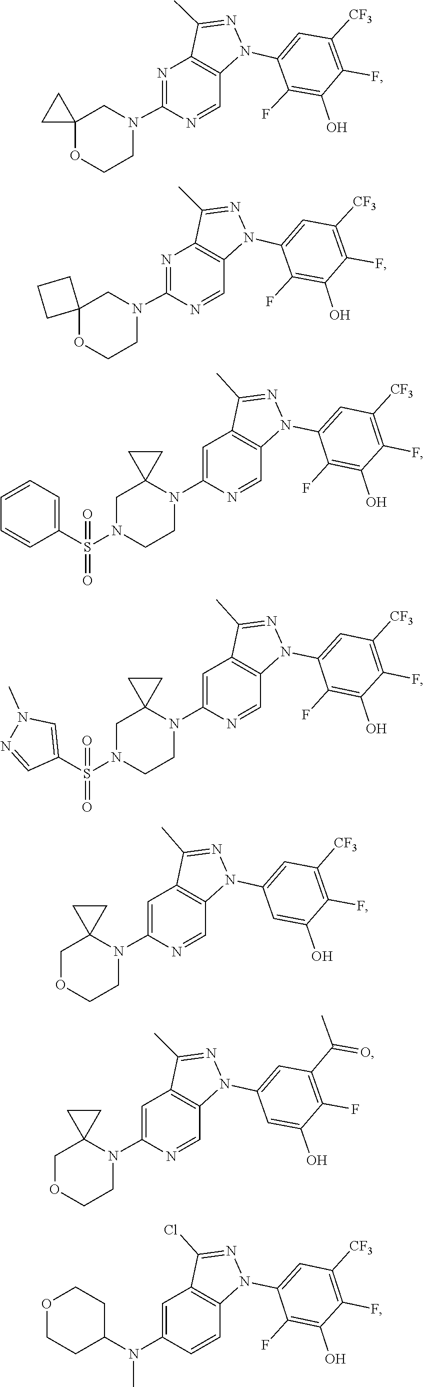 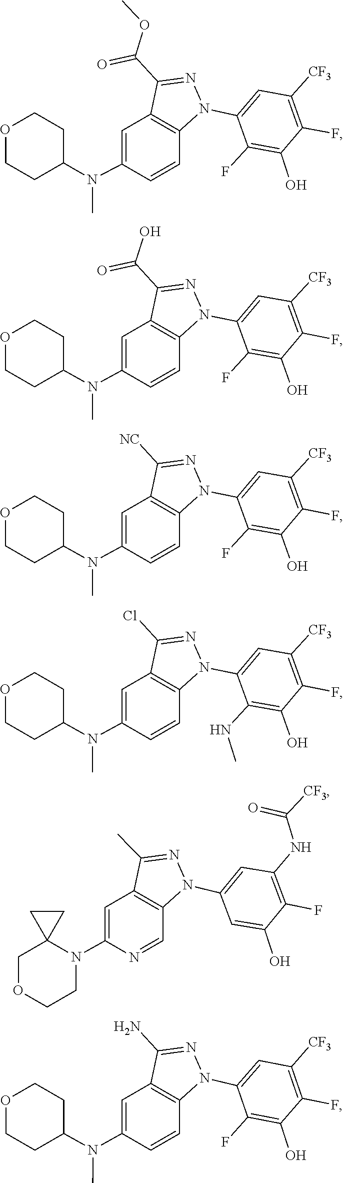 [SMILES: COC(=O)c1nn(-c2cc(C(F)(F)F)c(F)c(O)c2F)c2ccc(N(C)C3CCOCC3)cc12 ; CN(c1ccc2c(c1)c(C(=O)O)nn2-c1cc(C(F)(F)F)c(F)c(O)c1F)C1CCOCC1 ; CN(c1ccc2c(c1)c(C#N)nn2-c1cc(C(F)(F)F)c(F)c(O)c1F)C1CCOCC1 ; CNc1c(-n2nc(Cl)c3cc(N(C)C4CCOCC4)ccc32)cc(C(F)(F)F)c(F)c1O ; Cc1nn(-c2cc(O)c(F)c(NC(=O)C(F)(F)F)c2)c2cnc(N3CCOCC34CC4)cc12 ; CN(c1ccc2c(c1)c(N)nn2-c1cc(C(F)(F)F)c(F)c(O)c1F)C1CCOCC1] 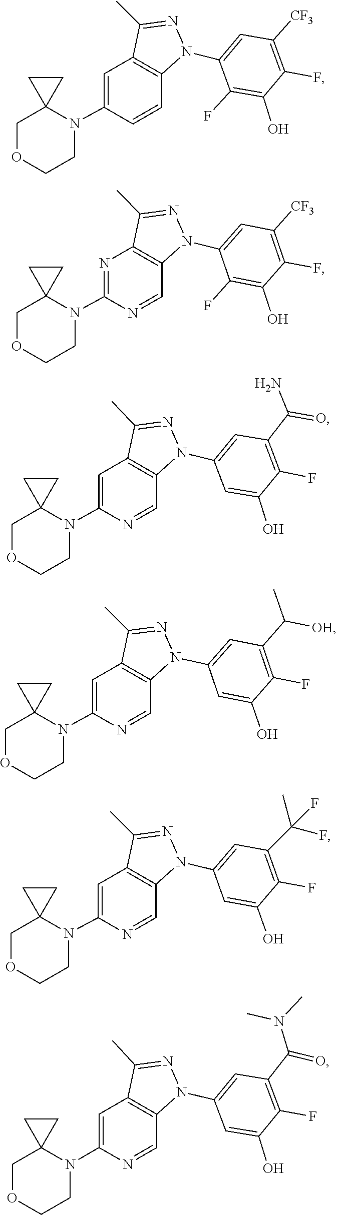 [SMILES: Cc1nn(-c2cc(C(F)(F)F)c(F)c(O)c2F)c2ccc(N3CCOCC34CC4)cc12 ; Cc1nn(-c2cc(C(F)(F)F)c(F)c(O)c2F)c2cnc(N3CCOCC34CC4)nc12 ; Cc1nn(-c2cc(O)c(F)c(C(N)=O)c2)c2cnc(N3CCOCC34CC4)cc12 ; Cc1nn(-c2cc(O)c(F)c(C(C)O)c2)c2cnc(N3CCOCC34CC4)cc12 ; Cc1nn(-c2cc(O)c(F)c(C(C)(F)F)c2)c2cnc(N3CCOCC34CC4)cc12 ; Cc1nn(-c2cc(O)c(F)c(C(=O)N(C)C)c2)c2cnc(N3CCOCC34CC4)cc12] 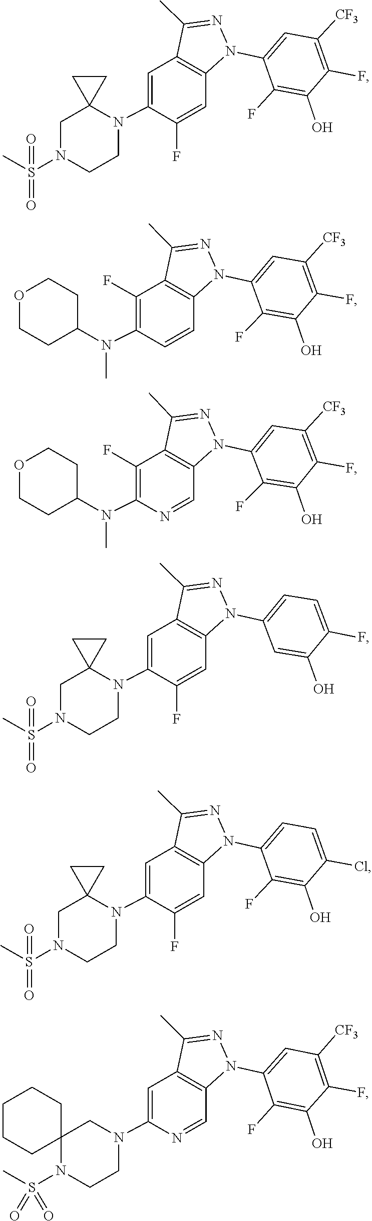 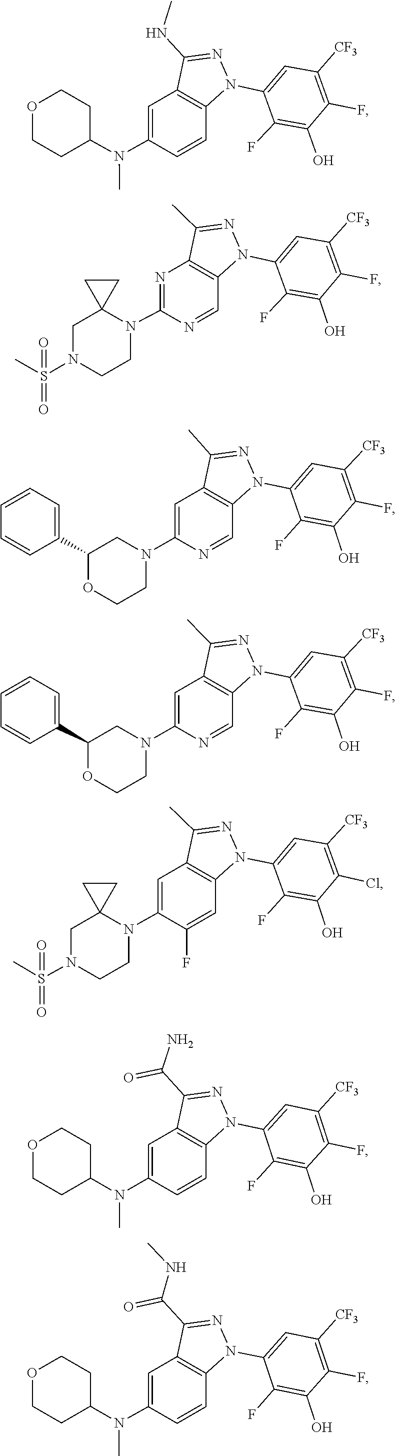 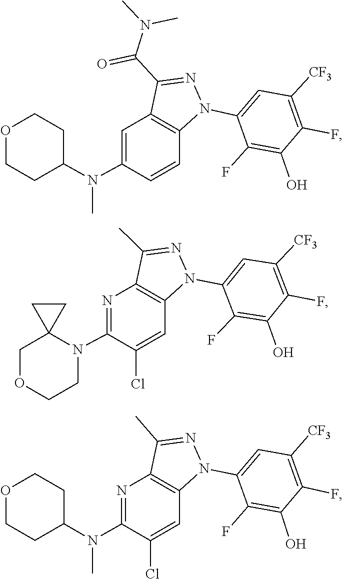 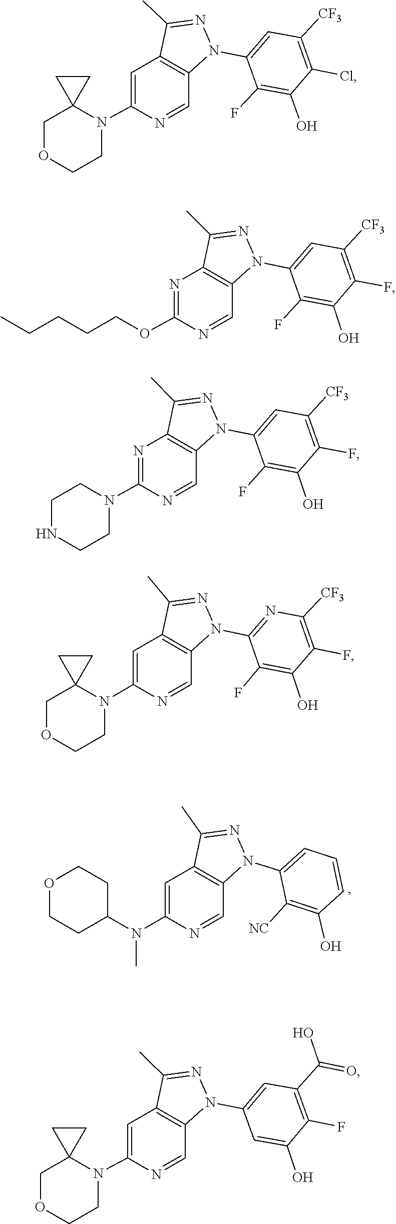 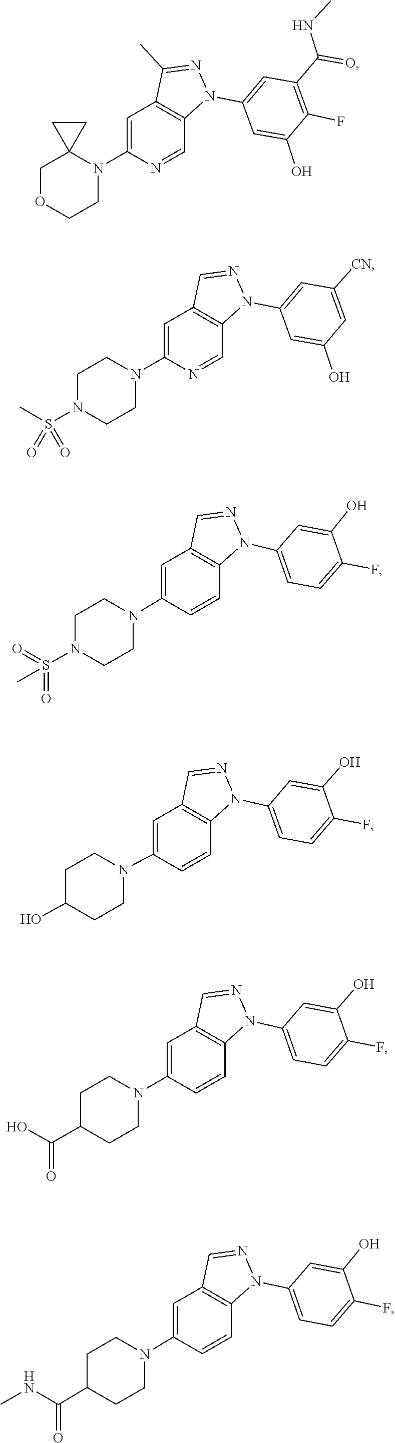 [SMILES: CNC(=O)c1cc(-n2nc(C)c3cc(N4CCOCC45CC5)ncc32)cc(O)c1F ; CS(=O)(=O)N1CCN(c2cc3cnn(-c4cc(O)cc(C#N)c4)c3cn2)CC1 ; CS(=O)(=O)N1CCN(c2ccc3c(cnn3-c3ccc(F)c(O)c3)c2)CC1 ; Oc1cc(-n2ncc3cc(N4CCC(O)CC4)ccc32)ccc1F ; O=C(O)C1CCN(c2ccc3c(cnn3-c3ccc(F)c(O)c3)c2)CC1 ; CNC(=O)C1CCN(c2ccc3c(cnn3-c3ccc(F)c(O)c3)c2)CC1] 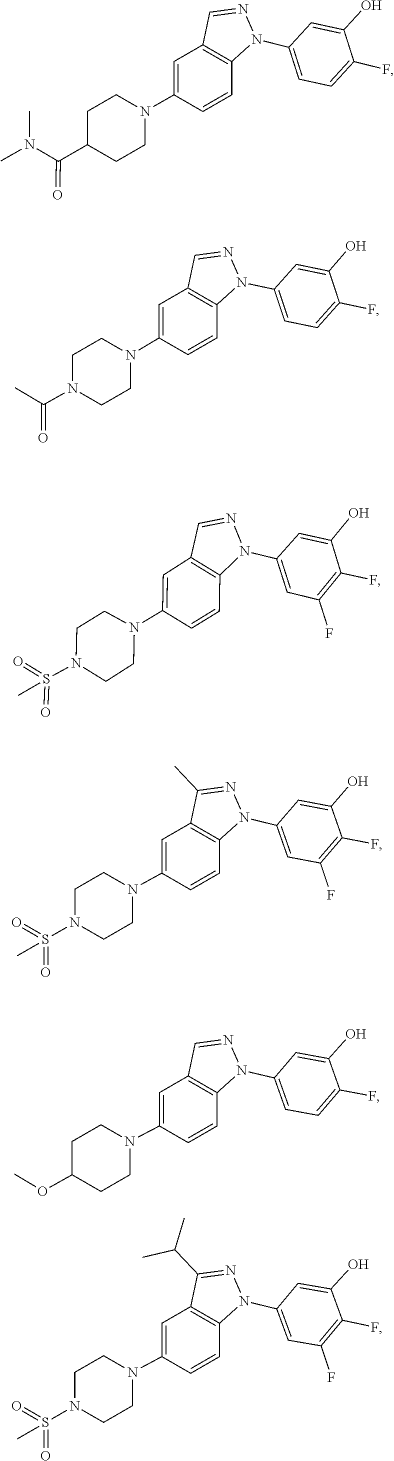 [SMILES: CN(C)C(=O)C1CCN(c2ccc3c(cnn3-c3ccc(F)c(O)c3)c2)CC1 ; CC(=O)N1CCN(c2ccc3c(cnn3-c3ccc(F)c(O)c3)c2)CC1 ; CS(=O)(=O)N1CCN(c2ccc3c(cnn3-c3cc(O)c(F)c(F)c3)c2)CC1 ; Cc1nn(-c2cc(O)c(F)c(F)c2)c2ccc(N3CCN(S(C)(=O)=O)CC3)cc12 ; COC1CCN(c2ccc3c(cnn3-c3ccc(F)c(O)c3)c2)CC1 ; CC(C)c1nn(-c2cc(O)c(F)c(F)c2)c2ccc(N3CCN(S(C)(=O)=O)CC3)cc12] 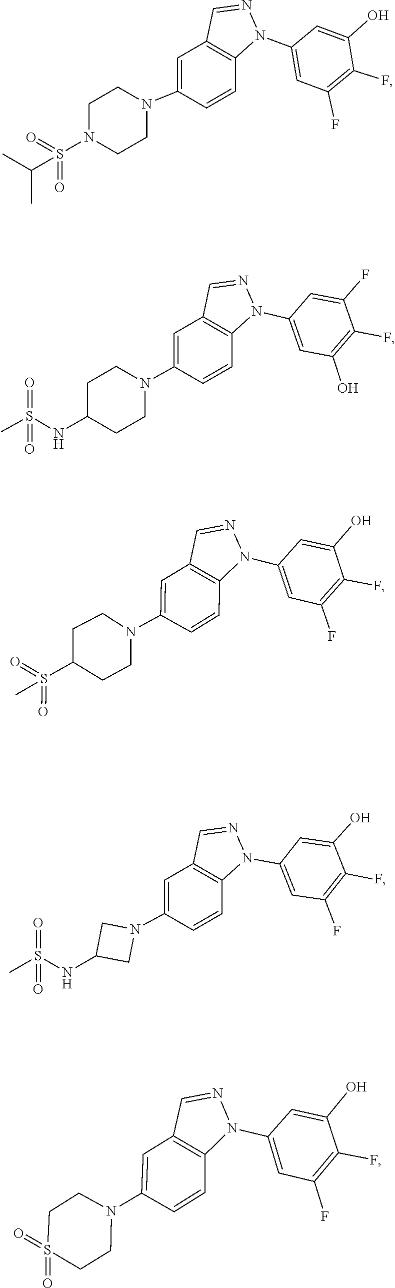 [SMILES: CC(C)S(=O)(=O)N1CCN(c2ccc3c(cnn3-c3cc(O)c(F)c(F)c3)c2)CC1 ; CS(=O)(=O)NC1CCN(c2ccc3c(cnn3-c3cc(O)c(F)c(F)c3)c2)CC1 ; CS(=O)(=O)C1CCN(c2ccc3c(cnn3-c3cc(O)c(F)c(F)c3)c2)CC1 ; CS(=O)(=O)NC1CN(c2ccc3c(cnn3-c3cc(O)c(F)c(F)c3)c2)C1 ; O=S1(=O)CCN(c2ccc3c(cnn3-c3cc(O)c(F)c(F)c3)c2)CC1] 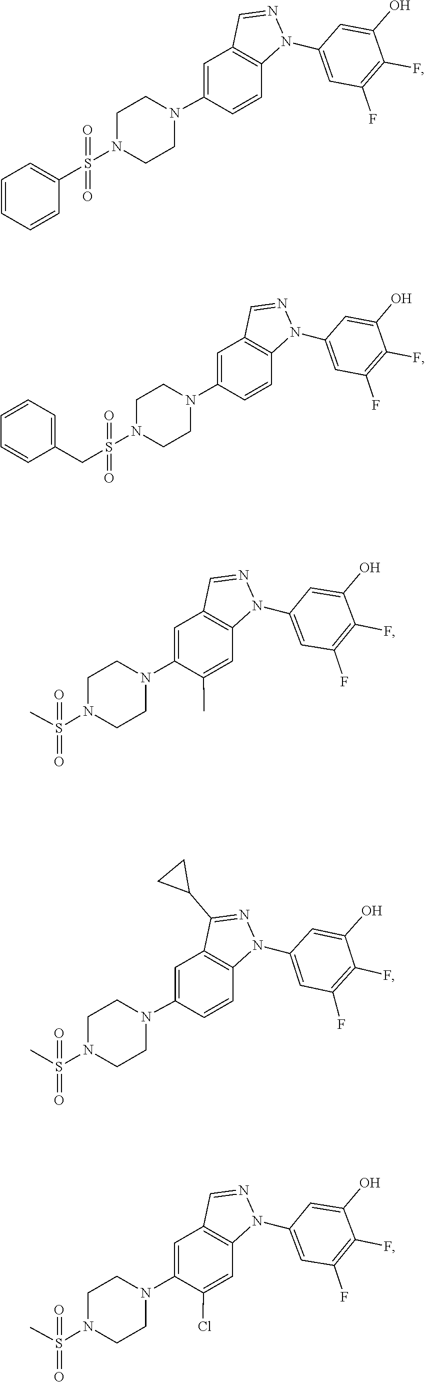 [SMILES: O=S(=O)(c1ccccc1)N1CCN(c2ccc3c(cnn3-c3cc(O)c(F)c(F)c3)c2)CC1 ; O=S(=O)(Cc1ccccc1)N1CCN(c2ccc3c(cnn3-c3cc(O)c(F)c(F)c3)c2)CC1 ; Cc1cc2c(cnn2-c2cc(O)c(F)c(F)c2)cc1N1CCN(S(C)(=O)=O)CC1 ; CS(=O)(=O)N1CCN(c2ccc3c(c2)c(C2CC2)nn3-c2cc(O)c(F)c(F)c2)CC1 ; CS(=O)(=O)N1CCN(c2cc3cnn(-c4cc(O)c(F)c(F)c4)c3cc2Cl)CC1] 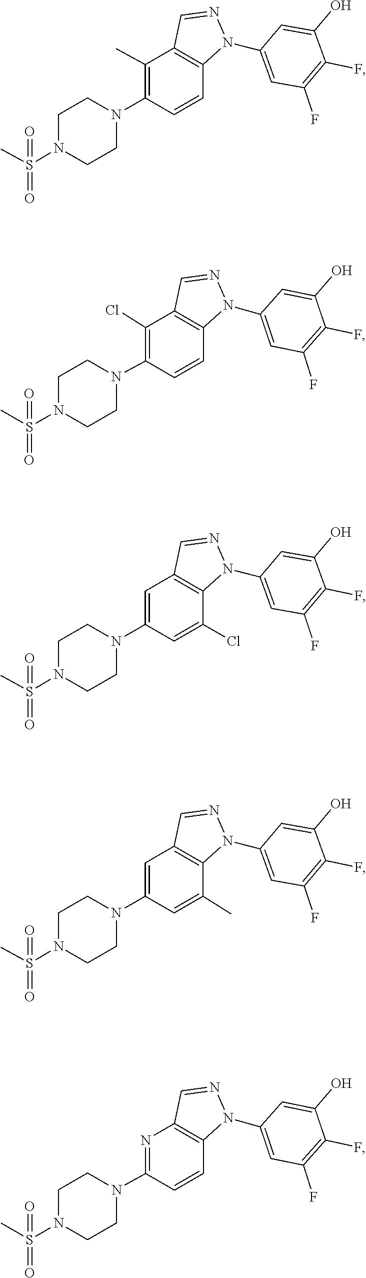 [SMILES: Cc1c(N2CCN(S(C)(=O)=O)CC2)ccc2c1cnn2-c1cc(O)c(F)c(F)c1 ; CS(=O)(=O)N1CCN(c2ccc3c(cnn3-c3cc(O)c(F)c(F)c3)c2Cl)CC1 ; CS(=O)(=O)N1CCN(c2cc(Cl)c3c(cnn3-c3cc(O)c(F)c(F)c3)c2)CC1 ; Cc1cc(N2CCN(S(C)(=O)=O)CC2)cc2cnn(-c3cc(O)c(F)c(F)c3)c12 ; CS(=O)(=O)N1CCN(c2ccc3c(cnn3-c3cc(O)c(F)c(F)c3)n2)CC1] 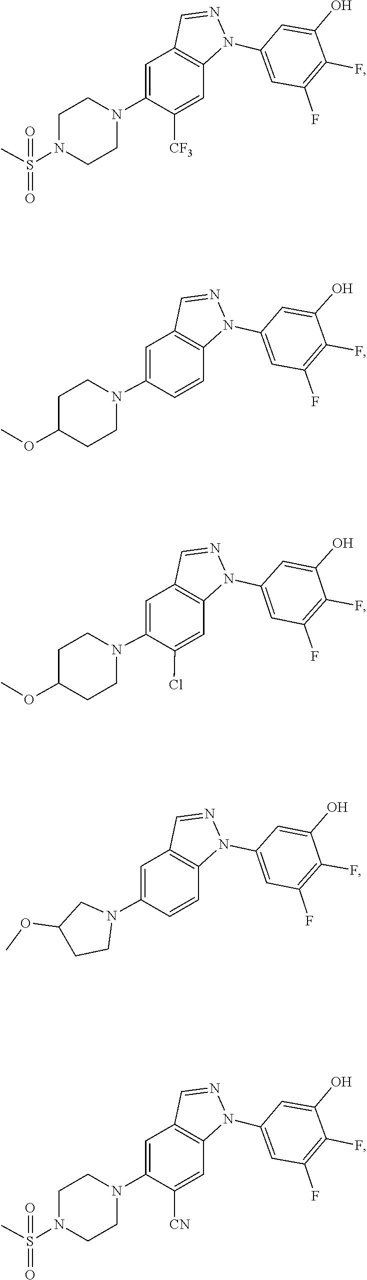 [SMILES: CS(=O)(=O)N1CCN(c2cc3cnn(-c4cc(O)c(F)c(F)c4)c3cc2C(F)(F)F)CC1 ; COC1CCN(c2ccc3c(cnn3-c3cc(O)c(F)c(F)c3)c2)CC1 ; COC1CCN(c2cc3cnn(-c4cc(O)c(F)c(F)c4)c3cc2Cl)CC1 ; COC1CCN(c2ccc3c(cnn3-c3cc(O)c(F)c(F)c3)c2)C1 ; CS(=O)(=O)N1CCN(c2cc3cnn(-c4cc(O)c(F)c(F)c4)c3cc2C#N)CC1] 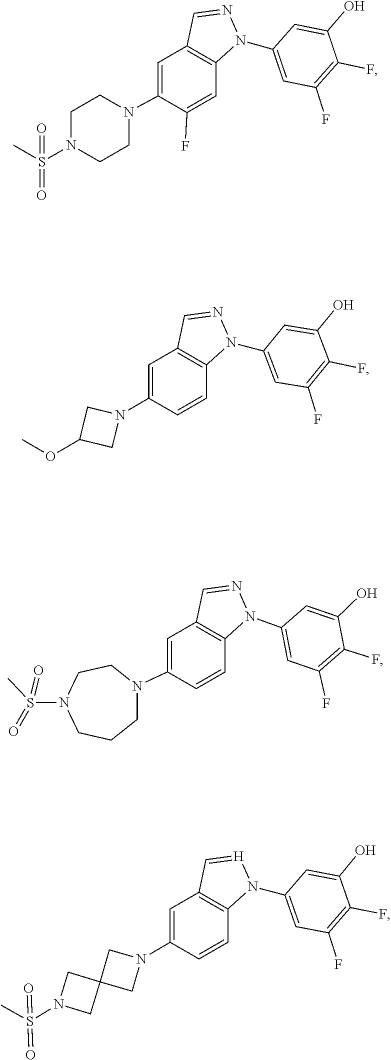 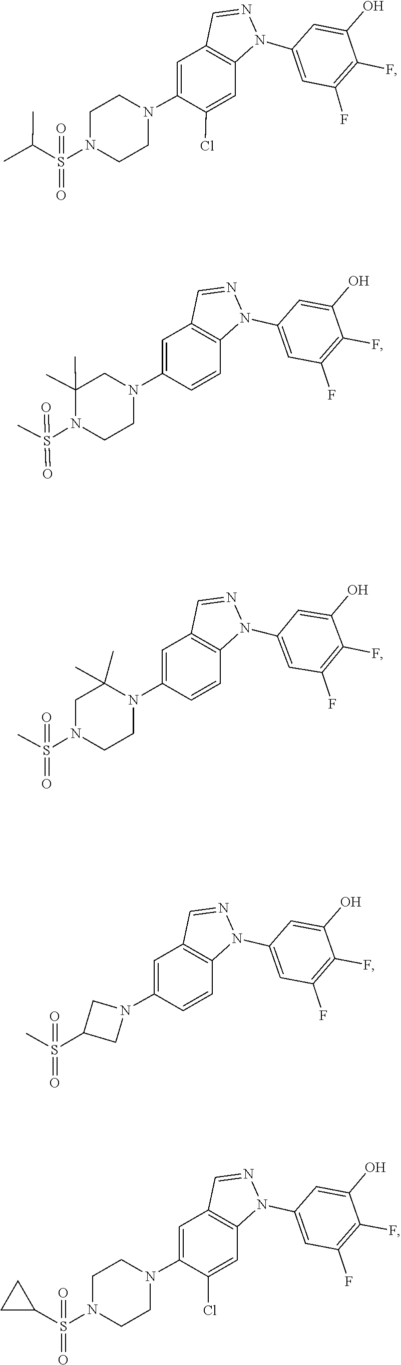 [SMILES: CC(C)S(=O)(=O)N1CCN(c2cc3cnn(-c4cc(O)c(F)c(F)c4)c3cc2Cl)CC1 ; CC1(C)CN(c2ccc3c(cnn3-c3cc(O)c(F)c(F)c3)c2)CCN1S(C)(=O)=O ; CC1(C)CN(S(C)(=O)=O)CCN1c1ccc2c(cnn2-c2cc(O)c(F)c(F)c2)c1 ; CS(=O)(=O)C1CN(c2ccc3c(cnn3-c3cc(O)c(F)c(F)c3)c2)C1 ; O=S(=O)(C1CC1)N1CCN(c2cc3cnn(-c4cc(O)c(F)c(F)c4)c3cc2Cl)CC1] 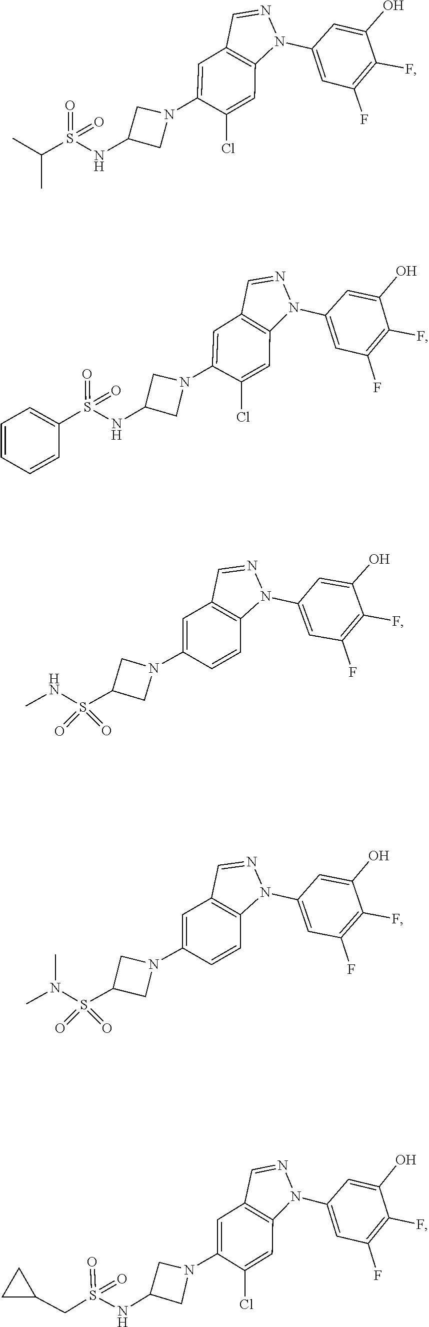 [SMILES: CC(C)S(=O)(=O)NC1CN(c2cc3cnn(-c4cc(O)c(F)c(F)c4)c3cc2Cl)C1 ; O=S(=O)(NC1CN(c2cc3cnn(-c4cc(O)c(F)c(F)c4)c3cc2Cl)C1)c1ccccc1 ; CNS(=O)(=O)C1CN(c2ccc3c(cnn3-c3cc(O)c(F)c(F)c3)c2)C1 ; CN(C)S(=O)(=O)C1CN(c2ccc3c(cnn3-c3cc(O)c(F)c(F)c3)c2)C1 ; O=S(=O)(CC1CC1)NC1CN(c2cc3cnn(-c4cc(O)c(F)c(F)c4)c3cc2Cl)C1] 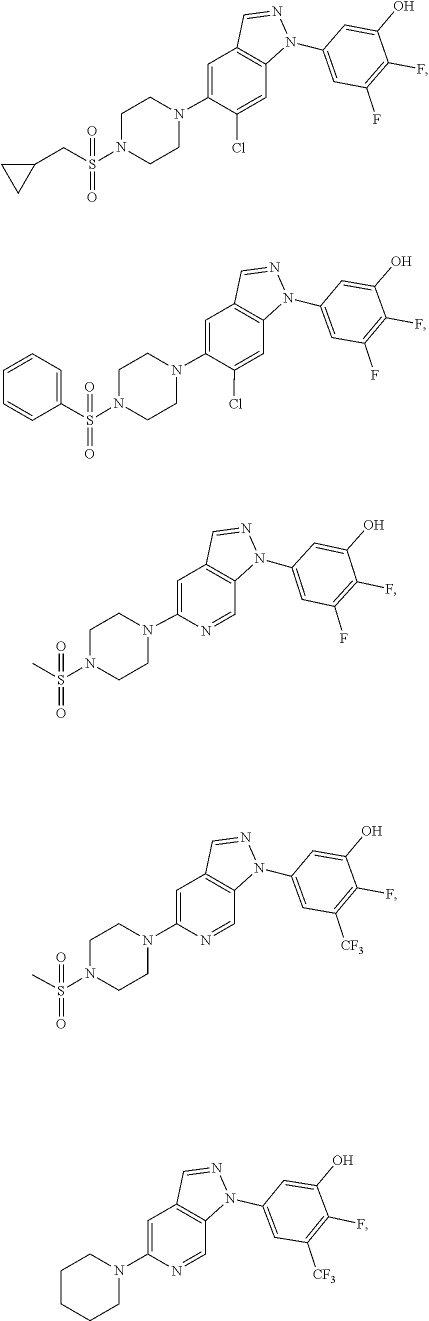 [SMILES: O=S(=O)(CC1CC1)N1CCN(c2cc3cnn(-c4cc(O)c(F)c(F)c4)c3cc2Cl)CC1 ; O=S(=O)(c1ccccc1)N1CCN(c2cc3cnn(-c4cc(O)c(F)c(F)c4)c3cc2Cl)CC1 ; CS(=O)(=O)N1CCN(c2cc3cnn(-c4cc(O)c(F)c(F)c4)c3cn2)CC1 ; CS(=O)(=O)N1CCN(c2cc3cnn(-c4cc(O)c(F)c(C(F)(F)F)c4)c3cn2)CC1 ; Oc1cc(-n2ncc3cc(N4CCCCC4)ncc32)cc(C(F)(F)F)c1F] 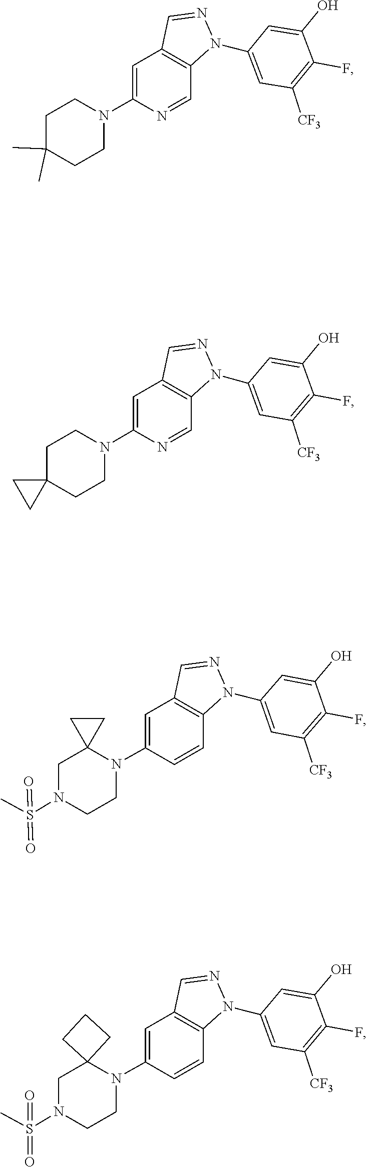 [SMILES: CC1(C)CCN(c2cc3cnn(-c4cc(O)c(F)c(C(F)(F)F)c4)c3cn2)CC1 ; Oc1cc(-n2ncc3cc(N4CCC5(CC4)CC5)ncc32)cc(C(F)(F)F)c1F ; CS(=O)(=O)N1CCN(c2ccc3c(cnn3-c3cc(O)c(F)c(C(F)(F)F)c3)c2)C2(CC2)C1 ; CS(=O)(=O)N1CCN(c2ccc3c(cnn3-c3cc(O)c(F)c(C(F)(F)F)c3)c2)C2(CCC2)C1] 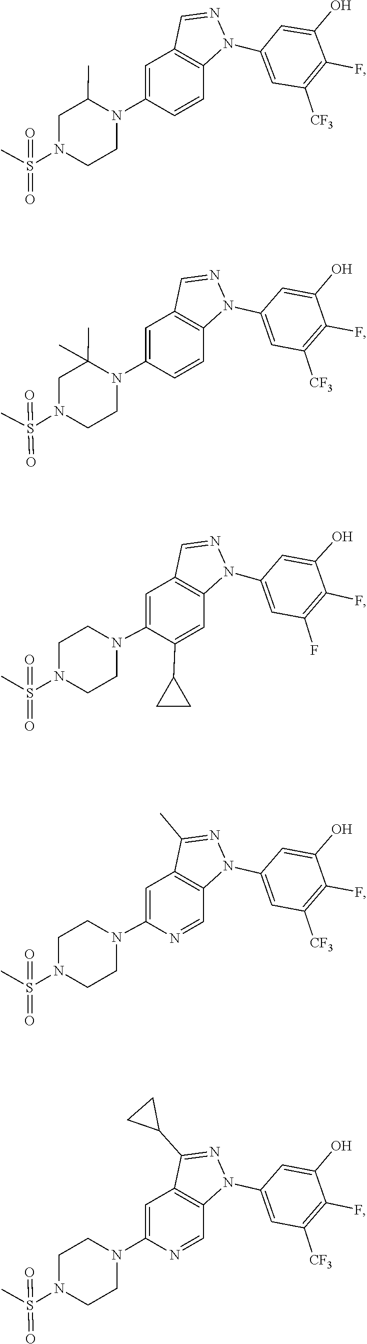 [SMILES: CC1CN(S(C)(=O)=O)CCN1c1ccc2c(cnn2-c2cc(O)c(F)c(C(F)(F)F)c2)c1 ; CC1(C)CN(S(C)(=O)=O)CCN1c1ccc2c(cnn2-c2cc(O)c(F)c(C(F)(F)F)c2)c1 ; CS(=O)(=O)N1CCN(c2cc3cnn(-c4cc(O)c(F)c(F)c4)c3cc2C2CC2)CC1 ; Cc1nn(-c2cc(O)c(F)c(C(F)(F)F)c2)c2cnc(N3CCN(S(C)(=O)=O)CC3)cc12 ; CS(=O)(=O)N1CCN(c2cc3c(C4CC4)nn(-c4cc(O)c(F)c(C(F)(F)F)c4)c3cn2)CC1] 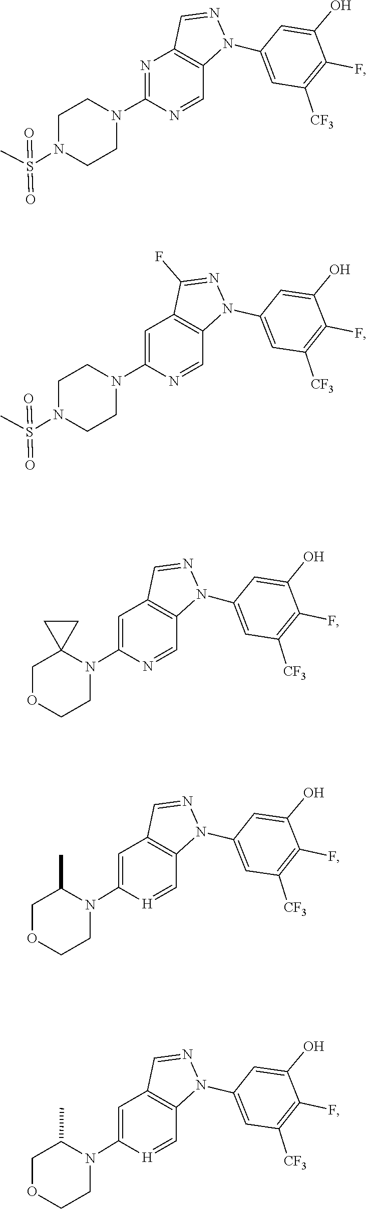 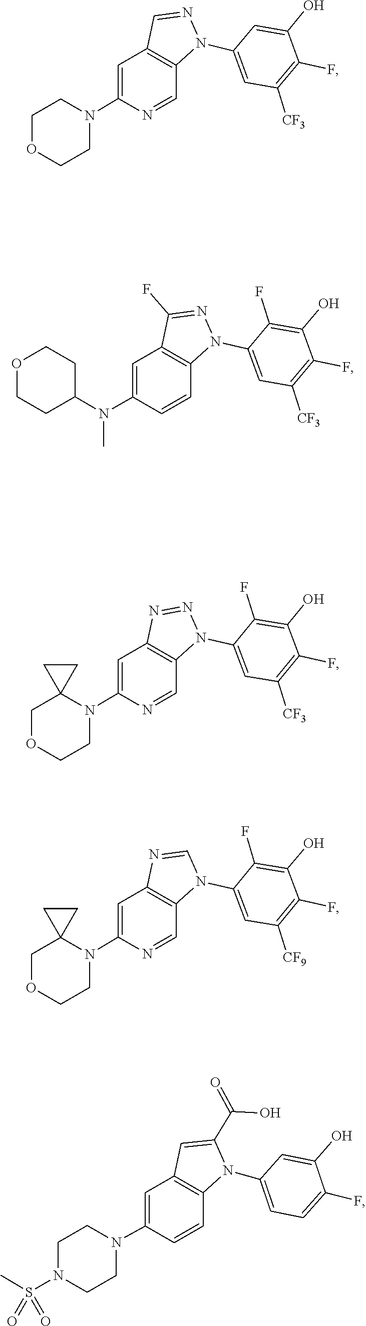 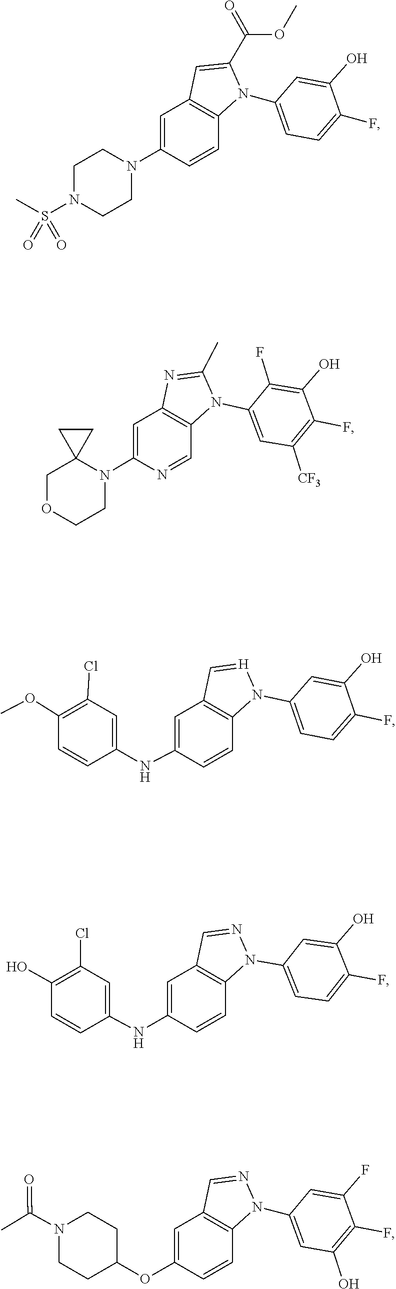 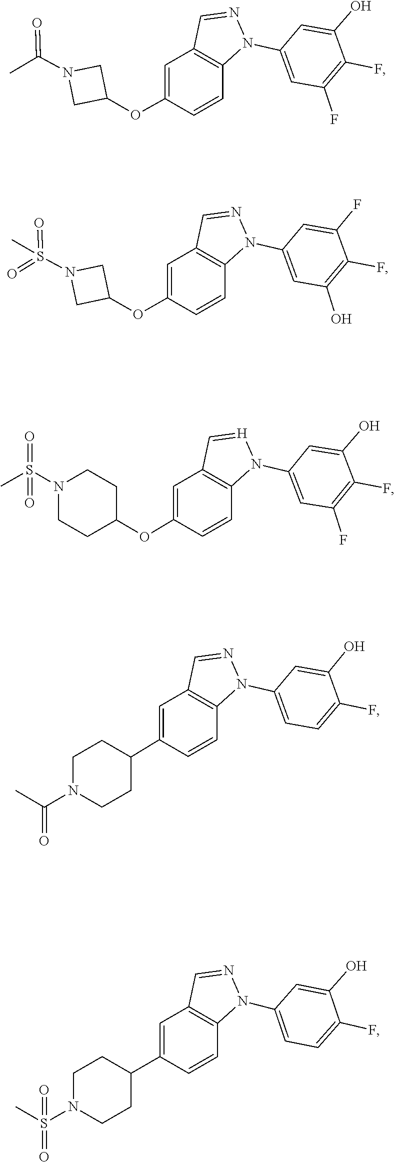 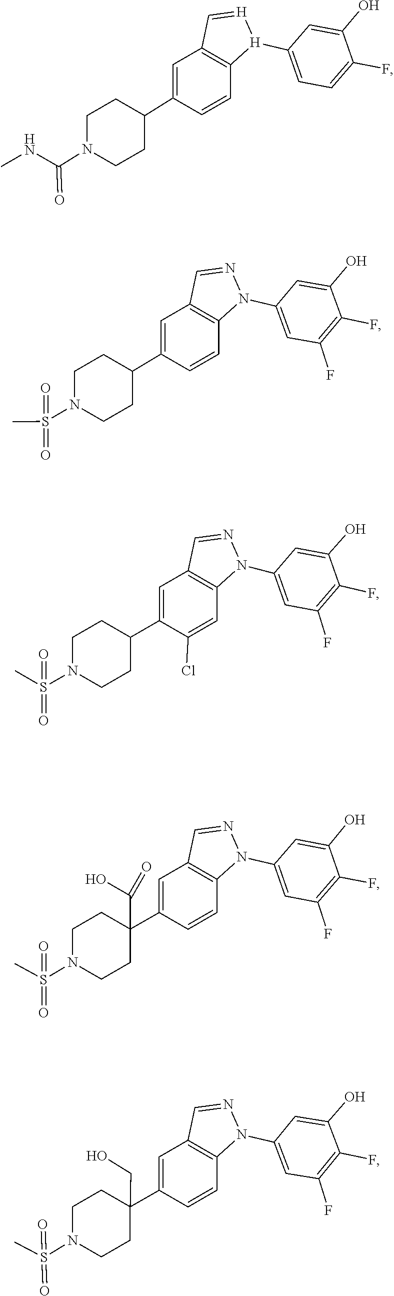 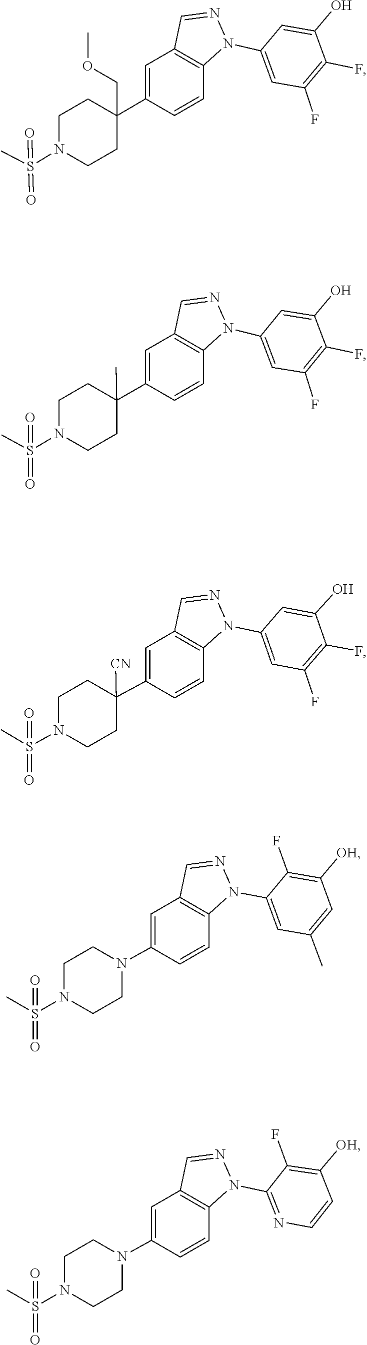 [SMILES: COCC1(c2ccc3c(cnn3-c3cc(O)c(F)c(F)c3)c2)CCN(S(C)(=O)=O)CC1 ; CC1(c2ccc3c(cnn3-c3cc(O)c(F)c(F)c3)c2)CCN(S(C)(=O)=O)CC1 ; CS(=O)(=O)N1CCC(C#N)(c2ccc3c(cnn3-c3cc(O)c(F)c(F)c3)c2)CC1 ; Cc1cc(O)c(F)c(-n2ncc3cc(N4CCN(S(C)(=O)=O)CC4)ccc32)c1 ; CS(=O)(=O)N1CCN(c2ccc3c(cnn3-c3nccc(O)c3F)c2)CC1] 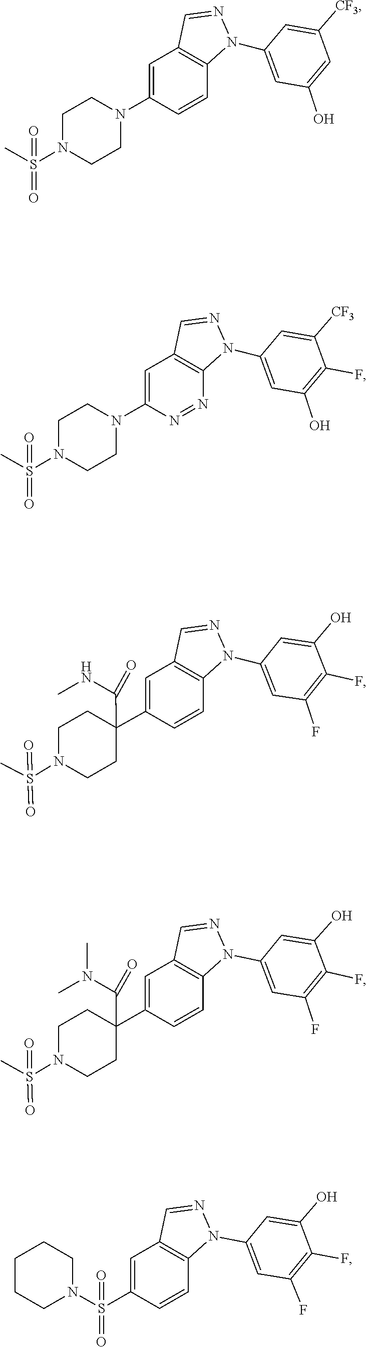 [SMILES: CS(=O)(=O)N1CCN(c2ccc3c(cnn3-c3cc(O)cc(C(F)(F)F)c3)c2)CC1 ; CS(=O)(=O)N1CCN(c2cc3cnn(-c4cc(O)c(F)c(C(F)(F)F)c4)c3nn2)CC1 ; CNC(=O)C1(c2ccc3c(cnn3-c3cc(O)c(F)c(F)c3)c2)CCN(S(C)(=O)=O)CC1 ; CN(C)C(=O)C1(c2ccc3c(cnn3-c3cc(O)c(F)c(F)c3)c2)CCN(S(C)(=O)=O)CC1 ; O=S(=O)(c1ccc2c(cnn2-c2cc(O)c(F)c(F)c2)c1)N1CCCCC1] 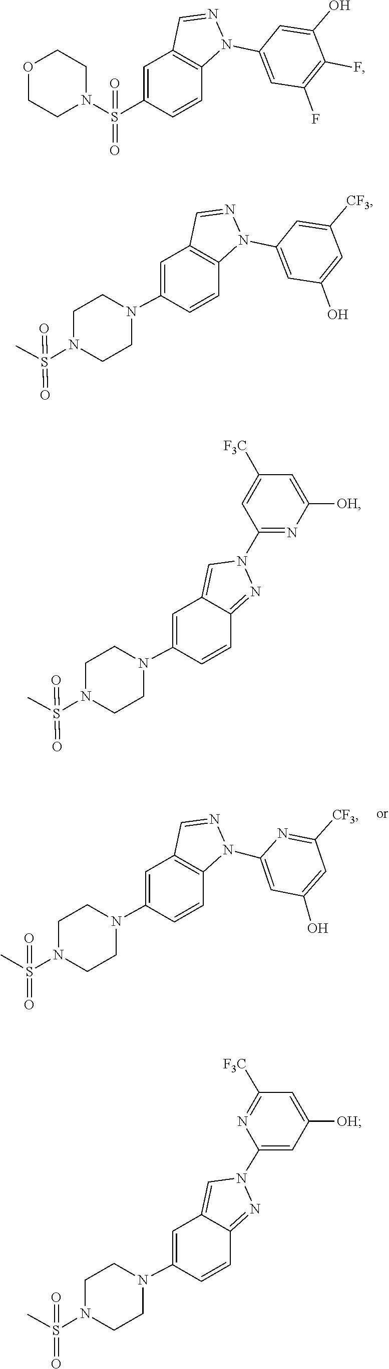 [SMILES: O=S(=O)(c1ccc2c(cnn2-c2cc(O)c(F)c(F)c2)c1)N1CCOCC1 ; CS(=O)(=O)N1CCN(c2ccc3c(cnn3-c3cc(O)cc(C(F)(F)F)c3)c2)CC1 ; CS(=O)(=O)N1CCN(c2ccc3nn(-c4cc(C(F)(F)F)cc(O)n4)cc3c2)CC1 ; CS(=O)(=O)N1CCN(c2ccc3c(cnn3-c3cc(O)cc(C(F)(F)F)n3)c2)CC1 ; "or" ; CS(=O)(=O)N1CCN(c2ccc3nn(-c4cc(O)cc(C(F)(F)F)n4)cc3c2)CC1]
or a pharmaceutically acceptable salt or solvate thereof.

38. A pharmaceutical composition comprising a compound of any one of claims 1-37, or a pharmaceutically acceptable salt or solvate thereof, and at least one pharmaceutically acceptable excipient.

39. The pharmaceutical composition of claim 38, wherein the pharmaceutical composition is formulated for administration to a mammal by intravenous administration, subcutaneous administration, oral administration, inhalation, nasal administration, dermal administration, or ophthalmic administration.

40. The pharmaceutical composition of claim 38, wherein the pharmaceutical composition is in the form of a tablet, a pill, a capsule, a liquid, a suspension, a gel, a dispersion, a solution, an emulsion, an ointment, or a lotion.

41. A method of treating or preventing a liver disease or condition in a mammal, comprising administering to the mammal a compound of any one of claims 1-37, or a pharmaceutically acceptable salt or solvate thereof.

42. The method of claim 41, wherein the liver disease or condition is an alcoholic liver disease or condition.

43. The method of claim 41, wherein the liver disease or condition is a nonalcoholic liver disease or condition.

44. The method of claim 41, wherein the liver disease or condition is liver inflammation, fatty liver (steatosis), liver fibrosis, hepatitis, cirrhosis, hepatocellular carcinoma, or combinations thereof.

45. The method of claim 41, wherein the liver disease or condition is primary biliary cirrhosis, primary sclerosing cholangitis, cholestasis, nonalcoholic steatohepatitis (NASH), nonalcoholic fatty liver disease (NAFLD), or combinations thereof.

46. A method of treating or preventing a disease or condition in a mammal that would benefit from treatment with an HSD17B13 inhibitor, comprising administering to the mammal a compound of any one of claims 1-37, or a pharmaceutically acceptable salt or solvate thereof.

47. The method of claim 46, wherein the disease or condition in the mammal that would benefit from treatment with an HSD17B13 inhibitor mammal is a liver disease or condition as described in claim 44 or claim 45.

48. A method of modulating hydroxysteroid 17β-dehydrogenase 13 (HSD17B13) activity in a mammal comprising administering to the mammal a compound of any one of claims 1-37, or a pharmaceutically acceptable salt or solvate thereof.

49. The method of claim 48, wherein modulating comprises inhibiting HSD17B13 activity.

50. The method of claim 48 or claim 49, wherein the mammal has a liver disease or condition as described in claim 44 or claim 45.
